SEQUENCE LISTING (167 sequences)

SEQ ID NO: 1 - protein, 313 aa, Homo sapiens
MLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

SEQ ID NO: 2 - protein, 313 aa, Mus musculus
MLLSKINSLAHLRARPCNDLHATKLAPGKEKEPLESQYQVGPLLGSGGFGSVYSGIRVADNLPVAIKHVEKDRISDWGELPNGTRVPMEVVLLKKVSSDFSGVIRLLDWFERPDSFVLILERPEPVQDLFDFITERGALQEDLARGFFWQVLEAVRHCHNCGVLHRDIKDENILIDLSRGEIKLIDFGSGALLKDTVYTDFDGTRVYSPPEWIRYHRYHGRSAAVWSLGILLYDMVCGDIPFEHDEEIIKGQVFFRQTVSSECQHLIKWCLSLRPSDRPSFEEIRNHPWMQGDLLPQAASEIHLHSLSPGSSK

SEQ ID NO: 3 - protein, 323 aa, Homo sapiens
MLTKPLQGPPAPPGTPTPPPGGKDREAFEAEYRLGPLLGKGGFGTVFAGHRLTDRLQVAIKVIPRNRVLGWSPLSDSVTCPLEVALLWKVGAGGGHPGVIRLLDWFETQEGFMLVLERPLPAQDLFDYITEKGPLGEGPSRCFFGQVVAAIQHCHSRGVVHRDIKDENILIDLRRGCAKLIDFGSGALLHDEPYTDFDGTRVYSPPEWISRHQYHALPATVWSLGILLYDMVCGDIPFERDQEILEAELHFPAHVSPDCCALIRRCLAPKPSSRPSLEEILLDPWMQTPAEDVTPQPLQRRPCPFGLVLATLSLAWPGLAPNG

SEQ ID NO: 4 - protein, 311 aa, Mus musculus
MLTKPLQGHPSPPVTPTQPPGGKDRAAFEAEYRLGPLLGKGGFGTVFAGHRVTDRRQVAIKVISRNRVLGWSTVSDSVTCPLEVALLWKVGEGNGHPGVIRLLDWFETPEGFMLVLERPMPAQDLFDYITEKGPLGESCSRSFFTQVVAAVQHCHARGVVHRDIKDENILIDLCRGSIKLIDFGSGALLHDEPYTDFDGTRVYSPPEWISRHQYHALPATVWSLGVLLYDMVCGDIPFERDQEILEAELHFPAHVSPDCCALIRRCLAPKPCSRPSLEEILLDPWMQSPAEEKPINSSKGSPTPLPWSLLP

SEQ ID NO: 5 - protein, 297 aa, Homo sapiens
MADKESFEKAYQVGAVLGSGGFGTVYAGSRIADGLPVAVKHVVKERVTEWGSLGGATVPLEVVLLRKVGAAGGARGVIRLLDWFERPDGFLLVLERPEPAQDLFDFITERGALDEPLARRFFAQVLAAVRHCHSCGVVHRDIKDENLLVDLRSGELKLIDFGSGALLKDTVYTDFDGTRVYSPPEWIRYHRYHGRSATVWSLGVLLYDMVCGDIPFEQDEEILRGRLLFRRRVSPECQQLIRWCLSLRPSERPSLDQIAAHPWMLGADGGAPESCDLRLCTLEPDDVASTTSSSESL

SEQ ID NO: 6 - protein, 326 aa, Mus musculus
MLLSKFGSLAHLCGPGGVDHLPVKILQPAKADKESFEKVYQVGAVLGSGGFGTVYAGSRIADGLPVAVKHVVKERVTEWGSLGGVAVPLEVVLLRKVGAAGGARGVIRLLDWFERPDGFLLVLERPEPAQDLFDFITERGALDEPLARRFFAQVLAAVRHCHNCGVVHRDIKDENLLVDLRSGELKLIDFGSGAVLKDTVYTDFDGTRVYSPPEWIRYHRYHGRSATVWSLGVLLYDMVCGDIPFEQDEEILRGRLFFRRRVSPECQQLIEWCLSLRPSERPSLDQIAAHPWMLGTEGSVPENCDLRLCALDTDDGASTTSSSESL

SEQ ID NO: 7 - protein, 323 aa, Coturnix coturnix
MLLSKFGSLAHICSPASMDHLPVKILPPVKVEKEPFDKVYQVGSVLGSGGFGTVYAGSRTADGLPVAVKHVVKERVTEWGTIGGVMVPLEIVLLKKVGSGFRGVIKLLDWYERPDGFLIVMERPELVKDLFDFITEKGALDEDTARGFFRQVLEAVRHCYGCGVVHRDIKDENLLVDLRTGELKLIDFGSGALLKDTVYTDFDGTRVYSPPEWIRYHRYHGRSATVWSLGVLLYDMVCGDIPFEQDEEILRGRLYFRRRISPECQQLIKWCLSLRPSDRPTLEQIFDHQWMHKSEVVKSEDCDIRLRTLDTDVSSTSSSNESL

SEQ ID NO: 8 - protein, 323 aa, Xenopus laevis
MLLSKFGSLAHICNPSNMEHLPVKILQPVKVDKEPFEKVYQVGSVVASGGFGTVYSDSRIADGQPVAVKHVAKERVTEWGTLNGVMVPLEIVLLKKVPTAFRGVINLLDWYERPDAFLIVMERPEPVKDLFDYITEKGPLDEDTARGFFRQVLEAVRHCYNCGVVHRDIKDENLLVDTRNGELKLIDFGSGALLKDTVYTDFDGTRVYSPPEWVRYHRYHGRSATVWSLGVLLYDMVYGDIPFEQDEEIVRVRLCFRRRISTECQQLIKWCLSLRPSDRPTLEQIFDHPWMCKCDLVKSEDCDLRLRTIDNDSSSTSSSNESL

SEQ ID NO: 9 - protein, 310 aa, Danio rerio
MLDKRIVDVRLDQLEILKAKNGKEHFEKQYTMGNLLGSGGFGSVYSGHRISDGQKVAIKQISRDRIQQWSKMPGEVNPVPNEIALLQSLGGGSGSVPGHRGIIRMLDWFEIPGQEYLIVFEKPQHCQDLFDFITERGRLDESLARRFLKQVIEAVQFCHSKGIVHRDIKDENILVDTRTGDIKVIDFGSGATLKDSMYTDFEGTRVYSPPEWILYHKYHALPLTVWSLGVLLYDMVCGDIPFEQDTDIVKAKPSFNKRISNDCRSLICSCLSYNPGDRPSLEQILQHPWMMESSVDNGDLQEESKIKPSL

SEQ ID NO: 10 - protein, 322 aa, Caenorhabditis elegans
MKKLASLQFFNLKLLLNGESSRGFSKFKKNYKLKAELGRGGFGVVYRAVRTCDNALVAVKFIERSNVKEWARINGEQVPMEICMLAKCSKVRGVIRLLDWYSIPEGFLIVMERPYPCIDMFDFIKGQGKISEDMARFLFRQIAVTVHECVQNRVLHRDLKDENIVIDLVTGSTKLIDFGAATVLRRSQYSDFQGTRLYCPPEWFLHSLYLGREAAVWSLGVLLYNSLNGRLPFRNEKDICTAHLLGPLPFFVPVSAEVKDLISKCLTFDPFQRCSLEAILNHPWVKQQTLSWDALTKNKVQKKTSESSDDHHSETLGDHSET

SEQ ID NO: 11 - protein, 328 aa, Caenorhabditis elegans
MLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFANSKGFLIVMERPANCMDLFDMVSVHGPLNEDMGKFIFKQVITTVFNMYSKHGLLHRDIKDENLIVNMNTGEVKLVDFGATAYAEKATKKEFQGTRSYCPPEWFRDQLYLPLEATSWSLGVLLFILLTGKLPFRNEIQICLGNVKFPPDLSKEVCQLVKSCLTTSTSARASLAQIAAHPWMETDKPFFGGDLTFEEALMEIKNPPVVIEDKEAEEEDN

SEQ ID NO: 12 - protein, 298 aa, Unknown Organism
  Description of Unknown Organism Mammalian protein sequence
TRDAALPGSHSTHGFYENYEPKEILGRGVSSVVRRCIHKPTCKEYAVKIIDVTGGGSFSAEEVQELREATLKEVDILRKVSGHPNIIQLKDTYETNTFFFLVFDLMKKGELFDYLTEKVTLSEKETRKIMRALLEVICALHKLNIVHRDLKPENILLDDDMNIKLTDFGFSCQLDPGEKLREVCGTPSYLAPEIIECSMNDNHPGYGKEVDMWSTGVIMYTLLAGSPPFWHRKQMLMLRMIMSGNYQFGSPEWDDYSDTVKDLVSRFLVVQPQKRYTAEEALAHPFFQQYVVEEVRHF

SEQ ID NO: 13 - protein, 332 aa, Unknown Organism
  Description of Unknown Organism Mammalian protein sequence
MPGAVEGPRWKQAEDIRDIYDFRDVLGTGAFSEVILAEDKRTQKLVAIKCIAKKALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGHQPGGTGTDS

SEQ ID NO: 14 - protein, 298 aa, Unknown Organism
  Description of Unknown Organism Mammalian protein sequence
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 15 - protein, 326 aa, Unknown Organism
  Description of Unknown Organism Mammalian protein sequence
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 16 - protein, 367 aa, Unknown Organism
  Description of Unknown Organism Mammalian protein sequence
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMXGXVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 17 - protein, 359 aa, Unknown Organism
  Description of Unknown Organism Mammalian protein sequence
SQERPTFYRQELNKTIWEVPERYQNLSPVGSGAYGSVCAAFDTKTGHRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLAQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 18 - protein, 411 aa, Unknown Organism
  Description of Unknown Organism Mammalian protein sequence
PTLDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGVVKGQPSPSAQVQQ

SEQ ID NO: 19 - protein, 355 aa, Unknown Organism
  Description of Unknown Organism Mammalian protein sequence
AAAAGPEMVRGQVFDVGPRYTNLSYIGEGAYGMVCSAYDNLNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS

SEQ ID NO: 20 - protein, 316 aa, Unknown Organism
  Description of Unknown Organism Mammalian protein sequence
SPRSTFSTLQELVDHYKKGNDGLCQKLSVPCMSSKPQKPWEKDAWEIPRESLKLEKKLGAGQFGEVWMATYNKHTKVAVKTMKPGSMSVEAFLAEANVMKTLQHDKLVKLHAVVTKEPIYIITEFMAKGSLLDFLKSDEGSKQPLPKLIDFSAQIAEGMAFIEQRNYIHRDLRAANILVSASLVCKIADFGLARVIEDNEYTAREGAKFPIKWTAPEAINFGSFTIKSDVWSFGILLMEIVTYGRIPYPGMSNPEVIRALERGYRMPRPENCPEELYNIMMRCWKNRPEERPTFEYIQSVLDDFYTATESQXEEIP

SEQ ID NO: 21 - protein, 279 aa, Unknown Organism
  Description of Unknown Organism Mammalian protein sequence
KPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEXTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 22 - protein, 319 aa, Unknown Organism
  Description of Unknown Organism Mammalian protein sequence
TSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQXQPGENL

SEQ ID NO: 23 - protein, 293 aa, Unknown Organism
  Description of Unknown Organism Mammalian protein sequence
GAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGT

SEQ ID NO: 24 - protein, 312 aa, Unknown Organism
  Description of Unknown Organism Mammalian protein sequence
GHMTPGMKIFIDPFTFEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSS

SEQ ID NO: 25 - protein, 278 aa, Unknown Organism
  Description of Unknown Organism Mammalian protein sequence
MGGSVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 26 - protein, 316 aa, Unknown Organism
  Description of Unknown Organism Mammalian protein sequence
MDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKVAPEDLYKDFLTLEHLICYSFQVAKGMEFLASRKCIHRDLAARNILLSEKNVVKICDFGLARDIYKDPDXVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQD

SEQ ID NO: 27 - protein, 310 aa, Unknown Organism
  Description of Unknown Organism Mammalian protein sequence
MVAGVSEYELPEDPRWELPRDRLVLGKPLGEGAFGQVVLAEAIGLDKDKPNRVTKVAVKMLKSDATEKDLSDLISEMEMMKMIGKHKNIINLLGACTQDGPLYVIVEYASKGNLREYLQARRPPGLEYSYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQE

SEQ ID NO: 28 - protein, 327 aa, Unknown Organism
  Description of Unknown Organism Mammalian protein sequence
MKKHHHHHHGKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 29 - protein, 299 aa, Unknown Organism
  Description of Unknown Organism Mammalian protein sequence
VPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIXETDXXRKGGKGLLPVRWMSPESLKDGVFTTYSDVWSFGVVLWEIATLAEQPYQGLSNEQVLRFVMEGGLLDKPDNCPDMLLELMRMCWQYNPKMRPSFLEIISSIKEEMEPGFREVSFYYSEENK

SEQ ID NO: 30 - protein, 306 aa, Unknown Organism
  Description of Unknown Organism Mammalian protein sequence
VFPSSVFVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIXETDXXRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 31 - protein, 352 aa, Unknown Organism
  Description of Unknown Organism Mammalian protein sequence
SKVTTVVATPGQGPDRPQEVSYTDTKVIGNGSFGVVYQAKLCDSGELVAIKKVLQGKAFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFAFPQIKAHPWTKVFRPRTPPEAIALCSRLLEYTPTARLTPLEACAHSFFDELRDPNVKLPNGRDTPALFNFTTQELSSNPPLATILIPPHARIQA

SEQ ID NO: 32 - protein, 321 aa, Unknown Organism
  Description of Unknown Organism Mammalian protein sequence
KELYEKYMIAEDLGRGEFGIVHRCVETSSKKTYMAKFVKVKGTDQVLVKKEISILNIARHRNILHLHESFESMEELVMIFEFISGLDIFERINTSAFELNEREIVSYVHQVCEALQFLHSHNIGHFDIRPENIIYQTRRSSTIKIIEFGQARQLKPGDNFRLLFTAPEYYAPEVHQHDVVSTATDMWSLGTLVYVLLSGINPFLAETNQQIIENIMNAEYTFDEEAFKEISIEAMDFVDRLLVKERKSRMTASEALQHPWLKQKIERVSTKVIRTLKHRRYYHTLIKKDLNMVVSAARISCGGAIRSQKGVSVAKVKVASI

SEQ ID NO: 33 - protein, 327 aa, Unknown Organism
  Description of Unknown Organism Mammalian protein sequence
SKARVYADVNVLRPKEYWDYEALTVQWGEQDDYEVVRKVGRGKYSEVFEGINVNNNEKCIIKILKPVKKKKIKREIKILQNLCGGPNIVKLLDIVRDQHSKTPSLIFEYVNNTDFKVLYPTLTDYDIRYYIYELLKALDYCHSQGIMHRDVKPHNVMIDHELRKLRLIDWGLAEFYHPGKEYNVRVASRYFKGPELLVDLQDYDYSLDMWSLGCMFAGMIFRKEPFFYGHDNHDQLVKIAKVLGTDGLNVYLNKYRIELDPQLEALVGRHSRKPWLKFMNADNQHLVSPEAIDFLDKLLRYDHQERLTALEAMTHPYFQQVRAAENS

SEQ ID NO: 34 - protein, 297 aa, Unknown Organism
  Description of Unknown Organism Mammalian protein sequence
SDEEILEKLRSIVSVGDPKKKYTRFEKIGQGASGTVYTAMDVATGQEVAIRQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLDMDVEKRGSAKELLQHQFLKIAKPLSSLTPLIAAAKEATKNNH

SEQ ID NO: 35 - protein, 373 aa, Unknown Organism
  Description of Unknown Organism Mammalian protein sequence
DYRPGGFHPAFKGEPYKDARYILVRKLGWGHFSTVWLAKDMVNNTHVAMKIVRGDKVYTEAAEDEIKLLQRVNDADNTKEDSMGANHILKLLDHFNHKGPNGVHVVMVFEVLGENLLALIKKYEHRGIPLIYVKQISKQLLLGLDYMHRRCGIIHTDIKPENVLMEIVDSPENLIQIKIADLGNACWYDEHYTNSIQTREYRSPEVLLGAPWGCGADIWSTACLIFELITGDFLFEPDEGHSYTKDDDHIAQIIELLGELPSYLLRNGKYTRTFFNSRGLLRNISKLKFWPLEDVLTEKYKFSKDEAKEISDFLSPMLQLDPRKRADAGGLVNHPWLKDTLGMEEIRVPDRELYGSGSDIPGWFEEVRDHKRH

SEQ ID NO: 36 - protein, 350 aa, Unknown Organism
  Description of Unknown Organism Mammalian protein sequence
GNAAAAKKGSEQESVKEFLAKAKEDFLKKWENPAQNTAHLDQFERIKTLGTGSFGRVMLVKHKETGNHFAMKILDKQKVVKLKQIEHTLNEKRILQAVNFPFLVKLEYSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKDGVNDIKNHKWFATTDWIAIYQRKVEAPFIPKFKGPGDTSNFDDYEEEEIRVSINEKCGKEFSEF

SEQ ID NO: 37 - protein, 342 aa, Unknown Organism
  Description of Unknown Organism Mammalian protein sequence
EDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 38 - protein, 317 aa, Unknown Organism
  Description of Unknown Organism Mammalian protein sequence
MELRVGNRYRLGRKIGSGSFGDIYLGTDIAAGEEVAIKLECVKTKHPQLHIESKIYKMMQGGVGIPTIRWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFATYLNFCRSLRFDDKPDYSYLRQLFRNLFHRQGFSYDYVFDWNMLKFGASRAADDAERERRDREERLRH

SEQ ID NO: 39 - protein, 303 aa, Unknown Organism
  Description of Unknown Organism Mammalian protein sequence
SKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 40 - protein, 300 aa, Unknown Organism
  Description of Unknown Organism Mammalian protein sequence
KKGNDGLCQKLSVPCMSSKPQKPWEKDAWEIPRESLKLEKKLGAGQFGEVWMATYNKHTKVAVKTMKPGSMSVEAFLAEANVMKTLQHDKLVKLHAVVTKEPIYIITEFMAKGSLLDFLKSDEGSKQPLPKLIDFSAQIAEGMAFIEQRNYIHRDLRAANILVSASLVCKIADFGLARVIEDNEYTAREGAKFPIKWTAPEAINFGSFTIKSDVWSFGILLMEIVTYGRIPYPGMSNPEVIRALERGYRMPRPENCPEELYNIMMRCWKNRPEERPTFEYIQSVLDDFYTATESQYQQQP

SEQ ID NO: 41 - protein, 312 aa, Unknown Organism
  Description of Unknown Organism Mammalian protein sequence
TKTSDGLCVKLGKPCLKIQVPAPFDLSYKTVDQWEIDRNSIQLLKRLGSGQFGEVWEGLWNNTTPVAVKTLKPGSMDPNDFLREAQIMKNLRHPKLIQLYAVCTLEDPIYIITELMRHGSLQEYLQNDTGSKIHLTQQVDMAAQVASGMAYLESRNYIHRDLAARNVLVGEHNIYKVADFGLARVFKVDNEDIYESRHEIKLPVKWTAPEAIRSNKFSIKSDVWSFGILLYEIITYGKMPYSGMTGAQVIQMLAQNYRLPQPSNCPQQFYNIMLECWNAEPKERPTFETLRWKLEDYFETDSSYSDANNFIR

SEQ ID NO: 42 - protein, 294 aa, Unknown Organism
  Description of Unknown Organism Mammalian protein sequence
TSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 43 - protein, 340 aa, Unknown Organism
  Description of Unknown Organism Mammalian protein sequence
ERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIP

SEQ ID NO: 44 - protein, 429 aa, Unknown Organism
  Description of Unknown Organism Mammalian protein sequence
IILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKF

SEQ ID NO: 45 - protein, 379 aa, Unknown Organism
  Description of Unknown Organism Mammalian protein sequence
TVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPC

SEQ ID NO: 46 - protein, 327 aa, Unknown Organism
  Description of Unknown Organism Mammalian protein sequence
MELRVGNRYRLGRKIGSGSFGDIYLGTDIAAGEEVAIKLECVKTKHPQLHIESKIYKMMQGGVGIPTIRWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFATYLNFCRSLRFDDKPDYSYLRQLFRNLFHRQGFSYDYVFDWNMLKFGASRAADDAERERRDREERLRHSRNPATRGLP

SEQ ID NO: 47 - protein, 396 aa, Unknown Organism
  Description of Unknown Organism Mammalian protein sequence
LDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGV

SEQ ID NO: 48 - protein, 326 aa, Unknown Organism
  Description of Unknown Organism Mammalian protein sequence
SPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLDMDVEKRGSAKELLQHQFLKIAKPLSSLTPLIAAAKEATKNNH

SEQ ID NO: 49 - protein, 349 aa, Unknown Organism
  Description of Unknown Organism Mammalian protein sequence
HHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 50 - protein, 370 aa, Unknown Organism
  Description of Unknown Organism Mammalian protein sequence
GSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGG

SEQ ID NO: 51 - protein, 298 aa, Unknown Organism; identical to SEQ ID NO: 14
  Description of Unknown Organism Mammalian protein sequence

SEQ ID NO: 52 - protein, 326 aa, Unknown Organism; identical to SEQ ID NO: 15
  Description of Unknown Organism Mammalian protein sequence

SEQ ID NO: 53 - protein, 385 aa, Unknown Organism
  Description of Unknown Organism Mammalian protein sequence
RTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKHPNGRDTPALFNFTTQELSSNPPLATILIPPHARIQAAAST

SEQ ID NO: 54 - protein, 298 aa, Unknown Organism
  Description of Unknown Organism Mammalian protein sequence
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWF

SEQ ID NO: 55 - protein, 326 aa, Unknown Organism
  Description of Unknown Organism Mammalian protein sequence
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQE

SEQ ID NO: 56 - protein, 360 aa, Unknown Organism [sequence omitted]

SEQ ID NO: 57 - protein, 343 aa, Unknown Organism
  Description of Unknown Organism Mammalian protein sequence
STVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTT

SEQ ID NO: 58 - protein, 351 aa, Unknown Organism
  Description of Unknown Organism Mammalian protein sequence
RANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 59 - protein, 349 aa, Unknown Organism
  Description of Unknown Organism Mammalian protein sequence
EPSEPTITKEDKTRAMNYDEEVDETREVSMTKASHSSTKELYEKYMIAEDLGRGEFGIVHRCVETSSKKTYMAKFVKVKGTDQVLVKKEISILNIARHRNILHLHESFESMEELVMIFEFISGLDIFERINTSAFELNEREIVSYVHQVCEALQFLHSHNIGHFDIRPENIIYQTRRSSTIKIIEFGQARQLKPGDNFRLLFTAPEYYAPEVHQHDVVSTATDMWSLGTLVYVLLSGINPFLAETNQQIIENIMNAEYTFDEEAFKEISIEAMDFVDRLLVKERKSRMTASEALQHPWLKQKIERVSTKVIRTLKHRRYYHTLIKKDLNMVVSAARISCGGAIRSQKGV

SEQ ID NO: 60 - protein, 358 aa, Unknown Organism
  Description of Unknown Organism Mammalian protein sequence
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEM

SEQ ID NO: 61 - protein, 338 aa, Unknown Organism
  Description of Unknown Organism Mammalian protein sequence
MTRDEALPDSHSAQDFYENYEPKEILGRGVSSVVRRCIHKPTSQEYAVKVIDVTGGGSFSPEEVRELREATLKEVDILRKVSGHPNIIQLKDTYETNTFFFLVFDLMKRGELFDYLTEKVTLSEKETRKIMRALLEVICTLHKLNIVHRDLKPENILLDDNMNIKLTDFGFSCQLEPGERLREVCGTPSYLAPEIIECSMNEDHPGYGKEVDMWSTGVIMYTLLAGSPPFWHRKQMLMLRMIMSGNYQFGSPEWDDYSDTVKDLVSRFLVVQPQNRYTAEEALAHPFFQQYLVEEVRHFSPRGKFKVIALTVLASVRIYYQYRRVKPVTREIVIRDPY

SEQ ID NO: 62 - protein, 374 aa, Unknown Organism
  Description of Unknown Organism Mammalian protein sequence
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIA

SEQ ID NO: 63 - protein, 303 aa, Unknown Organism
  Description of Unknown Organism Mammalian protein sequence
MEAPGLAQAAAAESDSRKVAEETPDGAPALCPSPEALSPEPPVYSLQDFDTLATVGTGTFGRVHLVKEKTAKHFFALKVMSIPDVIRLKQEQHVHNEKSVLKEVSHPFLIRLFWTWHDERFLYMLMEYVPGGELFSYLRNRGRFSSTTGLFYSAEIICAIEYLHSKEIVYRDLKPENILLDRDGHIKLTDFGFAKKLVDRTWTLCGTPEYLAPEVIQSKGHGRAVDWWALGILIFEMLSGFPPFFDDNPFGIYQKILAGKIDFPRHLDFHVKDLIKKLLVVDRTRRLGNMKNGANDVKHHRWF

SEQ ID NO: 64 - protein, 460 aa, Unknown Organism
  Description of Unknown Organism Mammalian protein sequence
IVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKD

SEQ ID NO: 65 - protein, 289 aa, Unknown Organism
  Description of Unknown Organism Mammalian protein sequence
VSNKAYEDAEAKAKYEAEAAFFANLKLSDFNIIDTLGVGGFGRVELVQLKSEESKTFAMKILKKRHIVDTRQQEHIRSEKQIMQGAHSDFIVRLYRTFKDSKYLYMLMEACLGGELWTILRDRGSFEDSTTRFYTACVVEAFAYLHSKGIIYRDLKPENLILDHRGYAKLVDFGFAKKIGFGKKTWTFCGTPEYVAPEIILNKGHDISADYWSLGILMYELLTGSPPFSGPDPMKTYNIILRGIDMIEFPKKIAKNAANLIKKLCRDNPSERLGNLKNGVKDIQKHKWF

SEQ ID NO: 66 - protein, 297 aa, Unknown Organism
  Description of Unknown Organism Mammalian protein sequence
QHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 67 - protein, 384 aa, Unknown Organism
  Description of Unknown Organism Mammalian protein sequence
MTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWI

SEQ ID NO: 68 - protein, 373 aa, Unknown Organism
  Description of Unknown Organism Mammalian protein sequence
GVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQNQLQLFRNFFLNSIYQCRDEANNSGVINESFEGEDGDVICLNSDDIMPVVLMETK

SEQ ID NO: 69 - protein, 292 aa, Unknown Organism
  Description of Unknown Organism Mammalian protein sequence
LSTQQPLTKKSGVVLHRAVPKDKWVLNHEDLVLGEQIGRGNFGEVFSGRLRADNTLVAVKSCRETLPPDLKAKFLQEARILKQYSHPNIVRLIGVCTQKQPIYIVMELVQGGDFLTFLRTEGARLRVKTLLQMVGDAAAGMEYLESKCCIHRDLAARNCLVTEKNVLKISDFGMSREEADGVYAASGGLRQVPVKWTAPEALNYGRYSSESDVWSFGILLWETFSLGASPYPNLSNQQTREFVEKGGRLPCPELCPDAVFRLMEQCWAYEPGQRPSFSTIYQELQSIRKRHR

SEQ ID NO: 70 - protein, 370 aa, Unknown Organism
  Description of Unknown Organism Mammalian protein sequence
LQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEE

SEQ ID NO: 71 - protein, 345 aa, Unknown Organism
  Description of Unknown Organism Mammalian protein sequence
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGARMYGVGVDMWAVGCILAELLLRVPFLPGDSDLDQLTRIFETLGTPTEEQWPDMCSLPDYVTFKSFPGIPLHHIFSAAGDDLLDLIQGLFLFNPCARITATQALKMKYFSNRPGPTPGCQLPRPNCPVETLKEQSNPALAIKRKRTEALEQGGLPKKLI

SEQ ID NO: 72 - protein, 360 aa, Unknown Organism
  Description of Unknown Organism Mammalian protein sequence
MAEPDLECEQIRLKCIRKEGFFTVPPEHRLGRCRSVKEFEKLNRIGEGTYGIVYRARDTQTDEIVALKKVRMDKEKDGIPISSLREITLLLRLRHPNIVELKEVVVGNHLESIFLVMGYCEQDLASLLENMPTPFSEAQVKCIVLQVLRGLQYLHRNFIIHRDLKVSNLLMTDKGCVKTADFGLARAYGVPVKPMTPKVVTLWYRAPELLLGTTTQTTSIDMWAVGCILAELLAHRPLLPGTSEIHQIDLIVQLLGTPSENIWPGFSKLPLVGQYSLRKQPYNNLKHKFPWLSEAGLRLLHFLFMYDPKKRATAGDCLESSYFKEKPLPCEPELMPTFPHHRNKRAAPATSEGQSKRCKP

SEQ ID NO: 73 - protein, 296 aa, Unknown Organism
  Description of Unknown Organism Mammalian protein sequence
RAQSLSHGLRLAAPCRKHEPEPLPHWDDWERPREEFTLCRKLGSGYFGEVFEGLWKDRVQVAIKVISRDNLLHQQMLQSEIQAMKKLRHKHILALYAVVSVGDPVYIITELMAKGSLLELLRDSDEKVLPVSELLDIAWQVAEGMCYLESQNYIHRDLAARNILVGENTLCKVGDFGLARLIKEDVYLSHDHNIPYKWTAPEALSRGHYSTKSDVWSFGILLHEMFSRGQVPYPGMSNHEAFLRVDAGYRMPCPLECPPSVHKLMLTCWCRDPEQRPCFKALRERLSSFTSYENPT

SEQ ID NO: 74 - protein, 321 aa, Unknown Organism
  Description of Unknown Organism Mammalian protein sequence
MATITCTRFTEEYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQETVDCLKKFNARRKLKGAILTTMLATRNFSGGKSGGN

SEQ ID NO: 75 - protein, 365 aa, Unknown Organism
  Description of Unknown Organism Mammalian protein sequence
MAKQYDSVECPFCDEVSKYEKLAKIGQGTFGEVFKARHRKTGQKVALKKVLMENEKEGFPITALREIKILQLLKHENVVNLIEICRTKASPYNRCKGSIYLVFDFCEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGERDYGPPIDLWGAGCIMAEMWTRSPIMQGNTEQHQLALISQLCGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQ

SEQ ID NO: 76 - protein, 324 aa, Unknown Organism
  Description of Unknown Organism Mammalian protein sequence
MAHSPVQSGLPGMQNLKADPEELFTKLEKIGKGSFGEVFKGIDNRTQKVVAIKIIDLEEAEDEIEDIQQEITVLSQCDSPYVTKYYGSYLKDTKLWIIMEYLGGGSALDLLEPGPLDETQIATILREILKGLDYLHSEKKIHRDIKAANVLLSEHGEVKLADFGVAGQLTDTQIKRNTFVGTPFWMAPEVIKQSAYDSKADIWSLGITAIELARGEPPHSELHPMKVLFLIPKNNPPTLEGNYSKPLKEFVEACLNKEPSFRPTAKELLKHKFILRNAKKTSYLTELIDRYKRWKAEQSHDDSSSEDSDAETDGQASGGSDSGD

SEQ ID NO: 77 - protein, 328 aa, Unknown Organism
  Description of Unknown Organism Mammalian protein sequence
MEQPPAPKSKLKKLSEDSLTKQPEEVFDVLEKLGEGSYGSVFKAIHKESGQVVAIKQVPVESDLQEIIKEISIMQQCDSPYVVKYYGSYFKNTDLWIVMEYCGAGSVSDIIRLRNKTLIEDEIATILKSTLKGLEYLHFMRKIHRDIKAGNILLNTEGHAKLADFGVAGQLTDTMAKRNTVIGTPFWMAPEVIQEIGYNCVADIWSLGITSIEMAEGKPPYADIHPMRAIFMIPTNPPPTFRKPELWSDDFTDFVKKCLVKNPEQRATATQLLQHPFIKNAKPVSILRDLITEAMEIKAKRHDEQQRELEEEEENSDEDELDSHTMVK

SEQ ID NO: 78 - protein, 322 aa, Unknown Organism
  Description of Unknown Organism Mammalian protein sequence
MELRDVSLQDPRDRFELLQRVGAGTYGDVYKARDTVTSELAAVKIVKLDPGDDISSLQQEITILRECRHPNVVAYIGSYLRNDRLWICMEFCGGGSLQEIYHATGPLEERQIAYVCRERLKGLHHLHSQGKIHRDIKGANLLLTLQGDVKLADFGVSGELTASVAKRRSFIGTPYWMAPEVAAVERKGGYNELCDVWALGITAIELGELQPPLFHLHPMRALMLMSKSSFQPPKLRDKTRWTQNFHHFLKLALTKNPKKRPTAEKLLQHPFTTQQLPRALLTQLLDKASDPHLGTPSPEDCELETYDMFPDTIHSRGQHGPA

SEQ ID NO: 79 - protein, 339 aa, Unknown Organism
  Description of Unknown Organism Mammalian protein sequence
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGE

SEQ ID NO: 80 - protein, 344 aa, Unknown Organism
  Description of Unknown Organism Mammalian protein sequence
MAFANFRRILRLSTFEKRKSREYEHVRRDLDPNEVWEIVGELGDGAFGKVYKAKNKETGALAAAKVIETKSEEELEDYIVEIEILATCDHPYIVKLLGAYYHDGKLWIMIEFCPGGAVDAIMLELDRGLTEPQIQVVCRQMLEALNFLHSKRIIHRDLKAGNVLMTLEGDIRLADFGVSAKNLKTLQKRDSFIGTPYWMAPEVVMCETMKDTPYDYKADIWSLGITLIEMAQIEPPHHELNPMRVLLKIAKSDPPTLLTPSKWSVEFRDFLKIALDKNPETRPSAAQLLEHPFVSSITSNKALRELVAEAKAEVMEEIEDGRDEGEEEDAVDAASTLENHTQNS

SEQ ID NO: 81 - protein, 337 aa, Unknown Organism
  Description of Unknown Organism Mammalian protein sequence
DGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 82 - protein, 345 aa, Unknown Organism
  Description of Unknown Organism Mammalian protein sequence
KNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 83 - protein, 376 aa, Unknown Organism
  Description of Unknown Organism Mammalian protein sequence
FFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWMEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITP

SEQ ID NO: 84 - protein, 300 aa, Unknown Organism
  Description of Unknown Organism Mammalian protein sequence
GLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFF

SEQ ID NO: 85 - protein, 308 aa, Unknown Organism
  Description of Unknown Organism Mammalian protein sequence
ACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFF

SEQ ID NO: 86 - protein, 310 aa, Unknown Organism
  Description of Unknown Organism Mammalian protein sequence
KSSRDPPSSPSSLSSPIQESTAPELPSETQETPGPALCSPLRKSPLTLEDFKFLAVLGRGHFGKVLLSEFRPSGELFAIKALKKGDIVARDEVESLMCEKRILAAVTSAGHPFLVNLFGCFQTPEHVCFVMEYSAGGDLMLHIHSDVFSEPRAIFYSACVVLGLQFLHEHKIVYRDLKLDNLLLDTEGYVKIADFGLCKEGMGYGDRTSTFCGTPEFLAPEVLTDTSYTRAVDWWGLGVLLYEMLVGESPFPGDDEEEVFDSIVNDEVRYPRFLSAEAIGIMRRLLRRNPERRLGSSERDAEDVKKQPFF

SEQ ID NO: 87 - protein, 309 aa, Unknown Organism
  Description of Unknown Organism Mammalian protein sequence
PIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFF

SEQ ID NO: 88 - protein, 305 aa, Unknown Organism
  Description of Unknown Organism Mammalian protein sequence
SRRSDSASSEPVGIYQGFEKKTGVAGEDMQDNSGTYGKIWEGSSKCNINNFIFHKVLGKGSFGKVLLGELKGRGEYSAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGMTGNIKIHPFF

SEQ ID NO: 89 - protein, 310 aa, Unknown Organism
  Description of Unknown Organism Mammalian protein sequence
LMELVPLDPENGQTSGEEAGLQPSKDEGVLKEISITHHVKAGSEKADPSHFELLKVLGQGSFGKVFLVRKVTRPDSGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHSHSADWWSYGVLMFEMLTGSLPFQGKDRKETMTLILKAKLGMPQFLSTEAQSLLRALFKRNPANRLGSGPDGAEEIKRHVFY

SEQ ID NO: 90 - protein, 317 aa, Unknown Organism
  Description of Unknown Organism Mammalian protein sequence
MDQYCILGRIGEGAHGIVFKAKHVETGEIIALKKVALRRLEDGFPNQALREIKALQEMEDNQYVVQLKAVFPHGGGFVLAFEFMLSDLAEVVRHAQRPLAQAQVKSYLQMLLKGVAFCHANNIVHRDLKPANLLISASGQLKIADFGLARVFSPDGSRLYTHQVATRSVGCIMGELLNGSPLFPGKNDIEQLCYVLRILGTPNPQVWPELTELPDYNKISFKEQVPMPLEEVLPDVSPQALDLLGQFLLYPPHQRIAASKALLHQYFFTAPLPAHPSELPIPQRLGGPAPKAHPGPPHIHDFHVDRPLEGVAVEPRA

SEQ ID NO: 91 - protein, 331 aa, Unknown Organism
  Description of Unknown Organism Mammalian protein sequence
MPAGGRAGSLKDPDVAELFFKDDPEKLFSDLREIGHGSFGAVYFARDVRNSEVVAIKKMSYSGKQSNEKWQDIIKEVRFLQKLRHPNTIQYRGCYLREHTAWLVMEYCLGSASDLLEVHKKPLQEVEIAAVTHGALQGLAYLHSHNMIHRDVKAGNILLSEPGLVKLGDFGSASIMAPANSFVGTPYWMAPEVILAMDEGQYDGKVDVWSLGITCIELAERKPPLFNMNAMSALYHIAQNESPVLQSGHWSEYFRNFVDSCLQKIPQDRPTSEVLLKHRFVLRERPPTVIMDLIQRTKDAVRELDNLQYRKMKKILFQEAPNGPGAEAPEE

SEQ ID NO: 92 - protein, 386 aa, Unknown Organism
  Description of Unknown Organism Mammalian protein sequence
SIPKLANSEKQGMRTHAVSVSETDDYAEIIDEEDTYTMPSTRDYEIQRERIELGRCIGEGQFGDVHQGIYMSPENPALAVAIKTCKNCTSDSVREKFLQEACHYTSLHWNWCRYISDPNVDACPDPRNAELTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESRRQATVSWDSGGSDEAPPKPSRPGYPSPRSSEG

SEQ ID NO: 93 - protein, 354 aa, Unknown Organism
  Description of Unknown Organism Mammalian protein sequence
PPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGSAHRRAGHRERGGVLPGPGPAVYLSRPPACPQGLYELMLRCWSRESEQRPPFSQLHRFLAEDALNTV

SEQ ID NO: 94 - protein, 325 aa, Unknown Organism [sequence omitted]

SEQ ID NO: 95 - protein, 324 aa, Unknown Organism
  Description of Unknown Organism Mammalian protein sequence
SQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFF

SEQ ID NO: 96 - protein, 374 aa, Unknown Organism
  Description of Unknown Organism Mammalian protein sequence
IVSQLGLTRDPLEEMAQLSSCDSPDTPETDDSIEGHGASLPSKKTPSEEDFETIKLISNGAYGAVFLVRHKSTRQRFAMKKINKQNLILRNQIQQAFVERDILTFAENPFVVSMFCSFDTKRHLCMVMEYVEGGDCATLLKNIGALPVDMVRLYFAETVLALEYLHNYGIVHRDLKPDNLLITSMGHIKLTDFGLSKMGLMSLTTNLYEGHIEKDAREFLDKQVCGTPEYIAPEVILRQGYGKPVDWWAMGIILYEFLVGCVPFFGDTPEELFGQVISDEIVWPEGDEALPPDAQDLTSKLLHQNPLERLGTGSAYEVKQHPFFTGLDWTGLLRQKAEFIPQLESEDDTSYFDTRSERYHHMDSEDEEEVSEDG

SEQ ID NO: 97 - protein, 338 aa, Unknown Organism
  Description of Unknown Organism Mammalian protein sequence
MMEKYEKIGKIGEGSYGVVFKCRNRDTGQIVAIKKFLESEDDPVIKKIALREIRMLKQLKHPNLVNLLEVFRRKRRLHLVFEYCDHTVLHELDRYQRGVPEHLVKSITWQTLQAVNFCHKHNCIHRDVKPENILITKHSVIKLCDFGFARLLTGPSDYYTDYVATRWYRSPELLVGDTQYGPPVDVWAIGCVFAELLSGVPLWPGKSDVDQLYLIRKTLGDLIPRHQQVFSTNQYFSGVKIPDPEDMEPLELKFPNISYPALGLLKGCLHMDPTERLTCEQLLHHPYFENIREIEDLAKEHDKPTRKTLRKSRKHHCFTETSKLQYLPQLTGSSILPA

SEQ ID NO: 98 - protein, 390 aa, Unknown Organism
  Description of Unknown Organism Mammalian protein sequence
GQQQPYFPSPAPGQAPGPAAAAPAQVQAAAAATVKAHHHQHSHHPQQQLDIEPDRPIGYGAFGVVWSVTDPRDGKRVALKKMPNVFQNLVSCKRVFRELKMLCFFKHDNVLSALDILQPPHIDYFEEIYVVTELMQSDLHKIIVSPQPLSSDHVKVFLYQILRGLKYLHSAGILHRDIKPGNLLVNSNCVLKICDFGLARVEELDESRHMTQEVVTQYYRAPEILMGSRHYSNAIDIWSVGCIFAELLGRRILFQAQSPIQQLDLITDLLGTPSLEAMRTACEGAKAHILRGPHKQPSLPVLYTLSSQATHEAVHLLCRMLVFDPSKRISAKDALAHPYLDEGRLRYHTCMCKCCFSTSTGRVYTSDFEPVTNPKFDDTFEKNLSSVRQV

SEQ ID NO: 99 - protein, 315 aa, Unknown Organism
  Description of Unknown Organism Mammalian protein sequence
MSKVFSGKRLEAEFPPHHSQSTFRKTSPAPGGPAGEGPLQSLTCLIGEKDLRLLEKLGDGSFGVVRRGEWDAPSGKTVSVAVKCLKPDVLSQPEAMDDFIREVNAMHSLDHRNLIRLYGVVLTPPMKMVTELAPLGSLLDRLRKHQGHFLLGTLSRYAVQVAEGMGYLESKRFIHRDLAARNLLLATRDLVKIGDFGLMRALPQNDDHYVMQEHRKVPFAWCAPESLKTRTFSHASDTWMFGVTLWEMFTYGQEPWIGLNGSQILHKIDKEGERLPRPEDCPQDIYNVMVQCWAHKPEDRPTFVALRDFLLEAQP

SEQ ID NO: 100 - protein, 359 aa, Unknown Organism
  Description of Unknown Organism Mammalian protein sequence
LGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQ

SEQ ID NO: 101 - protein, 370 aa, Unknown Organism
  Description of Unknown Organism Mammalian protein sequence
EPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGGQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCL

SEQ ID NO: 102 - protein, 362 aa, Unknown Organism
  Description of Unknown Organism Mammalian protein sequence
LTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKAMKYLASKKFVHRDLAARNCMLDEKFTVKVADFGLARDMYDKEYYSVHNKTGAKLPVKWMALESLQTQKFTTKSDVWSFGVVLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWET

SEQ ID NO: 103 - protein, 374 aa, Unknown Organism
  Description of Unknown Organism Mammalian protein sequence
QQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENAESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 104 - protein, 315 aa, Unknown Organism
  Description of Unknown Organism Mammalian protein sequence
NPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 105 - protein, 309 aa, Unknown Organism
  Description of Unknown Organism Mammalian protein sequence
NKLEEQNPDIVSRKKNQPTEVDPTHFEKRFLKRIRDLGEGHFGKVELCRYDPEDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 106 - protein, 328 aa, Unknown Organism
  Description of Unknown Organism Mammalian protein sequence
TTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 107 - protein, 361 aa, Unknown Organism
  Description of Unknown Organism Mammalian protein sequence
QTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSS

SEQ ID NO: 108 - protein, 280 aa, Unknown Organism
  Description of Unknown Organism Mammalian protein sequence
QWKWLEAQPMGEDWFLDFRVLGKGGFGEVSACQMKATGKLYACKKLNKKRLKKRKGYQGAMVEKKILMKVHSRFIVSLAYAFETKADLCLVMTIMNGGDIRYHIYNVNEENPGFPEPRALFYTAQIICGLEHLHQRRIVYRDLKPENVLLDNDGNVRISDLGLAVELLDGQSKTKGYAGTPGFMAPELLQGEEYDFSVDYFALGVTLYEMIAARGPFRARGEKVENKELKHRIISEPVKYPDKFSQASKDFCEALLEKDPEKRLGFRDETCDKLRAHPLF

SEQ ID NO: 109 - protein, 369 aa, Unknown Organism
  Description of Unknown Organism Mammalian protein sequence
GLEPLLDLLLGVHQELGASELAQDKYVADFLQWAEPIVVRLKEVRLQRDDFEILKVIGRGAFSEVAVVKMKQTGQVYAMKIMNKWDMLKRGEVSCFREERDVLVNGDRRWITQLHFAFQDENYLYLVMEYYVGGDLLTLLSKFGERIPAEMARFYLAEIVMAIDSVHRLGYVHRDIKPDNILLDRCGHIRLADFGSCLKLRADGTVRSLVAVGTPDYLSPEILQAVGGGPGTGSYGPECDWWALGVFAYEMFYGQTPFYADSTAETYGKIVHYKEHLSLPLVDEGVPEEARDFIQRLLCPPETRLGRGGAGDFRTHPFFFGLDWDGLRDSVPPFTPDFEGATDTCNFDLVEDGLTAMTLSDIREGAPLG

SEQ ID NO: 110 - protein, 395 aa, Unknown Organism
  Description of Unknown Organism Mammalian protein sequence
EEREMRQKKLEKVMEEEGLKDEEKRLRRSAHARKETEFLRLKRTRLGLEDFESLKVIGRGAFGEVRLVQKKDTGHVYAMKILRKADMLEKEQVGHIRAERDILVEADSLWVVKMFYSFQDKLNLYLIMEFLPGGDMMTLLMKKDTLTEEETQFYIAETVLAIDSIHQLGFIHRDIKPDNLLLDSKGHVKLSDFGLCTGLKKAHRTEFYRNLNHSLPSDFTFQNMNSKRKAETWKRNRRQLAFSTVGTPDYIAPEVFMQTGYNKLCDWWSLGVIMYEMLIGYPPFCSETPQETYKKVMNWKETLTFPPEVPISEKAKDLILRFCCEWEHRIGAPGVEEIKSNSFFEGVDWEHIRERPAAISIEIKSIDDTSNFDEFPESDILKPTVATSNHPETDY

SEQ ID NO: 111 - protein, 334 aa, Unknown Organism
  Description of Unknown Organism Mammalian protein sequence
MNRYTTMRQLGDGTYGSVLMGKSNESGELVAIKRMKRKFYSWDECMNLREVKSLKKLNHANVIKLKEVIRENDHLYFIFEYMKENLYQLMKDRNKLFPESVIRNIMYQILQGLAFIHKHGFFHRDMKPENLLCMGPELVKIADFGLARELRSQPPYTDYVSTRWYRAPEVLLRSSVYSSPIDVWAVGSIMAELYMLRPLFPGTSEVDEIFKICQVLGTPKKSDWPEGYQLASSMNFRFPQCVPINLKTLIPNASNEAIQLMTEMLNWDPKKRPTASQALKHPYFQVGQVLGPSSNHLESKQSLNKQLQPLESKPSLVEVEPKPLPDIIDQVVGQ

SEQ ID NO: 112 - protein, 335 aa, Unknown Organism
  Description of Unknown Organism Mammalian protein sequence
MKNYKAIGKIGEGTFSEVMKMQSLRDGNYYACKQMKQRFESIEQVNNLREIQALRRLNPHPNILMLHEVVFDRKSGSLALICELMDMNIYELIRGRRYPLSEKKIMHYMYQLCKSLDHIHRNGIFHRDVKPENILIKQDVLKLGDFGSCRSVYSKQPYTEYISTRWYRAPECLLTDGFYTYKMDLWSAGCVFYEIASLQPLFPGVNELDQISKIHDVIGTPAQKILTKFKQSRAMNFDFPFKKGSGIPLLTTNLSPQCLSLLHAMVAYDPDERIAAHQALQHPYFQEQRKTEKRALGSHRKAGFPEHPVAPEPLSNSCQISKEGRKQKQSLKQEE

SEQ ID NO: 113 - protein, 375 aa, Unknown Organism
  Description of Unknown Organism Mammalian protein sequence
QQAAPVTAAAAAAPAAATAAPAPAAPAAPAPAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDD

SEQ ID NO: 114 - protein, 339 aa, Unknown Organism
  Description of Unknown Organism Mammalian protein sequence
MEDDGKPRAPQAPLHSVVQQLHGKNLVFSDGYVVKETIGVGSYSECKRCVHKATNMEYAVKVIDKSKRDPSEEIEILLRYGQHPNIITLKDVYDDGKHVYLVTELMRGGELLDKILRQKFFSEREASFVLHTIGKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTPEEILTRIGSGKFTLSGGNWNTVSETAKDLVSKMLHVDPHQRLTAKQVLQHPWVTQKDKLPQSQLSHQDLQLVKGAMAATYSALNSSKPTPQLKPIESSILAQR

SEQ ID NO: 115 - protein, 357 aa, Unknown Organism
  Description of Unknown Organism Mammalian protein sequence
FPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKGCLHDKNSDQATWLTRL

SEQ ID NO: 116 - protein, 366 aa, Unknown Organism
  Description of Unknown Organism Mammalian protein sequence
MAEKFESLMNIHGFDLGSRYMDLKPLGCGGNGLVFSAVDNDCDKRVAIKKIVLTDPQSVKHALREIKIIRRLDHDNIVKVFEILGPSGSQLTDDVGSLTELNSVYIVQEYMETDLANVLEQGPLLEEHARLFMYQLLRGLKYIHSANVLHRDLKPANLFINTEDLVLKIGDFGLARIMDPHYSHKGHLSEGLVTKWYRSPRLLLSPNNYTKAIDMWAAGCIFAEMLTGKTLFAGAHELEQMQLILESIPVVHEEDRQELLSVIPVYIRNDMTEPHKPLTQLLPGISREALDFLEQILTFSPMDRLTAEEALSHPYMSIYSFPMDEPISSHPFHIEDEVDDILLMDETHSHIYNWERYHDCQFSEHD

SEQ ID NO: 117 - protein, 383 aa, Unknown Organism
  Description of Unknown Organism Mammalian protein sequence
MVSSQKLEKPIEMGSSEPLPIADGDRRRKKKRRGRATDSLPGKFEDMYKLTSELLGEGAYAKVQGAVSLQNGKEYAVKIIEKQAGHSRSRVFREVETLYQCQGNKNILELIEFFEDDTRFYLVFEKLQGGSILAHIQKQKHFNEREASRVVRDVAAALDFLHTKGIAHRDLKPENILCESPEKVSPVKICDFDLGSGMKLNNSCTPITTPELTTPCGSAEYMAPEVVEVFTDQATFYDKRCDLWSLGVVLYIMLSGYPPFVGHCGADCGWDRGEVCRVCQNKLFESIQEGKYEFPDKDWAHISSEAKDLISKLLVRDAKQRLSAAQVLQHPWVQGQAPEKGLPTPQVLQRNSSTMDLTLFAAEAIALNRQLSQHEENELAEEP

SEQ ID NO: 118 - protein, 325 aa, Unknown Organism
  Description of Unknown Organism Mammalian protein sequence
MTVFRQENVDDYYDTGEELGSGQFAVVKKCREKSTGLQYAAKFIKKRRTKSSRRGVSREDIEREVSILKEIQHPNVITLHEVYENKTDVILILELVAGGELFDFLAEKESLTEEEATEFLKQILNGVYYLHSLQIAHFDLKPENIMLLDRNVPKPRIKIIDFGLAHKIDFGNEFKNIFGTPEFVAPEIVNYEPLGLEADMWSIGVITYILLSGASPFLGDTKQETLANVSAVNYEFEDEYFSNTSALAKDFIRRLLVKDPKKRMTIQDSLQHPWIKPKDTQQALSRKASAVNMEKFKKFAARKKWKQSVRLISLCQRLSRSFLSR

SEQ ID NO: 119 - protein, 336 aa, Unknown Organism
  Description of Unknown Organism Mammalian protein sequence
VNDMGSASSSASLRVLGPGMDGIMVTWKDNFDSFYSEVAELGRGRFSVVKKCDQKGTKRAVATKFVNKKLMKRDQVTHELGILQSLQHPLLVGLLDTFETPTSYILVLEMADQGRLLDCVVRWGSLTEGKIRAHLGEVLEAVRYLHNCRIAHLDLKPENILVDESLAKPTIKLADFGDAVQLNTTYYIHQLLGNPEFAAPEIILGNPVSLTSDTWSVGVLTYVLLSGVSPFLDDSVEETCLNICRLDFSFPDDYFKGVSQKAKEFVCFLLQEDPAKRPSAALALQEQWLQAGNGRSTGVLDTSRLTSFIERRKHQNDVRPIRSIKNFLQSRLLPRV

SEQ ID NO: 120 - protein, 314 aa, Unknown Organism
  Description of Unknown Organism Mammalian protein sequence
HSLSTSSGSSIFTPEYDDSRIRRRGSDIDNPTLTVMDISPPSRSPRAPTNWRLGKLLGQGAFGRVYLCYDVDTGRELAVKQVQFDPDSPETSKEVNALECEIQLLKNFLHERIVQYYGCLRDPQEKTLSIFMEYMPGGSIKDQLKAYGALTENGTRKYTRQILEGVHYLHSNMILHRDIKGANILRDSTGNVKLGDFGASKRLQTICLSGTGMKSVTGTPYWMSPEVISGQGYGRKADIWSVACTVVEMLTEKPPWAEFEAMAAIFKIATQPTNPKLPPHVSDYTRDFLKRIFVEAKLRPSADELLRHMFVHYH

SEQ ID NO: 121 - protein, 321 aa, Unknown Organism
  Description of Unknown Organism Mammalian protein sequence
VNERDTVNHTTVDGYTEPHIQPTKSSSRQNIPRCRNSITSATDEQPHIGNYRLQKTIGKGNFAKVKLARHVLTGREVAVKIIDKTQLNPTSLQKLFREVRIMKILNHPNIVKLFEVIETEKTLYLVMEYASGGEVFDYLVAHGRMKEKEARAKFRQIVSAVQYCHQKYIVHRDLKAENLLLDGDMNIKIADFGFSNEFTVGNKLDTFCGSPPYAAPELFQGKKYDGPEVDVWSLGVILYTLVSGSLPFDGQNLKELRERVLRGKYRIPFYMSTDCENLLKKLLVLNPIKRGSLEQIMKDRWMNVGHEEEELKPYTEPDPDF

SEQ ID NO: 122 - protein, 320 aa, Unknown Organism
  Description of Unknown Organism Mammalian protein sequence
MATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRASIKDIREHEWFKQDLPKYLFPEDPSYSSTMIDDEALKEVCEKFECSEEEVLSCLYNRNHQD

SEQ ID NO: 123 - protein, 315 aa, Unknown Organism
  Description of Unknown Organism Mammalian protein sequence
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYS

SEQ ID NO: 124 - protein, 382 aa, Unknown Organism
  Description of Unknown Organism Mammalian protein sequence
LDMNGRCICPSLPYSPVSSPQSSPRLPRRPTVESHHVSITGMQDCVQLNQYTLKDEIGKGSYGVVKLAYNENDNTYYAMKVLSKKKLIRQAGFPRRPPPRGTRPAPGGCIQPRGPIEQVYQEIAILKKLDHPNVVKLVEVLDDPNEDHLYMVFELVNQGPVMEVPTLKPLSEDQARFYFQDLIKGIEYLHYQKIIHRDIKPSNLLVGEDGHIKIADFGVSNEFKGSDALLSNTVGTPAFMAPESLSETRKIFSGKALDVWAMGVTLYCFVFGQCPFMDERIMCLHSKIKSQALEFPDQPDIAEDLKDLITRMLDKNPESRIVVPEIKLHPWVTRHGAEPLPSEDENCTLVEVTEEEVENSVKHIPSLATVILVKTMIRKRSF

SEQ ID NO: 125 - protein, 359 aa, Unknown Organism
  Description of Unknown Organism Mammalian protein sequence
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDD

SEQ ID NO: 126 - protein, 353 aa, Unknown Organism
  Description of Unknown Organism Mammalian protein sequence
AAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLEPHKRRKALTEPEARYYLRQIVLGCQYLHRNRVIHRDLKLGNLFLNEDLEVKIGDFGLATKVEYDGERKKTLCGTPNYIAPEVLSKKEHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERP

SEQ ID NO: 127 - protein, 321 aa, Unknown Organism
  Description of Unknown Organism Mammalian protein sequence
TSVPHPVSRPLNNTQKSKQPLPSHLKIILRRNWHQNRKMKNQKEAVALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTANLYNRIANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 128 - protein, 380 aa, Unknown Organism
  Description of Unknown Organism Mammalian protein sequence
QSSAKRHLAEQFAVGEIITDMAKKEWKVGLPIGQGGFGCIYLADMNSSESVGSDAPCVVKVEPSDNGPLFTELKFYQRAAKPEQIQKWIRTRKLKYLGVPKYWGSGLHDKNGKSYRFMIMDRFGSDLQKIYEANAKRFSRKTVLQLSLRILDILEYIHEHEYVHGDIKASNLLLNYKNPDQVYLVDYGLAYRYCPEGVHKEYKEDPKRCHDGTIEFTSIDAHNGVAPSRRGDLEILGYCMIQWLTGHLPWEDNLKDPKYVRDSKIRYRENIASLMDKCFPEKNKPGEIAKYMETVKLLDYTEKPLYENLRDILLQGLKAIGSKDDGKLDLSVVENGGLKAKTITKKRKKEIEESKEPGVEDTEWSNTQTEEAIQTRSRTR

SEQ ID NO: 129 - protein, 328 aa, Unknown Organism
  Description of Unknown Organism Mammalian protein sequence
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTL

SEQ ID NO: 130 - protein, 376 aa, Unknown Organism
  Description of Unknown Organism Mammalian protein sequence
SAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELMFGCQVEGDAAETPPRPRTPGRPLSSYGMDSRPPMAIFELLDYIVNEPPPKLPSGVFSLEFQDFVNKCLIKNPAERADLKQLMVHAFIKRSDAEEVDFAGWLCSTIGLNQPSTPTHAAGV

SEQ ID NO: 131 - protein, 348 aa, Unknown Organism
  Description of Unknown Organism Mammalian protein sequence
WEAGHGGNVALPLKETSVENFLGATTPALAKLQVPREQLSEVLEQICSGSCGPIFRANMNTGDPSKPKSVILKALKEPAGLHEVQDFLGRIQFHQYLGKHKNLVQLEGCCTEKLPLYMVLEDVAQGDLLGFLWTCRRDVMTMDGLLYDLTEKQVYHIGKQVLLALEFLQEKHLFHGDVAARNILMQSDLTAKLCGLGLAYEVYTRGAISSTQTIPLKWLAPERLLLRPASIRADVWSFGILLYEMVTLGAPPYPEVPPTSILEHLQRRKIMKRPSSCTHTMYSIMKSCWRWREADRPSPRELRLRLEAAIKTADDEAVLQVPELVVPELYAAVAGIRVESLFYNYSML

SEQ ID NO: 132 - protein, 348 aa, Unknown Organism
  Description of Unknown Organism Mammalian protein sequence
PVIPKGSSVGTGTNLHRDISVSISVSNCQIQENVDISTVYQIFPDEVLGSGQFGIVYGGKHRKTGRDVAIKIIDKLRFPTKQESQLRNEVAILQNLHHPGVVNLECMFETPERVFVVMEKLHGDMLEMILSSEKGRLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWLQDYQTWLDLRELECKIGERYITHESDDLRWEKYAGEQRLQYPTHLINPSA

SEQ ID NO: 133 - protein, 374 aa, Unknown Organism
  Description of Unknown Organism Mammalian protein sequence
SSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRACKPLKEFMLSQDVEHERLFDLIQKMLEYDPAKRITLREALKHPFFDLLKKSI

SEQ ID NO: 134 - protein, 421 aa, Unknown Organism
  Description of Unknown Organism Mammalian protein sequence
EVYYAKKKRRHQQGRGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFENLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARS

SEQ ID NO: 135 - protein, 429 aa, Unknown Organism
  Description of Unknown Organism Mammalian protein sequence
EHPPMIQNNASGATVATATTSTATSKNSGSNSEGDYQLVQHEVLCSMTNTYEVLEFLGRGTFGQVVKCWKRGTNEIVAIKILKNRPSYARQGQIEVSILARLSTESADDYNFVRAYECFQHKNHTCLVFEMLEQNLYDFLKQNKFSPLPLKYIRPVLQQVATALMKLKSLGLIHADLKPENIMLVDPSRQPYRVKVIDFGSASHVSKAVCSTYLQSRYYRAPEIILGLPFCEAIDMWSLGCVIAELFLGWPLYPGASEYDQIRYISQTQGLPAEYLLSAGTKTTRFFNRDTDSPYPLWRLKTPDDHEAETGIKSKEARKYIFNCLDDMAQVNMTTDLEGSDMLVEKADRREFIDLLKKMLTIDADKRITPIETLNHPFVTMTHLLDFPHSTHVKSCFQNMEICKRRVNMYDTVNQSKTPFITHVAPSTS

SEQ ID NO: 136 - protein, 371 aa, Unknown Organism
  Description of Unknown Organism Mammalian protein sequence
DDMFAAYFDSARLRAAGIGKDFKENPNLRDNWTDAEGYYRVNIGEVLDKRYNVYGYTGQGVFSNVVRARDNARANQEVAVKIIRNNELMQKTGLKELEFLKKLNDADPDDKFHCLRLFRHFYHKQHLCLVFEPLSMNLREVLKKYGKDVGLHIKAVRSYSQQLFLALKLLKRCNILHADIKPDNILVNESKTILKLCDFGSASHVADNDITPYLVSRFYRAPEIIIGKSYDYGIDMWSVGCTLYELYTGKILFPGKTNNHMLKLAMDLKGKMPNKMIRKGVFKDQHFDQNLNFMYIEVDKVTEREKVTVMSTINPTKDLLADLIGCQRLPEDQRKKVHQLKDLLDQILMLDPAKRISINQALQHAFIQEKI

SEQ ID NO: 137 - protein, 336 aa, Unknown Organism
  Description of Unknown Organism Mammalian protein sequence
MSTASAASSSSSSSAGEMIEAPSQVLNFEEIDYKEIEVEEVVGRGAFGVVCKAKWRAKDVAIKQIESESERKAFIVELRQLSRVNHPNIVKLYGACLNPVCLVMEYAEGGSLYNVLHGAEPLPYYTAAHAMSWCLQCSQGVAYLHSMQPKALIHRDLKPPNLLLVAGGTVLKICDFGTACDIQTHMTNNKGSAAWMAPEVFEGSNYSEKCDVFSWGIILWEVITRRKPFDEIGGPAFRIMWAVHNGTRPPLIKNLPKPIESLMTRCWSKDPSQRPSMEEIVKIMTHLMRYFPGADEPLQYPCQYSDEGQSNSATSTGSFMDIASTNTSNKSDTNME

SEQ ID NO: 138 - protein, 330 aa, Unknown Organism
  Description of Unknown Organism Mammalian protein sequence
APAAPAGLQLPQEIPFHELQLEEIIGVGGFGKVYRALWRGEEVAVKAARLDPEKDPAVTAEQVCQEARLFGALQHPNIIALRGACLNPPHLCLVMEYARGGALSRVLAGRRVPPHVLVNWAVQVARGMNYLHNDAPVPIIHRDLKSINILILEAIENHNLADTVLKITDFGLAREWHKTTKMSAAGTYAWMAPEVIRLSLFSKSSDVWSFGVLLWELLTGEVPYREIDALAVAYGVAMNKLTLPIPSTCPEPFARLLEECWDPDPHGRPDFGSILKRLEVIEQSALFQMPLESFHSLQEDWKLEIQHMFDDLRTKEKELRSREEELLRAA

SEQ ID NO: 139 - protein, 312 aa, Unknown Organism
  Description of Unknown Organism Mammalian protein sequence
MSSLGASFVQIKFDDLQFFENCGGGSFGSVYRAKWISQDKEVAVKKLLKIEKEAEILSVLSHRNIIQFYGVILEPPNYGIVTEYASLGSLYDYINSNRSEEMDMDHIMTWATDVAKGMHYLHMEAPVKVIHRDLKSRNVVIAADGVLKICDFGASRFHNHTTHMSLVGTFPWMAPEVIQSLPVSETCDTYSYGVVLWEMLTREVPFKGLEGLQVAWLVVEKNERLTIPSSCPRSFAELLHQCWEADAKKRPSFKQIISILESMSNDTSLPDKCNSFLHNKAEWRCEIEATLERLKKLERDLSFKEQELKERE

SEQ ID NO: 140 - protein, 359 aa, Unknown Organism
  Description of Unknown Organism Mammalian protein sequence
CNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEY

SEQ ID NO: 141 - protein, 350 aa, Unknown Organism
  Description of Unknown Organism Mammalian protein sequence
NLSPTGWSQPKTPVPAQRERAPVSGTQEKNKIRPRGQRDSSYYWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF

SEQ ID NO: 142 - protein, 335 aa, Unknown Organism
  Description of Unknown Organism Mammalian protein sequence
MEGDGGTPWALALLRTFDAGEFTGWEKVGSGGFGQVYKVRHVHWKTWLAIKCSPSLHVDDRERMELLEEAKKMEMAKFRYILPVYGICREPVGLVMEYMETGSLEKLLASEPLPWDLRFRIIHETAVGMNFLHCMAPPLLHLDLKPANILLDAHYHVKISDFGLAKCNGLSHSHDLSMDGLFGTIAYLPPERIREKSRLFDTKHDVYSFAIVIWGVLTQKKPFADEKNILHIMVKVVKGHRPELPPVCRARPRACSHLIRLMQRCWQGDPRVRPTFQEITSETEDLCEKPDDEVKETAHDLDVKSPPEPRSEVVPARLKRASAPTFDNDYSLSEL

SEQ ID NO: 143 - protein, 359 aa, Unknown Organism
  Description of Unknown Organism Mammalian protein sequence
RQKPVLRSCSIDRSPGAGSLGSPASQRKDLGRSESLRVVCRPHRIFRPSDLIHGEVLGKGCFGQAIKVTHRETGEVMVMKELIRFDEETQRTFLKEVKVMRCLEHPNVLKFIGVLYKDKRLNFITEYIKGGTLRGIIKSMDSQYPWSQRVSFAKDIASGMAYLHSMNIIHRDLNSHNCLVRENKNVVVADFGLARLMVDEKTQPEGLRSLKKPDRKKRYTVVGNPYWMAPEMINGRSYDEKVDVFSFGIVLCEIIGRVNADPDYLPRTMDFGLNVRGFLDRYCPPNCPPSFFPITVRCCDLDPEKRPSFVKLEHWLETLRMHLAGHLPLGPQLEQLDRGFWETYRRGESGLPAHPEVPD

SEQ ID NO: 144 - protein, 358 aa, Unknown Organism
  Description of Unknown Organism Mammalian protein sequence
PLRGPGPGPGEVPGEGPPGPGGTGGGPGRGRPSSYRVLRSAVSSLARVDDFHCAEKIGAGFFSEVYKVRHRQSGQVMVLKMNKLPSNRGNTLREVQLMNRLRHPNILRFMGVCVHQGQLHALTEYMNGGTLEQLLSSPEPLSWPVRLHLALDIARGLRYLHSKGVFHRDLTSKNCLVRREDRGFTAVVGDFGLAEKIPVYREGARKEPLAVVGSPYWMAPEVLRGELYDEKADVFAFGIVLCELIARVPADPDYLPRTEDFGLDVPAFRTLVGDDCPLPFLLLAIHCCNLEPSTRAPFTEITQHLEWILEQLPEPAPLTRTALTHNQGSVARGGPSATLPRPDPRLSRSRSDLFLPPS

SEQ ID NO: 145 - protein, 325 aa, Unknown Organism
  Description of Unknown Organism Mammalian protein sequence
TPVQNLEQSYMPPDSSSPENKSLEVSDTRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSGASQCRHEKKNKSPDIKKVHQLLQEMTAS

SEQ ID NO: 146 - protein, 402 aa, Unknown Organism
  Description of Unknown Organism Mammalian protein sequence
PSPASLWPPPPSPAPSSTKPGPESSVSLLQGARPSPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQVYERLEKLQAVVAGVPGHLEAASCIPPSPQENSYVSSTGRAHSGAAPWQP

SEQ ID NO: 147 - protein, 329 aa, Unknown Organism
  Description of Unknown Organism Mammalian protein sequence
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLIVEPMENWLQLMLNWDPQQRGGPVDLTLKQPRCFVLMDHILNLKIVHILNMTSAKIISFLLP

SEQ ID NO: 148 - protein, 347 aa, Unknown Organism
  Description of Unknown Organism Mammalian protein sequence
MQSTANYLWHTDDLLGQGATASVYKARNKKSGELVAVKVFNTTSYLRPREVQVREFEVLRKLNHQNIVKLFAVEETGGSRQKVLVMEYCSSGSLLSVLESPENAFGLPEDEFLVVLRCVVAGMNHLRENGIVHRDIKPGNIMRLVGEEGQSIYKLTDFGAARELDDDEKFVSVYGTEEYLHPDMYERAVLRKPQQKAFGVTVDLWSIGVTLYHAATGSLPFIPFGGPRRNKEIMYRITTEKPAGAIAGAQRRENGPLEWSYTLPITCQLSLGLQSQLVPILANILEVEQAKCWGFDQFFAETSDILQRVVVHVFSLSQAVLHHIYIHAHNTIAIFQEAVHKQTSVAP

SEQ ID NO: 149 - protein, 373 aa, Unknown Organism
  Description of Unknown Organism Mammalian protein sequence
IDFTPPAEDTPSVQSPAEVFTLSVPNISLPAPSQFQPSVEGLKSQVARHSLNYIQEIGNGWFGKVLLGEIYTGTSVARVIVKELKASANPKEQDTFLKNGEPYYILQHPNILQCVGQCVEAIPYLLVFEFCDLGDLKAYLRSEQEHMRGDSQTMLLQRMACEVAAGLAAMHKLHFLHSDLALRNCFLTSDLNVKVGDYGIGFSRYKEDYIETDDKKVFPLRWTAPELVTSFQDRLLTADQTKYSNIWSLGVTLWELFDNAAQPYSNLSNLDVLNQVIRERDTKLPKPQLEQPYSDRWYEVLQFCWLSPEKRPAAEDVHRLLTYLRLQSQRDSEVDFEQQWNALKPNTNSRDSSNNAAFPILDHFARDRLGREM

SEQ ID NO: 150 - protein, 321 aa, Unknown Organism
  Description of Unknown Organism Mammalian protein sequence
MPSRAEDYEVLYTIGTGSYGRCQKIRRKSDGKILVWKELDYGSMTEAEKQMLVSEVNLLRELKHPNIVRYYDRIIDRTNTTLYIVMEYCEGGDLASVITKGTKERQYLDEEFVLRVMTQLTLALKECHRRSDGGHTVLHRDLKPANVFLDGKQNVKLGDFGLARILNHDTSFAKTFVGTPYYMSPEQMNRMSYNEKSDIWSLGCLLYELCALMPPFTAFSQKELAGKIREGKFRRIPYRYSDELNEIITRMLNLKDYHRPSVEEILENPLIADLVADEQRRNLERRGRQLGEPEKSQDSSPVLSELKLKEIQLQERERALK

SEQ ID NO: 151 - protein, 335 aa, Unknown Organism
  Description of Unknown Organism Mammalian protein sequence
SNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGFDYDPETSDDSLESSDYDPENSKNSSRSKTKCLFIQMEFCDKGTLEQWIEKRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

SEQ ID NO: 152 - protein, 313 aa, Unknown Organism; identical to SEQ ID NO: 1
  Description of Unknown Organism Mammalian protein sequence

SEQ ID NO: 153 - protein, 361 aa, Unknown Organism
  Description of Unknown Organism Mammalian protein sequence
RLGHLKKEEAGIQAELERLERVRNLHIRELKRIHNEDNSQFKDHPTLNERYLLLHLLGRGGFSEVDKAFDLSEQRYAAVKIHQLNKSWRDEKKENYHKHACREYRIHKELDHPRIVKLYDYFSLDTDTFCTVLEYCEGNDLDFYLKQHKLMSEKEARSIVMQIVNALRYLNEIKPPIIHYDLKPGNILLVDGTACGEIKITDFGLSKIMDDDSYGVDGMVLTSQGAGTYWYLPPECFVVGKEPPKISNKVDVWSVGVIFYQCLYGRKPFGHNQSQQDILQENTILKATEVQFPVKPVVSSEAKAFIRRCLAYRKEDRFDVHQLACDPYLLPHMRRSNSSGNLHMAGLTASPTPPSSSIITY

SEQ ID NO: 154 - protein, 359 aa, Unknown Organism
  Description of Unknown Organism Mammalian protein sequence
LVGSKEEPPPARSGSGGGSAKEPQEERSQQQDDIEELETKAVGMSNDGRFLKFDIEIGRGSFKTVYKGLDTETTVEVAWCELQDRKLTKSERQRFKEEAEMLKGLQHPNIVRFYDSWESTVKGKKCIVLVTELMTSGTLKTYLKRFKVMKIKVLRSWCRQILKGLQFLHTRTPPIIHRDLKCDNIFITGPTGSVKIGDLGLATLKRASFAKSVIGTPEFMAPEMYEEKYDESVDVYAFGMCMLEMATSEYPYSECQNAAQIYRRVTSGVKPASFDKVAIPEVKEIIEGCIRQNKDERYSIKDLLNHAFFQEETGVRVELAEEDDGEKIAIKLWLRIEDIKKLKGKYKDNEAIEFSFDLE

SEQ ID NO: 155 - protein, 350 aa, Unknown Organism
  Description of Unknown Organism Mammalian protein sequence
SIPISRLFPPRTPGWHQLQPRRVSFRGEASETLQSPGYDPSRPESFFQQSFQRLSRLGHGSYGEVFKVRSKEDGRLYAVKRSMSPFRGPKDRARKLAEVGSHEKVGQHPCCVRLEQAWEEGGILYLQTELCGPSLQQHCEAWGASLPEAQVWGYLRDTLLALAHLHSQGLVHLDVKPANIFLGPRGRCKLGDFGLLVELGTAGAGEVQEGDPRYMAPELLQGSYGTAADVFSLGLTILEVACNMELPHGGEGWQQLRQGYLPPEFTAGLSSELRSVLVMMLEPDPKLRATAEALLALPVLRQPRAWGVLWCMAAEALSRGWALWQALLALLCWLWHGLAHPASWLQPLGP

SEQ ID NO: 156 - protein, 371 aa, Unknown Organism
  Description of Unknown Organism Mammalian protein sequence
GQCRRRKRTYWNDSCGEDMEASDYELEDETRPAKRITITESNMKSRYTTEFHELEKIGSGEFGSVFKCVKRLDGCIYAIKRSKKPLAGSVDEQNALREVYAHAVLGQHSHVVRYFSAWAEDDHMLIQNEYCNGGSLADAISENYRIMSYFKEAELKDLLLQVGRGLRYIHSMSLVHMDIKPSNIFISRTSIPNAASEEGDEDDWASNKVMFKIGDLGHVTRISSPQVEEGDSRFLANEVLQENYTHLPKADIFALALTVVCAAGAEPLPRNGDQWHEIRQGRLPRIPQVLSQEFTELLKVMIHPDPERRPSAMALVKHSVLLSASRKSAEQLRIELNAEKFKNSLLQKELKKAQMAKAAAEERALFTDRMA

SEQ ID NO: 157 - protein, 351 aa, Unknown Organism
  Description of Unknown Organism Mammalian protein sequence
ANHISNTAKHFYGQRPQESGILLNMVITPQNGRYQIDSDVLLIPWKLTYRNIGSDFIPRGAFGKVYLAQDIKTKKRMACKLIPVDQFKPSDVEIQACFRHENIAELYGAVLWGETVHLFMEAGEGGSVLEKLESCGPMREFEIIWVTKHVLKGLDFLHSKKVIHHDIKPSNIVFMSTKAVLVDFGLSVQMTEDVYFPKDLRGTEIYMSPEVILCRGHSTKADIYSLGATLIHMQTGTPPWVKRYPRSAYPSYLYIIHKQAPPLEDIADDCSPGMRELIEASLERNPNHRPRAADLLKHEALNPPREDQPRCTSLDSALLERKRLLSRKELELPENIADSSCTGSTEESEML

SEQ ID NO: 158 - protein, 343 aa, Unknown Organism
  Description of Unknown Organism Mammalian protein sequence
TDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSRKKNQPTEVDPTHFEKRF

SEQ ID NO: 159 - protein, 367 aa, Unknown Organism
  Description of Unknown Organism Mammalian protein sequence
FPPACQLSTPYGQPACFQQQQHQILATPLQNLQVLASSSANECISVKGRIYSILKQIGSGGSSKVFQVLNEKKQIYAIKYVNLEEADNQTLDSYRNEIAYLNKLQQHSDKIIRLYDYEITDQYIYMVMECGNIDLNSWLKKKKSIDPWERKSYWKNMLEAVHTIHQHGIVHSDLKPANFLIVDGMLKLIDFGIANQMQPDTTSVVKDSQVGTVNYMPPEAIKDMSSSRENGKSKSKISPKSDVWSLGCILYYMTYGKTPFQQIINQISKLHAIIDPNHEIEFPDIPEKDLQDVLKCCLKRDPKQRISIPELLAHPYVQIQTHPVNQMAKGTTEEMKYVLGQLVGLNSPNSILKAAKTLYEHYSGGES

SEQ ID NO: 160 - protein, 337 aa, Unknown Organism
  Description of Unknown Organism Mammalian protein sequence
YLRSEFSPSVDARPCSSPSELPAKLLLGATLPRAPRLPRRLAWCSIDWEQVCLLQRLGAGGFGSVYKATYRGVPVAIKQVNKCTKNRLASRRSFWAELNVARLRHDNIVRVVAASTRTPAGSNSLGTIIMEFGGNVTLHQVIYGAAGHPEGDAGEPHCRTGGQLSLGKCLKYSLDVVNGLLFLHSQSIVHLDLKPANILISEQDVCKISDFGCSEKLEDLLCFQTPSYPLGGTYTHRAPELLKGEGVTPKADIYSFAITLWQMTTKQAPYSGERQHILYAVVAYDLRPSLSAAVFEDSLPGQRLGDVIQRCWRPSAAQRPSARLLLVDLTSLKAELG

SEQ ID NO: 161 - protein, 362 aa, Unknown Organism
  Description of Unknown Organism Mammalian protein sequence
SGTSSPSTSPRASNHSLCSGSSASKAGSSPSLEQDDGDEETSVVIVGKISFCPKDVLGHGAEGTIVYRGMFDNRDVAVKRILPECFSFADREVQLLRESDEHPNVIRYFCTEKDRQFQYIAIELCAATLQEYVEQKDFAHLGLEPITLLQQTTSGLAHLHSLNIVHRDLKPHNILISMPNAHGKIKAMISDFGLCKKLAVGRHSFSRRSGVPGTEGWIAPEMLSEDCKENPTYTVDIFSAGCVFYYVVSEGSHPFGKSLQRQANILLGACSLDCLHPEKHEDVIARELIEKMIAMDPQKRPSANDVLKHPFFWSLEKQLQFFQDVSDRIEKESLDGPIVKQLERGGRAVVKMDWRENITDPL

SEQ ID NO: 162 - protein, 310 aa, Unknown Organism
  Description of Unknown Organism Mammalian protein sequence
MGHALCVCSRGTVIIDNKRYLFIQKLGEGGFSYVDLVEGLHDGHFYALKRILCHEQQDREEAQREADMHRLFNHPNILRLVAYCLRERGAKHEAWLLLPFFKRGTLWNEIERLKDKGNFLTEDQILWLLLGICRGLEAIHAKGYAHRDLKPTNILLGDEGQPVLMDLGSMNQACIHVEGSRQALTLQDWAAQRCTISYRAPELFSVQSHCVIGERTDVWSLGCVLYAMMFGEGPYDMVFQKGDSVALAVQNQLSIPQSPRHSSALRQLLNSMMTVDPHQRPHIPLLLSQLEALQPPAPGQHTTQIEKAAC

SEQ ID NO: 163 - protein, 365 aa, Unknown Organism
  Description of Unknown Organism Mammalian protein sequence
MSLLQSALDFLAGPGSLGGASGRDQSDFVGQTVELGELRLRVRRVLAEGGFAFVYEAQDVGSGREYALKRLLSNEEEKNRAIIQEVCFMKKLSGHPNIVQFCSAASIGKEESDTGQAEFLLLTELCKGQLVEFLKKMESRGPLSCDTVLKIFYQTCRAVQHMHRQKPPIIHRDLKVENLLLSNQGTIKLCDFGSATTISHYPDYSWSAQRRALVEEEITRNTTPMYRTPEIIDLYSNFPIGEKQDIWALGCILYLLCFRQHPFEDGAKLRIVNGKYSIPPHDTQYTVFHSLIRAMLQVNPEERLSIAEVVHQLQEIAAARNVNPKSPITELLEQNGGYGSATLSRGPPPPVGPAGSGYSGGLALA

SEQ ID NO: 164 - protein, 355 aa, Unknown Organism
  Description of Unknown Organism Mammalian protein sequence
MKKFSRMPKSEGGSGGGAAGGGAGGAGAGAGCGSGGSSVGVRVFAVGRHQVTLEESLAEGGFSTVFLVRTHGGIRCALKRMYVNNMPDLNVCKREITIMKELSGHKNIVGYLDCAVNSISDNVWEVLILMEYCRAGQVVNQMNKKLQTGFTEPEVLQIFCDTCEAVARLHQCKTPIIHRDLKVENILLNDGGNYVLCDFGSATNKFLNPQKDGVNVVEEEIKKYTTLSYRAPEMINLYGGKPITTKADIWALGCLLYKLCFFTLPFGESQVAICDGNFTIPDNSRYSRNIHCLIRFMLEPDPEHRPDIFQVSYFAFKFAKKDCPVSNINKCCKQLLRHGALLTEILLFLQLFLNR

SEQ ID NO: 165 - protein, 322 aa, Unknown Organism
  Description of Unknown Organism Mammalian protein sequence
MEGISNFKTPSKLSEKKKSVLCSTPTINIPASPFMQKLGFGTGVNVYLMKRSPRGLSHSPWAVKKINPICNDHYRSVYQKRLMDEAKILKSLHHPNIVGYRAFTEASDGSLCLAMEYGGEKSLNDLIEERYKASQDPFPAAIILKVALNMARGLKYLHQEKKLLHGDIKSSNVVIKGDFETIKICDVGVSLPLDENMTVTDPEACYIGTEPWKPKEAVEENGVITDKADIFAFGLTLWEMMTLSIPHINLSNDDDDEDKTFDESDFDDEAYYAALGTRPPINMEELDESYQKVIELFSVCTNEDPKDRPSAAHIVEALETDV

SEQ ID NO: 166 - protein, 310 aa, Unknown Organism
  Description of Unknown Organism Mammalian protein sequence
PLRELLRERAEKMGQNLNRIPYKDTFWKGTTRTRPRNGTLNKHSGIDFKQLNFLTKLNENHSGELWKGRWQGNDIVVKVLKVRDWSTRKSRDFNEECPRLRIFSHPNVLPVLGACQSPPAPHPTLITHWMPYGSLYNVLHEGTNFVVDQSQAVKFALDMARGMAFLHTLEPLIPRHALNSRSVMIDEDMTARISMADVKFSFQCPGRMYAPAWVAPEALQKKPEDTNRRSADMWSFAVLLWELVTREVPFADLSNMEIGMKVALEGLRPTIPPGISPHVCKLMKICMNEDPAKRPKFDMIVPILEKMQDK

SEQ ID NO: 167 - protein, 6 aa, Artificial Sequence
  Description of Artificial Sequence Synthetic 6xHis tag
HHHHHH

(19) United States
(12) Patent Application Publication
(10) Pub. No.: US 2005/0164300 A1
(43) Pub. Date: Jul. 28, 2005

(54) Molecular scaffolds for kinase ligand development

(75) Inventors: Dean R. Artis, Kensington, CA (US); Ryan E. Bremer, Oakland, CA (US); Samuel J. Gillette, Oakland, CA (US); Clarence R. Hurt, San Ramon, CA (US); Prabha L. Ibrahim, Mountain View, CA (US); Rebecca L. Zuckerman, Alameda, CA (US)

Correspondence Address: FOLEY & LARDNER, P.O. BOX 80278, SAN DIEGO, CA, 92138-0278, US

(73) Assignee: Plexxikon, Inc.

(21) Appl. No.: 10/941,635

(22) Filed: Sep. 15, 2004

Related U.S. Application Data
(60) Provisional application No. 60/503,277, filed on Sep. 15, 2003.

(51) Int. Cl. G01N 33/53; G06F 19/00; G01N 33/48; G01N 33/50

(52) U.S. Cl. 435/7.1; 702/19

(57) ABSTRACT

Molecular scaffolds for compounds active on protein kinases are described, along with methods for using such scaffolds for kinase ligand development. The use of kinase structural information, exemplified with PIM-1 crystals and structural information can, for example, be used for identifying molecular scaffolds and for developing ligands that bind to and modulate particular kinases.

### CROSS-REFERENCE TO RELATED PATENT APPLICATIONS

This application claims the benefit of Ibrahim et al., U.S. Provisional Appl. 60/503,277, filed Sep. 15, 2003, which is incorporated herein by reference in its entirety, including drawings.

### BACKGROUND OF THE INVENTION

This invention relates to the field of development of ligands for protein kinases.

Examples of protein kinases are the PIM kinases, including PIM-1, PIM-2, and PIM-3. The PIM-1 proto-oncogene was originally identified as a genetic locus frequently activated by the proviral insertion of Moloney murine leukemia virus into mouse T cell lymphomas (Cuypers, H. T., Selten, G., Quint, W., Zijlstra, M., Maandag, E. R., Boelens, W., van Wezenbeek, P., Melief, C., and Berns, A. (1984) Murine leukemia virus-induced T-cell lymphomagenesis: integration of proviruses in a distinct chromosomal region. Cell 37: 141-150). The PIM-1 proto-oncogene has also been implicated in human hematopoietic malignancies with its overexpression frequently detected in human hematopoietic cell lines as well as in fresh tumor cells from patients with leukemia (Nagarajan L, Louie E, Tsujimoto Y, ar-Rushdi A, Huebner K, and Croce C M. (1986) Localization of the human PIM oncogene (PIM) to a region of chromosome 6 involved in translocations in acute leukemias. Proc. Natl. Acad. Sci. USA 83: 2556-2560; Meeker T C, Nagarajan L, ar-Rushdi A, Rovera G, Huebner K, and Croce C M. (1987) Characterization of the human PIM-1 gene: a putative proto-oncogene coding for a tissue specific member of the protein kinase family. Oncogene Res. 1: 87-101; Amson R, Sigaux F, Przedborski S, Flandrin G, Givol D, and Telerman A. (1989). The human proto-oncogene product p33PIM is expressed during fetal hematopoiesis and in diverse leukemias. Proc. Natl. Acad. Sci. USA 86: 8857-8861).

The PIM family of proto-oncogenes in human and mouse now consists of at least three members, that code for highly related serine/threonine specific protein kinases (Saris C J, Domen J, and Berns A. (1991) The PIM-1 oncogene encodes two related protein-serine/threonine kinases by alternative initiation at AUG and CUG. EMBO J. 10: 655-664; Eichmann A, Yuan L, Breant C, Alitalo K, and Koskinen P J. (2000) Developmental expression of PIM kinases suggests functions also outside of the hematopoietic system. Oncogene 19: 1215-1224). The function of these three kinases (PIM-1, PIM-2 and PIM-3) appear to complement each other in mice, as deletion of one of the PIM family protein genes did not result in any severe defects (Laird P W, van der Lugt N M, Clarke A, Domen J, Linders K, McWhir J, Berns A, Hooper M. (1993) In vivo analysis of PIM-1 deficiency. Nucl. Acids Res. 21: 4750-4755). During embryonal development PIM genes are expressed in partially overlapping fashion in cells in both immune and central nervous system as well as in epithelia (Eichmann A, Yuan L, Breant C, Alitalo K, and Koskinen P J. (2000) Developmental expression of PIM kinases suggests functions also outside of the hematopoietic system. Oncogene 19: 1215-1224). PIM-1, the prototypical member of the PIM family is located both in the cytoplasm and nucleus, but its precise role in these two locations has not been fully elucidated.

Transgenic mice with PIM-1 driven by Emu enhancer sequences demonstrated that PIM-1 function as a weak oncogene because by itself it does not lead to tumor formation but does so after a second oncogenic gene become overexpressed. In 75% of the tumors over-expressing PIM-1, the second gene found to be over-expressed is c-myc (van der Houven van Oordt C W, Schouten T G, van Krieken J H, van Dierendonck J H, van der Eb A J, Breuer M L. (1998) X-ray-induced lymphomagenesis in E mu-PIM-1 transgenic mice: an investigation of the co-operating molecular events. Carcinogenesis 19: 847-853). In fact when crosses were made between Emu-PIM transgenic mice and Emu-myc transgenic mice, the combination of genes is so oncogenic that the offsprings die in utero due to pre B cell lymphomas (Verbeek S, van Lohuizen M, van der Valk M, Domen J, Kraal G, and Berns A. (1991) Mice bearing the Emu-myc and Emu-PIM-1 transgenes develop pre-B-cell leukemia prenatally. Mol. Cell. Biol., 11: 1176-1179).

Mice deficient for PIM-1 show normal synaptic transmission and short-term plasticity but failed to consolidate enduring LTP even though PIM-2 and PIM-3 are expressed in the hippocampus (Konietzko U, Kauselmann G, Scafidi J, Staubli U, Mikkers H, Berns A, Schweizer M, Waltereit R, and Kuhl D. (1999) PIM kinase expression is induced by LTP stimulation and required for the consolidation of enduring LTP. EMBO J. 18: 3359-3369).

Various factors are known to enhance the transcription of PIM-1 kinase in mouse and human. PIM-1 closely cooperates with another oncoprotein, c-myc, in triggering intracellular signals leading to both transformation and apoptosis and the selective inhibition of apoptotic signaling pathways leading to Bcl-2 (van Lohuizen M, Verbeek S, Krimpenfort P, Domen J, Saris C, Radaszkiewicz T, and Berns A. (1989) Predisposition to lymphomagenesis in PIM-1 transgenic mice: cooperation with c-myc and N-myc in murine leukemia virus-induced tumors. Cell 56: 673-682; Breuer M L, Cuypers H T, Berns A. (1989). Evidence for the involvement of PIM-2, a new common proviral insertion site, in progression of lymphomas. EMBO J. 8: 743-748; Verbeek S, van Lohuizen M, van der Valk M, Domen J, Kraal G, and Berns A. (1991) Mice bearing the E mu-myc and E mu-PIM-1 transgenes develop pre-B-cell leukemia prenatally. Mol. Cell. Biol. 11: 1176-1179; Shirogane T, Fukada T, Muller J M, Shima D T, Hibi M, and Hirano T. (1999) Synergistic roles for PIM-1 and c-Myc in STAT3-mediated cell cycle progression and antiapoptosis. Immunity, 11: 709-719). PIM-1 kinase is induced by T cell antigen receptor cross linking by cytokines and growth factors and by mitogens including IL2, IL3, IL6, IL9, IL12, IL15, GM-CSF, G-CSF, IFNa, INFg, prolactin, ConA, PMA and anti-CD3 antibodies (Zhu N, Ramirez L M, Lee R L, Magnuson N S, Bishop G A, and Gold M R. (2002) CD40 signaling in B cells regulates the expression of the PIM-1 kinase via the NF-kappa B pathway. J. Immunol. 168: 744-754). PIM-1 expression is rapidly induced after cytokine stimulation and the proliferative response to cytokines is impaired in cells from PIM-1 deficient mice (Domen J, van der Lugt N M, Acton D, Laird P W, Linders K, Berns A. (1993) PIM-1 levels determine the size of early B lymphoid compartments in bone marrow. J. Exp. Med. 178: 1665-1673).

Recently, it has been reported that PIM family of kinases interact with Socs-1 protein, a potent inhibitor of JAK activation thereby playing a major role in signaling down stream of cytokine receptors. The phosphorylation of Socs-1 by PIM family of kinases prolongs the half-life of Socs-1 protein, thus potentiating the inhibitory effect of Socs-1 on JAK-STAT activation (Chen X P, Losman J A, Cowan S, Donahue E, Fay S, Vuong B Q, Nawijn M C, Capece D, Cohan V L, Rothman P. (2002) PIM serine/threonine kinases regulate the stability of Socs-1 protein. Proc. Natl. Acad. Sci. USA 99: 2175-2180.). PIM-1 is expressed during GI/S phase of the cell cycle suggesting that it is involved in cell cycle regulation (Liang H, Hittelman W, Nagarajan L., Ubiquitous expression and cell cycle regulation of the protein kinase PIM-1. (1996) Arch Biochem Biophys. 330: 259-265).). PIM-1 kinase activity and the protein level is increased in CD 40 mediated B cell signaling and this increase in PIM-1 level is mediated through the activation of NF-kB (Zhu et al. 2002. supra). PIM-1 can physically interact with NFATc transcription factors enhancing NFATc dependant transactivation and IL2 production in Jurkat cells (Rainio E M, Sandholm J, Koskinen P J. (2002) Cutting edge: Transcriptional activity of NFATc1 is enhanced by the PIM-1 kinase. J. Immunol. 168: 1524-1527). This indicates a novel phosphorylation dependant regulatory mechanism targeting NFATc through which PIM-1 acts as down stream effector of ras to facilitate IL2 dependant proliferation and survival of lymphoid cells (Id.).

PIM-1 is shown to interact with many other targets. Phosphorylation of Cdc25A phosphatase, a direct transcriptional target of c-myc, increase its phosphatase activity both in-vivo and in-vitro indicating that Cdc25A link PIM-1 and c-myc in cell transformation and apoptosis (Mochizuki T, Kitanaka C, Noguchi K, Muramatsu T, Asai A, and Kuchino Y. (1999) Physical and functional interactions between PIM-1 kinase and Cdc25A phosphatase. Implications for the PIM-1-mediated activation of the c-Myc signaling pathway; J. Biol. Chem. 274: 18659-18666). PIM-1 also phosphorylate PTP-U2S, a tyrosine phosphatase associated with differentiation and apoptosis in myeloid cells, decreasing its phosphatase activity and hence preventing premature onset of apoptosis following PMA-induced differentiation (Wang et al. (2001) Pim-1 negatively regulates the activity of PTP-U2S phosphatase and influences terminal differentiation and apoptosis of monoblastoid leukemia cells. Arch. Biochem. Biophys. 390: 9-18). The phosphorylation of p100, a co-activator of c-myb (Weston, 1999, Reassessing the role of C-MYB in tumorigenesis. Oncogene 18: 3034-3038), by PIM-1 is involved in Ras-dependent regulation of transcription (Leverson J D, Koskinen P J, Orrico F C, Rainio E M, Jalkanen K J, Dash A B, Eisenman R N, and Ness S A. (1998) PIM-1 kinase and p100 cooperate to enhance c-Myb activity. Mol. Cell. 2: 417-425). The phosphorylation of another PIM-1 target, heterochromatin protein 1 (HP 1) has been shown to be involved in transcription repression (Koike N, Maita H, Taira T, Ariga H, Iguchi-Ariga S M. (2000) Identification of heterochromatin protein 1 (HP 1) as a phosphorylation target by PIM-1 kinase and the effect of phosphorylation on the transcriptional repression function of HP-1 (1). FEBS Lett. 467: 17-21).

The information provided above is intended solely to assist the understanding of the reader. None of the information provided or references cited is admitted to be prior art to the present invention.

### SUMMARY OF THE INVENTION

The present invention concerns molecular scaffolds that can be used to identify and develop ligands active on one or more kinases, for example, the PIM kinases, (e.g., PIM-1, PIM-2, and PIM-3). Compounds representing the present molecular scaffolds have been co-crystallized with PIM-1, and the co-crystal structures have been determined to confirm the orientation of the compound within the binding site. In addition, such compounds also bind to other kinases, such that the scaffolds can be used for ligand development for other kinases also. In the description herein, PIM-1 and the use of molecular scaffolds and ligands with PIM-1 are described as examples, but the invention is not limited to PIM-1.

In a first aspect, the invention provides a kinase scaffold library comprising at least one set of compounds of a chemical structure selected from the group consisting of Formula I, II, III, IV, V, VI, and VII as described herein, Formula I, II, and III as described in U.S. application Ser. No. 10/664,421 and corresponding PCT/US03/29415, and Formula I as described in U.S. application Ser. No. 10/789,818 and corresponding PCT/U.S. 2004/005904, all of which are incorporated herein by reference in their entireties. (Unless specifically indicated to the contrary, reference to any of Formulas I-VII means the Formulas I-VII described with a generic structure herein.). In certain embodiments, the scaffold library contains at least one set of compounds having chemical structures of Formula I, II, III, IV, V, VI, or VII.

Libraries with large numbers of compounds can be highly useful. In particular embodiments, a library includes at least 50, 100, 200, 300, 400, 500, 600, 800, 1000 1400, or even more different compounds of the particular chemical structure; a library can include a plurality of such sets of compounds of different chemical structures selected from the indicated Formulas; a plurality of sets of compounds of different chemical structure can include any combination of the specified chemical structures, e.g., Formulas I and II, Formulas I and III, Formulas II and III, Formulas I, II, and III (including each individual combination of the 11 Formulas listed above, taken 2, 3, 4, 5, 6, 7, 8, 9, 10, or 11 at a time); a plurality of sets is 2, 3, 4, 5, 6, 7, 8, 9, 10, or 11, or more such sets; a majority of compounds in a set or sets have been demonstrated to bind to one or more kinases; one or more kinases are selected from the kinases including PIM-1, Pyk2, c-Abl, Her2, cMet, VEGFR, EGFR, cKit, Pkcβ, p38, Cdk2, Akt, Gsk3β, or other kinase listed in Table 5; the kinase domain of the kinase has at least 30%, 35%, 40% or more sequence identity to PIM-1 kinase domain.

As used herein in connection with the present compounds, the term “scaffold library” refers to a defined set of compounds in a format suitable for testing as biochemical or biological activity modulators. For example, such compounds can be in solution or dry in wells of a plate such as a microtiter plate (or a plurality of such plates). Such a library is distinguished from conventional compound libraries and commercially available compound libraries by being selected such that at least 50, 60, 70, 80, 90, 95, 98, or 100% of the compounds in the library are derivatives of the chemical structures that have been described herein as kinase binding compounds or molecular scaffolds. Such libraries can also include compounds that are derivatives of other kinase binding compounds.

Because initial ligand identification can be carried out by fitting electronic representations of compounds in binding sites of target molecules, in another aspect the invention provides a system for fitting compounds in binding sites of one or more protein kinases. Such a system includes an electronic kinase scaffold library that includes at least one set of electronic representations of compounds of a chemical structure selected from the group consisting of Formula I, II, III, IV, V, VI, and VII as described herein, Formula I, II, and III as described in U.S. application Ser. No. 10/664,421 and corresponding PCT/US03/29415, and Formula I as described in U.S. application Ser. No. 10/789,818 and corresponding PCT/U.S. 2004/005904, where the kinase scaffold library is embedded in a computer memory device, the electronic representations of the compounds can be selectively retrieved and functionally connected with computer software adapted to fit electronic representations of compounds in an electronic representation of a binding site of a kinase. The system can also include at least one electronic representation of a kinase binding site (e.g., an electronic representation of a crystal structure of a kinase, kinase domain, or kinase binding site) embedded in computer memory such that the electronic representation of a kinase binding site can be functionally connected with the computer software. The system can include one or more electronic representations of binding sites of kinases selected from PIM-1, Pyk2, c-Abl, Her2, cMet, VEGFR, EGFR, cKit, Pkcβ, p38, Cdk2, Akt, Gsk3β, or another kinase from Table 5, e.g., 1, 2, 3, 4, 5, 6, 7, 8, 9, 10, or more such kinases.

In a related aspect, the invention provides a method for obtaining improved ligands binding to a kinase (e.g., PIM-1, Pyk2, c-Abl, Her2, cMet, VEGFR, EGFR, cKit, Pkcβ, p38, Cdk2, Akt, Gsk3β, or other kinase from Table 5), where the method involves determining whether a derivative of a compound of any of Formulas I, II, III, IV, V, VI, or VII that binds to the kinase, binds to the kinase with greater affinity or greater specificity or both than the parent binding compound. Binding with greater affinity or greater specificity or both than the parent compound indicates that the derivative is an improved ligand. This process can also be carried out in successive rounds of selection and derivatization and/or with multiple parent compounds to provide a compound or compounds with improved ligand characteristics. Likewise, the derivative compounds can be tested and selected to give high selectivity for the kinase, or to give cross-reactivity to a particular set of kinase targets.

In the context of the present invention, the terms “kinase” and “protein kinase” refers to an enzyme that phosphorylates other proteins. These enzymes are often grouped according to the amino acid that is phosphorylated, into tyrosine kinases, serine/threonine kinases, and histidine kinases.

The term “PIM kinase” or “PIM family kinase” means a protein kinase with greater than 45% amino acid sequence identity to PIM-1 from the same species, and includes PIM-1, PIM-2, and PIM-3. Unless clearly indicated to the contrary, use of the term “PIM kinase” constitutes a reference to any of the group of PIM kinases, specifically including individual reference to each of PIM-1, PIM-2, and PIM-3.

As used herein, the terms “ligand” and “modulator” refer to a compound that modulates the activity of a target biomolecule, e.g., an enzyme such as a kinase. Generally a ligand or modulator will be a small molecule, where “small molecule refers to a compound with a molecular weight of 1500 daltons or less, or preferably 1000 daltons or less, 800 daltons or less, or 600 daltons or less. Thus, an “improved ligand” is one that possesses better pharmacological and/or pharmacokinetic properties than a reference compound, where “better” can be defined by a person for a particular biological system or therapeutic use.

In the context of binding compounds, molecular scaffolds, and ligands, the term “derivative” or “derivative compound” refers to a compound having a chemical structure that contains a common core chemical structure as a parent or reference compound, but differs by having at least one structural difference, e.g., by having one or more substituents added and/or removed and/or substituted, and/or by having one or more atoms substituted with different atoms. Unless clearly indicated to the contrary, the term “derivative” does not mean that the derivative is synthesized using the parent compound as a starting material or as an intermediate, although in some cases, the derivative may be synthesized from the parent.

Thus, the term “parent compound” refers to a reference compound for another compound, having structural features continued in the derivative compound. Often but not always, a parent compound has a simple chemical structure than the derivative.

By “chemical structure” or “chemical substructure” is meant any definable atom or group of atoms that constitute a part of a molecule. Normally, chemical substructures of a scaffold or ligand can have a role in binding of the scaffold or ligand to a target molecule, or can influence the three-dimensional shape, electrostatic charge, and/or conformational properties of the scaffold or ligand.

The term “binds” in connection with the interaction between a target and a potential binding compound indicates that the potential binding compound associates with the target to a statistically significant degree as compared to association with proteins generally (i.e., non-specific binding). Thus, the term “binding compound” refers to a compound that has a statistically significant association with a target molecule. Preferably a binding compound interacts with a specified target with a dissociation constant (k_{d}) of 1 mM or less. A binding compound can bind with “low affinity”, “very low affinity”, “extremely low affinity”, “moderate affinity”, “moderately high affinity”, or “high affinity” as described herein.

In the context of compounds binding to a target, the term “greater affinity” indicates that the compound binds more tightly than a reference compound, or than the same compound in a reference condition, i.e., with a lower dissociation constant. In particular embodiments, the greater affinity is at least 2, 3, 4, 5, 8, 10, 50, 100, 200, 400, 500, 1000, or 10,000-fold greater affinity.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., other kinases or even other type of enzymes. In particular embodiments, the greater specificity is at least 2, 3, 4, 5, 8, 10, 50, 100, 200, 400, 500, or 1000-fold greater specificity.

As used in connection with binding of a compound with a particular kinase, the term “interact” indicates that the distance from a bound compound to a particular amino acid residue will be 5.0 angstroms or less, or 6 angstroms or less with one water molecule coordinated between the compound and the residue, or 9 angstroms or less with two water molecules coordinated between the compound and the residue. In particular embodiments, the distance from the compound to the particular amino acid residue is 4.5 angstroms or less, 4.0 angstroms or less, or 3.5 angstroms or less. Such distances can be determined, for example, using co-crystallography, or estimated using computer fitting of a compound in the kinase active site.

Reference to particular amino acid residues in PIM-1 polypeptide residue number is defined by the numbering provided in Meeker, T. C., Nagarajan, L., ar-Rushdi, A., Rovera, G., Huebner, K., Corce, C. M.; (1987) Characterization of the human PIM-1 gene: a putative proto-oncogene coding for a tissue specific member of the protein kinase family. Oncogene Res. 1: 87-101, in accordance with the sequence provided in SEQ ID NO: 1. PIM-2 is as described in Baytel et al. (1998) The human Pim-2 proto-oncogene and its testicular expression, Biochim. Biophys. Acta 1442,274-285. PIM-3 from rat is described in Feldman, et al. (1998) KID-1, a protein kinase induced by depolarization in brain, J. Biol. Chem. 273, 16535-16543; and Kinietzko et al. (1999) Pim kinase expression is induced by LTP stimulation and required for the consolidation of enduring LTP, EMBO J. 18, 3359-3369. (KID-1 is the same as PIM-3.) Human PIM-3 nucleic acid and amino acid sequences are provided herein.

In a related aspect, the invention provides a method for developing ligands specific for a kinase, such as a PIM kinase, e.g., PIM-1, where the method involves determining whether a derivative of a compound that binds to a plurality of kinases has greater specificity for the particular kinase than the parent compound.

As used herein in connection with binding compounds or ligands, the term specific for a kinase”, “specific for PIM-1” and terms of like import mean that a particular compound binds to the particular kinase to a statistically greater extent than to other kinases that may be present in a particular organism. Also, where biological activity other than binding is indicated, the term “specific for a kinase” indicates that a particular compound has greater biological activity associated with binding to the particular kinase than to other kinases. Preferably, the specificity is also with respect to other biomolecules (not limited to kinases) that may be present from an organism. A particular compound may also be selected that is specific for kinase sub-group (e.g., tyrosine kinases, serine/threonine kinases, histidine kinases), indicating that it binds to and/or has a greater biological activity associated with binding to a plurality of kinases in that sub-group than to other kinases.

In another aspect, the invention concerns a method for developing ligands binding to a particular kinase, e.g., PIM-1, where the method includes determining the orientation of at least one molecular scaffold of Formula I, II, III, IV, V, VI, or VII in co-crystals with the kinase; identifying chemical structures of one or more of the molecular scaffolds, that, when modified, alter the binding affinity or binding specificity or both between the molecular scaffold and the kinase; and synthesizing a ligand in which one or more of the chemical structures of the molecular scaffold is modified to provide a ligand that binds to the kinase with altered binding affinity or binding specificity or both. Due to the significant of sequence identity between various kinases, e.g., PIM-1 and the other PIM kinases, PIM-1 can also be used as a surrogate or in a homology model for orientation determination and to allow identification of chemical structures that can be modified to provide improved ligands.

By “molecular scaffold” is meant a core molecule to which one or more additional chemical moieties can be covalently attached, modified, or eliminated to form a plurality of molecules with common structural elements. The moieties can include, but are not limited to, a halogen atom, a hydroxyl group, a methyl group, a nitro group, a carboxyl group, or any other type of molecular group including, but not limited to, those recited in this application. Molecular scaffolds bind to at least one target molecule, and the target molecule can preferably be a protein or enzyme. Preferred characteristics of a scaffold can include binding at a target molecule binding site such that one or more substituents on the scaffold are situated in binding pockets in the target molecule binding site; having chemically tractable structures that can be chemically modified, particularly by synthetic reactions, so that a combinatorial library can be easily constructed; having chemical positions where moieties can be attached that do not interfere with binding of the scaffold to a protein binding site, such that the scaffold or library members can be modified to achieve additional desirable characteristics, e.g., enabling the ligand to be actively transported into cells and/or to specific organs, or enabling the ligand to be attached to a chromatography column for additional analysis.

By “binding site” is meant an area of a target molecule to which a ligand can bind non-covalently. Binding sites embody particular shapes and often contain multiple binding pockets present within the binding site. The particular shapes are often conserved within a class of molecules, such as a molecular family. Binding sites within a class also can contain conserved structures such as, for example, chemical moieties, the presence of a binding pocket, and/or an electrostatic charge at the binding site or some portion of the binding site, all of which can influence the shape of the binding site.

By “binding pocket” is meant a specific volume within a binding site. A binding pocket can often be a particular shape, indentation, or cavity in the binding site. Binding pockets can contain particular chemical groups or structures that are important in the non-covalent binding of another molecule such as, for example, groups that contribute to ionic, hydrogen bonding, or van der Waals interactions between the molecules.

By “orientation”, in reference to a binding compound bound to a target molecule is meant the spatial relationship of the binding compound and at least some of its constituent atoms to the binding pocket and/or atoms of the target molecule at least partially defining the binding pocket.

By “co-crystals” is meant a complex of the compound, molecular scaffold, or ligand bound non-covalently to the target molecule and present in a crystal form appropriate for analysis by X-ray or protein crystallography. In preferred embodiments the target molecule-ligand complex can be a protein-ligand complex.

The phrase “alter the binding affinity or binding specificity” refers to changing the binding constant of a first compound for another, or changing the level of binding of a first compound for a second compound as compared to the level of binding of the first compound for third compounds, respectively. For example, the binding specificity of a compound for a particular protein is increased if the relative level of binding to that particular protein is increased as compared to binding of the compound to unrelated proteins.

As used herein in connection with test compounds, binding compounds, and modulators (ligands), the term “synthesizing” and like terms means chemical synthesis from one or more precursor materials.

The phrase “chemical structure of the molecular scaffold is modified” means that a derivative molecule has a chemical structure that differs from that of the molecular scaffold but still contains common core chemical structural features. The phrase does not necessarily mean that the molecular scaffold is used as a precursor in the synthesis of the derivative.

By “assaying” is meant the creation of experimental conditions and the gathering of data regarding a particular result of the experimental conditions. For example, enzymes can be assayed based on their ability to act upon a detectable substrate. A compound or ligand can be assayed based on its ability to bind to a particular target molecule or molecules.

Certain compounds have been identified as molecular scaffolds and binding compounds for protein kinases, as exemplified by PIM-1. Thus, in another aspect, the invention provides a method for identifying a ligand binding to specific kinase, that includes determining whether a derivative compound that includes a core structure selected from the group consisting of the core structures of Formula I, Formula II, Formula III, Formula IV, Formula V, Formula VI, and Formula VII, as described herein binds to the kinase with altered binding affinity or specificity or both as compared to a parent compound.

In reference to compounds of any of Formula I, II, III, IV, V, VI, and VII, the term “core structure” refers to the structures shown diagramatically as part of the description of compounds of each of Formulas I-VII, but excluding non-ring variable substituents. More generally, the term “core structure” refers to a characteristic chemical structure common to a set of compounds, especially a chemical structure that carries variable substituents in the compound set.

By a “set” of compounds is meant a collection of compounds. The compounds may or may not be structurally related.

The invention further concerns co-crystals of a particular kinase and a kinase binding compound of Formula I, II, III, IV, V, VI, or VII. Advantageously, such co-crystals are of sufficient size and quality to allow structural determination to at least 3 Angstroms, 2.5 Angstroms, or 2.0 Angstroms. The co-crystals can, for example, be in a crystallography plate, be mounted for X-ray crystallography and/or in an X-ray beam. Such co-crystals are beneficial, for example, for obtaining structural information concerning interaction between the kinase and kinase binding compounds.

Kinase binding compounds can include compounds that interact with at least one of PIM-1 residues 49, 52, 65, 67, 121, 128, and 186, or any 2, 3, 4, 5, 6, or 7 of those residues. Exemplary compounds that bind to PIM-1 include compounds of any of Formulas I-VII.

Likewise, in additional aspects, methods for obtaining PIM-1 co-crystals with compounds of Formula I, II, III, IV, V, VI, and VII are provided. The method involves subjecting PIM-1 protein at 5-20 mg/ml to crystallization conditions substantially equivalent to Hampton Screen 1 conditions 2, 7, 14, 17, 23, 25, 29, 36, 44, or 49, in the presence of binding compound for a time sufficient for crystal development. The binding compound may be added at various concentrations depending on the nature of the compound, e.g., final concentration of 0.5 to 1.0 mM. In many cases, the binding compound will be in an organic solvent such as dimethyl sulfoxide solution. Exemplary co-crystallization conditions include 0.4-0.9 M sodium acetate trihydrate pH 6.5, 0.1 M imidazole; or 0.2-0.7 M sodium potassium tartrate, 00.1 M MES buffer pH 6.5.

In another aspect, provision of compounds active on a variety of different kinases (e.g., PIM-1) also provides a method for modulating kinase activity by contacting the kinase with a compound of any of Formulas I, II, III, IV, V, VI, and VII that binds to the kinase. The compound is preferably provided at a level sufficient to modulate the activity of PIM-1 by at least 10%, more preferably at least 20%, 30%, 40%, or 50%. In many embodiments, the compound will be at a concentration of about 1 EM, 100 μM, or 1 mM, or in a range of 1-100 nM, 100-500 nM, 500-1000 nM, 1-100 μM, 100-500 μM, or 500-1000 μM. The compound can be one that interacts with one more of PIM-1 residues 49, 52, 65, 67, 121, 128, and 186.

As used herein, the term “modulating” or “modulate” refers to an effect of altering a biological activity, especially a biological activity associated with a particular biomolecule such as PIM-1. For example, an agonist or antagonist of a particular biomolecule modulates the activity of that biomolecule, e.g., an enzyme.

The term “PIM-1 activity” refers to a biochemical activity of PIM-1, particularly including kinase activity.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction (e.g., modulating enzymatic action) between the compound and other specified material can occur.

In a related aspect, the invention provides a method for treating a patient suffering from or at risk of a kinase-mediated disease or condition or a disease or condition in which kinase modulation provides a therapeutic benefit, such as a disease or condition characterized by abnormal kinase activity, e.g., PIM-1 activity, where the method involves administering to the patient a compound of Formula I, II, III, IV, V, VI, or VII. The compound can, for example, be one that interacts with one or more of PIM-1 residues 49, 52, 65, 67, 121, 128, and 186.

In certain embodiments, the disease or condition is a proliferative disease or neoplasia, such as benign or malignant tumors, psoriasis, leukemias (such as myeloblastic leukemia), lymphoma, prostate cancer, liver cancer, breast cancer, sarcoma, neuroblastoma, Wilm's tumor, bladder cancer, thyroid cancer, neoplasias of the epithelial origin such as mammacarcinoma, or a chronic inflammatory disease or condition, resulting, for example, from a persistent infection (e.g., tuberculosis, syphilis, fungal infection), from prolonged exposure to endogenous (e.g., elevated plasma lipids) or exogenous (e.g., silica, asbestos, cigarette tar, surgical sutures) toxins, and from autoimmune reactions (e.g., rheumatoid arthritis, systemic lupus erythrymatosis, multiple sclerosis, psoriasis). Thus, chronic inflammatory diseases include many common medical conditions, such as rheumatoid arthritis, restenosis, psoriasis, multiple sclerosis, surgical adhesions, tuberculosis, and chronic inflammatory lung and airway diseases, such as asthma pheumoconiosis, chronic obstructive pulmonary disease, nasal polyps, and pulmonary fibrosis. Kinase modulators may also be useful in inhibiting development of hematomous plaque and restinosis, in controlling restinosis, as anti-metastatic agents, in treating diabetic complications, as immunosuppressants, and in control of angiogenesis to the extent the kinase is involved in a particular disease or condition.

In certain embodiments, the disease or condition is one listed in Table 5; the disease or condition is one listed in Table 5. In certain embodiments, the therapeutic or prophylactic effect of the compound is due to modulation of a kinase from Table 5; the therapeutic or prophylactic effect of the compound is due to modulation of a kinase from Table 5 and the disease or condition is one that corresponds thereto in Table 5.

As used herein, the term “kinase-mediated” disease or condition and like terms refer to a disease or condition in which the biological function of a kinase affects the development and/or course of the disease or condition, and/or in which modulation of a kinase alters the development, course, and/or symptoms of the disease or condition. Similarly, the phrase “kinase modulation provides a therapeutic benefit” indicates that modulation of the level of activity of a kinase in a subject indicates that such modulation reduces the severity and/or duration of the disease, reduces the likelihood or delays the onset of the disease or condition, and/or causes an improvement in one or more symptoms of the disease or condtion. Parallel terms apply to each of the kinases indicated herein.

Because molecular scaffolds are described, a large number of different kinases can be used in connection with the described scaffolds and compounds. A list of such kinases (not intended to be comprehensive) is provided in Table 6. Exemplary kinases and a major indication for which modulation of the kinase is useful include the following:

TABLE 6

| | Indication |
|---|---|
| Tyrosine Kinases | |
| c-abl | CML (chronic myeloid leukemia) |
| her2 | breast cancer |
| c-met | cancer |
| VEGFR | angiogenesis |
| c-kit | cancer |
| Serine Kinases | |
| pkcβ | retinopathy |
| p38 | inflammation/RA |
| cdk2 | cancer |
| akt | cancer/apoptosis |
| gsk3β | diabetes |

As crystals of PIM-1 and other kinases have been developed and analyzed, another aspect concerns an electronic representation of the kinase with an electronic representation of a kinase binding compound or a test compound in the binding site, where the compound has a chemical structure of Formula I, Formula II, Formula III, Formula IV, Formula V, Formula VI, or Formula VII.

Likewise, in a related aspect, the invention concerns an electronic representation of a portion of a kinase binding site, e.g., PIM-1, (which can be an active site), which includes a representation of Formula I, II, III, IV, V, VI, or VII. A binding site can be represented in various ways, e.g., as representations of atomic coordinates of residues around the binding site and/or as a binding site surface contour, and can include representations of the binding character of particular residues at the binding site, e.g., conserved residues.

In another aspect, the invention provides a method for identifying potential kinase, e.g., PIM-1 or other kinase listed herein, binding compounds by fitting at least one electronic representation of a compound of Formula I, II, III, IV, V, VI, or VII in an electronic representation of a kinase, e.g., PIM-1, binding site. The representation of the binding site may be part of an electronic representation of a larger portion(s) (e.g., kinase domain) or all of a PIM molecule or may be a representation of only the binding site. The electronic representation may be as described above or otherwise described herein.

In particular embodiments, the method involves fitting a computer representation of a compound from a computer database with a computer representation of the active site of a kinase, e.g., PIM-1; and involves removing a computer representation of a compound complexed with the kinase molecule and identifying compounds that best fit the active site based on favorable geometric fit and energetically favorable complementary interactions as potential binding compounds.

In other embodiments, the method involves modifying a computer representation of a compound complexed with a kinase molecule, e.g., PIM-1, by the deletion or addition or both of one or more chemical groups; fitting a computer representation of a compound from a computer database with a computer representation of the active site of the kinase molecule; and identifying compounds that best fit the active site based on favorable geometric fit and energetically favorable complementary interactions as potential binding compounds.

In still other embodiments, the method involves removing a computer representation of a compound complexed with a kinase, such as PIM-1, and searching a database for compounds having structural similarity to the complexed compound using a compound searching computer program or replacing portions of the complexed compound with similar chemical structures using a compound construction computer program.

Fitting a compound can include determining whether a compound will interact with one or more of PIM-1 residues 49, 52, 65, 67, 121, 128, and 186.

In another aspect, the invention concerns a method for attaching a kinase binding compound of Formula I, II, III, IV, V, VI, or VII (e.g., a PIM-1 binding compound) to an attachment component, as well as a method for identifying attachment sites on such kinase binding compound. The method involves identifying energetically allowed sites for attachment of an attachment component; and attaching the compound or a derivative thereof to the attachment component at the energetically allowed site. In certain embodiments, the kinase is a kinase listed herein; the kinase has at least 25% amino acid sequence identity or 30% sequence similarity to wild type PIM-1, and/or includes conserved residues matching at least one of PIM-1 residues 49, 52, 65, 67, 121, 128, and 186 (i.e., matching any one, any 2, 3, 4, 5, 6, or 7 of those residues).

Attachment components can include, for example, linkers (including traceless linkers) for attachment to a solid phase or to another molecule or other moiety. Such attachment can be formed by synthesizing the compound or derivative on the linker attached to a solid phase medium e.g., in a combinatorial synthesis in a plurality of compound. Likewise, the attachment to a solid phase medium can provide an affinity medium (e.g., for affinity chromatography).

The attachment component can also include a label, which can be a directly detectable label such as a fluorophore, or an indirectly detectable such as a member of a specific binding pair, e.g., biotin.

The ability to identify energetically allowed sites on a kinase binding compound of Formula I, II, III, IV, V, VI, and VII, e.g., a PIM-1 binding compound also, in a related aspect, provides modified binding compounds that have linkers attached, for example, compounds of Formula I-VII, preferably at an energetically allowed site for binding of the modified compound to a kinase. The linker can be attached to an attachment component as described above.

Still another aspect of the invention concerns a method for developing a ligand for a kinase that includes conserved residues matching any one, 2, 3, 4, 5, 6, or 7 of PIM-1 residues 49, 52, 65, 67, 121, 128, and 186, by determining whether a compound of Formula I, II, III, IV, V, VI, or VII binds to the kinase. The method can also include determining whether the compound modulates the activity of the kinase. In certain embodiments, the kinase has at least 25% sequence identity or at least 30% sequence similarity to PIM-1, or PIM-1 kinase domain.

In particular embodiments, the determining includes computer fitting the compound in a binding site of the kinase and/or the method includes forming a co-crystal of the kinase and the compound. Such co-crystals can be used for determining the binding orientation of the compound with the kinase and/or provide structural information on the kinase, e.g., on the binding site and interacting amino acid residues. Such binding orientation and/or other structural information can be accomplished using X-ray crystallography.

The invention also provides compounds of Formula I, II, III, IV, V, VI, and VII that bind to and/or modulate (e.g., inhibit) kinase activity for a particular kinase, e.g., PIM-1. Accordingly, in aspects and embodiments involving kinase binding compounds, molecular scaffolds, and ligands or modulators, the compound is a weak binding compound; a moderate binding compound; a strong binding compound; a compound that binds at a level identified herein; the compound interacts with one or more of PIM-1 residues 49, 52, 65, 67, 121, 128, and 186; the compound is a small molecule; the compound binds to a plurality of different kinases (e.g., at least 5, 10, 15, 20 different kinases). In particular embodiments, the invention concerns compounds of Formula I, Formula II, Formula III, Formula IV, Formula V, Formula VI, and Formula VII as described below.

In certain embodiments, the invention concerns compounds of Formula I: 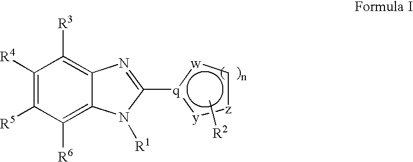
where, with reference to Formula I:

R¹ is hydrogen, optionally substituted lower alkyl, optionally substituted lower alkenyl, optionally substituted lower alkynyl, optionally substituted cycloalkyl, optionally substituted heterocyclyoalkyl, optionally substituted aryl, optionally substituted aralkyl, optionally substituted heteroaryl, optionally substituted heteroaralkyl, —C(X)R²⁰, —C(X)NR¹⁶R¹⁷, —S(O)₂R²¹, or —S(O)₂NR¹⁶R¹⁷.

R² is hydrogen, halo, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)NR¹⁶R¹⁷, —NR²²R²³, or —S(O)₂R²¹, or —S(O)₂NR¹⁶R¹⁷, with the proviso that if R² is attached to nitrogen, it is not —NR²²R²³.

R³, R⁴, R⁵ and R⁶ are independently hydrogen, halo, hydroxy, optionally substituted alkoxyl, optionally substituted thioalkoxy, optionally substituted amine, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl, —C(X)R²⁰, C(X)NR¹⁶R¹⁷, S(O)₂NR¹⁶R¹⁷, —NR²²R²³, or —S(O)₂R²¹;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or R¹⁶ and R¹⁷ together form a 5-7 membered carbocyclic or heterocyclic ring;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R²¹ is optionally substituted lower alkoxy, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R²² and R²³ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, C(X)NR¹⁶R¹⁷, or —S(O)₂R²¹;

w, y, and z are independently O, S, N, or CR²;

q is N or C;

X=O or S; and

n=1 or 2.

In particular embodiments of compounds of Formula I, with reference to Formula I, one or more of R¹, R², R³, R⁴, R⁵, R⁶ is H; any two of R¹, R², R³, R⁴, R⁵, R⁶ are H; any 3 of R¹, R², R³, R⁴, R⁵, R⁶ are H; any 4 of R¹, R², R³, R⁴, R⁵, R⁶ are H; any 5 of R¹, R², R³, R⁴, R⁵, R⁶ are H. Specification of the preceding subgroups is intended to expressly include each possible combination of the specified substituent groups. For example, in particular embodiments, R³, R⁴, R⁵, and R⁶ are H.

Likewise, in certain embodiments the invention concerns compounds of Formula II. 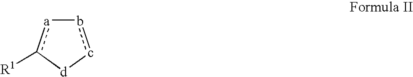
Where, with reference to Formula II:

R¹ is hydrogen, halo, hydroxy, optionally substituted alkoxyl, optionally substituted thioalkoxy, optionally substituted amine, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl, —C(X)R², C(X)NR³R⁴, S(O)₂NR³R⁴, —NR³R⁴, or —S(O)₂R⁵;

a, b, c, and d are independently O, S, NR³, or CR¹¹ with the proviso that two of them are N (and not more than 2) and one (and not more than one) of them is either O or S, and the remaining one is CR¹¹;

R² is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R³ and R⁴ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R⁵ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R¹¹ is hydroxy, optionally substituted alkoxyl, optionally substituted thioalkoxy, optionally substituted amine, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl, —C(X)R², C(X)NR³R⁴, S(O)₂NR³R⁴, —NR³R⁴, or —S(O)₂R⁵;

In particular embodiments of compounds of Formula II, with reference to Formula II, a & b, a & c, a& d, b & c, b & d, or c & d are N. In embodiments where a & b are N, c is S or O, or d is S or O; where a & c are N, b is S or O, or d is S or O; where a & d are N, b is S or O, or c is S or O; where b & c are N, a is S or O, or d is S or O; where b & d are N, a is S or O, or c is S or O, where c &d are N, a is S or O, or b is S or O. In particular embodiments of each of the preceding designations of a, b, c, and d, R¹¹ is optionally substituted hydrogen or halo; R¹¹ is trifluoromethyl, hydroxy, optionally substituted alkoxyl, optionally substituted thioalkoxy; R¹¹ is optionally substituted amine; R¹¹ is optionally substituted lower alkyl, optionally substituted lower alkenyl, optionally substituted lower alkynyl; R¹¹ is optionally substituted cycloalkyl, optionally substituted heterocycloalkyl, optionally substituted aryl, optionally substituted aralkyl, optionally substituted heteroaryl, or optionally substituted heteroaralkyl; R¹¹ is —C(X)R², C(X)NR³R⁴, S(O)₂NR³R⁴, —NR³R⁴, or —S(O)₂R⁵.

In other embodiments, the invention concerns compounds of Formula III. Formula III 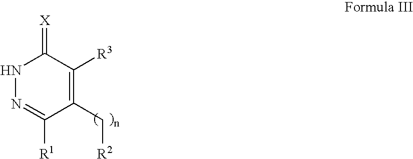
where, with reference to Formula III:

R¹, R², and R³ are independently hydrogen, halo, hydroxy, optionally substituted alkoxyl, optionally substituted thioalkoxy, optionally substituted amine, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl, —C(X)R⁴, —C(X)NR⁵R⁶, —S(O)₂NR⁵R⁶, —NR⁵R⁶, or —S(O)₂R⁷;

R⁴ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R⁵ and R⁶ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or R⁵ and R⁶ together for a 5-7 membered carbocyclic or heterocyclic ring;

R⁷ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

X=O or S; and n=0 or 1.

In particular embodiments of compounds of Formula III, with reference to Formula III, X is O; X is S. In particular embodiments for each selection of X, R¹ is H; R² is H; R³ is H; R¹ and R² are H; R² and R³ are H. In particular embodiments for each selection of X with each selection where one of R¹, R², and R³ is H, the other two of R¹, R² and R³ are independently halo, trifluoromethyl; the other two of R¹, R² and R³ are independently hydroxy, optionally substituted alkoxyl, optionally substituted thioalkoxy; the other two of R¹, R² and R³ are independently optionally substituted amine; the other two of R¹, R² and R³ are independently optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl; the other two of R¹, R² and R³ are independently —C(X)R⁴, —C(X)NR⁵R⁶, —S(O)₂NR⁵R⁶, —NR⁵R⁶, or —S(O)₂R⁷. In particular embodiments, one of R¹, R², and R³ is halo, trifluoromethyl; one of R¹, R², and R³ is hydroxy, optionally substituted alkoxyl, optionally substituted thioalkoxy; one of R¹, R², and R³ is optionally substituted amine; one of R¹, R², and R³ is optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl one of R¹, R², and R³ is C(X)R⁴, —C(X)NR⁵R⁶, —S(O)₂NR⁵R⁶, —NR⁵R⁶, or —S(O)₂R⁷.

In other embodiments, the invention concerns compounds of Formula IV. 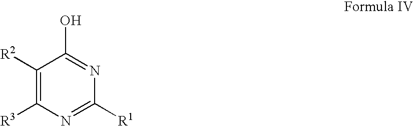
where, with reference to Formula IV:

R¹ is optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —NR¹⁶R¹⁷, —OR²¹, or —SR²¹;

R² and R³ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, optionally substituted heteroaralkyl, —C(X)R²⁰, —C(X)NR¹⁶R¹⁷, —C(X)R²⁰, or —C(X)NR¹⁶R¹⁷;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or R¹⁶ and R¹⁷ together form a carbocyclic or heterocyclic ring;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl; and

R²¹ is optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl.

In particular embodiments of compounds of Formula IV, with reference to Formula IV, R¹ is optionally substituted lower alkyl; R¹ is optionally substituted lower alkenyl; R¹ is optionally substituted lower alkynyl, optionally substituted cycloalkyl; R¹ is optionally substituted heterocycloalkyl; R¹ is optionally substituted aryl, optionally substituted aralkyl; R¹ is optionally substituted heteroaryl, optionally substituted heteroaralkyl; R¹ is —NR¹⁶R¹⁷, —OR²¹, —SR²¹, —C(X)R²⁰, or —C(X)NR¹⁶R¹⁷. In particular embodiments, R² or R³ (but not both) is hydrogen; R² or R³ (but not the other) is optionally substituted lower alkyl, optionally substituted lower alkenyl, optionally substituted lower alkynyl, optionally substituted cycloalkyl; R² or R³ (but not the other) is optionally substituted heterocycloalkyl; R² or R³ (but not the other) is optionally substituted aryl, optionally substituted aralkyl; R² or R³ (but not the other) is optionally substituted heteroaryl, optionally substituted heteroaralkyl, or optionally substituted heteroaralkyl; R² or R³ (but not both) is —C(X)R²⁰, or —C(X)NR¹⁶R¹⁷. In particular embodiments, R² is H; R³ is H; R² and R³ are H.

The invention also concerns compounds of Formula V. 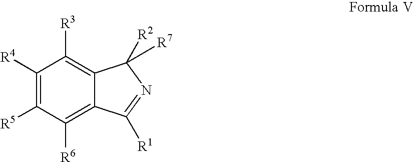
where, with reference to Formula V:

R¹ and R⁷ are independently hydrogen, hydroxyl, optionally substituted alkoxyl, optionally substituted thioalkoxy, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —NR¹⁶R¹⁷, —C(X)R²⁰, C(X)NR¹⁶R¹⁷, —S(O)₂NR¹⁶R¹⁷, —S(O)₂R²¹, or R¹ and R⁷, when one of them is —NR¹⁶R¹⁷, hydroxyl, alkoxyl, thioalkoxyl, aralkyl or heteroaralkyl and the other one is hydrogen can combine to form ═NR¹⁶, ═O, ═S, or ═Caryl/heteroaryl, with the proviso that R¹ and R⁷ both cannot be hydroxyl, alkoxyl, thioalkoxyl or —NR¹⁶R¹⁷ at the same time;

R² is hydrogen, halo, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, optionally substituted heteroaralkyl, —C(X)R²⁰, or C(X)NR¹⁶R¹⁷;

R³, R⁴, R⁵, and R⁶ are independently hydrogen, halo, hydroxyl, optionally substituted alkoxyl, optionally substituted thioalkoxy, optionally substituted amine, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, —C(X)NR¹⁶R¹⁷, —S(1)₂NR¹⁶R¹⁷, or —S(O)₂R²¹;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or R¹⁶ and R¹⁷ together form a carbocyclic or heterocyclic ring;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl; and

R²¹ is optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl.

In particular embodiments of compounds of Formula V, with reference to Formula V, R¹ is H; R² is H; R³ is H; R⁴ is H; R⁵ is H; R⁶ is H; each combination of two of R¹, R², R³, R⁴, R, and R⁶ are H and the others; each combination of three of R¹, R², R³, R⁴, R⁵, and R⁶ are H; each combination of 4 of R¹, R², R³, R⁴, R⁵, and R⁶ are H; each combination of 5 of R¹, R³, R⁴, R⁵, and R⁶ are H.

Likewise, the invention concerns compounds of Formula VI. 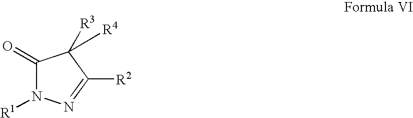
where, with reference to Formula VI:

R¹ is hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, —C(X)NR¹⁶R¹⁷, —S(O)₂NR¹⁶R¹⁷, or —S(O)₂R²¹;

R² is hydrogen, halo, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, optionally substituted heteroaralkyl, —C(X)R²⁰, —C(X)NR¹⁶R¹⁷, —S(O)₂NR¹⁶R¹⁷, or —S(O)₂R²¹;

R³ and R⁴ are independently hydrogen, halo, hydroxyl, optionally substituted alkoxyl, optionally substituted thioalkoxy, optionally substituted amine, optionally substituted lower alkyl (e.g., trifluoromethyl), optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —NR¹⁶R^{17,} —C(X)R²⁰, —C(X)NR¹⁶R¹⁷, —S(O)₂NR¹⁶R¹⁷, or —S(O)₂R²¹ or R³ and R⁴, when one of them is —NR¹⁶R¹⁷, hydroxyl, alkoxyl, thioalkoxyl, aralkyl or heteroaralkyl and the other one is hydrogen can combine to form ═NR¹⁶, ═O, ═S, or ═Caryl/heteroaryl, with the proviso that R¹ and R⁷ both cannot be hydroxyl, alkoxyl, thioalkoxyl or —NR¹⁶R¹⁷ at the same time;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or R¹⁶ and R¹⁷ together form a carbocyclic or heterocyclic ring;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl; and

R²¹ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl.

In particular embodiments of compounds of Formula VI, with reference to Formula VI, R¹ is H; R² is H; R³ is H; R¹ and R² are H; R² and R³ are H; R¹ is optionally substituted lower alkyl, optionally substituted lower alkenyl, optionally substituted lower alkynyl; R¹ is optionally substituted cycloalkyl, optionally substituted heterocycloalkyl; R¹ is optionally substituted aryl, optionally substituted aralkyl, optionally substituted heteroaryl, optionally substituted heteroaralkyl; R¹ is —C(X)R²⁰, —C(X)NR¹⁶R⁷, —S(O)₂NR¹⁶R¹⁷, or —S(O)₂R²¹; R² is halo; R² is optionally substituted lower alkyl, optionally substituted lower alkenyl, optionally substituted lower alkynyl; R² is optionally substituted cycloalkyl, optionally substituted heterocycloalkyl; R² is optionally substituted aryl, optionally substituted aralkyl, optionally substituted heteroaryl, optionally substituted heteroaralkyl, or optionally substituted heteroaralkyl; R² is —C(X)R²⁰, —C(X)NR¹⁶R¹⁷, —S(O)₂NR¹⁶R¹⁷, or —S(O)₂R²¹; R³ is halo; R³ is trifluoromethyl; R³ is optionally substituted alkoxyl, optionally substituted thioalkoxy; R³ is optionally substituted amine; R³ is optionally substituted lower alkyl, optionally substituted lower alkenyl, optionally substituted lower alkynyl; R³ is optionally substituted cycloalkyl, optionally substituted heterocycloalkyl; R³ is optionally substituted aryl, optionally substituted aralkyl, optionally substituted heteroaryl, or optionally substituted heteroaralkyl; R³ is —C(X)R²⁰, —C(X)NR¹⁶R¹⁷, —S(O)₂NR¹⁶R¹⁷, or —S(O)₂R²¹.

In yet other embodiments, the invention concerns compounds of Formula VII. 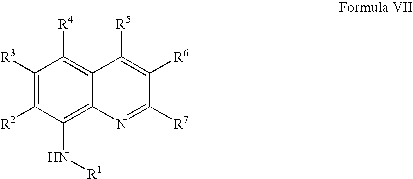
where, with reference to Formula VII:

R¹ is hydrogen, optionally substituted lower alkyl, optionally substituted lower alkenyl, optionally substituted lower alkynyl, optionally substituted cycloalkyl, optionally substituted heteralkyl, optionally substituted heterocycloalkyl, optionally substituted aryl, optionally substituted aralkyl, optionally substituted heteroaryl, optionally substituted heteroaralkyl, —C(X)R²⁰, —C(X)NR¹⁶R¹⁷, —S(O)₂NR¹⁶R¹⁷, or —S(O)₂R²¹;

R², R³, R⁴, R⁵, R⁶, and R⁷ are independently hydrogen, halo, optionally substituted amine, optionally substituted alkoxy, optionally substituted thioether, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, optionally substituted heteroaralkyl, —C(X)R²⁰, —C(X)NR¹⁶R¹⁷, —S(O)₂NR¹⁶R¹⁷, or —S(O)₂R²¹;

R¹⁶ and R¹⁷ are independently hydrogen, optionally substituted lower alkyl, optionally substituted lower alkenyl, optionally substituted lower alkynyl, optionally substituted cycloalkyl, optionally substituted heteroalkyl, optionally substituted heterocycloalkyl, optionally substituted aryl, optionally substituted aralkyl, optionally substituted heteroaryl, optionally substituted heteroaralkyl, or R¹⁶ and R¹⁷ together form a carbocyclic or heterocyclic ring;

R²⁰ is hydroxyl, optionally substituted lower alkoxy, optionally substituted lower alkyl, optionally substituted lower alkenyl, optionally substituted lower alkynyl, optionally substituted cycloalkyl, optionally substituted heteroalkyl, optionally substituted heterocycloalkyl, optionally substituted aryl, optionally substituted aralkyl, optionally substituted heteroaryl, or optionally substituted heteroaralkyl; and

R²¹ is optionally substituted lower alkoxy, optionally substituted lower alkyl, optionally substituted lower alkenyl, optionally substituted lower alkynyl, optionally substituted cycloalkyl, optionally substituted heteroalkyl, optionally substituted heterocycloalkyl, optionally substituted aryl, optionally substituted aralkyl, optionally substituted heteroaryl, or optionally substituted heteroaralkyl.

In particular embodiments of compounds of Formula VII, with reference to Formula VII, R¹ is H; R² is H; R³ is H; R⁴ is H; R⁵ is H; R⁶ is H; R⁷ is H; each combination of two of R¹, R², R³, R⁴, R⁵, R⁶, and R⁷ are H; each combination of three of R¹, R², R³, R⁴, R⁵, R⁶, and R⁷ are H; each combination of 4 of R¹, R², R³, R⁴, R⁵, R⁶, and R⁷ are H; each combination of 5 of R¹, R², R³, R⁴, R⁵, R⁶, and R⁷ are H; each combination of six of R¹, R², R³, R⁴, R⁵, R⁶, and R⁷ are H (e.g., all except for R¹ and R⁶).

As used in connection with the present invention, unless indicated clearly to the contrary, the description of compounds and the use of such compounds, e.g., compounds of any of Formula I-VII, includes pharmaceutically acceptable salts and solvates of such compounds.

In connection with the compounds of Formulas I-VII, the following definitions apply.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH.

“Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1-15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups of 1-6, 1-4, or 1-2 carbon atoms. Preferably, cycloalkyl groups are monocyclic, bicyclic or tricyclic ring systems of 3-8, more preferably 3-6, ring members per ring, such as cyclopropyl, cyclopentyl, cyclohexyl, and the like, but can also include larger ring structures such as adamantyl. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to a straight chain alkynyl or branched alkynyl, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″ R″″, where R is lower alkylene, or substituted lower alkylene, R′, R′″, R′″ may independently be hydrogen, halogen, lower alkyl, substituted lower alkyl, acyl, aryl, substituted aryl, heteroaryl, or substituted heteroaryl as defined below.

“Alkyl alkynyl” refers to a groups —RCCR′ where R is lower alkylene or substituted lower alkylene, R′ is hydrogen, lower alkyl, substituted lower alkyl, acyl, aryl, substituted aryl, heteroaryl, or substituted heteroaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” or “thioalkoxy” denotes the group —SR, —S(O)ₙ₌₁₋₂—R, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” or substituted amine denotes the group NRR′, where R and R′ may independently by hydrogen, lower alkyl, substituted lower alkyl, aryl, substituted aryl, heteroaryl, or substituted heteroaryl as defined herein, acyl or sulfonyl.

“Amido” denotes the group —C(O)NRR′, where R and R′ may independently by hydrogen, lower alkyl, substituted lower alkyl, aryl, substituted aryl, heteroaryl, substituted heteroaryl as defined herein.

“Carboxyl” denotes the group —C(O)OR, where R is hydrogen, lower alkyl, substituted lower alkyl, aryl, substituted aryl, heteroaryl, and substituted heteroaryl as defined herein.

“Carbocyclic” refers to a saturated, unsaturated, or aromatic group having a single ring (e.g., phenyl) or multiple condensed rings where all ring atoms are carbon atoms, which can optionally be unsubstituted or substituted with, e.g., halogen, lower alkyl, lower alkoxy, alkylthio, acetylene, amino, amido, carboxyl, hydroxyl, aryl, aryloxy, heterocycle, heteroaryl, substituted heteroaryl, nitro, cyano, thiol, sulfamido and the like.

“Aryl”—alone or in combination means phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, heteroaryl, substituted heteroaryl, nitro, cyano, thiol, sulfamido and the like.

“Heteroaryl”—alone or in combination means a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound.

“Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydrofuranyl, dihydropyridinyl, piperidinyl, pyrrolidinyl, piperazinyl, dihydrobenzofuryl, dihydroindolyl, and the like. A substituted hetercyclyl contains a substituent nitrogen attached at an available carbon or nitrogen to produce a stable compound.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b]thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, heteroaryl, substituted heteroaryl, nitro, cyano, thiol, sulfamido and the like.

“Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, heteroaryl, substituted heteroaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, heteroaryl, substituted heteroaryl, nitro, cyano, thiol, sulfamido and the like.

“Heteroalkyl” refers to the group —R-Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, heteroaryl, substituted heteroaryl, nitro, cyano, thiol, sulfamido and the like.

“Heteroarylalkyl” refers to the group —R-HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, heteroaryl, substituted heteroaryl, nitro, cyano, thiol, sulfamido and the like.

“Cycloalkyl” refers to a cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, heteroaryl, substituted heteroaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

“Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, heteroaryl, substituted heteroaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, heteroaryl, substituted heteroaryl, nitro, cyano, thiol, sulfamido and the like.

“Alkyl cycloheteroalkyl” denotes the group —R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, heteroaryl, substituted heteroaryl, nitro, cyano, thiol, sulfamido and the like.

An additional aspect of this invention relates to pharmaceutical formulations or compositions, that include a therapeutically effective amount of a compound of Formula I, II, III, IV, V, VI, or VII, (or a compound within a sub-group of compounds within any of those generic formulas) and at least one pharmaceutically acceptable carrier or excipient. The composition can include a plurality of different pharmacalogically active compounds.

As used herein, the term “pharmaceutical composition” refers to a preparation that includes a therapeutically significant quantity of an active agent, that is prepared in a form adapted for administration to a subject. Thus, the preparation does not include any component or components in such quantity that a reasonably prudent medical practitioner would find the preparation unsuitable for administration to a normal subject. In many cases, such a pharmaceutical composition is a sterile preparation.

In a related aspect, the invention provides kits that include a pharmaceutical composition as described herein. In particular embodiments, the pharmaceutical composition is packaged, e.g., in a vial, bottle, flask, which may be further packaged, e.g., within a box, envelope, or bag; the pharmaceutical composition is approved by the U.S. Food and Drug Administration or similar regulatory agency for administration to a mammal, e.g., a human; the pharmaceutical composition is approved for administration to a mammal, e.g., a human for a kinase-mediated disease or condition; the kit includes written instructions or other indication that the composition is suitable or approved for administration to a mammal, e.g., a human, for a kinase-mediated disease or condition; the pharmaceutical composition is packaged in unit does or single dose form, e.g., single dose pills, capsules, or the like.

In another related aspect, compounds of any of Formulas I-VII can be used in the preparation of a medicament for the treatment of a kinase-mediated disease or condition or a disease or condition in which modulation of a kinase provides a therapeutic benefit.

In the present context, the term “therapeutically effective” indicates that the materials or amount of material is effective to prevent, alleviate, or ameliorate one or more symptoms of a disease or medical condition, and/or to prolong the survival of the subject being treated.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectibles.

“A pharmaceutically acceptable salt” is intended to mean a salt that retains the biological effectiveness of the free acids and bases of the specified compound and that is not biologically or otherwise unacceptable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sodium, chloride, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, .gamma.-hydroxybutyrates, glycollates, tartrates, methane-sulfonates, propanesulfonates, naphthalene-1-sulfonates, naphthalene-2-sulfonates, and mandelates.

The term “pharmaceutically acceptable metabolite” refers to a pharmacologically acceptable product, which may be an active product, produced through metabolism of a specified compound (or salt thereof) in the body of a subject or patient. Metabolites of a compound may be identified using routine techniques known in the art, and their activities determined using tests such as those described herein. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity, and can be further metabolized to provide an active metabolite.

Additional aspects and embodiments will be apparent from the following Detailed Description and from the claims.

### BRIEF DESCRIPTION OF THE DRAWINGS

FIG. 1 shows a schematic representation of AMP-PNP in the binding site of PIM-1, showing conserved interacting residues.

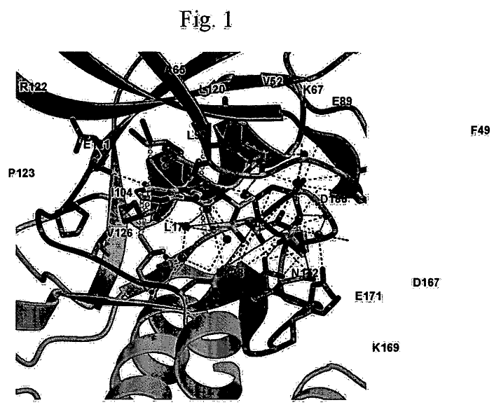

### DETAILED DESCRIPTION OF THE PREFERRED EMBODIMENTS

The Tables will first be briefly described.

Table 1 provides atomic coordinates for human PIM-1. In this table and in Table 4, the various columns have the following content, beginning with the left-most column:
ATOM: Refers to the relevant moiety for the table row.
Atom number: Refers to the arbitrary atom number designation within the coordinate table.
Atom Name: Identifier for the atom present at the particular coordinates.
Chain ID: Chain ID refers to one monomer of the protein in the crystal, e.g., chain “A”, or to other compound present in the crystal, e.g., HOH for water, and L for a ligand or binding compound. Multiple copies of the protein monomers will have different chain Ids.
Residue Number: The amino acid residue number in the chain.
X, Y, Z: Respectively are the X, Y, and Z coordinate values.
Occupancy: Describes the fraction of time the atom is observed in the crystal. For example, occupancy=1 means that the atom is present all the time; occupancy=o.5 indicates that the atom is present in the location 50% of the time.
B-factor: A measure of the thermal motion of the atom.
Element: Identifier for the element.

Table 2 provides an alignment of catalytic domains of several PIM kinases, including human PIM-1, PIM-2, and PIM-3 as well as PIM kinases from other species. Sequences from the following species are included in the alignment: Hs, Homo sapiens; Mm, Mus musculus; Dr, Danio rerio; Xl, Xenopus laevis; Cc, Cotumix cotumix; and Ce, Caenorhabditis elegans. Residues with >90% and >75% conservations are in red and yellow background, respectively. Phosphate binding sites are indicated by purple circles. Residues that are invariably involved in ligand binding are indicated by filled uparrows, whereas residues that can be involved in ligand binding are indicated by open uparrows. The backbone atoms of two residues (indicated by leftarrows) in the hinge region have been shown to make hydrogen bonds to ligands in many known kinase/ligand complex structures. Note that PIM family kinases all have Pro as the second residue, resulting in the loss of a hydrogen bond donor.

Table 3 provides alignments of a large set of kinases, providing identification of residues conserved between various members of the set.

Table 4 provides atomic coordinates for PIM-1 with AMP-PNP in the binding site.

Table 5 provides a list of kinases which have been correlated with diseases (or pathological condition).

I. Introduction

The present invention concerns the use of certain molecular scaffolds in the development of kinase modulators, e.g., kinase inhibitors. Such development can utilize kinase structures, for example, PIM kinase structures, structural information, and related compositions for identifying compounds that modulate kinase activity and for determining structures of other kinases. The following description utilizes PIM-1 for illustrative purposes. However, the invention is not limited to PIM-1; other protein kinases can also be utilized for modulation by the present compounds, and for developing additional modulators based on the present molecular scaffolds. The use of molecular scaffolds in connection with PIM kinases and Pyk2 is described in U.S. application Ser. No. 10/664,421 and corresponding PCT/US03/29415, and Formula I as described in U.S. application Ser. No. 10/789,818 and corresponding PCT/U.S. 2004/005904 respectively. Both of those applications are incorporated herein by reference in their entireties, including drawings.

Kinases, e.g., PIM-1, are involved in a variety of disease conditions, and a number have been utilized as therapeutic targets.

Exemplary Diseases Associated with Kinases

As indicated above, the present invention is exemplified in connection with PIM-1. As indicated in the Background above, PIM-1 functions as a weak oncogene. In transgenic mice with PIM-1 driven by Emu enhancer sequences, overexpression of PIM-1 by itself it does not lead to tumor formation, but does so in conjunction with overexpression of a second oncogenic gene. In 75% of tumors over-expressing PIM-1, the second gene found to be overexpressed was c-myc (van der Houven van Oordt C W, Schouten T G, van Krieken J H, van Dierendonck J H, van der Eb A J, Breuer M L. (1998) X-ray-induced lymphomagenesis in E mu-PIM-1 transgenic mice: an investigation of the co-operating molecular events. Carcinogenesis 19: 847-853). Other PIM kinases are also involved, as the functions of the various PIM kinases appears to be at least partially complementary.

Since PIM-1 is a protooncogene and it closely cooperates with other protooncogenes like c-myc in triggering intracellular signals leading to cell transformation, PIM-1 inhibitors have therapeutic applications in the treatment of various cancers, as wells as other disease states. Some examples are described below.

Prostate Cancer

A significant inter-relationship between PIM-1 and a disease state was reported in prostate cancer (Dhanasekaran et al. (2001) Delineation of prognostic biomarkers in prostate cancer. Nature 412: 822-826.) Using microarrays of complementary DNA, the gene expression profiles of approximately 10,000 genes from more than 50 normal and neoplastic prostate cancer specimens and three common prostate cancer cell lines were examined. Two of these genes, hepsin, a transmembrane serine protease, and PIM-1, a serine/threonine kinase are upregulated to several-fold. The PIM-1 kinase is strongly expressed in the cytoplasm of prostate cancer tissues while the normal tissues showed no or weak staining with anti-PIM-1 antibody (Id.) indicating PIM-1 is an appropriate target for drug development.

Leukemia

PIM-1 has been mapped to the 6p21 chromosomal region in humans. Nagarajan et al. (Nagarajan et al. (1986) Localization of the human pim oncogene (PIM) to a region of chromosome 6 involved in translocations in acute leukemias. Proc. Natl. Acad. Sci. USA 83: 2556-2560) reported increased expression of PIM-1 in K562 erythroleukemia cell lines which contain cytogenetically demonstrable rearrangement in the 6p21 region. A characteristic chromosome anomaly, a reciprocal translocation t(6;9)(p21;q33), has been described in myeloid leukemias that may be due to involvement of PIM-1. Amson et al. (1989) also observed overexpression in 30% of myeloid and lymphoid acute leukemia. These studies also indicate a role for PIM-1 protooncogene during development and in deregulation in various leukemias.

Kaposi Sarcoma

Analysis of gene expression profiles by microarrays in human hematopoietic cells after in vitro infection with human Herpes virus (HHV 8), also known as Kaposi Sarcoma associated virus (KSHV), resulted in differential expression of 400 genes out of about 10,000 analyzed. Of these four hundred genes, PIM-2 is upregulated more than 3.5 fold indicating PIM-2 as a potential target for therapeutic intervention. Thus, inhibitors selective to PIM-2 are of great therapeutic value in treating disease states mediated by HHV8 (Mikovits et al. (2001) Potential cellular signatures of viral infections in human hematopoietic cells. Dis. Markers 17: 173-178.)

Asthma and Allergy.

The increase in eosinophils at the site of antigen challenge has been used as evidence that eosinophils play a role in pathophysiology of asthma. Aberrant production of several different cytokines has been shown to result in eosinophilia. The cytokine IL-5 for example influences the development and maturation of eosinophils in a number of ways. Using microarray techniques, a role for PIM-1 in IL-5 signaling pathway in eosinophils was indicated. (Temple et al. (2001) Microarray analysis of eosinophils reveals a number of candidate survival and apoptosis genes. Am. J. Respir. Cell Mol. Biol. 25: 425-433.) Thus, inhibitors of PIM-1 can have therapeutic value in treatment of asthma and allergies.

Inflammation

PIM-1 and/or the compounds described herein can also be useful for treatment of inflammation, either chronic or acute. Chronic inflammation is regarded as prolonged inflammation (weeks or months), involving simultaneous active inflammation, tissue destruction, and attempts at healing. (R. S. Cotran, V. Kumar, and S. L. Robbins, Saunders Co., (1989) Robbins Pathological Basis of Disease, p. 75.) Although chronic inflammation can follow an acute inflammatory episode, it can also begin as a process that progresses over time, e.g., as a result of a chronic infection such as tuberculosis, syphilis, fungal infection which causes a delayed hypersensitivity reaction, prolonged exposure to endogenous or exogenous toxins, or autoimmune reactions (e.g., rheumatoid arthritis, systemic lupus erythematosus, multiple sclerosis, psoriasis). Chronic inflammatory disease thus include many common medical conditions such as autoimmune disorders such as those listed above, chronic infections, surgical adhesions, chronic inflammatory lung and airway diseases (e.g., asthma, pneumoconiosis, chronic obstructive pulmonary disease, nasal polyps, and pulmonary fibrosis). For skin and airway inflammatory disease, topical or inhaled forms of drug administration can be used respectively.

While kinase-related diseases and conditions are exemplified in connection with PIM-1, numerous other kinases have been correlated with particular disease and conditions, and are identified as target in a number of current treatments. Compounds derived from the present scaffolds can be developed to target such additional kinases for treating associated diseases and conditions.

II. Crystalline Kinases

In development of kinase modulators based on molecular scaffolds, crystalline kinases (e.g., human PIM-1) include native crystals, derivative crystals and co-crystals are useful. Native crystals generally comprise substantially pure polypeptides corresponding to the kinase in crystalline form. Crystal structures for a number of different kinases (or kinase domains) have been determined and are available for use in the present methods.

It is to be understood that the crystalline kinases are not limited to naturally occurring or native kinase. Indeed, the crystals of the invention include crystals of mutants of native kinases. Mutants of native kinases are obtained by replacing at least one amino acid residue in a native kinase with a different amino acid residue, or by adding or deleting amino acid residues within the native polypeptide or at the N- or C-terminus of the native polypeptide, and have substantially the same three-dimensional structure as the native kinase from which the mutant is derived.

By having substantially the same three-dimensional structure is meant having a set of atomic structure coordinates that have a root-mean-square deviation of less than or equal to about 2 Å when superimposed with the atomic structure coordinates of the native kinase from which the mutant is derived when at least about 50% to 100% of the Ca atoms of the native kinase domain are included in the superposition.

Amino acid substitutions, deletions and additions which do not significantly interfere with the three-dimensional structure of the kinase will depend, in part, on the region of the kinase where the substitution, addition or deletion occurs. In highly variable regions of the molecule, non-conservative substitutions as well as conservative substitutions may be tolerated without significantly disrupting the three-dimensional, structure of the molecule. In highly conserved regions, or regions containing significant secondary structure, conservative amino acid substitutions are preferred. Such conserved and variable regions can be identified by sequence alignment of a particular kinase (e.g., PIM-1) with other kinases). Such alignment of some kinases is provided in Table 3.

Conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

For kinases obtained in whole or in part by chemical synthesis, the selection of amino acids available for substitution or addition is not limited to the genetically encoded amino acids. Indeed, the mutants described herein may contain non-genetically encoded amino acids. Conservative amino acid substitutions for many of the commonly known non-genetically encoded amino acids are well known in the art. Conservative substitutions for other amino acids can be determined based on their physical properties as compared to the properties of the genetically encoded amino acids.

In some instances, it may be particularly advantageous or convenient to substitute, delete and/or add amino acid residues to a native kinase in order to provide convenient cloning sites in cDNA encoding the polypeptide, to aid in purification of the polypeptide, and for crystallization of the polypeptide. Such substitutions, deletions and/or additions which do not substantially alter the three dimensional structure of the native kinase domain will be apparent to those of ordinary skill in the art.

It should be noted that the mutants contemplated herein need not all exhibit kinase activity. Indeed, amino acid substitutions, additions or deletions that interfere with the kinase activity but which do not significantly alter the three-dimensional structure of the domain are specifically contemplated by the invention. Such crystalline polypeptides, or the atomic structure coordinates obtained therefrom, can be used to identify compounds that bind to the native domain. These compounds can affect the activity of the native domain.

The derivative crystals of the invention can comprise a crystalline kinase polypeptide in covalent association with one or more heavy metal atoms. The polypeptide may correspond to a native or a mutated kinase. Heavy metal atoms useful for providing derivative crystals include, by way of example and not limitation, gold, mercury, selenium, etc.

The co-crystals of the invention generally comprise a crystalline kinase domain polypeptide in association with one or more compounds. The association may be covalent or non-covalent. Such compounds include, but are not limited to, cofactors, substrates, substrate analogues, inhibitors, allosteric effectors, etc.

Exemplary mutations for PIM family kinases include the substitution or of the proline at the site corresponding to residue 123 in human PIM-1. One useful substitution is a proline to methionine substitution at residue 123 (P123M). Such substitution is useful, for example, to assist in using PIM family kinases to model other kinases that do not have proline at that site. Additional exemplary mutations include substitution or deletion of one or more of PIM-1 residues 124-128 or a residue from another PIM aligning with PIM-1 residues 124-128. For example, a PIM residue aligning with PIM-1 residue 128 can be deleted. Mutations at other sites can likewise be carried out, e.g., to make a mutated PIM family kinase more similar to another kinase for structure modeling and/or compound fitting purposes.

III. Three Dimensional Structure Determination Using X-Ray Crystallography

X-ray crystallography is a method of solving the three dimensional structures of molecules. The structure of a molecule is calculated from X-ray diffraction patterns using a crystal as a diffraction grating. Three dimensional structures of protein molecules arise from crystals grown from a concentrated aqueous solution of that protein. The process of X-ray crystallography can include the following steps:
(a) synthesizing and isolating (or otherwise obtaining) a polypeptide;
(b) growing a crystal from an aqueous solution comprising the polypeptide with or without a modulator; and
(c) collecting X-ray diffraction patterns from the crystals, determining unit cell dimensions and symmetry, determining electron density, fitting the amino acid sequence of the polypeptide to the electron density, and refining the structure.

Production of Polypeptides

The native and mutated kinase polypeptides described herein may be chemically synthesized in whole or part using techniques that are well-known in the art (see, e.g., Creighton (1983) Biopolymers 22(1): 49-58).

Alternatively, methods which are well known to those skilled in the art can be used to construct expression vectors containing the native or mutated kinase polypeptide coding sequence and appropriate transcriptional/translational control signals. These methods include in vitro recombinant DNA techniques, synthetic techniques and in vivo recombination/genetic recombination. See, for example, the techniques described in Maniatis, T (1989). Molecular cloning: A laboratory Manual. Cold Spring Harbor Laboratory, New York. Cold Spring Harbor Laboratory Press; and Ausubel, F. M. et al. (1994) Current Protocols in Molecular Biology. John Wiley & Sons, Secaucus, N.J.

A variety of host-expression vector systems may be utilized to express the kinase coding sequence. These include but are not limited to microorganisms such as bacteria transformed with recombinant bacteriophage DNA, plasmid DNA or cosmid DNA expression vectors containing the kinase domain coding sequence; yeast transformed with recombinant yeast expression vectors containing the kinase domain coding sequence; insect cell systems infected with recombinant virus expression vectors (e.g., baculovirus) containing the kinase domain coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the kinase domain coding sequence; or animal cell systems. The expression elements of these systems vary in their strength and specificities.

Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the ³⁵S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the kinase domain DNA, SV4O-, BPV- and EBV-based vectors may be used with an appropriate selectable marker.

Exemplary methods describing methods of DNA manipulation, vectors, various types of cells used, methods of incorporating the vectors into the cells, expression techniques, protein purification and isolation methods, and protein concentration methods are disclosed in detail in PCT publication WO 96/18738. This publication is incorporated herein by reference in its entirety, including any drawings. Those skilled in the art will appreciate that such descriptions are applicable to the present invention and can be easily adapted to it.

Crystal Growth

Crystals are grown from an aqueous solution containing the purified and concentrated polypeptide by a variety of techniques. These techniques include batch, liquid, bridge, dialysis, vapor diffusion, and hanging drop methods. McPherson (1982) John Wiley, New York; McPherson (1990) Eur. J. Biochem. 189: 1-23; Webber (1991) Adv. Protein Chem. 41: 1-36, incorporated by reference herein in their entireties, including all figures, tables, and drawings.

The native crystals of the invention are, in general, grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

For crystals of the invention, exemplary crystallization conditions are described in the Examples. Those of ordinary skill in the art will recognize that the exemplary crystallization conditions can be varied. Such variations may be used alone or in combination. In addition, other crystallizations may be found, e.g., by using crystallization screening plates to identify such other conditions.

Derivative crystals of the invention can be obtained by soaking native crystals in mother liquor containing salts of heavy metal atoms. It has been found that soaking a native crystal in a solution containing about 0.1 mM to about 5 mM thimerosal, 4-chloromeruribenzoic acid or KAu(CN)₂ for about 2 hr to about 72 hr provides derivative crystals suitable for use as isomorphous replacements in determining the X-ray crystal structure of PIM-1.

Co-crystals of the invention can be obtained by soaking a native crystal in mother liquor containing compound that binds the kinase, or can be obtained by co-crystallizing the kinase polypeptide in the presence of a binding compound.

Generally, co-crystallization of kinase and binding compound can be accomplished using conditions identified for crystallizing the corresponding kinase without binding compound. It is advantageous if a plurality of different crystallization conditions have been identified for the kinase, and these can be tested to determine which condition gives the best co-crystals. It may also be beneficial to optimize the conditions for co-crystallization. Exemplary co-crystallization conditions are provided in the Examples.

Determining Unit Cell Dimensions and the Three Dimensional Structure of a Polypeptide or Polypeptide Complex

Once the crystal is grown, it can be placed in a glass capillary tube or other mounting device and mounted onto a holding device connected to an X-ray generator and an X-ray detection device. Collection of X-ray diffraction patterns are well documented by those in the art. See, e.g., Ducruix and Geige, (1992), IRL Press, Oxford, England, and references cited therein. A beam of X-rays enters the crystal and then diffracts from the crystal. An X-ray detection device can be utilized to record the diffraction patterns emanating from the crystal. Although the X-ray detection device on older models of these instruments is a piece of film, modern instruments digitally record X-ray diffraction scattering. X-ray sources can be of various types, but advantageously, a high intensity source is used, e.g., a synchrotron beam source.

Methods for obtaining the three dimensional structure of the crystalline form of a peptide molecule or molecule complex are well known in the art. See, e.g., Ducruix and Geige, (1992), IRL Press, Oxford, England, and references cited therein. The following are steps in the process of determining the three dimensional structure of a molecule or complex from X-ray diffraction data.

After the X-ray diffraction patterns are collected from the crystal, the unit cell dimensions and orientation in the crystal can be determined. They can be determined from the spacing between the diffraction emissions as well as the patterns made from these emissions. The unit cell dimensions are characterized in three dimensions in units of Angstroms (one Å=10¹⁰ meters) and by angles at each vertices. The symmetry of the unit cell in the crystals is also characterized at this stage. The symmetry of the unit cell in the crystal simplifies the complexity of the collected data by identifying repeating patterns. Application of the symmetry and dimensions of the unit cell is described below.

Each diffraction pattern emission is characterized as a vector and the data collected at this stage of the method determines the amplitude of each vector. The phases of the vectors can be determined using multiple techniques. In one method, heavy atoms can be soaked into a crystal, a method called isomorphous replacement, and the phases of the vectors can be determined by using these heavy atoms as reference points in the X-ray analysis. (Otwinowski, (1991), Daresbury, United Kingdom, 80-86). The isomorphous replacement method usually utilizes more than one heavy atom derivative. In another method, the amplitudes and phases of vectors from a crystalline polypeptide with an already determined structure can be applied to the amplitudes of the vectors from a crystalline polypeptide of unknown structure and consequently determine the phases of these vectors. This second method is known as molecular replacement and the protein structure which is used as a reference must have a closely related structure to the protein of interest. (Naraza (1994) Proteins 11: 281-296). Thus, the vector information from a kinase of known structure, such as those reported herein, are useful for the molecular replacement analysis of another kinase with unknown structure.

Once the phases of the vectors describing the unit cell of a crystal are determined, the vector amplitudes and phases, unit cell dimensions, and unit cell symmetry can be used as terms in a Fourier transform function. The Fourier transform function calculates the electron density in the unit cell from these measurements. The electron density that describes one of the molecules or one of the molecule complexes in the unit cell can be referred to as an electron density map. The amino acid structures of the sequence or the molecular structures of compounds complexed with the crystalline polypeptide may then be fitted to the electron density using a variety of computer programs. This step of the process is sometimes referred to as model building and can be accomplished by using computer programs such as Turbo/FRODO or “O”. (Jones (1985) Methods in Enzymology 115: 157-171).

A theoretical electron density map can then be calculated from the amino acid structures fit to the experimentally determined electron density. The theoretical and experimental electron density maps can be compared to one another and the agreement between these two maps can be described by a parameter called an R-factor. A low value for an R-factor describes a high degree of overlapping electron density between a theoretical and experimental electron density map.

The R-factor is then minimized by using computer programs that refine the theoretical electron density map. A computer program such as X-PLOR can be used for model refinement by those skilled in the art. Bringer (1992) Nature 355: 472-475. Refinement may be achieved in an iterative process. A first step can entail altering the conformation of atoms defined in an electron density map. The conformations of the atoms can be altered by simulating a rise in temperature, which will increase the vibrational frequency of the bonds and modify positions of atoms in the structure. At a particular point in the atomic perturbation process, a force field, which typically defines interactions between atoms in terms of allowed bond angles and bond lengths, Van der Waals interactions, hydrogen bonds, ionic interactions, and hydrophobic interactions, can be applied to the system of atoms. Favorable interactions may be described in terms of free energy and the atoms can be moved over many iterations until a free energy minimum is achieved. The refinement process can be iterated until the R-factor reaches a minimum value.

The three dimensional structure of the molecule or molecule complex is described by atoms that fit the theoretical electron density characterized by a minimum R-value. A file can then be created for the three dimensional structure that defines each atom by coordinates in three dimensions. An example of such a structural coordinate file is shown in Table 1.

IV. Structures of an Exemplary Kinase Domain: Human PIM-1

As an example of kinase structure, high-resolution three-dimensional structures and atomic structure coordinates of crystalline PIM-1 and PIM-1 co-complexed with exemplary binding compounds as determined by X-ray crystallography are provided. The specific methods used to obtain the structure coordinates are provided in the examples. The atomic structure coordinates of crystalline PIM-1 are listed in Table 1, and atomic coordinates for PIM-1 co-crystallized with AMP-PMP are provided in Table 4. Co-crystal coordinates can be used in the same way, e.g., in the various aspects described herein, as coordinates for the protein by itself.

Those having skill in the art will recognize that atomic structure coordinates as determined by X-ray crystallography are not without error. Thus, it is to be understood that any set of structure coordinates obtained for crystals of PIM-1, whether native crystals, derivative crystals or co-crystals, that have a root mean square deviation (“r.m.s.d.”) of less than or equal to about 1.5 Å when superimposed, using backbone atoms (N, C_{α}, C and 0), on the structure coordinates listed in Table 1 (or Table 4) are considered to be identical with the structure coordinates listed in the Table 1 (or Table 4) when at least about 50% to 100% of the backbone atoms of PIM-1 are included in the superposition.

In addition to the PIM-1 structures provided herein, additional protein kinase structures are available and can be used, for example, publicly available structures deposited in the Protein Data Bank (PDB) (available for example, over the Internet). Higher quality structures are preferred (e.g., at least 2.5, 2.2, 2.0, 1.8, 1.7, 1.6, 1.5, 1.4, 1.3, or 1.2 Å resolution, as they provide more or more precise information for compound fitting and selection or design of derivatives.

V. Uses of the Crystals and Atomic Structure Coordinates

Kinase crystals, and particularly the atomic structure coordinates obtained therefrom, have a wide variety of uses. For example, the kinase crystals such as those described herein can be used as a starting point in any of the methods of use for kinases known in the art or later developed. Such methods of use include, for example, identifying molecules that bind to the native or mutated catalytic domain of kinases. The crystals and structure coordinates are particularly useful for identifying ligands that modulate kinase activity as an approach towards developing new therapeutic agents. In particular, the crystals and structural information are useful in methods for ligand development utilizing molecular scaffolds.

The structure coordinates described herein can be used as phasing models for determining the crystal structures of additional kinases, as well as the structures of co-crystals of such kinases with ligands such as inhibitors, agonists, antagonists, and other molecules. The structure coordinates, as well as models of the three-dimensional structures obtained therefrom, can also be used to aid the elucidation of solution-based structures of native or mutated kinases, such as those obtained via NMR.

VI. Electronic Representations of Kinase Structures

Structural information of kinases or portions of kinases (e.g., kinase active sites) can be represented in many different ways. Particularly useful are electronic representations, as such representations allow rapid and convenient data manipulations and structural modifications. Electronic representations can be embedded in many different storage or memory media, frequently computer readable media. Examples include without limitations, computer random access memory (RAM), floppy disk, magnetic hard drive, magnetic tape (analog or digital), compact disk (CD), optical disk, CD-ROM, memory card, digital video disk (DVD), and others. The storage medium can be separate or part of a computer system. Such a computer system may be a dedicated, special purpose, or embedded system, such as a computer system that forms part of an X-ray crystallography system, or may be a general purpose computer (which may have data connection with other equipment such as a sensor device in an X-ray crystallographic system. In many cases, the information provided by such electronic representations can also be represented physically or visually in two or three dimensions, e.g., on paper, as a visual display (e.g., on a computer monitor as a two dimensional or pseudo-three dimensional image) or as a three dimensional physical model. Such physical representations can also be used, alone or in connection with electronic representations. Exemplary useful representations include, but are not limited to, the following:

Atomic Coordinate Representation

One type of representation is a list or table of atomic coordinates representing positions of particular atoms in a molecular structure, portions of a structure, or complex (e.g., a co-crystal). Such a representation may also include additional information, for example, information about occupancy of particular coordinates.

Energy Surface or Surface of Interaction Representation

Another representation is an energy surface representation, e.g., of an active site or other binding site, representing an energy surface for electronic and steric interactions. Such a representation may also include other features. An example is the inclusion of representation of a particular amino acid residue(s) or group(s) on a particular amino acid residue(s), e.g., a residue or group that can participate in H-bonding or ionic interaction.

Structural Representation

Still another representation is a structural representation, i.e., a physical representation or an electronic representation of such a physical representation. Such a structural representation includes representations of relative positions of particular features of a molecule or complex, often with linkage between structural features. For example, a structure can be represented in which all atoms are linked; atoms other than hydrogen are linked; backbone atoms, with or without representation of side chain atoms that could participate in significant electronic interaction, are linked; among others. However, not all features need to be linked. For example, for structural representations of portions of a molecule or complex, structural features significant for that feature may be represented (e.g., atoms of amino acid residues that can have significant binding interaction with a ligand at a binding site. Those amino acid residues may not be linked with each other.

A structural representation can also be a schematic representation. For example, a schematic representation can represent secondary and/or tertiary structure in a schematic manner. Within such a schematic representation of a polypeptide, a particular amino acid residue(s) or group(s) on a residue(s) can be included, e.g., conserved residues in a binding site, and/or residue(s) or group(s) that may interact with binding compounds.

VII. Structure Determination for Kinases with Unknown Structure Using Structural Coordinates

Structural coordinates, such as those set forth in Table 1, can be used to determine the three dimensional structures of kinases with unknown structure. The methods described below can apply structural coordinates of a polypeptide with known structure to another data set, such as an amino acid sequence, X-ray crystallographic diffraction data, or nuclear magnetic resonance (NMR) data. Preferred embodiments of the invention relate to determining the three dimensional structures of other PIM kinases, other serine/threonine kinases, and related polypeptides.

Structures Using Amino Acid Homology

Homology modeling is a method of applying structural coordinates of a polypeptide of known structure to the amino acid sequence of a polypeptide of unknown structure. This method is accomplished using a computer representation of the three dimensional structure of a polypeptide or polypeptide complex, the computer representation of amino acid sequences of the polypeptides with known and unknown structures, and standard computer representations of the structures of amino acids. Homology modeling generally involves (a) aligning the amino acid sequences of the polypeptides with and without known structure; (b) transferring the coordinates of the conserved amino acids in the known structure to the corresponding amino acids of the polypeptide of unknown structure; refining the subsequent three dimensional structure; and (d) constructing structures of the rest of the polypeptide. One skilled in the art recognizes that conserved amino acids between two proteins can be determined from the sequence alignment step in step (a).

The above method is well known to those skilled in the art. (Greer (1985) Science 228: 1055; Blundell et al. A (1988) Eur. J. Biochemi. 172: 513. An exemplary computer program that can be utilized for homology modeling by those skilled in the art is the Homology module in the Insight II modeling package distributed by Accelerys Inc.

Alignment of the amino acid sequence is accomplished by first placing the computer representation of the amino acid sequence of a polypeptide with known structure above the amino acid sequence of the polypeptide of unknown structure. Amino acids in the sequences are then compared and groups of amino acids that are homologous (e.g., amino acid side chains that are similar in chemical nature—aliphatic, aromatic, polar, or charged) are grouped together. This method will detect conserved regions of the polypeptides and account for amino acid insertions or deletions.

Once the amino acid sequences of the polypeptides with known and unknown structures are aligned, the structures of the conserved amino acids in the computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions are to be assigned manually by either using standard peptide geometries or molecular simulation techniques, such as molecular dynamics. The final step in the process is accomplished by refining the entire structure using molecular dynamics and/or energy minimization. The homology modeling method is well known to those skilled in the art and has been practiced using different protein molecules. For example, the three dimensional structure of the polypeptide corresponding to the catalytic domain of a serine/threonine protein kinase, myosin light chain protein kinase, was homology modeled from the cAMP-dependent protein kinase catalytic subunit. (Knighton et al. (1992) Science 258: 130-135.)

Structures Using Molecular Replacement

Molecular replacement is a method of applying the X-ray diffraction data of a polypeptide of known structure to the X-ray diffraction data of a polypeptide of unknown sequence. This method can be utilized to define the phases describing the X-ray diffraction data of a polypeptide of unknown structure when only the amplitudes are known. X-PLOR is a commonly utilized computer software package used for molecular replacement. Brunger (1992) Nature 355: 472-475. AMORE is another program used for molecular replacement. Navaza (1994) Acta Crystallogr. A50: 157-163. Preferably, the resulting structure does not exhibit a root-mean-square deviation of more than 3 Å.

A goal of molecular replacement is to align the positions of atoms in the unit cell by matching electron diffraction data from two crystals. A program such as X-PLOR can involve four steps. A first step can be to determine the number of molecules in the unit cell and define the angles between them. A second step can involve rotating the diffraction data to define the orientation of the molecules in the unit cell. A third step can be to translate the electron density in three dimensions to correctly position the molecules in the unit cell. Once the amplitudes and phases of the X-ray diffraction data is determined, an R-factor can be calculated by comparing electron diffraction maps calculated experimentally from the reference data set and calculated from the new data set. An R-factor between 30-50% indicates that the orientations of the atoms in the unit cell are reasonably determined by this method. A fourth step in the process can be to decrease the R-factor to roughly 20% by refining the new electron density map using iterative refinement techniques described herein and known to those or ordinary skill in the art.

Structures Using NMR Data

Structural coordinates of a polypeptide or polypeptide complex derived from X-ray crystallographic techniques can be applied towards the elucidation of three dimensional structures of polypeptides from nuclear magnetic resonance (NMR) data. This method is used by those skilled in the art. (Wuthrich, (1986), John Wiley and Sons, New York: 176-199; Pflugrath et al. (1986) J. Mol. Biol. 189: 383-386; Kline et al. (1986) J. Mol. Biol. 189: 377-382). While the secondary structure of a polypeptide is often readily determined by utilizing two-dimensional NMR data, the spatial connections between individual pieces of secondary structure are not as readily determinable. The coordinates defining a three-dimensional structure of a polypeptide derived from X-ray crystallographic techniques can guide the NMR spectroscopist to an understanding of these spatial interactions between secondary structural elements in a polypeptide of related structure.

The knowledge of spatial interactions between secondary structural elements can greatly simplify Nuclear Overhauser Effect (NOE) data from two-dimensional NMR experiments. Additionally, applying the crystallographic coordinates after the determination of secondary structure by NMR techniques only simplifies the assignment of NOEs relating to particular amino acids in the polypeptide sequence and does not greatly bias the NMR analysis of polypeptide structure. Conversely, using the crystallographic coordinates to simplify NOE data while determining secondary structure of the polypeptide would bias the NMR analysis of protein structure.

VIII. Structure-Based Design of Modulators of Kinase Function Utilizing Structural Coordinates

Structure-based modulator design and identification methods are powerful techniques that can involve searches of computer databases containing a wide variety of potential modulators and chemical functional groups. The computerized design and identification of modulators is useful as the computer databases contain more compounds than the chemical libraries, often by an order of magnitude. For reviews of structure-based drug design and identification (see Kuntz et al. (1994), Acc. Chem. Res. 27: 117; Guida (1994) Current Opinion in Struc. Biol. 4: 777; Colman (1994) Current Opinion in Struc. Biol. 4: 868).

The three dimensional structure of a polypeptide defined by structural coordinates can be utilized by these design methods, for example, the structural coordinates of Table 1. In addition, the three dimensional structures of kinases determined by the homology, molecular replacement, and NMR techniques described herein can also be applied to modulator design and identification methods.

For identifying modulators, structural information for a native kinase, in particular, structural information for the active site of the kinase, can be used. However, it may be advantageous to utilize structural information from one or more co-crystals of the kinase with one or more binding compounds. It can also be advantageous if the binding compound has a structural core in common with test compounds.

Design by Searching Molecular Data Bases

One method of rational design searches for modulators by docking the computer representations of compounds from a database of molecules. Publicly available databases include, for example:
a) ACD from Molecular Designs Limited
b) NCI from National Cancer Institute
c) CCDC from Cambridge Crystallographic Data Center
d) CAST from Chemical Abstract Service
e) Derwent from Derwent Information Limited
f) Maybridge from Maybridge Chemical Company LTD
g) Aldrich from Aldrich Chemical Company
h) Directory of Natural Products from Chapman & Hall

One such data base (ACD distributed by Molecular Designs Limited Information Systems) contains compounds that are synthetically derived or are natural products. Methods available to those skilled in the art can convert a data set represented in two dimensions to one represented in three dimensions. These methods are enabled by such computer programs as CONCORD from Tripos Associates or DE-Converter from Molecular Simulations Limited.

Multiple methods of structure-based modulator design are known to those in the art. (Kuntz et al., (1982), J. Mol. Biol. 162: 269; Kuntz et al., (1994), Acc. Chem. Res. 27: 117; Meng et al., (1992), J Compt. Chem. 13: 505; Bohm, (1994), J. Comp. Aided Molec. Design 8: 623).

A computer program widely utilized by those skilled in the art of rational modulator design is DOCK from the University of California in San Francisco. The general methods utilized by this computer program and programs like it are described in three applications below. More detailed information regarding some of these techniques can be found in the Accelerys User Guide, 1995. A typical computer program used for this purpose can comprise the following steps:
(a) remove the existing compound from the protein;
(b) dock the structure of another compound into the active-site using the computer program (such as DOCK) or by interactively moving the compound into the active-site;
(c) characterize the space between the compound and the active-site atoms;
(d) search libraries for molecular fragments which (i) can fit into the empty space between the compound and the active-site, and (ii) can be linked to the compound; and
(e) link the fragments found above to the compound and evaluate the new modified compound.

Part (c) refers to characterizing the geometry and the complementary interactions formed between the atoms of the active site and the compounds. A favorable geometric fit is attained when a significant surface area is shared between the compound and active-site atoms without forming unfavorable steric interactions. One skilled in the art would note that the method can be performed by skipping parts (d) and (e) and screening a database of many compounds.

Structure-based design and identification of modulators of kinase function can be used in conjunction with assay screening. As large computer databases of compounds (around 10,000 compounds) can be searched in a matter of hours, the computer-based method can narrow the compounds tested as potential modulators of kinase function in biochemical or cellular assays.

The above descriptions of structure-based modulator design are not all encompassing and other methods are reported in the literature:
(1) CAVEAT: Bartlett et al., (1989), in Chemical and Biological Problems in Molecular Recognition, Roberts, S. M.; Ley, S. V.; Campbell, M. M. eds.; Royal Society of Chemistry: Cambridge, pp 182-196.
(2) FLOG: Miller et al., (1994), J. Comp. Aided Molec. Design 8: 153.
(3) PRO Modulator: Clark et al., (1995), J. Comp. Aided Molec. Design 9: 13.
(4) MCSS: Miranker and Karplus, (1991), Proteins: Structure, Function, and Genetics 11: 29.
(5) AUTODOCK: Goodsell and Olson, (1990), Proteins. Structure, Function, and Genetics 80.195.
(6) GRID: Goodford, (1985), J. Med. Chem. 28: 849.

Design by Modifying Compounds in Complex with a Kinase

Another way of identifying compounds as potential modulators is to modify an existing modulator in the polypeptide active site. For example, the computer representation of modulators can be modified within the computer representation of a PIM-1 or other PIM kinase active site. Detailed instructions for this technique can be found in the Accelerys User Manual, 1995 in LUDI. The computer representation of the modulator is typically modified by the deletion of a chemical group or groups or by the addition of a chemical group or groups.

Upon each modification to the compound, the atoms of the modified compound and active site can be shifted in conformation and the distance between the modulator and the active-site atoms may be scored along with any complementary interactions formed between the two molecules. Scoring can be complete when a favorable geometric fit and favorable complementary interactions are attained. Compounds that have favorable scores are potential modulators.

Design by Modifying the Structure of Compounds that Bind a Kinase

A third method of structure-based modulator design is to screen compounds designed by a modulator building or modulator searching computer program. Examples of these types of programs can be found in the Molecular Simulations Package, Catalyst. Descriptions for using this program are documented in the Molecular Simulations User Guide (1995). Other computer programs used in this application are ISIS/HOST, ISIS/BASE, ISIS/DRAW) from Molecular Designs Limited and UNITY from Tripos Associates.

These programs can be operated on the structure of a compound that has been removed from the active site of the three dimensional structure of a compound-kinase complex. Operating the program on such a compound is preferable since it is in a biologically active conformation.

A modulator construction computer program is a computer program that may be used to replace computer representations of chemical groups in a compound complexed with a kinase or other biomolecule with groups from a computer database. A modulator searching computer program is a computer program that may be used to search computer representations of compounds from a computer data base that have similar three dimensional structures and similar chemical groups as compound bound to a particular biomolecule.

A typical program can operate by using the following general steps:
(a) map the compounds by chemical features such as by hydrogen bond donors or acceptors, hydrophobic/lipophilic sites, positively ionizable sites, or negatively ionizable sites;
(b) add geometric constraints to the mapped features; and
(c) search databases with the model generated in (b).

Those skilled in the art also recognize that not all of the possible chemical features of the compound need be present in the model of (b). One can use any subset of the model to generate different models for data base searches.

Modulator Design Using Molecular Scaffolds

The present invention can also advantageously utilize methods for designing compounds, designated as molecular scaffolds, that can act broadly across families of molecules and for using the molecular scaffold to design ligands that target individual or multiple members of those families. In preferred embodiments, the molecules can be proteins and a set of chemical compounds can be assembled that have properties such that they are 1) chemically designed to act on certain protein families and/or 2) behave more like molecular scaffolds, meaning that they have chemical substructures that make them specific for binding to one or more proteins in a family of interest. Alternatively, molecular scaffolds can be designed that are preferentially active on an individual target molecule.

Useful chemical properties of molecular scaffolds can include one or more of the following characteristics, but are not limited thereto: an average molecular weight below about 350 daltons, or between from about 150 to about 350 daltons, or from about 150 to about 300 daltons; having a clogP below 3; a number of rotatable bonds of less than 4; a number of hydrogen bond donors and acceptors below 5 or below 4; a polar surface area of less than 50 Å²; binding at protein binding sites in an orientation so that chemical substituents from a combinatorial library that are attached to the scaffold can be projected into pockets in the protein binding site; and possessing chemically tractable structures at its substituent attachment points that can be modified, thereby enabling rapid library construction.

By “clog P” is meant the calculated log P of a compound, “P” referring to the partition coefficient between octanol and water.

The term “Molecular Polar Surface Area (PSA)” refers to the sum of surface contributions of polar atoms (usually oxygens, nitrogens and attached hydrogens) in a molecule. The polar surface area has been shown to correlate well with drug transport properties, such as intestinal absorption, or blood-brain barrier penetration.

Additional useful chemical properties of distinct compounds for inclusion in a combinatorial library include the ability to attach chemical moieties to the compound that will not interfere with binding of the compound to at least one protein of interest, and that will impart desirable properties to the library members, for example, causing the library members to be actively transported to cells and/or organs of interest, or the ability to attach to a device such as a chromatography column (e.g., a streptavidin column through a molecule such as biotin) for uses such as tissue and proteomics profiling purposes.

A person of ordinary skill in the art will realize other properties that can be desirable for the scaffold or library members to have depending on the particular requirements of the use, and that compounds with these properties can also be sought and identified in like manner. Methods of selecting compounds for assay are known to those of ordinary skill in the art, for example, methods and compounds described in U.S. Pat. Nos. 6,288,234, 6,090,912, 5,840,485, each of which is hereby incorporated by reference in its entirety, including all charts and drawings.

In various embodiments, the present invention provides methods of designing ligands that bind to a plurality of members of a molecular family, where the ligands contain a common molecular scaffold. Thus, a compound set can be assayed for binding to a plurality of members of a molecular family, e.g., a protein family. One or more compounds that bind to a plurality of family members can be identified as molecular scaffolds. When the orientation of the scaffold at the binding site of the target molecules has been determined and chemically tractable structures have been identified, a set of ligands can be synthesized starting with one or a few molecular scaffolds to arrive at a plurality of ligands, wherein each ligand binds to a separate target molecule of the molecular family with altered or changed binding affinity or binding specificity relative to the scaffold. Thus, a plurality of drug lead molecules can be designed to preferentially target individual members of a molecular family based on the same molecular scaffold, and act on them in a specific manner.

Binding Assays

The methods of the present invention can involve assays that are able to detect the binding of compounds to a target molecule at a signal of at least about three times the standard deviation of the background signal, or at least about four times the standard deviation of the background signal. The assays of the present invention can also include assaying compounds for low affinity binding to the target molecule. A large variety of assays indicative of binding are known for different target types and can be used for this invention. Compounds that act broadly across protein families are not likely to have a high affinity against individual targets, due to the broad nature of their binding. Thus, assays described herein allow for the identification of compounds that bind with low affinity, very low affinity, and extremely low affinity. Therefore, potency (or binding affinity) is not the primary, nor even the most important, indicia of identification of a potentially useful binding compound. Rather, even those compounds that bind with low affinity, very low affinity, or extremely low affinity can be considered as molecular scaffolds that can continue to the next phase of the ligand design process.

By binding with “low affinity” is meant binding to the target molecule with a dissociation constant (k_{d}) of greater than 1 μM under standard conditions. By binding with “very low affinity” is meant binding with a k_{d} of above about 100 μM under standard conditions. By binding with “extremely low affinity” is meant binding at a k_{d} of above about 1 mM under standard conditions. By “moderate affinity” is meant binding with a k_{d} of from about 200 nM to about 1 μM under standard conditions. By “moderately high affinity” is meant binding at a k_{d} of from about 1 nM to about 200 nM. By binding at “high affinity” is meant binding at a k_{d} of below about 1 nM under standard conditions. For example, low affinity binding can occur because of a poorer fit into the binding site of the target molecule or because of a smaller number of non-covalent bonds, or weaker covalent bonds present to cause binding of the scaffold or ligand to the binding site of the target molecule relative to instances where higher affinity binding occurs. The standard conditions for binding are at pH 7.2 at 37° C. for one hour. For example, 100 μl/well can be used in HEPES 50 mM buffer at pH 7.2, NaCl 15 mM, ATP 2 μM, and bovine serum albumin 1 ug/well, 37° C. for one hour.

Binding compounds can also be characterized by their effect on the activity of the target molecule. Thus, a “low activity” compound has an inhibitory concentration (IC₅₀) or excitation concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 μM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667$$

To design or discover scaffolds that act broadly across protein families, proteins of interest can be assayed against a compound collection or set. The assays can preferably be enzymatic or binding assays. In some embodiments it may be desirable to enhance the solubility of the compounds being screened and then analyze all compounds that show activity in the assay, including those that bind with low affinity or produce a signal with greater than about three times the standard deviation of the background signal. The assays can be any suitable assay such as, for example, binding assays that measure the binding affinity between two binding partners. Various types of screening assays that can be useful in the practice of the present invention are known in the art, such as those described in U.S. Pat. Nos. 5,763,198, 5,747,276, 5,877,007, 6,243,980, 6,294,330, and 6,294,330, each of which is hereby incorporated by reference in its entirety, including all charts and drawings.

In various embodiments of the assays at least one compound, at least about 5%, at least about 10%, at least about 15%, at least about 20%, or at least about 25% of the compounds can bind with low affinity. In general, up to about 20% of the compounds can show activity in the screening assay and these compounds can then be analyzed directly with high-throughput co-crystallography, computational analysis to group the compounds into classes with common structural properties (e.g., structural core and/or shape and polarity characteristics), and the identification of common chemical structures between compounds that show activity.

The person of ordinary skill in the art will realize that decisions can be based on criteria that are appropriate for the needs of the particular situation, and that the decisions can be made by computer software programs. Classes can be created containing almost any number of scaffolds, and the criteria selected can be based on increasingly exacting criteria until an arbitrary number of scaffolds is arrived at for each class that is deemed to be advantageous.

Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., (2000) BIAcore® analysis to test phosphopeptide-SH2 domain interactions, Methods in Molecular Biology. 121: 313-21; Liparoto et al., (1999) Biosensor analysis of the interleukin-2 receptor complex, Journal of Molecular Recognition. 12: 316-21; Lipschultz et al., (2000) Experimental design for analysis of complex kinetics using surface plasmon resonance, Methods. 20(3): 310-8; Malmqvist., (1999) BIACORE: an affinity biosensor system for characterization of biomolecular interactions, Biochemical Society Transactions 27: 335-40; Alfthan, (1998) Surface plasmon resonance biosensors as a tool in antibody engineering, Biosensors & Bioelectronics. 13: 653-63; Fivash et al., (1998) BIAcore for macromolecular interaction, Current Opinion in Biotechnology. 9: 97-101; Price et al.; (1998) Summary report on the ISOBM TD-4 Workshop: analysis of 56 monoclonal antibodies against the MUC1 mucin. Tumour Biology 19 Suppl 1: 1-20; Malmqvist et al, (1997) Biomolecular interaction analysis: affinity biosensor technologies for functional analysis of proteins, Current Opinion in Chemical Biology. 1: 378-83; O'Shannessy et al., (1996) Interpretation of deviations from pseudo-first-order kinetic behavior in the characterization of ligand binding by biosensor technology, Analytical Biochemistry. 236: 275-83; Malmborg et al., (1995) BIAcore as a tool in antibody engineering, Journal of Immunological Methods. 183: 7-13; Van Regenmortel, (1994) Use of biosensors to characterize recombinant proteins, Developments in Biological Standardization. 83: 143-51; and O'Shannessy, (1994) Determination of kinetic rate and equilibrium binding constants for macromolecular interactions: a critique of the surface plasmon resonance literature, Current Opinions in Biotechnology. 5: 65-71.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. Similarly, when ligands to a sphingolipid target are sought, known ligands of the target can be present in control/calibration assay wells.

Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of calorimetric assays for the detection of peroxides, as disclosed in Example 1(b) and Gordon, A. J. and Ford, R. A., (1972) The Chemist's Companion: A Handbook Of Practical Data, Techniques, And References, John Wiley and Sons, N.Y., Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., (1987) Spectrophotometry and Spectrofluorometry: A Practical Approach, pp. 91-114, IRL Press Ltd.; and Bell, (1981) Spectroscopy In Biochemistr, Vol. I, pp. 155-194, CRC Press.

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., (1997), Application of Fluorescence Polarization Assays in High-Throughput Screening, Genetic Engineering News, 17: 27.

FP is particularly desirable since its readout is independent of the emission intensity (Checovich, W. J., et al., (1995) Nature 375: 254-256; Dandliker, W. B., et al., (1981) Methods in Enzymology 74: 3-28) and is thus insensitive to the presence of colored compounds that quench fluorescence emission. FP and FRET (see below) are well-suited for identifying compounds that block interactions between sphingolipid receptors and their ligands. See, for example, Parker et al., (2000) Development of high throughput screening assays using fluorescence polarization: nuclear receptor-ligand-binding and kinase/phosphatase assays, J Biomol Screen 5: 77-88.

Fluorophores derived from sphingolipids that may be used in FP assays are commercially available. For example, Molecular Probes (Eugene, Oreg.) currently sells sphingomyelin and one ceramide fluorophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY® FL C12-sphingomyelin); and N-(4,4-difluoro-5,7-dimethyl-4-bora-3a,4a-diaza-s-indacene-3-pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan A G, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., (1996) Curr. Biol. 6: 178-182; Mitra et al., (1996) Gene 173: 13-17; and Selvin et al., (1995) Meth. Enzymol. 246: 300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., (1997) J. Lipid Res. 38: 2365-2373; Kahl et al., (1996) Anal. Biochem. 243: 282-283; Undenfriend et al., (1987) Anal. Biochem. 161: 494-500). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., (1998) Anal. Biochem. 257: 112-119).

Assay Compounds and Molecular Scaffolds

Preferred characteristics of a scaffold include being of low molecular weight (e.g., less than 350 Da, or from about 100 to about 350 daltons, or from about 150 to about 300 daltons). Preferably clog P of a scaffold is from −1 to 8, more preferably less than 6, 5, or 4, most preferably less than 3. In particular embodiments the clogP is in a range −1 to an upper limit of 2, 3, 4, 5, 6, or 8; or is in a range of 0 to an upper limit of 2, 3, 4, 5, 6, or 8. Preferably the number of rotatable bonds is less than 5, more preferably less than 4. Preferably the number of hydrogen bond donors and acceptors is below 6, more preferably below 5. An additional criterion that can be useful is a polar surface area of less than 5. Guidance that can be useful in identifying criteria for a particular application can be found in Lipinski et al., (1997) Advanced Drug Delivery Reviews 23 3-25, which is hereby incorporated by reference in its entirety.

A scaffold may preferably bind to a given protein binding site in a configuration that causes substituent moieties of the scaffold to be situated in pockets of the protein binding site. Also, possessing chemically tractable groups that can be chemically modified, particularly through synthetic reactions, to easily create a combinatorial library can be a preferred characteristic of the scaffold. Also preferred can be having positions on the scaffold to which other moieties can be attached, which do not interfere with binding of the scaffold to the protein(s) of interest but do cause the scaffold to achieve a desirable property, for example, active transport of the scaffold to cells and/or organs, enabling the scaffold to be attached to a chromatographic column to facilitate analysis, or another desirable property. A molecular scaffold can bind to a target molecule with any affinity, such as binding with an affinity measurable as about three times the standard deviation of the background signal, or at high affinity, moderate affinity, low affinity, very low affinity, or extremely low affinity.

Thus, the above criteria can be utilized to select many compounds for testing that have the desired attributes. Many compounds having the criteria described are available in the commercial market, and may be selected for assaying depending on the specific needs to which the methods are to be applied.

A “compound library” or “library” is a collection of different compounds having different chemical structures. A compound library is screenable, that is, the compound library members therein may be subject to screening assays. In preferred embodiments, the library members can have a molecular weight of from about 100 to about 350 daltons, or from about 150 to about 350 daltons. Examples of libraries are provided above.

Libraries of the present invention can contain at least one compound than binds to the target molecule at low affinity. Libraries of candidate compounds can be assayed by many different assays, such as those described above, e.g., a fluorescence polarization assay. Libraries may consist of chemically synthesized peptides, peptidomimetics, or arrays of combinatorial chemicals that are large or small, focused or nonfocused. By “focused” it is meant that the collection of compounds is prepared using the structure of previously characterized compounds and/or pharmacophores.

Compound libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized. Types of molecules in compound libraries include but are not limited to organic compounds, polypeptides and nucleic acids as those terms are used herein, and derivatives, conjugates and mixtures thereof.

Compound libraries of the invention may be purchased on the commercial market or prepared or obtained by any means including, but not limited to, combinatorial chemistry techniques, fermentation methods, plant and cellular extraction procedures and the like (see, e.g., Cwirla et al., (1990) Biochemistry, 87, 6378-6382; Houghten et al., (1991) Nature, 354, 84-86; Lam et al., (1991) Nature, 354, 82-84; Brenner et al., (1992) Proc. Natl. Acad. Sci. USA, 89, 5381-5383; R. A. Houghten, (1993) Trends Genet., 9, 235-239; E. R. Felder, (1994) Chimia, 48, 512-541; Gallop et al., (1994) J. Med. Chem., 37, 1233-1251; Gordon et al., (1994) J. Med Chem., 37, 1385-1401; Carell et al., (1995) Chem. Biol., 3, 171-183; Madden et al., Perspectives in Drug Discovery and Design 2, 269-282; Lebl et al., (1995) Biopolymers, 37 177-198); small molecules assembled around a shared molecular structure; collections of chemicals that have been assembled by various commercial and noncommercial groups, natural products; extracts of marine organisms, fingi, bacteria, and plants.

Preferred libraries can be prepared in a homogenous reaction mixture, and separation of unreacted reagents from members of the library is not required prior to screening. Although many combinatorial chemistry approaches are based on solid state chemistry, liquid phase combinatorial chemistry is capable of generating libraries (Sun C M., (1999) Recent advances in liquid-phase combinatorial chemistry, Combinatorial Chemistry & High Throughput Screening. 2: 299-318).

Libraries of a variety of types of molecules are prepared in order to obtain members therefrom having one or more preselected attributes that can be prepared by a variety of techniques, including but not limited to parallel array synthesis (Houghton, (2000) Annu Rev Pharmacol Toxicol 40: 273-82, Parallel array and mixture-based synthetic combinatorial chemistry; solution-phase combinatorial chemistry (Merritt, (1998) Comb Chem High Throughput Screen 1(2): 57-72, Solution phase combinatorial chemistry, Coe et al., (1998-99) Mol Divers; 4(1): 31-8, Solution-phase combinatorial chemistry, Sun, (1999) Comb Chem High Throughput Screen 2(6): 299-318, Recent advances in liquid-phase combinatorial chemistry); synthesis on soluble polymer (Gravert et al., (1997) Curr Opin Chem Biol 1(1): 107-13, Synthesis on soluble polymers: new reactions and the construction of small molecules); and the like. See, e.g., Dolle et al., (1999) J Comb Chem 1(4): 235-82, Comprehensive survey of combinatorial library synthesis: 1998. Freidinger R M., (1999) Nonpeptidic ligands for peptide and protein receptors, Current Opinion in Chemical Biology; and Kundu et al., Prog Drug Res; 53: 89-156, Combinatorial chemistry: polymer supported synthesis of peptide and non-peptide libraries). Compounds may be clinically tagged for ease of identification (Chabala, (1995) Curr Opin Biotechnol 6(6): 633-9, Solid-phase combinatorial chemistry and novel tagging methods for identifying leads).

The combinatorial synthesis of carbohydrates and libraries containing oligosaccharides have been described (Schweizer et al., (1999) Curr Opin Chem Biol 3(3): 291-8, Combinatorial synthesis of carbohydrates). The synthesis of natural-product based compound libraries has been described (Wessjohann, (2000) Curr Opin Chem Biol 4(3): 303-9, Synthesis of natural-product based compound libraries).

Libraries of nucleic acids are prepared by various techniques, including by way of non-limiting example the ones described herein, for the isolation of aptamers. Libraries that include oligonucleotides and polyaminooligonucleotides (Markiewicz et al., (2000) Synthetic oligonucleotide combinatorial libraries and their applications, Farmaco. 55: 174-7) displayed on streptavidin magnetic beads are known. Nucleic acid libraries are known that can be coupled to parallel sampling and be deconvoluted without complex procedures such as automated mass spectrometry (Enjalbal C. Martinez J. Aubagnac J L, (2000) Mass spectrometry in combinatorial chemistry, Mass Spectrometry Reviews. 19: 139-61) and parallel tagging. (Perrin D M., Nucleic acids for recognition and catalysis: landmarks, limitations, and looking to the future, Combinatorial Chemistry & High Throughput Screening 3: 243-69).

Peptidomimetics are identified using combinatorial chemistry and solid phase synthesis (Kim H O. Kahn M., (2000) A merger of rational drug design and combinatorial chemistry: development and application of peptide secondary structure mimetics, Combinatorial Chemistry & High Throughput Screening 3: 167-83; al-Obeidi, (1998) Mol Biotechnol 9(3): 205-23, Peptide and peptidomimetic libraries. Molecular diversity and drug design). The synthesis may be entirely random or based in part on a known polypeptide.

Polypeptide libraries can be prepared according to various techniques. In brief, phage display techniques can be used to produce polypeptide ligands (Gram H., (1999) Phage display in proteolysis and signal transduction, Combinatorial Chemistry & High Throughput Screening. 2: 19-28) that may be used as the basis for synthesis of peptidomimetics. Polypeptides, constrained peptides, proteins, protein domains, antibodies, single chain antibody fragments, antibody fragments, and antibody combining regions are displayed on filamentous phage for selection.

Large libraries of individual variants of human single chain Fv antibodies have been produced. See, e.g., Siegel R W. Allen B. Pavlik P. Marks J D. Bradbury A., (2000) Mass spectral analysis of a protein complex using single-chain antibodies selected on a peptide target: applications to functional genomics, Journal of Molecular Biology 302: 285-93; Poul M A. Becerril B. Nielsen U B. Morisson P. Marks J D., (2000) Selection of tumor-specific internalizing human antibodies from phage libraries. Source Journal of Molecular Biology. 301: 1149-61; Amersdorfer P. Marks J D., (2001) Phage libraries for generation of anti-botulinum scFv antibodies, Methods in Molecular Biology. 145: 219-40; Hughes-Jones N C. Bye J M. Gorick B D. Marks J D. Ouwehand W H., (1999) Synthesis of Rh Fv phage-antibodies using VH and VL germline genes, British Journal of Haematology. 105: 811-6; McCall A M. Amoroso A R. Sautes C. Marks J D. Weiner L M., (1998) Characterization of anti-mouse Fc gamma RII single-chain Fv fragments derived from human phage display libraries, Immunotechnology. 4: 71-87; Sheets M D. Amersdorfer P. Finnern R. Sargent P. Lindquist E. Schier R. Hemingsen G. Wong C. Gerhart J C. Marks J D. Lindquist E., (1998) Efficient construction of a large nonimmune phage antibody library: the production of high-affinity human single-chain antibodies to protein antigens (published erratum appears in Proc Natl Acad Sci USA 1999 96: 795), Proc Natl Acad Sci USA 95: 6157-62).

Focused or smart chemical and pharmacophore libraries can be designed with the help of sophisticated strategies involving computational chemistry (e.g., Kundu B. Khare S K. Rastogi S K., (1999) Combinatorial chemistry: polymer supported synthesis of peptide and non-peptide libraries, Progress in Drug Research 53: 89-156) and the use of structure-based ligands using database searching and docking, de novo drug design and estimation of ligand binding affinities (Joseph-McCarthy D., (1999) Computational approaches to structure-based ligand design, Pharmacology & Therapeutics 84: 179-91; Kirkpatrick D L. Watson S. Ulhaq S., (1999) Structure-based drug design: combinatorial chemistry and molecular modeling, Combinatorial Chemistry & High Throughput Screening. 2: 211-21; Eliseev A V. Lehn J M., (1999) Dynamic combinatorial chemistry: evolutionary formation and screening of molecular libraries, Current Topics in Microbiology & Immunology 243: 159-72; Bolger et al., (1991) Methods Enz. 203: 21-45; Martin, (1991) Methods Enz. 203: 587-613; Neidle et al., (1991) Methods Enz. 203: 433-458; U.S. Pat. No. 6,178,384).

Crystallography

After binding compounds have been determined, the orientation of compound bound to target is determined. Preferably this determination involves crystallography on co-crystals of molecular scaffold compounds with target. Most protein crystallographic platforms can preferably be designed to analyze up to about 500 co-complexes of compounds, ligands, or molecular scaffolds bound to protein targets due to the physical parameters of the instruments and convenience of operation. If the number of scaffolds that have binding activity exceeds a number convenient for the application of crystallography methods, the scaffolds can be placed into groups based on having at least one common chemical structure or other desirable characteristics, and representative compounds can be selected from one or more of the classes. Classes can be made with increasingly exacting criteria until a desired number of classes (e.g., 500) is obtained. The classes can be based on chemical structure similarities between molecular scaffolds in the class, e.g., all possess a pyrrole ring, benzene ring, or other chemical feature. Likewise, classes can be based on shape characteristics, e.g., space-filling characteristics.

The co-crystallography analysis can be performed by co-complexing each scaffold with its target at concentrations of the scaffold that showed activity in the screening assay. This co-complexing can be accomplished with the use of low percentage organic solvents with the target molecule and then concentrating the target with each of the scaffolds. In preferred embodiments these solvents are less than 5% organic solvent such as dimethyl sulfoxide (DMSO), ethanol, methanol, or ethylene glycol in water or another aqueous solvent. Each scaffold complexed to the target molecule can then be screened with a suitable number of crystallization screening conditions at both 4 and 20 degrees. In preferred embodiments, about 96 crystallization screening conditions can be performed in order to obtain sufficient information about the co-complexation and crystallization conditions, and the orientation of the scaffold at the binding site of the target molecule. Crystal structures can then be analyzed to determine how the bound scaffold is oriented physically within the binding site or within one or more binding pockets of the molecular family member.

It is desirable to determine the atomic coordinates of the compounds bound to the target proteins in order to determine which is a most suitable scaffold for the protein family. X-ray crystallographic analysis is therefore most preferable for determining the atomic coordinates. Those compounds selected can be further tested with the application of medicinal chemistry. Compounds can be selected for medicinal chemistry testing based on their binding position in the target molecule. For example, when the compound binds at a binding site, the compound's binding position in the binding site of the target molecule can be considered with respect to the chemistry that can be performed on chemically tractable structures or sub-structures of the compound, and how such modifications on the compound might interact with structures or sub-structures on the binding site of the target. Thus, one can explore the binding site of the target and the chemistry of the scaffold in order to make decisions on how to modify the scaffold to arrive at a ligand with higher potency and/or selectivity. This process allows for more direct design of ligands, by utilizing structural and chemical information obtained directly from the co-complex, thereby enabling one to more efficiently and quickly design lead compounds that are likely to lead to beneficial drug products. In various embodiments it may be desirable to perform co-crystallography on all scaffolds that bind, or only those that bind with a particular affinity, for example, only those that bind with high affinity, moderate affinity, low affinity, very low affinity, or extremely low affinity. It may also be advantageous to perform co-crystallography on a selection of scaffolds that bind with any combination of affinities.

Standard X-ray protein diffraction studies such as by using a Rigaku RU-200® (Rigaku, Tokyo, Japan) with an X-ray imaging plate detector or a synchrotron beam-line can be performed on co-crystals and the diffraction data measured on a standard X-ray detector, such as a CCD detector or an X-ray imaging plate detector.

Performing X-ray crystallography on about 200 co-crystals should generally lead to about 50 co-crystals structures, which should provide about 10 scaffolds for validation in chemistry, which should finally result in about 5 selective leads for target molecules.

Virtual Assays

Commercially available software that generates three-dimensional graphical representations of the complexed target and compound from a set of coordinates provided can be used to illustrate and study how a compound is oriented when bound to a target. (e.g., QUANTA®, Accelerys, San Diego, Calif.). Thus, the existence of binding pockets at the binding site of the targets can be particularly useful in the present invention. These binding pockets are revealed by the crystallographic structure determination and show the precise chemical interactions involved in binding the compound to the binding site of the target. The person of ordinary skill will realize that the illustrations can also be used to decide where chemical groups might be added, substituted, modified, or deleted from the scaffold to enhance binding or another desirable effect, by considering where unoccupied space is located in the complex and which chemical substructures might have suitable size and/or charge characteristics to fill it. The person of ordinary skill will also realize that regions within the binding site can be flexible and its properties can change as a result of scaffold binding, and that chemical groups can be specifically targeted to those regions to achieve a desired effect. Specific locations on the molecular scaffold can be considered with reference to where a suitable chemical substructure can be attached and in which conformation, and which site has the most advantageous chemistry available.

An understanding of the forces that bind the compounds to the target proteins reveals which compounds can most advantageously be used as scaffolds, and which properties can most effectively be manipulated in the design of ligands. The person of ordinary skill will realize that steric, ionic, hydrogen bond, and other forces can be considered for their contribution to the maintenance or enhancement of the target-compound complex. Additional data can be obtained with automated computational methods, such as docking and/or Free Energy Perturbations (FEP), to account for other energetic effects such as desolvation penalties. The compounds selected can be used to generate information about the chemical interactions with the target or for elucidating chemical modifications that can enhance selectivity of binding of the compound.

Computer models, such as homology models (i.e., based on a known, experimentally derived structure) can be constructed using data from the co-crystal structures. When the target molecule is a protein or enzyme, preferred co-crystal structures for making homology models contain high sequence identity in the binding site of the protein sequence being modeled, and the proteins will preferentially also be within the same class and/or fold family. Knowledge of conserved residues in active sites of a protein class can be used to select homology models that accurately represent the binding site. Homology models can also be used to map structural information from a surrogate protein where an apo or co-crystal structure exists to the target protein.

Virtual screening methods, such as docking, can also be used to predict the binding configuration and affinity of scaffolds, compounds, and/or combinatorial library members to homology models. Using this data, and carrying out “virtual experiments” using computer software can save substantial resources and allow the person of ordinary skill to make decisions about which compounds can be suitable scaffolds or ligands, without having to actually synthesize the ligand and perform co-crystallization. Decisions thus can be made about which compounds merit actual synthesis and co-crystallization. An understanding of such chemical interactions aids in the discovery and design of drugs that interact more advantageously with target proteins and/or are more selective for one protein family member over others. Thus, applying these principles, compounds with superior properties can be discovered.

Additives that promote co-crystallization can of course be included in the target molecule formulation in order to enhance the formation of co-crystals. In the case of proteins or enzymes, the scaffold to be tested can be added to the protein formulation, which is preferably present at a concentration of approximately 1 mg/ml. The formulation can also contain between 0%-10% (v/v) organic solvent, e.g. DMSO, methanol, ethanol, propane diol, or 1,3 dimethyl propane diol (MPD) or some combination of those organic solvents. Compounds are preferably solubilized in the organic solvent at a concentration of about 10 mM and added to the protein sample at a concentration of about 100 mM. The protein-compound complex is then concentrated to a final concentration of protein of from about 5 to about 20 mg/ml. The complexation and concentration steps can conveniently be performed using a 96-well formatted concentration apparatus (e.g., Amicon Inc., Piscataway, N.J.). Buffers and other reagents present in the formulation being crystallized can contain other components that promote crystallization or are compatible with crystallization conditions, such as DTT, propane diol, glycerol.

The crystallization experiment can be set-up by placing small aliquots of the concentrated protein-compound complex (1 μl) in a 96 well format and sampling under 96 crystallization conditions. (Other screening formats can also be used, e.g., plates with greater than 96 wells.) Crystals can typically be obtained using standard crystallization protocols that can involve the 96 well crystallization plate being placed at different temperatures. Co-crystallization varying factors other than temperature can also be considered for each protein-compound complex if desirable. For example, atmospheric pressure, the presence or absence of light or oxygen, a change in gravity, and many other variables can all be tested. The person of ordinary skill in the art will realize other variables that can advantageously be varied and considered.

Ligand Design and Preparation

The design and preparation of ligands can be performed with or without structural and/or co-crystallization data by considering the chemical structures in common between the active scaffolds of a set. In this process structure-activity hypotheses can be formed and those chemical structures found to be present in a substantial number of the scaffolds, including those that bind with low affinity, can be presumed to have some effect on the binding of the scaffold. This binding can be presumed to induce a desired biochemical effect when it occurs in a biological system (e.g., a treated mammal). New or modified scaffolds or combinatorial libraries derived from scaffolds can be tested to disprove the maximum number of binding and/or structure-activity hypotheses. The remaining hypotheses can then be used to design ligands that achieve a desired binding and biochemical effect.

But in many cases it will be preferred to have co-crystallography data for consideration of how to modify the scaffold to achieve the desired binding effect (e.g., binding at higher affinity or with higher selectivity). Using the case of proteins and enzymes, co-crystallography data shows the binding pocket of the protein with the molecular scaffold bound to the binding site, and it will be apparent that a modification can be made to a chemically tractable group on the scaffold. For example, a small volume of space at a protein binding site or pocket might be filled by modifying the scaffold to include a small chemical group that fills the volume. Filling the void volume can be expected to result in a greater binding affinity, or the loss of undesirable binding to another member of the protein family. Similarly, the co-crystallography data may show that deletion of a chemical group on the scaffold may decrease a hindrance to binding and result in greater binding affinity or specificity.

It can be desirable to take advantage of the presence of a charged chemical group located at the binding site or pocket of the protein. For example, a positively charged group can be complemented with a negatively charged group introduced on the molecular scaffold. This can be expected to increase binding affinity or binding specificity, thereby resulting in a more desirable ligand. In many cases, regions of protein binding sites or pockets are known to vary from one family member to another based on the amino acid differences in those regions. Chemical additions in such regions can result in the creation or elimination of certain interactions (e.g., hydrophobic, electrostatic, or entropic) that allow a compound to be more specific for one protein target over another or to bind with greater affinity, thereby enabling one to synthesize a compound with greater selectivity or affinity for a particular family member. Additionally, certain regions can contain amino acids that are known to be more flexible than others. This often occurs in amino acids contained in loops connecting elements of the secondary structure of the protein, such as alpha helices or beta strands. Additions of chemical moieties can also be directed to these flexible regions in order to increase the likelihood of a specific interaction occurring between the protein target of interest and the compound. Virtual screening methods can also be conducted in silico to assess the effect of chemical additions, subtractions, modifications, and/or substitutions on compounds with respect to members of a protein family or class.

The addition, subtraction, or modification of a chemical structure or sub-structure to a scaffold can be performed with any suitable chemical moiety. For example the following moieties, which are provided by way of example and are not intended to be limiting, can be utilized: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone or N-oxide) or a sulphur atom (e.g., forming a thiol, thione, di-alkylsulfoxide or sulfone) are all examples of moieties that can be utilized.

Additional examples of structures or sub-structures that may be utilized are an aryl optionally substituted with one, two, or three substituents independently selected from the group consisting of alkyl, alkoxy, halogen, trihalomethyl, carboxylate, carboxamide, nitro, and ester moieties; an amine of formula —NX₂X₃, where X₂ and X₃ are independently selected from the group consisting of hydrogen, saturated or unsaturated alkyl, and homocyclic or heterocyclic ring moieties; halogen or trihalomethyl; a ketone of formula —COX₄, where X₄ is selected from the group consisting of alkyl and homocyclic or heterocyclic ring moieties; a carboxylic acid of formula —(X₅)ₙCOOH or ester of formula (X₆)ₙCOOX₇, where X₅, X₆, and X₇ and are independently selected from the group consisting of alkyl and homocyclic or heterocyclic ring moieties and where n is 0 or 1; an alcohol of formula (X₈)ₙOH or an alkoxy moiety of formula —(X₈)ₙOX₉, where X₈ and X₉ are independently selected from the group consisting of saturated or unsaturated alkyl and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1; an amide of formula NHCOX₁₀, where X₁₀ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; SO₂, NX₁₁X₁₂, where X₁₁ and X₁₂ are selected from the group consisting of hydrogen, alkyl, and homocyclic or heterocyclic ring moieties; a homocyclic or heterocyclic ring moiety optionally substituted with one, two, or three substituents independently selected from the group consisting of alkyl, alkoxy, halogen, trihalomethyl, carboxylate, carboxamide, nitro, and ester moieties; an aldehyde of formula —CHO; a sulfone of formula —SO₂X₁₃, where X₁₃ is selected from the group consisting of saturated or unsaturated alkyl and homocyclic or heterocyclic ring moieties; and a nitro of formula —NO₂.

Identification of Attachment Sites on Molecular Scaffolds and Ligands

In addition to the identification and development of ligands for kinases and other enzymes, determination of the orientation of a molecular scaffold or other binding compound in a binding site allows identification of energetically allowed sites for attachment of the binding molecule to another component. For such sites, any free energy change associated with the presence of the attached component should not destablize the binding of the compound to the kinase to an extent that will disrupt the binding. Preferably, the binding energy with the attachment should be at least 4 kcal/mol., more preferably at least 6, 8, 10, 12, 15, or 20 kcal/mol. Preferably, the presence of the attachment at the particular site reduces binding energy by no more than 3, 4, 5, 8, 10, 12, or 15 kcal/mol.

In many cases, suitable attachment sites will be those that are exposed to solvent when the binding compound is bound in the binding site. In some cases, attachment sites can be used that will result in small displacements of a portion of the enzyme without an excessive energetic cost. Exposed sites can be identified in various ways. For example, exposed sites can be identified using a graphic display or 3-dimensional model. In a graphic display, such as a computer display, an image of a compound bound in a binding site can be visually inspected to reveal atoms or groups on the compound that are exposed to solvent and oriented such that attachment at such atom or group would not preclude binding of the enzyme and binding compound. Energetic costs of attachment can be calculated based on changes or distortions that would be caused by the attachment as well as entropic changes.

Many different types of components can be attached. Persons with skill are familiar with the chemistries used for various attachments. Examples of components that can be attached include, without limitation: solid phase components such as beads, plates, chips, and wells; a direct or indirect label; a linker, which may be a traceless linker; among others. Such linkers can themselves be attached to other components, e.g., to solid phase media, labels, and/or binding moieties.

The binding energy of a compound and the effects on binding energy for attaching the molecule to another component can be calculated approximately using any of a variety of available software or by manual calculation. An example is the following:

Calculations were performed to estimate binding energies of different organic molecules to two Kinases: Pim-1 and CDK2. The organic molecules considered included Staurosporine, identified compounds that bind to PIM-1, and several linkers.

Calculated binding energies between protein-ligand complexes were obtained using the FlexX score (an implementation of the Bohm scoring function) within the Tripos software suite. The form for that equation is shown in Eqn. 1 below:
ΔGbind=ΔGtr+ΔGhb+ΔGion+ΔGlipo+ΔGarom+ΔGrot
where: ΔGtr is a constant term that accounts for the overall loss of rotational and translational entropy of the ligand, ΔGhb accounts for hydrogen bonds formed between the ligand and protein, ΔGion accounts for the ionic interactions between the ligand and protein, ΔGlipo accounts for the lipophilic interaction that corresponds to the protein-ligand contact surface, ΔGarom accounts for interactions between aromatic rings in the protein and ligand, and ΔGrot accounts for the entropic penalty of restricting rotatable bonds in the ligand upon binding.

This method estimates the free energy that a lead compound should have to a target protein for which there is a crystal structure, and it accounts for the entropic penalty of flexible linkers. It can therefore be used to estimate the free energy penalty incurred by attaching linkers to molecules being screened and the binding energy that a lead compound should have in order to overcome the free energy penalty of the linker. The method does not account for solvation and the entropic penalty is likely overestimated for cases where the linker is bound to a solid phase through another binding complex, such as a biotin:streptavidin complex.

Co-crystals were aligned by superimposing residues of PIM-1 with corresponding residues in CDK2. The PIM-1 structure used for these calculations was a co-crystal of PIM-1 with a binding compound. The CDK2: Staurosporine co-crystal used was from the Brookhaven database file 1aq1. Hydrogen atoms were added to the proteins and atomic charges were assigned using the AMBER95 parameters within Sybyl. Modifications to the compounds described were made within the Sybyl modeling suite from Tripos.

These calculations indicate that the calculated binding energy for compounds that bind strongly to a given target (such as Staurosporine:CDK2) can be lower than −25 kcal/mol, while the calculated binding affinity for a good scaffold or an unoptimized binding compound can be in the range of −15 to −20. The free energy penalty for attachment to a linker such as the ethylene glycol or hexatriene is estimated as typically being in the range of +5 to +15 kcal/mol.

Linkers

Linkers suitable for use in the invention can be of many different types. Linkers can be selected for particular applications based on factors such as linker chemistry compatible for attachment to a binding compound and to another component utilized in the particular application. Additional factors can include, without limitation, linker length, linker stability, and ability to remove the linker at an appropriate time. Exemplary linkers include, but are not limited to, hexyl, hexatrienyl, ethylene glycol, and peptide linkers. Traceless linkers can also be used, e.g., as described in Plunkett, M. J., and Ellman, J. A., (1995), J. Org. Chem., 60: 6006.

Typical functional groups, that are utilized to link binding compound(s), include, but not limited to, carboxylic acid, amine, hydroxyl, and thiol. (Examples can be found in Solid-supported combinatorial and parallel synthesis of small molecular weight compound libraries; (1998) Tetrahedron organic chemistry series Vol. 17; Pergamon; p 85).

Labels

As indicated above, labels can also be attached to a binding compound or to a linker attached to a binding compound. Such attachment may be direct (attached directly to the binding compound) or indirect (attached to a component that is directly or indirectly attached to the binding compound). Such labels allow detection of the compound either directly or indirectly. Attachment of labels can be performed using conventional chemistries. Labels can include, for example, fluorescent labels, radiolabels, light scattering particles, light absorbent particles, magnetic particles, enzymes, and specific binding agents (e.g., biotin or an antibody target moiety).

Solid Phase Media

Additional examples of components that can be attached directly or indirectly to a binding compound include various solid phase media. Similar to attachment of linkers and labels, attachment to solid phase media can be performed using conventional chemistries. Such solid phase media can include, for example, small components such as beads, nanoparticles, and fibers (e.g., in suspension or in a gel or chromatographic matrix). Likewise, solid phase media can include larger objects such as plates, chips, slides, and tubes. In many cases, the binding compound will be attached in only a portion of such an objects, e.g., in a spot or other local element on a generally flat surface or in a well or portion of a well.

Identification of Biological Agents

The possession of structural information about a protein also provides for the identification of useful biological agents, such as epitopes for development of antibodies, identification of mutation sites expected to affect activity, and identification of attachment sites allowing attachment of the protein to materials such as labels, linkers, peptides, and solid phase media.

Antibodies (Abs) finds multiple applications in a variety of areas including biotechnology, medicine and diagnosis, and indeed they are one of the most powerful tools for life science research. Abs directed against protein antigens can recognize either linear or native three-dimensional (3D) epitopes. The obtention of Abs that recognize 3D epitopes require the use of whole native protein (or of a portion that assumes a native conformation) as immunogens. Unfortunately, this not always a choice due to various technical reasons: for example the native protein is just not available, the protein is toxic, or its is desirable to utilize a high density antigen presentation. In such cases, immunization with peptides is the alternative. Of course, Abs generated in this manner will recognize linear epitopes, and they might or might not recognize the source native protein, but yet they will be useful for standard laboratory applications such as western blots. The selection of peptides to use as immunogens can be accomplished by following particular selection rules and/or use of epitope prediction software.

Though methods to predict antigenic peptides are not infallible, there are several rules that can be followed to determine what peptide fragments from a protein are likely to be antigenic. These rules are also dictated to increase the likelihood that an Ab to a particular peptide will recognize the native protein.
1. Antigenic peptides should be located in solvent accessible regions and contain both hydrophobic and hydrophilic residues.
   For proteins of known 3D structure, solvent accessibility can be determined using a variety of programs such as DSSP, NACESS, or WHATIF, among others.
   If the 3D structure is not known, use any of the following web servers to predict accessibilities: PHD, JPRED, PredAcc (c) ACCpro
2. Preferably select peptides lying in long loops connecting Secondary Structure (SS) motifs, avoiding peptides located in helical regions. This will increase the odds that the Ab recognizes the native protein. Such peptides can, for example, be identified from a crystal structure or crystal structure-based homology model.
   For protein with known 3D coordinates, SS can be obtained from the sequence link of the relevant entry at the Brookhaven data bank. The PDBsum server also offer SS analysis of pdb records.
   When no structure is available secondary structure predictions can be obtained from any of the following servers: PHD, JPRED, PSI-PRED, NNSP, etc
3. When possible, choose peptides that are in the N- and C-terminal region of the protein. Because the N- and C-terminal regions of proteins are usually solvent accessible and unstructured, Abs against those regions are also likely to recognize the native protein.
4. For cell surface glycoproteins, eliminate from initial peptides those containing consensus sites for N-glycosilation.
   N-glycosilation sites can be detected using Scanprosite, or NetNGlyc

In addition, several methods based on various physio-chemical properties of experimental determined epitopes (flexibility, hydrophibility, accessibility) have been published for the prediction of antigenic determinants and can be used. The antizenic index and Preditop are example.

Perhaps the simplest method for the prediction of antigenic determinants is that of Kolaskar and Tongaonkar, which is based on the occurrence of amino acid residues in experimentally determined epitopes. (Kolaskar and Tongaonkar (1990) A semi-empirical method for prediction of antigenic determinants on protein antigens. FEBBS Lett. 276(1-2): 172-174.) The prediction algorithm works as follows:
1. Calculate the average propensity for each overlapping 7-mer and assign the result to the central residue (i+3) of the 7-mer.
2. Calculate the average for the whole protein.
3. (a) If the average for the whole protein is above 1.0 then all residues having average propensity above 1.0 are potentially antigenic.
3. (b) If the average for the whole protein is below 1.0 then all residues having above the average for the whole protein are potentially antigenic.
4. Find 8-mers where all residues are selected by step 3 above (6-mers in the original paper)

The Kolaskar and Tongaonkar method is also available from the GCG package, and it runs using the command egcg.

Crystal structures also allow identification of residues at which mutation is likely to alter the activity of the protein. Such residues include, for example, residues that interact with substrate, conserved active site residues, and residues that are in a region of ordered secondary structure of involved in tertiary interactions. The mutations that are likely to affect activity will vary for different molecular contexts. Mutations in an active site that will affect activity are typically substitutions or deletions that eliminate a charge-charge or hydrogen bonding interaction, or introduce a steric interference. Mutations in secondary structure regions or molecular interaction regions that are likely to affect activity include, for example, substitutions that alter the hydrophobicity/hydrophilicity of a region, or that introduce a sufficient strain in a region near or including the active site so that critical residue(s) in the active site are displaced. Such substitutions and/or deletions and/or insertions are recognized, and the predicted structural and/or energetic effects of mutations can be calculated using conventional software.

IX. Kinase Activity Assays

A number of different assays for kinase activity can be utilized for assaying for active modulators and/or determining specificity of a modulator for a particular kinase or group or kinases. In addition to the assays mentioned below, one of ordinary skill in the art will know of other assays that can be utilized and can modify an assay for a particular application.

An assay for kinase activity that can be used for kinases, e.g., PIM-1, can be performed according to the following procedure using purified kinase using myelin basic protein (MBP) as substrate. An exemplary assay can use the following materials: MBP (M−1891, Sigma); Kinase buffer (KB=HEPES 50 mM, pH7.2, MgCl₂:MnCl₂ (200 μM:200 μM); ATP (γ-³³P):NEG602H (10 mCi/mL)(Perkin-Elmer); ATP as 100 mM stock in kinase buffer; EDTA as 100 mM stock solution.

Coat scintillation plate suitable for radioactivity counting (e.g., FlashPlate from Perkin-Elmer, such as the SMP200(basic)) with kinase+MBP mix (final 100 ng+300 ng/well) at 90-EL/well in kinase buffer. Add compounds at 1 EL/well from 10 mM stock in DMSO. Positive control wells are added with 1 μL of DMSO. Negative control wells are added with 2 μL of EDTA stock solution. ATP solution (10 μL) is added to each well to provide a final concentration of cold ATP is 2 μM, and 50 nCi ATPγ[³³P]. The plate is shaken briefly, and a count is taken to initiate count (IC) using an apparatus adapted for counting with the plate selected, e.g., Perkin-Elmer Trilux. Store the plate at 37° C. for 4 hrs, then count again to provide final count (FC).

Net 33P incorporation (NI) is calculated as: NI=FC−IC.

The effect of the present of a test compound can then be calculated as the percent of the positive control as: % PC=[(NI−NC)/(PC−NC)]×100, where NC is the net incorporation for the negative control, and PC is the net incorporation for the positive control.

As indicated above, other assays can also be readily used. For example, kinase activity can be measured on standard polystyrene plates, using biotinylated MBP and ATPγ[³³P] and with Streptavidin-coated SPA (scintillation proximity) beads providing the signal.

Additional alternative assays can employ phospho-specific antibodies as detection reagents with biotinylated peptides as substrates for the kinase. This sort of assay can be formatted either in a fluorescence resonance energy transfer (FRET) format, or using an AlphaScreen (amplified luminescent proximity homogeneous assay) format by varying the donor and acceptor reagents that are attached to streptavidin or the phosphor-specific antibody.

X. Organic Synthetic Techniques

The versatility of computer-based modulator design and identification lies in the diversity of structures screened by the computer programs. The computer programs can search databases that contain very large numbers of molecules and can modify modulators already complexed with the enzyme with a wide variety of chemical functional groups. A consequence of this chemical diversity is that a potential modulator of kinase function may take a chemical form that is not predictable. A wide array of organic synthetic techniques exist in the art to meet the challenge of constructing these potential modulators. Many of these organic synthetic methods are described in detail in standard reference sources utilized by those skilled in the art. One example of such a reference is March, 1994, Advanced Organic Chemistry: Reactions, Mechanisms and Structure, New York, McGraw Hill. Thus, the techniques useful to synthesize a potential modulator of kinase function identified by computer-based methods are readily available to those skilled in the art of organic chemical synthesis.

XI. Isomers, Prodrugs, and Active Metabolites

The present compounds are described herein with generic formulas and specific compounds. In addition, the present compounds may exist in a number of different forms or derivatives, all within the scope of the present invention. These include, for example, tautomers, enantiomers, stereoisomers, racemic mixtures, regioisomers, salts, prodrugs (e.g., carboxylic acid esters), solvated forms, different crystal forms or polymorphs, and active metabolites.

A. Tautomers, Stereoisomers, Regioisomers, and Solvated Forms

It is understood that certain compounds may exhibit tautomerism. In such cases, the formula drawings within this specification expressly depict only one of the possible tautomeric forms. It is therefore to be understood that within the invention the formulas are intended to represent any tautomeric form of the depicted compounds and are not to be limited merely to the specific tautomeric form depicted by the formula drawings.

Likewise, some of the present compounds may contain one or more chiral centers, and therefore, may exist in two or more stereoisomeric forms. Thus, such compounds may be present as single stereoisomers (i.e., essentially free of other stereoisomers), racemates, and/or mixtures of enantiomers and/or diastereomers. All such single stereoisomers, racemates and mixtures thereof are intended to be within the scope of the present invention. Unless specified to the contrary, all such steroisomeric forms are included within the formulas provided herein.

In certain embodiments, a chiral compound of the present invention is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In certain embodiments, the compound is present in optically pure form.

For compounds is which synthesis involves addition of a single group at a double bond, particularly a carbon-carbon double bond, the addition may occur at either of the double bond-linked atoms. For such compounds, the present invention includes both such regioisomers.

Additionally, the formulas are intended to cover solvated as well as unsolvated forms of the identified structures. For example, the indicated structures include both both hydrated and non-hydrated forms. Other examples of solvates include the structures in combination with isopropanol, ethanol, methanol, DMSO, ethyl acetate, acetic acid, or ethanolamine.

B. Prodrugs and Metabolites

In addition to the present formulas and compounds described herein, the invention also includes prodrugs (generally pharmaceutically acceptable prodrugs), active metabolic derivatives (active metabolites), and their pharmaceutically acceptable salts.

In this context, prodrugs are compounds that may be converted under physiological conditions or by solvolysis to the specified compound or to a pharmaceutically acceptable salt of such a compound. A common example is an alkyl ester of a carboxylic acid.

As described in The Practice of Medicinal Chemistry, Ch. 31-32 (Ed. Wermuth, Academic Press, San Diego, Calif., 2001), prodrugs can be conceptually divided into two non-exclusive categories, bioprecursor prodrugs and carrier prodrugs. Generally, bioprecursor prodrugs are compounds are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

Oxidative reactions, such as oxidation of alcohol, carbonyl, and acid functions, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-delakylation, oxidative Q- and S-delakylation, oxidative deamination, as well as other oxidative reactions.

Reductive reactions, such as reduction of carbonyl groups, reduction of alcoholic groups and carbon-carbon double bonds, reduction of nitrogen-containing functions groups, and other reduction reactions.

Reactions without change in the state of oxidation, such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety in intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent publ. 20040077595, application Ser. No. 10/656,838, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, The Practice of Medicinal Chemistry, Ch. 31-32, Ed. Wermuth, Academic Press, San Diego, Calif., 2001.

Prodrugs may proceed from prodrug form to active form in a single step or may have one or more intermediate forms which may themselves have activity or may be inactive.

Metabolites, e.g., active metabolites overlap with prodrugs as described above, e.g., bioprecursor prodrugs. Thus, such metabolites are pharmacologically active compounds or compounds that further metabolize to pharmacologically active compounds that are derivatives resulting from metabolic process in the body of a subject or patient. Of these, active metabolites are such pharmacologically active derivative compounds. For prodrugs, the prodrug compounds is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40: 2011-2016; Shan et al., J Pharm Sci 86: 756-757; Bagshawe, 1995, Drug Dev Res 34: 220-230; Wermuth, The Practice of Medicinal Chemistry, Ch. 31-32, Academic Press, San Diego, Calif., 2001.

C. Pharmaceutically Acceptable Salts

Compounds can be formulated as or be in the form of pharmaceutically acceptable salts. Pharmaceutically acceptable salts are non-toxic salts in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound is dissolved in a suitable solvent, such as an aqueous or aqueous-alcohol in solution containing the appropriate acid and then isolated by evaporating the solution. In another example, a salt is prepared by reacting the free base and acid in an organic solvent.

Thus, for example, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Unless specified to the contrary, specification of a compound herein includes pharmaceutically acceptable salts of such compound.

D. Polymorphic Forms

In the case of agents that are solids, it is understood by those skilled in the art that the compounds and salts may exist in different crystal or polymorphic forms, all of which are intended to be within the scope of the present invention and specified formulas.

XII. Administration

The methods and compounds will typically be used in therapy for human patients. However, they may also be used to treat similar or identical diseases in other vertebrates such as other primates, sports animals, and pets such as horses, dogs and cats.

Suitable dosage forms, in part, depend upon the use or the route of administration, for example, oral, transdermal, transmucosal, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington's Pharmaceutical Sciences, 18^{th} ed., Mack Publishing Co., Easton, Pa., 1990 (hereby incorporated by reference herein).

Carriers or excipients can be used to produce pharmaceutical compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, or transdermal. Oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The amounts of various compound to be administered can be determined by standard procedures taking into account factors such as the compound IC₅₀, the biological half-life of the compound, the age, size, and weight of the patient, and the disorder associated with the patient. The importance of these and other factors are well known to those of ordinary skill in the art. Generally, a dose will be between about 0.01 and 50 mg/kg, preferably 0.1 and 20 mg/kg of the patient being treated. Multiple doses may be used.

XIII. Manipulation of Kinase Coding Sequences

Through the availability of the coding sequences for many different kinases, any of a variety of different molecular techniques can be performed as desired, e.g., cloning, construction of recombinant sequences, production and purification of recombinant protein, introduction of particular kinase sequences into other organisms, and the like.

Techniques for the manipulation of nucleic acids, such as, e.g., subcloning, labeling probes (e.g., random-primer labeling using Klenow polymerase, nick translation, amplification), sequencing, hybridization and the like are well disclosed in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, Tijssen, ed. Elsevier, N.Y. (1993). Nucleic acid sequences can be amplified as necessary for further use using amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1; 29(11): E54-E54; Hafner et al., Biotechniques 2001 April; 30(4): 852-6, 858, 860 passim; Zhong et al., Biotechniques 2001 April; 30(4): 852-6, 858, 860 passim.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be performed by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15: 333-335; yeast artificial chromosomes (YAC); bacterial artificial chromosomes (BAC); P1 artificial chromosomes, see, e.g., Woon (1998) Genomics 50: 306-316; P1-derived vectors (PACs), see, e.g., Kern (1997) Biotechniques 23: 120-124; cosmids, recombinant viruses, phages or plasmids.

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The nucleic acids of the invention can also be provided in expression vectors and cloning vehicles, e.g., sequences encoding the polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are disclosed, e.g., U.S. Pat. No. 5,426,039. To facilitate cloning of amplified sequences, restriction enzyme sites can be “built into” a PCR primer pair. Vectors may be introduced into a genome or into the cytoplasm or a nucleus of a cell and expressed by a variety of conventional techniques, well described in the scientific and patent literature. See, e.g., Roberts (1987) Nature 328: 731; Schneider (1995) Protein Expr. Purif 6435: 10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15: 866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher (1992) J. Virol. 66: 2731-2739; Johann (1992) J. Virol. 66: 1635-1640). Adeno-associated virus (AAV)-based vectors can be used to transduce cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada (1996) Gene Ther. 3: 957-964.

The present invention also relates to fusion proteins, and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34: 1787-1797; Dobeli (1998) Protein Expr. Purif 12: 404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well disclosed in the scientific and patent literature, see e.g., Kroll (1993) DNA Cell. Biol. 12: 441-53.

The nucleic acids and polypeptides of the invention can be bound to a solid support, e.g., for use in screening and diagnostic methods. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of molecules to a solid support can be direct (i.e., the molecule contacts the solid support) or indirect (a “linker” is bound to the support and the molecule of interest binds to this linker). Molecules can be immobilized either covalently (e.g., utilizing single reactive thiol groups of cysteine residues (see, e.g., Colliuod (1993) Bioconjugate Chem. 4: 528-536) or non-covalently but specifically (e.g., via immobilized antibodies (see, e.g., Schuhmann (1991) Adv. Mater. 3: 388-391; Lu (1995) Anal. Chem. 67: 83-87; the biotin/strepavidin system (see, e.g., Iwane (1997) Biophys. Biochem. Res. Comm. 230: 76-80); metal chelating, e.g., Langmuir-Blodgett films (see, e.g., Ng (1995) Langmuir 11: 4048-55); metal-chelating self-assembled monolayers (see, e.g., Sigal (1996) Anal. Chem. 68: 490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl(4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can also be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon (1989) Nature 377: 525-531 (1989).

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface. In practicing the methods of the invention, any known array and/or method of making and using arrays can be incorporated in whole or in part, or variations thereof, as disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8: R171-R174; Schummer (1997) Biotechniques 23: 1087-1092; Kern (1997) Biotechniques 23: 120-124; Solinas-Toldo (1997) Genes, Chromosomes & Cancer 20: 399-407; Bowtell (1999) Nature Genetics Supp. 21: 25-32. See also published U.S. patent applications Nos. 20010018642; 20010019827; 20010016322; 20010014449; 20010014448; 20010012537; 20010008765.

Host Cells and Transformed Cells Comprising Kinase Sequences

As indicated above, availability of kinase coding sequences also allows provision of a transformed cell comprising a kinase nucleic acid sequence, e.g., a sequence encoding a kinase polypeptide, or a vector. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

Vectors may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

For transient expression in mammalian cells, cDNA encoding a polypeptide of interest may be incorporated into a mammalian expression vector, e.g. pcDNA1, which is available commercially from Invitrogen Corporation (San Diego, Calif., U.S.A.; catalogue number V490-20). This is a multifunctional 4.2 kb plasmid vector designed for cDNA expression in eukaryotic systems, and cDNA analysis in prokaryotes, incorporated on the vector are the CMV promoter and enhancer, splice segment and polyadenylation signal, an SV40 and Polyoma virus origin of replication, and M13 origin to rescue single strand DNA for sequencing and mutagenesis, Sp6 and T7 RNA promoters for the production of sense and anti-sense RNA transcripts and a Col E1-like high copy plasmid origin. A polylinker is located appropriately downstream of the CMV promoter (and 3′ of the T7 promoter).

The cDNA insert may be first released from the above phagemid incorporated at appropriate restriction sites in the pcDNAI polylinker. Sequencing across the junctions may be performed to confirm proper insert orientation in pcDNAI. The resulting plasmid may then be introduced for transient expression into a selected mammalian cell host, for example, the monkey-derived, fibroblast like cells of the COS-1 lineage (available from the American Type Culture Collection, Rockville, Md. as ATCC CRL 1650).

For transient expression of the protein-encoding DNA, for example, COS-1 cells may be transfected with approximately 8 μg DNA per 10⁶ COS cells, by DEAE-mediated DNA transfection and treated with chloroquine according to the procedures described by Sambrook et al, Molecular Cloning: A Laboratory Manual, 1989, Cold Spring Harbor Laboratory Press, Cold Spring Harbor N.Y, pp. 16.30-16.37. An exemplary method is as follows. Briefly, COS-1 cells are plated at a density of 5×10⁶ cells/dish and then grown for 24 hours in FBS-supplemented DMEM/F12 medium. Medium is then removed and cells are washed in PBS and then in medium. A transfection solution containing DEAE dextran (0.4 mg/ml), 100 μM chloroquine, 10% NuSerum, DNA (0.4 mg/ml) in DMEM/F 12 medium is then applied on the cells 10 ml volume. After incubation for 3 hours at 37° C., cells are washed in PBS and medium as just described and then shocked for 1 minute with 10% DMSO in DMEM/F12 medium. Cells are allowed to grow for 2-3 days in 10% FBS-supplemented medium, and at the end of incubation dishes are placed on ice, washed with ice cold PBS and then removed by scraping. Cells are then harvested by centrifugation at 1000 rpm for 10 minutes and the cellular pellet is frozen in liquid nitrogen, for subsequent use in protein expression. Northern blot analysis of a thawed aliquot of frozen cells may be used to confirm expression of receptor-encoding cDNA in cells under storage.

In a like manner, stably transfected cell lines can also prepared, for example, using two different cell types as host: CHO K1 and CHO Pro5. To construct these cell lines, cDNA coding for the relevant protein may be incorporated into the mammalian expression vector pRC/CMV (Invitrogen), which enables stable expression. Insertion at this site places the cDNA under the expression control of the cytomegalovirus promoter and upstream of the polyadenylation site and terminator of the bovine growth hormone gene, and into a vector background comprising the neomycin resistance gene (driven by the SV40 early promoter) as selectable marker.

An exemplary protocol to introduce plasmids constructed as described above is as follows. The host CHO cells are first seeded at a density of 5×10⁵ in 10% FBS-supplemented MEM medium. After growth for 24 hours, fresh medium is added to the plates and three hours later, the cells are transfected using the calcium phosphate-DNA co-precipitation procedure (Sambrook et al, supra). Briefly, 3 μg of DNA is mixed and incubated with buffered calcium solution for 10 minutes at room temperature. An equal volume of buffered phosphate solution is added and the suspension is incubated for 15 minutes at room temperature. Next, the incubated suspension is applied to the cells for 4 hours, removed and cells were shocked with medium containing 15% glycerol. Three minutes later, cells are washed with medium and incubated for 24 hours at normal growth conditions. Cells resistant to neomycin are selected in 10% FBS-supplemented alpha-MEM medium containing G418 (1 mg/ml). Individual colonies of G418-resistant cells are isolated about 2-3 weeks later, clonally selected and then propagated for assay purposes.

### EXAMPLES

### Example 1

### Cloning of PIM-1

The PIM-1 DNA encoding amino acids 1-313 and 29-313 were amplified from human brain cDNA (Clonetech) by PCR protocols and cloned into a modified pET 29 vector (Novagen) between NdeI and SalI restriction enzyme sites. The amino acid sequences of the cloned DNA were confirmed by DNA sequencing and the expressed proteins contain a hexa-histidine sequence at the C terminus. The protein was expressed in E. coli BL21(DE3)pLysS (Novagen). The bacteria were grown at 22° C. in Terrific broth to 1-1.2 OD600 and protein was induced by 1 mM IPTG for 16-18 h. The bacterial pellet was collected by centrifugation and stored at −70° C. until used for protein purification. PIM-2 and PIM-3 are cloned similarly.

### Example 2

### Purification of PIM-1

The bacterial pellet of approximately 250-300 g (usually from 16 L) expressing PIM-1 kinase domain (29-313) was suspended in 0.6 L of Lysis buffer (0.1 M potassium phosphate buffer, pH 8.0, 10% glycerol, 1 mM PMSF) and the cells were lysed in a French Pressure cell at 20,000 psi. The cell extract was clarified at 17,000 rpm in a Sorval SA 600 rotor for 1 h. The supernatant was re-centrifuged at 17000 rpm for another extra hour. The clear supernatant was added with imidazole (pH 8.0) to 5 mM and 2 ml of cobalt beads (50% slurry) to each 40 ml cell extract. The beads were mixed at 4° C. for 3-4 h on a nutator. The cobalt beads were recovered by centrifugation at 4000 rpm for 5 min. The pelleted beads were washed several times with lysis buffer and the beads were packed on a Byroad disposable column. The bound protein was eluted with 3-4 column volumes of 0.1 M imidazole followed by 0.25 M imidazole prepared in lysis buffer. The eluted protein was analyzed by SDS gel electrophoresis for purity and yield.

The eluted protein from cobalt beads was concentrated by Centriprep-10 (Amicon) and separated on Pharmacia Superdex 200 column (16/60) in low salt buffer (25 mM Tris-HCl, pH 8.0, 150 mM NaCl, 14 mM beta mercaptoethanol). The peak fractions containing PIM-1 kinase was further purified on a Pharmacia Source Q column (10/10) in 20 mM Tris-HCl pH 7.5 and 14 mM beta mercaptoethanol using a NaCl gradient in an AKTA-FPLC (Pharmacia). The PIM-1 kinase eluted approximately at 0.2 M NaCl gradient. The peak fractions were analyzed by SDS gel electrophoresis and were pooled and concentrated by Centriprep 10. The concentrated PIM-1 protein (usually 50-60 A280/ml) was aliquoted into many tubes (60 ul), flash frozen in liquid nitrogen and stored at −70° C. until used for crystallization. The frozen PIM-1 kinase still retained kinase activity as concluded from activity assays. PIM-2 and PIM-3 can be purified in the same way with small adjustments to conditions, e.g., elution conditions.

### Example 3

### Variants and Derivatives of PIM-1

In mouse, PIM-1 is expressed as two forms of 44 kDa and 33 kDa. The p44 kDa PIM-1 is encoded by the same gene as p33 kDa PIM-1 but the translation is initiated at an upstream CUG codon (Saris C J, Domen J, and Berns A. (1991) The PIM-1 oncogene encodes two related protein-serine/threonine kinases by alternative initiation at AUG and CUG. EMBO J. 10: 655-664.) This results in expression of p44 PIM-1 having a unique 11 kDa N terminal extension that is followed by the p33 PIM-1 sequence. The p33 kDa PIM-1 contains almost the entire kinase domain and both p33 and p44 kDa have comparable kinase activity and both can prevent apoptosis (Lilly M, Sandholm J, Cooper J J, Koskinen P J, and Kraft A. (1999) The PIM-1 serine kinase prolongs survival and inhibits apoptosis-related mitochondrial dysfunction in part through a bcl-2-dependent pathway. Oncogene., 18: 4022-4031). CD40 engagement caused significant increase in the levels of both 33 and 44 kDa forms of PIM1 in cytoplasmic extracts of WEHI-231 cells (Zhu N, Ramirez L M, Lee R L, Magnuson N S, Bishop G A, and Gold M R. (2002) CD40 signaling in B cells regulates the expression of the PIM-1 kinase via the NF-kappa B pathway. J. Immunol. 168: 744-754). Recently it has been shown that the p33 kDa form was more strongly associated with Socs-1 than the p44 kDa form (Chen X P, Losman J A, Cowan S, Donahue E, Fay S, Vuong B Q, Nawijn M C, Capece D, Cohan V L, Rothman P. (2002) PIM serine/threonine kinases regulate the stability of Socs-1 protein. Proc Natl Acad Sci USA., 99: 2175-2180).

There are no reports of PIM-1 existing in more than one form in human. Analysis of PIM-1 gene sequence reveals that the presence of in-frame stop codons block synthesis of proteins with N terminal extensions. However, the human PIM-2 gene contains no in-frame stop codon, based on the reported DNA sequence. Therefore, alternate initiation at an upstream start codon is possible. We have expressed the PIM-2 kinase domain in E. coli and purified the protein by the same methods as described for PIM-1 kinase.

### Example 4

### Crystallization of PIM-1

PIM-1 Protein Crystal Growth:

All materials were purchased through Hampton Research, Inc. (Laguna Niguel, Calif.) unless otherwise noted. PIM-1 protein @ 7 and 14 mg/ml was screened against Hampton Crystal Screen 1 and 2 kits (HS1 and HS2) and yielded successful crystals growing in at least 10 conditions from HS1 alone. Crystals were grown initially using sitting drops against the Hampton screening conditions set in Greiner 96 well CrystalQuick crystallization plates with 100 ul reservoir and 1 ul protein+1 ul reservoir added per platform (1 of 3 available). Conditions from Hampton Screen 1 yielded obvious protein crystals in conditions: #2, 7, 14, 17, 23, 25, 29, 36, 44, and 49. These crystals were grown at 4° C., and grew in size to varying dimensions, all hexagonal rod shaped and hardy.

Crystals of larger dimensions, 100 uM wide×400 uM long, were then grown in larger drop volumes and in larger dimension plates. Refined grids were performed with both hanging and sitting drop methods in VDX plates (cat. # HR3-140) or CrysChem plates (cat. # HR3-160). There appeared to be no obvious difference of crystal size or quality between the two methods, but there was a preference to use hanging drops to facilitate mounting procedures.

We proceeded with refining conditions by gridding 4 independent reservoir conditions initially obtained from the screening kits.
1) HS1 # 17 was optimized to 0.2 M LiCl, 0.1 M Tris pH 8.5 and 5%-15% Polyethylene glycol 4000;
2) HS1 # 25 was optimized to 0.4 M-0.9 M Sodium Acetate trihydrate pH 6.5 and 0.1 M Imidazole;
3) HS1 # 29 was optimized to 0.2M-0.7 M Sodium Potassium tartrate and 0.1 M MES buffer pH 6.5;
4) HS1 # 44 was optimized to 0.25 M Magnesium formate.

These optimized conditions produced crystals with the most consistent size and quality of appearance. Conditions were further evaluated by x-ray diffraction analysis of the resulting protein crystals, and keeping in mind the utility for forming compound co-crystals in these conditions as well (ie. salt composition and concentration effects are important to develop suitable compound solubility in the crystallization experiments). Native crystals grew as rods in many drops to large dimensions of approximately 100 um wide and 500 um long.

Seleno Methionine Labeled PIM-1 Protein Crystal Growth.

Se-Met labeled PIM protein was expressed and purified as described by Hendrickson, W. A., and Ogata, C. M. (1997) “Phase determination from multiwavelength anomalous diffraction measurements, Methods Enzymol., 276, 494-523, and Hendrickson, W. A., Horton, J. R., and LeMaster, D. M. (1990) “Selenomethionyl proteins produced for analysis by multiwavelength anomalous diffraction (MAD): a vehicle for direct determination of three-dimentional structure, EMBO J., 9, 1665-1672. This preparation appeared to be less soluble as evidenced by more pronounced nucleation within the screen drops and due to the hydrophobic nature of Se labeled proteins. Crystals grew small and in showers compared to the previously evaluated similar drop conditions that the native protein grew well in. Upon finer gridding, 20 μm wide×100 μm long crystals were obtained in condition HS1 # 17 optimized at 0.2 M LiCl, 0.1 M Tris pH 8.5 and 5%-15% PEG 4000. These crystals and all others were carefully mounted in 50-100 uM nylon loops on copper stem magnetic bases that were flash frozen in liquid nitrogen in appropriate cryogenic buffer and taken to the Lawerence Berkeley Lab synchrotron, the Advanced Light Source (ALS) beamline 8.3.1.

PIM-1 protein/Molecular Scaffolds Co-Crystal Growth:

In order to add compounds to PIM-1 protein, compounds were added directly from their DMSO stocks (20-200 mM) into the protein solution at high concentration. The procedure involved adding the DMSO stocks containing compound as a thin layer to the wall of the 1.5 ml eppendorf tube that contains the protein. The solution was then gently rolled over the wall of the tube until the compound was in the protein solution. The final concentration of compounds in the PIM-1 solution usually achieved was between 0.5 and 1 mM with DMSO concentrations less than 2% being added. The solutions were then set-up in trays immediately as previously described.

PIM-1/Compound Co-Crystal Screening in HS1:

Two conditions for crystal growth have resulted in the best results with PIM-1 protein and added compounds. The optimized Na-K tartrate and Na-acetate tetrahydrate solutions listed above. Crystals varied greatly in size but data has been collected on various crystals that are between 20 uM and 100 uM in width. These crystals were typically several hundred microns long and some required manipulation as well as being broken to facilitate mounting procedures into loops. Interestingly, some crystals that were grown in the presence of colored compounds were also colored the same way.

### Example 5

### Diffraction Analysis of PIM-1

Crystals were first determined to diffract on a Rigaku RU-200 rotating copper anode x-ray source equipped with Yale focusing optics and an R-AXIS 2C imaging plate system. A crystal grown in the optimized condition HS1 # 17 (DY plate Dec. 14, 2001) was used to conduct initial diffraction experiments.

After x-ray diffraction was initially determined as described above, large native protein crystals grown in Mg-Formate (DY plate) and were frozen in cryoprotectant by submersion in liquid nitrogen and then tested for diffraction at ALS beamline 8.3.1. Data was originally collected, indexed and reduced using Mosflm. The spacegroup was determined to be P65.

We have collected 3 native data sets, the highest resolution obtained with good statistics after merging is to 2.0 angstroms.

We have collected a MAD data set on the Se-Met labeled PIM-1 crystal using the experimentally determined 12668 eV peak and 11000 eV remote for selenium to 3.2 angstroms. Subsequently a 2.6 angstrom Se peak data set was collected at the experimentally determined peak of 12668 eV radiation.

We have collected more than 50 PIM-1/binding compound co-crystal data sets. All data was indexed and reduced as indicated in the computational crystallographic work that follows.

PIM-1 Structure Determination and Refinement

Data Set: Native, Resolution: 2.13

The primary structure determination was carried out using Molecular Replacement method with programs
EPMR (Public domain)
AmoRe (from CCP4))
And a homology model of PIM-1 based on the protein Phosphorylase Kinase (PDB ID: 1PHK—Owen et al., 1995, Structure 3: 467)

The molecular replacement was carried out in all of the P6 space groups (P61, P62, . . . P65). The best solution was obtained in P65.

The molecular replacement solution was improved by several rounds of the cycles of
Model Building in 0 (from DatOno AB)
Annealing in CNX (from Accelerys)
SigmaA weighting and Solvent Flattening the resultant map with DM (from CCP4)

The statistics at the end of these cycles were R˜36%.

Data Set: SeMet (2 Wavelengths), Resolution: 3.3

The MAD phased data (with SOLVE (from Los Alamos National Laboratory)) helped improve the model in the refinement with REFMAC (from CCP4).

Data Set: SeMet (1 Wavelength), Resolution: 2.6

Further improvement of the model was obtained using SAD Phasing with SOLVE and subsequent improvement with RESOLVE produced an excellent map into which the PIM1 model could be rebuilt completely.

The newly built model refined with CNX/Anneal and then with CCP4/Refmac to give R=27.7% and Rfree=31.9%

Data Set: Native, Resolution: 2.1

The above model has been further refined against the native data with CCP4/Refmac, giving R=22.1%, Rfree=24.2%.

### Example 6

### Co-Crystal Structures

Exemplary co-crystal structures have been determined for 7 compounds with PIM-1, using methods as generally described above. Those co-crystals are the following (the number indicates the compound id and the compound source is provided in parentheses):
PIM1_{—}5321980 (Chembridge)
PIM1_RB00137 (Maybridge)
PIM1_{—}5264241 (Chembridge)
PIM1_RJF00907 (Maybridge)
PIM1_{—}5140994 (Chembridge)
PIM1_{—}5108305 (Chembridge)
PIM1_BTB02713 (Maybridge)

### Example 7

### PIM Binding Assays

Such binding assays can be performed in a variety of ways, including a variety of ways known in the art. For example, competitive binding to PIM-1 can be measured on Nickel-FlashPlates, using His-tagged PIM-1 (100 ng) and ATPγ[³⁵S] (˜10 nCi). As compound is added, the signal decreases, since less ATPγ[³⁵S] is bound to PIM1 which is proximal to the scintillant in the FlashPlate. The binding assay can be performed by the addition of compound (10 μl; 20 mM) to PIM-1 protein (90 10 μl) followed by the addition of ATPγ[³⁵S] and incubating for 1 hr at 37° C. The radioactivity is measured through scintillation counting in Trilus (Perkin-Elmer).

Alternatively, any method which can measure binding of a ligand to the ATP-binding site can be used. For example, a fluorescent ligand can be used. When bound to PIM1, the emitted fluorescence is polarized. Once displaced by inhibitor binding, the polarization decreases.

Determination of IC50 for compounds by competitive binding assays. (Note that K_{I} is the dissociation constant for inhibitor binding; K_{D} is the dissociation constant for substrate binding.) For this system, the IC50, inhibitor binding constant and substrate binding constant can be interrelated according to the following formula:

$$\text{When using radiolabeled substrate} {K}_{I} = \frac{IC50}{1 + [ {L}^{*} ] / {K}_{D}} .$$

the IC50˜K_{I} when there is a small amount of labeled substrate.

### Example 8

### PIM Activity Assays

Inhibitory or exhitory activity of compounds binding to PIM-1 was determined using the kinase activity assay described in the detailed description.

Exemplary compounds within Formula I, Formula II, and Formula III were assayed for inhibitory activity with PIM-1. The ability to develop ligands is illustrated by 2 compounds from the quinolinone molecular scaffold group (Formula III). A compound with R1, R2, R3, R4, R5, and R6=H, had 100% inhibition of PIM-1 at 200 μM concentration, while a compound with R1=phenyl group, R2, R3, R5, and R7=H, and R4=OCF₃, had only 3% inhibition of PIM-1 at 200 μM.

### Example 9

### Synthesis of the Compounds of Formula I

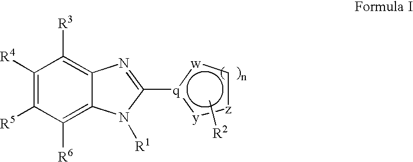

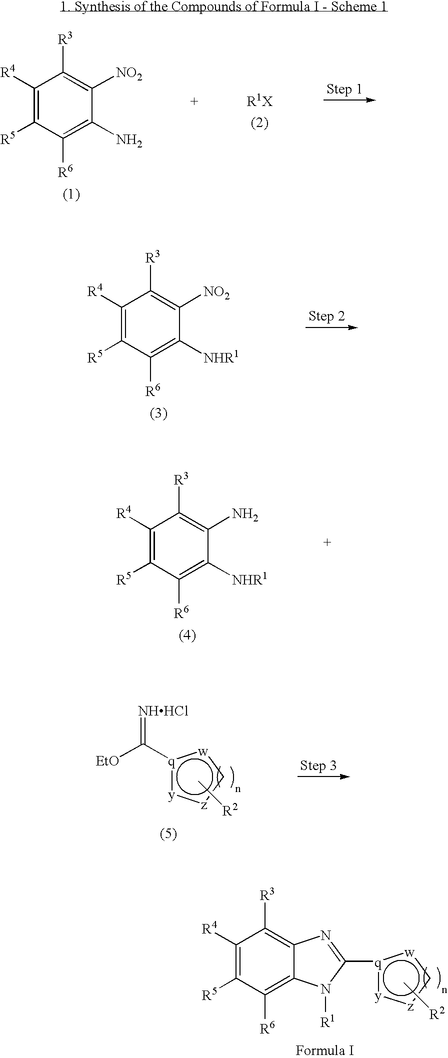

The 2-arylbenzimidazoles derivatives, represented by Formula I, can be prepared as shown in Scheme 1.

Step 1 Preparation of Formula (3)

Compound of formula (3) was prepared by a reaction of a compound of formula (1) with an alkyl halide of formula (2)(e.g. X=Br) in an inert solvent (e.g. DMF), in the presence of a base (e.g. NaH) and heated near 100° C. for 24-36 h. Compound of formula (3) was purified by column chromatography.

Step 2 Preparation of Formula (4)

Compound of formula (4) was prepared by a reaction of a compound of formula (3) with a reducing agent (e.g. SnCl₂) in a polar solvent (e.g. EtOH) and heated near 100° C. for 5-12 h. When the reaction is completed, the product of formula (4) is isolated by conventional means (e.g. aqueous base workup).

Step 3 Preparation of Formula I

Compound of formula I was prepared by the reaction of the compound of formula (4) with an imidate of formula (5) in a polar solvent (e.g. EtOH) and heated near 80° C. for 12-18 h. Compound of formula I was purified by column chromatography.

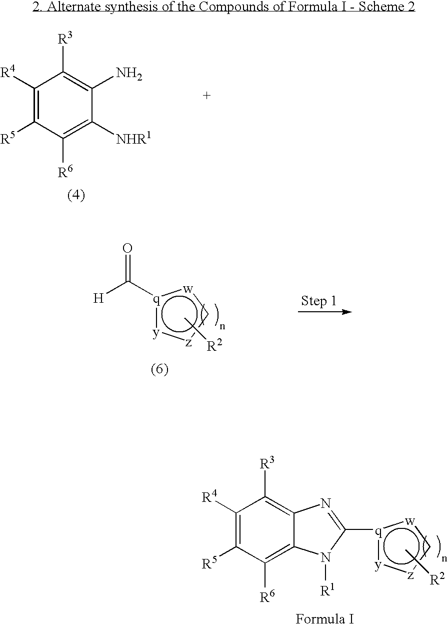

Step 1 Preparation of Formula I

Compound of formula I was prepared by the reaction of the compound of formula (4) with an aldehyde of formula (6) in a polar solvent (e.g. EtOH) and heated near 80° C. for 12-22 h. Compound of formula I was purified by column chromatography.

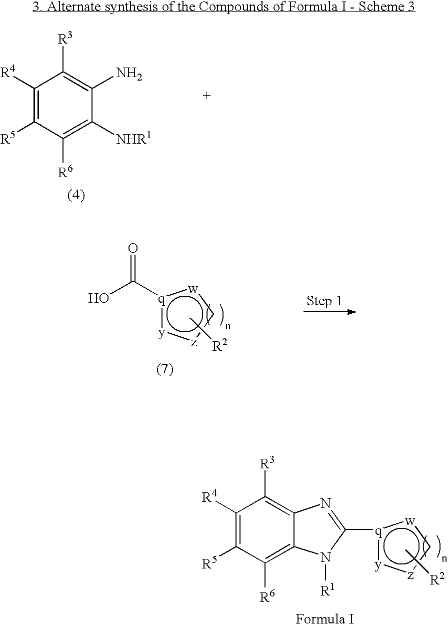

Step 1 Preparation of Formula I

Compound of formula I was prepared by the reaction of the compound of formula (4) with an aldehyde of formula (7) in an inorganic acid (e.g. polyphosphoric acid) and heating near 190-220° C. for 5-12 h. Compound of formula I was purified by column chromatography.

### Example 10

### Synthesis of the Compounds of Formula II

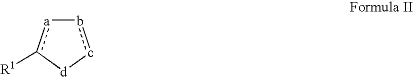

Synthesis schemes for exemplary groups of compounds within Formula II are provided below. Persons skilled in chemical synthesis will readily understand how to synthesize additional compounds within Formula II.

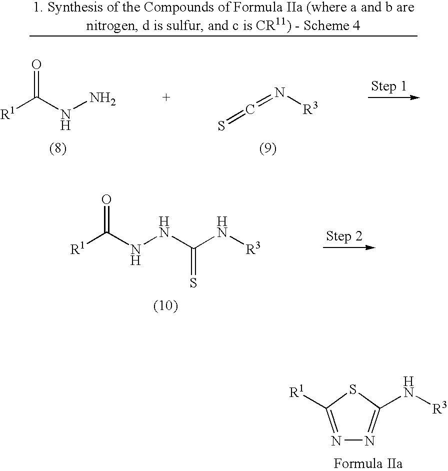

The thiadiazole derivative, represented by formula Ia, can be prepared as shown in Scheme 4.

Step-1 Preparation of Formula (10)

Compound of formula (10) was prepared by reaction of a compound of formula (8) (e.g. m-toluic hydrazide) with an isothiocyanate of formula (9), in a basic solvent (e.g. pyridine), typically heated near 65° C. for 2-6 hours.

Step-2 Preparation of Formula IIa

Compound of formula IIa was prepared by dissolving a compound of formula (10) in POCl₃ and heated near 80° C. for 2-4 hours. When the reaction is substantially complete, the product of formula Ia is isolated by conventional means (e.g. reverse phase HPLC).

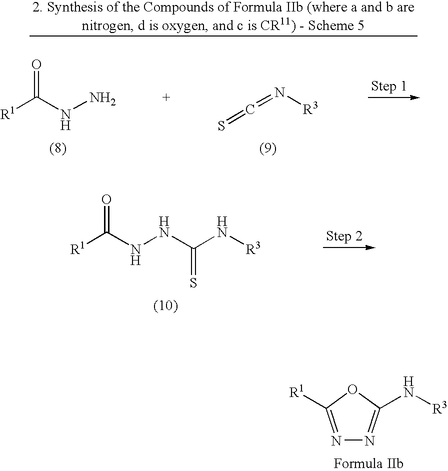

The oxadiazole derivatives, represented by formula IIb, can be prepared as shown in Scheme 5.

Step-1 Preparation of Formula (10)

Compound of formula (10) was prepared by reaction of a compound of formula (8), e.g. m-toluic hydrazide, with an isothiocyanate of formula (9), in a basic solvent (e.g. pyridine), typically heated near 65° C. for 2-6 hours.

Step-2 Preparation of Formula IIb

Compound of formula IIb was prepared by dissolving a compound of formula (10) in SOCl₂ and heated near 80° C. for 2-4 hours. When the reaction is substantially complete, the product of formula II was isolated by conventional means (e.g. reverse phase HPLC).

### Example 11

### Synthesis of the Compounds of Formula IIIa, where n=0

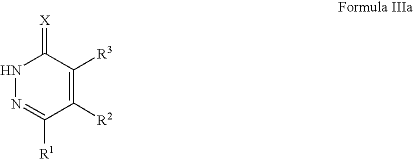 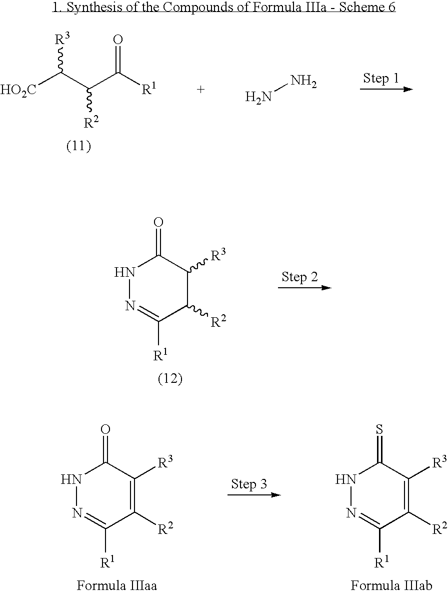 where the squiggly lines indicate a mixture of stereoisomers.

The Pyridazinone derivatives, represented by formula IIIa, can be prepared as shown in Scheme 6.

Step-1 Preparation of Formula (12)

The compound of formula (12) was prepared by reaction of a 4-oxo-butyric acid derivative of formula (11) with hydrazine, in a basic solvent (e.g. pyridine), typically heated near 65° C. for 2-6 hours.

Step-2 Preparation of Formula IIIaa

The compound of formula IIIaa was prepared by dissolving a compound of formula (12) in an inert solvent and adding an oxidizing agent (e.g. Br₂, chloranil, or Pd(C) under an air atmosphere. When the reaction is substantially complete, the product of formula IIIaa is isolated by conventional means (e.g. reverse phase HPLC).

Step-3 Preparation of Formula IIIab

Compounds of Formula IIIaa (X=0) can be converted into compounds where X=S, by treatment with Lawesson's reagent or P₂S₅ stirred in an inert solvent at ambient temperature for 2-6 hours. When the reaction is substantially complete, the product of formula IIIb is isolated by conventional means (e.g. reverse phase HPLC).

Synthesis of the compounds of Formula IIIb, where n=1:

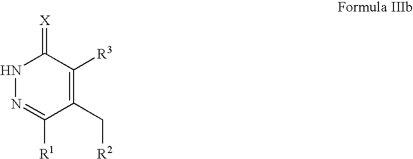

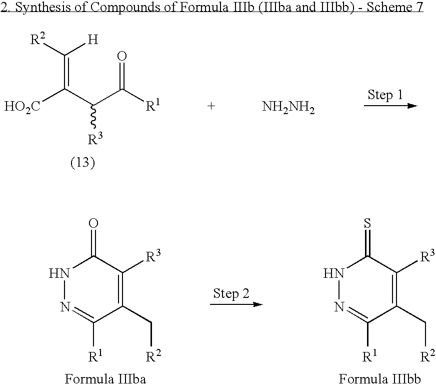

The Pyridazinone derivatives, represented by formula IIIba, can be prepared as shown in Scheme 7.

Step-1 Preparation of Formula IIIba

The compound of formula IIIba was prepared by reaction of an acyl-acrylic acid of formula (13) with hydrazine, in a basic solvent (e.g. aqueous NaOH), typically stirred at ambient temperature for 2-6 hours. When the reaction is substantially complete, the product of formula IIIa (X=0) is isolated by conventional means (e.g. reverse phase HPLC).

Step-2 Preparation of Formula IIIbb

Compounds of Formula IIIba (X=0) can be converted into compounds where X S, by treatment with Lawesson's reagent or P₂S₅ stirred in an inert solvent at ambient temperature for 2-6 hours. When the reaction is substantially complete, the product of formula IIIbb is isolated by conventional means (e.g. reverse phase HPLC).

### Example 12

### Synthesis of the Compounds of Formula IV

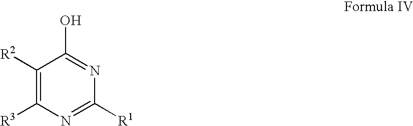

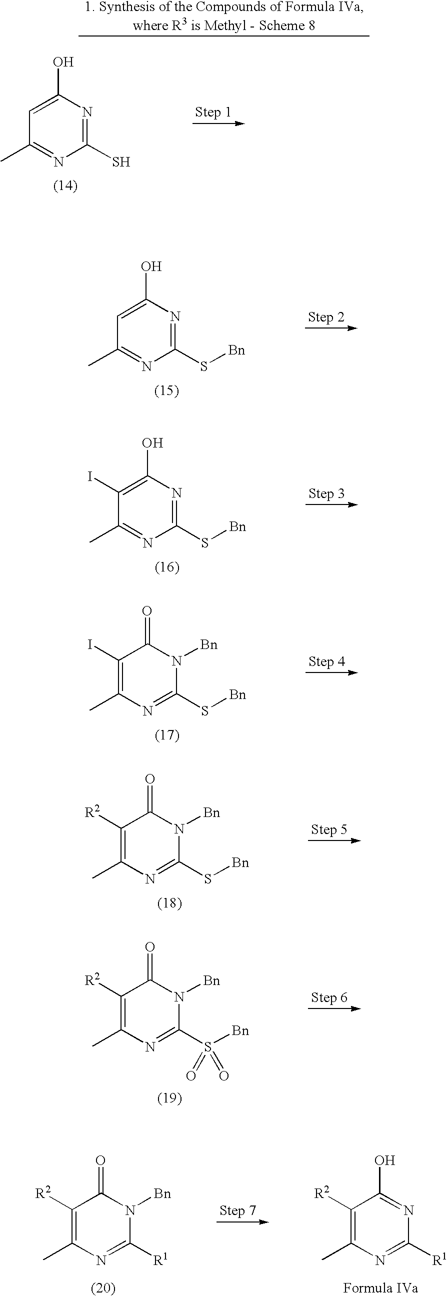

The 4-hydroxypyrimidine derivatives, represented by formula IVa, can be prepared as shown in Scheme 8.

Step-1 Preparation of Formula (15)

Compound (15) was prepared by reaction of compound (14) with benzyl bromide in an inert solvent (e.g. DMF), in the presence of a base (e.g. Et₃N), at room temperature for 4 hours. Product (15) is purified by a conventional way (e.g. recrystallization).

Step-2 Preparation of Formula (16)

Compound (16) was prepared by reaction of compound (15) with N-iodosuccinimide in chloroform under reflux for several hours. Product (16) was purified by a conventional way (e.g. recrystallization).

Step-3 Preparation of Formula (17)

Compound (17) was prepared by reaction of compound (16) with benzyl bromide in an inert solvent (e.g. DMF), in the presence of a base (e.g. NaH) at room temperature for several hours.

Step 4 Preparation of Formula (18)

The compound of formula (18) was prepared by reaction of compound (17), with a suitable reagent for coupling reaction (e.g. phenylboronic acid) in a suitable mixture of solvent (e.g. dimethoxyethane and water), in the presence of a base (e.g. K₂CO₃), typically heated to 100° C. for several hours. The product was isolated by a conventional way (e.g. flash chromatography).

Step 5 Preparation of Formula (19)

The compound of formula (19) was prepared by reaction of a compound of formula (18), with an oxidizing reagent (e.g. MCPBA) in a suitable solvent (e.g. CH₂Cl₂), typically at room temperature for a few hours.

Step 6 Preparation of Formula (20)

The compound of formula (20) was prepared by reaction of a compound of formula (19) with a nucleophilic reagent (e.g. piperazine), in the presence of a base (e.g. Cs₂CO₃) in a suitable solvent (e.g. dioxane) reflux for several hours. The product is purified in a conventional way (e.g. flash chromatography).

Step 7 Preparation of Formula IV

The compound of formula IVa was prepared conventionally by hydrogenation of a compound of formula (20) in the presence of a catalyst (e.g. Pd(OH)₂/C), under hydrogen in a suitable solvent (e.g. methanol) at room temperature for several hours.

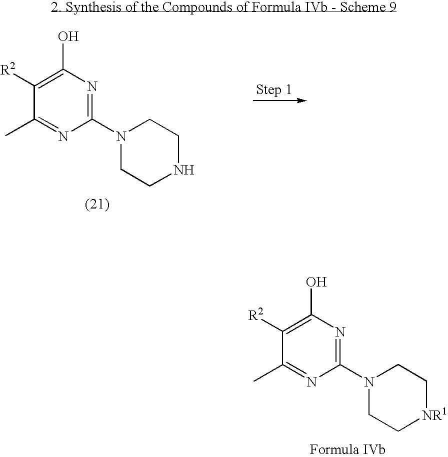

The 4-hydroxypyrimidine derivatives, represented by formula IV, can be prepared as shown in Scheme 9.

Step 1 Preparation of Formula IVb

The compound of formula IVb was prepared by reaction of a compound of formula (21) with an electrophilic reagent (phenyl isocyanate), in the presence of a base (e.g. Et₃N) in a suitable solvent (e.g. CH₂Cl₂) at room temperature for several hours. When the reaction is substantially complete, the product of formula IVb was isolated by conventional means (e.g. flash chromatography).

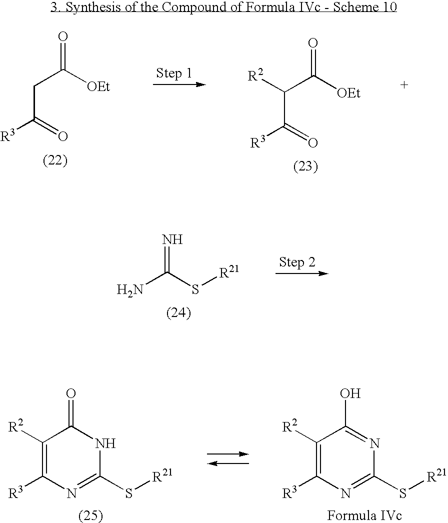

The 4-hydroxypyrimidine derivatives, represented by formula IVc, were prepared as shown in Scheme 10.

Step-1 Preparation of Formula (23)

The compound of formula (23) was prepared by reaction of a compound of formula (22), with an alkyl halide reagent (e.g. methyl iodide) in an inert solvent (e.g. DMF), in the presence of a base (e.g. K₂CO₃), typically heated near 80° C. for 12-36 hours.

Step-2 Preparation of Formula IVc

The compound of formula (25) was prepared y by reaction of a compound of formula (23) with an alkylisothiourea (e.g. S-methylisothiourea, H₂NCNHSCH₃), while heating in a suitable solvent (e.g. ethanol) at 75° C. for several hours. When the reaction is substantially complete, the product of formula IVc was isolated by conventional means; for example, recrystallization.

4. Alternate Synthesis of the Compounds of Formula IV—Scheme 11

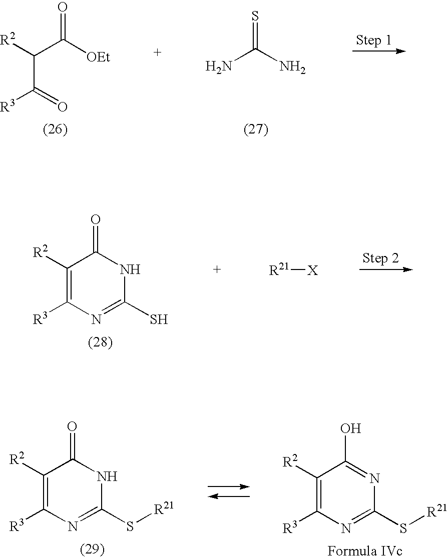

Step-1 Preparation of Formula (28)

Compound of formula (28) was prepared conventionally by reaction of a compound of formula (26), with thiourea of formula (27), in a polar solvent (e.g. ethanol), and typically heated near 80° C. for 12-36 hours and isolating the product by column chromatography.

Step-2 Preparation of Formula (29)

Compound of formula (29) was prepared by reaction of a compound of formula (28) with an alkylating agent (e.g. ethylbromoacetate), in the presence of a base (e.g. K₂CO₃) in a suitable solvent (e.g. acetonitrile) at room temperature or reflux for several hours. When the reaction is substantially complete, the product of formula IVc was isolated by conventional means.

### Example 13

### Synthesis of the Compounds of Formula V

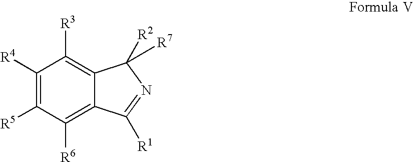

1. Synthesis of the Compounds of Formula Va, where R¹, R², R³, and R⁶ are Methyl; R⁴ and R⁵ are Hydrogen—Scheme 12

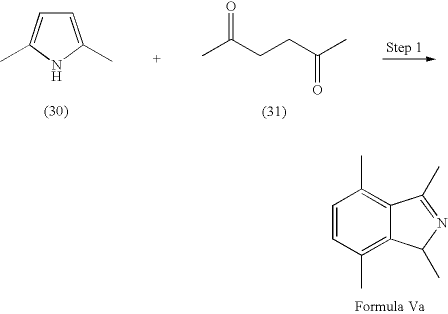

The isoindole derivatives, represented by formula Va, can be prepared as shown in Scheme 12.

Step-1 Preparation of Formula Va

The compound of formula (30) can be reacted with diketone compounds of formula (31), in aqueous acetic acid and typically heated near 100° C. for 12-36 hours. The compound can be isolated by conventional methods (e.g. recrystallization).

2. Synthesis of the Compounds of Formula Vb, where R³, R⁶, R⁴ and R⁵ are Hydrogen; R⁷ is hydroxy—Scheme 13

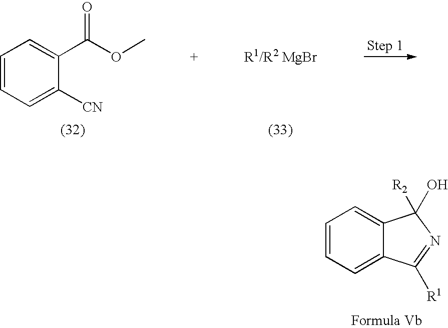

Step-1 Preparation of Formula Vb

The compound of Formula Vb is prepared conventionally by reaction of a compound of formula (32) with a Grignard reagent (e.g. Phenyl magnesium bromide), in a suitable solvent (e.g. benzene) and refluxed for 1 hour. When the reaction is substantially complete, the product of is isolated by conventional means (e.g., column chromatography).

3. Synthesis of the Compounds of Formula Vc, where where R¹, R², R³, R⁴, R⁵, R⁶, and R⁷ are Hydrogen—Scheme 14

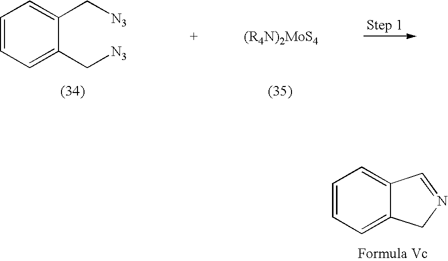

Step-1 Preparation of Vc

The compound (34) and the ammonium tetrathiomolybdate of formula (35) are reacted at room temperature with aqueous CH₃CN as the solvent. When the reaction is substantially complete (typically 1 hour), the solvent is removes by evaporation at reduced pressure. The product Vc is isolated by conventional means (e.g. ether extraction). (Ramesha, et. al., J. Org. Chem., 1995, 60, 7682-7683; and references therein).

### Example 14

### Synthesis of the Compounds of Formula VI

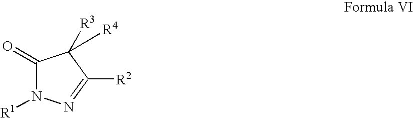

1. Synthesis of the Compounds of Formula VIa, where R⁴=H—Scheme 15

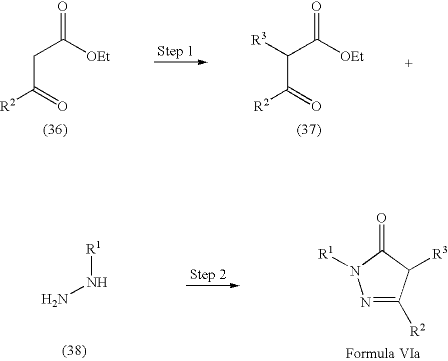

Pyrazolidinone derivatives, represented by formula VI, can be prepared as shown in Scheme-15.

Step-1 Preparation of Formula (37)

The compound of formula (37) is prepared conventionally by reaction of a compound of formula (36), with an alkyl halide reagent (e.g. methyl iodide) in an inert solvent (e.g. DMF), in the presence of a base (e.g. K₂CO₃), typically heated near 80° C. for 12-36 hours.

Step-2 Preparation of Formula VIa

Compound of formula (37) is reacted with hydrazine of formula (38) along with a catalytic amount of concentrated acid (e.g. hydrochloric acid) at 120° C. The liberation of alcohol and water results in the formation of the product of formula VIa, can be isolated through conventional means, for example column chromatography.

2. Alternate Synthesis of the Compounds of Formula VI, where R⁴═H:—Scheme 16

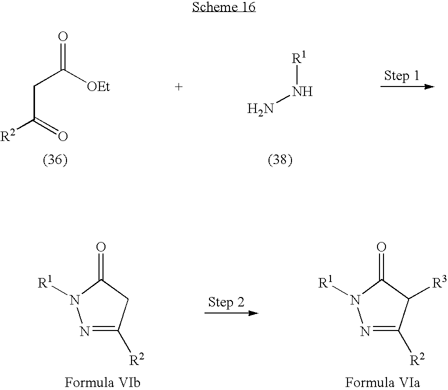

Step-1 Preparation of Formula VIb

Compound of formula (36) is reacted with hydrazine of formula (38) along with a catalytic amount of concentrated acid (e.g. hydrochloric acid) at 120° C. The liberation of alcohol and water results in the formation of the product of formula VIb, can be isolated through conventional means, for example column chromatography.

Step-2 Preparation of Formula VIa:

The compound of formula VI is prepared conventionally by reaction of a compound of formula (42) with an alkylating agent of formula (43) (e.g. ethylbromoacetate), in the presence of a base (e.g. K₂CO₃) in a suitable solvent (e.g. acetonitrile) at room temperature or reflux for several hours. When the reaction is substantially complete, the product of Formula VI is isolated by conventional means; for example, recrystallization.

### Example 15

### Synthesis of the Compounds of Formula VII

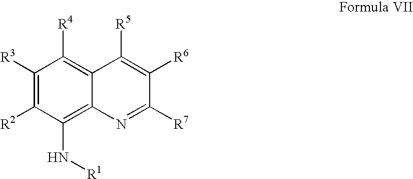

1. Synthesis of the Compounds of Formula VII—Scheme 17

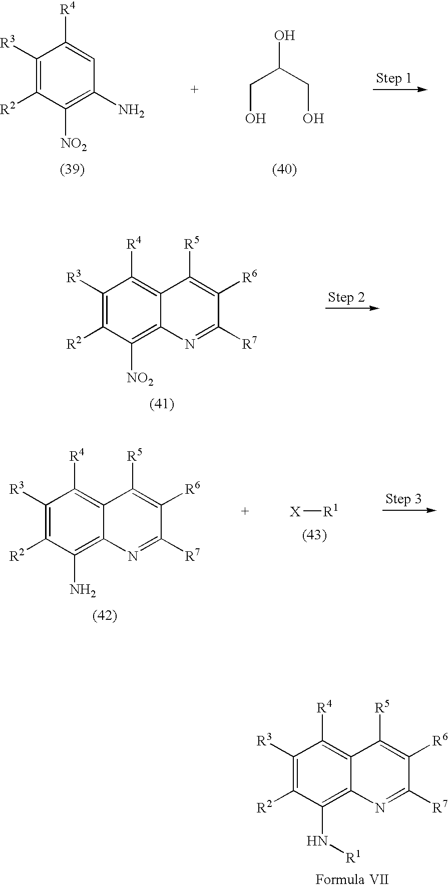

The 8-aminoquinoline derivatives, represented by formula VII, can be prepared as shown in Scheme-17. In this particular scheme, R⁵, R⁶, and R⁷ are hydrogen.

Step-1 Preparation of Formula (41)

The compound of formula (41) can be prepared via a Skraup reaction by reaction of a compound of formula (39) (e.g. 2-nitroaniline), with glycerol (40), in the presence of sulfuric acid in an inert solvent (e.g. dioxane) and typically heated to >100° C. for 2-24 hours. The addition of nitrobenzene or arsenic oxide can aid the reaction (Claus and Schoeller, J. Prakt. Chem. 1893, 48, 140. Mosher, H. S. et. al., Org. Syn. CV 3, 568).

Step-2 Preparation of Formula (42)

The compound of formula (42) can be prepared conventionally by reaction of a compound of formula (41) with a reducing agent (e.g. hydrogen gas, ammonium formate, HCO₂NH₄), in the presence of a catalyst (e.g. Pd/C), in a suitable solvent (e.g. methanol) at room temperature for several hours. When the reaction is substantially complete, the product of formula (41) can be isolated by conventional means, (e.g. filtration through Celite).

Step-3 Preparation of Formula VII

The compound of formula (42) can be reacted with a compound of formula (43) where X is a leaving group (e.g. bromide, chloride) or an electrophilic substituent (e.g. isocyanate, isothiocyanate), in the presence of base (e.g. K₂CO₃), in an inert solvent (e.g. DMF). Representaive examples of compounds of formula (43) include benzoyl chloride, benzenesulfonyl chloride, 3-bromo-2-methylpropane, benzyl bromide, phenyl isocyanate, and phenyl isothiocyanate. When the reaction is substantially complete, the product of formula VII can be isolated by conventional means (e.g. silica gel chromatography).

2. Alternative Exemplary Synthesis of Compounds of Formula VII—Scheme 18

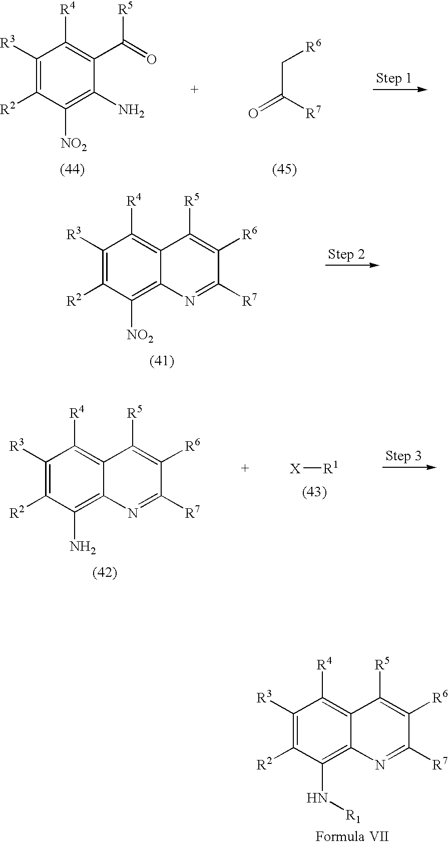

The 8-aminoquinoline derivatives, represented by formula VII, can be prepared as shown in Scheme-18.

Step-1 Preparation of Formula (41)

The compound of formula (41) can be prepared via a Friedländer synthesis by reaction of a compound of formula (44) (e.g. 2-amino-3-nitro-acetophenone), with a compound of formula (45) (e.g. acetone), in the presence of base (e.g. potassium hydroxide, piperidine) in an inert solvent (e.g. ethanol) and possible with heating for 2-24 hours (Eckert, K. Angew. Chem. Int. Ed. 1981, 20, 208).

Step-2 Preparation of Formula (42)

The compound of formula (42) can be prepared conventionally by reaction of a compound of formula (41) with a reducing agent (e.g. hydrogen gas, ammonium formate, HCO₂NH₄), in the presence of a catalyst (e.g. Pd/C), in a suitable solvent (e.g. methanol) at room temperature for several hours. When the reaction is substantially complete, the product of formula (42) can be isolated by conventional means, (e.g. filtration through Celite).

Step-3 Preparation of Formula VII

The compound of formula (42) can be reacted with a compound of formula (48) where X is a leaving group (e.g. bromide, chloride) or an electrophilic substituent (e.g. isocyanate, isothiocyanate), in the presence of base (e.g. K₂CO₃), in an inert solvent (e.g. DMF). Representaive examples of compounds of formula (43) include benzoyl chloride, benzenesulfonyl chloride, 3-bromo-2-methylpropane, benzyl bromide, phenyl isocyanate, and phenyl isothiocyanate. When the reaction is substantially complete, the product of formula VII can be isolated by conventional means (e.g. silica gel chromatography).

### Example 16

### Site-Directed Mutagenesis of Kinases

Mutagenesis of kinases, e.g. PIM kinases, such as the P123M mutation of PIM-1 can be carried out according to the following procedure (or other procedures available persons performing molecular biological techniques) as described in Molecular Biology: Current Innovations and Future Trends. Eds. A. M. Griffin and H. G. Griffin. (1995) ISBN 1-898486-01-8, Horizon Scientific Press, PO Box 1, Wymondham, Norfolk, U.K., among others.

In vitro site-directed mutagenesis is an invaluable technique for studying protein structure-function relationships, gene expression and vector modification. Several methods have appeared in the literature, but many of these methods require single-stranded DNA as the template. The reason for this, historically, has been the need for separating the complementary strands to prevent reannealing. Use of PCR in site-directed mutagenesis accomplishes strand separation by using a denaturing step to separate the complementing strands and allowing efficient polymerization of the PCR primers. PCR site-directed methods thus allow site-specific mutations to be incorporated in virtually any double-stranded plasmid; eliminating the need for M13-based vectors or single-stranded rescue.

It is often desirable to reduce the number of cycles during PCR when performing PCR-based site-directed mutagenesis to prevent clonal expansion of any (undesired) second-site mutations. Limited cycling which would result in reduced product yield, is offset by increasing the starting template concentration. A selection is used to reduce the number of parental molecules coming through the reaction. Also, in order to use a single PCR primer set, it is desirable to optimize the long PCR method. Further, because of the extendase activity of some thermostable polymerases it is often necessary to incorporate an end-polishing step into the procedure prior to end-to-end ligation of the PCR-generated product containing the incorporated mutations in one or both PCR primers.

The following protocol provids as a facile method for site-directed mutagenesis and accomplishes the above desired features by the incorporation of the following steps:
(i) increasing template concentration approximately 1000-fold over conventional PCR conditions; (ii) reducing the number of cycles from 25-30 to 5-10; (iii) adding the restriction endonuclease DpnI (recognition target sequence: 5-Gm6ATC-3, where the A residue is methylated) to select against parental DNA (note: DNA isolated from almost all common strains of E. coli is Dam-methylated at the sequence 5-GATC-3); (iv) using Taq Extender in the PCR mix for increased reliability for PCR to 10 kb; (v) using Pfu DNA polymerase to polish the ends of the PCR product, and (vi) efficient intramolecular ligation in the presence of T4 DNA ligase.

Plasmid template DNA (approximately 0.5 pmole) is added to a PCR cocktail containing, in 25 ul of 1× mutagenesis buffer: (20 mM Tris HCl, pH 7.5; 8 mM MgCl2; 40 ug/ml BSA); 12-20 pmole of each primer (one of which must contain a 5-prime phosphate), 250 uM each dNTP, 2.5 U Taq DNA polymerase, 2.5 U of Taq Extender (Stratagene).

The PCR cycling parameters are 1 cycle of: 4 min at 94 C, 2 min at 50 C and 2 min at 72 C; followed by 5-10 cycles of 1 min at 94 C, 2 min at 54 C and 1 min at 72 C (step 1).

The parental template DNA and the linear, mutagenesis-primer incorporating newly synthesized DNA are treated with DpnI (10 U) and Pfu DNA polymerase (2.5U). This results in the DpnI digestion of the in vivo methylated parental template and hybrid DNA and the removal, by Pfu DNA polymerase, of the Taq DNA polymerase-extended base(s) on the linear PCR product.

The reaction is incubated at 37 C for 30 min and then transferred to 72 C for an additional 30 min (step 2).

Mutagenesis buffer (lx, 115 ul, containing 0.5 mM ATP) is added to the DpnI-digested, Pfu DNA polymerase-polished PCR products.

The solution is mixed and 10 ul is removed to a new microfuge tube and T4 DNA ligase (2-4 U) added.

The ligation is incubated for greater than 60 min at 37 C (step 3).

The treated solution is transformed into competent E. coli (step 4).

In addition to the PCT-based site-directed mutagenesis described above, other methods are available. Examples include those described in Kunkel (1985) Proc. Natl. Acad. Sci. 82: 488-492; Eckstein et al. (1985) Nucl. Acids Res. 13: 8764-8785; and using the GeneEditor™ Site-Directed Mutageneis Sytem from Promega.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to crystallization or co-crystallization conditions for PIM proteins. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

TABLE 1

| HEADER | | ---- XX-XXX-XX xxxx | | | |
|---|---|---|---|---|---|
| COMPND | | --- | | | |
| REMARK | 3 | | | | |
| REMARK | 3 | REFINEMENT. | | | |
| REMARK | 3 | PROGRAM: REFMAC 5.1.19 | | | |
| REMARK | 3 | AUTHORS: MURSHUDOV, VAGIN, DODSON | | | |
| REMARK | 3 | | | | |
| REMARK | 3 | REFINEMENT TARGET: MAXIMUM LIKELIHOOD | | | |
| REMARK | 3 | | | | |
| REMARK | 3 | DATA USED IN REFINEMENT. | | | |
| REMARK | 3 | RESOLUTION RANGE HIGH (ANGSTROMS): 2.00 | | | |
| REMARK | 3 | RESOLUTION RANGE LOW (ANGSTROMS): 84.52 | | | |
| REMARK | 3 | DATA CUTOFF (SIGMA(F)): NONE | | | |
| REMARK | 3 | COMPLETENESS FOR RANGE (%): 99.27 | | | |
| REMARK | 3 | NUMBER OF REFLECTIONS: 28693 | | | |
| REMARK | 3 | | | | |
| REMARK | 3 | FIT TO DATA USED IN REFINEMENT. | | | |
| REMARK | 3 | CROSS-VALIDATION METHOD: THROUGHOUT | | | |
| REMARK | 3 | FREE R VALUE TEST SET SELECTION: RANDOM | | | |
| REMARK | 3 | R VALUE (WORKING + TEST SET): 0.22119 | | | |
| REMARK | 3 | R VALUE (WORKING SET): 0.22012 | | | |
| REMARK | 3 | FREE R VALUE: 0.24194 | | | |
| REMARK | 3 | FREE R VALUE TEST SET SIZE (%): 5.0 | | | |
| REMARK | 3 | FREE R VALUE TEST SET COUNT: 1498 | | | |
| REMARK | 3 | | | | |
| REMARK | 3 | FIT IN THE HIGHEST RESOLUTION BIN. | | | |
| REMARK | 3 | TOTAL NUMBER OF BINS USED: 20 | | | |
| REMARK | 3 | BIN RESOLUTION RANGE HIGH: 2.000 | | | |
| REMARK | 3 | BIN RESOLUTION RANGE LOW: 2.052 | | | |
| REMARK | 3 | REFLECTION IN BIN (WORKING SET): 2096 | | | |
| REMARK | 3 | BIN R VALUE (WORKING SET): 0.344 | | | |
| REMARK | 3 | BIN FREE R VALUE SET COUNT: 102 | | | |
| REMARK | 3 | BIN FREE R VALUE: 0.359 | | | |
| REMARK | 3 | | | | |
| REMARK | 3 | NUMBER OF NON-HYDROGEN ATOMS USED IN REFINEMENT. | | | |
| REMARK | 3 | ALL ATOMS: 2382 | | | |
| REMARK | 3 | | | | |
| REMARK | 3 | B VALUES. | | | |
| REMARK | 3 | FROM WILSON PLOT (A**2): NULL | | | |
| REMARK | 3 | MEAN B VALUE (OVERALL, A**2): 49.236 | | | |
| REMARK | 3 | OVERALL ANISOTROPIC B VALUE. | | | |
| REMARK | 3 | B11 (A**2): 1.32 | | | |
| REMARK | 3 | B22 (A**2): 1.32 | | | |
| REMARK | 3 | B33 (A**2): −1.99 | | | |
| REMARK | 3 | B12 (A**2): 0.66 | | | |
| REMARK | 3 | B13 (A**2): 0.00 | | | |
| REMARK | 3 | B23 (A**2): 0.00 | | | |
| REMARK | 3 | | | | |
| REMARK | 3 | ESTIMATED OVERALL COORDINATE ERROR. | | | |
| REMARK | 3 | ESU BASED ON R VALUE (A): 0.158 | | | |
| REMARK | 3 | ESU BASED ON FREE R VALUE (A): 0.142 | | | |
| REMARK | 3 | ESU BASED ON MAXIMUM LIKELIHOOD (A): 0.127 | | | |
| REMARK | 3 | ESU FOR B VALUES BASED ON MAXIMUM LIKELIHOOD (A**2): 4.758 | | | |
| REMARK | 3 | | | | |
| REMARK | 3 | CORRELATION COEFFICIENTS. | | | |
| REMARK | 3 | CORRELATION COEFFICIENT FO-FC: 0.954 | | | |
| REMARK | 3 | CORRELATION COEFFICIENT FO-FC FREE: 0.947 | | | |
| REMARK | 3 | | | | |
| REMARK | 3 | RMS DEVIATIONS FROM IDEAL VALUES | COUNT | RMS | WEIGHT |
| REMARK | 3 | BOND LENGTHS REFINED ATOMS (A): | 2296; | 0.011; | 0.021 |
| REMARK | 3 | BOND ANGLES REFINED ATOMS (DEGREES): | 3114; | 1.088; | 1.945 |
| REMARK | 3 | TORSION ANGLES, PERIOD 1 (DEGREES): | 273; | 3.838; | 5.000 |
| REMARK | 3 | CHIRAL-CENTER RESTRAINTS (A**3): | 332; | 0.081; | 0.200 |
| REMARK | 3 | GENERAL PLANES REFINED ATOMS (A): | 1784; | 0.004; | 0.020 |
| REMARK | 3 | NON-BONDED CONTACTS REFINED ATOMS (A): | 1094; | 0.215; | 0.200 |
| REMARK | 3 | H-BOND (X...Y) REFINED ATOMS (A): | 138; | 0.121; | 0.200 |
| REMARK | 3 | SYMMETRY VDW REFINED ATOMS (A): | 60; | 0.282; | 0.200 |
| REMARK | 3 | SYMMETRY H-BOND REFINED ATOMS (A): | 19; | 0.247; | 0.200 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2): | 1365; | 1.058; | 1.500 |
| REMARK | 3 | MAIN-CHAIN ANGLE REFINED ATOMS (A**2): | 2212; | 2.010; | 2.000 |
| REMARK | 3 | SIDE-CHAIN BOND REFINED ATOMS (A**2): | 931; | 2.240; | 3.000 |
| REMARK | 3 | SIDE-CHAIN ANGLE REFINED ATOMS (A**2): | 902; | 3.766; | 4.500 |

| | | |
|---|---|---|
| REMARK | 3 | |
| REMARK | 3 | NCS RESTRAINTS STATISTICS |
| REMARK | 3 | NUMBER OF NCS GROUPS: NULL |
| REMARK | 3 | |
| REMARK | 3 | |
| REMARK | 3 | TLS DETAILS |
| REMARK | 3 | NUMBER OF TLS GROUPS: NULL |
| REMARK | 3 | |
| REMARK | 3 | |
| REMARK | 3 | BULK SOLVENT MODELLING. |
| REMARK | 3 | METHOD USED: BABINET MODEL WITH MASK |
| REMARK | 3 | PARAMETERS FOR MASK CALCULATION |
| REMARK | 3 | VDW PROBE RADIUS: 1.40 |
| REMARK | 3 | ION PROBE RADIUS: 0.80 |
| REMARK | 3 | SHRINKAGE RADIUS: 0.80 |
| REMARK | 3 | |
| REMARK | 3 | OTHER REFINEMENT REMARKS: NULL |
| REMARK | 3 | |
| CISPEP | 1 | GLU A 124 PRO A 125 0.00 |
| CRYST1 | | 99.210 99.210 80.285 90.00 90.00 120.00 P 65 |
| SCALE1 | | 0.010080 0.005819 0.000000 0.00000 |
| SCALE2 | | 0.000000 0.011639 0.000000 0.00000 |
| SCALE3 | | 0.000000 0.000000 0.012456 0.00000 |

| | | | | | | | | | | | |
|---|---|---|---|---|---|---|---|---|---|---|---|
| ATOM | 1 | N | PRO | A | 33 | 9.285 | 100.137 | −4.493 | 1.00 | 93.84 | N |
| ATOM | 2 | CA | PRO | A | 33 | 8.922 | 99.154 | −3.430 | 1.00 | 93.59 | C |
| ATOM | 3 | CB | PRO | A | 33 | 9.624 | 97.864 | −3.896 | 1.00 | 93.79 | C |
| ATOM | 4 | CG | PRO | A | 33 | 10.732 | 98.328 | −4.833 | 1.00 | 93.76 | C |
| ATOM | 5 | CD | PRO | A | 33 | 10.201 | 99.562 | −5.499 | 1.00 | 93.83 | C |
| ATOM | 6 | C | PRO | A | 33 | 9.413 | 99.588 | −2.038 | 1.00 | 93.22 | C |
| ATOM | 7 | O | PRO | A | 33 | 8.647 | 100.212 | −1.288 | 1.00 | 93.33 | O |
| ATOM | 8 | N | LEU | A | 34 | 10.667 | 99.251 | −1.716 | 1.00 | 92.55 | N |
| ATOM | 9 | CA | LEU | A | 34 | 11.325 | 99.616 | −0.457 | 1.00 | 91.82 | C |
| ATOM | 10 | CB | LEU | A | 34 | 11.402 | 101.150 | −0.303 | 1.00 | 92.11 | C |
| ATOM | 11 | CG | LEU | A | 34 | 12.362 | 101.709 | 0.756 | 1.00 | 92.47 | C |
| ATOM | 12 | CD1 | LEU | A | 34 | 13.829 | 101.513 | 0.349 | 1.00 | 92.34 | C |
| ATOM | 13 | CD2 | LEU | A | 34 | 12.044 | 103.183 | 1.024 | 1.00 | 93.01 | C |
| ATOM | 14 | C | LEU | A | 34 | 10.758 | 98.941 | 0.808 | 1.00 | 90.98 | C |
| ATOM | 15 | O | LEU | A | 34 | 11.164 | 97.828 | 1.157 | 1.00 | 91.10 | O |
| ATOM | 16 | N | GLU | A | 35 | 9.837 | 99.614 | 1.498 | 1.00 | 89.80 | N |
| ATOM | 17 | CA | GLU | A | 35 | 9.346 | 99.114 | 2.780 | 1.00 | 88.50 | C |
| ATOM | 18 | CB | GLU | A | 35 | 10.297 | 99.526 | 3.901 | 1.00 | 88.76 | C |
| ATOM | 19 | CG | GLU | A | 35 | 10.444 | 101.039 | 4.047 | 1.00 | 89.07 | C |
| ATOM | 20 | CD | GLU | A | 35 | 11.208 | 101.436 | 5.292 | 1.00 | 89.82 | C |
| ATOM | 21 | OE1 | GLU | A | 35 | 10.603 | 101.403 | 6.400 | 1.00 | 90.45 | O |
| ATOM | 22 | OE2 | GLU | A | 35 | 12.411 | 101.780 | 5.162 | 1.00 | 89.60 | O |
| ATOM | 23 | C | GLU | A | 35 | 7.963 | 99.672 | 3.060 | 1.00 | 87.48 | C |
| ATOM | 24 | O | GLU | A | 35 | 7.220 | 99.114 | 3.875 | 1.00 | 87.62 | O |
| ATOM | 25 | N | SER | A | 36 | 7.640 | 100.781 | 2.382 | 1.00 | 85.74 | N |
| ATOM | 26 | CA | SER | A | 36 | 6.316 | 101.427 | 2.424 | 1.00 | 83.76 | C |
| ATOM | 27 | CB | SER | A | 36 | 6.258 | 102.576 | 1.402 | 1.00 | 84.10 | C |
| ATOM | 28 | OG | SER | A | 36 | 7.465 | 103.332 | 1.399 | 1.00 | 84.47 | O |
| ATOM | 29 | C | SER | A | 36 | 5.170 | 100.444 | 2.150 | 1.00 | 81.91 | C |
| ATOM | 30 | O | SER | A | 36 | 3.997 | 100.755 | 2.389 | 1.00 | 81.51 | O |
| ATOM | 31 | N | GLN | A | 37 | 5.535 | 99.262 | 1.651 | 1.00 | 79.60 | N |
| ATOM | 32 | CA | GLN | A | 37 | 4.600 | 98.179 | 1.363 | 1.00 | 77.25 | C |
| ATOM | 33 | CB | GLN | A | 37 | 5.316 | 97.058 | 0.614 | 1.00 | 77.48 | C |
| ATOM | 34 | CG | GLN | A | 37 | 6.195 | 97.509 | −0.554 | 1.00 | 77.20 | C |
| ATOM | 35 | CD | GLN | A | 37 | 6.645 | 96.330 | −1.414 | 1.00 | 77.20 | C |
| ATOM | 36 | OE1 | GLN | A | 37 | 5.827 | 95.483 | −1.799 | 1.00 | 77.03 | O |
| ATOM | 37 | NE2 | GLN | A | 37 | 7.942 | 96.268 | −1.709 | 1.00 | 76.81 | N |
| ATOM | 38 | C | GLN | A | 37 | 3.970 | 97.604 | 2.623 | 1.00 | 75.49 | C |
| ATOM | 39 | O | GLN | A | 37 | 2.879 | 97.043 | 2.567 | 1.00 | 75.51 | O |
| ATOM | 40 | N | TYR | A | 38 | 4.655 | 97.747 | 3.756 | 1.00 | 73.43 | N |
| ATOM | 41 | CA | TYR | A | 38 | 4.208 | 97.129 | 5.004 | 1.00 | 71.44 | C |
| ATOM | 42 | CB | TYR | A | 38 | 5.100 | 95.931 | 5.373 | 1.00 | 70.49 | C |
| ATOM | 43 | CG | TYR | A | 38 | 5.227 | 94.919 | 4.255 | 1.00 | 67.67 | C |
| ATOM | 44 | CD1 | TYR | A | 38 | 4.258 | 93.929 | 4.067 | 1.00 | 65.14 | C |
| ATOM | 45 | CE1 | TYR | A | 38 | 4.361 | 93.019 | 3.032 | 1.00 | 63.31 | C |
| ATOM | 46 | CZ | TYR | A | 38 | 5.446 | 93.087 | 2.177 | 1.00 | 62.94 | C |
| ATOM | 47 | OH | TYR | A | 38 | 5.568 | 92.191 | 1.151 | 1.00 | 64.24 | O |
| ATOM | 48 | CE2 | TYR | A | 38 | 6.417 | 94.054 | 2.339 | 1.00 | 63.82 | C |
| ATOM | 49 | CD2 | TYR | A | 38 | 6.304 | 94.967 | 3.371 | 1.00 | 65.13 | C |
| ATOM | 50 | C | TYR | A | 38 | 4.125 | 98.099 | 6.169 | 1.00 | 71.00 | C |
| ATOM | 51 | O | TYR | A | 38 | 5.021 | 98.914 | 6.385 | 1.00 | 70.68 | O |
| ATOM | 52 | N | GLN | A | 39 | 3.026 | 97.986 | 6.913 | 1.00 | 70.43 | N |
| ATOM | 53 | CA | GLN | A | 39 | 2.797 | 98.756 | 8.124 | 1.00 | 69.86 | C |
| ATOM | 54 | CB | GLN | A | 39 | 1.298 | 99.021 | 8.279 | 1.00 | 70.46 | C |
| ATOM | 55 | CG | GLN | A | 39 | 0.934 | 100.007 | 9.385 | 1.00 | 73.80 | C |
| ATOM | 56 | CD | GLN | A | 39 | 0.378 | 99.319 | 10.635 | 1.00 | 77.97 | C |
| ATOM | 57 | OE1 | GLN | A | 39 | −0.750 | 98.794 | 10.625 | 1.00 | 79.52 | O |
| ATOM | 58 | NE2 | GLN | A | 39 | 1.161 | 99.330 | 11.717 | 1.00 | 78.94 | N |
| ATOM | 59 | C | GLN | A | 39 | 3.333 | 97.967 | 9.322 | 1.00 | 68.49 | C |
| ATOM | 60 | O | GLN | A | 39 | 2.704 | 97.003 | 9.777 | 1.00 | 68.58 | O |
| ATOM | 61 | N | VAL | A | 40 | 4.491 | 98.390 | 9.834 | 1.00 | 66.87 | N |
| ATOM | 62 | CA | VAL | A | 40 | 5.141 | 97.688 | 10.940 | 1.00 | 65.53 | C |
| ATOM | 63 | CB | VAL | A | 40 | 6.600 | 98.137 | 11.138 | 1.00 | 65.20 | C |
| ATOM | 64 | CG1 | VAL | A | 40 | 7.310 | 97.201 | 12.100 | 1.00 | 64.63 | C |
| ATOM | 65 | CG2 | VAL | A | 40 | 7.336 | 98.174 | 9.804 | 1.00 | 65.16 | C |
| ATOM | 66 | C | VAL | A | 40 | 4.376 | 97.837 | 12.255 | 1.00 | 64.96 | C |
| ATOM | 67 | O | VAL | A | 40 | 3.833 | 98.893 | 12.547 | 1.00 | 65.27 | O |
| ATOM | 68 | N | GLY | A | 41 | 4.339 | 96.766 | 13.042 | 1.00 | 64.02 | N |
| ATOM | 69 | CA | GLY | A | 41 | 3.640 | 96.764 | 14.310 | 1.00 | 62.22 | C |
| ATOM | 70 | C | GLY | A | 41 | 4.545 | 96.341 | 15.451 | 1.00 | 61.31 | C |
| ATOM | 71 | O | GLY | A | 41 | 5.747 | 96.572 | 15.406 | 1.00 | 60.92 | O |
| ATOM | 72 | N | PRO | A | 42 | 3.966 | 95.725 | 16.478 | 1.00 | 60.62 | N |
| ATOM | 73 | CA | PRO | A | 42 | 4.723 | 95.313 | 17.666 | 1.00 | 60.91 | C |
| ATOM | 74 | CB | PRO | A | 42 | 3.636 | 94.755 | 18.602 | 1.00 | 60.81 | C |
| ATOM | 75 | CG | PRO | A | 42 | 2.347 | 95.332 | 18.089 | 1.00 | 60.97 | C |
| ATOM | 76 | CD | PRO | A | 42 | 2.529 | 95.401 | 16.599 | 1.00 | 60.64 | C |
| ATOM | 77 | C | PRO | A | 42 | 5.759 | 94.235 | 17.385 | 1.00 | 60.96 | C |
| ATOM | 78 | O | PRO | A | 42 | 5.626 | 93.478 | 16.424 | 1.00 | 60.93 | O |
| ATOM | 79 | N | LEU | A | 43 | 6.783 | 94.180 | 18.226 | 1.00 | 61.11 | N |
| ATOM | 80 | CA | LEU | A | 43 | 7.737 | 93.084 | 18.200 | 1.00 | 61.79 | C |
| ATOM | 81 | CB | LEU | A | 43 | 8.924 | 93.411 | 19.110 | 1.00 | 61.59 | C |
| ATOM | 82 | CG | LEU | A | 43 | 10.162 | 92.511 | 19.107 | 1.00 | 62.19 | C |
| ATOM | 83 | CD1 | LEU | A | 43 | 11.000 | 92.704 | 17.848 | 1.00 | 61.21 | C |
| ATOM | 84 | CD2 | LEU | A | 43 | 11.003 | 92.782 | 20.344 | 1.00 | 62.67 | C |
| ATOM | 85 | C | LEU | A | 43 | 7.027 | 91.795 | 18.643 | 1.00 | 62.48 | C |
| ATOM | 86 | O | LEU | A | 43 | 6.143 | 91.824 | 19.511 | 1.00 | 62.19 | O |
| ATOM | 87 | N | LEU | A | 44 | 7.396 | 90.671 | 18.030 | 1.00 | 63.26 | N |
| ATOM | 88 | CA | LEU | A | 44 | 6.811 | 89.378 | 18.387 | 1.00 | 63.89 | C |
| ATOM | 89 | CB | LEU | A | 44 | 6.257 | 88.663 | 17.154 | 1.00 | 63.70 | C |
| ATOM | 90 | CG | LEU | A | 44 | 5.135 | 89.362 | 16.379 | 1.00 | 63.05 | C |
| ATOM | 91 | CD1 | LEU | A | 44 | 4.801 | 88.562 | 15.131 | 1.00 | 62.30 | C |
| ATOM | 92 | CD2 | LEU | A | 44 | 3.894 | 89.539 | 17.241 | 1.00 | 62.27 | C |
| ATOM | 93 | C | LEU | A | 44 | 7.791 | 88.474 | 19.110 | 1.00 | 64.82 | C |
| ATOM | 94 | O | LEU | A | 44 | 7.386 | 87.669 | 19.951 | 1.00 | 65.08 | O |
| ATOM | 95 | N | GLY | A | 45 | 9.071 | 88.602 | 18.784 | 1.00 | 66.08 | N |
| ATOM | 96 | CA | GLY | A | 45 | 10.088 | 87.734 | 19.357 | 1.00 | 68.09 | C |
| ATOM | 97 | C | GLY | A | 45 | 11.517 | 88.122 | 19.027 | 1.00 | 69.52 | C |
| ATOM | 98 | O | GLY | A | 45 | 11.763 | 88.937 | 18.124 | 1.00 | 69.05 | O |
| ATOM | 99 | N | SER | A | 46 | 12.448 | 87.517 | 19.774 | 1.00 | 71.08 | N |
| ATOM | 100 | CA | SER | A | 46 | 13.891 | 87.764 | 19.662 | 1.00 | 72.58 | C |
| ATOM | 101 | CB | SER | A | 46 | 14.311 | 88.922 | 20.588 | 1.00 | 72.92 | C |
| ATOM | 102 | OG | SER | A | 46 | 15.655 | 89.327 | 20.364 | 1.00 | 74.04 | O |
| ATOM | 103 | C | SER | A | 46 | 14.688 | 86.513 | 20.027 | 1.00 | 73.06 | C |
| ATOM | 104 | O | SER | A | 46 | 14.265 | 85.720 | 20.875 | 1.00 | 73.26 | O |
| ATOM | 105 | N | GLY | A | 47 | 15.849 | 86.349 | 19.394 | 1.00 | 73.67 | N |
| ATOM | 106 | CA | GLY | A | 47 | 16.733 | 85.234 | 19.707 | 1.00 | 74.04 | C |
| ATOM | 107 | C | GLY | A | 47 | 17.739 | 84.965 | 18.608 | 1.00 | 74.12 | C |
| ATOM | 108 | O | GLY | A | 47 | 18.133 | 85.889 | 17.883 | 1.00 | 74.48 | O |
| ATOM | 109 | N | GLY | A | 48 | 18.150 | 83.698 | 18.490 | 1.00 | 73.84 | N |
| ATOM | 110 | CA | GLY | A | 48 | 19.109 | 83.257 | 17.478 | 1.00 | 73.16 | C |
| ATOM | 111 | C | GLY | A | 48 | 18.602 | 83.392 | 16.048 | 1.00 | 72.45 | C |
| ATOM | 112 | O | GLY | A | 48 | 19.391 | 83.374 | 15.093 | 1.00 | 72.37 | O |
| ATOM | 113 | N | PHE | A | 49 | 17.282 | 83.531 | 15.911 | 1.00 | 71.52 | N |
| ATOM | 114 | CA | PHE | A | 49 | 16.647 | 83.755 | 14.612 | 1.00 | 70.43 | C |
| ATOM | 115 | CB | PHE | A | 49 | 15.215 | 83.187 | 14.590 | 1.00 | 70.83 | C |
| ATOM | 116 | CG | PHE | A | 49 | 14.301 | 83.752 | 15.661 | 1.00 | 73.19 | C |
| ATOM | 117 | CD1 | PHE | A | 49 | 13.584 | 84.933 | 15.439 | 1.00 | 74.32 | C |
| ATOM | 118 | CE1 | PHE | A | 49 | 12.738 | 85.453 | 16.419 | 1.00 | 75.71 | C |
| ATOM | 119 | CZ | PHE | A | 49 | 12.587 | 84.787 | 17.638 | 1.00 | 75.96 | C |
| ATOM | 120 | CE2 | PHE | A | 49 | 13.290 | 83.605 | 17.874 | 1.00 | 75.42 | C |
| ATOM | 121 | CD2 | PHE | A | 49 | 14.139 | 83.090 | 16.883 | 1.00 | 74.82 | C |
| ATOM | 122 | C | PHE | A | 49 | 16.696 | 85.231 | 14.157 | 1.00 | 68.55 | C |
| ATOM | 123 | O | PHE | A | 49 | 16.785 | 85.509 | 12.963 | 1.00 | 69.15 | O |
| ATOM | 124 | N | GLY | A | 50 | 16.663 | 86.164 | 15.106 | 1.00 | 66.20 | N |
| ATOM | 125 | CA | GLY | A | 50 | 16.625 | 87.588 | 14.795 | 1.00 | 62.55 | C |
| ATOM | 126 | C | GLY | A | 50 | 15.562 | 88.351 | 15.578 | 1.00 | 59.75 | C |
| ATOM | 127 | O | GLY | A | 50 | 15.316 | 88.056 | 16.754 | 1.00 | 59.86 | O |
| ATOM | 128 | N | SER | A | 51 | 14.945 | 89.332 | 14.916 | 1.00 | 56.20 | N |
| ATOM | 129 | CA | SER | A | 51 | 13.866 | 90.148 | 15.480 | 1.00 | 51.75 | C |
| ATOM | 130 | CB | SER | A | 51 | 14.300 | 91.614 | 15.587 | 1.00 | 51.18 | C |
| ATOM | 131 | OG | SER | A | 51 | 15.454 | 91.750 | 16.401 | 1.00 | 48.30 | O |
| ATOM | 132 | C | SER | A | 51 | 12.699 | 90.076 | 14.537 | 1.00 | 49.79 | C |
| ATOM | 133 | O | SER | A | 51 | 12.848 | 90.341 | 13.344 | 1.00 | 48.22 | O |
| ATOM | 134 | N | VAL | A | 52 | 11.538 | 89.724 | 15.064 | 1.00 | 47.77 | N |
| ATOM | 135 | CA | VAL | A | 52 | 10.345 | 89.551 | 14.243 | 1.00 | 46.96 | C |
| ATOM | 136 | CB | VAL | A | 52 | 9.795 | 88.091 | 14.312 | 1.00 | 46.48 | C |
| ATOM | 137 | CG1 | VAL | A | 52 | 8.570 | 87.924 | 13.397 | 1.00 | 45.18 | C |
| ATOM | 138 | CG2 | VAL | A | 52 | 10.873 | 87.082 | 13.955 | 1.00 | 45.83 | C |
| ATOM | 139 | C | VAL | A | 52 | 9.265 | 90.515 | 14.701 | 1.00 | 47.44 | C |
| ATOM | 140 | O | VAL | A | 52 | 8.874 | 90.493 | 15.869 | 1.00 | 48.09 | O |
| ATOM | 141 | N | TYR | A | 53 | 8.784 | 91.346 | 13.779 | 1.00 | 47.68 | N |
| ATOM | 142 | CA | TYR | A | 53 | 7.723 | 92.315 | 14.055 | 1.00 | 48.21 | C |
| ATOM | 143 | CB | TYR | A | 53 | 8.102 | 93.711 | 13.532 | 1.00 | 47.13 | C |
| ATOM | 144 | CG | TYR | A | 53 | 9.290 | 94.335 | 14.223 | 1.00 | 46.28 | C |
| ATOM | 145 | CD1 | TYR | A | 53 | 10.593 | 93.949 | 13.897 | 1.00 | 43.98 | C |
| ATOM | 146 | CE1 | TYR | A | 53 | 11.689 | 94.495 | 14.532 | 1.00 | 42.59 | C |
| ATOM | 147 | CZ | TYR | A | 53 | 11.498 | 95.475 | 15.521 | 1.00 | 43.72 | C |
| ATOM | 148 | OH | TYR | A | 53 | 12.598 | 96.012 | 16.159 | 1.00 | 43.55 | O |
| ATOM | 149 | CE2 | TYR | A | 53 | 10.226 | 95.884 | 15.864 | 1.00 | 44.06 | C |
| ATOM | 150 | CD2 | TYR | A | 53 | 9.117 | 95.305 | 15.221 | 1.00 | 45.68 | C |
| ATOM | 151 | C | TYR | A | 53 | 6.436 | 91.886 | 13.363 | 1.00 | 49.25 | C |
| ATOM | 152 | O | TYR | A | 53 | 6.466 | 91.335 | 12.258 | 1.00 | 48.07 | O |
| ATOM | 153 | N | SER | A | 54 | 5.306 | 92.162 | 14.008 | 1.00 | 50.84 | N |
| ATOM | 154 | CA | SER | A | 54 | 3.996 | 91.959 | 13.398 | 1.00 | 53.09 | C |
| ATOM | 155 | CB | SER | A | 54 | 2.889 | 92.092 | 14.445 | 1.00 | 53.24 | C |
| ATOM | 156 | OG | SER | A | 54 | 1.609 | 91.939 | 13.854 | 1.00 | 55.73 | O |
| ATOM | 157 | C | SER | A | 54 | 3.826 | 93.019 | 12.342 | 1.00 | 54.41 | C |
| ATOM | 158 | O | SER | A | 54 | 4.303 | 94.129 | 12.510 | 1.00 | 55.46 | O |
| ATOM | 159 | N | GLY | A | 55 | 3.151 | 92.691 | 11.248 | 1.00 | 56.17 | N |
| ATOM | 160 | CA | GLY | A | 55 | 3.043 | 93.625 | 10.143 | 1.00 | 58.09 | C |
| ATOM | 161 | C | GLY | A | 55 | 1.800 | 93.391 | 9.327 | 1.00 | 60.22 | C |
| ATOM | 162 | O | GLY | A | 55 | 1.164 | 92.343 | 9.433 | 1.00 | 60.35 | O |
| ATOM | 163 | N | ILE | A | 56 | 1.457 | 94.381 | 8.513 | 1.00 | 62.12 | N |
| ATOM | 164 | CA | ILE | A | 56 | 0.307 | 94.305 | 7.635 | 1.00 | 64.34 | C |
| ATOM | 165 | CB | ILE | A | 56 | −0.847 | 95.188 | 8.169 | 1.00 | 64.42 | C |
| ATOM | 166 | CG1 | ILE | A | 56 | −1.391 | 94.639 | 9.500 | 1.00 | 65.47 | C |
| ATOM | 167 | CD1 | ILE | A | 56 | −2.240 | 95.670 | 10.281 | 1.00 | 66.61 | C |
| ATOM | 168 | CG2 | ILE | A | 56 | −1.969 | 95.273 | 7.149 | 1.00 | 65.46 | C |
| ATOM | 169 | C | ILE | A | 56 | 0.759 | 94.780 | 6.267 | 1.00 | 65.56 | C |
| ATOM | 170 | O | ILE | A | 56 | 1.422 | 95.805 | 6.155 | 1.00 | 65.96 | O |
| ATOM | 171 | N | ARG | A | 57 | 0.419 | 94.017 | 5.233 | 1.00 | 67.26 | N |
| ATOM | 172 | CA | ARG | A | 57 | 0.731 | 94.386 | 3.858 | 1.00 | 68.96 | C |
| ATOM | 173 | CB | ARG | A | 57 | 0.628 | 93.161 | 2.946 | 1.00 | 68.74 | C |
| ATOM | 174 | CG | ARG | A | 57 | 1.139 | 93.361 | 1.520 | 1.00 | 68.49 | C |
| ATOM | 175 | CD | ARG | A | 57 | 0.433 | 92.424 | 0.532 | 1.00 | 68.56 | C |
| ATOM | 176 | NE | ARG | A | 57 | 1.266 | 91.272 | 0.179 | 1.00 | 68.20 | N |
| ATOM | 177 | CZ | ARG | A | 57 | 0.777 | 90.086 | −0.208 | 1.00 | 68.80 | C |
| ATOM | 178 | NH1 | ARG | A | 57 | −0.551 | 89.870 | −0.291 | 1.00 | 69.12 | N |
| ATOM | 179 | NH2 | ARG | A | 57 | 1.616 | 89.106 | −0.517 | 1.00 | 69.04 | N |
| ATOM | 180 | C | ARG | A | 57 | −0.259 | 95.448 | 3.422 | 1.00 | 70.41 | C |
| ATOM | 181 | O | ARG | A | 57 | −1.430 | 95.146 | 3.171 | 1.00 | 70.64 | O |
| ATOM | 182 | N | VAL | A | 58 | 0.218 | 96.691 | 3.345 | 1.00 | 72.30 | N |
| ATOM | 183 | CA | VAL | A | 58 | −0.626 | 97.844 | 2.998 | 1.00 | 73.91 | C |
| ATOM | 184 | CB | VAL | A | 58 | 0.193 | 99.177 | 2.969 | 1.00 | 73.84 | C |
| ATOM | 185 | CG1 | VAL | A | 58 | −0.704 | 100.373 | 2.670 | 1.00 | 73.85 | C |
| ATOM | 186 | CG2 | VAL | A | 58 | 0.924 | 99.394 | 4.297 | 1.00 | 73.45 | C |
| ATOM | 187 | C | VAL | A | 58 | −1.348 | 97.602 | 1.666 | 1.00 | 74.98 | C |
| ATOM | 188 | O | VAL | A | 58 | −2.468 | 98.081 | 1.465 | 1.00 | 75.68 | O |
| ATOM | 189 | N | SER | A | 59 | −0.710 | 96.822 | 0.788 | 1.00 | 75.93 | N |
| ATOM | 190 | CA | SER | A | 59 | −1.268 | 96.456 | −0.521 | 1.00 | 76.51 | C |
| ATOM | 191 | CB | SER | A | 59 | −0.255 | 95.617 | −1.320 | 1.00 | 76.86 | C |
| ATOM | 192 | OG | SER | A | 59 | 1.103 | 96.061 | −1.049 | 1.00 | 78.49 | O |
| ATOM | 193 | C | SER | A | 59 | −2.617 | 95.721 | −0.460 | 1.00 | 76.40 | C |
| ATOM | 194 | O | SER | A | 59 | −3.382 | 95.775 | −1.422 | 1.00 | 76.81 | O |
| ATOM | 195 | N | ASP | A | 60 | −2.902 | 95.026 | 0.645 | 1.00 | 75.89 | N |
| ATOM | 196 | CA | ASP | A | 60 | −4.174 | 94.299 | 0.790 | 1.00 | 75.35 | C |
| ATOM | 197 | CB | ASP | A | 60 | −4.230 | 93.077 | −0.148 | 1.00 | 75.67 | C |
| ATOM | 198 | CG | ASP | A | 60 | −3.124 | 92.064 | 0.126 | 1.00 | 76.95 | C |
| ATOM | 199 | OD1 | ASP | A | 60 | −2.835 | 91.788 | 1.307 | 1.00 | 78.19 | O |
| ATOM | 200 | OD2 | ASP | A | 60 | −2.488 | 91.483 | −0.788 | 1.00 | 77.92 | O |
| ATOM | 201 | C | ASP | A | 60 | −4.543 | 93.872 | 2.217 | 1.00 | 74.33 | C |
| ATOM | 202 | O | ASP | A | 60 | −5.339 | 92.947 | 2.398 | 1.00 | 74.34 | O |
| ATOM | 203 | N | ASN | A | 61 | −3.965 | 94.541 | 3.215 | 1.00 | 72.99 | N |
| ATOM | 204 | CA | ASN | A | 61 | −4.194 | 94.219 | 4.633 | 1.00 | 71.45 | C |
| ATOM | 205 | CB | ASN | A | 61 | −5.599 | 94.651 | 5.074 | 1.00 | 72.10 | C |
| ATOM | 206 | CG | ASN | A | 61 | −5.790 | 96.158 | 5.026 | 1.00 | 73.42 | C |
| ATOM | 207 | OD1 | ASN | A | 61 | −5.333 | 96.885 | 5.926 | 1.00 | 74.58 | O |
| ATOM | 208 | ND2 | ASN | A | 61 | −6.471 | 96.636 | 3.975 | 1.00 | 74.28 | N |
| ATOM | 209 | C | ASN | A | 61 | −3.928 | 92.759 | 5.035 | 1.00 | 69.65 | C |
| ATOM | 210 | O | ASN | A | 61 | −4.535 | 92.242 | 5.975 | 1.00 | 69.76 | O |
| ATOM | 211 | N | LEU | A | 62 | −3.020 | 92.098 | 4.323 | 1.00 | 67.18 | N |
| ATOM | 212 | CA | LEU | A | 62 | −2.623 | 90.738 | 4.680 | 1.00 | 64.33 | C |
| ATOM | 213 | CB | LEU | A | 62 | −1.901 | 90.057 | 3.518 | 1.00 | 64.74 | C |
| ATOM | 214 | CG | LEU | A | 62 | −1.291 | 88.685 | 3.821 | 1.00 | 65.22 | C |
| ATOM | 215 | CD1 | LEU | A | 62 | −2.383 | 87.635 | 4.009 | 1.00 | 65.88 | C |
| ATOM | 216 | CD2 | LEU | A | 62 | −0.325 | 88.264 | 2.725 | 1.00 | 65.33 | C |
| ATOM | 217 | C | LEU | A | 62 | −1.698 | 90.766 | 5.883 | 1.00 | 61.89 | C |
| ATOM | 218 | O | LEU | A | 62 | −0.682 | 91.453 | 5.863 | 1.00 | 61.51 | O |
| ATOM | 219 | N | PRO | A | 63 | −2.044 | 90.010 | 6.920 | 1.00 | 59.57 | N |
| ATOM | 220 | CA | PRO | A | 63 | −1.164 | 89.840 | 8.083 | 1.00 | 57.75 | C |
| ATOM | 221 | CB | PRO | A | 63 | −1.963 | 88.888 | 8.983 | 1.00 | 57.73 | C |
| ATOM | 222 | CG | PRO | A | 63 | −3.376 | 89.106 | 8.573 | 1.00 | 58.58 | C |
| ATOM | 223 | CD | PRO | A | 63 | −3.303 | 89.261 | 7.080 | 1.00 | 59.38 | C |
| ATOM | 224 | C | PRO | A | 63 | 0.180 | 89.221 | 7.694 | 1.00 | 55.60 | C |
| ATOM | 225 | O | PRO | A | 63 | 0.211 | 88.163 | 7.075 | 1.00 | 55.56 | O |
| ATOM | 226 | N | VAL | A | 64 | 1.274 | 89.902 | 8.025 | 1.00 | 53.21 | N |
| ATOM | 227 | CA | VAL | A | 64 | 2.609 | 89.375 | 7.773 | 1.00 | 50.67 | C |
| ATOM | 228 | CB | VAL | A | 64 | 3.306 | 90.097 | 6.590 | 1.00 | 50.93 | C |
| ATOM | 229 | CG1 | VAL | A | 64 | 2.441 | 90.040 | 5.326 | 1.00 | 50.56 | C |
| ATOM | 230 | CG2 | VAL | A | 64 | 3.641 | 91.537 | 6.943 | 1.00 | 49.88 | C |
| ATOM | 231 | C | VAL | A | 64 | 3.492 | 89.445 | 9.025 | 1.00 | 49.15 | C |
| ATOM | 232 | O | VAL | A | 64 | 3.175 | 90.150 | 9.981 | 1.00 | 49.44 | O |
| ATOM | 233 | N | ALA | A | 65 | 4.587 | 88.692 | 9.015 | 1.00 | 46.68 | N |
| ATOM | 234 | CA | ALA | A | 65 | 5.604 | 88.774 | 10.046 | 1.00 | 44.84 | C |
| ATOM | 235 | CB | ALA | A | 65 | 5.881 | 87.384 | 10.654 | 1.00 | 45.04 | C |
| ATOM | 236 | C | ALA | A | 65 | 6.834 | 89.315 | 9.356 | 1.00 | 43.92 | C |
| ATOM | 237 | O | ALA | A | 65 | 7.123 | 88.912 | 8.218 | 1.00 | 43.40 | O |
| ATOM | 238 | N | ILE | A | 66 | 7.547 | 90.233 | 10.012 | 1.00 | 42.88 | N |
| ATOM | 239 | CA | ILE | A | 66 | 8.716 | 90.883 | 9.405 | 1.00 | 42.53 | C |
| ATOM | 240 | CB | ILE | A | 66 | 8.494 | 92.410 | 9.252 | 1.00 | 43.51 | C |
| ATOM | 241 | CG1 | ILE | A | 66 | 7.260 | 92.679 | 8.383 | 1.00 | 44.11 | C |
| ATOM | 242 | CD1 | ILE | A | 66 | 6.685 | 94.112 | 8.501 | 1.00 | 47.03 | C |
| ATOM | 243 | CG2 | ILE | A | 66 | 9.704 | 93.067 | 8.636 | 1.00 | 42.39 | C |
| ATOM | 244 | C | ILE | A | 66 | 9.958 | 90.572 | 10.214 | 1.00 | 42.61 | C |
| ATOM | 245 | O | ILE | A | 66 | 10.119 | 91.057 | 11.342 | 1.00 | 41.89 | O |
| ATOM | 246 | N | LYS | A | 67 | 10.820 | 89.731 | 9.641 | 1.00 | 41.21 | N |
| ATOM | 247 | CA | LYS | A | 67 | 11.971 | 89.193 | 10.353 | 1.00 | 41.66 | C |
| ATOM | 248 | CB | LYS | A | 67 | 12.052 | 87.664 | 10.164 | 1.00 | 41.05 | C |
| ATOM | 249 | CG | LYS | A | 67 | 13.288 | 87.013 | 10.761 | 1.00 | 42.61 | C |
| ATOM | 250 | CD | LYS | A | 67 | 13.165 | 85.495 | 10.666 | 1.00 | 44.83 | C |
| ATOM | 251 | CE | LYS | A | 67 | 14.213 | 84.780 | 11.488 | 1.00 | 46.29 | C |
| ATOM | 252 | NZ | LYS | A | 67 | 14.165 | 83.309 | 11.228 | 1.00 | 46.83 | N |
| ATOM | 253 | C | LYS | A | 67 | 13.243 | 89.833 | 9.867 | 1.00 | 41.57 | C |
| ATOM | 254 | O | LYS | A | 67 | 13.548 | 89.773 | 8.671 | 1.00 | 40.97 | O |
| ATOM | 255 | N | HIS | A | 68 | 13.988 | 90.415 | 10.807 | 1.00 | 41.97 | N |
| ATOM | 256 | CA | HIS | A | 68 | 15.254 | 91.087 | 10.553 | 1.00 | 43.17 | C |
| ATOM | 257 | CB | HIS | A | 68 | 15.343 | 92.419 | 11.318 | 1.00 | 42.46 | C |
| ATOM | 258 | CG | HIS | A | 68 | 14.352 | 93.440 | 10.858 | 1.00 | 40.77 | C |
| ATOM | 259 | ND1 | HIS | A | 68 | 13.018 | 93.384 | 11.203 | 1.00 | 43.58 | N |
| ATOM | 260 | CE1 | HIS | A | 68 | 12.376 | 94.393 | 10.640 | 1.00 | 41.67 | C |
| ATOM | 261 | NE2 | HIS | A | 68 | 13.247 | 95.100 | 9.942 | 1.00 | 41.02 | N |
| ATOM | 262 | CD2 | HIS | A | 68 | 14.489 | 94.522 | 10.062 | 1.00 | 37.93 | C |
| ATOM | 263 | C | HIS | A | 68 | 16.408 | 90.217 | 10.960 | 1.00 | 45.01 | C |
| ATOM | 264 | O | HIS | A | 68 | 16.466 | 89.744 | 12.089 | 1.00 | 44.97 | O |
| ATOM | 265 | N | VAL | A | 69 | 17.340 | 90.027 | 10.030 | 1.00 | 46.61 | N |
| ATOM | 266 | CA | VAL | A | 69 | 18.516 | 89.225 | 10.272 | 1.00 | 49.40 | C |
| ATOM | 267 | CB | VAL | A | 69 | 18.538 | 87.969 | 9.359 | 1.00 | 49.48 | C |
| ATOM | 268 | CG1 | VAL | A | 69 | 19.738 | 87.093 | 9.675 | 1.00 | 50.89 | C |
| ATOM | 269 | CG2 | VAL | A | 69 | 17.266 | 87.146 | 9.529 | 1.00 | 49.70 | C |
| ATOM | 270 | C | VAL | A | 69 | 19.746 | 90.103 | 10.038 | 1.00 | 51.36 | C |
| ATOM | 271 | O | VAL | A | 69 | 19.879 | 90.721 | 8.983 | 1.00 | 50.89 | O |
| ATOM | 272 | N | GLU | A | 70 | 20.634 | 90.162 | 11.026 | 1.00 | 53.97 | N |
| ATOM | 273 | CA | GLU | A | 70 | 21.870 | 90.924 | 10.896 | 1.00 | 57.27 | C |
| ATOM | 274 | CB | GLU | A | 70 | 22.480 | 91.219 | 12.272 | 1.00 | 57.98 | C |
| ATOM | 275 | CG | GLU | A | 70 | 21.674 | 92.205 | 13.105 | 1.00 | 61.81 | C |
| ATOM | 276 | CD | GLU | A | 70 | 22.524 | 93.240 | 13.839 | 1.00 | 66.09 | C |
| ATOM | 277 | OE1 | GLU | A | 70 | 21.982 | 93.928 | 14.744 | 1.00 | 67.00 | O |
| ATOM | 278 | OE2 | GLU | A | 70 | 23.729 | 93.377 | 13.518 | 1.00 | 68.05 | O |
| ATOM | 279 | C | GLU | A | 70 | 22.861 | 90.148 | 10.057 | 1.00 | 58.15 | C |
| ATOM | 280 | O | GLU | A | 70 | 23.115 | 88.977 | 10.332 | 1.00 | 57.86 | O |
| ATOM | 281 | N | LYS | A | 71 | 23.420 | 90.807 | 9.041 | 1.00 | 60.40 | N |
| ATOM | 282 | CA | LYS | A | 71 | 24.433 | 90.193 | 8.174 | 1.00 | 62.67 | C |
| ATOM | 283 | CB | LYS | A | 71 | 24.982 | 91.207 | 7.166 | 1.00 | 62.59 | C |
| ATOM | 284 | CG | LYS | A | 71 | 23.999 | 91.544 | 6.056 | 1.00 | 63.22 | C |
| ATOM | 285 | CD | LYS | A | 71 | 24.634 | 92.387 | 4.973 | 1.00 | 64.60 | C |
| ATOM | 286 | CE | LYS | A | 71 | 23.644 | 92.635 | 3.848 | 1.00 | 65.13 | C |
| ATOM | 287 | NZ | LYS | A | 71 | 24.159 | 93.586 | 2.831 | 1.00 | 66.01 | N |
| ATOM | 288 | C | LYS | A | 71 | 25.567 | 89.589 | 8.987 | 1.00 | 64.37 | C |
| ATOM | 289 | O | LYS | A | 71 | 25.990 | 88.462 | 8.737 | 1.00 | 64.24 | O |
| ATOM | 290 | N | ASP | A | 72 | 26.029 | 90.336 | 9.986 | 1.00 | 67.27 | N |
| ATOM | 291 | CA | ASP | A | 72 | 27.153 | 89.928 | 10.820 | 1.00 | 70.03 | C |
| ATOM | 292 | CB | ASP | A | 72 | 27.483 | 91.037 | 11.814 | 1.00 | 70.83 | C |
| ATOM | 293 | CG | ASP | A | 72 | 28.294 | 92.162 | 11.177 | 1.00 | 73.07 | C |
| ATOM | 294 | OD1 | ASP | A | 72 | 27.828 | 92.764 | 10.174 | 1.00 | 75.44 | O |
| ATOM | 295 | OD2 | ASP | A | 72 | 29.412 | 92.511 | 11.611 | 1.00 | 74.89 | O |
| ATOM | 296 | C | ASP | A | 72 | 26.923 | 88.614 | 11.551 | 1.00 | 71.40 | C |
| ATOM | 297 | O | ASP | A | 72 | 27.875 | 87.895 | 11.851 | 1.00 | 71.59 | O |
| ATOM | 298 | N | ARG | A | 73 | 25.658 | 88.287 | 11.805 | 1.00 | 73.23 | N |
| ATOM | 299 | CA | ARG | A | 73 | 25.304 | 87.068 | 12.540 | 1.00 | 74.86 | C |
| ATOM | 300 | CB | ARG | A | 73 | 24.241 | 87.380 | 13.602 | 1.00 | 75.53 | C |
| ATOM | 301 | CG | ARG | A | 73 | 24.741 | 88.293 | 14.718 | 1.00 | 79.03 | C |
| ATOM | 302 | CD | ARG | A | 73 | 23.959 | 88.151 | 16.043 | 1.00 | 84.58 | C |
| ATOM | 303 | NE | ARG | A | 73 | 23.692 | 86.752 | 16.394 | 1.00 | 88.34 | N |
| ATOM | 304 | CZ | ARG | A | 73 | 24.598 | 85.901 | 16.878 | 1.00 | 89.85 | C |
| ATOM | 305 | NH1 | ARG | A | 73 | 25.857 | 86.293 | 17.083 | 1.00 | 90.48 | N |
| ATOM | 306 | NH2 | ARG | A | 73 | 24.239 | 84.650 | 17.161 | 1.00 | 90.61 | N |
| ATOM | 307 | C | ARG | A | 73 | 24.839 | 85.929 | 11.630 | 1.00 | 75.02 | C |
| ATOM | 308 | O | ARG | A | 73 | 24.067 | 85.067 | 12.054 | 1.00 | 75.13 | O |
| ATOM | 309 | N | ILE | A | 74 | 25.321 | 85.926 | 10.386 | 1.00 | 75.26 | N |
| ATOM | 310 | CA | ILE | A | 74 | 24.969 | 84.885 | 9.420 | 1.00 | 75.36 | C |
| ATOM | 311 | CB | ILE | A | 74 | 24.359 | 85.503 | 8.127 | 1.00 | 75.17 | C |
| ATOM | 312 | CG1 | ILE | A | 74 | 23.188 | 86.425 | 8.465 | 1.00 | 74.84 | C |
| ATOM | 313 | CD1 | ILE | A | 74 | 22.660 | 87.204 | 7.280 | 1.00 | 75.37 | C |
| ATOM | 314 | CG2 | ILE | A | 74 | 23.893 | 84.408 | 7.166 | 1.00 | 74.86 | C |
| ATOM | 315 | C | ILE | A | 74 | 26.189 | 84.022 | 9.088 | 1.00 | 75.92 | C |
| ATOM | 316 | O | ILE | A | 74 | 27.201 | 84.519 | 8.578 | 1.00 | 76.08 | O |
| ATOM | 317 | N | SER | A | 75 | 26.090 | 82.730 | 9.386 | 1.00 | 76.26 | N |
| ATOM | 318 | CA | SER | A | 75 | 27.155 | 81.784 | 9.072 | 1.00 | 76.63 | C |
| ATOM | 319 | CB | SER | A | 75 | 27.184 | 80.641 | 10.094 | 1.00 | 77.05 | C |
| ATOM | 320 | OG | SER | A | 75 | 26.007 | 79.839 | 10.009 | 1.00 | 78.24 | O |
| ATOM | 321 | C | SER | A | 75 | 26.990 | 81.226 | 7.660 | 1.00 | 76.35 | C |
| ATOM | 322 | O | SER | A | 75 | 27.918 | 81.285 | 6.855 | 1.00 | 76.40 | O |
| ATOM | 323 | N | ASP | A | 76 | 25.798 | 80.703 | 7.372 | 1.00 | 75.95 | N |
| ATOM | 324 | CA | ASP | A | 76 | 25.512 | 80.025 | 6.109 | 1.00 | 75.64 | C |
| ATOM | 325 | CB | ASP | A | 76 | 24.528 | 78.875 | 6.332 | 1.00 | 76.36 | C |
| ATOM | 326 | CG | ASP | A | 76 | 25.112 | 77.756 | 7.157 | 1.00 | 78.38 | C |
| ATOM | 327 | OD1 | ASP | A | 76 | 25.828 | 76.906 | 6.579 | 1.00 | 80.43 | O |
| ATOM | 328 | OD2 | ASP | A | 76 | 24.900 | 77.642 | 8.391 | 1.00 | 81.66 | O |
| ATOM | 329 | C | ASP | A | 76 | 24.948 | 80.952 | 5.043 | 1.00 | 74.58 | C |
| ATOM | 330 | O | ASP | A | 76 | 23.946 | 81.635 | 5.262 | 1.00 | 74.37 | O |
| ATOM | 331 | N | TRP | A | 77 | 25.592 | 80.945 | 3.879 | 1.00 | 73.73 | N |
| ATOM | 332 | CA | TRP | A | 77 | 25.148 | 81.728 | 2.730 | 1.00 | 72.88 | C |
| ATOM | 333 | CB | TRP | A | 77 | 26.159 | 82.826 | 2.398 | 1.00 | 72.08 | C |
| ATOM | 334 | CG | TRP | A | 77 | 26.345 | 83.854 | 3.455 | 1.00 | 68.72 | C |
| ATOM | 335 | CD1 | TRP | A | 77 | 27.105 | 83.748 | 4.582 | 1.00 | 67.14 | C |
| ATOM | 336 | NE1 | TRP | A | 77 | 27.038 | 84.911 | 5.313 | 1.00 | 66.79 | N |
| ATOM | 337 | CE2 | TRP | A | 77 | 26.228 | 85.800 | 4.657 | 1.00 | 66.47 | C |
| ATOM | 338 | CD2 | TRP | A | 77 | 25.776 | 85.163 | 3.478 | 1.00 | 66.40 | C |
| ATOM | 339 | CE3 | TRP | A | 77 | 24.926 | 85.870 | 2.620 | 1.00 | 65.70 | C |
| ATOM | 340 | CZ3 | TRP | A | 77 | 24.557 | 87.169 | 2.958 | 1.00 | 65.66 | C |
| ATOM | 341 | CH2 | TRP | A | 77 | 25.023 | 87.770 | 4.139 | 1.00 | 65.79 | C |
| ATOM | 342 | CZ2 | TRP | A | 77 | 25.858 | 87.104 | 5.000 | 1.00 | 65.98 | C |
| ATOM | 343 | C | TRP | A | 77 | 25.020 | 80.808 | 1.529 | 1.00 | 73.58 | C |
| ATOM | 344 | O | TRP | A | 77 | 25.727 | 79.802 | 1.434 | 1.00 | 73.41 | O |
| ATOM | 345 | N | GLY | A | 78 | 24.127 | 81.159 | 0.610 | 1.00 | 74.27 | N |
| ATOM | 346 | CA | GLY | A | 78 | 23.959 | 80.407 | −0.623 | 1.00 | 75.77 | C |
| ATOM | 347 | C | GLY | A | 78 | 23.491 | 81.313 | −1.740 | 1.00 | 77.06 | C |
| ATOM | 348 | O | GLY | A | 78 | 23.426 | 82.534 | −1.567 | 1.00 | 77.12 | O |
| ATOM | 349 | N | GLU | A | 79 | 23.157 | 80.725 | −2.887 | 1.00 | 78.52 | N |
| ATOM | 350 | CA | GLU | A | 79 | 22.685 | 81.517 | −4.022 | 1.00 | 80.32 | C |
| ATOM | 351 | CB | GLU | A | 79 | 23.710 | 81.532 | −5.170 | 1.00 | 80.86 | C |
| ATOM | 352 | CG | GLU | A | 79 | 24.083 | 80.152 | −5.723 | 1.00 | 83.43 | C |
| ATOM | 353 | CD | GLU | A | 79 | 24.713 | 80.234 | −7.108 | 1.00 | 85.86 | C |
| ATOM | 354 | OE1 | GLU | A | 79 | 25.813 | 80.822 | −7.235 | 1.00 | 86.43 | O |
| ATOM | 355 | OE2 | GLU | A | 79 | 24.107 | 79.708 | −8.071 | 1.00 | 86.75 | O |
| ATOM | 356 | C | GLU | A | 79 | 21.311 | 81.091 | −4.518 | 1.00 | 80.80 | C |
| ATOM | 357 | O | GLU | A | 79 | 20.948 | 79.917 | −4.453 | 1.00 | 80.46 | O |
| ATOM | 358 | N | LEU | A | 80 | 20.558 | 82.069 | −5.012 | 1.00 | 81.81 | N |
| ATOM | 359 | CA | LEU | A | 80 | 19.233 | 81.837 | −5.582 | 1.00 | 82.81 | C |
| ATOM | 360 | CB | LEU | A | 80 | 18.441 | 83.152 | −5.596 | 1.00 | 82.78 | C |
| ATOM | 361 | CG | LEU | A | 80 | 18.289 | 83.870 | −4.254 | 1.00 | 83.30 | C |
| ATOM | 362 | CD1 | LEU | A | 80 | 17.545 | 85.189 | −4.432 | 1.00 | 83.40 | C |
| ATOM | 363 | CD2 | LEU | A | 80 | 17.588 | 82.965 | −3.238 | 1.00 | 83.57 | C |
| ATOM | 364 | C | LEU | A | 80 | 19.343 | 81.256 | −6.998 | 1.00 | 83.29 | C |
| ATOM | 365 | O | LEU | A | 80 | 20.440 | 81.256 | −7.570 | 1.00 | 83.54 | O |
| ATOM | 366 | N | PRO | A | 81 | 18.235 | 80.753 | −7.567 | 1.00 | 83.78 | N |
| ATOM | 367 | CA | PRO | A | 81 | 18.221 | 80.340 | −8.986 | 1.00 | 83.97 | C |
| ATOM | 368 | CB | PRO | A | 81 | 16.758 | 79.937 | −9.218 | 1.00 | 83.94 | C |
| ATOM | 369 | CG | PRO | A | 81 | 16.267 | 79.535 | −7.871 | 1.00 | 84.00 | C |
| ATOM | 370 | CD | PRO | A | 81 | 16.927 | 80.513 | −6.923 | 1.00 | 83.86 | C |
| ATOM | 371 | C | PRO | A | 81 | 18.623 | 81.488 | −9.926 | 1.00 | 84.09 | C |
| ATOM | 372 | O | PRO | A | 81 | 18.910 | 81.267 | −11.102 | 1.00 | 84.07 | O |
| ATOM | 373 | N | ASN | A | 82 | 18.644 | 82.700 | −9.376 | 1.00 | 84.20 | N |
| ATOM | 374 | CA | ASN | A | 82 | 19.070 | 83.916 | −10.064 | 1.00 | 83.96 | C |
| ATOM | 375 | CB | ASN | A | 82 | 18.276 | 85.106 | −9.492 | 1.00 | 84.25 | C |
| ATOM | 376 | CG | ASN | A | 82 | 18.738 | 86.449 | −10.026 | 1.00 | 85.14 | C |
| ATOM | 377 | OD1 | ASN | A | 82 | 18.869 | 86.643 | −11.241 | 1.00 | 85.89 | O |
| ATOM | 378 | ND2 | ASN | A | 82 | 18.979 | 87.393 | −9.115 | 1.00 | 84.89 | N |
| ATOM | 379 | C | ASN | A | 82 | 20.586 | 84.137 | −9.935 | 1.00 | 83.40 | C |
| ATOM | 380 | O | ASN | A | 82 | 21.190 | 84.889 | −10.709 | 1.00 | 83.24 | O |
| ATOM | 381 | N | GLY | A | 83 | 21.191 | 83.461 | −8.958 | 1.00 | 82.90 | N |
| ATOM | 382 | CA | GLY | A | 83 | 22.597 | 83.634 | −8.626 | 1.00 | 82.10 | C |
| ATOM | 383 | C | GLY | A | 83 | 22.845 | 84.924 | −7.863 | 1.00 | 81.49 | C |
| ATOM | 384 | O | GLY | A | 83 | 23.382 | 85.883 | −8.430 | 1.00 | 81.74 | O |
| ATOM | 385 | N | THR | A | 84 | 22.437 | 84.944 | −6.590 | 1.00 | 80.61 | N |
| ATOM | 386 | CA | THR | A | 84 | 22.609 | 86.097 | −5.687 | 1.00 | 79.41 | C |
| ATOM | 387 | CB | THR | A | 84 | 21.290 | 86.893 | −5.543 | 1.00 | 79.60 | C |
| ATOM | 388 | OG1 | THR | A | 84 | 20.718 | 87.127 | −6.836 | 1.00 | 80.05 | O |
| ATOM | 389 | CG2 | THR | A | 84 | 21.561 | 88.322 | −5.007 | 1.00 | 79.91 | C |
| ATOM | 390 | C | THR | A | 84 | 23.081 | 85.643 | −4.302 | 1.00 | 78.07 | C |
| ATOM | 391 | O | THR | A | 84 | 22.728 | 84.557 | −3.841 | 1.00 | 78.47 | O |
| ATOM | 392 | N | ARG | A | 85 | 23.866 | 86.489 | −3.643 | 1.00 | 75.99 | N |
| ATOM | 393 | CA | ARG | A | 85 | 24.443 | 86.177 | −2.338 | 1.00 | 73.77 | C |
| ATOM | 394 | CB | ARG | A | 85 | 25.768 | 86.943 | −2.184 | 1.00 | 74.21 | C |
| ATOM | 395 | CG | ARG | A | 85 | 26.453 | 86.829 | −0.833 | 1.00 | 75.15 | C |
| ATOM | 396 | CD | ARG | A | 85 | 27.404 | 85.638 | −0.725 | 1.00 | 75.91 | C |
| ATOM | 397 | NE | ARG | A | 85 | 28.213 | 85.731 | 0.487 | 1.00 | 76.30 | N |
| ATOM | 398 | CZ | ARG | A | 85 | 28.957 | 84.739 | 0.972 | 1.00 | 77.02 | C |
| ATOM | 399 | NH1 | ARG | A | 85 | 29.005 | 83.560 | 0.352 | 1.00 | 76.24 | N |
| ATOM | 400 | NH2 | ARG | A | 85 | 29.655 | 84.929 | 2.086 | 1.00 | 77.19 | N |
| ATOM | 401 | C | ARG | A | 85 | 23.457 | 86.502 | −1.199 | 1.00 | 71.72 | C |
| ATOM | 402 | O | ARG | A | 85 | 23.415 | 87.632 | −0.696 | 1.00 | 71.91 | O |
| ATOM | 403 | N | VAL | A | 86 | 22.653 | 85.514 | −0.809 | 1.00 | 68.61 | N |
| ATOM | 404 | CA | VAL | A | 86 | 21.660 | 85.693 | 0.262 | 1.00 | 65.46 | C |
| ATOM | 405 | CB | VAL | A | 86 | 20.191 | 85.642 | −0.265 | 1.00 | 65.32 | C |
| ATOM | 406 | CG1 | VAL | A | 86 | 19.977 | 86.633 | −1.394 | 1.00 | 64.79 | C |
| ATOM | 407 | CG2 | VAL | A | 86 | 19.822 | 84.250 | −0.709 | 1.00 | 65.00 | C |
| ATOM | 408 | C | VAL | A | 86 | 21.866 | 84.656 | 1.372 | 1.00 | 63.06 | C |
| ATOM | 409 | O | VAL | A | 86 | 22.543 | 83.649 | 1.144 | 1.00 | 63.20 | O |
| ATOM | 410 | N | PRO | A | 87 | 21.301 | 84.887 | 2.563 | 1.00 | 60.50 | N |
| ATOM | 411 | CA | PRO | A | 87 | 21.399 | 83.907 | 3.649 | 1.00 | 58.23 | C |
| ATOM | 412 | CB | PRO | A | 87 | 20.570 | 84.544 | 4.774 | 1.00 | 58.22 | C |
| ATOM | 413 | CG | PRO | A | 87 | 20.590 | 85.999 | 4.478 | 1.00 | 59.47 | C |
| ATOM | 414 | CD | PRO | A | 87 | 20.535 | 86.082 | 2.986 | 1.00 | 60.10 | C |
| ATOM | 415 | C | PRO | A | 87 | 20.797 | 82.564 | 3.237 | 1.00 | 56.09 | C |
| ATOM | 416 | O | PRO | A | 87 | 19.802 | 82.524 | 2.513 | 1.00 | 55.24 | O |
| ATOM | 417 | N | MET | A | 88 | 21.416 | 81.479 | 3.681 | 1.00 | 54.23 | N |
| ATOM | 418 | CA | MET | A | 88 | 20.866 | 80.142 | 3.458 | 1.00 | 53.19 | C |
| ATOM | 419 | CB | MET | A | 88 | 21.638 | 79.111 | 4.295 | 1.00 | 54.18 | C |
| ATOM | 420 | CG | MET | A | 88 | 21.273 | 77.645 | 4.025 | 1.00 | 57.50 | C |
| ATOM | 421 | SD | MET | A | 88 | 21.341 | 77.213 | 2.247 | 1.00 | 65.32 | S |
| ATOM | 422 | CE | MET | A | 88 | 23.113 | 77.148 | 2.002 | 1.00 | 62.88 | C |
| ATOM | 423 | C | MET | A | 88 | 19.363 | 80.103 | 3.775 | 1.00 | 50.99 | C |
| ATOM | 424 | O | MET | A | 88 | 18.565 | 79.594 | 2.979 | 1.00 | 49.59 | O |
| ATOM | 425 | N | GLU | A | 89 | 18.982 | 80.688 | 4.918 | 1.00 | 48.97 | N |
| ATOM | 426 | CA | GLU | A | 89 | 17.575 | 80.754 | 5.317 | 1.00 | 46.86 | C |
| ATOM | 427 | CB | GLU | A | 89 | 17.392 | 81.686 | 6.522 | 1.00 | 45.85 | C |
| ATOM | 428 | CG | GLU | A | 89 | 15.944 | 81.803 | 6.991 | 1.00 | 45.51 | C |
| ATOM | 429 | CD | GLU | A | 89 | 15.803 | 82.541 | 8.303 | 1.00 | 44.03 | C |
| ATOM | 430 | OE1 | GLU | A | 89 | 16.819 | 83.008 | 8.856 | 1.00 | 47.34 | O |
| ATOM | 431 | OE2 | GLU | A | 89 | 14.671 | 82.638 | 8.790 | 1.00 | 44.02 | O |
| ATOM | 432 | C | GLU | A | 89 | 16.653 | 81.168 | 4.171 | 1.00 | 46.50 | C |
| ATOM | 433 | O | GLU | A | 89 | 15.612 | 80.548 | 3.962 | 1.00 | 46.41 | O |
| ATOM | 434 | N | VAL | A | 90 | 17.031 | 82.215 | 3.429 | 1.00 | 46.05 | N |
| ATOM | 435 | CA | VAL | A | 90 | 16.243 | 82.669 | 2.275 | 1.00 | 45.86 | C |
| ATOM | 436 | CB | VAL | A | 90 | 16.759 | 84.018 | 1.725 | 1.00 | 46.25 | C |
| ATOM | 437 | CG1 | VAL | A | 90 | 15.966 | 84.431 | 0.491 | 1.00 | 45.50 | C |
| ATOM | 438 | CG2 | VAL | A | 90 | 16.663 | 85.102 | 2.800 | 1.00 | 46.57 | C |
| ATOM | 439 | C | VAL | A | 90 | 16.234 | 81.639 | 1.137 | 1.00 | 45.66 | C |
| ATOM | 440 | O | VAL | A | 90 | 15.210 | 81.399 | 0.525 | 1.00 | 45.75 | O |
| ATOM | 441 | N | VAL | A | 91 | 17.389 | 81.053 | 0.851 | 1.00 | 45.99 | N |
| ATOM | 442 | CA | VAL | A | 91 | 17.490 | 80.034 | −0.197 | 1.00 | 46.17 | C |
| ATOM | 443 | CB | VAL | A | 91 | 18.913 | 79.465 | −0.279 | 1.00 | 46.54 | C |
| ATOM | 444 | CG1 | VAL | A | 91 | 18.975 | 78.292 | −1.284 | 1.00 | 47.68 | C |
| ATOM | 445 | CG2 | VAL | A | 91 | 19.892 | 80.556 | −0.674 | 1.00 | 48.34 | C |
| ATOM | 446 | C | VAL | A | 91 | 16.496 | 78.909 | 0.094 | 1.00 | 44.94 | C |
| ATOM | 447 | O | VAL | A | 91 | 15.631 | 78.603 | −0.729 | 1.00 | 45.36 | O |
| ATOM | 448 | N | LEU | A | 92 | 16.591 | 78.352 | 1.302 | 1.00 | 43.94 | N |
| ATOM | 449 | CA | LEU | A | 92 | 15.704 | 77.260 | 1.749 | 1.00 | 42.33 | C |
| ATOM | 450 | CB | LEU | A | 92 | 16.106 | 76.772 | 3.137 | 1.00 | 40.99 | C |
| ATOM | 451 | CG | LEU | A | 92 | 17.577 | 76.417 | 3.316 | 1.00 | 40.75 | C |
| ATOM | 452 | CD1 | LEU | A | 92 | 17.798 | 75.867 | 4.711 | 1.00 | 37.63 | C |
| ATOM | 453 | CD2 | LEU | A | 92 | 18.061 | 75.410 | 2.247 | 1.00 | 40.38 | C |
| ATOM | 454 | C | LEU | A | 92 | 14.245 | 77.641 | 1.742 | 1.00 | 42.09 | C |
| ATOM | 455 | O | LEU | A | 92 | 13.401 | 76.888 | 1.243 | 1.00 | 41.96 | O |
| ATOM | 456 | N | LEU | A | 93 | 13.936 | 78.812 | 2.289 | 1.00 | 42.17 | N |
| ATOM | 457 | CA | LEU | A | 93 | 12.556 | 79.280 | 2.328 | 1.00 | 43.42 | C |
| ATOM | 458 | CB | LEU | A | 93 | 12.461 | 80.632 | 3.051 | 1.00 | 42.52 | C |
| ATOM | 459 | CG | LEU | A | 93 | 12.416 | 80.645 | 4.589 | 1.00 | 42.30 | C |
| ATOM | 460 | CD1 | LEU | A | 93 | 12.576 | 82.074 | 5.095 | 1.00 | 39.64 | C |
| ATOM | 461 | CD2 | LEU | A | 93 | 11.117 | 80.069 | 5.107 | 1.00 | 39.20 | C |
| ATOM | 462 | C | LEU | A | 93 | 11.947 | 79.382 | 0.921 | 1.00 | 44.51 | C |
| ATOM | 463 | O | LEU | A | 93 | 10.823 | 78.940 | 0.691 | 1.00 | 44.42 | O |
| ATOM | 464 | N | LYS | A | 94 | 12.690 | 79.981 | −0.012 | 1.00 | 46.08 | N |
| ATOM | 465 | CA | LYS | A | 94 | 12.222 | 80.098 | −1.391 | 1.00 | 47.69 | C |
| ATOM | 466 | CB | LYS | A | 94 | 13.266 | 80.807 | −2.254 | 1.00 | 48.80 | C |
| ATOM | 467 | CG | LYS | A | 94 | 13.146 | 82.329 | −2.195 | 1.00 | 52.89 | C |
| ATOM | 468 | CD | LYS | A | 94 | 14.133 | 83.009 | −3.126 | 1.00 | 57.20 | C |
| ATOM | 469 | CE | LYS | A | 94 | 13.761 | 82.810 | −4.598 | 1.00 | 58.87 | C |
| ATOM | 470 | NZ | LYS | A | 94 | 12.411 | 83.360 | −4.919 | 1.00 | 60.23 | N |
| ATOM | 471 | C | LYS | A | 94 | 11.903 | 78.730 | −1.981 | 1.00 | 47.52 | C |
| ATOM | 472 | O | LYS | A | 94 | 10.870 | 78.564 | −2.633 | 1.00 | 48.02 | O |
| ATOM | 473 | N | LYS | A | 95 | 12.792 | 77.766 | −1.733 | 1.00 | 47.55 | N |
| ATOM | 474 | CA | LYS | A | 95 | 12.615 | 76.380 | −2.185 | 1.00 | 48.35 | C |
| ATOM | 475 | CB | LYS | A | 95 | 13.836 | 75.536 | −1.829 | 1.00 | 48.23 | C |
| ATOM | 476 | CG | LYS | A | 95 | 15.023 | 75.801 | −2.747 | 1.00 | 48.74 | C |
| ATOM | 477 | CD | LYS | A | 95 | 16.293 | 75.188 | −2.212 | 1.00 | 50.92 | C |
| ATOM | 478 | CE | LYS | A | 95 | 16.392 | 73.705 | −2.529 | 1.00 | 53.76 | C |
| ATOM | 479 | NZ | LYS | A | 95 | 16.339 | 73.414 | −3.999 | 1.00 | 55.34 | N |
| ATOM | 480 | C | LYS | A | 95 | 11.351 | 75.706 | −1.659 | 1.00 | 48.52 | C |
| ATOM | 481 | O | LYS | A | 95 | 10.770 | 74.872 | −2.358 | 1.00 | 48.90 | O |
| ATOM | 482 | N | VAL | A | 96 | 10.921 | 76.056 | −0.444 | 1.00 | 48.20 | N |
| ATOM | 483 | CA | VAL | A | 96 | 9.759 | 75.395 | 0.149 | 1.00 | 48.68 | C |
| ATOM | 484 | CB | VAL | A | 96 | 10.001 | 74.989 | 1.620 | 1.00 | 48.64 | C |
| ATOM | 485 | CG1 | VAL | A | 96 | 11.105 | 73.977 | 1.718 | 1.00 | 45.61 | C |
| ATOM | 486 | CG2 | VAL | A | 96 | 10.301 | 76.238 | 2.498 | 1.00 | 47.01 | C |
| ATOM | 487 | C | VAL | A | 96 | 8.469 | 76.211 | 0.082 | 1.00 | 50.79 | C |
| ATOM | 488 | O | VAL | A | 96 | 7.412 | 75.751 | 0.544 | 1.00 | 50.14 | O |
| ATOM | 489 | N | SER | A | 97 | 8.547 | 77.419 | −0.476 | 1.00 | 52.75 | N |
| ATOM | 490 | CA | SER | A | 97 | 7.384 | 78.301 | −0.523 | 1.00 | 55.97 | C |
| ATOM | 491 | CB | SER | A | 97 | 7.820 | 79.751 | −0.306 | 1.00 | 56.04 | C |
| ATOM | 492 | OG | SER | A | 97 | 8.364 | 79.912 | 0.999 | 1.00 | 53.48 | O |
| ATOM | 493 | C | SER | A | 97 | 6.572 | 78.124 | −1.816 | 1.00 | 58.83 | C |
| ATOM | 494 | O | SER | A | 97 | 7.097 | 78.270 | −2.921 | 1.00 | 60.33 | O |
| ATOM | 495 | N | SER | A | 98 | 5.294 | 77.767 | −1.667 | 1.00 | 61.85 | N |
| ATOM | 496 | CA | SER | A | 98 | 4.397 | 77.478 | −2.805 | 1.00 | 63.61 | C |
| ATOM | 497 | CB | SER | A | 98 | 5.081 | 76.573 | −3.822 | 1.00 | 63.67 | C |
| ATOM | 498 | OG | SER | A | 98 | 5.317 | 75.300 | −3.246 | 1.00 | 63.53 | O |
| ATOM | 499 | C | SER | A | 98 | 3.120 | 76.797 | −2.304 | 1.00 | 65.08 | C |
| ATOM | 500 | O | SER | A | 98 | 2.764 | 76.902 | −1.125 | 1.00 | 65.04 | O |
| ATOM | 501 | N | GLY | A | 99 | 2.442 | 76.091 | −3.204 | 1.00 | 66.21 | N |
| ATOM | 502 | CA | GLY | A | 99 | 1.192 | 75.403 | −2.892 | 1.00 | 67.46 | C |
| ATOM | 503 | C | GLY | A | 99 | 0.948 | 74.924 | −1.464 | 1.00 | 67.88 | C |
| ATOM | 504 | O | GLY | A | 99 | −0.086 | 75.258 | −0.860 | 1.00 | 68.29 | O |
| ATOM | 505 | N | PHE | A | 100 | 1.877 | 74.127 | −0.924 | 1.00 | 68.05 | N |
| ATOM | 506 | CA | PHE | A | 100 | 1.723 | 73.626 | 0.436 | 1.00 | 67.53 | C |
| ATOM | 507 | CB | PHE | A | 100 | 2.873 | 72.738 | 0.871 | 1.00 | 68.29 | C |
| ATOM | 508 | CG | PHE | A | 100 | 2.530 | 71.844 | 2.047 | 1.00 | 69.62 | C |
| ATOM | 509 | CD1 | PHE | A | 100 | 1.249 | 71.278 | 2.168 | 1.00 | 70.09 | C |
| ATOM | 510 | CE1 | PHE | A | 100 | 0.933 | 70.435 | 3.245 | 1.00 | 70.04 | C |
| ATOM | 511 | CZ | PHE | A | 100 | 1.906 | 70.147 | 4.214 | 1.00 | 69.68 | C |
| ATOM | 512 | CE2 | PHE | A | 100 | 3.181 | 70.698 | 4.103 | 1.00 | 69.67 | C |
| ATOM | 513 | CD2 | PHE | A | 100 | 3.488 | 71.552 | 3.025 | 1.00 | 70.39 | C |
| ATOM | 514 | C | PHE | A | 100 | 1.617 | 74.720 | 1.453 | 1.00 | 66.54 | C |
| ATOM | 515 | O | PHE | A | 100 | 1.946 | 75.873 | 1.193 | 1.00 | 68.10 | O |
| ATOM | 516 | N | SER | A | 101 | 1.174 | 74.341 | 2.637 | 1.00 | 64.56 | N |
| ATOM | 517 | CA | SER | A | 101 | 0.954 | 75.295 | 3.693 | 1.00 | 61.98 | C |
| ATOM | 518 | CB | SER | A | 101 | −0.524 | 75.693 | 3.717 | 1.00 | 62.34 | C |
| ATOM | 519 | OG | SER | A | 101 | −1.344 | 74.533 | 3.712 | 1.00 | 64.11 | O |
| ATOM | 520 | C | SER | A | 101 | 1.379 | 74.726 | 5.036 | 1.00 | 59.07 | C |
| ATOM | 521 | O | SER | A | 101 | 0.982 | 75.260 | 6.087 | 1.00 | 60.11 | O |
| ATOM | 522 | N | GLY | A | 102 | 2.170 | 73.649 | 5.013 | 1.00 | 55.16 | N |
| ATOM | 523 | CA | GLY | A | 102 | 2.732 | 73.096 | 6.245 | 1.00 | 49.51 | C |
| ATOM | 524 | C | GLY | A | 102 | 3.986 | 73.857 | 6.676 | 1.00 | 46.58 | C |
| ATOM | 525 | O | GLY | A | 102 | 4.576 | 73.592 | 7.726 | 1.00 | 43.90 | O |
| ATOM | 526 | N | VAL | A | 103 | 4.411 | 74.794 | 5.840 | 1.00 | 44.62 | N |
| ATOM | 527 | CA | VAL | A | 103 | 5.521 | 75.673 | 6.162 | 1.00 | 44.58 | C |
| ATOM | 528 | CB | VAL | A | 103 | 6.738 | 75.384 | 5.255 | 1.00 | 44.89 | C |
| ATOM | 529 | CG1 | VAL | A | 103 | 7.832 | 76.366 | 5.505 | 1.00 | 46.40 | C |
| ATOM | 530 | CG2 | VAL | A | 103 | 7.282 | 73.964 | 5.503 | 1.00 | 45.56 | C |
| ATOM | 531 | C | VAL | A | 103 | 5.057 | 77.124 | 6.013 | 1.00 | 43.92 | C |
| ATOM | 532 | O | VAL | A | 103 | 4.370 | 77.458 | 5.049 | 1.00 | 43.86 | O |
| ATOM | 533 | N | ILE | A | 104 | 5.427 | 77.982 | 6.961 | 1.00 | 43.35 | N |
| ATOM | 534 | CA | ILE | A | 104 | 5.168 | 79.411 | 6.824 | 1.00 | 42.66 | C |
| ATOM | 535 | CB | ILE | A | 104 | 5.520 | 80.165 | 8.122 | 1.00 | 43.40 | C |
| ATOM | 536 | CG1 | ILE | A | 104 | 4.339 | 80.057 | 9.077 | 1.00 | 44.47 | C |
| ATOM | 537 | CD1 | ILE | A | 104 | 4.527 | 80.787 | 10.332 | 1.00 | 50.46 | C |
| ATOM | 538 | CG2 | ILE | A | 104 | 5.877 | 81.660 | 7.853 | 1.00 | 41.37 | C |
| ATOM | 539 | C | ILE | A | 104 | 5.961 | 79.925 | 5.622 | 1.00 | 42.85 | C |
| ATOM | 540 | O | ILE | A | 104 | 7.184 | 79.743 | 5.536 | 1.00 | 41.68 | O |
| ATOM | 541 | N | ARG | A | 105 | 5.241 | 80.535 | 4.691 | 1.00 | 42.70 | N |
| ATOM | 542 | CA | ARG | A | 105 | 5.807 | 80.875 | 3.395 | 1.00 | 44.62 | C |
| ATOM | 543 | CB | ARG | A | 105 | 4.690 | 80.815 | 2.360 | 1.00 | 46.35 | C |
| ATOM | 544 | CG | ARG | A | 105 | 5.065 | 81.242 | 0.972 | 1.00 | 53.53 | C |
| ATOM | 545 | CD | ARG | A | 105 | 4.460 | 80.351 | −0.099 | 1.00 | 61.61 | C |
| ATOM | 546 | NE | ARG | A | 105 | 3.071 | 80.019 | 0.185 | 1.00 | 65.80 | N |
| ATOM | 547 | CZ | ARG | A | 105 | 2.156 | 79.818 | −0.763 | 1.00 | 68.07 | C |
| ATOM | 548 | NH1 | ARG | A | 105 | 2.489 | 79.894 | −2.060 | 1.00 | 69.01 | N |
| ATOM | 549 | NH2 | ARG | A | 105 | 0.907 | 79.535 | −0.414 | 1.00 | 68.72 | N |
| ATOM | 550 | C | ARG | A | 105 | 6.464 | 82.255 | 3.412 | 1.00 | 43.35 | C |
| ATOM | 551 | O | ARG | A | 105 | 5.955 | 83.180 | 4.045 | 1.00 | 40.91 | O |
| ATOM | 552 | N | LEU | A | 106 | 7.597 | 82.359 | 2.722 | 1.00 | 42.75 | N |
| ATOM | 553 | CA | LEU | A | 106 | 8.288 | 83.624 | 2.481 | 1.00 | 43.28 | C |
| ATOM | 554 | CB | LEU | A | 106 | 9.746 | 83.349 | 2.126 | 1.00 | 42.30 | C |
| ATOM | 555 | CG | LEU | A | 106 | 10.653 | 84.564 | 1.905 | 1.00 | 42.45 | C |
| ATOM | 556 | CD1 | LEU | A | 106 | 10.906 | 85.341 | 3.204 | 1.00 | 40.95 | C |
| ATOM | 557 | CD2 | LEU | A | 106 | 11.993 | 84.148 | 1.293 | 1.00 | 40.34 | C |
| ATOM | 558 | C | LEU | A | 106 | 7.601 | 84.394 | 1.350 | 1.00 | 44.07 | C |
| ATOM | 559 | O | LEU | A | 106 | 7.620 | 83.960 | 0.206 | 1.00 | 44.25 | O |
| ATOM | 560 | N | LEU | A | 107 | 6.979 | 85.521 | 1.683 | 1.00 | 44.63 | N |
| ATOM | 561 | CA | LEU | A | 107 | 6.241 | 86.316 | 0.712 | 1.00 | 45.98 | C |
| ATOM | 562 | CB | LEU | A | 107 | 5.123 | 87.107 | 1.401 | 1.00 | 46.13 | C |
| ATOM | 563 | CG | LEU | A | 107 | 4.143 | 86.246 | 2.202 | 1.00 | 46.97 | C |
| ATOM | 564 | CD1 | LEU | A | 107 | 3.237 | 87.069 | 3.085 | 1.00 | 47.43 | C |
| ATOM | 565 | CD2 | LEU | A | 107 | 3.330 | 85.376 | 1.239 | 1.00 | 49.67 | C |
| ATOM | 566 | C | LEU | A | 107 | 7.145 | 87.267 | −0.066 | 1.00 | 46.90 | C |
| ATOM | 567 | O | LEU | A | 107 | 6.793 | 87.686 | −1.177 | 1.00 | 46.82 | O |
| ATOM | 568 | N | ASP | A | 108 | 8.302 | 87.601 | 0.511 | 1.00 | 47.11 | N |
| ATOM | 569 | CA | ASP | A | 108 | 9.241 | 88.537 | −0.108 | 1.00 | 47.79 | C |
| ATOM | 570 | CB | ASP | A | 108 | 8.543 | 89.875 | −0.430 | 1.00 | 47.83 | C |
| ATOM | 571 | CG | ASP | A | 108 | 9.311 | 90.725 | −1.464 | 1.00 | 50.15 | C |
| ATOM | 572 | OD1 | ASP | A | 108 | 10.299 | 90.251 | −2.085 | 1.00 | 50.90 | O |
| ATOM | 573 | OD2 | ASP | A | 108 | 8.978 | 91.903 | −1.710 | 1.00 | 52.00 | O |
| ATOM | 574 | C | ASP | A | 108 | 10.392 | 88.791 | 0.841 | 1.00 | 48.02 | C |
| ATOM | 575 | O | ASP | A | 108 | 10.319 | 88.447 | 2.025 | 1.00 | 46.97 | O |
| ATOM | 576 | N | TRP | A | 109 | 11.453 | 89.399 | 0.318 | 1.00 | 48.18 | N |
| ATOM | 577 | CA | TRP | A | 109 | 12.613 | 89.748 | 1.114 | 1.00 | 48.85 | C |
| ATOM | 578 | CB | TRP | A | 109 | 13.598 | 88.588 | 1.170 | 1.00 | 48.93 | C |
| ATOM | 579 | CG | TRP | A | 109 | 14.148 | 88.237 | −0.171 | 1.00 | 51.68 | C |
| ATOM | 580 | CD1 | TRP | A | 109 | 13.543 | 87.478 | −1.137 | 1.00 | 52.64 | C |
| ATOM | 581 | NE1 | TRP | A | 109 | 14.354 | 87.383 | −2.244 | 1.00 | 54.00 | N |
| ATOM | 582 | CE2 | TRP | A | 109 | 15.509 | 88.084 | −2.013 | 1.00 | 54.40 | C |
| ATOM | 583 | CD2 | TRP | A | 109 | 15.407 | 88.645 | −0.715 | 1.00 | 53.99 | C |
| ATOM | 584 | CE3 | TRP | A | 109 | 16.470 | 89.423 | −0.236 | 1.00 | 55.22 | C |
| ATOM | 585 | CZ3 | TRP | A | 109 | 17.577 | 89.625 | −1.056 | 1.00 | 56.91 | C |
| ATOM | 586 | CH2 | TRP | A | 109 | 17.641 | 89.061 | −2.348 | 1.00 | 57.27 | C |
| ATOM | 587 | CZ2 | TRP | A | 109 | 16.621 | 88.288 | −2.839 | 1.00 | 55.31 | C |
| ATOM | 588 | C | TRP | A | 109 | 13.302 | 90.989 | 0.555 | 1.00 | 49.39 | C |
| ATOM | 589 | O | TRP | A | 109 | 13.160 | 91.316 | −0.638 | 1.00 | 49.52 | O |
| ATOM | 590 | N | PHE | A | 110 | 14.043 | 91.672 | 1.425 | 1.00 | 49.52 | N |
| ATOM | 591 | CA | PHE | A | 110 | 14.737 | 92.912 | 1.077 | 1.00 | 50.21 | C |
| ATOM | 592 | CB | PHE | A | 110 | 14.005 | 94.130 | 1.649 | 1.00 | 49.95 | C |
| ATOM | 593 | CG | PHE | A | 110 | 12.583 | 94.259 | 1.182 | 1.00 | 51.89 | C |
| ATOM | 594 | CD1 | PHE | A | 110 | 12.268 | 95.026 | 0.061 | 1.00 | 53.85 | C |
| ATOM | 595 | CE1 | PHE | A | 110 | 10.950 | 95.139 | −0.376 | 1.00 | 54.01 | C |
| ATOM | 596 | CZ | PHE | A | 110 | 9.941 | 94.477 | 0.310 | 1.00 | 54.23 | C |
| ATOM | 597 | CE2 | PHE | A | 110 | 10.249 | 93.710 | 1.426 | 1.00 | 53.04 | C |
| ATOM | 598 | CD2 | PHE | A | 110 | 11.559 | 93.611 | 1.855 | 1.00 | 52.18 | C |
| ATOM | 599 | C | PHE | A | 110 | 16.126 | 92.848 | 1.657 | 1.00 | 50.39 | C |
| ATOM | 600 | O | PHE | A | 110 | 16.331 | 92.251 | 2.711 | 1.00 | 49.25 | O |
| ATOM | 601 | N | GLU | A | 111 | 17.087 | 93.452 | 0.966 | 1.00 | 51.10 | N |
| ATOM | 602 | CA | GLU | A | 111 | 18.440 | 93.541 | 1.494 | 1.00 | 52.22 | C |
| ATOM | 603 | CB | GLU | A | 111 | 19.450 | 93.076 | 0.457 | 1.00 | 52.56 | C |
| ATOM | 604 | CG | GLU | A | 111 | 20.896 | 93.340 | 0.835 | 1.00 | 54.50 | C |
| ATOM | 605 | CD | GLU | A | 111 | 21.857 | 92.662 | −0.109 | 1.00 | 57.61 | C |
| ATOM | 606 | OE1 | GLU | A | 111 | 21.513 | 92.561 | −1.309 | 1.00 | 60.12 | O |
| ATOM | 607 | OE2 | GLU | A | 111 | 22.937 | 92.211 | 0.348 | 1.00 | 59.23 | O |
| ATOM | 608 | C | GLU | A | 111 | 18.751 | 94.974 | 1.938 | 1.00 | 52.56 | C |
| ATOM | 609 | O | GLU | A | 111 | 18.340 | 95.943 | 1.290 | 1.00 | 53.03 | O |
| ATOM | 610 | N | ARG | A | 112 | 19.470 | 95.084 | 3.050 | 1.00 | 52.37 | N |
| ATOM | 611 | CA | ARG | A | 112 | 19.880 | 96.362 | 3.605 | 1.00 | 52.15 | C |
| ATOM | 612 | CB | ARG | A | 112 | 19.204 | 96.601 | 4.957 | 1.00 | 51.55 | C |
| ATOM | 613 | CG | ARG | A | 112 | 17.795 | 97.170 | 4.862 | 1.00 | 50.03 | C |
| ATOM | 614 | CD | ARG | A | 112 | 17.101 | 97.229 | 6.225 | 1.00 | 49.06 | C |
| ATOM | 615 | NE | ARG | A | 112 | 15.825 | 97.939 | 6.172 | 1.00 | 47.63 | N |
| ATOM | 616 | CZ | ARG | A | 112 | 15.036 | 98.135 | 7.223 | 1.00 | 48.41 | C |
| ATOM | 617 | NH1 | ARG | A | 112 | 15.379 | 97.664 | 8.420 | 1.00 | 47.11 | N |
| ATOM | 618 | NH2 | ARG | A | 112 | 13.895 | 98.797 | 7.078 | 1.00 | 48.44 | N |
| ATOM | 619 | C | ARG | A | 112 | 21.380 | 96.312 | 3.789 | 1.00 | 52.90 | C |
| ATOM | 620 | O | ARG | A | 112 | 21.972 | 95.227 | 3.727 | 1.00 | 52.95 | O |
| ATOM | 621 | N | PRO | A | 113 | 22.008 | 97.466 | 4.027 | 1.00 | 53.80 | N |
| ATOM | 622 | CA | PRO | A | 113 | 23.463 | 97.519 | 4.222 | 1.00 | 53.98 | C |
| ATOM | 623 | CB | PRO | A | 113 | 23.696 | 98.958 | 4.718 | 1.00 | 54.67 | C |
| ATOM | 624 | CG | PRO | A | 113 | 22.595 | 99.746 | 4.065 | 1.00 | 54.14 | C |
| ATOM | 625 | CD | PRO | A | 113 | 21.396 | 98.811 | 4.112 | 1.00 | 54.32 | C |
| ATOM | 626 | C | PRO | A | 113 | 23.998 | 96.489 | 5.220 | 1.00 | 54.07 | C |
| ATOM | 627 | O | PRO | A | 113 | 24.980 | 95.812 | 4.914 | 1.00 | 54.49 | O |
| ATOM | 628 | N | ASP | A | 114 | 23.373 | 96.342 | 6.382 | 1.00 | 54.16 | N |
| ATOM | 629 | CA | ASP | A | 114 | 23.903 | 95.378 | 7.346 | 1.00 | 54.11 | C |
| ATOM | 630 | CB | ASP | A | 114 | 24.430 | 96.112 | 8.575 | 1.00 | 55.71 | C |
| ATOM | 631 | CG | ASP | A | 114 | 25.631 | 96.989 | 8.245 | 1.00 | 58.46 | C |
| ATOM | 632 | OD1 | ASP | A | 114 | 25.423 | 98.057 | 7.607 | 1.00 | 61.78 | O |
| ATOM | 633 | OD2 | ASP | A | 114 | 26.805 | 96.681 | 8.573 | 1.00 | 60.03 | O |
| ATOM | 634 | C | ASP | A | 114 | 22.937 | 94.269 | 7.755 | 1.00 | 52.78 | C |
| ATOM | 635 | O | ASP | A | 114 | 23.188 | 93.548 | 8.727 | 1.00 | 53.03 | O |
| ATOM | 636 | N | SER | A | 115 | 21.852 | 94.111 | 6.999 | 1.00 | 50.98 | N |
| ATOM | 637 | CA | SER | A | 115 | 20.856 | 93.105 | 7.331 | 1.00 | 48.41 | C |
| ATOM | 638 | CB | SER | A | 115 | 19.960 | 93.649 | 8.439 | 1.00 | 48.04 | C |
| ATOM | 639 | OG | SER | A | 115 | 18.997 | 94.528 | 7.893 | 1.00 | 45.84 | O |
| ATOM | 640 | C | SER | A | 115 | 19.978 | 92.666 | 6.155 | 1.00 | 47.32 | C |
| ATOM | 641 | O | SER | A | 115 | 19.987 | 93.285 | 5.096 | 1.00 | 46.92 | O |
| ATOM | 642 | N | PHE | A | 116 | 19.198 | 91.609 | 6.381 | 1.00 | 45.66 | N |
| ATOM | 643 | CA | PHE | A | 116 | 18.171 | 91.174 | 5.446 | 1.00 | 44.29 | C |
| ATOM | 644 | CB | PHE | A | 116 | 18.457 | 89.746 | 4.994 | 1.00 | 44.76 | C |
| ATOM | 645 | CG | PHE | A | 116 | 19.567 | 89.632 | 3.980 | 1.00 | 45.24 | C |
| ATOM | 646 | CD1 | PHE | A | 116 | 20.892 | 89.484 | 4.385 | 1.00 | 45.34 | C |
| ATOM | 647 | CE1 | PHE | A | 116 | 21.915 | 89.360 | 3.447 | 1.00 | 47.24 | C |
| ATOM | 648 | CZ | PHE | A | 116 | 21.614 | 89.387 | 2.077 | 1.00 | 46.18 | C |
| ATOM | 649 | CE2 | PHE | A | 116 | 20.291 | 89.531 | 1.661 | 1.00 | 48.08 | C |
| ATOM | 650 | CD2 | PHE | A | 116 | 19.275 | 89.646 | 2.615 | 1.00 | 47.94 | C |
| ATOM | 651 | C | PHE | A | 116 | 16.824 | 91.238 | 6.141 | 1.00 | 43.43 | C |
| ATOM | 652 | O | PHE | A | 116 | 16.721 | 90.945 | 7.333 | 1.00 | 42.72 | O |
| ATOM | 653 | N | VAL | A | 117 | 15.797 | 91.641 | 5.411 | 1.00 | 42.24 | N |
| ATOM | 654 | CA | VAL | A | 117 | 14.450 | 91.681 | 5.945 | 1.00 | 41.74 | C |
| ATOM | 655 | CB | VAL | A | 117 | 13.837 | 93.087 | 5.818 | 1.00 | 41.89 | C |
| ATOM | 656 | CG1 | VAL | A | 117 | 12.473 | 93.136 | 6.447 | 1.00 | 41.40 | C |
| ATOM | 657 | CG2 | VAL | A | 117 | 14.753 | 94.122 | 6.451 | 1.00 | 42.52 | C |
| ATOM | 658 | C | VAL | A | 117 | 13.578 | 90.652 | 5.209 | 1.00 | 41.80 | C |
| ATOM | 659 | O | VAL | A | 117 | 13.507 | 90.660 | 3.974 | 1.00 | 40.77 | O |
| ATOM | 660 | N | LEU | A | 118 | 12.934 | 89.765 | 5.974 | 1.00 | 41.04 | N |
| ATOM | 661 | CA | LEU | A | 118 | 12.094 | 88.693 | 5.410 | 1.00 | 40.22 | C |
| ATOM | 662 | CB | LEU | A | 118 | 12.520 | 87.337 | 5.977 | 1.00 | 39.98 | C |
| ATOM | 663 | CG | LEU | A | 118 | 13.795 | 86.695 | 5.423 | 1.00 | 40.10 | C |
| ATOM | 664 | CD1 | LEU | A | 118 | 15.014 | 87.562 | 5.619 | 1.00 | 42.56 | C |
| ATOM | 665 | CD2 | LEU | A | 118 | 14.032 | 85.325 | 6.063 | 1.00 | 39.40 | C |
| ATOM | 666 | C | LEU | A | 118 | 10.635 | 88.939 | 5.720 | 1.00 | 40.25 | C |
| ATOM | 667 | O | LEU | A | 118 | 10.275 | 89.223 | 6.861 | 1.00 | 39.35 | O |
| ATOM | 668 | N | ILE | A | 119 | 9.795 | 88.842 | 4.700 | 1.00 | 39.45 | N |
| ATOM | 669 | CA | ILE | A | 119 | 8.370 | 89.028 | 4.876 | 1.00 | 40.37 | C |
| ATOM | 670 | CB | ILE | A | 119 | 7.774 | 89.921 | 3.756 | 1.00 | 40.19 | C |
| ATOM | 671 | CG1 | ILE | A | 119 | 8.555 | 91.248 | 3.612 | 1.00 | 41.49 | C |
| ATOM | 672 | CD1 | ILE | A | 119 | 8.540 | 92.131 | 4.855 | 1.00 | 40.16 | C |
| ATOM | 673 | CG2 | ILE | A | 119 | 6.296 | 90.136 | 3.989 | 1.00 | 39.40 | C |
| ATOM | 674 | C | ILE | A | 119 | 7.748 | 87.638 | 4.823 | 1.00 | 41.09 | C |
| ATOM | 675 | O | ILE | A | 119 | 7.793 | 86.966 | 3.788 | 1.00 | 40.59 | O |
| ATOM | 676 | N | LEU | A | 120 | 7.167 | 87.222 | 5.939 | 1.00 | 41.43 | N |
| ATOM | 677 | CA | LEU | A | 120 | 6.634 | 85.872 | 6.076 | 1.00 | 42.78 | C |
| ATOM | 678 | CB | LEU | A | 120 | 7.355 | 85.144 | 7.216 | 1.00 | 41.61 | C |
| ATOM | 679 | CG | LEU | A | 120 | 8.868 | 85.010 | 7.046 | 1.00 | 40.99 | C |
| ATOM | 680 | CD1 | LEU | A | 120 | 9.558 | 84.928 | 8.402 | 1.00 | 43.47 | C |
| ATOM | 681 | CD2 | LEU | A | 120 | 9.234 | 83.785 | 6.187 | 1.00 | 42.48 | C |
| ATOM | 682 | C | LEU | A | 120 | 5.138 | 85.922 | 6.330 | 1.00 | 44.08 | C |
| ATOM | 683 | O | LEU | A | 120 | 4.604 | 86.963 | 6.715 | 1.00 | 44.77 | O |
| ATOM | 684 | N | GLU | A | 121 | 4.449 | 84.808 | 6.109 | 1.00 | 45.28 | N |
| ATOM | 685 | CA | GLU | A | 121 | 3.026 | 84.738 | 6.436 | 1.00 | 47.09 | C |
| ATOM | 686 | CB | GLU | A | 121 | 2.430 | 83.409 | 5.985 | 1.00 | 47.99 | C |
| ATOM | 687 | CG | GLU | A | 121 | 2.534 | 83.123 | 4.497 | 1.00 | 49.97 | C |
| ATOM | 688 | CD | GLU | A | 121 | 1.959 | 81.759 | 4.170 | 1.00 | 53.32 | C |
| ATOM | 689 | OE1 | GLU | A | 121 | 0.911 | 81.714 | 3.506 | 1.00 | 55.61 | O |
| ATOM | 690 | OE2 | GLU | A | 121 | 2.548 | 80.735 | 4.586 | 1.00 | 52.97 | O |
| ATOM | 691 | C | GLU | A | 121 | 2.841 | 84.862 | 7.938 | 1.00 | 47.78 | C |
| ATOM | 692 | O | GLU | A | 121 | 3.753 | 84.550 | 8.711 | 1.00 | 47.35 | O |
| ATOM | 693 | N | ARG | A | 122 | 1.670 | 85.326 | 8.351 | 1.00 | 48.92 | N |
| ATOM | 694 | CA | ARG | A | 122 | 1.369 | 85.439 | 9.766 | 1.00 | 50.98 | C |
| ATOM | 695 | CB | ARG | A | 122 | 1.555 | 86.883 | 10.257 | 1.00 | 50.78 | C |
| ATOM | 696 | CG | ARG | A | 122 | 1.196 | 87.085 | 11.730 | 1.00 | 51.57 | C |
| ATOM | 697 | CD | ARG | A | 122 | 1.716 | 88.383 | 12.349 | 1.00 | 51.63 | C |
| ATOM | 698 | NE | ARG | A | 122 | 1.119 | 89.578 | 11.744 | 1.00 | 52.11 | N |
| ATOM | 699 | CZ | ARG | A | 122 | −0.133 | 89.977 | 11.951 | 1.00 | 51.61 | C |
| ATOM | 700 | NH1 | ARG | A | 122 | −0.937 | 89.274 | 12.741 | 1.00 | 51.42 | N |
| ATOM | 701 | NH2 | ARG | A | 122 | −0.588 | 91.070 | 11.354 | 1.00 | 50.64 | N |
| ATOM | 702 | C | ARG | A | 122 | −0.054 | 84.965 | 10.017 | 1.00 | 52.37 | C |
| ATOM | 703 | O | ARG | A | 122 | −1.005 | 85.749 | 9.922 | 1.00 | 52.94 | O |
| ATOM | 704 | N | PRO | A | 123 | −0.211 | 83.682 | 10.328 | 1.00 | 53.57 | N |
| ATOM | 705 | CA | PRO | A | 123 | −1.529 | 83.141 | 10.672 | 1.00 | 54.00 | C |
| ATOM | 706 | CB | PRO | A | 123 | −1.227 | 81.669 | 10.973 | 1.00 | 54.39 | C |
| ATOM | 707 | CG | PRO | A | 123 | 0.057 | 81.394 | 10.250 | 1.00 | 54.18 | C |
| ATOM | 708 | CD | PRO | A | 123 | 0.845 | 82.652 | 10.398 | 1.00 | 53.87 | C |
| ATOM | 709 | C | PRO | A | 123 | −2.012 | 83.835 | 11.928 | 1.00 | 54.39 | C |
| ATOM | 710 | O | PRO | A | 123 | −1.172 | 84.333 | 12.676 | 1.00 | 54.50 | O |
| ATOM | 711 | N | GLU | A | 124 | −3.322 | 83.862 | 12.164 | 1.00 | 54.85 | N |
| ATOM | 712 | CA | GLU | A | 124 | −3.859 | 84.533 | 13.348 | 1.00 | 55.63 | C |
| ATOM | 713 | CB | GLU | A | 124 | −3.870 | 86.046 | 13.089 | 1.00 | 56.97 | C |
| ATOM | 714 | CG | GLU | A | 124 | −3.856 | 86.946 | 14.335 | 1.00 | 62.93 | C |
| ATOM | 715 | CD | GLU | A | 124 | −4.020 | 88.417 | 13.933 | 1.00 | 69.90 | C |
| ATOM | 716 | OE1 | GLU | A | 124 | −4.962 | 88.742 | 13.153 | 1.00 | 72.13 | O |
| ATOM | 717 | OE2 | GLU | A | 124 | −3.195 | 89.256 | 14.385 | 1.00 | 71.98 | O |
| ATOM | 718 | C | GLU | A | 124 | −5.270 | 84.018 | 13.671 | 1.00 | 53.97 | C |
| ATOM | 719 | O | GLU | A | 124 | −6.093 | 83.910 | 12.764 | 1.00 | 54.52 | O |
| ATOM | 720 | N | PRO | A | 125 | −5.563 | 83.673 | 14.930 | 1.00 | 52.32 | N |
| ATOM | 721 | CA | PRO | A | 125 | −4.601 | 83.673 | 16.040 | 1.00 | 51.14 | C |
| ATOM | 722 | CB | PRO | A | 125 | −5.504 | 83.553 | 17.275 | 1.00 | 51.19 | C |
| ATOM | 723 | CG | PRO | A | 125 | −6.689 | 82.766 | 16.783 | 1.00 | 50.94 | C |
| ATOM | 724 | CD | PRO | A | 125 | −6.906 | 83.255 | 15.374 | 1.00 | 51.87 | C |
| ATOM | 725 | C | PRO | A | 125 | −3.694 | 82.453 | 15.970 | 1.00 | 50.27 | C |
| ATOM | 726 | O | PRO | A | 125 | −4.057 | 81.436 | 15.379 | 1.00 | 49.98 | O |
| ATOM | 727 | N | VAL | A | 126 | −2.526 | 82.562 | 16.588 | 1.00 | 49.17 | N |
| ATOM | 728 | CA | VAL | A | 126 | −1.525 | 81.526 | 16.500 | 1.00 | 48.07 | C |
| ATOM | 729 | CB | VAL | A | 126 | −0.508 | 81.866 | 15.373 | 1.00 | 48.55 | C |
| ATOM | 730 | CG1 | VAL | A | 126 | 0.307 | 83.104 | 15.726 | 1.00 | 49.90 | C |
| ATOM | 731 | CG2 | VAL | A | 126 | 0.400 | 80.711 | 15.096 | 1.00 | 50.31 | C |
| ATOM | 732 | C | VAL | A | 126 | −0.848 | 81.346 | 17.855 | 1.00 | 46.31 | C |
| ATOM | 733 | O | VAL | A | 126 | −0.787 | 82.290 | 18.644 | 1.00 | 46.40 | O |
| ATOM | 734 | N | GLN | A | 127 | −0.360 | 80.129 | 18.117 | 1.00 | 43.89 | N |
| ATOM | 735 | CA | GLN | A | 127 | 0.495 | 79.825 | 19.270 | 1.00 | 41.91 | C |
| ATOM | 736 | CB | GLN | A | 127 | −0.356 | 79.311 | 20.438 | 1.00 | 41.96 | C |
| ATOM | 737 | CG | GLN | A | 127 | 0.414 | 79.085 | 21.751 | 1.00 | 41.25 | C |
| ATOM | 738 | CD | GLN | A | 127 | −0.498 | 78.618 | 22.880 | 1.00 | 41.99 | C |
| ATOM | 739 | OE1 | GLN | A | 127 | −1.346 | 77.744 | 22.688 | 1.00 | 41.61 | O |
| ATOM | 740 | NE2 | GLN | A | 127 | −0.336 | 79.214 | 24.052 | 1.00 | 40.70 | N |
| ATOM | 741 | C | GLN | A | 127 | 1.500 | 78.750 | 18.868 | 1.00 | 41.00 | C |
| ATOM | 742 | O | GLN | A | 127 | 1.136 | 77.807 | 18.153 | 1.00 | 40.30 | O |
| ATOM | 743 | N | ASP | A | 128 | 2.755 | 78.874 | 19.307 | 1.00 | 40.28 | N |
| ATOM | 744 | CA | ASP | A | 128 | 3.719 | 77.829 | 18.995 | 1.00 | 39.77 | C |
| ATOM | 745 | CB | ASP | A | 128 | 5.174 | 78.319 | 19.018 | 1.00 | 40.70 | C |
| ATOM | 746 | CG | ASP | A | 128 | 5.670 | 78.691 | 20.390 | 1.00 | 42.88 | C |
| ATOM | 747 | OD1 | ASP | A | 128 | 5.553 | 77.903 | 21.369 | 1.00 | 46.75 | O |
| ATOM | 748 | OD2 | ASP | A | 128 | 6.231 | 79.788 | 20.562 | 1.00 | 48.33 | O |
| ATOM | 749 | C | ASP | A | 128 | 3.482 | 76.609 | 19.881 | 1.00 | 39.44 | C |
| ATOM | 750 | O | ASP | A | 128 | 2.898 | 76.723 | 20.978 | 1.00 | 38.51 | O |
| ATOM | 751 | N | LEU | A | 129 | 3.908 | 75.443 | 19.392 | 1.00 | 37.35 | N |
| ATOM | 752 | CA | LEU | A | 129 | 3.665 | 74.196 | 20.084 | 1.00 | 35.60 | C |
| ATOM | 753 | CB | LEU | A | 129 | 4.146 | 73.015 | 19.224 | 1.00 | 33.96 | C |
| ATOM | 754 | CG | LEU | A | 129 | 3.950 | 71.607 | 19.773 | 1.00 | 34.42 | C |
| ATOM | 755 | CD1 | LEU | A | 129 | 2.485 | 71.352 | 20.108 | 1.00 | 29.98 | C |
| ATOM | 756 | CD2 | LEU | A | 129 | 4.490 | 70.573 | 18.768 | 1.00 | 33.34 | C |
| ATOM | 757 | C | LEU | A | 129 | 4.281 | 74.165 | 21.489 | 1.00 | 35.69 | C |
| ATOM | 758 | O | LEU | A | 129 | 3.730 | 73.565 | 22.403 | 1.00 | 34.94 | O |
| ATOM | 759 | N | PHE | A | 130 | 5.422 | 74.804 | 21.659 | 1.00 | 36.83 | N |
| ATOM | 760 | CA | PHE | A | 130 | 6.047 | 74.850 | 22.976 | 1.00 | 38.88 | C |
| ATOM | 761 | CB | PHE | A | 130 | 7.342 | 75.665 | 22.930 | 1.00 | 39.30 | C |
| ATOM | 762 | CG | PHE | A | 130 | 8.070 | 75.714 | 24.254 | 1.00 | 42.49 | C |
| ATOM | 763 | CD1 | PHE | A | 130 | 7.678 | 76.621 | 25.251 | 1.00 | 45.52 | C |
| ATOM | 764 | CE1 | PHE | A | 130 | 8.349 | 76.680 | 26.489 | 1.00 | 46.12 | C |
| ATOM | 765 | CZ | PHE | A | 130 | 9.404 | 75.807 | 26.747 | 1.00 | 46.89 | C |
| ATOM | 766 | CE2 | PHE | A | 130 | 9.806 | 74.886 | 25.758 | 1.00 | 47.58 | C |
| ATOM | 767 | CD2 | PHE | A | 130 | 9.132 | 74.851 | 24.514 | 1.00 | 44.28 | C |
| ATOM | 768 | C | PHE | A | 130 | 5.099 | 75.492 | 23.989 | 1.00 | 39.51 | C |
| ATOM | 769 | O | PHE | A | 130 | 4.849 | 74.931 | 25.064 | 1.00 | 38.80 | O |
| ATOM | 770 | N | ASP | A | 131 | 4.596 | 76.679 | 23.657 | 1.00 | 40.69 | N |
| ATOM | 771 | CA | ASP | A | 131 | 3.719 | 77.401 | 24.583 | 1.00 | 42.23 | C |
| ATOM | 772 | CB | ASP | A | 131 | 3.418 | 78.805 | 24.088 | 1.00 | 43.13 | C |
| ATOM | 773 | CG | ASP | A | 131 | 4.620 | 79.699 | 24.113 | 1.00 | 44.46 | C |
| ATOM | 774 | OD1 | ASP | A | 131 | 5.574 | 79.431 | 24.874 | 1.00 | 47.95 | O |
| ATOM | 775 | OD2 | ASP | A | 131 | 4.702 | 80.700 | 23.375 | 1.00 | 49.81 | O |
| ATOM | 776 | C | ASP | A | 131 | 2.433 | 76.642 | 24.771 | 1.00 | 42.09 | C |
| ATOM | 111 | O | ASP | A | 131 | 1.888 | 76.600 | 25.877 | 1.00 | 42.20 | O |
| ATOM | 778 | N | PHE | A | 132 | 1.972 | 76.001 | 23.696 | 1.00 | 41.67 | N |
| ATOM | 779 | CA | PHE | A | 132 | 0.760 | 75.195 | 23.744 | 1.00 | 41.50 | C |
| ATOM | 780 | CB | PHE | A | 132 | 0.459 | 74.630 | 22.358 | 1.00 | 41.82 | C |
| ATOM | 781 | CG | PHE | A | 132 | −0.854 | 73.909 | 22.263 | 1.00 | 40.63 | C |
| ATOM | 782 | CD1 | PHE | A | 132 | −2.039 | 74.618 | 22.148 | 1.00 | 40.87 | C |
| ATOM | 783 | CE1 | PHE | A | 132 | −3.251 | 73.965 | 22.053 | 1.00 | 41.37 | C |
| ATOM | 784 | CZ | PHE | A | 132 | −3.297 | 72.581 | 22.062 | 1.00 | 41.96 | C |
| ATOM | 785 | CE2 | PHE | A | 132 | −2.123 | 71.855 | 22.173 | 1.00 | 40.31 | C |
| ATOM | 786 | CD2 | PHE | A | 132 | −0.910 | 72.524 | 22.281 | 1.00 | 42.21 | C |
| ATOM | 787 | C | PHE | A | 132 | 0.902 | 74.058 | 24.760 | 1.00 | 42.73 | C |
| ATOM | 788 | O | PHE | A | 132 | −0.006 | 73.809 | 25.570 | 1.00 | 42.57 | O |
| ATOM | 789 | N | ILE | A | 133 | 2.040 | 73.369 | 24.718 | 1.00 | 42.81 | N |
| ATOM | 790 | CA | ILE | A | 133 | 2.286 | 72.247 | 25.630 | 1.00 | 43.91 | C |
| ATOM | 791 | CB | ILE | A | 133 | 3.487 | 71.395 | 25.138 | 1.00 | 42.90 | C |
| ATOM | 792 | CG1 | ILE | A | 133 | 3.071 | 70.570 | 23.920 | 1.00 | 41.19 | C |
| ATOM | 793 | CD1 | ILE | A | 133 | 4.220 | 69.961 | 23.174 | 1.00 | 41.52 | C |
| ATOM | 794 | CG2 | ILE | A | 133 | 4.026 | 70.461 | 26.249 | 1.00 | 42.29 | C |
| ATOM | 795 | C | ILE | A | 133 | 2.514 | 72.780 | 27.054 | 1.00 | 46.10 | C |
| ATOM | 796 | O | ILE | A | 133 | 2.046 | 72.191 | 28.023 | 1.00 | 46.11 | O |
| ATOM | 797 | N | THR | A | 134 | 3.231 | 73.894 | 27.162 | 1.00 | 48.42 | N |
| ATOM | 798 | CA | THR | A | 134 | 3.487 | 74.529 | 28.453 | 1.00 | 51.23 | C |
| ATOM | 799 | CB | THR | A | 134 | 4.314 | 75.805 | 28.261 | 1.00 | 51.04 | C |
| ATOM | 800 | OG1 | THR | A | 134 | 5.695 | 75.440 | 28.115 | 1.00 | 52.55 | O |
| ATOM | 801 | CG2 | THR | A | 134 | 4.293 | 76.695 | 29.524 | 1.00 | 53.00 | C |
| ATOM | 802 | C | THR | A | 134 | 2.179 | 74.851 | 29.159 | 1.00 | 52.34 | C |
| ATOM | 803 | O | THR | A | 134 | 2.069 | 74.673 | 30.365 | 1.00 | 53.35 | O |
| ATOM | 804 | N | GLU | A | 135 | 1.188 | 75.303 | 28.400 | 1.00 | 53.42 | N |
| ATOM | 805 | CA | GLU | A | 135 | −0.110 | 75.662 | 28.959 | 1.00 | 54.43 | C |
| ATOM | 806 | CB | GLU | A | 135 | −0.840 | 76.644 | 28.038 | 1.00 | 55.03 | C |
| ATOM | 807 | CG | GLU | A | 135 | −0.137 | 78.004 | 27.941 | 1.00 | 59.01 | C |
| ATOM | 808 | CD | GLU | A | 135 | −0.981 | 79.054 | 27.234 | 1.00 | 62.58 | C |
| ATOM | 809 | OE1 | GLU | A | 135 | −1.942 | 78.685 | 26.505 | 1.00 | 64.20 | O |
| ATOM | 810 | OE2 | GLU | A | 135 | −0.675 | 80.254 | 27.412 | 1.00 | 63.63 | O |
| ATOM | 811 | C | GLU | A | 135 | −1.009 | 74.468 | 29.215 | 1.00 | 53.96 | C |
| ATOM | 812 | O | GLU | A | 135 | −1.743 | 74.440 | 30.206 | 1.00 | 54.61 | O |
| ATOM | 813 | N | ARG | A | 136 | −0.975 | 73.490 | 28.318 | 1.00 | 52.83 | N |
| ATOM | 814 | CA | ARG | A | 136 | −1.947 | 72.404 | 28.385 | 1.00 | 51.61 | C |
| ATOM | 815 | CB | ARG | A | 136 | −2.646 | 72.261 | 27.036 | 1.00 | 52.42 | C |
| ATOM | 816 | CG | ARG | A | 136 | −3.486 | 73.503 | 26.736 | 1.00 | 55.25 | C |
| ATOM | 817 | CD | ARG | A | 136 | −4.130 | 73.538 | 25.378 | 1.00 | 58.99 | C |
| ATOM | 818 | NE | ARG | A | 136 | −4.990 | 72.381 | 25.145 | 1.00 | 60.79 | N |
| ATOM | 819 | CZ | ARG | A | 136 | −6.072 | 72.415 | 24.379 | 1.00 | 61.01 | C |
| ATOM | 820 | NH1 | ARG | A | 136 | −6.425 | 73.559 | 23.777 | 1.00 | 60.57 | N |
| ATOM | 821 | NH2 | ARG | A | 136 | −6.793 | 71.310 | 24.207 | 1.00 | 60.45 | N |
| ATOM | 822 | C | ARG | A | 136 | −1.376 | 71.086 | 28.887 | 1.00 | 50.04 | C |
| ATOM | 823 | O | ARG | A | 136 | −2.116 | 70.144 | 29.119 | 1.00 | 50.37 | O |
| ATOM | 824 | N | GLY | A | 137 | −0.062 | 71.033 | 29.081 | 1.00 | 48.53 | N |
| ATOM | 825 | CA | GLY | A | 137 | 0.597 | 69.799 | 29.477 | 1.00 | 46.78 | C |
| ATOM | 826 | C | GLY | A | 137 | 0.532 | 68.744 | 28.381 | 1.00 | 44.89 | C |
| ATOM | 827 | O | GLY | A | 137 | 0.183 | 69.046 | 27.232 | 1.00 | 45.03 | O |
| ATOM | 828 | N | ALA | A | 138 | 0.849 | 67.509 | 28.748 | 1.00 | 43.07 | N |
| ATOM | 829 | CA | ALA | A | 138 | 0.841 | 66.374 | 27.833 | 1.00 | 41.43 | C |
| ATOM | 830 | CB | ALA | A | 138 | 1.023 | 65.083 | 28.602 | 1.00 | 41.46 | C |
| ATOM | 831 | C | ALA | A | 138 | −0.433 | 66.321 | 26.990 | 1.00 | 40.58 | C |
| ATOM | 832 | O | ALA | A | 138 | −1.533 | 66.476 | 27.491 | 1.00 | 40.85 | O |
| ATOM | 833 | N | LEU | A | 139 | −0.274 | 66.108 | 25.693 | 1.00 | 38.87 | N |
| ATOM | 834 | CA | LEU | A | 139 | −1.415 | 66.107 | 24.794 | 1.00 | 37.16 | C |
| ATOM | 835 | CB | LEU | A | 139 | −0.994 | 66.557 | 23.392 | 1.00 | 34.91 | C |
| ATOM | 836 | CG | LEU | A | 139 | −0.224 | 67.881 | 23.369 | 1.00 | 36.75 | C |
| ATOM | 837 | CD1 | LEU | A | 139 | 0.082 | 68.270 | 21.920 | 1.00 | 35.15 | C |
| ATOM | 838 | CD2 | LEU | A | 139 | −1.002 | 68.999 | 24.124 | 1.00 | 35.61 | C |
| ATOM | 839 | C | LEU | A | 139 | −2.039 | 64.741 | 24.731 | 1.00 | 36.86 | C |
| ATOM | 840 | O | LEU | A | 139 | −1.338 | 63.733 | 24.761 | 1.00 | 37.14 | O |
| ATOM | 841 | N | GLN | A | 140 | −3.362 | 64.714 | 24.626 | 1.00 | 37.15 | N |
| ATOM | 842 | CA | GLN | A | 140 | −4.071 | 63.473 | 24.348 | 1.00 | 38.86 | C |
| ATOM | 843 | CB | GLN | A | 140 | −5.566 | 63.726 | 24.208 | 1.00 | 39.38 | C |
| ATOM | 844 | CG | GLN | A | 140 | −6.266 | 63.885 | 25.540 | 1.00 | 45.52 | C |
| ATOM | 845 | CD | GLN | A | 140 | −7.649 | 64.493 | 25.395 | 1.00 | 52.38 | C |
| ATOM | 846 | OE1 | GLN | A | 140 | −8.442 | 64.070 | 24.534 | 1.00 | 54.06 | O |
| ATOM | 847 | NE2 | GLN | A | 140 | −7.949 | 65.488 | 26.234 | 1.00 | 55.44 | N |
| ATOM | 848 | C | GLN | A | 140 | −3.532 | 62.890 | 23.062 | 1.00 | 37.84 | C |
| ATOM | 849 | O | GLN | A | 140 | −3.192 | 63.643 | 22.141 | 1.00 | 37.63 | O |
| ATOM | 850 | N | GLU | A | 141 | −3.449 | 61.559 | 22.996 | 1.00 | 37.43 | N |
| ATOM | 851 | CA | GLU | A | 141 | −2.897 | 60.881 | 21.808 | 1.00 | 37.42 | C |
| ATOM | 852 | CB | GLU | A | 141 | −2.849 | 59.373 | 22.030 | 1.00 | 37.84 | C |
| ATOM | 853 | CG | GLU | A | 141 | −1.883 | 59.033 | 23.164 | 1.00 | 38.10 | C |
| ATOM | 854 | CD | GLU | A | 141 | −1.571 | 57.568 | 23.263 | 1.00 | 36.74 | C |
| ATOM | 855 | OE1 | GLU | A | 141 | −1.639 | 56.867 | 22.233 | 1.00 | 35.36 | O |
| ATOM | 856 | OE2 | GLU | A | 141 | −1.261 | 57.117 | 24.383 | 1.00 | 37.15 | O |
| ATOM | 857 | C | GLU | A | 141 | −3.596 | 61.227 | 20.498 | 1.00 | 36.95 | C |
| ATOM | 858 | O | GLU | A | 141 | −2.958 | 61.254 | 19.443 | 1.00 | 36.70 | O |
| ATOM | 859 | N | GLU | A | 142 | −4.900 | 61.497 | 20.566 | 1.00 | 36.62 | N |
| ATOM | 860 | CA | GLU | A | 142 | −5.654 | 61.865 | 19.373 | 1.00 | 36.84 | C |
| ATOM | 861 | CB | GLU | A | 142 | −7.151 | 62.019 | 19.677 | 1.00 | 37.69 | C |
| ATOM | 862 | CG | GLU | A | 142 | −7.957 | 62.396 | 18.443 | 1.00 | 39.42 | C |
| ATOM | 863 | CD | GLU | A | 142 | −9.440 | 62.567 | 18.730 | 1.00 | 43.91 | C |
| ATOM | 864 | OE1 | GLU | A | 142 | −9.809 | 63.542 | 19.421 | 1.00 | 44.11 | O |
| ATOM | 865 | OE2 | GLU | A | 142 | −10.233 | 61.727 | 18.254 | 1.00 | 45.16 | O |
| ATOM | 866 | C | GLU | A | 142 | −5.127 | 63.181 | 18.814 | 1.00 | 35.87 | C |
| ATOM | 867 | O | GLU | A | 142 | −4.975 | 63.336 | 17.601 | 1.00 | 35.78 | O |
| ATOM | 868 | N | LEU | A | 143 | −4.857 | 64.120 | 19.709 | 1.00 | 34.68 | N |
| ATOM | 869 | CA | LEU | A | 143 | −4.343 | 65.422 | 19.333 | 1.00 | 33.92 | C |
| ATOM | 870 | CB | LEU | A | 143 | −4.434 | 66.378 | 20.526 | 1.00 | 33.86 | C |
| ATOM | 871 | CG | LEU | A | 143 | −3.933 | 67.812 | 20.341 | 1.00 | 33.72 | C |
| ATOM | 872 | CD1 | LEU | A | 143 | −4.656 | 68.402 | 19.137 | 1.00 | 31.14 | C |
| ATOM | 873 | CD2 | LEU | A | 143 | −4.227 | 68.624 | 21.591 | 1.00 | 34.84 | C |
| ATOM | 874 | C | LEU | A | 143 | −2.898 | 65.304 | 18.842 | 1.00 | 34.15 | C |
| ATOM | 875 | O | LEU | A | 143 | −2.559 | 65.834 | 17.786 | 1.00 | 34.53 | O |
| ATOM | 876 | N | ALA | A | 144 | −2.060 | 64.586 | 19.596 | 1.00 | 33.23 | N |
| ATOM | 877 | CA | ALA | A | 144 | −0.669 | 64.366 | 19.204 | 1.00 | 32.59 | C |
| ATOM | 878 | CB | ALA | A | 144 | 0.046 | 63.540 | 20.247 | 1.00 | 32.52 | C |
| ATOM | 879 | C | ALA | A | 144 | −0.598 | 63.676 | 17.844 | 1.00 | 32.09 | C |
| ATOM | 880 | O | ALA | A | 144 | 0.240 | 64.019 | 17.038 | 1.00 | 31.77 | O |
| ATOM | 881 | N | ARG | A | 145 | −1.494 | 62.720 | 17.587 | 1.00 | 32.32 | N |
| ATOM | 882 | CA | ARG | A | 145 | −1.545 | 62.050 | 16.293 | 1.00 | 32.91 | C |
| ATOM | 883 | CB | ARG | A | 145 | −2.600 | 60.939 | 16.295 | 1.00 | 33.17 | C |
| ATOM | 884 | CG | ARG | A | 145 | −2.769 | 60.208 | 14.961 | 1.00 | 35.72 | C |
| ATOM | 885 | CD | ARG | A | 145 | −3.871 | 59.129 | 14.976 | 1.00 | 37.67 | C |
| ATOM | 886 | NE | ARG | A | 145 | −3.583 | 58.127 | 15.993 | 1.00 | 39.30 | N |
| ATOM | 887 | CZ | ARG | A | 145 | −4.264 | 57.978 | 17.127 | 1.00 | 41.07 | C |
| ATOM | 888 | NH1 | ARG | A | 145 | −5.331 | 58.736 | 17.399 | 1.00 | 41.09 | N |
| ATOM | 889 | NH2 | ARG | A | 145 | −3.884 | 57.050 | 17.987 | 1.00 | 40.07 | N |
| ATOM | 890 | C | ARG | A | 145 | −1.789 | 63.044 | 15.155 | 1.00 | 33.07 | C |
| ATOM | 891 | O | ARG | A | 145 | −1.063 | 63.050 | 14.158 | 1.00 | 32.72 | O |
| ATOM | 892 | N | SER | A | 146 | −2.797 | 63.897 | 15.310 | 1.00 | 33.62 | N |
| ATOM | 893 | CA | SER | A | 146 | −3.103 | 64.896 | 14.287 | 1.00 | 33.98 | C |
| ATOM | 894 | CB | SER | A | 146 | −4.332 | 65.711 | 14.700 | 1.00 | 34.87 | C |
| ATOM | 895 | OG | SER | A | 146 | −4.556 | 66.758 | 13.767 | 1.00 | 37.55 | O |
| ATOM | 896 | C | SER | A | 146 | −1.920 | 65.837 | 14.058 | 1.00 | 34.16 | C |
| ATOM | 897 | O | SER | A | 146 | −1.518 | 66.064 | 12.917 | 1.00 | 34.41 | O |
| ATOM | 898 | N | PHE | A | 147 | −1.349 | 66.347 | 15.154 | 1.00 | 32.41 | N |
| ATOM | 899 | CA | PHE | A | 147 | −0.235 | 67.278 | 15.088 | 1.00 | 32.56 | C |
| ATOM | 900 | CB | PHE | A | 147 | 0.117 | 67.793 | 16.481 | 1.00 | 32.83 | C |
| ATOM | 901 | CG | PHE | A | 147 | −0.765 | 68.925 | 16.972 | 1.00 | 34.64 | C |
| ATOM | 902 | CD1 | PHE | A | 147 | −1.916 | 69.303 | 16.275 | 1.00 | 34.72 | C |
| ATOM | 903 | CE1 | PHE | A | 147 | −2.725 | 70.330 | 16.744 | 1.00 | 37.91 | C |
| ATOM | 904 | CZ | PHE | A | 147 | −2.380 | 71.009 | 17.911 | 1.00 | 36.29 | C |
| ATOM | 905 | CE2 | PHE | A | 147 | −1.223 | 70.642 | 18.617 | 1.00 | 36.31 | C |
| ATOM | 906 | CD2 | PHE | A | 147 | −0.430 | 69.603 | 18.143 | 1.00 | 34.51 | C |
| ATOM | 907 | C | PHE | A | 147 | 1.005 | 66.617 | 14.491 | 1.00 | 32.09 | C |
| ATOM | 908 | O | PHE | A | 147 | 1.647 | 67.190 | 13.625 | 1.00 | 31.07 | O |
| ATOM | 909 | N | PHE | A | 148 | 1.357 | 65.436 | 14.992 | 1.00 | 32.05 | N |
| ATOM | 910 | CA | PHE | A | 148 | 2.516 | 64.706 | 14.486 | 1.00 | 32.29 | C |
| ATOM | 911 | CB | PHE | A | 148 | 2.701 | 63.391 | 15.247 | 1.00 | 32.29 | C |
| ATOM | 912 | CG | PHE | A | 148 | 4.061 | 62.783 | 15.070 | 1.00 | 32.26 | C |
| ATOM | 913 | CD1 | PHE | A | 148 | 5.212 | 63.527 | 15.349 | 1.00 | 32.46 | C |
| ATOM | 914 | CE1 | PHE | A | 148 | 6.477 | 62.979 | 15.206 | 1.00 | 29.15 | C |
| ATOM | 915 | CZ | PHE | A | 148 | 6.610 | 61.665 | 14.771 | 1.00 | 30.20 | C |
| ATOM | 916 | CE2 | PHE | A | 148 | 5.465 | 60.909 | 14.475 | 1.00 | 30.97 | C |
| ATOM | 917 | CD2 | PHE | A | 148 | 4.198 | 61.469 | 14.634 | 1.00 | 31.93 | C |
| ATOM | 918 | C | PHE | A | 148 | 2.385 | 64.405 | 12.990 | 1.00 | 32.02 | C |
| ATOM | 919 | O | PHE | A | 148 | 3.343 | 64.536 | 12.238 | 1.00 | 32.46 | O |
| ATOM | 920 | N | TRP | A | 149 | 1.196 | 64.006 | 12.571 | 1.00 | 31.64 | N |
| ATOM | 921 | CA | TRP | A | 149 | 0.960 | 63.687 | 11.169 | 1.00 | 32.22 | C |
| ATOM | 922 | CB | TRP | A | 149 | −0.469 | 63.221 | 10.973 | 1.00 | 32.32 | C |
| ATOM | 923 | CG | TRP | A | 149 | −0.814 | 62.851 | 9.562 | 1.00 | 33.58 | C |
| ATOM | 924 | CD1 | TRP | A | 149 | −1.276 | 63.695 | 8.583 | 1.00 | 35.97 | C |
| ATOM | 925 | NE1 | TRP | A | 149 | −1.497 | 62.992 | 7.422 | 1.00 | 38.04 | N |
| ATOM | 926 | CE2 | TRP | A | 149 | −1.201 | 61.670 | 7.635 | 1.00 | 35.85 | C |
| ATOM | 927 | CD2 | TRP | A | 149 | −0.773 | 61.542 | 8.978 | 1.00 | 32.84 | C |
| ATOM | 928 | CE3 | TRP | A | 149 | −0.396 | 60.273 | 9.445 | 1.00 | 32.94 | C |
| ATOM | 929 | CZ3 | TRP | A | 149 | −0.480 | 59.181 | 8.574 | 1.00 | 32.70 | C |
| ATOM | 930 | CH2 | TRP | A | 149 | −0.914 | 59.352 | 7.244 | 1.00 | 35.78 | C |
| ATOM | 931 | CZ2 | TRP | A | 149 | −1.279 | 60.584 | 6.763 | 1.00 | 35.70 | C |
| ATOM | 932 | C | TRP | A | 149 | 1.228 | 64.908 | 10.309 | 1.00 | 32.05 | C |
| ATOM | 933 | O | TRP | A | 149 | 1.926 | 64.810 | 9.309 | 1.00 | 31.43 | O |
| ATOM | 934 | N | GLN | A | 150 | 0.720 | 66.078 | 10.729 | 1.00 | 31.85 | N |
| ATOM | 935 | CA | GLN | A | 150 | 0.948 | 67.309 | 9.966 | 1.00 | 31.22 | C |
| ATOM | 936 | CB | GLN | A | 150 | 0.132 | 68.489 | 10.527 | 1.00 | 30.76 | C |
| ATOM | 937 | CG | GLN | A | 150 | −1.376 | 68.335 | 10.336 | 1.00 | 32.09 | C |
| ATOM | 938 | CD | GLN | A | 150 | −2.126 | 69.553 | 10.773 | 1.00 | 34.62 | C |
| ATOM | 939 | OE1 | GLN | A | 150 | −1.850 | 70.656 | 10.292 | 1.00 | 34.95 | O |
| ATOM | 940 | NE2 | GLN | A | 150 | −3.064 | 69.376 | 11.704 | 1.00 | 35.33 | N |
| ATOM | 941 | C | GLN | A | 150 | 2.414 | 67.686 | 9.932 | 1.00 | 31.38 | C |
| ATOM | 942 | O | GLN | A | 150 | 2.884 | 68.278 | 8.942 | 1.00 | 31.25 | O |
| ATOM | 943 | N | VAL | A | 151 | 3.143 | 67.400 | 11.014 | 1.00 | 30.59 | N |
| ATOM | 944 | CA | VAL | A | 151 | 4.576 | 67.691 | 11.006 | 1.00 | 31.25 | C |
| ATOM | 945 | CB | VAL | A | 151 | 5.222 | 67.562 | 12.407 | 1.00 | 31.59 | C |
| ATOM | 946 | CG1 | VAL | A | 151 | 6.736 | 67.703 | 12.328 | 1.00 | 33.21 | C |
| ATOM | 947 | CG2 | VAL | A | 151 | 4.661 | 68.652 | 13.325 | 1.00 | 31.32 | C |
| ATOM | 948 | C | VAL | A | 151 | 5.259 | 66.780 | 9.981 | 1.00 | 30.80 | C |
| ATOM | 949 | O | VAL | A | 151 | 6.140 | 67.215 | 9.239 | 1.00 | 30.84 | O |
| ATOM | 950 | N | LEU | A | 152 | 4.842 | 65.521 | 9.939 | 1.00 | 31.70 | N |
| ATOM | 951 | CA | LEU | A | 152 | 5.429 | 64.564 | 9.000 | 1.00 | 32.14 | C |
| ATOM | 952 | CB | LEU | A | 152 | 4.792 | 63.179 | 9.194 | 1.00 | 32.39 | C |
| ATOM | 953 | CG | LEU | A | 152 | 5.513 | 62.176 | 10.123 | 1.00 | 33.85 | C |
| ATOM | 954 | CD1 | LEU | A | 152 | 6.723 | 61.611 | 9.411 | 1.00 | 35.80 | C |
| ATOM | 955 | CD2 | LEU | A | 152 | 5.950 | 62.799 | 11.422 | 1.00 | 36.98 | C |
| ATOM | 956 | C | LEU | A | 152 | 5.215 | 65.052 | 7.567 | 1.00 | 31.63 | C |
| ATOM | 957 | O | LEU | A | 152 | 6.131 | 65.024 | 6.769 | 1.00 | 32.44 | O |
| ATOM | 958 | N | GLU | A | 153 | 3.997 | 65.471 | 7.252 | 1.00 | 31.63 | N |
| ATOM | 959 | CA | GLU | A | 153 | 3.671 | 65.980 | 5.907 | 1.00 | 32.26 | C |
| ATOM | 960 | CB | GLU | A | 153 | 2.177 | 66.330 | 5.780 | 1.00 | 32.59 | C |
| ATOM | 961 | CG | GLU | A | 153 | 1.233 | 65.126 | 5.736 | 1.00 | 33.60 | C |
| ATOM | 962 | CD | GLU | A | 153 | 1.423 | 64.215 | 4.510 | 1.00 | 35.47 | C |
| ATOM | 963 | OE1 | GLU | A | 153 | 1.617 | 64.717 | 3.392 | 1.00 | 38.01 | O |
| ATOM | 964 | OE2 | GLU | A | 153 | 1.380 | 62.991 | 4.659 | 1.00 | 34.71 | O |
| ATOM | 965 | C | GLU | A | 153 | 4.538 | 67.191 | 5.562 | 1.00 | 31.87 | C |
| ATOM | 966 | O | GLU | A | 153 | 5.076 | 67.281 | 4.449 | 1.00 | 30.83 | O |
| ATOM | 967 | N | ALA | A | 154 | 4.716 | 68.101 | 6.531 | 1.00 | 31.04 | N |
| ATOM | 968 | CA | ALA | A | 154 | 5.548 | 69.291 | 6.318 | 1.00 | 30.48 | C |
| ATOM | 969 | CB | ALA | A | 154 | 5.440 | 70.278 | 7.537 | 1.00 | 31.18 | C |
| ATOM | 970 | C | ALA | A | 154 | 7.002 | 68.933 | 6.082 | 1.00 | 31.22 | C |
| ATOM | 971 | O | ALA | A | 154 | 7.683 | 69.544 | 5.238 | 1.00 | 30.96 | O |
| ATOM | 972 | N | VAL | A | 155 | 7.504 | 67.967 | 6.842 | 1.00 | 31.29 | N |
| ATOM | 973 | CA | VAL | A | 155 | 8.898 | 67.580 | 6.704 | 1.00 | 32.75 | C |
| ATOM | 974 | CB | VAL | A | 155 | 9.335 | 66.651 | 7.856 | 1.00 | 32.61 | C |
| ATOM | 975 | CG1 | VAL | A | 155 | 10.729 | 66.132 | 7.631 | 1.00 | 33.47 | C |
| ATOM | 976 | CG2 | VAL | A | 155 | 9.292 | 67.439 | 9.189 | 1.00 | 35.41 | C |
| ATOM | 977 | C | VAL | A | 155 | 9.094 | 66.905 | 5.336 | 1.00 | 33.10 | C |
| ATOM | 978 | O | VAL | A | 155 | 10.092 | 67.155 | 4.648 | 1.00 | 33.39 | O |
| ATOM | 979 | N | ARG | A | 156 | 8.130 | 66.086 | 4.931 | 1.00 | 33.25 | N |
| ATOM | 980 | CA | ARG | A | 156 | 8.190 | 65.429 | 3.606 | 1.00 | 34.45 | C |
| ATOM | 981 | CB | ARG | A | 156 | 6.992 | 64.490 | 3.396 | 1.00 | 33.21 | C |
| ATOM | 982 | CG | ARG | A | 156 | 6.999 | 63.221 | 4.219 | 1.00 | 33.42 | C |
| ATOM | 983 | CD | ARG | A | 156 | 5.778 | 62.320 | 3.937 | 1.00 | 34.78 | C |
| ATOM | 984 | NE | ARG | A | 156 | 5.644 | 62.064 | 2.494 | 1.00 | 35.47 | N |
| ATOM | 985 | CZ | ARG | A | 156 | 4.533 | 61.636 | 1.903 | 1.00 | 33.43 | C |
| ATOM | 986 | NH1 | ARG | A | 156 | 3.435 | 61.411 | 2.609 | 1.00 | 32.42 | N |
| ATOM | 987 | NH2 | ARG | A | 156 | 4.525 | 61.437 | 0.594 | 1.00 | 34.38 | N |
| ATOM | 988 | C | ARG | A | 156 | 8.211 | 66.491 | 2.501 | 1.00 | 34.92 | C |
| ATOM | 989 | O | ARG | A | 156 | 8.986 | 66.414 | 1.542 | 1.00 | 35.22 | O |
| ATOM | 990 | N | HIS | A | 157 | 7.369 | 67.501 | 2.650 | 1.00 | 36.13 | N |
| ATOM | 991 | CA | HIS | A | 157 | 7.351 | 68.588 | 1.686 | 1.00 | 36.56 | C |
| ATOM | 992 | CB | HIS | A | 157 | 6.299 | 69.629 | 2.048 | 1.00 | 37.38 | C |
| ATOM | 993 | CG | HIS | A | 157 | 6.362 | 70.863 | 1.197 | 1.00 | 39.12 | C |
| ATOM | 994 | ND1 | HIS | A | 157 | 7.005 | 72.014 | 1.608 | 1.00 | 41.07 | N |
| ATOM | 995 | CE1 | HIS | A | 157 | 6.921 | 72.926 | 0.658 | 1.00 | 39.75 | C |
| ATOM | 996 | NE2 | HIS | A | 157 | 6.249 | 72.407 | −0.358 | 1.00 | 40.88 | N |
| ATOM | 997 | CD2 | HIS | A | 157 | 5.873 | 71.124 | −0.041 | 1.00 | 38.81 | C |
| ATOM | 998 | C | HIS | A | 157 | 8.710 | 69.238 | 1.555 | 1.00 | 36.34 | C |
| ATOM | 999 | O | HIS | A | 157 | 9.178 | 69.475 | 0.435 | 1.00 | 36.86 | O |
| ATOM | 1000 | N | CYS | A | 158 | 9.354 | 69.542 | 2.683 | 1.00 | 36.43 | N |
| ATOM | 1001 | CA | CYS | A | 158 | 10.664 | 70.184 | 2.649 | 1.00 | 36.33 | C |
| ATOM | 1002 | CB | CYS | A | 158 | 11.177 | 70.492 | 4.065 | 1.00 | 36.27 | C |
| ATOM | 1003 | SG | CYS | A | 158 | 10.201 | 71.754 | 4.924 | 1.00 | 37.34 | S |
| ATOM | 1004 | C | CYS | A | 158 | 11.663 | 69.290 | 1.937 | 1.00 | 37.14 | C |
| ATOM | 1005 | O | CYS | A | 158 | 12.431 | 69.751 | 1.069 | 1.00 | 36.89 | O |
| ATOM | 1006 | N | HIS | A | 159 | 11.678 | 68.019 | 2.334 | 1.00 | 36.70 | N |
| ATOM | 1007 | CA | HIS | A | 159 | 12.624 | 67.055 | 1.784 | 1.00 | 38.40 | C |
| ATOM | 1008 | CB | HIS | A | 159 | 12.521 | 65.732 | 2.551 | 1.00 | 38.71 | C |
| ATOM | 1009 | CG | HIS | A | 159 | 13.136 | 65.801 | 3.916 | 1.00 | 44.25 | C |
| ATOM | 1010 | ND1 | HIS | A | 159 | 13.788 | 64.734 | 4.499 | 1.00 | 47.72 | N |
| ATOM | 1011 | CE1 | HIS | A | 159 | 14.258 | 65.103 | 5.681 | 1.00 | 49.12 | C |
| ATOM | 1012 | NE2 | HIS | A | 159 | 13.948 | 66.376 | 5.880 | 1.00 | 48.24 | N |
| ATOM | 1013 | CD2 | HIS | A | 159 | 13.238 | 66.834 | 4.798 | 1.00 | 47.23 | C |
| ATOM | 1014 | C | HIS | A | 159 | 12.392 | 66.881 | 0.277 | 1.00 | 38.37 | C |
| ATOM | 1015 | O | HIS | A | 159 | 13.337 | 66.781 | −0.481 | 1.00 | 36.82 | O |
| ATOM | 1016 | N | ASN | A | 160 | 11.128 | 66.926 | −0.127 | 1.00 | 39.53 | N |
| ATOM | 1017 | CA | ASN | A | 160 | 10.742 | 66.927 | −1.529 | 1.00 | 42.14 | C |
| ATOM | 1018 | CB | ASN | A | 160 | 9.239 | 67.045 | −1.629 | 1.00 | 43.88 | C |
| ATOM | 1019 | CG | ASN | A | 160 | 8.602 | 65.778 | −2.013 | 1.00 | 48.90 | C |
| ATOM | 1020 | OD1 | ASN | A | 160 | 8.727 | 64.765 | −1.312 | 1.00 | 53.59 | O |
| ATOM | 1021 | ND2 | ASN | A | 160 | 7.913 | 65.795 | −3.160 | 1.00 | 53.90 | N |
| ATOM | 1022 | C | ASN | A | 160 | 11.322 | 68.100 | −2.286 | 1.00 | 42.07 | C |
| ATOM | 1023 | O | ASN | A | 160 | 11.668 | 67.978 | −3.461 | 1.00 | 41.85 | O |
| ATOM | 1024 | N | CYS | A | 161 | 11.397 | 69.246 | −1.616 | 1.00 | 40.47 | N |
| ATOM | 1025 | CA | CYS | A | 161 | 11.884 | 70.467 | −2.225 | 1.00 | 39.41 | C |
| ATOM | 1026 | CB | CYS | A | 161 | 11.254 | 71.669 | −1.529 | 1.00 | 39.11 | C |
| ATOM | 1027 | SG | CYS | A | 161 | 9.498 | 71.835 | −1.845 | 1.00 | 40.42 | S |
| ATOM | 1028 | C | CYS | A | 161 | 13.391 | 70.551 | −2.129 | 1.00 | 38.90 | C |
| ATOM | 1029 | O | CYS | A | 161 | 13.979 | 71.555 | −2.518 | 1.00 | 39.21 | O |
| ATOM | 1030 | N | GLY | A | 162 | 14.022 | 69.510 | −1.596 | 1.00 | 38.48 | N |
| ATOM | 1031 | CA | GLY | A | 162 | 15.474 | 69.511 | −1.438 | 1.00 | 37.84 | C |
| ATOM | 1032 | C | GLY | A | 162 | 15.957 | 70.269 | −0.221 | 1.00 | 37.83 | C |
| ATOM | 1033 | O | GLY | A | 162 | 17.122 | 70.693 | −0.160 | 1.00 | 36.57 | O |
| ATOM | 1034 | N | VAL | A | 163 | 15.084 | 70.388 | 0.789 | 1.00 | 37.66 | N |
| ATOM | 1035 | CA | VAL | A | 163 | 15.420 | 71.145 | 2.007 | 1.00 | 37.08 | C |
| ATOM | 1036 | CB | VAL | A | 163 | 14.488 | 72.353 | 2.166 | 1.00 | 37.80 | C |
| ATOM | 1037 | CG1 | VAL | A | 163 | 14.748 | 73.082 | 3.511 | 1.00 | 38.56 | C |
| ATOM | 1038 | CG2 | VAL | A | 163 | 14.666 | 73.306 | 1.008 | 1.00 | 36.79 | C |
| ATOM | 1039 | C | VAL | A | 163 | 15.337 | 70.305 | 3.294 | 1.00 | 37.35 | C |
| ATOM | 1040 | O | VAL | A | 163 | 14.363 | 69.589 | 3.538 | 1.00 | 35.09 | O |
| ATOM | 1041 | N | LEU | A | 164 | 16.373 | 70.436 | 4.110 | 1.00 | 37.93 | N |
| ATOM | 1042 | CA | LEU | A | 164 | 16.468 | 69.790 | 5.405 | 1.00 | 38.78 | C |
| ATOM | 1043 | CB | LEU | A | 164 | 17.813 | 69.089 | 5.468 | 1.00 | 39.15 | C |
| ATOM | 1044 | CG | LEU | A | 164 | 18.083 | 68.153 | 6.625 | 1.00 | 41.81 | C |
| ATOM | 1045 | CD1 | LEU | A | 164 | 17.260 | 66.866 | 6.466 | 1.00 | 42.67 | C |
| ATOM | 1046 | CD2 | LEU | A | 164 | 19.553 | 67.843 | 6.711 | 1.00 | 43.27 | C |
| ATOM | 1047 | C | LEU | A | 164 | 16.382 | 70.911 | 6.472 | 1.00 | 38.81 | C |
| ATOM | 1048 | O | LEU | A | 164 | 17.209 | 71.834 | 6.474 | 1.00 | 38.57 | O |
| ATOM | 1049 | N | HIS | A | 165 | 15.387 | 70.830 | 7.357 | 1.00 | 38.45 | N |
| ATOM | 1050 | CA | HIS | A | 165 | 15.164 | 71.860 | 8.388 | 1.00 | 37.77 | C |
| ATOM | 1051 | CB | HIS | A | 165 | 13.758 | 71.731 | 8.991 | 1.00 | 37.59 | C |
| ATOM | 1052 | CG | HIS | A | 165 | 13.398 | 72.836 | 9.937 | 1.00 | 35.86 | C |
| ATOM | 1053 | ND1 | HIS | A | 165 | 13.867 | 72.891 | 11.229 | 1.00 | 33.70 | N |
| ATOM | 1054 | CE1 | HIS | A | 165 | 13.391 | 73.969 | 11.823 | 1.00 | 34.80 | C |
| ATOM | 1055 | NE2 | HIS | A | 165 | 12.628 | 74.617 | 10.959 | 1.00 | 37.29 | N |
| ATOM | 1056 | CD2 | HIS | A | 165 | 12.612 | 73.926 | 9.774 | 1.00 | 35.10 | C |
| ATOM | 1057 | C | HIS | A | 165 | 16.236 | 71.813 | 9.464 | 1.00 | 37.76 | C |
| ATOM | 1058 | O | HIS | A | 165 | 16.784 | 72.843 | 9.824 | 1.00 | 38.35 | O |
| ATOM | 1059 | N | ARG | A | 166 | 16.563 | 70.612 | 9.937 | 1.00 | 37.85 | N |
| ATOM | 1060 | CA | ARG | A | 166 | 17.615 | 70.390 | 10.933 | 1.00 | 38.53 | C |
| ATOM | 1061 | CB | ARG | A | 166 | 18.952 | 70.946 | 10.456 | 1.00 | 39.48 | C |
| ATOM | 1062 | CG | ARG | A | 166 | 19.500 | 70.338 | 9.178 | 1.00 | 42.23 | C |
| ATOM | 1063 | CD | ARG | A | 166 | 20.503 | 71.265 | 8.553 | 1.00 | 46.58 | C |
| ATOM | 1064 | NE | ARG | A | 166 | 21.839 | 70.788 | 8.808 | 1.00 | 50.91 | N |
| ATOM | 1065 | CZ | ARG | A | 166 | 22.933 | 71.523 | 8.743 | 1.00 | 50.31 | C |
| ATOM | 1066 | NH1 | ARG | A | 166 | 22.882 | 72.821 | 8.466 | 1.00 | 50.71 | N |
| ATOM | 1067 | NH2 | ARG | A | 166 | 24.091 | 70.941 | 8.972 | 1.00 | 50.70 | N |
| ATOM | 1068 | C | ARG | A | 166 | 17.370 | 70.951 | 12.331 | 1.00 | 38.67 | C |
| ATOM | 1069 | O | ARG | A | 166 | 18.243 | 70.839 | 13.184 | 1.00 | 39.30 | O |
| ATOM | 1070 | N | ASP | A | 167 | 16.222 | 71.567 | 12.569 | 1.00 | 38.24 | N |
| ATOM | 1071 | CA | ASP | A | 167 | 15.920 | 72.076 | 13.912 | 1.00 | 39.05 | C |
| ATOM | 1072 | CB | ASP | A | 167 | 16.297 | 73.567 | 13.972 | 1.00 | 40.02 | C |
| ATOM | 1073 | CG | ASP | A | 167 | 16.351 | 74.131 | 15.396 | 1.00 | 44.41 | C |
| ATOM | 1074 | OD1 | ASP | A | 167 | 16.656 | 73.391 | 16.374 | 1.00 | 44.09 | O |
| ATOM | 1075 | OD2 | ASP | A | 167 | 16.111 | 75.349 | 15.606 | 1.00 | 47.46 | O |
| ATOM | 1076 | C | ASP | A | 167 | 14.442 | 71.870 | 14.231 | 1.00 | 37.42 | C |
| ATOM | 1077 | O | ASP | A | 167 | 13.765 | 72.783 | 14.722 | 1.00 | 38.05 | O |
| ATOM | 1078 | N | ILE | A | 168 | 13.926 | 70.671 | 13.939 | 1.00 | 36.17 | N |
| ATOM | 1079 | CA | ILE | A | 168 | 12.516 | 70.380 | 14.201 | 1.00 | 34.82 | C |
| ATOM | 1080 | CB | ILE | A | 168 | 12.066 | 69.064 | 13.505 | 1.00 | 35.54 | C |
| ATOM | 1081 | CG1 | ILE | A | 168 | 12.125 | 69.196 | 11.976 | 1.00 | 34.25 | C |
| ATOM | 1082 | CD1 | ILE | A | 168 | 12.194 | 67.836 | 11.258 | 1.00 | 36.78 | C |
| ATOM | 1083 | CG2 | ILE | A | 168 | 10.663 | 68.692 | 13.951 | 1.00 | 34.38 | C |
| ATOM | 1084 | C | ILE | A | 168 | 12.306 | 70.252 | 15.708 | 1.00 | 34.43 | C |
| ATOM | 1085 | O | ILE | A | 168 | 12.914 | 69.409 | 16.350 | 1.00 | 32.59 | O |
| ATOM | 1086 | N | LYS | A | 169 | 11.436 | 71.091 | 16.260 | 1.00 | 33.96 | N |
| ATOM | 1087 | CA | LYS | A | 169 | 11.122 | 71.056 | 17.701 | 1.00 | 33.91 | C |
| ATOM | 1088 | CB | LYS | A | 169 | 12.281 | 71.647 | 18.511 | 1.00 | 34.23 | C |
| ATOM | 1089 | CG | LYS | A | 169 | 12.644 | 73.064 | 18.140 | 1.00 | 35.79 | C |
| ATOM | 1090 | CD | LYS | A | 169 | 13.822 | 73.538 | 18.954 | 1.00 | 40.57 | C |
| ATOM | 1091 | CE | LYS | A | 169 | 14.137 | 75.024 | 18.631 | 1.00 | 44.31 | C |
| ATOM | 1092 | NZ | LYS | A | 169 | 15.134 | 75.616 | 19.597 | 1.00 | 47.28 | N |
| ATOM | 1093 | C | LYS | A | 169 | 9.862 | 71.860 | 17.947 | 1.00 | 33.20 | C |
| ATOM | 1094 | O | LYS | A | 169 | 9.444 | 72.619 | 17.065 | 1.00 | 32.12 | O |
| ATOM | 1095 | N | ASP | A | 170 | 9.272 | 71.731 | 19.138 | 1.00 | 33.18 | N |
| ATOM | 1096 | CA | ASP | A | 170 | 8.021 | 72.433 | 19.438 | 1.00 | 35.25 | C |
| ATOM | 1097 | CB | ASP | A | 170 | 7.517 | 72.132 | 20.839 | 1.00 | 36.00 | C |
| ATOM | 1098 | CG | ASP | A | 170 | 8.582 | 72.296 | 21.895 | 1.00 | 38.87 | C |
| ATOM | 1099 | OD1 | ASP | A | 170 | 9.700 | 72.820 | 21.626 | 1.00 | 41.81 | O |
| ATOM | 1100 | OD2 | ASP | A | 170 | 8.358 | 71.892 | 23.042 | 1.00 | 42.14 | O |
| ATOM | 1101 | C | ASP | A | 170 | 8.075 | 73.934 | 19.226 | 1.00 | 35.41 | C |
| ATOM | 1102 | O | ASP | A | 170 | 7.118 | 74.510 | 18.717 | 1.00 | 35.26 | O |
| ATOM | 1103 | N | GLU | A | 171 | 9.204 | 74.550 | 19.570 | 1.00 | 36.73 | N |
| ATOM | 1104 | CA | GLU | A | 171 | 9.370 | 76.005 | 19.446 | 1.00 | 38.95 | C |
| ATOM | 1105 | CB | GLU | A | 171 | 10.703 | 76.462 | 20.053 | 1.00 | 40.09 | C |
| ATOM | 1106 | CG | GLU | A | 171 | 10.892 | 76.109 | 21.523 | 1.00 | 46.32 | C |
| ATOM | 1107 | CD | GLU | A | 171 | 12.296 | 76.436 | 22.017 | 1.00 | 53.18 | C |
| ATOM | 1108 | OE1 | GLU | A | 171 | 13.229 | 75.621 | 21.798 | 1.00 | 56.05 | O |
| ATOM | 1109 | OE2 | GLU | A | 171 | 12.474 | 77.511 | 22.636 | 1.00 | 57.82 | O |
| ATOM | 1110 | C | GLU | A | 171 | 9.340 | 76.438 | 17.983 | 1.00 | 38.68 | C |
| ATOM | 1111 | O | GLU | A | 171 | 9.000 | 77.583 | 17.678 | 1.00 | 38.39 | O |
| ATOM | 1112 | N | ASN | A | 172 | 9.716 | 75.531 | 17.080 | 1.00 | 37.88 | N |
| ATOM | 1113 | CA | ASN | A | 172 | 9.752 | 75.848 | 15.653 | 1.00 | 37.39 | C |
| ATOM | 1114 | CB | ASN | A | 172 | 11.022 | 75.288 | 15.019 | 1.00 | 37.23 | C |
| ATOM | 1115 | CG | ASN | A | 172 | 12.270 | 76.063 | 15.433 | 1.00 | 38.63 | C |
| ATOM | 1116 | OD1 | ASN | A | 172 | 12.195 | 77.241 | 15.769 | 1.00 | 38.90 | O |
| ATOM | 1117 | ND2 | ASN | A | 172 | 13.421 | 75.407 | 15.390 | 1.00 | 36.90 | N |
| ATOM | 1118 | C | ASN | A | 172 | 8.519 | 75.353 | 14.917 | 1.00 | 36.59 | C |
| ATOM | 1119 | O | ASN | A | 172 | 8.567 | 75.150 | 13.710 | 1.00 | 36.84 | O |
| ATOM | 1120 | N | ILE | A | 173 | 7.430 | 75.141 | 15.653 | 1.00 | 35.61 | N |
| ATOM | 1121 | CA | ILE | A | 173 | 6.143 | 74.742 | 15.085 | 1.00 | 35.39 | C |
| ATOM | 1122 | CB | ILE | A | 173 | 5.797 | 73.292 | 15.516 | 1.00 | 35.63 | C |
| ATOM | 1123 | CG1 | ILE | A | 173 | 6.798 | 72.283 | 14.897 | 1.00 | 36.07 | C |
| ATOM | 1124 | CD1 | ILE | A | 173 | 6.648 | 70.871 | 15.388 | 1.00 | 33.90 | C |
| ATOM | 1125 | CG2 | ILE | A | 173 | 4.356 | 72.954 | 15.161 | 1.00 | 35.62 | C |
| ATOM | 1126 | C | ILE | A | 173 | 5.023 | 75.691 | 15.548 | 1.00 | 36.51 | C |
| ATOM | 1127 | O | ILE | A | 173 | 4.796 | 75.863 | 16.767 | 1.00 | 35.35 | O |
| ATOM | 1128 | N | LEU | A | 174 | 4.319 | 76.286 | 14.588 | 1.00 | 36.38 | N |
| ATOM | 1129 | CA | LEU | A | 174 | 3.223 | 77.192 | 14.900 | 1.00 | 37.97 | C |
| ATOM | 1130 | CB | LEU | A | 174 | 3.307 | 78.506 | 14.107 | 1.00 | 38.37 | C |
| ATOM | 1131 | CG | LEU | A | 174 | 4.444 | 79.472 | 14.437 | 1.00 | 41.65 | C |
| ATOM | 1132 | CD1 | LEU | A | 174 | 4.357 | 80.715 | 13.531 | 1.00 | 44.42 | C |
| ATOM | 1133 | CD2 | LEU | A | 174 | 4.399 | 79.905 | 15.882 | 1.00 | 42.22 | C |
| ATOM | 1134 | C | LEU | A | 174 | 1.891 | 76.518 | 14.642 | 1.00 | 38.02 | C |
| ATOM | 1135 | O | LEU | A | 174 | 1.711 | 75.822 | 13.642 | 1.00 | 37.64 | O |
| ATOM | 1136 | N | ILE | A | 175 | 0.963 | 76.721 | 15.567 | 1.00 | 37.87 | N |
| ATOM | 1137 | CA | ILE | A | 175 | −0.379 | 76.199 | 15.417 | 1.00 | 38.43 | C |
| ATOM | 1138 | CB | ILE | A | 175 | −0.845 | 75.563 | 16.744 | 1.00 | 38.70 | C |
| ATOM | 1139 | CG1 | ILE | A | 175 | 0.148 | 74.510 | 17.228 | 1.00 | 38.66 | C |
| ATOM | 1140 | CD1 | ILE | A | 175 | −0.025 | 74.200 | 18.722 | 1.00 | 41.58 | C |
| ATOM | 1141 | CG2 | ILE | A | 175 | −2.241 | 74.971 | 16.609 | 1.00 | 36.19 | C |
| ATOM | 1142 | C | ILE | A | 175 | −1.342 | 77.313 | 14.997 | 1.00 | 40.30 | C |
| ATOM | 1143 | O | ILE | A | 175 | −1.522 | 78.307 | 15.716 | 1.00 | 41.15 | O |
| ATOM | 1144 | N | ASP | A | 176 | −1.969 | 77.144 | 13.840 | 1.00 | 41.47 | N |
| ATOM | 1145 | CA | ASP | A | 176 | −3.092 | 77.991 | 13.438 | 1.00 | 42.38 | C |
| ATOM | 1146 | CB | ASP | A | 176 | −3.337 | 77.853 | 11.926 | 1.00 | 42.29 | C |
| ATOM | 1147 | CG | ASP | A | 176 | −4.437 | 78.782 | 11.401 | 1.00 | 44.69 | C |
| ATOM | 1148 | OD1 | ASP | A | 176 | −5.440 | 79.033 | 12.113 | 1.00 | 46.71 | O |
| ATOM | 1149 | OD2 | ASP | A | 176 | −4.382 | 79.271 | 10.250 | 1.00 | 43.65 | O |
| ATOM | 1150 | C | ASP | A | 176 | −4.279 | 77.497 | 14.235 | 1.00 | 43.24 | C |
| ATOM | 1151 | O | ASP | A | 176 | −4.904 | 76.483 | 13.882 | 1.00 | 42.40 | O |
| ATOM | 1152 | N | LEU | A | 111 | −4.582 | 78.214 | 15.319 | 1.00 | 44.51 | N |
| ATOM | 1153 | CA | LEU | A | 111 | −5.612 | 77.803 | 16.281 | 1.00 | 45.60 | C |
| ATOM | 1154 | CB | LEU | A | 177 | −5.611 | 78.719 | 17.518 | 1.00 | 45.31 | C |
| ATOM | 1155 | CG | LEU | A | 177 | −4.338 | 78.689 | 18.362 | 1.00 | 45.46 | C |
| ATOM | 1156 | CD1 | LEU | A | 177 | −4.275 | 79.850 | 19.374 | 1.00 | 44.16 | C |
| ATOM | 1157 | CD2 | LEU | A | 177 | −4.247 | 77.335 | 19.066 | 1.00 | 44.99 | C |
| ATOM | 1158 | C | LEU | A | 177 | −7.019 | 77.691 | 15.708 | 1.00 | 46.83 | C |
| ATOM | 1159 | O | LEU | A | 177 | −7.793 | 76.840 | 16.145 | 1.00 | 47.74 | O |
| ATOM | 1160 | N | ASN | A | 178 | −7.348 | 78.535 | 14.737 | 1.00 | 47.91 | N |
| ATOM | 1161 | CA | ASN | A | 178 | −8.664 | 78.512 | 14.104 | 1.00 | 48.63 | C |
| ATOM | 1162 | CB | ASN | A | 178 | −8.886 | 79.810 | 13.316 | 1.00 | 49.80 | C |
| ATOM | 1163 | CG | ASN | A | 178 | −9.487 | 80.939 | 14.169 | 1.00 | 52.87 | C |
| ATOM | 1164 | OD1 | ASN | A | 178 | −9.966 | 80.712 | 15.287 | 1.00 | 55.06 | O |
| ATOM | 1165 | ND2 | ASN | A | 178 | −9.463 | 82.166 | 13.628 | 1.00 | 54.84 | N |
| ATOM | 1166 | C | ASN | A | 178 | −8.843 | 77.332 | 13.154 | 1.00 | 48.41 | C |
| ATOM | 1167 | O | ASN | A | 178 | −9.892 | 76.686 | 13.132 | 1.00 | 49.39 | O |
| ATOM | 1168 | N | ARG | A | 179 | −7.821 | 77.061 | 12.348 | 1.00 | 47.30 | N |
| ATOM | 1169 | CA | ARG | A | 179 | −7.907 | 75.998 | 11.353 | 1.00 | 45.90 | C |
| ATOM | 1170 | CB | ARG | A | 179 | −7.183 | 76.420 | 10.088 | 1.00 | 46.22 | C |
| ATOM | 1171 | CG | ARG | A | 179 | −7.790 | 77.611 | 9.403 | 1.00 | 47.89 | C |
| ATOM | 1172 | CD | ARG | A | 179 | −7.036 | 77.967 | 8.138 | 1.00 | 50.30 | C |
| ATOM | 1173 | NE | ARG | A | 179 | −7.672 | 79.037 | 7.378 | 1.00 | 54.18 | N |
| ATOM | 1174 | CZ | ARG | A | 179 | −8.825 | 78.919 | 6.715 | 1.00 | 56.15 | C |
| ATOM | 1175 | NH1 | ARG | A | 179 | −9.498 | 77.773 | 6.717 | 1.00 | 55.19 | N |
| ATOM | 1176 | NH2 | ARG | A | 179 | −9.309 | 79.958 | 6.042 | 1.00 | 57.24 | N |
| ATOM | 1177 | C | ARG | A | 179 | −7.356 | 74.653 | 11.839 | 1.00 | 44.78 | C |
| ATOM | 1178 | O | ARG | A | 179 | −7.604 | 73.614 | 11.208 | 1.00 | 44.31 | O |
| ATOM | 1179 | N | GLY | A | 180 | −6.612 | 74.667 | 12.946 | 1.00 | 42.54 | N |
| ATOM | 1180 | CA | GLY | A | 180 | −6.001 | 73.448 | 13.448 | 1.00 | 41.77 | C |
| ATOM | 1181 | C | GLY | A | 180 | −4.862 | 72.972 | 12.551 | 1.00 | 41.19 | C |
| ATOM | 1182 | O | GLY | A | 180 | −4.609 | 71.776 | 12.440 | 1.00 | 40.99 | O |
| ATOM | 1183 | N | GLU | A | 181 | −4.172 | 73.908 | 11.909 | 1.00 | 39.77 | N |
| ATOM | 1184 | CA | GLU | A | 181 | −3.105 | 73.549 | 10.986 | 1.00 | 39.51 | C |
| ATOM | 1185 | CB | GLU | A | 181 | −3.335 | 74.241 | 9.641 | 1.00 | 38.79 | C |
| ATOM | 1186 | CG | GLU | A | 181 | −4.438 | 73.608 | 8.809 | 1.00 | 39.75 | C |
| ATOM | 1187 | CD | GLU | A | 181 | −4.919 | 74.501 | 7.676 | 1.00 | 40.13 | C |
| ATOM | 1188 | OE1 | GLU | A | 181 | −4.195 | 75.443 | 7.326 | 1.00 | 42.55 | O |
| ATOM | 1189 | OE2 | GLU | A | 181 | −6.018 | 74.264 | 7.151 | 1.00 | 38.74 | O |
| ATOM | 1190 | C | GLU | A | 181 | −1.761 | 73.957 | 11.553 | 1.00 | 39.26 | C |
| ATOM | 1191 | O | GLU | A | 181 | −1.617 | 75.074 | 12.048 | 1.00 | 39.89 | O |
| ATOM | 1192 | N | LEU | A | 182 | −0.783 | 73.051 | 11.482 | 1.00 | 38.44 | N |
| ATOM | 1193 | CA | LEU | A | 182 | 0.567 | 73.323 | 11.966 | 1.00 | 37.99 | C |
| ATOM | 1194 | CB | LEU | A | 182 | 1.201 | 72.066 | 12.588 | 1.00 | 37.43 | C |
| ATOM | 1195 | CG | LEU | A | 182 | 0.947 | 71.895 | 14.094 | 1.00 | 38.02 | C |
| ATOM | 1196 | CD1 | LEU | A | 182 | −0.528 | 71.939 | 14.378 | 1.00 | 39.25 | C |
| ATOM | 1197 | CD2 | LEU | A | 182 | 1.546 | 70.567 | 14.578 | 1.00 | 35.43 | C |
| ATOM | 1198 | C | LEU | A | 182 | 1.448 | 73.854 | 10.857 | 1.00 | 37.83 | C |
| ATOM | 1199 | O | LEU | A | 182 | 1.256 | 73.519 | 9.688 | 1.00 | 37.14 | O |
| ATOM | 1200 | N | LYS | A | 183 | 2.417 | 74.681 | 11.235 | 1.00 | 37.37 | N |
| ATOM | 1201 | CA | LYS | A | 183 | 3.280 | 75.320 | 10.269 | 1.00 | 38.66 | C |
| ATOM | 1202 | CB | LYS | A | 183 | 2.756 | 76.721 | 9.919 | 1.00 | 39.51 | C |
| ATOM | 1203 | CG | LYS | A | 183 | 1.723 | 76.691 | 8.799 | 1.00 | 44.32 | C |
| ATOM | 1204 | CD | LYS | A | 183 | 1.157 | 78.081 | 8.560 | 1.00 | 50.71 | C |
| ATOM | 1205 | CE | LYS | A | 183 | 0.426 | 78.195 | 7.226 | 1.00 | 53.19 | C |
| ATOM | 1206 | NZ | LYS | A | 183 | −0.586 | 77.117 | 6.989 | 1.00 | 52.77 | N |
| ATOM | 1207 | C | LYS | A | 183 | 4.697 | 75.373 | 10.797 | 1.00 | 37.98 | C |
| ATOM | 1208 | O | LYS | A | 183 | 4.954 | 75.805 | 11.923 | 1.00 | 37.88 | O |
| ATOM | 1209 | N | LEU | A | 184 | 5.617 | 74.918 | 9.969 | 1.00 | 37.28 | N |
| ATOM | 1210 | CA | LEU | A | 184 | 7.015 | 74.830 | 10.333 | 1.00 | 37.45 | C |
| ATOM | 1211 | CB | LEU | A | 184 | 7.658 | 73.726 | 9.494 | 1.00 | 38.41 | C |
| ATOM | 1212 | CG | LEU | A | 184 | 9.013 | 73.126 | 9.811 | 1.00 | 42.44 | C |
| ATOM | 1213 | CD1 | LEU | A | 184 | 9.162 | 72.736 | 11.300 | 1.00 | 44.98 | C |
| ATOM | 1214 | CD2 | LEU | A | 184 | 9.156 | 71.900 | 8.895 | 1.00 | 44.78 | C |
| ATOM | 1215 | C | LEU | A | 184 | 7.689 | 76.195 | 10.135 | 1.00 | 37.17 | C |
| ATOM | 1216 | O | LEU | A | 184 | 7.411 | 76.886 | 9.159 | 1.00 | 34.75 | O |
| ATOM | 1217 | N | ILE | A | 185 | 8.546 | 76.590 | 11.085 | 1.00 | 37.08 | N |
| ATOM | 1218 | CA | ILE | A | 185 | 9.233 | 77.882 | 11.007 | 1.00 | 38.22 | C |
| ATOM | 1219 | CB | ILE | A | 185 | 8.589 | 78.967 | 11.964 | 1.00 | 38.24 | C |
| ATOM | 1220 | CG1 | ILE | A | 185 | 8.676 | 78.523 | 13.428 | 1.00 | 38.15 | C |
| ATOM | 1221 | CD1 | ILE | A | 185 | 8.508 | 79.649 | 14.460 | 1.00 | 40.09 | C |
| ATOM | 1222 | CG2 | ILE | A | 185 | 7.180 | 79.280 | 11.555 | 1.00 | 37.83 | C |
| ATOM | 1223 | C | ILE | A | 185 | 10.678 | 77.766 | 11.365 | 1.00 | 38.83 | C |
| ATOM | 1224 | O | ILE | A | 185 | 11.105 | 76.792 | 12.000 | 1.00 | 38.96 | O |
| ATOM | 1225 | N | ASP | A | 186 | 11.419 | 78.807 | 10.980 | 1.00 | 39.40 | N |
| ATOM | 1226 | CA | ASP | A | 186 | 12.822 | 78.985 | 11.315 | 1.00 | 40.27 | C |
| ATOM | 1227 | CB | ASP | A | 186 | 13.046 | 79.073 | 12.830 | 1.00 | 41.48 | C |
| ATOM | 1228 | CG | ASP | A | 186 | 14.441 | 79.582 | 13.178 | 1.00 | 45.31 | C |
| ATOM | 1229 | OD1 | ASP | A | 186 | 15.190 | 79.992 | 12.255 | 1.00 | 47.25 | O |
| ATOM | 1230 | OD2 | ASP | A | 186 | 14.885 | 79.588 | 14.351 | 1.00 | 50.71 | O |
| ATOM | 1231 | C | ASP | A | 186 | 13.803 | 78.013 | 10.648 | 1.00 | 40.90 | C |
| ATOM | 1232 | O | ASP | A | 186 | 14.343 | 77.096 | 11.285 | 1.00 | 40.21 | O |
| ATOM | 1233 | N | PHE | A | 187 | 14.087 | 78.292 | 9.378 | 1.00 | 40.98 | N |
| ATOM | 1234 | CA | PHE | A | 187 | 15.042 | 77.522 | 8.591 | 1.00 | 42.42 | C |
| ATOM | 1235 | CB | PHE | A | 187 | 14.602 | 77.517 | 7.140 | 1.00 | 41.27 | C |
| ATOM | 1236 | CG | PHE | A | 187 | 13.394 | 76.662 | 6.891 | 1.00 | 41.11 | C |
| ATOM | 1237 | CD1 | PHE | A | 187 | 12.129 | 77.128 | 7.202 | 1.00 | 40.65 | C |
| ATOM | 1238 | CE1 | PHE | A | 187 | 11.000 | 76.342 | 6.977 | 1.00 | 39.93 | C |
| ATOM | 1239 | CZ | PHE | A | 187 | 11.131 | 75.078 | 6.444 | 1.00 | 40.45 | C |
| ATOM | 1240 | CE2 | PHE | A | 187 | 12.398 | 74.586 | 6.128 | 1.00 | 38.34 | C |
| ATOM | 1241 | CD2 | PHE | A | 187 | 13.522 | 75.373 | 6.349 | 1.00 | 40.39 | C |
| ATOM | 1242 | C | PHE | A | 187 | 16.476 | 78.031 | 8.711 | 1.00 | 43.76 | C |
| ATOM | 1243 | O | PHE | A | 187 | 17.346 | 77.647 | 7.927 | 1.00 | 44.80 | O |
| ATOM | 1244 | N | GLY | A | 188 | 16.723 | 78.868 | 9.716 | 1.00 | 44.55 | N |
| ATOM | 1245 | CA | GLY | A | 188 | 18.034 | 79.449 | 9.940 | 1.00 | 45.36 | C |
| ATOM | 1246 | C | GLY | A | 188 | 19.156 | 78.493 | 10.252 | 1.00 | 45.97 | C |
| ATOM | 1247 | O | GLY | A | 188 | 20.320 | 78.870 | 10.168 | 1.00 | 46.90 | O |
| ATOM | 1248 | N | SER | A | 189 | 18.830 | 77.260 | 10.631 | 1.00 | 46.13 | N |
| ATOM | 1249 | CA | SER | A | 189 | 19.853 | 76.240 | 10.866 | 1.00 | 45.91 | C |
| ATOM | 1250 | CB | SER | A | 189 | 19.725 | 75.652 | 12.280 | 1.00 | 46.57 | C |
| ATOM | 1251 | OG | SER | A | 189 | 19.539 | 76.674 | 13.258 | 1.00 | 51.43 | O |
| ATOM | 1252 | C | SER | A | 189 | 19.742 | 75.111 | 9.825 | 1.00 | 44.92 | C |
| ATOM | 1253 | O | SER | A | 189 | 20.356 | 74.051 | 9.977 | 1.00 | 43.79 | O |
| ATOM | 1254 | N | GLY | A | 190 | 18.948 | 75.337 | 8.784 | 1.00 | 44.05 | N |
| ATOM | 1255 | CA | GLY | A | 190 | 18.720 | 74.310 | 7.784 | 1.00 | 43.67 | C |
| ATOM | 1256 | C | GLY | A | 190 | 19.851 | 74.120 | 6.783 | 1.00 | 43.35 | C |
| ATOM | 1257 | O | GLY | A | 190 | 20.908 | 74.764 | 6.862 | 1.00 | 41.72 | O |
| ATOM | 1258 | N | ALA | A | 191 | 19.614 | 73.222 | 5.825 | 1.00 | 42.84 | N |
| ATOM | 1259 | CA | ALA | A | 191 | 20.584 | 72.942 | 4.769 | 1.00 | 41.99 | C |
| ATOM | 1260 | CB | ALA | A | 191 | 21.722 | 72.067 | 5.294 | 1.00 | 41.84 | C |
| ATOM | 1261 | C | ALA | A | 191 | 19.927 | 72.305 | 3.551 | 1.00 | 42.18 | C |
| ATOM | 1262 | O | ALA | A | 191 | 18.779 | 71.813 | 3.608 | 1.00 | 41.24 | O |
| ATOM | 1263 | N | LEU | A | 192 | 20.637 | 72.347 | 2.428 | 1.00 | 42.32 | N |
| ATOM | 1264 | CA | LEU | A | 192 | 20.170 | 71.649 | 1.236 | 1.00 | 42.24 | C |
| ATOM | 1265 | CB | LEU | A | 192 | 21.059 | 71.977 | 0.031 | 1.00 | 43.21 | C |
| ATOM | 1266 | CG | LEU | A | 192 | 21.088 | 73.455 | −0.389 | 1.00 | 46.28 | C |
| ATOM | 1267 | CD1 | LEU | A | 192 | 22.271 | 73.763 | −1.328 | 1.00 | 49.62 | C |
| ATOM | 1268 | CD2 | LEU | A | 192 | 19.778 | 73.889 | −1.025 | 1.00 | 46.71 | C |
| ATOM | 1269 | C | LEU | A | 192 | 20.244 | 70.179 | 1.589 | 1.00 | 41.12 | C |
| ATOM | 1270 | O | LEU | A | 192 | 21.187 | 69.742 | 2.270 | 1.00 | 39.72 | O |
| ATOM | 1271 | N | LEU | A | 193 | 19.227 | 69.428 | 1.190 | 1.00 | 41.80 | N |
| ATOM | 1272 | CA | LEU | A | 193 | 19.237 | 67.983 | 1.401 | 1.00 | 43.29 | C |
| ATOM | 1273 | CB | LEU | A | 193 | 17.870 | 67.405 | 1.066 | 1.00 | 43.47 | C |
| ATOM | 1274 | CG | LEU | A | 193 | 17.658 | 65.896 | 1.213 | 1.00 | 45.93 | C |
| ATOM | 1275 | CD1 | LEU | A | 193 | 17.805 | 65.456 | 2.671 | 1.00 | 45.54 | C |
| ATOM | 1276 | CD2 | LEU | A | 193 | 16.279 | 65.518 | 0.652 | 1.00 | 46.41 | C |
| ATOM | 1277 | C | LEU | A | 193 | 20.306 | 67.331 | 0.512 | 1.00 | 43.97 | C |
| ATOM | 1278 | O | LEU | A | 193 | 20.386 | 67.649 | −0.667 | 1.00 | 44.14 | O |
| ATOM | 1279 | N | LYS | A | 194 | 21.110 | 66.434 | 1.084 | 1.00 | 44.53 | N |
| ATOM | 1280 | CA | LYS | A | 194 | 22.106 | 65.663 | 0.340 | 1.00 | 45.14 | C |
| ATOM | 1281 | CB | LYS | A | 194 | 23.500 | 66.291 | 0.450 | 1.00 | 45.29 | C |
| ATOM | 1282 | CG | LYS | A | 194 | 24.054 | 66.305 | 1.860 | 1.00 | 44.80 | C |
| ATOM | 1283 | CD | LYS | A | 194 | 25.300 | 67.144 | 1.961 | 1.00 | 45.08 | C |
| ATOM | 1284 | CE | LYS | A | 194 | 25.991 | 66.854 | 3.284 | 1.00 | 46.87 | C |
| ATOM | 1285 | NZ | LYS | A | 194 | 27.243 | 67.643 | 3.464 | 1.00 | 48.08 | N |
| ATOM | 1286 | C | LYS | A | 194 | 22.134 | 64.272 | 0.920 | 1.00 | 45.51 | C |
| ATOM | 1287 | O | LYS | A | 194 | 21.615 | 64.054 | 2.026 | 1.00 | 45.00 | O |
| ATOM | 1288 | N | ASP | A | 195 | 22.745 | 63.335 | 0.188 | 1.00 | 45.31 | N |
| ATOM | 1289 | CA | ASP | A | 195 | 22.779 | 61.933 | 0.609 | 1.00 | 45.82 | C |
| ATOM | 1290 | CB | ASP | A | 195 | 22.653 | 60.999 | −0.601 | 1.00 | 46.48 | C |
| ATOM | 1291 | CG | ASP | A | 195 | 21.330 | 61.124 | −1.303 | 1.00 | 47.68 | C |
| ATOM | 1292 | OD1 | ASP | A | 195 | 20.279 | 60.858 | −0.678 | 1.00 | 50.02 | O |
| ATOM | 1293 | OD2 | ASP | A | 195 | 21.243 | 61.469 | −2.499 | 1.00 | 49.94 | O |
| ATOM | 1294 | C | ASP | A | 195 | 24.038 | 61.607 | 1.384 | 1.00 | 45.41 | C |
| ATOM | 1295 | O | ASP | A | 195 | 24.161 | 60.519 | 1.950 | 1.00 | 46.43 | O |
| ATOM | 1296 | N | THR | A | 196 | 24.984 | 62.538 | 1.385 | 1.00 | 45.63 | N |
| ATOM | 1297 | CA | THR | A | 196 | 26.259 | 62.371 | 2.083 | 1.00 | 46.00 | C |
| ATOM | 1298 | CB | THR | A | 196 | 27.394 | 63.091 | 1.322 | 1.00 | 45.69 | C |
| ATOM | 1299 | OG1 | THR | A | 196 | 26.951 | 64.387 | 0.899 | 1.00 | 44.12 | O |
| ATOM | 1300 | CG2 | THR | A | 196 | 27.728 | 62.348 | 0.026 | 1.00 | 46.47 | C |
| ATOM | 1301 | C | THR | A | 196 | 26.211 | 62.902 | 3.518 | 1.00 | 46.75 | C |
| ATOM | 1302 | O | THR | A | 196 | 25.283 | 63.616 | 3.886 | 1.00 | 46.70 | O |
| ATOM | 1303 | N | VAL | A | 197 | 27.237 | 62.569 | 4.302 | 1.00 | 47.32 | N |
| ATOM | 1304 | CA | VAL | A | 197 | 27.294 | 62.912 | 5.713 | 1.00 | 48.22 | C |
| ATOM | 1305 | CB | VAL | A | 197 | 28.440 | 62.174 | 6.437 | 1.00 | 48.78 | C |
| ATOM | 1306 | CG1 | VAL | A | 197 | 29.801 | 62.699 | 6.003 | 1.00 | 50.58 | C |
| ATOM | 1307 | CG2 | VAL | A | 197 | 28.282 | 62.289 | 7.956 | 1.00 | 49.66 | C |
| ATOM | 1308 | C | VAL | A | 197 | 27.366 | 64.409 | 5.965 | 1.00 | 48.10 | C |
| ATOM | 1309 | O | VAL | A | 197 | 27.949 | 65.152 | 5.182 | 1.00 | 47.85 | O |
| ATOM | 1310 | N | TYR | A | 198 | 26.717 | 64.842 | 7.046 | 1.00 | 47.69 | N |
| ATOM | 1311 | CA | TYR | A | 198 | 26.810 | 66.212 | 7.531 | 1.00 | 47.39 | C |
| ATOM | 1312 | CB | TYR | A | 198 | 25.437 | 66.722 | 7.984 | 1.00 | 46.27 | C |
| ATOM | 1313 | CG | TYR | A | 198 | 24.412 | 66.951 | 6.891 | 1.00 | 43.05 | C |
| ATOM | 1314 | CD1 | TYR | A | 198 | 23.574 | 65.924 | 6.464 | 1.00 | 40.17 | C |
| ATOM | 1315 | CE1 | TYR | A | 198 | 22.631 | 66.123 | 5.466 | 1.00 | 39.03 | C |
| ATOM | 1316 | CZ | TYR | A | 198 | 22.490 | 67.368 | 4.904 | 1.00 | 38.13 | C |
| ATOM | 1317 | OH | TYR | A | 198 | 21.539 | 67.577 | 3.933 | 1.00 | 36.97 | O |
| ATOM | 1318 | CE2 | TYR | A | 198 | 23.293 | 68.421 | 5.317 | 1.00 | 39.95 | C |
| ATOM | 1319 | CD2 | TYR | A | 198 | 24.256 | 68.204 | 6.312 | 1.00 | 41.73 | C |
| ATOM | 1320 | C | TYR | A | 198 | 27.753 | 66.211 | 8.729 | 1.00 | 48.60 | C |
| ATOM | 1321 | O | TYR | A | 198 | 27.657 | 65.349 | 9.597 | 1.00 | 48.27 | O |
| ATOM | 1322 | N | THR | A | 199 | 28.658 | 67.183 | 8.775 | 1.00 | 50.37 | N |
| ATOM | 1323 | CA | THR | A | 199 | 29.619 | 67.304 | 9.875 | 1.00 | 52.48 | C |
| ATOM | 1324 | CB | THR | A | 199 | 31.079 | 67.267 | 9.364 | 1.00 | 52.38 | C |
| ATOM | 1325 | OG1 | THR | A | 199 | 31.242 | 68.240 | 8.318 | 1.00 | 53.03 | O |
| ATOM | 1326 | CG2 | THR | A | 199 | 31.393 | 65.936 | 8.714 | 1.00 | 53.00 | C |
| ATOM | 1327 | C | THR | A | 199 | 29.409 | 68.606 | 10.636 | 1.00 | 53.92 | C |
| ATOM | 1328 | O | THR | A | 199 | 30.172 | 68.924 | 11.545 | 1.00 | 53.86 | O |
| ATOM | 1329 | N | ASP | A | 200 | 28.381 | 69.359 | 10.253 | 1.00 | 56.06 | N |
| ATOM | 1330 | CA | ASP | A | 200 | 28.005 | 70.568 | 10.977 | 1.00 | 58.11 | C |
| ATOM | 1331 | CB | ASP | A | 200 | 28.067 | 71.798 | 10.062 | 1.00 | 58.64 | C |
| ATOM | 1332 | CG | ASP | A | 200 | 26.971 | 71.802 | 9.017 | 1.00 | 59.95 | C |
| ATOM | 1333 | OD1 | ASP | A | 200 | 26.266 | 72.826 | 8.884 | 1.00 | 61.08 | O |
| ATOM | 1334 | OD2 | ASP | A | 200 | 26.739 | 70.813 | 8.279 | 1.00 | 63.15 | O |
| ATOM | 1335 | C | ASP | A | 200 | 26.602 | 70.424 | 11.539 | 1.00 | 59.05 | C |
| ATOM | 1336 | O | ASP | A | 200 | 25.751 | 69.737 | 10.957 | 1.00 | 58.97 | O |
| ATOM | 1337 | N | PHE | A | 201 | 26.365 | 71.091 | 12.664 | 1.00 | 60.22 | N |
| ATOM | 1338 | CA | PHE | A | 201 | 25.061 | 71.089 | 13.315 | 1.00 | 61.47 | C |
| ATOM | 1339 | CB | PHE | A | 201 | 24.847 | 69.790 | 14.094 | 1.00 | 61.39 | C |
| ATOM | 1340 | CG | PHE | A | 201 | 23.526 | 69.717 | 14.805 | 1.00 | 61.76 | C |
| ATOM | 1341 | CD1 | PHE | A | 201 | 22.342 | 69.550 | 14.085 | 1.00 | 62.43 | C |
| ATOM | 1342 | CE1 | PHE | A | 201 | 21.110 | 69.475 | 14.741 | 1.00 | 62.41 | C |
| ATOM | 1343 | CZ | PHE | A | 201 | 21.064 | 69.560 | 16.131 | 1.00 | 62.12 | C |
| ATOM | 1344 | CE2 | PHE | A | 201 | 22.242 | 69.727 | 16.856 | 1.00 | 61.55 | C |
| ATOM | 1345 | CD2 | PHE | A | 201 | 23.464 | 69.804 | 16.190 | 1.00 | 61.34 | C |
| ATOM | 1346 | C | PHE | A | 201 | 24.957 | 72.286 | 14.245 | 1.00 | 62.42 | C |
| ATOM | 1347 | O | PHE | A | 201 | 25.712 | 72.411 | 15.214 | 1.00 | 62.75 | O |
| ATOM | 1348 | N | ASP | A | 202 | 24.012 | 73.158 | 13.934 | 1.00 | 63.58 | N |
| ATOM | 1349 | CA | ASP | A | 202 | 23.820 | 74.406 | 14.651 | 1.00 | 64.74 | C |
| ATOM | 1350 | CB | ASP | A | 202 | 24.100 | 75.583 | 13.704 | 1.00 | 65.58 | C |
| ATOM | 1351 | CG | ASP | A | 202 | 23.966 | 76.930 | 14.388 | 1.00 | 69.34 | C |
| ATOM | 1352 | OD1 | ASP | A | 202 | 24.626 | 77.141 | 15.440 | 1.00 | 71.91 | O |
| ATOM | 1353 | OD2 | ASP | A | 202 | 23.207 | 77.831 | 13.950 | 1.00 | 72.83 | O |
| ATOM | 1354 | C | ASP | A | 202 | 22.397 | 74.467 | 15.198 | 1.00 | 64.11 | C |
| ATOM | 1355 | O | ASP | A | 202 | 21.920 | 75.524 | 15.600 | 1.00 | 64.30 | O |
| ATOM | 1356 | N | GLY | A | 203 | 21.716 | 73.324 | 15.202 | 1.00 | 63.47 | N |
| ATOM | 1357 | CA | GLY | A | 203 | 20.358 | 73.250 | 15.712 | 1.00 | 62.03 | C |
| ATOM | 1358 | C | GLY | A | 203 | 20.346 | 72.947 | 17.200 | 1.00 | 60.94 | C |
| ATOM | 1359 | O | GLY | A | 203 | 21.392 | 72.972 | 17.854 | 1.00 | 61.08 | O |
| ATOM | 1360 | N | THR | A | 204 | 19.158 | 72.643 | 17.727 | 1.00 | 59.86 | N |
| ATOM | 1361 | CA | THR | A | 204 | 18.975 | 72.364 | 19.158 | 1.00 | 58.03 | C |
| ATOM | 1362 | CB | THR | A | 204 | 17.481 | 72.402 | 19.547 | 1.00 | 57.90 | C |
| ATOM | 1363 | OG1 | THR | A | 204 | 16.900 | 73.630 | 19.090 | 1.00 | 56.77 | O |
| ATOM | 1364 | CG2 | THR | A | 204 | 17.332 | 72.488 | 21.079 | 1.00 | 57.65 | C |
| ATOM | 1365 | C | THR | A | 204 | 19.574 | 71.032 | 19.575 | 1.00 | 57.57 | C |
| ATOM | 1366 | O | THR | A | 204 | 19.196 | 69.966 | 19.047 | 1.00 | 56.94 | O |
| ATOM | 1367 | N | ARG | A | 205 | 20.487 | 71.106 | 20.545 | 1.00 | 56.60 | N |
| ATOM | 1368 | CA | ARG | A | 205 | 21.238 | 69.959 | 21.022 | 1.00 | 56.09 | C |
| ATOM | 1369 | CB | ARG | A | 205 | 22.204 | 70.417 | 22.124 | 1.00 | 56.67 | C |
| ATOM | 1370 | CG | ARG | A | 205 | 22.870 | 69.291 | 22.879 | 1.00 | 59.97 | C |
| ATOM | 1371 | CD | ARG | A | 205 | 24.127 | 69.719 | 23.631 | 1.00 | 63.64 | C |
| ATOM | 1372 | NE | ARG | A | 205 | 25.317 | 69.608 | 22.785 | 1.00 | 64.42 | N |
| ATOM | 1373 | CZ | ARG | A | 205 | 26.049 | 68.501 | 22.667 | 1.00 | 65.48 | C |
| ATOM | 1374 | NH1 | ARG | A | 205 | 25.712 | 67.410 | 23.340 | 1.00 | 65.75 | N |
| ATOM | 1375 | NH2 | ARG | A | 205 | 27.114 | 68.476 | 21.872 | 1.00 | 64.31 | N |
| ATOM | 1376 | C | ARG | A | 205 | 20.360 | 68.784 | 21.503 | 1.00 | 55.41 | C |
| ATOM | 1377 | O | ARG | A | 205 | 20.536 | 67.630 | 21.069 | 1.00 | 55.27 | O |
| ATOM | 1378 | N | VAL | A | 206 | 19.420 | 69.077 | 22.400 | 1.00 | 54.06 | N |
| ATOM | 1379 | CA | VAL | A | 206 | 18.634 | 68.037 | 23.067 | 1.00 | 52.40 | C |
| ATOM | 1380 | CB | VAL | A | 206 | 17.704 | 68.640 | 24.178 | 1.00 | 52.71 | C |
| ATOM | 1381 | CG1 | VAL | A | 206 | 18.516 | 69.018 | 25.416 | 1.00 | 50.99 | C |
| ATOM | 1382 | CG2 | VAL | A | 206 | 16.919 | 69.844 | 23.636 | 1.00 | 51.73 | C |
| ATOM | 1383 | C | VAL | A | 206 | 17.799 | 67.291 | 22.048 | 1.00 | 51.99 | C |
| ATOM | 1384 | O | VAL | A | 206 | 17.219 | 66.257 | 22.363 | 1.00 | 52.27 | O |
| ATOM | 1385 | N | TYR | A | 207 | 17.731 | 67.834 | 20.830 | 1.00 | 50.50 | N |
| ATOM | 1386 | CA | TYR | A | 207 | 17.001 | 67.202 | 19.738 | 1.00 | 49.94 | C |
| ATOM | 1387 | CB | TYR | A | 207 | 16.126 | 68.236 | 19.021 | 1.00 | 49.34 | C |
| ATOM | 1388 | CG | TYR | A | 207 | 14.759 | 68.542 | 19.600 | 1.00 | 48.61 | C |
| ATOM | 1389 | CD1 | TYR | A | 207 | 14.604 | 69.438 | 20.679 | 1.00 | 49.28 | C |
| ATOM | 1390 | CE1 | TYR | A | 207 | 13.314 | 69.753 | 21.194 | 1.00 | 48.65 | C |
| ATOM | 1391 | CZ | TYR | A | 207 | 12.182 | 69.164 | 20.590 | 1.00 | 50.59 | C |
| ATOM | 1392 | OH | TYR | A | 207 | 10.901 | 69.447 | 21.042 | 1.00 | 47.38 | O |
| ATOM | 1393 | CE2 | TYR | A | 207 | 12.332 | 68.284 | 19.488 | 1.00 | 48.14 | C |
| ATOM | 1394 | CD2 | TYR | A | 207 | 13.605 | 67.999 | 19.007 | 1.00 | 48.95 | C |
| ATOM | 1395 | C | TYR | A | 207 | 17.982 | 66.571 | 18.718 | 1.00 | 49.22 | C |
| ATOM | 1396 | O | TYR | A | 207 | 17.560 | 66.165 | 17.621 | 1.00 | 48.88 | O |
| ATOM | 1397 | N | SER | A | 208 | 19.269 | 66.529 | 19.085 | 1.00 | 48.13 | N |
| ATOM | 1398 | CA | SER | A | 208 | 20.361 | 66.030 | 18.231 | 1.00 | 47.87 | C |
| ATOM | 1399 | CB | SER | A | 208 | 21.667 | 66.791 | 18.496 | 1.00 | 48.10 | C |
| ATOM | 1400 | OG | SER | A | 208 | 22.280 | 66.316 | 19.688 | 1.00 | 49.78 | O |
| ATOM | 1401 | C | SER | A | 208 | 20.620 | 64.566 | 18.503 | 1.00 | 46.42 | C |
| ATOM | 1402 | O | SER | A | 208 | 20.531 | 64.110 | 19.656 | 1.00 | 46.94 | O |
| ATOM | 1403 | N | PRO | A | 209 | 20.941 | 63.826 | 17.449 | 1.00 | 44.93 | N |
| ATOM | 1404 | CA | PRO | A | 209 | 21.043 | 62.374 | 17.545 | 1.00 | 43.05 | C |
| ATOM | 1405 | CB | PRO | A | 209 | 20.979 | 61.948 | 16.083 | 1.00 | 43.11 | C |
| ATOM | 1406 | CG | PRO | A | 209 | 21.596 | 63.062 | 15.366 | 1.00 | 43.72 | C |
| ATOM | 1407 | CD | PRO | A | 209 | 21.165 | 64.293 | 16.070 | 1.00 | 45.04 | C |
| ATOM | 1408 | C | PRO | A | 209 | 22.334 | 61.918 | 18.200 | 1.00 | 42.08 | C |
| ATOM | 1409 | O | PRO | A | 209 | 23.303 | 62.675 | 18.235 | 1.00 | 40.92 | O |
| ATOM | 1410 | N | PRO | A | 210 | 22.355 | 60.685 | 18.705 | 1.00 | 41.24 | N |
| ATOM | 1411 | CA | PRO | A | 210 | 23.546 | 60.167 | 19.374 | 1.00 | 42.13 | C |
| ATOM | 1412 | CB | PRO | A | 210 | 23.117 | 58.762 | 19.830 | 1.00 | 41.13 | C |
| ATOM | 1413 | CG | PRO | A | 210 | 21.980 | 58.403 | 18.942 | 1.00 | 41.77 | C |
| ATOM | 1414 | CD | PRO | A | 210 | 21.270 | 59.693 | 18.669 | 1.00 | 40.59 | C |
| ATOM | 1415 | C | PRO | A | 210 | 24.768 | 60.119 | 18.442 | 1.00 | 43.19 | C |
| ATOM | 1416 | O | PRO | A | 210 | 25.884 | 60.302 | 18.942 | 1.00 | 42.91 | O |
| ATOM | 1417 | N | GLU | A | 211 | 24.567 | 59.901 | 17.138 | 1.00 | 43.80 | N |
| ATOM | 1418 | CA | GLU | A | 211 | 25.683 | 59.896 | 16.184 | 1.00 | 45.23 | C |
| ATOM | 1419 | CB | GLU | A | 211 | 25.253 | 59.400 | 14.780 | 1.00 | 44.59 | C |
| ATOM | 1420 | CG | GLU | A | 211 | 24.227 | 60.279 | 14.079 | 1.00 | 42.32 | C |
| ATOM | 1421 | CD | GLU | A | 211 | 22.796 | 59.821 | 14.334 | 1.00 | 41.06 | C |
| ATOM | 1422 | OE1 | GLU | A | 211 | 22.529 | 59.217 | 15.394 | 1.00 | 38.90 | O |
| ATOM | 1423 | OE2 | GLU | A | 211 | 21.940 | 60.065 | 13.460 | 1.00 | 38.76 | O |
| ATOM | 1424 | C | GLU | A | 211 | 26.354 | 61.263 | 16.095 | 1.00 | 46.56 | C |
| ATOM | 1425 | O | GLU | A | 211 | 27.563 | 61.353 | 15.883 | 1.00 | 46.98 | O |
| ATOM | 1426 | N | TRP | A | 212 | 25.585 | 62.331 | 16.284 | 1.00 | 48.34 | N |
| ATOM | 1427 | CA | TRP | A | 212 | 26.184 | 63.658 | 16.339 | 1.00 | 50.36 | C |
| ATOM | 1428 | CB | TRP | A | 212 | 25.147 | 64.769 | 16.186 | 1.00 | 50.50 | C |
| ATOM | 1429 | CG | TRP | A | 212 | 25.742 | 66.114 | 16.495 | 1.00 | 52.39 | C |
| ATOM | 1430 | CD1 | TRP | A | 212 | 25.599 | 66.830 | 17.652 | 1.00 | 53.01 | C |
| ATOM | 1431 | NE1 | TRP | A | 212 | 26.318 | 67.999 | 17.579 | 1.00 | 53.68 | N |
| ATOM | 1432 | CE2 | TRP | A | 212 | 26.962 | 68.052 | 16.368 | 1.00 | 53.34 | C |
| ATOM | 1433 | CD2 | TRP | A | 212 | 26.626 | 66.877 | 15.661 | 1.00 | 52.72 | C |
| ATOM | 1434 | CE3 | TRP | A | 212 | 27.159 | 66.692 | 14.373 | 1.00 | 52.54 | C |
| ATOM | 1435 | CZ3 | TRP | A | 212 | 27.992 | 67.675 | 13.842 | 1.00 | 52.69 | C |
| ATOM | 1436 | CH2 | TRP | A | 212 | 28.306 | 68.832 | 14.575 | 1.00 | 52.71 | C |
| ATOM | 1437 | CZ2 | TRP | A | 212 | 27.802 | 69.040 | 15.833 | 1.00 | 53.56 | C |
| ATOM | 1438 | C | TRP | A | 212 | 26.996 | 63.835 | 17.622 | 1.00 | 51.79 | C |
| ATOM | 1439 | O | TRP | A | 212 | 28.118 | 64.342 | 17.588 | 1.00 | 52.13 | O |
| ATOM | 1440 | N | ILE | A | 213 | 26.435 | 63.388 | 18.743 | 1.00 | 53.62 | N |
| ATOM | 1441 | CA | ILE | A | 213 | 27.095 | 63.496 | 20.048 | 1.00 | 55.72 | C |
| ATOM | 1442 | CB | ILE | A | 213 | 26.195 | 62.917 | 21.183 | 1.00 | 55.41 | C |
| ATOM | 1443 | CG1 | ILE | A | 213 | 24.804 | 63.568 | 21.202 | 1.00 | 56.07 | C |
| ATOM | 1444 | CD1 | ILE | A | 213 | 24.816 | 65.083 | 21.258 | 1.00 | 57.43 | C |
| ATOM | 1445 | CG2 | ILE | A | 213 | 26.874 | 63.055 | 22.525 | 1.00 | 56.33 | C |
| ATOM | 1446 | C | ILE | A | 213 | 28.440 | 62.771 | 20.050 | 1.00 | 57.10 | C |
| ATOM | 1447 | O | ILE | A | 213 | 29.461 | 63.335 | 20.447 | 1.00 | 57.22 | O |
| ATOM | 1448 | N | ARG | A | 214 | 28.416 | 61.524 | 19.591 | 1.00 | 58.27 | N |
| ATOM | 1449 | CA | ARG | A | 214 | 29.559 | 60.635 | 19.650 | 1.00 | 59.99 | C |
| ATOM | 1450 | CB | ARG | A | 214 | 29.083 | 59.190 | 19.585 | 1.00 | 60.48 | C |
| ATOM | 1451 | CG | ARG | A | 214 | 28.391 | 58.721 | 20.837 | 1.00 | 64.19 | C |
| ATOM | 1452 | CD | ARG | A | 214 | 28.138 | 57.237 | 20.844 | 1.00 | 68.93 | C |
| ATOM | 1453 | NE | ARG | A | 214 | 29.398 | 56.501 | 20.865 | 1.00 | 73.42 | N |
| ATOM | 1454 | CZ | ARG | A | 214 | 29.499 | 55.185 | 21.015 | 1.00 | 76.10 | C |
| ATOM | 1455 | NH1 | ARG | A | 214 | 28.405 | 54.439 | 21.161 | 1.00 | 76.91 | N |
| ATOM | 1456 | NH2 | ARG | A | 214 | 30.697 | 54.609 | 21.013 | 1.00 | 76.26 | N |
| ATOM | 1457 | C | ARG | A | 214 | 30.579 | 60.864 | 18.546 | 1.00 | 59.96 | C |
| ATOM | 1458 | O | ARG | A | 214 | 31.774 | 60.803 | 18.812 | 1.00 | 60.37 | O |
| ATOM | 1459 | N | TYR | A | 215 | 30.116 | 61.106 | 17.318 | 1.00 | 59.65 | N |
| ATOM | 1460 | CA | TYR | A | 215 | 31.018 | 61.161 | 16.159 | 1.00 | 59.46 | C |
| ATOM | 1461 | CB | TYR | A | 215 | 30.751 | 59.997 | 15.196 | 1.00 | 59.78 | C |
| ATOM | 1462 | CG | TYR | A | 215 | 30.624 | 58.648 | 15.858 | 1.00 | 61.91 | C |
| ATOM | 1463 | CD1 | TYR | A | 215 | 31.657 | 58.120 | 16.639 | 1.00 | 63.80 | C |
| ATOM | 1464 | CE1 | TYR | A | 215 | 31.532 | 56.877 | 17.247 | 1.00 | 64.01 | C |
| ATOM | 1465 | CZ | TYR | A | 215 | 30.370 | 56.151 | 17.070 | 1.00 | 65.13 | C |
| ATOM | 1466 | OH | TYR | A | 215 | 30.228 | 54.910 | 17.657 | 1.00 | 66.40 | O |
| ATOM | 1467 | CE2 | TYR | A | 215 | 29.346 | 56.647 | 16.288 | 1.00 | 64.30 | C |
| ATOM | 1468 | CD2 | TYR | A | 215 | 29.475 | 57.885 | 15.692 | 1.00 | 63.31 | C |
| ATOM | 1469 | C | TYR | A | 215 | 30.984 | 62.453 | 15.364 | 1.00 | 58.84 | C |
| ATOM | 1470 | O | TYR | A | 215 | 31.672 | 62.556 | 14.356 | 1.00 | 58.96 | O |
| ATOM | 1471 | N | HIS | A | 216 | 30.189 | 63.431 | 15.791 | 1.00 | 57.99 | N |
| ATOM | 1472 | CA | HIS | A | 216 | 30.018 | 64.666 | 15.020 | 1.00 | 57.44 | C |
| ATOM | 1473 | CB | HIS | A | 216 | 31.238 | 65.583 | 15.198 | 1.00 | 58.54 | C |
| ATOM | 1474 | CG | HIS | A | 216 | 31.302 | 66.231 | 16.547 | 1.00 | 62.71 | C |
| ATOM | 1475 | ND1 | HIS | A | 216 | 30.780 | 67.486 | 16.793 | 1.00 | 65.61 | N |
| ATOM | 1476 | CE1 | HIS | A | 216 | 30.965 | 67.796 | 18.065 | 1.00 | 67.49 | C |
| ATOM | 1477 | NE2 | HIS | A | 216 | 31.591 | 66.788 | 18.655 | 1.00 | 67.79 | N |
| ATOM | 1478 | CD2 | HIS | A | 216 | 31.808 | 65.793 | 17.730 | 1.00 | 65.96 | C |
| ATOM | 1479 | C | HIS | A | 216 | 29.721 | 64.415 | 13.524 | 1.00 | 55.81 | C |
| ATOM | 1480 | O | HIS | A | 216 | 30.212 | 65.135 | 12.653 | 1.00 | 55.97 | O |
| ATOM | 1481 | N | ARG | A | 217 | 28.910 | 63.395 | 13.243 | 1.00 | 53.48 | N |
| ATOM | 1482 | CA | ARG | A | 217 | 28.502 | 63.041 | 11.881 | 1.00 | 51.48 | C |
| ATOM | 1483 | CB | ARG | A | 217 | 29.335 | 61.864 | 11.347 | 1.00 | 51.91 | C |
| ATOM | 1484 | CG | ARG | A | 217 | 30.818 | 62.132 | 11.121 | 1.00 | 54.97 | C |
| ATOM | 1485 | CD | ARG | A | 217 | 31.688 | 60.860 | 11.180 | 1.00 | 59.48 | C |
| ATOM | 1486 | NE | ARG | A | 217 | 31.581 | 60.059 | 9.957 | 1.00 | 63.43 | N |
| ATOM | 1487 | CZ | ARG | A | 217 | 32.061 | 60.411 | 8.751 | 1.00 | 64.73 | C |
| ATOM | 1488 | NH1 | ARG | A | 217 | 32.700 | 61.569 | 8.577 | 1.00 | 66.07 | N |
| ATOM | 1489 | NH2 | ARG | A | 217 | 31.892 | 59.602 | 7.709 | 1.00 | 63.46 | N |
| ATOM | 1490 | C | ARG | A | 217 | 27.054 | 62.581 | 11.923 | 1.00 | 48.94 | C |
| ATOM | 1491 | O | ARG | A | 217 | 26.641 | 61.939 | 12.884 | 1.00 | 48.53 | O |
| ATOM | 1492 | N | TYR | A | 218 | 26.300 | 62.893 | 10.875 | 1.00 | 45.99 | N |
| ATOM | 1493 | CA | TYR | A | 218 | 24.938 | 62.394 | 10.722 | 1.00 | 43.71 | C |
| ATOM | 1494 | CB | TYR | A | 218 | 23.976 | 63.126 | 11.694 | 1.00 | 42.32 | C |
| ATOM | 1495 | CG | TYR | A | 218 | 23.830 | 64.587 | 11.395 | 1.00 | 40.14 | C |
| ATOM | 1496 | CD1 | TYR | A | 218 | 24.708 | 65.529 | 11.937 | 1.00 | 39.79 | C |
| ATOM | 1497 | CE1 | TYR | A | 218 | 24.574 | 66.882 | 11.628 | 1.00 | 42.32 | C |
| ATOM | 1498 | CZ | TYR | A | 218 | 23.562 | 67.298 | 10.770 | 1.00 | 41.14 | C |
| ATOM | 1499 | OH | TYR | A | 218 | 23.412 | 68.630 | 10.464 | 1.00 | 43.32 | O |
| ATOM | 1500 | CE2 | TYR | A | 218 | 22.680 | 66.375 | 10.224 | 1.00 | 39.68 | C |
| ATOM | 1501 | CD2 | TYR | A | 218 | 22.828 | 65.031 | 10.539 | 1.00 | 39.80 | C |
| ATOM | 1502 | C | TYR | A | 218 | 24.448 | 62.520 | 9.279 | 1.00 | 42.58 | C |
| ATOM | 1503 | O | TYR | A | 218 | 24.959 | 63.323 | 8.492 | 1.00 | 42.66 | O |
| ATOM | 1504 | N | HIS | A | 219 | 23.434 | 61.732 | 8.947 | 1.00 | 41.95 | N |
| ATOM | 1505 | CA | HIS | A | 219 | 22.769 | 61.849 | 7.658 | 1.00 | 40.52 | C |
| ATOM | 1506 | CB | HIS | A | 219 | 22.655 | 60.465 | 7.030 | 1.00 | 41.07 | C |
| ATOM | 1507 | CG | HIS | A | 219 | 23.984 | 59.906 | 6.614 | 1.00 | 42.12 | C |
| ATOM | 1508 | ND1 | HIS | A | 219 | 24.497 | 60.080 | 5.344 | 1.00 | 43.99 | N |
| ATOM | 1509 | CE1 | HIS | A | 219 | 25.692 | 59.523 | 5.273 | 1.00 | 42.45 | C |
| ATOM | 1510 | NE2 | HIS | A | 219 | 25.982 | 59.010 | 6.455 | 1.00 | 43.76 | N |
| ATOM | 1511 | CD2 | HIS | A | 219 | 24.935 | 59.246 | 7.317 | 1.00 | 41.90 | C |
| ATOM | 1512 | C | HIS | A | 219 | 21.404 | 62.521 | 7.843 | 1.00 | 39.33 | C |
| ATOM | 1513 | O | HIS | A | 219 | 20.779 | 62.370 | 8.889 | 1.00 | 38.64 | O |
| ATOM | 1514 | N | GLY | A | 220 | 20.965 | 63.270 | 6.836 | 1.00 | 38.11 | N |
| ATOM | 1515 | CA | GLY | A | 220 | 19.784 | 64.103 | 6.950 | 1.00 | 37.81 | C |
| ATOM | 1516 | C | GLY | A | 220 | 18.527 | 63.394 | 7.405 | 1.00 | 38.27 | C |
| ATOM | 1517 | O | GLY | A | 220 | 17.931 | 63.745 | 8.429 | 1.00 | 37.53 | O |
| ATOM | 1518 | N | ARG | A | 221 | 18.122 | 62.386 | 6.647 | 1.00 | 38.11 | N |
| ATOM | 1519 | CA | ARG | A | 221 | 16.855 | 61.717 | 6.895 | 1.00 | 38.50 | C |
| ATOM | 1520 | CB | ARG | A | 221 | 16.542 | 60.729 | 5.767 | 1.00 | 40.47 | C |
| ATOM | 1521 | CG | ARG | A | 221 | 16.585 | 61.401 | 4.388 | 1.00 | 45.37 | C |
| ATOM | 1522 | CD | ARG | A | 221 | 16.575 | 60.448 | 3.185 | 1.00 | 51.09 | C |
| ATOM | 1523 | NE | ARG | A | 221 | 16.584 | 61.200 | 1.919 | 1.00 | 53.82 | N |
| ATOM | 1524 | CZ | ARG | A | 221 | 17.690 | 61.495 | 1.222 | 1.00 | 55.73 | C |
| ATOM | 1525 | NH1 | ARG | A | 221 | 18.894 | 61.099 | 1.646 | 1.00 | 56.00 | N |
| ATOM | 1526 | NH2 | ARG | A | 221 | 17.594 | 62.164 | 0.075 | 1.00 | 55.14 | N |
| ATOM | 1527 | C | ARG | A | 221 | 16.824 | 61.050 | 8.256 | 1.00 | 37.41 | C |
| ATOM | 1528 | O | ARG | A | 221 | 15.873 | 61.254 | 9.013 | 1.00 | 37.16 | O |
| ATOM | 1529 | N | SER | A | 222 | 17.858 | 60.290 | 8.597 | 1.00 | 36.13 | N |
| ATOM | 1530 | CA | SER | A | 222 | 17.836 | 59.563 | 9.863 | 1.00 | 35.73 | C |
| ATOM | 1531 | CB | SER | A | 222 | 18.900 | 58.448 | 9.890 | 1.00 | 34.73 | C |
| ATOM | 1532 | OG | SER | A | 222 | 20.215 | 58.968 | 9.772 | 1.00 | 36.77 | O |
| ATOM | 1533 | C | SER | A | 222 | 17.941 | 60.519 | 11.069 | 1.00 | 35.26 | C |
| ATOM | 1534 | O | SER | A | 222 | 17.365 | 60.250 | 12.137 | 1.00 | 35.32 | O |
| ATOM | 1535 | N | ALA | A | 223 | 18.647 | 61.633 | 10.899 | 1.00 | 34.89 | N |
| ATOM | 1536 | CA | ALA | A | 223 | 18.743 | 62.643 | 11.958 | 1.00 | 34.34 | C |
| ATOM | 1537 | CB | ALA | A | 223 | 19.847 | 63.660 | 11.666 | 1.00 | 32.42 | C |
| ATOM | 1538 | C | ALA | A | 223 | 17.399 | 63.350 | 12.101 | 1.00 | 34.25 | C |
| ATOM | 1539 | O | ALA | A | 223 | 16.992 | 63.726 | 13.214 | 1.00 | 33.94 | O |
| ATOM | 1540 | N | ALA | A | 224 | 16.699 | 63.523 | 10.983 | 1.00 | 33.64 | N |
| ATOM | 1541 | CA | ALA | A | 224 | 15.384 | 64.152 | 11.033 | 1.00 | 32.93 | C |
| ATOM | 1542 | CB | ALA | A | 224 | 14.862 | 64.456 | 9.651 | 1.00 | 33.29 | C |
| ATOM | 1543 | C | ALA | A | 224 | 14.410 | 63.269 | 11.812 | 1.00 | 33.44 | C |
| ATOM | 1544 | O | ALA | A | 224 | 13.645 | 63.770 | 12.651 | 1.00 | 33.58 | O |
| ATOM | 1545 | N | VAL | A | 225 | 14.455 | 61.962 | 11.562 | 1.00 | 32.40 | N |
| ATOM | 1546 | CA | VAL | A | 225 | 13.569 | 61.003 | 12.228 | 1.00 | 31.69 | C |
| ATOM | 1547 | CB | VAL | A | 225 | 13.724 | 59.574 | 11.622 | 1.00 | 32.53 | C |
| ATOM | 1548 | CG1 | VAL | A | 225 | 13.083 | 58.504 | 12.507 | 1.00 | 30.93 | C |
| ATOM | 1549 | CG2 | VAL | A | 225 | 13.123 | 59.544 | 10.219 | 1.00 | 32.75 | C |
| ATOM | 1550 | C | VAL | A | 225 | 13.856 | 60.988 | 13.740 | 1.00 | 32.12 | C |
| ATOM | 1551 | O | VAL | A | 225 | 12.943 | 60.876 | 14.552 | 1.00 | 31.44 | O |
| ATOM | 1552 | N | TRP | A | 226 | 15.125 | 61.117 | 14.110 | 1.00 | 31.95 | N |
| ATOM | 1553 | CA | TRP | A | 226 | 15.476 | 61.173 | 15.530 | 1.00 | 32.47 | C |
| ATOM | 1554 | CB | TRP | A | 226 | 16.990 | 61.279 | 15.721 | 1.00 | 33.06 | C |
| ATOM | 1555 | CG | TRP | A | 226 | 17.322 | 61.494 | 17.183 | 1.00 | 32.56 | C |
| ATOM | 1556 | CD1 | TRP | A | 226 | 17.334 | 62.682 | 17.851 | 1.00 | 32.49 | C |
| ATOM | 1557 | NE1 | TRP | A | 226 | 17.660 | 62.479 | 19.173 | 1.00 | 32.64 | N |
| ATOM | 1558 | CE2 | TRP | A | 226 | 17.834 | 61.134 | 19.383 | 1.00 | 31.73 | C |
| ATOM | 1559 | CD2 | TRP | A | 226 | 17.631 | 60.485 | 18.148 | 1.00 | 31.24 | C |
| ATOM | 1560 | CE3 | TRP | A | 226 | 17.757 | 59.089 | 18.094 | 1.00 | 32.89 | C |
| ATOM | 1561 | CZ3 | TRP | A | 226 | 18.096 | 58.391 | 19.261 | 1.00 | 32.32 | C |
| ATOM | 1562 | CH2 | TRP | A | 226 | 18.286 | 59.080 | 20.478 | 1.00 | 33.34 | C |
| ATOM | 1563 | CZ2 | TRP | A | 226 | 18.157 | 60.444 | 20.552 | 1.00 | 32.43 | C |
| ATOM | 1564 | C | TRP | A | 226 | 14.754 | 62.372 | 16.178 | 1.00 | 32.00 | C |
| ATOM | 1565 | O | TRP | A | 226 | 14.071 | 62.224 | 17.192 | 1.00 | 31.94 | O |
| ATOM | 1566 | N | SER | A | 227 | 14.872 | 63.546 | 15.558 | 1.00 | 31.65 | N |
| ATOM | 1567 | CA | SER | A | 227 | 14.217 | 64.752 | 16.073 | 1.00 | 31.57 | C |
| ATOM | 1568 | CB | SER | A | 227 | 14.611 | 65.982 | 15.259 | 1.00 | 31.61 | C |
| ATOM | 1569 | OG | SER | A | 227 | 13.916 | 66.048 | 14.016 | 1.00 | 33.45 | O |
| ATOM | 1570 | C | SER | A | 227 | 12.695 | 64.599 | 16.161 | 1.00 | 31.31 | C |
| ATOM | 1571 | O | SER | A | 227 | 12.052 | 65.151 | 17.072 | 1.00 | 30.56 | O |
| ATOM | 1572 | N | LEU | A | 228 | 12.124 | 63.841 | 15.229 | 1.00 | 30.36 | N |
| ATOM | 1573 | CA | LEU | A | 228 | 10.701 | 63.545 | 15.217 | 1.00 | 30.48 | C |
| ATOM | 1574 | CB | LEU | A | 228 | 10.300 | 62.865 | 13.901 | 1.00 | 30.69 | C |
| ATOM | 1575 | CG | LEU | A | 228 | 10.325 | 63.767 | 12.661 | 1.00 | 31.45 | C |
| ATOM | 1576 | CD1 | LEU | A | 228 | 10.069 | 62.947 | 11.389 | 1.00 | 30.81 | C |
| ATOM | 1577 | CD2 | LEU | A | 228 | 9.321 | 64.917 | 12.784 | 1.00 | 30.15 | C |
| ATOM | 1578 | C | LEU | A | 228 | 10.315 | 62.661 | 16.394 | 1.00 | 29.84 | C |
| ATOM | 1579 | O | LEU | A | 228 | 9.227 | 62.815 | 16.958 | 1.00 | 30.50 | O |
| ATOM | 1580 | N | GLY | A | 229 | 11.206 | 61.751 | 16.765 | 1.00 | 29.67 | N |
| ATOM | 1581 | CA | GLY | A | 229 | 11.008 | 60.895 | 17.920 | 1.00 | 29.67 | C |
| ATOM | 1582 | C | GLY | A | 229 | 10.994 | 61.723 | 19.208 | 1.00 | 30.52 | C |
| ATOM | 1583 | O | GLY | A | 229 | 10.169 | 61.486 | 20.105 | 1.00 | 28.98 | O |
| ATOM | 1584 | N | ILE | A | 230 | 11.920 | 62.670 | 19.307 | 1.00 | 30.54 | N |
| ATOM | 1585 | CA | ILE | A | 230 | 11.986 | 63.587 | 20.459 | 1.00 | 31.11 | C |
| ATOM | 1586 | CB | ILE | A | 230 | 13.199 | 64.564 | 20.334 | 1.00 | 31.68 | C |
| ATOM | 1587 | CG1 | ILE | A | 230 | 14.526 | 63.792 | 20.281 | 1.00 | 30.92 | C |
| ATOM | 1588 | CD1 | ILE | A | 230 | 14.824 | 62.992 | 21.546 | 1.00 | 30.66 | C |
| ATOM | 1589 | CG2 | ILE | A | 230 | 13.229 | 65.553 | 21.533 | 1.00 | 30.61 | C |
| ATOM | 1590 | C | ILE | A | 230 | 10.693 | 64.397 | 20.532 | 1.00 | 31.56 | C |
| ATOM | 1591 | O | ILE | A | 230 | 10.050 | 64.488 | 21.596 | 1.00 | 31.09 | O |
| ATOM | 1592 | N | LEU | A | 231 | 10.289 | 64.928 | 19.373 | 1.00 | 30.97 | N |
| ATOM | 1593 | CA | LEU | A | 231 | 9.050 | 65.711 | 19.257 | 1.00 | 30.14 | C |
| ATOM | 1594 | CB | LEU | A | 231 | 8.894 | 66.239 | 17.828 | 1.00 | 30.10 | C |
| ATOM | 1595 | CG | LEU | A | 231 | 7.627 | 67.043 | 17.556 | 1.00 | 32.25 | C |
| ATOM | 1596 | CD1 | LEU | A | 231 | 7.733 | 68.372 | 18.310 | 1.00 | 30.47 | C |
| ATOM | 1597 | CD2 | LEU | A | 231 | 7.419 | 67.246 | 16.065 | 1.00 | 30.92 | C |
| ATOM | 1598 | C | LEU | A | 231 | 7.798 | 64.950 | 19.689 | 1.00 | 30.59 | C |
| ATOM | 1599 | O | LEU | A | 231 | 6.949 | 65.484 | 20.439 | 1.00 | 30.81 | O |
| ATOM | 1600 | N | LEU | A | 232 | 7.655 | 63.721 | 19.210 | 1.00 | 29.58 | N |
| ATOM | 1601 | CA | LEU | A | 232 | 6.499 | 62.916 | 19.552 | 1.00 | 30.41 | C |
| ATOM | 1602 | CB | LEU | A | 232 | 6.470 | 61.609 | 18.745 | 1.00 | 30.17 | C |
| ATOM | 1603 | CG | LEU | A | 232 | 5.301 | 60.642 | 19.033 | 1.00 | 30.99 | C |
| ATOM | 1604 | CD1 | LEU | A | 232 | 3.947 | 61.346 | 18.919 | 1.00 | 33.55 | C |
| ATOM | 1605 | CD2 | LEU | A | 232 | 5.359 | 59.465 | 18.073 | 1.00 | 31.95 | C |
| ATOM | 1606 | C | LEU | A | 232 | 6.439 | 62.630 | 21.062 | 1.00 | 30.78 | C |
| ATOM | 1607 | O | LEU | A | 232 | 5.371 | 62.721 | 21.667 | 1.00 | 31.42 | O |
| ATOM | 1608 | N | TYR | A | 233 | 7.571 | 62.272 | 21.650 | 1.00 | 30.02 | N |
| ATOM | 1609 | CA | TYR | A | 233 | 7.646 | 62.042 | 23.103 | 1.00 | 30.55 | C |
| ATOM | 1610 | CB | TYR | A | 233 | 9.068 | 61.662 | 23.526 | 1.00 | 30.26 | C |
| ATOM | 1611 | CG | TYR | A | 233 | 9.209 | 61.373 | 25.008 | 1.00 | 28.93 | C |
| ATOM | 1612 | CD1 | TYR | A | 233 | 9.255 | 62.416 | 25.930 | 1.00 | 29.15 | C |
| ATOM | 1613 | CE1 | TYR | A | 233 | 9.353 | 62.171 | 27.311 | 1.00 | 28.65 | C |
| ATOM | 1614 | CZ | TYR | A | 233 | 9.407 | 60.882 | 27.769 | 1.00 | 31.81 | C |
| ATOM | 1615 | OH | TYR | A | 233 | 9.505 | 60.681 | 29.132 | 1.00 | 36.02 | O |
| ATOM | 1616 | CE2 | TYR | A | 233 | 9.365 | 59.801 | 26.878 | 1.00 | 30.59 | C |
| ATOM | 1617 | CD2 | TYR | A | 233 | 9.267 | 60.058 | 25.486 | 1.00 | 28.47 | C |
| ATOM | 1618 | C | TYR | A | 233 | 7.216 | 63.306 | 23.834 | 1.00 | 31.37 | C |
| ATOM | 1619 | O | TYR | A | 233 | 6.416 | 63.250 | 24.769 | 1.00 | 32.79 | O |
| ATOM | 1620 | N | ASP | A | 234 | 7.762 | 64.434 | 23.407 | 1.00 | 31.52 | N |
| ATOM | 1621 | CA | ASP | A | 234 | 7.411 | 65.750 | 23.934 | 1.00 | 33.52 | C |
| ATOM | 1622 | CB | ASP | A | 234 | 8.156 | 66.833 | 23.162 | 1.00 | 34.26 | C |
| ATOM | 1623 | CG | ASP | A | 234 | 7.951 | 68.224 | 23.745 | 1.00 | 37.82 | C |
| ATOM | 1624 | OD1 | ASP | A | 234 | 8.206 | 68.450 | 24.956 | 1.00 | 39.31 | O |
| ATOM | 1625 | OD2 | ASP | A | 234 | 7.531 | 69.156 | 23.030 | 1.00 | 39.97 | O |
| ATOM | 1626 | C | ASP | A | 234 | 5.923 | 66.023 | 23.923 | 1.00 | 34.29 | C |
| ATOM | 1627 | O | ASP | A | 234 | 5.368 | 66.524 | 24.931 | 1.00 | 35.28 | O |
| ATOM | 1628 | N | MET | A | 235 | 5.258 | 65.695 | 22.810 | 1.00 | 33.03 | N |
| ATOM | 1629 | CA | MET | A | 235 | 3.819 | 65.904 | 22.713 | 1.00 | 33.96 | C |
| ATOM | 1630 | CB | MET | A | 235 | 3.293 | 65.625 | 21.305 | 1.00 | 33.12 | C |
| ATOM | 1631 | CG | MET | A | 235 | 3.641 | 66.708 | 20.286 | 1.00 | 36.42 | C |
| ATOM | 1632 | SD | MET | A | 235 | 2.965 | 66.246 | 18.692 | 1.00 | 39.55 | S |
| ATOM | 1633 | CE | MET | A | 235 | 4.174 | 65.260 | 18.147 | 1.00 | 43.23 | C |
| ATOM | 1634 | C | MET | A | 235 | 3.020 | 65.078 | 23.703 | 1.00 | 33.96 | C |
| ATOM | 1635 | O | MET | A | 235 | 2.133 | 65.607 | 24.337 | 1.00 | 35.00 | O |
| ATOM | 1636 | N | VAL | A | 236 | 3.322 | 63.787 | 23.816 | 1.00 | 34.07 | N |
| ATOM | 1637 | CA | VAL | A | 236 | 2.518 | 62.893 | 24.660 | 1.00 | 35.02 | C |
| ATOM | 1638 | CB | VAL | A | 236 | 2.405 | 61.476 | 24.055 | 1.00 | 35.33 | C |
| ATOM | 1639 | CG1 | VAL | A | 236 | 1.757 | 61.562 | 22.673 | 1.00 | 34.30 | C |
| ATOM | 1640 | CG2 | VAL | A | 236 | 3.763 | 60.805 | 23.937 | 1.00 | 33.11 | C |
| ATOM | 1641 | C | VAL | A | 236 | 2.955 | 62.843 | 26.129 | 1.00 | 35.63 | C |
| ATOM | 1642 | O | VAL | A | 236 | 2.225 | 62.326 | 26.970 | 1.00 | 35.58 | O |
| ATOM | 1643 | N | CYS | A | 237 | 4.131 | 63.389 | 26.432 | 1.00 | 36.11 | N |
| ATOM | 1644 | CA | CYS | A | 237 | 4.642 | 63.383 | 27.814 | 1.00 | 36.98 | C |
| ATOM | 1645 | CB | CYS | A | 237 | 5.972 | 62.630 | 27.909 | 1.00 | 36.35 | C |
| ATOM | 1646 | SG | CYS | A | 237 | 5.796 | 60.844 | 27.757 | 1.00 | 38.34 | S |
| ATOM | 1647 | C | CYS | A | 237 | 4.790 | 64.778 | 28.416 | 1.00 | 37.40 | C |
| ATOM | 1648 | O | CYS | A | 237 | 4.983 | 64.907 | 29.628 | 1.00 | 38.17 | O |
| ATOM | 1649 | N | GLY | A | 238 | 4.722 | 65.812 | 27.576 | 1.00 | 36.49 | N |
| ATOM | 1650 | CA | GLY | A | 238 | 4.791 | 67.183 | 28.045 | 1.00 | 36.34 | C |
| ATOM | 1651 | C | GLY | A | 238 | 6.186 | 67.717 | 28.259 | 1.00 | 37.64 | C |
| ATOM | 1652 | O | GLY | A | 238 | 6.353 | 68.842 | 28.719 | 1.00 | 37.75 | O |
| ATOM | 1653 | N | ASP | A | 239 | 7.198 | 66.916 | 27.939 | 1.00 | 38.11 | N |
| ATOM | 1654 | CA | ASP | A | 239 | 8.580 | 67.369 | 28.009 | 1.00 | 39.21 | C |
| ATOM | 1655 | CB | ASP | A | 239 | 9.056 | 67.339 | 29.458 | 1.00 | 40.74 | C |
| ATOM | 1656 | CG | ASP | A | 239 | 10.214 | 68.302 | 29.735 | 1.00 | 45.40 | C |
| ATOM | 1657 | OD1 | ASP | A | 239 | 10.586 | 69.135 | 28.867 | 1.00 | 49.01 | O |
| ATOM | 1658 | OD2 | ASP | A | 239 | 10.822 | 68.274 | 30.828 | 1.00 | 50.33 | O |
| ATOM | 1659 | C | ASP | A | 239 | 9.418 | 66.434 | 27.142 | 1.00 | 39.28 | C |
| ATOM | 1660 | O | ASP | A | 239 | 8.957 | 65.354 | 26.769 | 1.00 | 39.18 | O |
| ATOM | 1661 | N | ILE | A | 240 | 10.630 | 66.856 | 26.809 | 1.00 | 39.54 | N |
| ATOM | 1662 | CA | ILE | A | 240 | 11.529 | 66.066 | 25.983 | 1.00 | 39.95 | C |
| ATOM | 1663 | CB | ILE | A | 240 | 12.641 | 66.964 | 25.440 | 1.00 | 40.83 | C |
| ATOM | 1664 | CG1 | ILE | A | 240 | 13.306 | 67.740 | 26.578 | 1.00 | 41.83 | C |
| ATOM | 1665 | CD1 | ILE | A | 240 | 14.455 | 68.627 | 26.125 | 1.00 | 44.95 | C |
| ATOM | 1666 | CG2 | ILE | A | 240 | 12.092 | 67.911 | 24.344 | 1.00 | 39.77 | C |
| ATOM | 1667 | C | ILE | A | 240 | 12.106 | 64.916 | 26.827 | 1.00 | 40.26 | C |
| ATOM | 1668 | O | ILE | A | 240 | 12.187 | 65.046 | 28.049 | 1.00 | 40.89 | O |
| ATOM | 1669 | N | PRO | A | 241 | 12.470 | 63.791 | 26.210 | 1.00 | 40.14 | N |
| ATOM | 1670 | CA | PRO | A | 241 | 12.941 | 62.620 | 26.971 | 1.00 | 41.03 | C |
| ATOM | 1671 | CB | PRO | A | 241 | 12.879 | 61.494 | 25.932 | 1.00 | 40.79 | C |
| ATOM | 1672 | CG | PRO | A | 241 | 13.150 | 62.187 | 24.622 | 1.00 | 39.44 | C |
| ATOM | 1673 | CD | PRO | A | 241 | 12.444 | 63.518 | 24.757 | 1.00 | 39.54 | C |
| ATOM | 1674 | C | PRO | A | 241 | 14.361 | 62.737 | 27.548 | 1.00 | 42.89 | C |
| ATOM | 1675 | O | PRO | A | 241 | 14.639 | 62.109 | 28.571 | 1.00 | 42.98 | O |
| ATOM | 1676 | N | PHE | A | 242 | 15.243 | 63.508 | 26.912 | 1.00 | 44.80 | N |
| ATOM | 1677 | CA | PHE | A | 242 | 16.644 | 63.555 | 27.340 | 1.00 | 46.51 | C |
| ATOM | 1678 | CB | PHE | A | 242 | 17.589 | 62.944 | 26.285 | 1.00 | 45.41 | C |
| ATOM | 1679 | CG | PHE | A | 242 | 17.145 | 61.617 | 25.735 | 1.00 | 43.12 | C |
| ATOM | 1680 | CD1 | PHE | A | 242 | 16.885 | 60.545 | 26.578 | 1.00 | 42.42 | C |
| ATOM | 1681 | CE1 | PHE | A | 242 | 16.496 | 59.313 | 26.068 | 1.00 | 41.08 | C |
| ATOM | 1682 | CZ | PHE | A | 242 | 16.367 | 59.148 | 24.676 | 1.00 | 43.10 | C |
| ATOM | 1683 | CE2 | PHE | A | 242 | 16.618 | 60.222 | 23.824 | 1.00 | 40.87 | C |
| ATOM | 1684 | CD2 | PHE | A | 242 | 17.012 | 61.438 | 24.350 | 1.00 | 42.39 | C |
| ATOM | 1685 | C | PHE | A | 242 | 17.104 | 64.973 | 27.639 | 1.00 | 48.94 | C |
| ATOM | 1686 | O | PHE | A | 242 | 16.783 | 65.913 | 26.903 | 1.00 | 48.57 | O |
| ATOM | 1687 | N | GLU | A | 243 | 17.884 | 65.112 | 28.714 | 1.00 | 52.57 | N |
| ATOM | 1688 | CA | GLU | A | 243 | 18.514 | 66.391 | 29.046 | 1.00 | 55.94 | C |
| ATOM | 1689 | CB | GLU | A | 243 | 18.204 | 66.793 | 30.496 | 1.00 | 57.25 | C |
| ATOM | 1690 | CG | GLU | A | 243 | 16.930 | 67.634 | 30.664 | 1.00 | 62.51 | C |
| ATOM | 1691 | CD | GLU | A | 243 | 16.911 | 68.912 | 29.814 | 1.00 | 68.14 | C |
| ATOM | 1692 | OE1 | GLU | A | 243 | 17.901 | 69.697 | 29.854 | 1.00 | 69.83 | O |
| ATOM | 1693 | OE2 | GLU | A | 243 | 15.894 | 69.140 | 29.104 | 1.00 | 69.55 | O |
| ATOM | 1694 | C | GLU | A | 243 | 20.022 | 66.364 | 28.813 | 1.00 | 56.70 | C |
| ATOM | 1695 | O | GLU | A | 243 | 20.596 | 67.329 | 28.291 | 1.00 | 57.61 | O |
| ATOM | 1696 | N | HIS | A | 244 | 20.654 | 65.250 | 29.169 | 1.00 | 57.00 | N |
| ATOM | 1697 | CA | HIS | A | 244 | 22.111 | 65.145 | 29.128 | 1.00 | 57.57 | C |
| ATOM | 1698 | CB | HIS | A | 244 | 22.634 | 64.653 | 30.484 | 1.00 | 57.93 | C |
| ATOM | 1699 | CG | HIS | A | 244 | 22.177 | 65.491 | 31.641 | 1.00 | 60.15 | C |
| ATOM | 1700 | ND1 | HIS | A | 244 | 21.243 | 65.040 | 32.563 | 1.00 | 61.46 | N |
| ATOM | 1701 | CE1 | HIS | A | 244 | 21.021 | 65.986 | 33.459 | 1.00 | 61.53 | C |
| ATOM | 1702 | NE2 | HIS | A | 244 | 21.772 | 67.040 | 33.145 | 1.00 | 61.93 | N |
| ATOM | 1703 | CD2 | HIS | A | 244 | 22.501 | 66.761 | 32.008 | 1.00 | 60.89 | C |
| ATOM | 1704 | C | HIS | A | 244 | 22.632 | 64.251 | 27.999 | 1.00 | 57.12 | C |
| ATOM | 1705 | O | HIS | A | 244 | 21.946 | 63.321 | 27.564 | 1.00 | 56.42 | O |
| ATOM | 1706 | N | ASP | A | 245 | 23.850 | 64.550 | 27.542 | 1.00 | 56.65 | N |
| ATOM | 1707 | CA | ASP | A | 245 | 24.536 | 63.778 | 26.508 | 1.00 | 56.51 | C |
| ATOM | 1708 | CB | ASP | A | 245 | 25.982 | 64.254 | 26.364 | 1.00 | 56.84 | C |
| ATOM | 1709 | CG | ASP | A | 245 | 26.093 | 65.551 | 25.602 | 1.00 | 58.28 | C |
| ATOM | 1710 | OD1 | ASP | A | 245 | 25.109 | 66.322 | 25.555 | 1.00 | 60.57 | O |
| ATOM | 1711 | OD2 | ASP | A | 245 | 27.132 | 65.889 | 25.003 | 1.00 | 61.68 | O |
| ATOM | 1712 | C | ASP | A | 245 | 24.520 | 62.289 | 26.792 | 1.00 | 55.85 | C |
| ATOM | 1713 | O | ASP | A | 245 | 24.240 | 61.487 | 25.902 | 1.00 | 55.84 | O |
| ATOM | 1714 | N | GLU | A | 246 | 24.807 | 61.926 | 28.038 | 1.00 | 55.12 | N |
| ATOM | 1715 | CA | GLU | A | 246 | 24.814 | 60.528 | 28.473 | 1.00 | 54.57 | C |
| ATOM | 1716 | CB | GLU | A | 246 | 25.227 | 60.414 | 29.948 | 1.00 | 55.49 | C |
| ATOM | 1717 | CG | GLU | A | 246 | 26.247 | 61.439 | 30.419 | 1.00 | 59.92 | C |
| ATOM | 1718 | CD | GLU | A | 246 | 25.601 | 62.745 | 30.853 | 1.00 | 64.57 | C |
| ATOM | 1719 | OE1 | GLU | A | 246 | 24.864 | 62.732 | 31.873 | 1.00 | 66.43 | O |
| ATOM | 1720 | OE2 | GLU | A | 246 | 25.824 | 63.779 | 30.165 | 1.00 | 66.00 | O |
| ATOM | 1721 | C | GLU | A | 246 | 23.464 | 59.838 | 28.284 | 1.00 | 52.76 | C |
| ATOM | 1722 | O | GLU | A | 246 | 23.405 | 58.637 | 27.998 | 1.00 | 51.94 | O |
| ATOM | 1723 | N | GLU | A | 247 | 22.381 | 60.583 | 28.491 | 1.00 | 51.08 | N |
| ATOM | 1724 | CA | GLU | A | 247 | 21.037 | 60.031 | 28.289 | 1.00 | 50.00 | C |
| ATOM | 1725 | CB | GLU | A | 247 | 19.982 | 60.949 | 28.888 | 1.00 | 50.91 | C |
| ATOM | 1726 | CG | GLU | A | 247 | 20.048 | 61.069 | 30.398 | 1.00 | 54.76 | C |
| ATOM | 1727 | CD | GLU | A | 247 | 19.070 | 62.089 | 30.919 | 1.00 | 59.14 | C |
| ATOM | 1728 | OE1 | GLU | A | 247 | 19.189 | 63.281 | 30.568 | 1.00 | 61.88 | O |
| ATOM | 1729 | OE2 | GLU | A | 247 | 18.172 | 61.693 | 31.672 | 1.00 | 63.68 | O |
| ATOM | 1730 | C | GLU | A | 247 | 20.734 | 59.785 | 26.810 | 1.00 | 47.56 | C |
| ATOM | 1731 | O | GLU | A | 247 | 20.177 | 58.757 | 26.463 | 1.00 | 46.72 | O |
| ATOM | 1732 | N | ILE | A | 248 | 21.102 | 60.738 | 25.957 | 1.00 | 46.28 | N |
| ATOM | 1733 | CA | ILE | A | 248 | 20.964 | 60.598 | 24.498 | 1.00 | 46.12 | C |
| ATOM | 1734 | CB | ILE | A | 248 | 21.446 | 61.876 | 23.754 | 1.00 | 45.90 | C |
| ATOM | 1735 | CG1 | ILE | A | 248 | 20.599 | 63.092 | 24.141 | 1.00 | 44.91 | C |
| ATOM | 1736 | CD1 | ILE | A | 248 | 21.110 | 64.419 | 23.588 | 1.00 | 44.29 | C |
| ATOM | 1737 | CG2 | ILE | A | 248 | 21.444 | 61.658 | 22.233 | 1.00 | 45.48 | C |
| ATOM | 1738 | C | ILE | A | 248 | 21.741 | 59.390 | 23.988 | 1.00 | 46.47 | C |
| ATOM | 1739 | O | ILE | A | 248 | 21.221 | 58.613 | 23.199 | 1.00 | 46.50 | O |
| ATOM | 1740 | N | ILE | A | 249 | 22.977 | 59.223 | 24.462 | 1.00 | 46.70 | N |
| ATOM | 1741 | CA | ILE | A | 249 | 23.845 | 58.119 | 24.021 | 1.00 | 47.73 | C |
| ATOM | 1742 | CB | ILE | A | 249 | 25.315 | 58.342 | 24.516 | 1.00 | 48.27 | C |
| ATOM | 1743 | CG1 | ILE | A | 249 | 25.882 | 59.634 | 23.929 | 1.00 | 50.01 | C |
| ATOM | 1744 | CD1 | ILE | A | 249 | 27.162 | 60.114 | 24.638 | 1.00 | 54.07 | C |
| ATOM | 1745 | CG2 | ILE | A | 249 | 26.208 | 57.167 | 24.127 | 1.00 | 49.80 | C |
| ATOM | 1746 | C | ILE | A | 249 | 23.344 | 56.752 | 24.463 | 1.00 | 47.21 | C |
| ATOM | 1747 | O | ILE | A | 249 | 23.473 | 55.754 | 23.735 | 1.00 | 47.14 | O |
| ATOM | 1748 | N | ARG | A | 250 | 22.798 | 56.697 | 25.671 | 1.00 | 46.59 | N |
| ATOM | 1749 | CA | ARG | A | 250 | 22.259 | 55.454 | 26.197 | 1.00 | 46.70 | C |
| ATOM | 1750 | CB | ARG | A | 250 | 22.052 | 55.573 | 27.712 | 1.00 | 46.73 | C |
| ATOM | 1751 | CG | ARG | A | 250 | 21.612 | 54.297 | 28.415 | 1.00 | 47.47 | C |
| ATOM | 1752 | CD | ARG | A | 250 | 21.702 | 54.415 | 29.942 | 1.00 | 49.11 | C |
| ATOM | 1753 | NE | ARG | A | 250 | 21.290 | 53.191 | 30.631 | 1.00 | 50.87 | N |
| ATOM | 1754 | CZ | ARG | A | 250 | 20.217 | 53.076 | 31.429 | 1.00 | 50.66 | C |
| ATOM | 1755 | NH1 | ARG | A | 250 | 19.412 | 54.117 | 31.656 | 1.00 | 46.65 | N |
| ATOM | 1756 | NH2 | ARG | A | 250 | 19.955 | 51.909 | 32.006 | 1.00 | 50.26 | N |
| ATOM | 1757 | C | ARG | A | 250 | 20.949 | 55.097 | 25.483 | 1.00 | 46.42 | C |
| ATOM | 1758 | O | ARG | A | 250 | 20.617 | 53.922 | 25.352 | 1.00 | 47.00 | O |
| ATOM | 1759 | N | GLY | A | 251 | 20.224 | 56.113 | 25.018 | 1.00 | 46.84 | N |
| ATOM | 1760 | CA | GLY | A | 251 | 18.982 | 55.936 | 24.269 | 1.00 | 47.26 | C |
| ATOM | 1761 | C | GLY | A | 251 | 17.855 | 55.180 | 24.968 | 1.00 | 47.36 | C |
| ATOM | 1762 | O | GLY | A | 251 | 16.936 | 54.702 | 24.318 | 1.00 | 47.71 | O |
| ATOM | 1763 | N | GLN | A | 252 | 17.921 | 55.067 | 26.290 | 1.00 | 46.77 | N |
| ATOM | 1764 | CA | GLN | A | 252 | 16.872 | 54.400 | 27.058 | 1.00 | 46.61 | C |
| ATOM | 1765 | CB | GLN | A | 252 | 17.438 | 53.965 | 28.410 | 1.00 | 47.77 | C |
| ATOM | 1766 | CG | GLN | A | 252 | 16.745 | 52.797 | 29.034 | 1.00 | 53.27 | C |
| ATOM | 1767 | CD | GLN | A | 252 | 17.362 | 51.495 | 28.593 | 1.00 | 58.82 | C |
| ATOM | 1768 | OE1 | GLN | A | 252 | 16.922 | 50.902 | 27.587 | 1.00 | 62.58 | O |
| ATOM | 1769 | NE2 | GLN | A | 252 | 18.381 | 51.040 | 29.328 | 1.00 | 59.71 | N |
| ATOM | 1770 | C | GLN | A | 252 | 15.720 | 55.388 | 27.264 | 1.00 | 44.36 | C |
| ATOM | 1771 | O | GLN | A | 252 | 15.914 | 56.458 | 27.842 | 1.00 | 43.90 | O |
| ATOM | 1772 | N | VAL | A | 253 | 14.534 | 55.036 | 26.789 | 1.00 | 42.26 | N |
| ATOM | 1773 | CA | VAL | A | 253 | 13.366 | 55.917 | 26.887 | 1.00 | 41.30 | C |
| ATOM | 1774 | CB | VAL | A | 253 | 12.515 | 55.900 | 25.574 | 1.00 | 40.85 | C |
| ATOM | 1775 | CG1 | VAL | A | 253 | 11.398 | 56.917 | 25.657 | 1.00 | 41.13 | C |
| ATOM | 1776 | CG2 | VAL | A | 253 | 13.386 | 56.184 | 24.330 | 1.00 | 41.05 | C |
| ATOM | 1777 | C | VAL | A | 253 | 12.452 | 55.558 | 28.080 | 1.00 | 40.21 | C |
| ATOM | 1778 | O | VAL | A | 253 | 11.870 | 54.475 | 28.128 | 1.00 | 39.18 | O |
| ATOM | 1779 | N | PHE | A | 254 | 12.312 | 56.494 | 29.004 | 1.00 | 40.53 | N |
| ATOM | 1780 | CA | PHE | A | 254 | 11.415 | 56.340 | 30.147 | 1.00 | 41.30 | C |
| ATOM | 1781 | CB | PHE | A | 254 | 12.125 | 56.749 | 31.446 | 1.00 | 42.46 | C |
| ATOM | 1782 | CG | PHE | A | 254 | 11.181 | 56.979 | 32.597 | 1.00 | 45.89 | C |
| ATOM | 1783 | CD1 | PHE | A | 254 | 10.698 | 55.890 | 33.354 | 1.00 | 48.15 | C |
| ATOM | 1784 | CE1 | PHE | A | 254 | 9.794 | 56.087 | 34.453 | 1.00 | 46.65 | C |
| ATOM | 1785 | CZ | PHE | A | 254 | 9.368 | 57.391 | 34.762 | 1.00 | 47.61 | C |
| ATOM | 1786 | CE2 | PHE | A | 254 | 9.840 | 58.499 | 33.990 | 1.00 | 48.28 | C |
| ATOM | 1787 | CD2 | PHE | A | 254 | 10.742 | 58.287 | 32.922 | 1.00 | 47.87 | C |
| ATOM | 1788 | C | PHE | A | 254 | 10.192 | 57.216 | 29.960 | 1.00 | 40.77 | C |
| ATOM | 1789 | O | PHE | A | 254 | 10.324 | 58.388 | 29.630 | 1.00 | 40.62 | O |
| ATOM | 1790 | N | PHE | A | 255 | 9.011 | 56.656 | 30.202 | 1.00 | 39.76 | N |
| ATOM | 1791 | CA | PHE | A | 255 | 7.772 | 57.377 | 30.041 | 1.00 | 40.05 | C |
| ATOM | 1792 | CB | PHE | A | 255 | 6.744 | 56.512 | 29.293 | 1.00 | 38.87 | C |
| ATOM | 1793 | CG | PHE | A | 255 | 7.047 | 56.408 | 27.844 | 1.00 | 38.12 | C |
| ATOM | 1794 | CD1 | PHE | A | 255 | 6.520 | 57.332 | 26.945 | 1.00 | 37.51 | C |
| ATOM | 1795 | CE1 | PHE | A | 255 | 6.834 | 57.267 | 25.588 | 1.00 | 37.08 | C |
| ATOM | 1796 | CZ | PHE | A | 255 | 7.715 | 56.277 | 25.126 | 1.00 | 38.31 | C |
| ATOM | 1797 | CE2 | PHE | A | 255 | 8.251 | 55.353 | 26.034 | 1.00 | 37.42 | C |
| ATOM | 1798 | CD2 | PHE | A | 255 | 7.917 | 55.429 | 27.379 | 1.00 | 36.49 | C |
| ATOM | 1799 | C | PHE | A | 255 | 7.233 | 57.901 | 31.355 | 1.00 | 40.75 | C |
| ATOM | 1800 | O | PHE | A | 255 | 6.974 | 57.139 | 32.280 | 1.00 | 41.63 | O |
| ATOM | 1801 | N | ARG | A | 256 | 7.078 | 59.214 | 31.414 | 1.00 | 42.10 | N |
| ATOM | 1802 | CA | ARG | A | 256 | 6.613 | 59.911 | 32.614 | 1.00 | 43.68 | C |
| ATOM | 1803 | CB | ARG | A | 256 | 7.284 | 61.291 | 32.722 | 1.00 | 44.33 | C |
| ATOM | 1804 | CG | ARG | A | 256 | 7.050 | 62.233 | 31.549 | 1.00 | 46.48 | C |
| ATOM | 1805 | CD | ARG | A | 256 | 7.915 | 63.508 | 31.606 | 1.00 | 49.15 | C |
| ATOM | 1806 | NE | ARG | A | 256 | 9.248 | 63.277 | 31.034 | 1.00 | 53.26 | N |
| ATOM | 1807 | CZ | ARG | A | 256 | 10.334 | 64.018 | 31.293 | 1.00 | 54.62 | C |
| ATOM | 1808 | NH1 | ARG | A | 256 | 10.260 | 65.060 | 32.133 | 1.00 | 55.57 | N |
| ATOM | 1809 | NH2 | ARG | A | 256 | 11.502 | 63.720 | 30.716 | 1.00 | 52.61 | N |
| ATOM | 1810 | C | ARG | A | 256 | 5.096 | 60.051 | 32.658 | 1.00 | 43.59 | C |
| ATOM | 1811 | O | ARG | A | 256 | 4.525 | 60.251 | 33.724 | 1.00 | 44.72 | O |
| ATOM | 1812 | N | GLN | A | 257 | 4.454 | 59.930 | 31.498 | 1.00 | 42.63 | N |
| ATOM | 1813 | CA | GLN | A | 257 | 3.001 | 59.949 | 31.403 | 1.00 | 41.06 | C |
| ATOM | 1814 | CB | GLN | A | 257 | 2.538 | 61.061 | 30.445 | 1.00 | 42.46 | C |
| ATOM | 1815 | CG | GLN | A | 257 | 2.890 | 62.451 | 30.901 | 1.00 | 46.35 | C |
| ATOM | 1816 | CD | GLN | A | 257 | 1.908 | 62.984 | 31.916 | 1.00 | 51.18 | C |
| ATOM | 1817 | OE1 | GLN | A | 257 | 0.693 | 62.917 | 31.711 | 1.00 | 52.75 | O |
| ATOM | 1818 | NE2 | GLN | A | 257 | 2.428 | 63.504 | 33.020 | 1.00 | 55.05 | N |
| ATOM | 1819 | C | GLN | A | 257 | 2.510 | 58.618 | 30.872 | 1.00 | 38.68 | C |
| ATOM | 1820 | O | GLN | A | 257 | 3.267 | 57.849 | 30.300 | 1.00 | 38.45 | O |
| ATOM | 1821 | N | ARG | A | 258 | 1.226 | 58.353 | 31.047 | 1.00 | 36.14 | N |
| ATOM | 1822 | CA | ARG | A | 258 | 0.614 | 57.177 | 30.479 | 1.00 | 36.06 | C |
| ATOM | 1823 | CB | ARG | A | 258 | −0.820 | 57.048 | 30.997 | 1.00 | 34.77 | C |
| ATOM | 1824 | CG | ARG | A | 258 | −1.402 | 55.659 | 30.847 | 1.00 | 39.04 | C |
| ATOM | 1825 | CD | ARG | A | 258 | −1.624 | 55.230 | 29.442 | 1.00 | 40.99 | C |
| ATOM | 1826 | NE | ARG | A | 258 | −1.799 | 53.789 | 29.300 | 1.00 | 40.39 | N |
| ATOM | 1827 | CZ | ARG | A | 258 | −2.327 | 53.219 | 28.215 | 1.00 | 43.89 | C |
| ATOM | 1828 | NH1 | ARG | A | 258 | −2.730 | 53.966 | 27.158 | 1.00 | 45.06 | N |
| ATOM | 1829 | NH2 | ARG | A | 258 | −2.444 | 51.899 | 28.162 | 1.00 | 40.81 | N |
| ATOM | 1830 | C | ARG | A | 258 | 0.599 | 57.345 | 28.950 | 1.00 | 35.62 | C |
| ATOM | 1831 | O | ARG | A | 258 | 0.071 | 58.325 | 28.463 | 1.00 | 35.32 | O |
| ATOM | 1832 | N | VAL | A | 259 | 1.159 | 56.385 | 28.221 | 1.00 | 34.94 | N |
| ATOM | 1833 | CA | VAL | A | 259 | 1.223 | 56.440 | 26.755 | 1.00 | 34.87 | C |
| ATOM | 1834 | CB | VAL | A | 259 | 2.629 | 56.926 | 26.277 | 1.00 | 35.54 | C |
| ATOM | 1835 | CG1 | VAL | A | 259 | 2.782 | 56.824 | 24.747 | 1.00 | 34.21 | C |
| ATOM | 1836 | CG2 | VAL | A | 259 | 2.902 | 58.365 | 26.752 | 1.00 | 33.65 | C |
| ATOM | 1837 | C | VAL | A | 259 | 0.967 | 55.033 | 26.235 | 1.00 | 35.77 | C |
| ATOM | 1838 | O | VAL | A | 259 | 1.579 | 54.062 | 26.728 | 1.00 | 35.74 | O |
| ATOM | 1839 | N | SER | A | 260 | 0.055 | 54.901 | 25.267 | 1.00 | 35.24 | N |
| ATOM | 1840 | CA | SER | A | 260 | −0.269 | 53.601 | 24.698 | 1.00 | 36.49 | C |
| ATOM | 1841 | CB | SER | A | 260 | −1.247 | 53.749 | 23.525 | 1.00 | 36.47 | C |
| ATOM | 1842 | OG | SER | A | 260 | −0.608 | 54.285 | 22.377 | 1.00 | 37.04 | O |
| ATOM | 1843 | C | SER | A | 260 | 0.973 | 52.855 | 24.226 | 1.00 | 37.15 | C |
| ATOM | 1844 | O | SER | A | 260 | 1.981 | 53.465 | 23.874 | 1.00 | 37.30 | O |
| ATOM | 1845 | N | SER | A | 261 | 0.876 | 51.533 | 24.178 | 1.00 | 37.42 | N |
| ATOM | 1846 | CA | SER | A | 261 | 2.000 | 50.701 | 23.767 | 1.00 | 37.73 | C |
| ATOM | 1847 | CB | SER | A | 261 | 1.659 | 49.225 | 23.941 | 1.00 | 38.27 | C |
| ATOM | 1848 | OG | SER | A | 261 | 1.475 | 48.939 | 25.316 | 1.00 | 42.42 | O |
| ATOM | 1849 | C | SER | A | 261 | 2.399 | 50.965 | 22.325 | 1.00 | 37.68 | C |
| ATOM | 1850 | O | SER | A | 261 | 3.578 | 50.914 | 21.997 | 1.00 | 36.60 | O |
| ATOM | 1851 | N | GLU | A | 262 | 1.413 | 51.260 | 21.478 | 1.00 | 37.69 | N |
| ATOM | 1852 | CA | GLU | A | 262 | 1.662 | 51.578 | 20.080 | 1.00 | 38.33 | C |
| ATOM | 1853 | CB | GLU | A | 262 | 0.343 | 51.655 | 19.307 | 1.00 | 40.07 | C |
| ATOM | 1854 | CG | GLU | A | 262 | −0.522 | 50.401 | 19.444 | 1.00 | 47.46 | C |
| ATOM | 1855 | CD | GLU | A | 262 | −1.138 | 49.954 | 18.125 | 1.00 | 55.14 | C |
| ATOM | 1856 | OE1 | GLU | A | 262 | −1.716 | 50.811 | 17.407 | 1.00 | 58.98 | O |
| ATOM | 1857 | OE2 | GLU | A | 262 | −1.058 | 48.740 | 17.799 | 1.00 | 59.70 | O |
| ATOM | 1858 | C | GLU | A | 262 | 2.469 | 52.878 | 19.945 | 1.00 | 36.65 | C |
| ATOM | 1859 | O | GLU | A | 262 | 3.442 | 52.911 | 19.227 | 1.00 | 36.27 | O |
| ATOM | 1860 | N | CYS | A | 263 | 2.073 | 53.931 | 20.651 | 1.00 | 35.34 | N |
| ATOM | 1861 | CA | CYS | A | 263 | 2.822 | 55.188 | 20.621 | 1.00 | 34.46 | C |
| ATOM | 1862 | CB | CYS | A | 263 | 2.051 | 56.272 | 21.363 | 1.00 | 34.30 | C |
| ATOM | 1863 | SG | CYS | A | 263 | 2.728 | 57.931 | 21.207 | 1.00 | 34.38 | S |
| ATOM | 1864 | C | CYS | A | 263 | 4.250 | 55.021 | 21.181 | 1.00 | 34.46 | C |
| ATOM | 1865 | O | CYS | A | 263 | 5.221 | 55.477 | 20.556 | 1.00 | 32.45 | O |
| ATOM | 1866 | N | GLN | A | 264 | 4.385 | 54.325 | 22.321 | 1.00 | 33.42 | N |
| ATOM | 1867 | CA | GLN | A | 264 | 5.715 | 54.008 | 22.859 | 1.00 | 33.11 | C |
| ATOM | 1868 | CB | GLN | A | 264 | 5.629 | 53.110 | 24.097 | 1.00 | 33.38 | C |
| ATOM | 1869 | CG | GLN | A | 264 | 5.022 | 53.781 | 25.364 | 1.00 | 35.44 | C |
| ATOM | 1870 | CD | GLN | A | 264 | 5.296 | 52.968 | 26.647 | 1.00 | 37.59 | C |
| ATOM | 1871 | OE1 | GLN | A | 264 | 6.162 | 52.098 | 26.655 | 1.00 | 39.41 | O |
| ATOM | 1872 | NE2 | GLN | A | 264 | 4.566 | 53.262 | 27.717 | 1.00 | 33.63 | N |
| ATOM | 1873 | C | GLN | A | 264 | 6.578 | 53.314 | 21.795 | 1.00 | 33.15 | C |
| ATOM | 1874 | O | GLN | A | 264 | 7.753 | 53.689 | 21.606 | 1.00 | 32.43 | O |
| ATOM | 1875 | N | HIS | A | 265 | 6.001 | 52.322 | 21.110 | 1.00 | 32.53 | N |
| ATOM | 1876 | CA | HIS | A | 265 | 6.710 | 51.575 | 20.068 | 1.00 | 34.99 | C |
| ATOM | 1877 | CB | HIS | A | 265 | 5.836 | 50.455 | 19.469 | 1.00 | 36.23 | C |
| ATOM | 1878 | CG | HIS | A | 265 | 6.515 | 49.687 | 18.369 | 1.00 | 39.71 | C |
| ATOM | 1879 | ND1 | HIS | A | 265 | 6.481 | 50.086 | 17.050 | 1.00 | 40.80 | N |
| ATOM | 1880 | CE1 | HIS | A | 265 | 7.189 | 49.244 | 16.314 | 1.00 | 42.05 | C |
| ATOM | 1881 | NE2 | HIS | A | 265 | 7.687 | 48.312 | 17.110 | 1.00 | 42.46 | N |
| ATOM | 1882 | CD2 | HIS | A | 265 | 7.286 | 48.570 | 18.402 | 1.00 | 42.81 | C |
| ATOM | 1883 | C | HIS | A | 265 | 7.226 | 52.508 | 18.968 | 1.00 | 34.14 | C |
| ATOM | 1884 | O | HIS | A | 265 | 8.410 | 52.463 | 18.613 | 1.00 | 34.55 | O |
| ATOM | 1885 | N | LEU | A | 266 | 6.355 | 53.372 | 18.445 | 1.00 | 32.99 | N |
| ATOM | 1886 | CA | LEU | A | 266 | 6.778 | 54.306 | 17.394 | 1.00 | 32.41 | C |
| ATOM | 1887 | CB | LEU | A | 266 | 5.587 | 55.147 | 16.896 | 1.00 | 32.45 | C |
| ATOM | 1888 | CG | LEU | A | 266 | 5.863 | 56.209 | 15.818 | 1.00 | 33.18 | C |
| ATOM | 1889 | CD1 | LEU | A | 266 | 6.584 | 55.619 | 14.605 | 1.00 | 30.32 | C |
| ATOM | 1890 | CD2 | LEU | A | 266 | 4.511 | 56.820 | 15.367 | 1.00 | 30.03 | C |
| ATOM | 1891 | C | LEU | A | 266 | 7.885 | 55.211 | 17.904 | 1.00 | 31.79 | C |
| ATOM | 1892 | O | LEU | A | 266 | 8.907 | 55.417 | 17.231 | 1.00 | 31.14 | O |
| ATOM | 1893 | N | ILE | A | 267 | 7.706 | 55.750 | 19.112 | 1.00 | 31.36 | N |
| ATOM | 1894 | CA | ILE | A | 267 | 8.702 | 56.661 | 19.666 | 1.00 | 30.41 | C |
| ATOM | 1895 | CB | ILE | A | 267 | 8.273 | 57.184 | 21.052 | 1.00 | 29.95 | C |
| ATOM | 1896 | CG1 | ILE | A | 267 | 7.134 | 58.210 | 20.924 | 1.00 | 30.35 | C |
| ATOM | 1897 | CD1 | ILE | A | 267 | 6.410 | 58.513 | 22.271 | 1.00 | 30.22 | C |
| ATOM | 1898 | CG2 | ILE | A | 267 | 9.472 | 57.849 | 21.751 | 1.00 | 28.77 | C |
| ATOM | 1899 | C | ILE | A | 267 | 10.052 | 55.956 | 19.782 | 1.00 | 31.85 | C |
| ATOM | 1900 | O | ILE | A | 267 | 11.093 | 56.485 | 19.340 | 1.00 | 32.22 | O |
| ATOM | 1901 | N | ARG | A | 268 | 10.034 | 54.774 | 20.388 | 1.00 | 32.17 | N |
| ATOM | 1902 | CA | ARG | A | 268 | 11.248 | 53.988 | 20.594 | 1.00 | 34.65 | C |
| ATOM | 1903 | CB | ARG | A | 268 | 10.927 | 52.713 | 21.369 | 1.00 | 35.11 | C |
| ATOM | 1904 | CG | ARG | A | 268 | 10.707 | 52.931 | 22.864 | 1.00 | 39.57 | C |
| ATOM | 1905 | CD | ARG | A | 268 | 10.398 | 51.637 | 23.600 | 1.00 | 45.10 | C |
| ATOM | 1906 | NE | ARG | A | 268 | 9.725 | 51.866 | 24.890 | 1.00 | 48.08 | N |
| ATOM | 1907 | CZ | ARG | A | 268 | 10.370 | 52.338 | 25.935 | 1.00 | 49.97 | C |
| ATOM | 1908 | NH1 | ARG | A | 268 | 11.663 | 52.609 | 25.806 | 1.00 | 53.48 | N |
| ATOM | 1909 | NH2 | ARG | A | 268 | 9.753 | 52.551 | 27.093 | 1.00 | 48.54 | N |
| ATOM | 1910 | C | ARG | A | 268 | 11.921 | 53.622 | 19.278 | 1.00 | 34.22 | C |
| ATOM | 1911 | O | ARG | A | 268 | 13.140 | 53.491 | 19.223 | 1.00 | 34.73 | O |
| ATOM | 1912 | N | TRP | A | 269 | 11.124 | 53.464 | 18.225 | 1.00 | 34.43 | N |
| ATOM | 1913 | CA | TRP | A | 269 | 11.649 | 53.135 | 16.889 | 1.00 | 34.10 | C |
| ATOM | 1914 | CB | TRP | A | 269 | 10.503 | 52.657 | 15.992 | 1.00 | 34.69 | C |
| ATOM | 1915 | CG | TRP | A | 269 | 10.921 | 52.008 | 14.716 | 1.00 | 36.80 | C |
| ATOM | 1916 | CD1 | TRP | A | 269 | 12.191 | 51.632 | 14.352 | 1.00 | 39.30 | C |
| ATOM | 1917 | NE1 | TRP | A | 269 | 12.182 | 51.081 | 13.090 | 1.00 | 38.76 | N |
| ATOM | 1918 | CE2 | TRP | A | 269 | 10.895 | 51.071 | 12.618 | 1.00 | 37.50 | C |
| ATOM | 1919 | CD2 | TRP | A | 269 | 10.074 | 51.669 | 13.609 | 1.00 | 36.90 | C |
| ATOM | 1920 | CE3 | TRP | A | 269 | 8.703 | 51.787 | 13.359 | 1.00 | 35.95 | C |
| ATOM | 1921 | CZ3 | TRP | A | 269 | 8.197 | 51.332 | 12.136 | 1.00 | 37.55 | C |
| ATOM | 1922 | CH2 | TRP | A | 269 | 9.047 | 50.765 | 11.172 | 1.00 | 36.64 | C |
| ATOM | 1923 | CZ2 | TRP | A | 269 | 10.392 | 50.618 | 11.401 | 1.00 | 36.32 | C |
| ATOM | 1924 | C | TRP | A | 269 | 12.346 | 54.351 | 16.279 | 1.00 | 34.12 | C |
| ATOM | 1925 | O | TRP | A | 269 | 13.461 | 54.248 | 15.766 | 1.00 | 34.65 | O |
| ATOM | 1926 | N | CYS | A | 270 | 11.704 | 55.514 | 16.347 | 1.00 | 33.89 | N |
| ATOM | 1927 | CA | CYS | A | 270 | 12.315 | 56.762 | 15.881 | 1.00 | 33.18 | C |
| ATOM | 1928 | CB | CYS | A | 270 | 11.364 | 57.958 | 16.030 | 1.00 | 32.73 | C |
| ATOM | 1929 | SG | CYS | A | 270 | 9.894 | 57.933 | 14.980 | 1.00 | 34.78 | S |
| ATOM | 1930 | C | CYS | A | 270 | 13.593 | 57.085 | 16.627 | 1.00 | 33.31 | C |
| ATOM | 1931 | O | CYS | A | 270 | 14.471 | 57.759 | 16.085 | 1.00 | 33.37 | O |
| ATOM | 1932 | N | LEU | A | 271 | 13.686 | 56.635 | 17.879 | 1.00 | 32.81 | N |
| ATOM | 1933 | CA | LEU | A | 271 | 14.835 | 56.934 | 18.711 | 1.00 | 33.61 | C |
| ATOM | 1934 | CB | LEU | A | 271 | 14.405 | 57.342 | 20.143 | 1.00 | 33.79 | C |
| ATOM | 1935 | CG | LEU | A | 271 | 13.573 | 58.649 | 20.223 | 1.00 | 33.39 | C |
| ATOM | 1936 | CD1 | LEU | A | 271 | 13.178 | 58.971 | 21.668 | 1.00 | 32.58 | C |
| ATOM | 1937 | CD2 | LEU | A | 271 | 14.330 | 59.820 | 19.602 | 1.00 | 29.30 | C |
| ATOM | 1938 | C | LEU | A | 271 | 15.805 | 55.766 | 18.761 | 1.00 | 34.83 | C |
| ATOM | 1939 | O | LEU | A | 271 | 16.536 | 55.613 | 19.727 | 1.00 | 34.16 | O |
| ATOM | 1940 | N | ALA | A | 272 | 15.836 | 54.958 | 17.705 | 1.00 | 35.68 | N |
| ATOM | 1941 | CA | ALA | A | 272 | 16.796 | 53.853 | 17.658 | 1.00 | 37.00 | C |
| ATOM | 1942 | CB | ALA | A | 272 | 16.563 | 52.994 | 16.429 | 1.00 | 37.89 | C |
| ATOM | 1943 | C | ALA | A | 272 | 18.191 | 54.460 | 17.658 | 1.00 | 37.13 | C |
| ATOM | 1944 | O | ALA | A | 272 | 18.436 | 55.466 | 16.996 | 1.00 | 36.83 | O |
| ATOM | 1945 | N | LEU | A | 273 | 19.087 | 53.886 | 18.447 | 1.00 | 38.00 | N |
| ATOM | 1946 | CA | LEU | A | 273 | 20.464 | 54.378 | 18.537 | 1.00 | 39.46 | C |
| ATOM | 1947 | CB | LEU | A | 273 | 21.266 | 53.531 | 19.532 | 1.00 | 39.79 | C |
| ATOM | 1948 | CG | LEU | A | 273 | 20.990 | 53.795 | 21.011 | 1.00 | 40.61 | C |
| ATOM | 1949 | CD1 | LEU | A | 273 | 21.923 | 52.966 | 21.914 | 1.00 | 40.75 | C |
| ATOM | 1950 | CD2 | LEU | A | 273 | 21.174 | 55.277 | 21.304 | 1.00 | 39.54 | C |
| ATOM | 1951 | C | LEU | A | 273 | 21.146 | 54.350 | 17.168 | 1.00 | 40.47 | C |
| ATOM | 1952 | O | LEU | A | 273 | 21.742 | 55.331 | 16.747 | 1.00 | 40.46 | O |
| ATOM | 1953 | N | ARG | A | 274 | 21.051 | 53.225 | 16.470 | 1.00 | 41.49 | N |
| ATOM | 1954 | CA | ARG | A | 274 | 21.670 | 53.138 | 15.151 | 1.00 | 43.63 | C |
| ATOM | 1955 | CB | ARG | A | 274 | 21.929 | 51.683 | 14.753 | 1.00 | 44.96 | C |
| ATOM | 1956 | CG | ARG | A | 274 | 22.917 | 50.943 | 15.665 | 1.00 | 51.46 | C |
| ATOM | 1957 | CD | ARG | A | 274 | 23.145 | 49.470 | 15.275 | 1.00 | 60.47 | C |
| ATOM | 1958 | NE | ARG | A | 274 | 23.426 | 49.354 | 13.842 | 1.00 | 66.38 | N |
| ATOM | 1959 | CZ | ARG | A | 274 | 23.511 | 48.212 | 13.172 | 1.00 | 69.93 | C |
| ATOM | 1960 | NH1 | ARG | A | 274 | 23.344 | 47.048 | 13.792 | 1.00 | 71.16 | N |
| ATOM | 1961 | NH2 | ARG | A | 274 | 23.775 | 48.239 | 11.868 | 1.00 | 72.00 | N |
| ATOM | 1962 | C | ARG | A | 274 | 20.784 | 53.826 | 14.117 | 1.00 | 42.18 | C |
| ATOM | 1963 | O | ARG | A | 274 | 19.632 | 53.469 | 13.959 | 1.00 | 42.54 | O |
| ATOM | 1964 | N | PRO | A | 275 | 21.325 | 54.807 | 13.409 | 1.00 | 41.48 | N |
| ATOM | 1965 | CA | PRO | A | 275 | 20.566 | 55.529 | 12.383 | 1.00 | 41.53 | C |
| ATOM | 1966 | CB | PRO | A | 275 | 21.655 | 56.302 | 11.648 | 1.00 | 41.70 | C |
| ATOM | 1967 | CG | PRO | A | 275 | 22.618 | 56.629 | 12.745 | 1.00 | 41.06 | C |
| ATOM | 1968 | CD | PRO | A | 275 | 22.693 | 55.340 | 13.546 | 1.00 | 41.06 | C |
| ATOM | 1969 | C | PRO | A | 275 | 19.784 | 54.624 | 11.429 | 1.00 | 41.71 | C |
| ATOM | 1970 | O | PRO | A | 275 | 18.633 | 54.932 | 11.132 | 1.00 | 39.61 | O |
| ATOM | 1971 | N | SER | A | 276 | 20.393 | 53.516 | 10.993 | 1.00 | 41.90 | N |
| ATOM | 1972 | CA | SER | A | 276 | 19.774 | 52.587 | 10.040 | 1.00 | 42.57 | C |
| ATOM | 1973 | CB | SER | A | 276 | 20.831 | 51.624 | 9.446 | 1.00 | 43.08 | C |
| ATOM | 1974 | OG | SER | A | 276 | 21.290 | 50.683 | 10.419 | 1.00 | 45.84 | O |
| ATOM | 1975 | C | SER | A | 276 | 18.597 | 51.799 | 10.613 | 1.00 | 41.74 | C |
| ATOM | 1976 | O | SER | A | 276 | 17.786 | 51.287 | 9.845 | 1.00 | 42.39 | O |
| ATOM | 1977 | N | ASP | A | 277 | 18.497 | 51.696 | 11.942 | 1.00 | 40.48 | N |
| ATOM | 1978 | CA | ASP | A | 277 | 17.344 | 51.038 | 12.575 | 1.00 | 39.45 | C |
| ATOM | 1979 | CB | ASP | A | 277 | 17.676 | 50.520 | 13.981 | 1.00 | 40.14 | C |
| ATOM | 1980 | CG | ASP | A | 277 | 18.671 | 49.374 | 13.974 | 1.00 | 41.45 | C |
| ATOM | 1981 | OD1 | ASP | A | 277 | 18.697 | 48.577 | 13.010 | 1.00 | 43.47 | O |
| ATOM | 1982 | OD2 | ASP | A | 277 | 19.471 | 49.221 | 14.915 | 1.00 | 43.49 | O |
| ATOM | 1983 | C | ASP | A | 277 | 16.102 | 51.946 | 12.676 | 1.00 | 38.59 | C |
| ATOM | 1984 | O | ASP | A | 277 | 15.010 | 51.486 | 13.014 | 1.00 | 37.61 | O |
| ATOM | 1985 | N | ARG | A | 278 | 16.269 | 53.227 | 12.364 | 1.00 | 37.09 | N |
| ATOM | 1986 | CA | ARG | A | 278 | 15.145 | 54.159 | 12.448 | 1.00 | 35.81 | C |
| ATOM | 1987 | CB | ARG | A | 278 | 15.657 | 55.598 | 12.545 | 1.00 | 34.54 | C |
| ATOM | 1988 | CG | ARG | A | 278 | 16.407 | 55.836 | 13.836 | 1.00 | 34.40 | C |
| ATOM | 1989 | CD | ARG | A | 278 | 17.017 | 57.225 | 13.957 | 1.00 | 35.33 | C |
| ATOM | 1990 | NE | ARG | A | 278 | 18.119 | 57.186 | 14.913 | 1.00 | 35.31 | N |
| ATOM | 1991 | CZ | ARG | A | 278 | 19.163 | 57.996 | 14.913 | 1.00 | 36.13 | C |
| ATOM | 1992 | NH1 | ARG | A | 278 | 19.286 | 58.971 | 14.010 | 1.00 | 34.75 | N |
| ATOM | 1993 | NH2 | ARG | A | 278 | 20.103 | 57.815 | 15.829 | 1.00 | 36.28 | N |
| ATOM | 1994 | C | ARG | A | 278 | 14.223 | 53.983 | 11.243 | 1.00 | 36.08 | C |
| ATOM | 1995 | O | ARG | A | 278 | 14.687 | 53.610 | 10.156 | 1.00 | 36.69 | O |
| ATOM | 1996 | N | PRO | A | 279 | 12.936 | 54.275 | 11.421 | 1.00 | 35.45 | N |
| ATOM | 1997 | CA | PRO | A | 279 | 11.984 | 54.193 | 10.314 | 1.00 | 35.56 | C |
| ATOM | 1998 | CB | PRO | A | 279 | 10.627 | 54.303 | 11.004 | 1.00 | 35.57 | C |
| ATOM | 1999 | CG | PRO | A | 279 | 10.915 | 55.147 | 12.224 | 1.00 | 35.53 | C |
| ATOM | 2000 | CD | PRO | A | 279 | 12.284 | 54.710 | 12.677 | 1.00 | 35.37 | C |
| ATOM | 2001 | C | PRO | A | 279 | 12.174 | 55.322 | 9.311 | 1.00 | 35.53 | C |
| ATOM | 2002 | O | PRO | A | 279 | 12.784 | 56.354 | 9.626 | 1.00 | 35.63 | O |
| ATOM | 2003 | N | THR | A | 280 | 11.661 | 55.107 | 8.101 | 1.00 | 34.84 | N |
| ATOM | 2004 | CA | THR | A | 280 | 11.618 | 56.145 | 7.079 | 1.00 | 34.43 | C |
| ATOM | 2005 | CB | THR | A | 280 | 11.509 | 55.513 | 5.683 | 1.00 | 34.63 | C |
| ATOM | 2006 | OG1 | THR | A | 280 | 10.344 | 54.690 | 5.655 | 1.00 | 34.43 | O |
| ATOM | 2007 | CG2 | THR | A | 280 | 12.712 | 54.555 | 5.379 | 1.00 | 35.88 | C |
| ATOM | 2008 | C | THR | A | 280 | 10.334 | 56.900 | 7.337 | 1.00 | 34.28 | C |
| ATOM | 2009 | O | THR | A | 280 | 9.501 | 56.458 | 8.120 | 1.00 | 33.22 | O |
| ATOM | 2010 | N | PHE | A | 281 | 10.129 | 58.012 | 6.637 | 1.00 | 35.30 | N |
| ATOM | 2011 | CA | PHE | A | 281 | 8.893 | 58.771 | 6.797 | 1.00 | 35.82 | C |
| ATOM | 2012 | CB | PHE | A | 281 | 8.892 | 60.020 | 5.907 | 1.00 | 36.90 | C |
| ATOM | 2013 | CG | PHE | A | 281 | 9.984 | 61.009 | 6.223 | 1.00 | 38.29 | C |
| ATOM | 2014 | CD1 | PHE | A | 281 | 10.332 | 61.300 | 7.536 | 1.00 | 39.19 | C |
| ATOM | 2015 | CE1 | PHE | A | 281 | 11.320 | 62.234 | 7.823 | 1.00 | 39.73 | C |
| ATOM | 2016 | CZ | PHE | A | 281 | 11.968 | 62.874 | 6.810 | 1.00 | 41.57 | C |
| ATOM | 2017 | CE2 | PHE | A | 281 | 11.621 | 62.608 | 5.483 | 1.00 | 43.04 | C |
| ATOM | 2018 | CD2 | PHE | A | 281 | 10.633 | 61.681 | 5.200 | 1.00 | 41.16 | C |
| ATOM | 2019 | C | PHE | A | 281 | 7.690 | 57.894 | 6.477 | 1.00 | 36.17 | C |
| ATOM | 2020 | O | PHE | A | 281 | 6.671 | 57.924 | 7.179 | 1.00 | 35.36 | O |
| ATOM | 2021 | N | GLU | A | 282 | 7.815 | 57.101 | 5.414 | 1.00 | 35.33 | N |
| ATOM | 2022 | CA | GLU | A | 282 | 6.741 | 56.194 | 4.992 | 1.00 | 35.04 | C |
| ATOM | 2023 | CB | GLU | A | 282 | 7.154 | 55.461 | 3.700 | 1.00 | 35.95 | C |
| ATOM | 2024 | CG | GLU | A | 282 | 6.092 | 54.530 | 3.141 | 1.00 | 38.88 | C |
| ATOM | 2025 | CD | GLU | A | 282 | 6.504 | 53.872 | 1.819 | 1.00 | 42.76 | C |
| ATOM | 2026 | OE1 | GLU | A | 282 | 7.654 | 54.056 | 1.362 | 1.00 | 43.67 | O |
| ATOM | 2027 | OE2 | GLU | A | 282 | 5.654 | 53.182 | 1.233 | 1.00 | 43.19 | O |
| ATOM | 2028 | C | GLU | A | 282 | 6.385 | 55.199 | 6.084 | 1.00 | 34.27 | C |
| ATOM | 2029 | O | GLU | A | 282 | 5.209 | 54.986 | 6.378 | 1.00 | 34.51 | O |
| ATOM | 2030 | N | GLU | A | 283 | 7.397 | 54.594 | 6.693 | 1.00 | 34.18 | N |
| ATOM | 2031 | CA | GLU | A | 283 | 7.194 | 53.640 | 7.795 | 1.00 | 34.58 | C |
| ATOM | 2032 | CB | GLU | A | 283 | 8.512 | 53.012 | 8.208 | 1.00 | 35.26 | C |
| ATOM | 2033 | CG | GLU | A | 283 | 9.077 | 52.096 | 7.131 | 1.00 | 38.60 | C |
| ATOM | 2034 | CD | GLU | A | 283 | 10.406 | 51.501 | 7.502 | 1.00 | 40.02 | C |
| ATOM | 2035 | OE1 | GLU | A | 283 | 11.340 | 52.257 | 7.832 | 1.00 | 41.52 | O |
| ATOM | 2036 | OE2 | GLU | A | 283 | 10.517 | 50.266 | 7.435 | 1.00 | 44.14 | O |
| ATOM | 2037 | C | GLU | A | 283 | 6.524 | 54.259 | 9.014 | 1.00 | 33.96 | C |
| ATOM | 2038 | O | GLU | A | 283 | 5.700 | 53.614 | 9.674 | 1.00 | 33.93 | O |
| ATOM | 2039 | N | ILE | A | 284 | 6.859 | 55.517 | 9.298 | 1.00 | 33.26 | N |
| ATOM | 2040 | CA | ILE | A | 284 | 6.204 | 56.233 | 10.401 | 1.00 | 31.63 | C |
| ATOM | 2041 | CB | ILE | A | 284 | 6.889 | 57.590 | 10.650 | 1.00 | 31.52 | C |
| ATOM | 2042 | CG1 | ILE | A | 284 | 8.282 | 57.373 | 11.252 | 1.00 | 29.17 | C |
| ATOM | 2043 | CD1 | ILE | A | 284 | 9.195 | 58.585 | 11.190 | 1.00 | 32.09 | C |
| ATOM | 2044 | CG2 | ILE | A | 284 | 6.002 | 58.489 | 11.602 | 1.00 | 29.23 | C |
| ATOM | 2045 | C | ILE | A | 284 | 4.732 | 56.430 | 10.089 | 1.00 | 31.80 | C |
| ATOM | 2046 | O | ILE | A | 284 | 3.856 | 56.165 | 10.917 | 1.00 | 31.97 | O |
| ATOM | 2047 | N | GLN | A | 285 | 4.451 | 56.917 | 8.886 | 1.00 | 32.64 | N |
| ATOM | 2048 | CA | GLN | A | 285 | 3.070 | 57.204 | 8.515 | 1.00 | 32.77 | C |
| ATOM | 2049 | CB | GLN | A | 285 | 3.022 | 58.099 | 7.280 | 1.00 | 32.86 | C |
| ATOM | 2050 | CG | GLN | A | 285 | 3.373 | 59.566 | 7.613 | 1.00 | 32.93 | C |
| ATOM | 2051 | CD | GLN | A | 285 | 3.056 | 60.507 | 6.485 | 1.00 | 35.30 | C |
| ATOM | 2052 | OE1 | GLN | A | 285 | 3.637 | 60.401 | 5.395 | 1.00 | 34.32 | O |
| ATOM | 2053 | NE2 | GLN | A | 285 | 2.122 | 61.423 | 6.725 | 1.00 | 33.69 | N |
| ATOM | 2054 | C | GLN | A | 285 | 2.239 | 55.948 | 8.334 | 1.00 | 33.74 | C |
| ATOM | 2055 | O | GLN | A | 285 | 1.021 | 55.996 | 8.454 | 1.00 | 34.52 | O |
| ATOM | 2056 | N | ASN | A | 286 | 2.889 | 54.816 | 8.084 | 1.00 | 34.59 | N |
| ATOM | 2057 | CA | ASN | A | 286 | 2.165 | 53.533 | 8.016 | 1.00 | 36.22 | C |
| ATOM | 2058 | CB | ASN | A | 286 | 2.770 | 52.607 | 6.966 | 1.00 | 35.90 | C |
| ATOM | 2059 | CG | ASN | A | 286 | 2.450 | 53.042 | 5.553 | 1.00 | 37.57 | C |
| ATOM | 2060 | OD1 | ASN | A | 286 | 1.397 | 53.611 | 5.283 | 1.00 | 37.74 | O |
| ATOM | 2061 | ND2 | ASN | A | 286 | 3.373 | 52.785 | 4.642 | 1.00 | 39.91 | N |
| ATOM | 2062 | C | ASN | A | 286 | 2.079 | 52.805 | 9.360 | 1.00 | 36.58 | C |
| ATOM | 2063 | O | ASN | A | 286 | 1.432 | 51.767 | 9.466 | 1.00 | 37.11 | O |
| ATOM | 2064 | N | HIS | A | 287 | 2.723 | 53.356 | 10.384 | 1.00 | 36.48 | N |
| ATOM | 2065 | CA | HIS | A | 287 | 2.677 | 52.771 | 11.717 | 1.00 | 36.00 | C |
| ATOM | 2066 | CB | HIS | A | 287 | 3.525 | 53.596 | 12.697 | 1.00 | 35.69 | C |
| ATOM | 2067 | CG | HIS | A | 287 | 3.703 | 52.938 | 14.029 | 1.00 | 33.97 | C |
| ATOM | 2068 | ND1 | HIS | A | 287 | 4.826 | 52.211 | 14.359 | 1.00 | 35.79 | N |
| ATOM | 2069 | CE1 | HIS | A | 287 | 4.706 | 51.749 | 15.592 | 1.00 | 34.10 | C |
| ATOM | 2070 | NE2 | HIS | A | 287 | 3.537 | 52.138 | 16.066 | 1.00 | 36.32 | N |
| ATOM | 2071 | CD2 | HIS | A | 287 | 2.888 | 52.875 | 15.103 | 1.00 | 33.02 | C |
| ATOM | 2072 | C | HIS | A | 287 | 1.238 | 52.724 | 12.223 | 1.00 | 36.98 | C |
| ATOM | 2073 | O | HIS | A | 287 | 0.475 | 53.663 | 11.985 | 1.00 | 36.67 | O |
| ATOM | 2074 | N | PRO | A | 288 | 0.870 | 51.638 | 12.909 | 1.00 | 37.63 | N |
| ATOM | 2075 | CA | PRO | A | 288 | −0.465 | 51.480 | 13.468 | 1.00 | 38.40 | C |
| ATOM | 2076 | CB | PRO | A | 288 | −0.318 | 50.203 | 14.315 | 1.00 | 39.46 | C |
| ATOM | 2077 | CG | PRO | A | 288 | 0.684 | 49.420 | 13.576 | 1.00 | 40.31 | C |
| ATOM | 2078 | CD | PRO | A | 288 | 1.699 | 50.447 | 13.174 | 1.00 | 38.01 | C |
| ATOM | 2079 | C | PRO | A | 288 | −0.936 | 52.652 | 14.325 | 1.00 | 37.78 | C |
| ATOM | 2080 | O | PRO | A | 288 | −2.096 | 53.024 | 14.227 | 1.00 | 38.92 | O |
| ATOM | 2081 | N | TRP | A | 289 | −0.062 | 53.231 | 15.143 | 1.00 | 37.99 | N |
| ATOM | 2082 | CA | TRP | A | 289 | −0.459 | 54.387 | 15.951 | 1.00 | 36.84 | C |
| ATOM | 2083 | CB | TRP | A | 289 | 0.642 | 54.779 | 16.932 | 1.00 | 37.43 | C |
| ATOM | 2084 | CG | TRP | A | 289 | 0.197 | 55.892 | 17.862 | 1.00 | 36.44 | C |
| ATOM | 2085 | CD1 | TRP | A | 289 | −0.601 | 55.776 | 18.969 | 1.00 | 36.79 | C |
| ATOM | 2086 | NE1 | TRP | A | 289 | −0.800 | 57.014 | 19.542 | 1.00 | 35.91 | N |
| ATOM | 2087 | CE2 | TRP | A | 289 | −0.136 | 57.956 | 18.795 | 1.00 | 35.04 | C |
| ATOM | 2088 | CD2 | TRP | A | 289 | 0.500 | 57.281 | 17.730 | 1.00 | 35.44 | C |
| ATOM | 2089 | CE3 | TRP | A | 289 | 1.275 | 58.034 | 16.822 | 1.00 | 34.75 | C |
| ATOM | 2090 | CZ3 | TRP | A | 289 | 1.365 | 59.412 | 17.000 | 1.00 | 33.10 | C |
| ATOM | 2091 | CH2 | TRP | A | 289 | 0.719 | 60.046 | 18.072 | 1.00 | 32.65 | C |
| ATOM | 2092 | CZ2 | TRP | A | 289 | −0.024 | 59.337 | 18.980 | 1.00 | 35.41 | C |
| ATOM | 2093 | C | TRP | A | 289 | −0.886 | 55.598 | 15.102 | 1.00 | 37.02 | C |
| ATOM | 2094 | O | TRP | A | 289 | −1.703 | 56.402 | 15.551 | 1.00 | 36.85 | O |
| ATOM | 2095 | N | MET | A | 290 | −0.375 | 55.704 | 13.875 | 1.00 | 37.67 | N |
| ATOM | 2096 | CA | MET | A | 290 | −0.681 | 56.857 | 13.002 | 1.00 | 39.50 | C |
| ATOM | 2097 | CB | MET | A | 290 | 0.475 | 57.119 | 12.038 | 1.00 | 38.69 | C |
| ATOM | 2098 | CG | MET | A | 290 | 1.770 | 57.552 | 12.737 | 1.00 | 39.97 | C |
| ATOM | 2099 | SD | MET | A | 290 | 2.026 | 59.340 | 12.660 | 1.00 | 43.39 | S |
| ATOM | 2100 | CE | MET | A | 290 | 0.826 | 59.826 | 13.609 | 1.00 | 36.57 | C |
| ATOM | 2101 | C | MET | A | 290 | −1.973 | 56.787 | 12.186 | 1.00 | 41.20 | C |
| ATOM | 2102 | O | MET | A | 290 | −2.269 | 57.703 | 11.397 | 1.00 | 40.58 | O |
| ATOM | 2103 | N | GLN | A | 291 | −2.735 | 55.709 | 12.338 | 1.00 | 43.28 | N |
| ATOM | 2104 | CA | GLN | A | 291 | −3.928 | 55.516 | 11.504 | 1.00 | 45.49 | C |
| ATOM | 2105 | CB | GLN | A | 291 | −4.294 | 54.031 | 11.420 | 1.00 | 46.70 | C |
| ATOM | 2106 | CG | GLN | A | 291 | −3.169 | 53.163 | 10.863 | 1.00 | 52.12 | C |
| ATOM | 2107 | CD | GLN | A | 291 | −2.989 | 53.257 | 9.330 | 1.00 | 58.53 | C |
| ATOM | 2108 | OE1 | GLN | A | 291 | −3.107 | 54.339 | 8.723 | 1.00 | 59.20 | O |
| ATOM | 2109 | NE2 | GLN | A | 291 | −2.674 | 52.113 | 8.708 | 1.00 | 62.36 | N |
| ATOM | 2110 | C | GLN | A | 291 | −5.112 | 56.329 | 12.001 | 1.00 | 45.68 | C |
| ATOM | 2111 | O | GLN | A | 291 | −5.165 | 56.708 | 13.177 | 1.00 | 45.87 | O |
| ATOM | 2112 | N | ASP | A | 292 | −6.064 | 56.590 | 11.100 | 1.00 | 46.07 | N |
| ATOM | 2113 | CA | ASP | A | 292 | −7.305 | 57.331 | 11.410 | 1.00 | 46.57 | C |
| ATOM | 2114 | CB | ASP | A | 292 | −8.195 | 56.556 | 12.391 | 1.00 | 47.72 | C |
| ATOM | 2115 | CG | ASP | A | 292 | −8.324 | 55.099 | 12.018 | 1.00 | 52.03 | C |
| ATOM | 2116 | OD1 | ASP | A | 292 | −8.714 | 54.836 | 10.858 | 1.00 | 54.96 | O |
| ATOM | 2117 | OD2 | ASP | A | 292 | −8.031 | 54.164 | 12.805 | 1.00 | 56.19 | O |
| ATOM | 2118 | C | ASP | A | 292 | −7.071 | 58.739 | 11.961 | 1.00 | 45.48 | C |
| ATOM | 2119 | O | ASP | A | 292 | −7.779 | 59.181 | 12.880 | 1.00 | 44.34 | O |
| ATOM | 2120 | N | VAL | A | 293 | −6.067 | 59.433 | 11.424 | 1.00 | 44.87 | N |
| ATOM | 2121 | CA | VAL | A | 293 | −5.750 | 60.787 | 11.882 | 1.00 | 44.61 | C |
| ATOM | 2122 | CB | VAL | A | 293 | −4.495 | 61.338 | 11.181 | 1.00 | 44.85 | C |
| ATOM | 2123 | CG1 | VAL | A | 293 | −4.791 | 61.662 | 9.735 | 1.00 | 44.77 | C |
| ATOM | 2124 | CG2 | VAL | A | 293 | −3.989 | 62.568 | 11.896 | 1.00 | 43.11 | C |
| ATOM | 2125 | C | VAL | A | 293 | −6.935 | 61.709 | 11.640 | 1.00 | 44.94 | C |
| ATOM | 2126 | O | VAL | A | 293 | −7.658 | 61.532 | 10.653 | 1.00 | 45.74 | O |
| ATOM | 2127 | N | LEU | A | 294 | −7.149 | 62.668 | 12.538 | 1.00 | 44.60 | N |
| ATOM | 2128 | CA | LEU | A | 294 | −8.209 | 63.650 | 12.354 | 1.00 | 44.83 | C |
| ATOM | 2129 | CB | LEU | A | 294 | −8.489 | 64.416 | 13.641 | 1.00 | 43.88 | C |
| ATOM | 2130 | CG | LEU | A | 294 | −9.009 | 63.721 | 14.886 | 1.00 | 44.35 | C |
| ATOM | 2131 | CD1 | LEU | A | 294 | −9.118 | 64.760 | 15.972 | 1.00 | 42.61 | C |
| ATOM | 2132 | CD2 | LEU | A | 294 | −10.337 | 63.036 | 14.632 | 1.00 | 45.06 | C |
| ATOM | 2133 | C | LEU | A | 294 | −7.763 | 64.655 | 11.312 | 1.00 | 45.35 | C |
| ATOM | 2134 | O | LEU | A | 294 | −6.570 | 64.888 | 11.142 | 1.00 | 45.48 | O |
| ATOM | 2135 | N | LEU | A | 295 | −8.728 | 65.266 | 10.629 | 1.00 | 46.10 | N |
| ATOM | 2136 | CA | LEU | A | 295 | −8.444 | 66.358 | 9.712 | 1.00 | 46.34 | C |
| ATOM | 2137 | CB | LEU | A | 295 | −9.645 | 66.586 | 8.790 | 1.00 | 47.42 | C |
| ATOM | 2138 | CG | LEU | A | 295 | −9.552 | 65.968 | 7.380 | 1.00 | 50.24 | C |
| ATOM | 2139 | CD1 | LEU | A | 295 | −9.352 | 64.460 | 7.415 | 1.00 | 51.66 | C |
| ATOM | 2140 | CD2 | LEU | A | 295 | −10.812 | 66.288 | 6.595 | 1.00 | 54.37 | C |
| ATOM | 2141 | C | LEU | A | 295 | −8.123 | 67.612 | 10.527 | 1.00 | 46.02 | C |
| ATOM | 2142 | O | LEU | A | 295 | −8.531 | 67.723 | 11.693 | 1.00 | 44.80 | O |
| ATOM | 2143 | N | PRO | A | 296 | −7.366 | 68.544 | 9.955 | 1.00 | 46.46 | N |
| ATOM | 2144 | CA | PRO | A | 296 | −7.048 | 69.790 | 10.658 | 1.00 | 47.09 | C |
| ATOM | 2145 | CB | PRO | A | 296 | −6.405 | 70.633 | 9.561 | 1.00 | 46.93 | C |
| ATOM | 2146 | CG | PRO | A | 296 | −5.698 | 69.609 | 8.741 | 1.00 | 45.84 | C |
| ATOM | 2147 | CD | PRO | A | 296 | −6.708 | 68.496 | 8.638 | 1.00 | 46.93 | C |
| ATOM | 2148 | C | PRO | A | 296 | −8.282 | 70.465 | 11.266 | 1.00 | 48.28 | C |
| ATOM | 2149 | O | PRO | A | 296 | −8.280 | 70.739 | 12.474 | 1.00 | 47.68 | O |
| ATOM | 2150 | N | GLN | A | 297 | −9.335 | 70.684 | 10.480 | 1.00 | 50.01 | N |
| ATOM | 2151 | CA | GLN | A | 297 | −10.537 | 71.328 | 11.022 | 1.00 | 51.78 | C |
| ATOM | 2152 | CB | GLN | A | 297 | −11.572 | 71.636 | 9.933 | 1.00 | 52.87 | C |
| ATOM | 2153 | CG | GLN | A | 297 | −12.552 | 72.781 | 10.298 | 1.00 | 55.96 | C |
| ATOM | 2154 | CD | GLN | A | 297 | −11.858 | 74.122 | 10.632 | 1.00 | 60.05 | C |
| ATOM | 2155 | OE1 | GLN | A | 297 | −11.221 | 74.739 | 9.765 | 1.00 | 62.29 | O |
| ATOM | 2156 | NE2 | GLN | A | 297 | −11.992 | 74.570 | 11.884 | 1.00 | 60.16 | N |
| ATOM | 2157 | C | GLN | A | 297 | −11.175 | 70.550 | 12.181 | 1.00 | 51.71 | C |
| ATOM | 2158 | O | GLN | A | 297 | −11.536 | 71.140 | 13.201 | 1.00 | 52.21 | O |
| ATOM | 2159 | N | GLU | A | 298 | −11.292 | 69.234 | 12.034 | 1.00 | 51.63 | N |
| ATOM | 2160 | CA | GLU | A | 298 | −11.819 | 68.391 | 13.108 | 1.00 | 51.67 | C |
| ATOM | 2161 | CB | GLU | A | 298 | −11.714 | 66.922 | 12.736 | 1.00 | 52.61 | C |
| ATOM | 2162 | CG | GLU | A | 298 | −12.716 | 66.406 | 11.732 | 1.00 | 56.45 | C |
| ATOM | 2163 | CD | GLU | A | 298 | −12.568 | 64.908 | 11.552 | 1.00 | 60.37 | C |
| ATOM | 2164 | OE1 | GLU | A | 298 | −11.606 | 64.480 | 10.874 | 1.00 | 61.21 | O |
| ATOM | 2165 | OE2 | GLU | A | 298 | −13.403 | 64.160 | 12.112 | 1.00 | 63.66 | O |
| ATOM | 2166 | C | GLU | A | 298 | −10.991 | 68.586 | 14.372 | 1.00 | 50.78 | C |
| ATOM | 2167 | O | GLU | A | 298 | −11.523 | 68.666 | 15.490 | 1.00 | 50.03 | O |
| ATOM | 2168 | N | THR | A | 299 | −9.676 | 68.624 | 14.186 | 1.00 | 49.28 | N |
| ATOM | 2169 | CA | THR | A | 299 | −8.756 | 68.812 | 15.291 | 1.00 | 48.32 | C |
| ATOM | 2170 | CB | THR | A | 299 | −7.310 | 68.855 | 14.781 | 1.00 | 48.02 | C |
| ATOM | 2171 | OG1 | THR | A | 299 | −7.007 | 67.636 | 14.096 | 1.00 | 45.02 | O |
| ATOM | 2172 | CG2 | THR | A | 299 | −6.324 | 68.910 | 15.951 | 1.00 | 47.18 | C |
| ATOM | 2173 | C | THR | A | 299 | −9.072 | 70.095 | 16.040 | 1.00 | 48.76 | C |
| ATOM | 2174 | O | THR | A | 299 | −9.135 | 70.101 | 17.268 | 1.00 | 47.62 | O |
| ATOM | 2175 | N | ALA | A | 300 | −9.252 | 71.181 | 15.293 | 1.00 | 49.46 | N |
| ATOM | 2176 | CA | ALA | A | 300 | −9.540 | 72.468 | 15.887 | 1.00 | 50.94 | C |
| ATOM | 2177 | CB | ALA | A | 300 | −9.541 | 73.556 | 14.820 | 1.00 | 50.83 | C |
| ATOM | 2178 | C | ALA | A | 300 | −10.875 | 72.438 | 16.664 | 1.00 | 51.99 | C |
| ATOM | 2179 | O | ALA | A | 300 | −10.961 | 72.940 | 17.793 | 1.00 | 51.96 | O |
| ATOM | 2180 | N | GLU | A | 301 | −11.896 | 71.832 | 16.064 | 1.00 | 53.05 | N |
| ATOM | 2181 | CA | GLU | A | 301 | −13.218 | 71.757 | 16.689 | 1.00 | 54.49 | C |
| ATOM | 2182 | CB | GLU | A | 301 | −14.220 | 71.094 | 15.754 | 1.00 | 55.03 | C |
| ATOM | 2183 | CG | GLU | A | 301 | −14.926 | 72.073 | 14.831 | 1.00 | 58.47 | C |
| ATOM | 2184 | CD | GLU | A | 301 | −15.129 | 71.518 | 13.429 | 1.00 | 61.94 | C |
| ATOM | 2185 | OE1 | GLU | A | 301 | −15.418 | 70.303 | 13.287 | 1.00 | 62.49 | O |
| ATOM | 2186 | OE2 | GLU | A | 301 | −15.006 | 72.306 | 12.459 | 1.00 | 64.87 | O |
| ATOM | 2187 | C | GLU | A | 301 | −13.177 | 71.018 | 18.026 | 1.00 | 54.40 | C |
| ATOM | 2188 | O | GLU | A | 301 | −13.652 | 71.536 | 19.048 | 1.00 | 54.62 | O |
| ATOM | 2189 | N | ILE | A | 302 | −12.581 | 69.826 | 18.011 | 1.00 | 54.14 | N |
| ATOM | 2190 | CA | ILE | A | 302 | −12.487 | 68.970 | 19.196 | 1.00 | 53.52 | C |
| ATOM | 2191 | CB | ILE | A | 302 | −12.124 | 67.529 | 18.791 | 1.00 | 53.66 | C |
| ATOM | 2192 | CG1 | ILE | A | 302 | −13.150 | 66.981 | 17.795 | 1.00 | 53.26 | C |
| ATOM | 2193 | CD1 | ILE | A | 302 | −12.813 | 65.609 | 17.254 | 1.00 | 52.59 | C |
| ATOM | 2194 | CG2 | ILE | A | 302 | −12.047 | 66.624 | 20.026 | 1.00 | 53.65 | C |
| ATOM | 2195 | C | ILE | A | 302 | −11.496 | 69.462 | 20.246 | 1.00 | 53.50 | C |
| ATOM | 2196 | O | ILE | A | 302 | −11.800 | 69.422 | 21.440 | 1.00 | 53.33 | O |
| ATOM | 2197 | N | HIS | A | 303 | −10.322 | 69.937 | 19.822 | 1.00 | 53.16 | N |
| ATOM | 2198 | CA | HIS | A | 303 | −9.258 | 70.220 | 20.793 | 1.00 | 53.02 | C |
| ATOM | 2199 | CB | HIS | A | 303 | −8.018 | 69.390 | 20.459 | 1.00 | 51.63 | C |
| ATOM | 2200 | CG | HIS | A | 303 | −8.212 | 67.926 | 20.680 | 1.00 | 47.34 | C |
| ATOM | 2201 | ND1 | HIS | A | 303 | −8.396 | 67.043 | 19.640 | 1.00 | 45.24 | N |
| ATOM | 2202 | CE1 | HIS | A | 303 | −8.540 | 65.822 | 20.119 | 1.00 | 42.60 | C |
| ATOM | 2203 | NE2 | HIS | A | 303 | −8.456 | 65.883 | 21.437 | 1.00 | 42.67 | N |
| ATOM | 2204 | CD2 | HIS | A | 303 | −8.251 | 67.188 | 21.815 | 1.00 | 43.90 | C |
| ATOM | 2205 | C | HIS | A | 303 | −8.861 | 71.671 | 20.960 | 1.00 | 54.55 | C |
| ATOM | 2206 | O | HIS | A | 303 | −8.265 | 72.036 | 21.979 | 1.00 | 54.31 | O |
| ATOM | 2207 | N | LEU | A | 304 | −9.168 | 72.500 | 19.966 | 1.00 | 56.55 | N |
| ATOM | 2208 | CA | LEU | A | 304 | −8.696 | 73.876 | 19.999 | 1.00 | 59.36 | C |
| ATOM | 2209 | CB | LEU | A | 304 | −7.967 | 74.211 | 18.701 | 1.00 | 58.46 | C |
| ATOM | 2210 | CG | LEU | A | 304 | −6.479 | 73.870 | 18.549 | 1.00 | 58.16 | C |
| ATOM | 2211 | CD1 | LEU | A | 304 | −6.026 | 72.669 | 19.390 | 1.00 | 55.35 | C |
| ATOM | 2212 | CD2 | LEU | A | 304 | −6.160 | 73.653 | 17.061 | 1.00 | 56.12 | C |
| ATOM | 2213 | C | LEU | A | 304 | −9.832 | 74.873 | 20.273 | 1.00 | 62.12 | C |
| ATOM | 2214 | O | LEU | A | 304 | −9.586 | 76.067 | 20.431 | 1.00 | 62.18 | O |
| ATOM | 2215 | N | HIS | A | 305 | −11.061 | 74.361 | 20.340 | 1.00 | 65.89 | N |
| ATOM | 2216 | CA | HIS | A | 305 | −12.278 | 75.150 | 20.571 | 1.00 | 69.84 | C |
| ATOM | 2217 | CB | HIS | A | 305 | −12.201 | 75.963 | 21.884 | 1.00 | 70.73 | C |
| ATOM | 2218 | CG | HIS | A | 305 | −11.780 | 75.138 | 23.069 | 1.00 | 74.80 | C |
| ATOM | 2219 | ND1 | HIS | A | 305 | −12.611 | 74.206 | 23.664 | 1.00 | 77.77 | N |
| ATOM | 2220 | CE1 | HIS | A | 305 | −11.976 | 73.629 | 24.674 | 1.00 | 78.93 | C |
| ATOM | 2221 | NE2 | HIS | A | 305 | −10.760 | 74.149 | 24.753 | 1.00 | 79.02 | N |
| ATOM | 2222 | CD2 | HIS | A | 305 | −10.611 | 75.093 | 23.760 | 1.00 | 77.72 | C |
| ATOM | 2223 | C | HIS | A | 305 | −12.591 | 76.047 | 19.382 | 1.00 | 71.35 | C |
| ATOM | 2224 | O | HIS | A | 305 | −12.458 | 77.272 | 19.463 | 1.00 | 72.07 | O |
| ATOM | 2225 | N | SER | A | 306 | −12.998 | 75.426 | 18.275 | 1.00 | 73.04 | N |
| ATOM | 2226 | CA | SER | A | 306 | −13.372 | 76.161 | 17.066 | 1.00 | 74.54 | C |
| ATOM | 2227 | CB | SER | A | 306 | −12.563 | 75.685 | 15.850 | 1.00 | 74.28 | C |
| ATOM | 2228 | OG | SER | A | 306 | −11.270 | 76.309 | 15.843 | 1.00 | 74.68 | O |
| ATOM | 2229 | C | SER | A | 306 | −14.878 | 76.061 | 16.804 | 1.00 | 75.41 | C |
| ATOM | 2230 | O | SER | A | 306 | −15.588 | 77.080 | 16.858 | 1.00 | 76.03 | O |
| ATOM | 2231 | OXT | SER | A | 306 | −15.397 | 74.966 | 16.542 | 1.00 | 75.98 | O |
| ATOM | 2232 | N3 | IMD | I | 1 | 8.128 | 71.298 | 26.439 | 1.00 | 62.13 | N |
| ATOM | 2233 | C4 | IMD | I | 1 | 8.441 | 71.428 | 27.755 | 1.00 | 62.64 | C |
| ATOM | 2234 | C5 | IMD | I | 1 | 7.731 | 72.513 | 28.267 | 1.00 | 61.10 | C |
| ATOM | 2235 | C2 | IMD | I | 1 | 7.245 | 72.276 | 26.125 | 1.00 | 61.77 | C |
| ATOM | 2236 | N1 | IMD | I | 1 | 7.001 | 73.016 | 27.242 | 1.00 | 61.00 | N |
| ATOM | 2237 | O | HOH | W | 1 | −0.732 | 54.528 | 9.728 | 1.00 | 45.36 | O |
| ATOM | 2238 | O | HOH | W | 2 | 19.630 | 58.716 | 6.576 | 1.00 | 43.01 | O |
| ATOM | 2239 | O | HOH | W | 3 | 0.310 | 61.264 | 2.849 | 1.00 | 32.73 | O |
| ATOM | 2240 | O | HOH | W | 4 | 18.440 | 64.206 | 21.527 | 1.00 | 32.96 | O |
| ATOM | 2241 | O | HOH | W | 5 | 12.988 | 80.668 | 8.424 | 1.00 | 39.01 | O |
| ATOM | 2242 | O | HOH | W | 6 | −1.368 | 51.617 | 30.489 | 1.00 | 40.35 | O |
| ATOM | 2243 | O | HOH | W | 7 | 16.488 | 75.633 | 10.896 | 1.00 | 39.22 | O |
| ATOM | 2244 | O | HOH | W | 8 | 22.715 | 62.695 | 4.286 | 1.00 | 41.65 | O |
| ATOM | 2245 | O | HOH | W | 9 | 15.546 | 67.975 | 9.969 | 1.00 | 34.80 | O |
| ATOM | 2246 | O | HOH | W | 10 | 9.873 | 57.733 | 3.200 | 1.00 | 34.66 | O |
| ATOM | 2247 | O | HOH | W | 11 | 22.041 | 77.197 | 8.223 | 1.00 | 59.58 | O |
| ATOM | 2248 | O | HOH | W | 12 | 13.921 | 68.295 | 7.801 | 1.00 | 43.48 | O |
| ATOM | 2249 | O | HOH | W | 13 | −2.001 | 49.454 | 29.335 | 1.00 | 40.96 | O |
| ATOM | 2250 | O | HOH | W | 14 | 22.261 | 59.914 | 10.882 | 1.00 | 37.32 | O |
| ATOM | 2251 | O | HOH | W | 15 | 19.419 | 50.734 | 16.966 | 1.00 | 40.82 | O |
| ATOM | 2252 | O | HOH | W | 16 | 15.338 | 57.159 | 9.022 | 1.00 | 39.03 | O |
| ATOM | 2253 | O | HOH | W | 17 | 17.961 | 66.549 | 9.882 | 1.00 | 39.50 | O |
| ATOM | 2254 | O | HOH | W | 18 | 4.818 | 76.341 | 0.545 | 1.00 | 44.94 | O |
| ATOM | 2255 | O | HOH | W | 19 | 8.855 | 79.196 | 7.518 | 1.00 | 39.17 | O |
| ATOM | 2256 | O | HOH | W | 20 | 17.072 | 54.130 | 21.844 | 1.00 | 43.20 | O |
| ATOM | 2257 | O | HOH | W | 21 | 1.325 | 69.587 | 7.110 | 1.00 | 36.36 | O |
| ATOM | 2258 | O | HOH | W | 22 | 8.150 | 61.656 | 1.220 | 1.00 | 40.26 | O |
| ATOM | 2259 | O | HOH | W | 23 | −4.435 | 66.666 | 10.979 | 1.00 | 43.16 | O |
| ATOM | 2260 | O | HOH | W | 24 | 10.513 | 80.713 | 9.117 | 1.00 | 42.28 | O |
| ATOM | 2261 | O | HOH | W | 25 | 15.497 | 65.164 | 24.557 | 1.00 | 34.79 | O |
| ATOM | 2262 | O | HOH | W | 26 | 9.900 | 52.831 | 3.589 | 1.00 | 42.40 | O |
| ATOM | 2263 | O | HOH | W | 27 | −0.200 | 71.719 | 8.387 | 1.00 | 41.34 | O |
| ATOM | 2264 | O | HOH | W | 28 | −7.398 | 59.982 | 15.551 | 1.00 | 40.20 | O |
| ATOM | 2265 | O | HOH | W | 29 | 3.492 | 81.322 | 21.013 | 1.00 | 47.98 | O |
| ATOM | 2266 | O | HOH | W | 30 | −4.714 | 67.425 | 25.026 | 1.00 | 43.45 | O |
| ATOM | 2267 | O | HOH | W | 31 | 15.251 | 68.122 | 12.673 | 1.00 | 36.68 | O |
| ATOM | 2268 | O | HOH | W | 32 | −5.709 | 62.260 | 15.119 | 1.00 | 39.90 | O |
| ATOM | 2269 | O | HOH | W | 33 | 4.553 | 83.955 | 11.446 | 1.00 | 45.99 | O |
| ATOM | 2270 | O | HOH | W | 34 | 18.791 | 57.169 | 28.057 | 1.00 | 43.68 | O |
| ATOM | 2271 | O | HOH | W | 35 | 18.231 | 65.464 | 14.872 | 1.00 | 37.87 | O |
| ATOM | 2272 | O | HOH | W | 36 | 8.971 | 53.789 | 30.860 | 1.00 | 43.70 | O |
| ATOM | 2273 | O | HOH | W | 37 | 5.180 | 50.983 | 9.900 | 1.00 | 39.96 | O |
| ATOM | 2274 | O | HOH | W | 38 | −4.081 | 60.211 | 25.479 | 1.00 | 43.04 | O |
| ATOM | 2275 | O | HOH | W | 39 | −1.650 | 50.298 | 24.953 | 1.00 | 50.05 | O |
| ATOM | 2276 | O | HOH | W | 40 | −0.323 | 79.686 | 2.181 | 1.00 | 64.08 | O |
| ATOM | 2277 | O | HOH | W | 41 | −4.014 | 58.332 | 9.232 | 1.00 | 45.77 | O |
| ATOM | 2278 | O | HOH | W | 42 | 10.273 | 50.306 | 18.899 | 1.00 | 43.91 | O |
| ATOM | 2279 | O | HOH | W | 43 | 16.890 | 54.883 | 8.955 | 1.00 | 43.73 | O |
| ATOM | 2280 | O | HOH | W | 44 | 3.730 | 65.993 | 2.097 | 1.00 | 44.04 | O |
| ATOM | 2281 | O | HOH | W | 45 | 23.972 | 70.563 | 2.275 | 1.00 | 40.95 | O |
| ATOM | 2282 | O | HOH | W | 46 | 24.633 | 58.602 | 10.052 | 1.00 | 42.68 | O |
| ATOM | 2283 | O | HOH | W | 47 | 19.828 | 61.618 | 4.358 | 1.00 | 51.38 | O |
| ATOM | 2284 | O | HOH | W | 48 | 22.517 | 90.823 | 15.952 | 1.00 | 70.96 | O |
| ATOM | 2285 | O | HOH | W | 49 | 29.354 | 60.921 | 3.167 | 1.00 | 57.58 | O |
| ATOM | 2286 | O | HOH | W | 50 | 11.468 | 82.369 | 12.289 | 1.00 | 50.02 | O |
| ATOM | 2287 | O | HOH | W | 51 | 24.772 | 62.519 | −4.121 | 1.00 | 45.22 | O |
| ATOM | 2288 | O | HOH | W | 52 | 3.211 | 68.554 | 31.582 | 1.00 | 69.57 | O |
| ATOM | 2289 | O | HOH | W | 53 | 7.936 | 50.002 | 23.124 | 1.00 | 47.40 | O |
| ATOM | 2290 | O | HOH | W | 54 | 15.587 | 71.212 | 17.046 | 1.00 | 52.35 | O |
| ATOM | 2291 | O | HOH | W | 55 | 15.884 | 79.008 | −3.580 | 1.00 | 56.72 | O |
| ATOM | 2292 | O | HOH | W | 56 | 25.279 | 56.110 | 10.230 | 1.00 | 44.21 | O |
| ATOM | 2293 | O | HOH | W | 57 | 12.514 | 58.767 | 4.837 | 1.00 | 52.23 | O |
| ATOM | 2294 | O | HOH | W | 58 | 1.688 | 78.234 | 4.543 | 1.00 | 43.74 | O |
| ATOM | 2295 | O | HOH | W | 59 | 9.018 | 82.803 | 11.168 | 1.00 | 50.76 | O |
| ATOM | 2296 | O | HOH | W | 60 | −0.217 | 85.742 | 6.096 | 1.00 | 53.93 | O |
| ATOM | 2297 | O | HOH | W | 61 | −2.930 | 82.309 | 21.772 | 1.00 | 58.06 | O |
| ATOM | 2298 | O | HOH | W | 62 | 5.504 | 51.225 | 5.130 | 1.00 | 48.90 | O |
| ATOM | 2299 | O | HOH | W | 63 | 20.076 | 54.469 | 7.350 | 1.00 | 61.18 | O |
| ATOM | 2300 | O | HOH | W | 64 | 5.722 | 68.809 | −1.934 | 1.00 | 59.42 | O |
| ATOM | 2301 | O | HOH | W | 65 | 27.882 | 66.292 | −1.512 | 1.00 | 65.79 | O |
| ATOM | 2302 | O | HOH | W | 66 | 19.676 | 72.153 | 23.229 | 1.00 | 61.17 | O |
| ATOM | 2303 | O | HOH | W | 67 | −5.501 | 71.414 | 5.301 | 1.00 | 61.16 | O |
| ATOM | 2304 | O | HOH | W | 68 | 15.016 | 58.056 | 6.473 | 1.00 | 49.90 | O |
| ATOM | 2305 | O | HOH | W | 69 | −2.012 | 55.730 | 6.130 | 1.00 | 56.99 | O |
| ATOM | 2306 | O | HOH | W | 70 | −9.447 | 70.188 | 7.682 | 1.00 | 57.22 | O |
| ATOM | 2307 | O | HOH | W | 71 | 2.484 | 55.120 | −0.038 | 1.00 | 49.25 | O |
| ATOM | 2308 | O | HOH | W | 72 | −7.908 | 59.237 | 8.479 | 1.00 | 65.73 | O |
| ATOM | 2309 | O | HOH | W | 73 | 22.353 | 73.255 | 11.764 | 1.00 | 60.74 | O |
| ATOM | 2310 | O | HOH | W | 74 | 19.477 | 67.324 | 11.857 | 1.00 | 50.67 | O |
| ATOM | 2311 | O | HOH | W | 75 | 14.506 | 47.970 | 13.280 | 1.00 | 62.36 | O |
| ATOM | 2312 | O | HOH | W | 76 | 16.862 | 47.807 | 8.768 | 1.00 | 52.55 | O |
| ATOM | 2313 | O | HOH | W | 77 | 13.313 | 53.083 | 32.098 | 1.00 | 54.43 | O |
| ATOM | 2314 | O | HOH | W | 78 | 17.503 | 50.798 | 19.041 | 1.00 | 55.72 | O |
| ATOM | 2315 | O | HOH | W | 79 | −12.736 | 62.094 | 18.189 | 1.00 | 53.56 | O |
| ATOM | 2316 | O | HOH | W | 80 | 33.908 | 62.979 | 10.712 | 1.00 | 63.79 | O |
| ATOM | 2317 | O | HOH | W | 81 | −6.870 | 60.605 | 22.628 | 1.00 | 49.67 | O |
| ATOM | 2318 | O | HOH | W | 82 | 9.987 | 47.582 | 14.046 | 1.00 | 66.52 | O |
| ATOM | 2319 | O | HOH | W | 83 | 23.183 | 73.287 | 2.510 | 1.00 | 52.15 | O |
| ATOM | 2320 | O | HOH | W | 84 | 27.578 | 55.770 | 9.060 | 1.00 | 63.00 | O |
| ATOM | 2321 | O | HOH | W | 85 | 5.576 | 82.970 | −1.748 | 1.00 | 62.58 | O |
| ATOM | 2322 | O | HOH | W | 86 | −0.509 | 84.330 | 3.857 | 1.00 | 59.32 | O |
| ATOM | 2323 | O | HOH | W | 87 | 13.665 | 91.473 | −3.552 | 1.00 | 61.08 | O |
| ATOM | 2324 | O | HOH | W | 88 | −2.861 | 75.397 | −2.038 | 1.00 | 63.22 | O |
| ATOM | 2325 | O | HOH | W | 89 | 10.204 | 73.979 | 29.790 | 1.00 | 66.32 | O |
| ATOM | 2326 | O | HOH | W | 90 | 20.070 | 78.983 | 6.971 | 1.00 | 67.67 | O |
| ATOM | 2327 | O | HOH | W | 91 | 17.169 | 77.347 | 21.910 | 1.00 | 65.17 | O |
| ATOM | 2328 | O | HOH | W | 92 | −2.870 | 53.045 | 18.163 | 1.00 | 59.47 | O |
| ATOM | 2329 | O | HOH | W | 93 | 11.627 | 71.628 | 23.440 | 1.00 | 63.19 | O |
| ATOM | 2330 | O | HOH | W | 94 | 8.310 | 74.960 | −2.678 | 1.00 | 55.47 | O |
| ATOM | 2331 | O | HOH | W | 95 | −12.002 | 78.851 | 4.304 | 1.00 | 58.94 | O |
| ATOM | 2332 | O | HOH | W | 96 | 5.566 | 49.157 | 22.796 | 1.00 | 51.78 | O |
| ATOM | 2333 | O | HOH | W | 97 | 31.358 | 61.478 | 0.597 | 1.00 | 66.61 | O |
| ATOM | 2334 | O | HOH | W | 98 | 24.035 | 64.093 | −2.091 | 1.00 | 47.25 | O |
| ATOM | 2335 | O | HOH | W | 99 | 11.294 | 69.111 | 34.295 | 1.00 | 70.13 | O |
| ATOM | 2336 | O | HOH | W | 100 | 18.999 | 64.123 | −2.168 | 1.00 | 62.14 | O |
| ATOM | 2337 | O | HOH | W | 101 | −9.739 | 61.493 | 7.698 | 1.00 | 82.68 | O |
| ATOM | 2338 | O | HOH | W | 102 | 22.435 | 52.025 | 25.439 | 1.00 | 54.62 | O |
| ATOM | 2339 | O | HOH | W | 103 | 5.045 | 49.276 | 12.114 | 1.00 | 55.83 | O |
| ATOM | 2340 | O | HOH | W | 104 | −3.965 | 50.524 | 12.224 | 1.00 | 62.13 | O |
| ATOM | 2341 | O | HOH | W | 105 | 13.472 | 75.945 | 26.250 | 1.00 | 61.94 | O |
| ATOM | 2342 | O | HOH | W | 106 | 15.560 | 72.156 | 26.297 | 1.00 | 58.39 | O |
| ATOM | 2343 | O | HOH | W | 107 | −0.195 | 96.034 | 19.635 | 1.00 | 69.60 | O |
| ATOM | 2344 | O | HOH | W | 108 | 1.243 | 88.090 | −4.031 | 1.00 | 62.22 | O |
| ATOM | 2345 | O | HOH | W | 109 | 19.973 | 83.759 | 20.585 | 1.00 | 71.41 | O |
| ATOM | 2346 | O | HOH | W | 110 | −8.152 | 73.486 | 8.288 | 1.00 | 53.47 | O |
| ATOM | 2347 | O | HOH | W | 111 | 23.420 | 81.722 | 9.233 | 1.00 | 71.36 | O |
| ATOM | 2348 | O | HOH | W | 112 | 1.596 | 82.691 | −0.096 | 1.00 | 71.76 | O |
| ATOM | 2349 | O | HOH | W | 113 | 5.657 | 56.059 | −1.336 | 1.00 | 64.94 | O |
| ATOM | 2350 | O | HOH | W | 114 | 13.967 | 51.575 | 8.374 | 1.00 | 51.56 | O |
| ATOM | 2351 | O | HOH | W | 115 | 12.416 | 78.389 | 25.200 | 1.00 | 66.19 | O |
| ATOM | 2352 | O | HOH | W | 116 | 17.235 | 83.392 | 11.447 | 1.00 | 52.25 | O |
| ATOM | 2353 | O | HOH | W | 117 | 14.767 | 52.852 | 21.314 | 1.00 | 47.79 | O |
| ATOM | 2354 | O | HOH | W | 118 | 19.075 | 60.231 | −4.028 | 1.00 | 64.68 | O |
| ATOM | 2355 | O | HOH | W | 119 | 25.476 | 66.800 | 28.823 | 1.00 | 55.96 | O |
| ATOM | 2356 | O | HOH | W | 120 | 4.473 | 70.021 | 30.020 | 1.00 | 56.80 | O |
| ATOM | 2357 | O | HOH | W | 121 | 8.400 | 80.051 | 18.580 | 1.00 | 47.86 | O |
| ATOM | 2358 | O | HOH | W | 122 | −0.274 | 81.374 | 6.467 | 1.00 | 70.59 | O |
| ATOM | 2359 | O | HOH | W | 123 | 8.016 | 51.083 | 3.826 | 1.00 | 50.11 | O |
| ATOM | 2360 | O | HOH | W | 124 | −5.762 | 55.323 | 8.603 | 1.00 | 59.77 | O |
| ATOM | 2361 | O | HOH | W | 125 | 24.801 | 94.210 | −1.115 | 1.00 | 67.33 | O |
| ATOM | 2362 | O | HOH | W | 126 | 9.710 | 48.669 | 26.328 | 1.00 | 63.06 | O |
| ATOM | 2363 | O | HOH | W | 127 | 8.684 | 99.063 | 14.167 | 1.00 | 63.47 | O |
| ATOM | 2364 | O | HOH | W | 128 | 19.451 | 83.648 | 8.511 | 1.00 | 51.41 | O |
| ATOM | 2365 | O | HOH | W | 129 | −10.889 | 61.955 | 10.215 | 1.00 | 55.28 | O |
| ATOM | 2366 | O | HOH | W | 130 | −4.253 | 61.866 | 27.652 | 1.00 | 61.67 | O |
| ATOM | 2367 | O | HOH | W | 131 | 27.030 | 90.340 | 3.848 | 1.00 | 80.85 | O |
| ATOM | 2368 | O | HOH | W | 132 | 10.977 | 87.131 | 22.623 | 1.00 | 65.06 | O |
| ATOM | 2369 | O | HOH | W | 133 | 14.634 | 65.394 | −2.521 | 1.00 | 56.18 | O |
| ATOM | 2370 | O | HOH | W | 134 | −3.405 | 52.808 | 20.692 | 1.00 | 57.93 | O |
| ATOM | 2371 | O | HOH | W | 135 | −5.420 | 55.451 | 15.525 | 1.00 | 51.90 | O |
| ATOM | 2372 | O | HOH | W | 136 | 8.056 | 79.671 | 22.675 | 1.00 | 59.54 | O |
| ATOM | 2373 | O | HOH | W | 137 | 28.392 | 57.786 | 4.755 | 1.00 | 78.57 | O |
| ATOM | 2374 | O | HOH | W | 138 | 18.312 | 99.689 | 9.767 | 1.00 | 61.28 | O |
| ATOM | 2375 | O | HOH | W | 139 | 33.446 | 63.253 | 17.723 | 1.00 | 59.53 | O |
| ATOM | 2376 | O | HOH | W | 140 | 24.283 | 56.206 | 17.474 | 1.00 | 54.00 | O |
| ATOM | 2377 | O | HOH | W | 141 | 16.808 | 50.392 | 32.500 | 1.00 | 57.59 | O |
| ATOM | 2378 | O | HOH | W | 142 | 15.746 | 83.812 | −7.461 | 1.00 | 64.85 | O |
| ATOM | 2379 | O | HOH | W | 143 | −7.082 | 94.424 | −2.169 | 1.00 | 67.76 | O |
| ATOM | 2380 | O | HOH | W | 144 | 13.631 | 49.312 | 10.749 | 1.00 | 54.19 | O |
| ATOM | 2381 | O | HOH | W | 145 | 30.247 | 61.193 | 23.440 | 1.00 | 71.27 | O |
| ATOM | 2382 | O | HOH | W | 146 | 13.010 | 80.075 | −6.528 | 1.00 | 68.99 | O |

[image] [image] [image] [image] [image] [image] [image] [image] [image] [image] [image] TABLE 4

| HEADER | | ---- XX-XXX-XX xxxx | | | |
|---|---|---|---|---|---|
| COMPND | | --- | | | |
| REMARK | 3 | | | | |
| REMARK | 3 | REFINEMENT. | | | |
| REMARK | 3 | PROGRAM: REFMAC 5.1.21 | | | |
| REMARK | 3 | AUTHORS: MURSHUDOV, VAGIN, DODSON | | | |
| REMARK | 3 | | | | |
| REMARK | 3 | REFINEMENT TARGET: MAXIMUM LIKELIHOOD | | | |
| REMARK | 3 | | | | |
| REMARK | 3 | DATA USED IN REFINEMENT. | | | |
| REMARK | 3 | RESOLUTION RANGE HIGH (ANGSTROMS): 2.03 | | | |
| REMARK | 3 | RESOLUTION RANGE LOW (ANGSTROMS): 81.65 | | | |
| REMARK | 3 | DATA CUTOFF (SIGMA(F)): NONE | | | |
| REMARK | 3 | COMPLETENESS FOR RANGE (%): 99.81 | | | |
| REMARK | 3 | NUMBER OF REFLECTIONS: 25766 | | | |
| REMARK | 3 | | | | |
| REMARK | 3 | FIT TO DATA USED IN REFINEMENT. | | | |
| REMARK | 3 | CROSS-VALIDATION METHOD: THROUGHOUT | | | |
| REMARK | 3 | FREE R VALUE TEST SET SELECTION: RANDOM | | | |
| REMARK | 3 | R VALUE (WORKING + TEST SET): 0.19077 | | | |
| REMARK | 3 | R VALUE (WORKING SET): 0.18920 | | | |
| REMARK | 3 | FREE R VALUE: 0.22121 | | | |
| REMARK | 3 | FREE R VALUE TEST SET SIZE (%): 5.0 | | | |
| REMARK | 3 | FREE R VALUE TEST SET COUNT: 1368 | | | |
| REMARK | 3 | | | | |
| REMARK | 3 | FIT IN THE HIGHEST RESOLUTION BIN. | | | |
| REMARK | 3 | TOTAL NUMBER OF BINS USED: 20 | | | |
| REMARK | 3 | BIN RESOLUTION RANGE HIGH: 2.030 | | | |
| REMARK | 3 | BIN RESOLUTION RANGE LOW: 2.083 | | | |
| REMARK | 3 | REFLECTION IN BIN (WORKING SET): 1894 | | | |
| REMARK | 3 | BIN R VALUE (WORKING SET): 0.289 | | | |
| REMARK | 3 | BIN FREE R VALUE SET COUNT: 113 | | | |
| REMARK | 3 | BIN FREE R VALUE: 0.297 | | | |
| REMARK | 3 | | | | |
| REMARK | 3 | NUMBER OF NON-HYDROGEN ATOMS USED IN REFINEMENT. | | | |
| REMARK | 3 | ALL ATOMS: 2400 | | | |
| REMARK | 3 | | | | |
| REMARK | 3 | B VALUES. | | | |
| REMARK | 3 | FROM WILSON PLOT (A**2): NULL | | | |
| REMARK | 3 | MEAN B VALUE (OVERALL, A**2): 27.297 | | | |
| REMARK | 3 | OVERALL ANISOTROPIC B VALUE. | | | |
| REMARK | 3 | B11 (A**2): 0.50 | | | |
| REMARK | 3 | B22 (A**2): 0.50 | | | |
| REMARK | 3 | B33 (A**2): −0.74 | | | |
| REMARK | 3 | B12 (A**2): 0.25 | | | |
| REMARK | 3 | B13 (A**2): 0.00 | | | |
| REMARK | 3 | B23 (A**2): 0.00 | | | |
| REMARK | 3 | | | | |
| REMARK | 3 | ESTIMATED OVERALL COORDINATE ERROR. | | | |
| REMARK | 3 | ESU BASED ON R VALUE (A): 0.151 | | | |
| REMARK | 3 | ESU BASED ON FREE R VALUE (A): 0.140 | | | |
| REMARK | 3 | ESU BASED ON MAXIMUM LIKELIHOOD (A): 0.105 | | | |
| REMARK | 3 | ESU FOR B VALUES BASED ON MAXIMUM LIKELIHOOD (A**2): 3.960 | | | |
| REMARK | 3 | | | | |
| REMARK | 3 | CORRELATION COEFFICIENTS. | | | |
| REMARK | 3 | CORRELATION COEFFICIENT FO-FC: 0.959 | | | |
| REMARK | 3 | CORRELATION COEFFICIENT FO-FC FREE: 0.946 | | | |
| REMARK | 3 | | | | |
| REMARK | 3 | RMS DEVIATIONS FROM IDEAL VALUES | COUNT | RMS | WEIGHT |
| REMARK | 3 | BOND LENGTHS REFINED ATOMS (A): | 2334; | 0.011; | 0.021 |
| REMARK | 3 | BOND ANGLES REFINED ATOMS (DEGREES): | 3174; | 1.105; | 1.959 |
| REMARK | 3 | TORSION ANGLES, PERIOD 1 (DEGREES): | 273; | 5.228; | 5.000 |
| REMARK | 3 | CHIRAL-CENTER RESTRAINTS (A**3): | 336; | 0.080; | 0.200 |
| REMARK | 3 | GENERAL PLANES REFINED ATOMS (A): | 1800; | 0.004; | 0.020 |
| REMARK | 3 | NON-BONDED CONTACTS REFINED ATOMS (A): | 1070; | 0.202; | 0.200 |
| REMARK | 3 | H-BOND (X . . . Y) REFINED ATOMS (A): | 150; | 0.145; | 0.200 |
| REMARK | 3 | SYMMETRY VDW REFINED ATOMS (A): | 46; | 0.199; | 0.200 |
| REMARK | 3 | SYMMETRY H-BOND REFINED ATOMS (A): | 10; | 0.267; | 0.200 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2): | 1365; | 0.799; | 1.500 |
| REMARK | 3 | MAIN-CHAIN ANGLE REFINED ATOMS (A**2): | 2214; | 1.519; | 2.000 |
| REMARK | 3 | SIDE-CHAIN BOND REFINED ATOMS (A**2): | 969; | 2.024; | 3.000 |
| REMARK | 3 | SIDE-CHAIN ANGLE REFINED ATOMS (A**2): | 960; | 3.247; | 4.500 |

| REMARK | 3 | | | | | | |
|---|---|---|---|---|---|---|---|
| REMARK | 3 | NCS RESTRAINTS STATISTICS | | | | | |
| REMARK | 3 | NUMBER OF NCS GROUPS: NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS: 2 | | | | | |
| REMARK | 3 | | | | | | |
| REMARK | 3 | TLS GROUP: 1 | | | | | |
| REMARK | 3 | NUMBER OF COMPONENTS GROUP: 1 | | | | | |
| REMARK | 3 | COMPONENTS C SSSEQI TO C SSSEQI | | | | | |
| REMARK | 3 | RESIDUE RANGE: A 33 A 306 | | | | | |
| REMARK | 3 | ORIGIN FOR THE GROUP (A): 65.5800 27.1270 −0.6960 | | | | | |
| REMARK | 3 | T TENSOR | | | | | |
| REMARK | 3 | T11: | 0.1410 | T22: | 0.1266 | | |
| REMARK | 3 | T33: | 0.0824 | T12: | −0.0364 | | |
| REMARK | 3 | T13: | −0.0112 | T23: | −0.0301 | | |
| REMARK | 3 | L TENSOR | | | | | |
| REMARK | 3 | L11: | 1.3945 | L22: | 0.7253 | | |
| REMARK | 3 | L33: | 0.8680 | L12: | 0.1248 | | |
| REMARK | 3 | L13: | −0.3386 | L23: | 0.0070 | | |
| REMARK | 3 | S TENSOR | | | | | |
| REMARK | 3 | S11: | −0.0668 | S12: | 0.0858 | S13: | 0.0787 |
| REMARK | 3 | S21: | −0.0201 | S22: | 0.1089 | S23: | 0.0287 |
| REMARK | 3 | S31: | 0.0298 | S32: | 0.0689 | S33: | −0.0421 |
| REMARK | 3 | | | | | | |
| REMARK | 3 | TLS GROUP: 2 | | | | | |
| REMARK | 3 | NUMBER OF COMPONENTS GROUP: 1 | | | | | |
| REMARK | 3 | COMPONENTS C SSSEQI TO C SSSEQI | | | | | |
| REMARK | 3 | RESIDUE RANGE: L 1 L 1 | | | | | |
| REMARK | 3 | ORIGIN FOR THE GROUP (A): 73.8810 32.6080 1.3720 | | | | | |
| REMARK | 3 | T TENSOR | | | | | |
| REMARK | 3 | T11: | 0.1174 | T22: | 0.1943 | | |
| REMARK | 3 | T33: | 0.1391 | T12: | −0.1003 | | |
| REMARK | 3 | T13: | −0.0486 | T23: | −0.0722 | | |
| REMARK | 3 | L TENSOR | | | | | |
| REMARK | 3 | L11: | 14.6629 | L22: | 15.7148 | | |
| REMARK | 3 | L33: | 8.9109 | L12: | −11.1563 | | |
| REMARK | 3 | L13: | −1.8290 | L23: | −15.1157 | | |
| REMARK | 3 | S TENSOR | | | | | |
| REMARK | 3 | S11: | 0.2483 | S12: | 0.1966 | S13: | 0.0403 |
| REMARK | 3 | S21: | 0.0302 | S22: | 0.1725 | S23: | 0.8712 |
| REMARK | 3 | S31: | −0.4688 | S32: | 0.8689 | S33: | −0.4208 |

| | | |
|---|---|---|
| REMARK | 3 | |
| REMARK | 3 | |
| REMARK | 3 | BULK SOLVENT MODELLING. |
| REMARK | 3 | METHOD USED: BABINET MODEL WITH MASK |
| REMARK | 3 | PARAMETERS FOR MASK CALCULATION |
| REMARK | 3 | VDW PROBE RADIUS: 1.40 |
| REMARK | 3 | ION PROBE RADIUS: 0.80 |
| REMARK | 3 | SHRINKAGE RADIUS: 0.80 |
| REMARK | 3 | |
| REMARK | 3 | OTHER REFINEMENT REMARKS: NULL |
| REMARK | 3 | |
| CISPEP | | 1 GLU A 124 PRO A 125 0.00 |
| CRYST1 | | 95.566 95.566 80.862 90.00 90.00 120.00 P 65 |
| SCALE1 | | 0.010464 0.006041 0.000000 0.00000 |
| SCALE2 | | 0.000000 0.012083 0.000000 0.00000 |
| SCALE3 | | 0.000000 0.000000 0.012367 0.00000 |

| | | | | | | | | | | | |
|---|---|---|---|---|---|---|---|---|---|---|---|
| ATOM | 1 | N | PRO | A | 33 | 89.149 | 40.408 | −18.445 | 1.00 | 67.30 | N |
| ATOM | 2 | CA | PRO | A | 33 | 88.476 | 41.476 | −17.647 | 1.00 | 67.14 | C |
| ATOM | 3 | CB | PRO | A | 33 | 86.997 | 41.088 | −17.742 | 1.00 | 67.23 | C |
| ATOM | 4 | CG | PRO | A | 33 | 86.877 | 40.393 | −19.088 | 1.00 | 67.40 | C |
| ATOM | 5 | CD | PRO | A | 33 | 88.243 | 39.825 | −19.451 | 1.00 | 67.35 | C |
| ATOM | 6 | C | PRO | A | 33 | 88.938 | 41.544 | −16.180 | 1.00 | 66.89 | C |
| ATOM | 7 | O | PRO | A | 33 | 89.154 | 42.657 | −15.690 | 1.00 | 67.00 | O |
| ATOM | 8 | N | LEU | A | 34 | 89.091 | 40.388 | −15.519 | 1.00 | 66.32 | N |
| ATOM | 9 | CA | LEU | A | 34 | 89.499 | 40.274 | −14.100 | 1.00 | 65.68 | C |
| ATOM | 10 | CB | LEU | A | 34 | 90.888 | 40.895 | −13.835 | 1.00 | 65.85 | C |
| ATOM | 11 | CG | LEU | A | 34 | 91.302 | 41.230 | −12.390 | 1.00 | 66.30 | C |
| ATOM | 12 | CD1 | LEU | A | 34 | 91.714 | 39.982 | −11.600 | 1.00 | 66.80 | C |
| ATOM | 13 | CD2 | LEU | A | 34 | 92.418 | 42.268 | −12.376 | 1.00 | 67.23 | C |
| ATOM | 14 | C | LEU | A | 34 | 88.454 | 40.795 | −13.100 | 1.00 | 64.94 | C |
| ATOM | 15 | O | LEU | A | 34 | 87.873 | 41.869 | −13.284 | 1.00 | 64.93 | O |
| ATOM | 16 | N | GLU | A | 35 | 88.242 | 40.036 | −12.027 | 1.00 | 63.77 | N |
| ATOM | 17 | CA | GLU | A | 35 | 87.186 | 40.343 | −11.061 | 1.00 | 62.65 | C |
| ATOM | 18 | CB | GLU | A | 35 | 86.798 | 39.087 | −10.270 | 1.00 | 63.13 | C |
| ATOM | 19 | CG | GLU | A | 35 | 87.856 | 38.599 | −9.297 | 1.00 | 65.02 | C |
| ATOM | 20 | CD | GLU | A | 35 | 87.245 | 37.871 | −8.122 | 1.00 | 67.48 | C |
| ATOM | 21 | OE1 | GLU | A | 35 | 87.017 | 38.518 | −7.069 | 1.00 | 68.53 | O |
| ATOM | 22 | OE2 | GLU | A | 35 | 86.987 | 36.654 | −8.260 | 1.00 | 68.44 | O |
| ATOM | 23 | C | GLU | A | 35 | 87.444 | 41.549 | −10.130 | 1.00 | 61.12 | C |
| ATOM | 24 | O | GLU | A | 35 | 86.859 | 41.645 | −9.049 | 1.00 | 61.05 | O |
| ATOM | 25 | N | SER | A | 36 | 88.299 | 42.477 | −10.561 | 1.00 | 59.16 | N |
| ATOM | 26 | CA | SER | A | 36 | 88.360 | 43.797 | −9.923 | 1.00 | 56.76 | C |
| ATOM | 27 | CB | SER | A | 36 | 89.767 | 44.375 | −9.977 | 1.00 | 57.09 | C |
| ATOM | 28 | OG | SER | A | 36 | 90.185 | 44.711 | −8.665 | 1.00 | 57.93 | O |
| ATOM | 29 | C | SER | A | 36 | 87.321 | 44.757 | −10.537 | 1.00 | 54.68 | C |
| ATOM | 30 | O | SER | A | 36 | 87.465 | 45.987 | −10.482 | 1.00 | 54.42 | O |
| ATOM | 31 | N | GLN | A | 37 | 86.278 | 44.159 | −11.121 | 1.00 | 51.69 | N |
| ATOM | 32 | CA | GLN | A | 37 | 85.055 | 44.844 | −11.531 | 1.00 | 48.71 | C |
| ATOM | 33 | CB | GLN | A | 37 | 84.288 | 44.000 | −12.556 | 1.00 | 48.70 | C |
| ATOM | 34 | CG | GLN | A | 37 | 85.032 | 43.705 | −13.853 | 1.00 | 48.89 | C |
| ATOM | 35 | CD | GLN | A | 37 | 84.357 | 42.614 | −14.681 | 1.00 | 48.93 | C |
| ATOM | 36 | OE1 | GLN | A | 37 | 83.235 | 42.790 | −15.159 | 1.00 | 48.57 | O |
| ATOM | 37 | NE2 | GLN | A | 37 | 85.041 | 41.490 | −14.849 | 1.00 | 49.23 | N |
| ATOM | 38 | C | GLN | A | 37 | 84.159 | 45.068 | −10.309 | 1.00 | 46.45 | C |
| ATOM | 39 | O | GLN | A | 37 | 83.061 | 45.621 | −10.425 | 1.00 | 45.80 | O |
| ATOM | 40 | N | TYR | A | 38 | 84.634 | 44.634 | −9.142 | 1.00 | 43.79 | N |
| ATOM | 41 | CA | TYR | A | 38 | 83.815 | 44.599 | −7.935 | 1.00 | 41.47 | C |
| ATOM | 42 | CB | TYR | A | 38 | 83.277 | 43.178 | −7.683 | 1.00 | 40.80 | C |
| ATOM | 43 | CG | TYR | A | 38 | 82.415 | 42.680 | −8.813 | 1.00 | 37.71 | C |
| ATOM | 44 | CD1 | TYR | A | 38 | 81.078 | 43.060 | −8.913 | 1.00 | 36.03 | C |
| ATOM | 45 | CE1 | TYR | A | 38 | 80.283 | 42.622 | −9.970 | 1.00 | 34.69 | C |
| ATOM | 46 | CZ | TYR | A | 38 | 80.834 | 41.799 | −10.940 | 1.00 | 33.35 | C |
| ATOM | 47 | OH | TYR | A | 38 | 80.058 | 41.362 | −11.982 | 1.00 | 33.33 | O |
| ATOM | 48 | CE2 | TYR | A | 38 | 82.156 | 41.409 | −10.861 | 1.00 | 33.59 | C |
| ATOM | 49 | CD2 | TYR | A | 38 | 82.941 | 41.853 | −9.801 | 1.00 | 35.25 | C |
| ATOM | 50 | C | TYR | A | 38 | 84.546 | 45.110 | −6.711 | 1.00 | 40.70 | C |
| ATOM | 51 | O | TYR | A | 38 | 85.729 | 44.835 | −6.522 | 1.00 | 40.55 | O |
| ATOM | 52 | N | GLN | A | 39 | 83.820 | 45.882 | −5.907 | 1.00 | 39.47 | N |
| ATOM | 53 | CA | GLN | A | 39 | 84.267 | 46.331 | −4.602 | 1.00 | 38.56 | C |
| ATOM | 54 | CB | GLN | A | 39 | 83.781 | 47.755 | −4.350 | 1.00 | 39.00 | C |
| ATOM | 55 | CG | GLN | A | 39 | 84.391 | 48.448 | −3.148 | 1.00 | 41.12 | C |
| ATOM | 56 | CD | GLN | A | 39 | 83.988 | 49.920 | −3.066 | 1.00 | 44.96 | C |
| ATOM | 57 | OE1 | GLN | A | 39 | 84.489 | 50.753 | −3.833 | 1.00 | 45.76 | O |
| ATOM | 58 | NE2 | GLN | A | 39 | 83.081 | 50.241 | −2.139 | 1.00 | 46.18 | N |
| ATOM | 59 | C | GLN | A | 39 | 83.673 | 45.376 | −3.567 | 1.00 | 37.29 | C |
| ATOM | 60 | O | GLN | A | 39 | 82.451 | 45.224 | −3.473 | 1.00 | 36.66 | O |
| ATOM | 61 | N | VAL | A | 40 | 84.546 | 44.738 | −2.797 | 1.00 | 35.79 | N |
| ATOM | 62 | CA | VAL | A | 40 | 84.124 | 43.757 | −1.808 | 1.00 | 34.34 | C |
| ATOM | 63 | CB | VAL | A | 40 | 85.190 | 42.667 | −1.580 | 1.00 | 34.42 | C |
| ATOM | 64 | CG1 | VAL | A | 40 | 84.573 | 41.470 | −0.871 | 1.00 | 34.56 | C |
| ATOM | 65 | CG2 | VAL | A | 40 | 85.822 | 42.238 | −2.908 | 1.00 | 34.84 | C |
| ATOM | 66 | C | VAL | A | 40 | 83.794 | 44.435 | −0.487 | 1.00 | 33.17 | C |
| ATOM | 67 | O | VAL | A | 40 | 84.544 | 45.296 | −0.021 | 1.00 | 32.88 | O |
| ATOM | 68 | N | GLY | A | 41 | 82.659 | 44.047 | 0.094 | 1.00 | 31.35 | N |
| ATOM | 69 | CA | GLY | A | 41 | 82.253 | 44.508 | 1.407 | 1.00 | 29.70 | C |
| ATOM | 70 | C | GLY | A | 41 | 82.304 | 43.395 | 2.438 | 1.00 | 28.50 | C |
| ATOM | 71 | O | GLY | A | 41 | 83.121 | 42.480 | 2.315 | 1.00 | 28.62 | O |
| ATOM | 72 | N | PRO | A | 42 | 81.435 | 43.470 | 3.446 | 1.00 | 27.60 | N |
| ATOM | 73 | CA | PRO | A | 42 | 81.406 | 42.494 | 4.543 | 1.00 | 27.00 | C |
| ATOM | 74 | CB | PRO | A | 42 | 80.355 | 43.073 | 5.501 | 1.00 | 27.10 | C |
| ATOM | 75 | CG | PRO | A | 42 | 80.182 | 44.506 | 5.089 | 1.00 | 27.33 | C |
| ATOM | 76 | CD | PRO | A | 42 | 80.416 | 44.521 | 3.618 | 1.00 | 27.63 | C |
| ATOM | 77 | C | PRO | A | 42 | 80.958 | 41.091 | 4.127 | 1.00 | 26.95 | C |
| ATOM | 78 | O | PRO | A | 42 | 80.212 | 40.921 | 3.149 | 1.00 | 26.22 | O |
| ATOM | 79 | N | LEU | A | 43 | 81.421 | 40.114 | 4.905 | 1.00 | 26.40 | N |
| ATOM | 80 | CA | LEU | A | 43 | 81.016 | 38.726 | 4.827 | 1.00 | 26.26 | C |
| ATOM | 81 | CB | LEU | A | 43 | 81.928 | 37.888 | 5.737 | 1.00 | 26.16 | C |
| ATOM | 82 | CG | LEU | A | 43 | 81.741 | 36.367 | 5.836 | 1.00 | 26.71 | C |
| ATOM | 83 | CD1 | LEU | A | 43 | 81.971 | 35.666 | 4.486 | 1.00 | 25.17 | C |
| ATOM | 84 | CD2 | LEU | A | 43 | 82.656 | 35.790 | 6.911 | 1.00 | 25.63 | C |
| ATOM | 85 | C | LEU | A | 43 | 79.573 | 38.592 | 5.292 | 1.00 | 26.23 | C |
| ATOM | 86 | O | LEU | A | 43 | 79.234 | 39.010 | 6.409 | 1.00 | 25.53 | O |
| ATOM | 87 | N | LEU | A | 44 | 78.737 | 37.998 | 4.439 | 1.00 | 25.89 | N |
| ATOM | 88 | CA | LEU | A | 44 | 77.321 | 37.786 | 4.746 | 1.00 | 26.23 | C |
| ATOM | 89 | CB | LEU | A | 44 | 76.460 | 37.994 | 3.500 | 1.00 | 25.73 | C |
| ATOM | 90 | CG | LEU | A | 44 | 76.500 | 39.383 | 2.881 | 1.00 | 25.94 | C |
| ATOM | 91 | CD1 | LEU | A | 44 | 75.804 | 39.381 | 1.516 | 1.00 | 24.87 | C |
| ATOM | 92 | CD2 | LEU | A | 44 | 75.881 | 40.399 | 3.846 | 1.00 | 26.24 | C |
| ATOM | 93 | C | LEU | A | 44 | 77.027 | 36.416 | 5.345 | 1.00 | 26.74 | C |
| ATOM | 94 | O | LEU | A | 44 | 76.107 | 36.274 | 6.148 | 1.00 | 26.63 | O |
| ATOM | 95 | N | GLY | A | 45 | 77.798 | 35.409 | 4.946 | 1.00 | 27.42 | N |
| ATOM | 96 | CA | GLY | A | 45 | 77.595 | 34.056 | 5.434 | 1.00 | 28.81 | C |
| ATOM | 97 | C | GLY | A | 45 | 78.642 | 33.077 | 4.932 | 1.00 | 29.90 | C |
| ATOM | 98 | O | GLY | A | 45 | 79.209 | 33.254 | 3.854 | 1.00 | 29.36 | O |
| ATOM | 99 | N | SER | A | 46 | 78.908 | 32.061 | 5.745 | 1.00 | 31.14 | N |
| ATOM | 100 | CA | SER | A | 46 | 79.794 | 30.964 | 5.385 | 1.00 | 33.14 | C |
| ATOM | 101 | CB | SER | A | 46 | 81.242 | 31.282 | 5.786 | 1.00 | 33.24 | C |
| ATOM | 102 | OG | SER | A | 46 | 81.336 | 31.607 | 7.161 | 1.00 | 31.40 | O |
| ATOM | 103 | C | SER | A | 46 | 79.263 | 29.723 | 6.104 | 1.00 | 34.64 | C |
| ATOM | 104 | O | SER | A | 46 | 78.131 | 29.727 | 6.594 | 1.00 | 35.16 | O |
| ATOM | 105 | N | GLY | A | 47 | 80.033 | 28.645 | 6.168 | 1.00 | 36.14 | N |
| ATOM | 106 | CA | GLY | A | 47 | 79.552 | 27.513 | 6.960 | 1.00 | 37.77 | C |
| ATOM | 107 | C | GLY | A | 47 | 78.827 | 26.410 | 6.202 | 1.00 | 38.06 | C |
| ATOM | 108 | O | GLY | A | 47 | 78.803 | 25.261 | 6.665 | 1.00 | 38.85 | O |
| ATOM | 109 | N | GLY | A | 48 | 78.228 | 26.756 | 5.058 | 1.00 | 38.13 | N |
| ATOM | 110 | CA | GLY | A | 48 | 77.816 | 25.765 | 4.072 | 1.00 | 37.47 | C |
| ATOM | 111 | C | GLY | A | 48 | 79.007 | 25.489 | 3.163 | 1.00 | 37.20 | C |
| ATOM | 112 | O | GLY | A | 48 | 80.154 | 25.459 | 3.631 | 1.00 | 37.27 | O |
| ATOM | 113 | N | PHE | A | 49 | 78.757 | 25.324 | 1.865 | 1.00 | 36.60 | N |
| ATOM | 114 | CA | PHE | A | 49 | 79.845 | 25.099 | 0.905 | 1.00 | 36.12 | C |
| ATOM | 115 | CB | PHE | A | 49 | 79.322 | 24.532 | −0.421 | 1.00 | 36.73 | C |
| ATOM | 116 | CG | PHE | A | 49 | 78.733 | 23.153 | −0.310 | 1.00 | 39.10 | C |
| ATOM | 117 | CD1 | PHE | A | 49 | 77.363 | 22.960 | −0.454 | 1.00 | 40.06 | C |
| ATOM | 118 | CE1 | PHE | A | 49 | 76.806 | 21.681 | −0.357 | 1.00 | 42.14 | C |
| ATOM | 119 | CZ | PHE | A | 49 | 77.624 | 20.575 | −0.105 | 1.00 | 43.04 | C |
| ATOM | 120 | CE2 | PHE | A | 49 | 79.003 | 20.755 | 0.045 | 1.00 | 43.48 | C |
| ATOM | 121 | CD2 | PHE | A | 49 | 79.550 | 22.043 | −0.061 | 1.00 | 42.11 | C |
| ATOM | 122 | C | PHE | A | 49 | 80.702 | 26.339 | 0.614 | 1.00 | 34.83 | C |
| ATOM | 123 | O | PHE | A | 49 | 81.884 | 26.195 | 0.286 | 1.00 | 35.14 | O |
| ATOM | 124 | N | GLY | A | 50 | 80.109 | 27.536 | 0.717 | 1.00 | 32.72 | N |
| ATOM | 125 | CA | GLY | A | 50 | 80.770 | 28.772 | 0.303 | 1.00 | 30.18 | C |
| ATOM | 126 | C | GLY | A | 50 | 80.831 | 29.895 | 1.335 | 1.00 | 28.53 | C |
| ATOM | 127 | O | GLY | A | 50 | 80.161 | 29.832 | 2.367 | 1.00 | 28.21 | O |
| ATOM | 128 | N | SER | A | 51 | 81.676 | 30.895 | 1.061 | 1.00 | 26.40 | N |
| ATOM | 129 | CA | SER | A | 51 | 81.722 | 32.156 | 1.803 | 1.00 | 23.83 | C |
| ATOM | 130 | CB | SER | A | 51 | 83.157 | 32.509 | 2.190 | 1.00 | 24.19 | C |
| ATOM | 131 | OG | SER | A | 51 | 83.773 | 31.474 | 2.937 | 1.00 | 23.82 | O |
| ATOM | 132 | C | SER | A | 51 | 81.167 | 33.245 | 0.888 | 1.00 | 22.65 | C |
| ATOM | 133 | O | SER | A | 51 | 81.640 | 33.423 | −0.242 | 1.00 | 21.83 | O |
| ATOM | 134 | N | VAL | A | 52 | 80.150 | 33.948 | 1.369 | 1.00 | 20.93 | N |
| ATOM | 135 | CA | VAL | A | 52 | 79.427 | 34.917 | 0.568 | 1.00 | 20.09 | C |
| ATOM | 136 | CB | VAL | A | 52 | 77.917 | 34.574 | 0.518 | 1.00 | 19.63 | C |
| ATOM | 137 | CG1 | VAL | A | 52 | 77.182 | 35.536 | −0.391 | 1.00 | 19.68 | C |
| ATOM | 138 | CG2 | VAL | A | 52 | 77.705 | 33.133 | 0.035 | 1.00 | 19.27 | C |
| ATOM | 139 | C | VAL | A | 52 | 79.629 | 36.330 | 1.119 | 1.00 | 20.59 | C |
| ATOM | 140 | O | VAL | A | 52 | 79.406 | 36.576 | 2.309 | 1.00 | 19.74 | O |
| ATOM | 141 | N | TYR | A | 53 | 80.053 | 37.241 | 0.250 | 1.00 | 20.61 | N |
| ATOM | 142 | CA | TYR | A | 53 | 80.316 | 38.624 | 0.640 | 1.00 | 21.62 | C |
| ATOM | 143 | CB | TYR | A | 53 | 81.737 | 39.034 | 0.247 | 1.00 | 21.19 | C |
| ATOM | 144 | CG | TYR | A | 53 | 82.842 | 38.256 | 0.922 | 1.00 | 22.16 | C |
| ATOM | 145 | CD1 | TYR | A | 53 | 83.201 | 36.980 | 0.470 | 1.00 | 21.94 | C |
| ATOM | 146 | CE1 | TYR | A | 53 | 84.225 | 36.265 | 1.078 | 1.00 | 22.80 | C |
| ATOM | 147 | CZ | TYR | A | 53 | 84.921 | 36.830 | 2.146 | 1.00 | 23.82 | C |
| ATOM | 148 | OH | TYR | A | 53 | 85.937 | 36.114 | 2.736 | 1.00 | 23.97 | O |
| ATOM | 149 | CE2 | TYR | A | 53 | 84.597 | 38.099 | 2.614 | 1.00 | 23.07 | C |
| ATOM | 150 | CD2 | TYR | A | 53 | 83.559 | 38.810 | 1.994 | 1.00 | 23.01 | C |
| ATOM | 151 | C | TYR | A | 53 | 79.354 | 39.597 | −0.024 | 1.00 | 22.34 | C |
| ATOM | 152 | O | TYR | A | 53 | 78.888 | 39.373 | −1.153 | 1.00 | 22.35 | O |
| ATOM | 153 | N | SER | A | 54 | 79.066 | 40.681 | 0.680 | 1.00 | 23.48 | N |
| ATOM | 154 | CA | SER | A | 54 | 78.404 | 41.822 | 0.074 | 1.00 | 24.82 | C |
| ATOM | 155 | CB | SER | A | 54 | 77.980 | 42.840 | 1.140 | 1.00 | 25.09 | C |
| ATOM | 156 | OG | SER | A | 54 | 77.307 | 43.939 | 0.545 | 1.00 | 25.39 | O |
| ATOM | 157 | C | SER | A | 54 | 79.384 | 42.461 | −0.889 | 1.00 | 25.68 | C |
| ATOM | 158 | O | SER | A | 54 | 80.586 | 42.513 | −0.616 | 1.00 | 25.83 | O |
| ATOM | 159 | N | GLY | A | 55 | 78.878 | 42.932 | −2.023 | 1.00 | 26.75 | N |
| ATOM | 160 | CA | GLY | A | 55 | 79.720 | 43.609 | −2.991 | 1.00 | 27.76 | C |
| ATOM | 161 | C | GLY | A | 55 | 78.986 | 44.633 | −3.827 | 1.00 | 28.87 | C |
| ATOM | 162 | O | GLY | A | 55 | 77.762 | 44.772 | −3.737 | 1.00 | 28.62 | O |
| ATOM | 163 | N | ILE | A | 56 | 79.750 | 45.358 | −4.641 | 1.00 | 30.03 | N |
| ATOM | 164 | CA | ILE | A | 56 | 79.200 | 46.346 | −5.557 | 1.00 | 31.52 | C |
| ATOM | 165 | CB | ILE | A | 56 | 79.291 | 47.773 | −4.953 | 1.00 | 31.73 | C |
| ATOM | 166 | CG1 | ILE | A | 56 | 78.306 | 47.955 | −3.790 | 1.00 | 32.00 | C |
| ATOM | 167 | CD1 | ILE | A | 56 | 78.762 | 48.992 | −2.750 | 1.00 | 34.29 | C |
| ATOM | 168 | CG2 | ILE | A | 56 | 79.038 | 48.830 | −6.014 | 1.00 | 32.25 | C |
| ATOM | 169 | C | ILE | A | 56 | 79.927 | 46.274 | −6.901 | 1.00 | 32.27 | C |
| ATOM | 170 | O | ILE | A | 56 | 81.153 | 46.245 | −6.956 | 1.00 | 32.20 | O |
| ATOM | 171 | N | ARG | A | 57 | 79.147 | 46.225 | −7.976 | 1.00 | 33.45 | N |
| ATOM | 172 | CA | ARG | A | 57 | 79.664 | 46.308 | −9.332 | 1.00 | 34.77 | C |
| ATOM | 173 | CB | ARG | A | 57 | 78.574 | 45.902 | −10.319 | 1.00 | 34.53 | C |
| ATOM | 174 | CG | ARG | A | 57 | 79.075 | 45.541 | −11.692 | 1.00 | 35.85 | C |
| ATOM | 175 | CD | ARG | A | 57 | 78.037 | 45.746 | −12.766 | 1.00 | 37.31 | C |
| ATOM | 176 | NE | ARG | A | 57 | 77.459 | 44.488 | −13.210 | 1.00 | 38.72 | N |
| ATOM | 177 | CZ | ARG | A | 57 | 76.191 | 44.334 | −13.580 | 1.00 | 39.18 | C |
| ATOM | 178 | NH1 | ARG | A | 57 | 75.347 | 45.360 | −13.561 | 1.00 | 38.58 | N |
| ATOM | 179 | NH2 | ARG | A | 57 | 75.764 | 43.143 | −13.967 | 1.00 | 40.00 | N |
| ATOM | 180 | C | ARG | A | 57 | 80.134 | 47.740 | −9.604 | 1.00 | 35.42 | C |
| ATOM | 181 | O | ARG | A | 57 | 79.329 | 48.667 | −9.609 | 1.00 | 35.07 | O |
| ATOM | 182 | N | VAL | A | 58 | 81.438 | 47.901 | −9.822 | 1.00 | 36.96 | N |
| ATOM | 183 | CA | VAL | A | 58 | 82.069 | 49.221 | −9.962 | 1.00 | 38.44 | C |
| ATOM | 184 | CB | VAL | A | 58 | 83.626 | 49.118 | −10.083 | 1.00 | 38.53 | C |
| ATOM | 185 | CG1 | VAL | A | 58 | 84.270 | 50.494 | −10.251 | 1.00 | 38.65 | C |
| ATOM | 186 | CG2 | VAL | A | 58 | 84.226 | 48.422 | −8.863 | 1.00 | 38.97 | C |
| ATOM | 187 | C | VAL | A | 58 | 81.472 | 50.033 | −11.125 | 1.00 | 39.26 | C |
| ATOM | 188 | O | VAL | A | 58 | 81.243 | 51.238 | −10.989 | 1.00 | 39.41 | O |
| ATOM | 189 | N | SER | A | 59 | 81.194 | 49.357 | −12.239 | 1.00 | 40.32 | N |
| ATOM | 190 | CA | SER | A | 59 | 80.704 | 50.007 | −13.459 | 1.00 | 41.50 | C |
| ATOM | 191 | CB | SER | A | 59 | 80.627 | 49.012 | −14.625 | 1.00 | 41.60 | C |
| ATOM | 192 | OG | SER | A | 59 | 80.059 | 47.777 | −14.225 | 1.00 | 42.83 | O |
| ATOM | 193 | C | SER | A | 59 | 79.380 | 50.778 | −13.310 | 1.00 | 41.87 | C |
| ATOM | 194 | O | SER | A | 59 | 79.205 | 51.830 | −13.933 | 1.00 | 42.28 | O |
| ATOM | 195 | N | ASP | A | 60 | 78.463 | 50.269 | −12.488 | 1.00 | 42.04 | N |
| ATOM | 196 | CA | ASP | A | 60 | 77.147 | 50.897 | −12.330 | 1.00 | 41.97 | C |
| ATOM | 197 | CB | ASP | A | 60 | 76.118 | 50.187 | −13.223 | 1.00 | 42.37 | C |
| ATOM | 198 | CG | ASP | A | 60 | 75.881 | 48.739 | −12.810 | 1.00 | 43.55 | C |
| ATOM | 199 | OD1 | ASP | A | 60 | 76.449 | 48.309 | −11.781 | 1.00 | 44.32 | O |
| ATOM | 200 | OD2 | ASP | A | 60 | 75.142 | 47.959 | −13.451 | 1.00 | 43.86 | O |
| ATOM | 201 | C | ASP | A | 60 | 76.631 | 50.996 | −10.878 | 1.00 | 41.41 | C |
| ATOM | 202 | O | ASP | A | 60 | 75.479 | 51.372 | −10.657 | 1.00 | 41.59 | O |
| ATOM | 203 | N | ASN | A | 61 | 77.484 | 50.667 | −9.905 | 1.00 | 40.49 | N |
| ATOM | 204 | CA | ASN | A | 61 | 77.122 | 50.639 | −8.475 | 1.00 | 39.60 | C |
| ATOM | 205 | CB | ASN | A | 61 | 76.722 | 52.026 | −7.961 | 1.00 | 39.96 | C |
| ATOM | 206 | CG | ASN | A | 61 | 77.888 | 52.981 | −7.902 | 1.00 | 41.52 | C |
| ATOM | 207 | OD1 | ASN | A | 61 | 78.785 | 52.837 | −7.065 | 1.00 | 42.83 | O |
| ATOM | 208 | ND2 | ASN | A | 61 | 77.883 | 53.971 | −8.792 | 1.00 | 42.64 | N |
| ATOM | 209 | C | ASN | A | 61 | 76.058 | 49.615 | −8.056 | 1.00 | 38.30 | C |
| ATOM | 210 | O | ASN | A | 61 | 75.557 | 49.667 | −6.930 | 1.00 | 38.56 | O |
| ATOM | 211 | N | LEU | A | 62 | 75.724 | 48.685 | −8.947 | 1.00 | 36.52 | N |
| ATOM | 212 | CA | LEU | A | 62 | 74.768 | 47.623 | −8.623 | 1.00 | 34.93 | C |
| ATOM | 213 | CB | LEU | A | 62 | 74.519 | 46.720 | −9.832 | 1.00 | 35.10 | C |
| ATOM | 214 | CG | LEU | A | 62 | 73.421 | 45.662 | −9.712 | 1.00 | 35.38 | C |
| ATOM | 215 | CD1 | LEU | A | 62 | 72.047 | 46.269 | −9.961 | 1.00 | 36.89 | C |
| ATOM | 216 | CD2 | LEU | A | 62 | 73.679 | 44.527 | −10.677 | 1.00 | 35.30 | C |
| ATOM | 217 | C | LEU | A | 62 | 75.222 | 46.778 | −7.425 | 1.00 | 33.33 | C |
| ATOM | 218 | O | LEU | A | 62 | 76.351 | 46.288 | −7.404 | 1.00 | 32.86 | O |
| ATOM | 219 | N | PRO | A | 63 | 74.340 | 46.624 | −6.436 | 1.00 | 32.03 | N |
| ATOM | 220 | CA | PRO | A | 63 | 74.576 | 45.708 | −5.312 | 1.00 | 30.66 | C |
| ATOM | 221 | CB | PRO | A | 63 | 73.328 | 45.893 | −4.440 | 1.00 | 30.79 | C |
| ATOM | 222 | CG | PRO | A | 63 | 72.770 | 47.219 | −4.848 | 1.00 | 32.09 | C |
| ATOM | 223 | CD | PRO | A | 63 | 73.039 | 47.311 | −6.319 | 1.00 | 32.13 | C |
| ATOM | 224 | C | PRO | A | 63 | 74.657 | 44.263 | −5.804 | 1.00 | 28.94 | C |
| ATOM | 225 | O | PRO | A | 63 | 73.788 | 43.821 | −6.570 | 1.00 | 28.84 | O |
| ATOM | 226 | N | VAL | A | 64 | 75.705 | 43.554 | −5.393 | 1.00 | 26.63 | N |
| ATOM | 227 | CA | VAL | A | 64 | 75.862 | 42.135 | −5.723 | 1.00 | 24.40 | C |
| ATOM | 228 | CB | VAL | A | 64 | 76.903 | 41.905 | −6.870 | 1.00 | 24.56 | C |
| ATOM | 229 | CG1 | VAL | A | 64 | 76.430 | 42.528 | −8.195 | 1.00 | 23.44 | C |
| ATOM | 230 | CG2 | VAL | A | 64 | 78.292 | 42.436 | −6.471 | 1.00 | 23.53 | C |
| ATOM | 231 | C | VAL | A | 64 | 76.295 | 41.339 | −4.488 | 1.00 | 23.30 | C |
| ATOM | 232 | O | VAL | A | 64 | 76.650 | 41.922 | −3.451 | 1.00 | 22.79 | O |
| ATOM | 233 | N | ALA | A | 65 | 76.259 | 40.014 | −4.609 | 1.00 | 21.63 | N |
| ATOM | 234 | CA | ALA | A | 65 | 76.828 | 39.124 | −3.608 | 1.00 | 20.83 | C |
| ATOM | 235 | CB | ALA | A | 65 | 75.761 | 38.231 | −2.984 | 1.00 | 20.55 | C |
| ATOM | 236 | C | ALA | A | 65 | 77.892 | 38.290 | −4.281 | 1.00 | 20.04 | C |
| ATOM | 237 | O | ALA | A | 65 | 77.704 | 37.828 | −5.408 | 1.00 | 21.14 | O |
| ATOM | 238 | N | ILE | A | 66 | 79.015 | 38.111 | −3.600 | 1.00 | 18.86 | N |
| ATOM | 239 | CA | ILE | A | 66 | 80.165 | 37.439 | −4.186 | 1.00 | 17.57 | C |
| ATOM | 240 | CB | ILE | A | 66 | 81.422 | 38.346 | −4.117 | 1.00 | 17.68 | C |
| ATOM | 241 | CG1 | ILE | A | 66 | 81.148 | 39.723 | −4.747 | 1.00 | 18.56 | C |
| ATOM | 242 | CD1 | ILE | A | 66 | 82.220 | 40.772 | −4.424 | 1.00 | 19.55 | C |
| ATOM | 243 | CG2 | ILE | A | 66 | 82.602 | 37.668 | −4.775 | 1.00 | 16.32 | C |
| ATOM | 244 | C | ILE | A | 66 | 80.402 | 36.161 | −3.408 | 1.00 | 16.96 | C |
| ATOM | 245 | O | ILE | A | 66 | 80.775 | 36.206 | −2.227 | 1.00 | 16.17 | O |
| ATOM | 246 | N | LYS | A | 67 | 80.179 | 35.031 | −4.077 | 1.00 | 16.24 | N |
| ATOM | 247 | CA | LYS | A | 67 | 80.255 | 33.718 | −3.444 | 1.00 | 15.79 | C |
| ATOM | 248 | CB | LYS | A | 67 | 78.975 | 32.905 | −3.707 | 1.00 | 15.38 | C |
| ATOM | 249 | CG | LYS | A | 67 | 79.010 | 31.493 | −3.119 | 1.00 | 14.88 | C |
| ATOM | 250 | CD | LYS | A | 67 | 77.664 | 30.772 | −3.303 | 1.00 | 17.48 | C |
| ATOM | 251 | CE | LYS | A | 67 | 77.585 | 29.486 | −2.479 | 1.00 | 18.05 | C |
| ATOM | 252 | NZ | LYS | A | 67 | 76.184 | 28.951 | −2.470 | 1.00 | 18.14 | N |
| ATOM | 253 | C | LYS | A | 67 | 81.478 | 32.943 | −3.915 | 1.00 | 16.15 | C |
| ATOM | 254 | O | LYS | A | 67 | 81.667 | 32.705 | −5.122 | 1.00 | 15.33 | O |
| ATOM | 255 | N | HIS | A | 68 | 82.293 | 32.534 | −2.951 | 1.00 | 16.82 | N |
| ATOM | 256 | CA | HIS | A | 68 | 83.519 | 31.792 | −3.235 | 1.00 | 17.55 | C |
| ATOM | 257 | CB | HIS | A | 68 | 84.683 | 32.368 | −2.435 | 1.00 | 17.11 | C |
| ATOM | 258 | CG | HIS | A | 68 | 85.043 | 33.764 | −2.818 | 1.00 | 17.46 | C |
| ATOM | 259 | ND1 | HIS | A | 68 | 84.358 | 34.860 | −2.348 | 1.00 | 18.28 | N |
| ATOM | 260 | CE1 | HIS | A | 68 | 84.897 | 35.958 | −2.844 | 1.00 | 17.55 | C |
| ATOM | 261 | NE2 | HIS | A | 68 | 85.909 | 35.614 | −3.617 | 1.00 | 17.90 | N |
| ATOM | 262 | CD2 | HIS | A | 68 | 86.019 | 34.245 | −3.622 | 1.00 | 17.05 | C |
| ATOM | 263 | C | HIS | A | 68 | 83.319 | 30.353 | −2.829 | 1.00 | 18.47 | C |
| ATOM | 264 | O | HIS | A | 68 | 82.899 | 30.085 | −1.707 | 1.00 | 17.74 | O |
| ATOM | 265 | N | VAL | A | 69 | 83.628 | 29.434 | −3.735 | 1.00 | 19.87 | N |
| ATOM | 266 | CA | VAL | A | 69 | 83.538 | 28.016 | −3.441 | 1.00 | 21.97 | C |
| ATOM | 267 | CB | VAL | A | 69 | 82.386 | 27.316 | −4.229 | 1.00 | 22.09 | C |
| ATOM | 268 | CG1 | VAL | A | 69 | 82.270 | 25.863 | −3.809 | 1.00 | 22.01 | C |
| ATOM | 269 | CG2 | VAL | A | 69 | 81.049 | 28.011 | −3.992 | 1.00 | 22.34 | C |
| ATOM | 270 | C | VAL | A | 69 | 84.870 | 27.345 | −3.768 | 1.00 | 23.59 | C |
| ATOM | 271 | O | VAL | A | 69 | 85.331 | 27.388 | −4.903 | 1.00 | 23.28 | O |
| ATOM | 272 | N | GLU | A | 70 | 85.474 | 26.719 | −2.766 | 1.00 | 26.14 | N |
| ATOM | 273 | CA | GLU | A | 70 | 86.719 | 25.981 | −2.948 | 1.00 | 29.32 | C |
| ATOM | 274 | CB | GLU | A | 70 | 87.280 | 25.543 | −1.599 | 1.00 | 29.52 | C |
| ATOM | 275 | CG | GLU | A | 70 | 88.286 | 26.512 | −1.001 | 1.00 | 32.13 | C |
| ATOM | 276 | CD | GLU | A | 70 | 88.827 | 26.043 | 0.342 | 1.00 | 34.86 | C |
| ATOM | 277 | OE1 | GLU | A | 70 | 89.185 | 24.847 | 0.448 | 1.00 | 34.79 | O |
| ATOM | 278 | OE2 | GLU | A | 70 | 88.899 | 26.871 | 1.288 | 1.00 | 35.33 | O |
| ATOM | 279 | C | GLU | A | 70 | 86.486 | 24.760 | −3.834 | 1.00 | 31.18 | C |
| ATOM | 280 | O | GLU | A | 70 | 85.485 | 24.044 | −3.674 | 1.00 | 30.70 | O |
| ATOM | 281 | N | LYS | A | 71 | 87.402 | 24.540 | −4.774 | 1.00 | 33.73 | N |
| ATOM | 282 | CA | LYS | A | 71 | 87.292 | 23.422 | −5.718 | 1.00 | 36.85 | C |
| ATOM | 283 | CB | LYS | A | 71 | 88.426 | 23.459 | −6.734 | 1.00 | 36.40 | C |
| ATOM | 284 | CG | LYS | A | 71 | 88.228 | 24.487 | −7.822 | 1.00 | 35.73 | C |
| ATOM | 285 | CD | LYS | A | 71 | 89.373 | 24.457 | −8.814 | 1.00 | 35.72 | C |
| ATOM | 286 | CE | LYS | A | 71 | 89.168 | 25.490 | −9.898 | 1.00 | 35.77 | C |
| ATOM | 287 | NZ | LYS | A | 71 | 90.289 | 25.535 | −10.874 | 1.00 | 35.56 | N |
| ATOM | 288 | C | LYS | A | 71 | 87.206 | 22.046 | −5.047 | 1.00 | 39.35 | C |
| ATOM | 289 | O | LYS | A | 71 | 86.492 | 21.169 | −5.536 | 1.00 | 39.62 | O |
| ATOM | 290 | N | ASP | A | 72 | 87.904 | 21.872 | −3.922 | 1.00 | 42.63 | N |
| ATOM | 291 | CA | ASP | A | 72 | 87.873 | 20.615 | −3.161 | 1.00 | 46.03 | C |
| ATOM | 292 | CB | ASP | A | 72 | 89.021 | 20.560 | −2.145 | 1.00 | 46.33 | C |
| ATOM | 293 | CG | ASP | A | 72 | 90.396 | 20.511 | −2.811 | 1.00 | 48.21 | C |
| ATOM | 294 | OD1 | ASP | A | 72 | 90.519 | 19.935 | −3.918 | 1.00 | 49.79 | O |
| ATOM | 295 | OD2 | ASP | A | 72 | 91.418 | 21.025 | −2.300 | 1.00 | 50.39 | O |
| ATOM | 296 | C | ASP | A | 72 | 86.539 | 20.371 | −2.452 | 1.00 | 47.89 | C |
| ATOM | 297 | O | ASP | A | 72 | 86.138 | 19.221 | −2.253 | 1.00 | 48.61 | O |
| ATOM | 298 | N | ARG | A | 73 | 85.861 | 21.457 | −2.085 | 1.00 | 50.08 | N |
| ATOM | 299 | CA | ARG | A | 73 | 84.592 | 21.405 | −1.352 | 1.00 | 52.11 | C |
| ATOM | 300 | CB | ARG | A | 73 | 84.430 | 22.662 | −0.486 | 1.00 | 52.39 | C |
| ATOM | 301 | CG | ARG | A | 73 | 85.333 | 22.711 | 0.739 | 1.00 | 54.59 | C |
| ATOM | 302 | CD | ARG | A | 73 | 84.894 | 23.708 | 1.827 | 1.00 | 58.87 | C |
| ATOM | 303 | NE | ARG | A | 73 | 83.451 | 23.686 | 2.113 | 1.00 | 62.15 | N |
| ATOM | 304 | CZ | ARG | A | 73 | 82.792 | 22.682 | 2.708 | 1.00 | 63.76 | C |
| ATOM | 305 | NH1 | ARG | A | 73 | 83.427 | 21.579 | 3.094 | 1.00 | 64.18 | N |
| ATOM | 306 | NH2 | ARG | A | 73 | 81.484 | 22.779 | 2.917 | 1.00 | 63.67 | N |
| ATOM | 307 | C | ARG | A | 73 | 83.376 | 21.251 | −2.272 | 1.00 | 52.88 | C |
| ATOM | 308 | O | ARG | A | 73 | 82.246 | 21.100 | −1.793 | 1.00 | 52.85 | O |
| ATOM | 309 | N | ILE | A | 74 | 83.613 | 21.307 | −3.585 | 1.00 | 54.14 | N |
| ATOM | 310 | CA | ILE | A | 74 | 82.553 | 21.153 | −4.583 | 1.00 | 55.27 | C |
| ATOM | 311 | CB | ILE | A | 74 | 83.013 | 21.668 | −5.983 | 1.00 | 55.13 | C |
| ATOM | 312 | CG1 | ILE | A | 74 | 83.104 | 23.193 | −5.985 | 1.00 | 54.96 | C |
| ATOM | 313 | CD1 | ILE | A | 74 | 83.829 | 23.776 | −7.180 | 1.00 | 55.18 | C |
| ATOM | 314 | CG2 | ILE | A | 74 | 82.053 | 21.205 | −7.084 | 1.00 | 55.44 | C |
| ATOM | 315 | C | ILE | A | 74 | 82.107 | 19.691 | −4.638 | 1.00 | 56.17 | C |
| ATOM | 316 | O | ILE | A | 74 | 82.902 | 18.798 | −4.973 | 1.00 | 56.10 | O |
| ATOM | 317 | N | SER | A | 75 | 80.836 | 19.459 | −4.298 | 1.00 | 57.17 | N |
| ATOM | 318 | CA | SER | A | 75 | 80.287 | 18.101 | −4.234 | 1.00 | 58.19 | C |
| ATOM | 319 | CB | SER | A | 75 | 78.943 | 18.064 | −3.485 | 1.00 | 58.21 | C |
| ATOM | 320 | OG | SER | A | 75 | 78.112 | 19.161 | −3.831 | 1.00 | 58.80 | O |
| ATOM | 321 | C | SER | A | 75 | 80.178 | 17.482 | −5.629 | 1.00 | 58.59 | C |
| ATOM | 322 | O | SER | A | 75 | 80.890 | 16.520 | −5.939 | 1.00 | 58.89 | O |
| ATOM | 323 | N | ASP | A | 76 | 79.313 | 18.055 | −6.469 | 1.00 | 58.93 | N |
| ATOM | 324 | CA | ASP | A | 76 | 79.125 | 17.581 | −7.840 | 1.00 | 59.19 | C |
| ATOM | 325 | CB | ASP | A | 76 | 77.646 | 17.265 | −8.101 | 1.00 | 59.25 | C |
| ATOM | 326 | CG | ASP | A | 76 | 77.174 | 16.004 | −7.377 | 1.00 | 60.21 | C |
| ATOM | 327 | CD1 | ASP | A | 76 | 75.950 | 15.733 | −7.378 | 1.00 | 60.76 | O |
| ATOM | 328 | OD2 | ASP | A | 76 | 77.946 | 15.218 | −6.783 | 1.00 | 61.47 | O |
| ATOM | 329 | C | ASP | A | 76 | 79.655 | 18.576 | −8.875 | 1.00 | 59.17 | C |
| ATOM | 330 | O | ASP | A | 76 | 79.536 | 19.794 | −8.702 | 1.00 | 59.08 | O |
| ATOM | 331 | N | TRP | A | 77 | 80.245 | 18.043 | −9.943 | 1.00 | 59.21 | N |
| ATOM | 332 | CA | TRP | A | 77 | 80.737 | 18.850 | −11.059 | 1.00 | 59.31 | C |
| ATOM | 333 | CB | TRP | A | 77 | 82.186 | 18.497 | −11.399 | 1.00 | 58.89 | C |
| ATOM | 334 | CG | TRP | A | 77 | 83.207 | 18.816 | −10.338 | 1.00 | 57.61 | C |
| ATOM | 335 | CD1 | TRP | A | 77 | 83.449 | 18.112 | −9.191 | 1.00 | 56.71 | C |
| ATOM | 336 | NE1 | TRP | A | 77 | 84.469 | 18.695 | −8.480 | 1.00 | 56.23 | N |
| ATOM | 337 | CE2 | TRP | A | 77 | 84.921 | 19.793 | −9.166 | 1.00 | 55.87 | C |
| ATOM | 338 | CD2 | TRP | A | 77 | 84.149 | 19.899 | −10.345 | 1.00 | 55.83 | C |
| ATOM | 339 | CE3 | TRP | A | 77 | 84.416 | 20.957 | −11.226 | 1.00 | 54.75 | C |
| ATOM | 340 | CZ3 | TRP | A | 77 | 85.429 | 21.857 | −10.909 | 1.00 | 54.49 | C |
| ATOM | 341 | CH2 | TRP | A | 77 | 86.179 | 21.722 | −9.728 | 1.00 | 54.62 | C |
| ATOM | 342 | CZ2 | TRP | A | 77 | 85.942 | 20.700 | −8.846 | 1.00 | 54.88 | C |
| ATOM | 343 | C | TRP | A | 77 | 79.880 | 18.620 | −12.297 | 1.00 | 59.97 | C |
| ATOM | 344 | O | TRP | A | 77 | 79.309 | 17.539 | −12.479 | 1.00 | 59.95 | O |
| ATOM | 345 | N | GLY | A | 78 | 79.814 | 19.636 | −13.152 | 1.00 | 60.60 | N |
| ATOM | 346 | CA | GLY | A | 78 | 79.019 | 19.573 | −14.362 | 1.00 | 61.69 | C |
| ATOM | 347 | C | GLY | A | 78 | 79.663 | 20.261 | −15.546 | 1.00 | 62.62 | C |
| ATOM | 348 | O | GLY | A | 78 | 80.722 | 20.887 | −15.425 | 1.00 | 62.53 | O |
| ATOM | 349 | N | GLU | A | 79 | 79.016 | 20.127 | −16.700 | 1.00 | 63.55 | N |
| ATOM | 350 | CA | GLU | A | 79 | 79.470 | 20.764 | −17.932 | 1.00 | 64.55 | C |
| ATOM | 351 | CB | GLU | A | 79 | 79.841 | 19.724 | −19.007 | 1.00 | 64.74 | C |
| ATOM | 352 | CG | GLU | A | 79 | 78.740 | 18.730 | −19.386 | 1.00 | 65.56 | C |
| ATOM | 353 | CD | GLU | A | 79 | 78.780 | 18.319 | −20.857 | 1.00 | 67.08 | C |
| ATOM | 354 | OE1 | GLU | A | 79 | 79.892 | 18.197 | −21.428 | 1.00 | 67.41 | O |
| ATOM | 355 | OE2 | GLU | A | 79 | 77.693 | 18.111 | −21.446 | 1.00 | 66.84 | O |
| ATOM | 356 | C | GLU | A | 79 | 78.425 | 21.745 | −18.456 | 1.00 | 64.93 | C |
| ATOM | 357 | O | GLU | A | 79 | 77.218 | 21.485 | −18.388 | 1.00 | 64.82 | O |
| ATOM | 358 | N | LEU | A | 80 | 78.902 | 22.878 | −18.963 | 1.00 | 65.56 | N |
| ATOM | 359 | CA | LEU | A | 80 | 78.039 | 23.875 | −19.589 | 1.00 | 66.20 | C |
| ATOM | 360 | CB | LEU | A | 80 | 78.763 | 25.227 | −19.663 | 1.00 | 66.19 | C |
| ATOM | 361 | CG | LEU | A | 80 | 79.128 | 25.944 | −18.359 | 1.00 | 66.08 | C |
| ATOM | 362 | CD1 | LEU | A | 80 | 79.914 | 27.225 | −18.646 | 1.00 | 65.61 | C |
| ATOM | 363 | CD2 | LEU | A | 80 | 77.881 | 26.238 | −17.525 | 1.00 | 65.99 | C |
| ATOM | 364 | C | LEU | A | 80 | 77.662 | 23.402 | −20.996 | 1.00 | 66.59 | C |
| ATOM | 365 | O | LEU | A | 80 | 78.387 | 22.585 | −21.575 | 1.00 | 66.81 | O |
| ATOM | 366 | N | PRO | A | 81 | 76.539 | 23.885 | −21.547 | 1.00 | 66.88 | N |
| ATOM | 367 | CA | PRO | A | 81 | 76.220 | 23.634 | −22.963 | 1.00 | 66.94 | C |
| ATOM | 368 | CB | PRO | A | 81 | 75.035 | 24.573 | −23.226 | 1.00 | 67.02 | C |
| ATOM | 369 | CG | PRO | A | 81 | 74.363 | 24.703 | −21.892 | 1.00 | 67.04 | C |
| ATOM | 370 | CD | PRO | A | 81 | 75.477 | 24.665 | −20.877 | 1.00 | 66.98 | C |
| ATOM | 371 | C | PRO | A | 81 | 77.408 | 23.972 | −23.884 | 1.00 | 66.80 | C |
| ATOM | 372 | O | PRO | A | 81 | 77.505 | 23.438 | −24.990 | 1.00 | 66.90 | O |
| ATOM | 373 | N | ASN | A | 82 | 78.296 | 24.842 | −23.405 | 1.00 | 66.49 | N |
| ATOM | 374 | CA | ASN | A | 82 | 79.543 | 25.182 | −24.087 | 1.00 | 66.14 | C |
| ATOM | 375 | CB | ASN | A | 82 | 80.114 | 26.482 | −23.492 | 1.00 | 66.36 | C |
| ATOM | 376 | CG | ASN | A | 82 | 81.498 | 26.816 | −24.015 | 1.00 | 67.00 | C |
| ATOM | 377 | OD1 | ASN | A | 82 | 81.734 | 26.837 | −25.225 | 1.00 | 67.52 | O |
| ATOM | 378 | ND2 | ASN | A | 82 | 82.424 | 27.089 | −23.100 | 1.00 | 67.69 | N |
| ATOM | 379 | C | ASN | A | 82 | 80.576 | 24.044 | −24.035 | 1.00 | 65.50 | C |
| ATOM | 380 | O | ASN | A | 82 | 81.273 | 23.787 | −25.019 | 1.00 | 65.53 | O |
| ATOM | 381 | N | GLY | A | 83 | 80.664 | 23.369 | −22.888 | 1.00 | 64.74 | N |
| ATOM | 382 | CA | GLY | A | 83 | 81.614 | 22.284 | −22.683 | 1.00 | 63.59 | C |
| ATOM | 383 | C | GLY | A | 83 | 82.826 | 22.690 | −21.857 | 1.00 | 62.70 | C |
| ATOM | 384 | O | GLY | A | 83 | 83.967 | 22.600 | −22.326 | 1.00 | 62.98 | O |
| ATOM | 385 | N | THR | A | 84 | 82.571 | 23.149 | −20.632 | 1.00 | 61.37 | N |
| ATOM | 386 | CA | THR | A | 84 | 83.621 | 23.529 | −19.682 | 1.00 | 59.87 | C |
| ATOM | 387 | CB | THR | A | 84 | 83.742 | 25.067 | −19.576 | 1.00 | 60.05 | C |
| ATOM | 388 | OG1 | THR | A | 84 | 83.799 | 25.643 | −20.888 | 1.00 | 60.81 | O |
| ATOM | 389 | CG2 | THR | A | 84 | 85.080 | 25.468 | −18.954 | 1.00 | 60.25 | C |
| ATOM | 390 | C | THR | A | 84 | 83.309 | 22.938 | −18.310 | 1.00 | 58.33 | C |
| ATOM | 391 | O | THR | A | 84 | 82.139 | 22.816 | −17.930 | 1.00 | 58.41 | O |
| ATOM | 392 | N | ARG | A | 85 | 84.356 | 22.576 | −17.572 | 1.00 | 56.14 | N |
| ATOM | 393 | CA | ARG | A | 85 | 84.198 | 22.026 | −16.231 | 1.00 | 53.98 | C |
| ATOM | 394 | CB | ARG | A | 85 | 85.445 | 21.223 | −15.844 | 1.00 | 54.58 | C |
| ATOM | 395 | CG | ARG | A | 85 | 85.227 | 20.243 | −14.703 | 1.00 | 56.25 | C |
| ATOM | 396 | CD | ARG | A | 85 | 86.028 | 18.945 | −14.819 | 1.00 | 58.89 | C |
| ATOM | 397 | NE | ARG | A | 85 | 85.870 | 18.099 | −13.630 | 1.00 | 60.27 | N |
| ATOM | 398 | CZ | ARG | A | 85 | 84.879 | 17.226 | −13.445 | 1.00 | 60.85 | C |
| ATOM | 399 | NH1 | ARG | A | 85 | 83.933 | 17.065 | −14.370 | 1.00 | 61.04 | N |
| ATOM | 400 | NH2 | ARG | A | 85 | 84.834 | 16.506 | −12.329 | 1.00 | 60.48 | N |
| ATOM | 401 | C | ARG | A | 85 | 83.906 | 23.130 | −15.200 | 1.00 | 51.86 | C |
| ATOM | 402 | O | ARG | A | 85 | 84.789 | 23.931 | −14.865 | 1.00 | 51.74 | O |
| ATOM | 403 | N | VAL | A | 86 | 82.659 | 23.172 | −14.721 | 1.00 | 48.87 | N |
| ATOM | 404 | CA | VAL | A | 86 | 82.224 | 24.121 | −13.680 | 1.00 | 45.97 | C |
| ATOM | 405 | CB | VAL | A | 86 | 81.335 | 25.276 | −14.251 | 1.00 | 46.05 | C |
| ATOM | 406 | CG1 | VAL | A | 86 | 82.074 | 26.064 | −15.322 | 1.00 | 46.41 | C |
| ATOM | 407 | CG2 | VAL | A | 86 | 80.008 | 24.755 | −14.781 | 1.00 | 45.90 | C |
| ATOM | 408 | C | VAL | A | 86 | 81.462 | 23.409 | −12.553 | 1.00 | 43.56 | C |
| ATOM | 409 | O | VAL | A | 86 | 80.984 | 22.292 | −12.750 | 1.00 | 43.24 | O |
| ATOM | 410 | N | PRO | A | 87 | 81.345 | 24.040 | −11.380 | 1.00 | 41.11 | N |
| ATOM | 411 | CA | PRO | A | 87 | 80.494 | 23.495 | −10.314 | 1.00 | 39.00 | C |
| ATOM | 412 | CB | PRO | A | 87 | 80.654 | 24.499 | −9.158 | 1.00 | 39.06 | C |
| ATOM | 413 | CG | PRO | A | 87 | 81.251 | 25.719 | −9.759 | 1.00 | 40.31 | C |
| ATOM | 414 | CD | PRO | A | 87 | 82.015 | 25.287 | −10.966 | 1.00 | 40.95 | C |
| ATOM | 415 | C | PRO | A | 87 | 79.037 | 23.413 | −10.755 | 1.00 | 36.73 | C |
| ATOM | 416 | O | PRO | A | 87 | 78.567 | 24.258 | −11.535 | 1.00 | 35.64 | O |
| ATOM | 417 | N | MET | A | 88 | 78.343 | 22.391 | −10.255 | 1.00 | 34.56 | N |
| ATOM | 418 | CA | MET | A | 88 | 76.936 | 22.171 | −10.564 | 1.00 | 32.47 | C |
| ATOM | 419 | CB | MET | A | 88 | 76.408 | 20.950 | −9.799 | 1.00 | 33.23 | C |
| ATOM | 420 | CG | MET | A | 88 | 75.047 | 20.407 | −10.263 | 1.00 | 36.09 | C |
| ATOM | 421 | SD | MET | A | 88 | 74.917 | 19.957 | −12.033 | 1.00 | 43.02 | S |
| ATOM | 422 | CE | MET | A | 88 | 76.260 | 18.799 | −12.218 | 1.00 | 41.30 | C |
| ATOM | 423 | C | MET | A | 88 | 76.110 | 23.423 | −10.274 | 1.00 | 30.24 | C |
| ATOM | 424 | O | MET | A | 88 | 75.169 | 23.717 | −11.006 | 1.00 | 29.10 | O |
| ATOM | 425 | N | GLU | A | 89 | 76.487 | 24.169 | −9.231 | 1.00 | 28.21 | N |
| ATOM | 426 | CA | GLU | A | 89 | 75.773 | 25.392 | −8.843 | 1.00 | 26.55 | C |
| ATOM | 427 | CB | GLU | A | 89 | 76.393 | 26.043 | −7.576 | 1.00 | 26.83 | C |
| ATOM | 428 | CG | GLU | A | 89 | 75.711 | 27.347 | −7.134 | 1.00 | 27.21 | C |
| ATOM | 429 | CD | GLU | A | 89 | 75.939 | 27.733 | −5.670 | 1.00 | 29.78 | C |
| ATOM | 430 | OE1 | GLU | A | 89 | 76.956 | 27.292 | −5.073 | 1.00 | 29.01 | O |
| ATOM | 431 | OE2 | GLU | A | 89 | 75.080 | 28.483 | −5.118 | 1.00 | 29.29 | O |
| ATOM | 432 | C | GLU | A | 89 | 75.669 | 26.392 | −10.000 | 1.00 | 25.41 | C |
| ATOM | 433 | O | GLU | A | 89 | 74.609 | 26.988 | −10.223 | 1.00 | 25.07 | O |
| ATOM | 434 | N | VAL | A | 90 | 76.761 | 26.566 | −10.744 | 1.00 | 24.25 | N |
| ATOM | 435 | CA | VAL | A | 90 | 76.747 | 27.435 | −11.927 | 1.00 | 23.34 | C |
| ATOM | 436 | CB | VAL | A | 90 | 78.193 | 27.717 | −12.452 | 1.00 | 23.97 | C |
| ATOM | 437 | CG1 | VAL | A | 90 | 78.178 | 28.460 | −13.796 | 1.00 | 23.00 | C |
| ATOM | 438 | CG2 | VAL | A | 90 | 78.989 | 28.530 | −11.411 | 1.00 | 23.11 | C |
| ATOM | 439 | C | VAL | A | 90 | 75.822 | 26.881 | −13.020 | 1.00 | 22.89 | C |
| ATOM | 440 | O | VAL | A | 90 | 75.002 | 27.622 | −13.570 | 1.00 | 22.78 | O |
| ATOM | 441 | N | VAL | A | 91 | 75.926 | 25.579 | −13.305 | 1.00 | 22.51 | N |
| ATOM | 442 | CA | VAL | A | 91 | 75.048 | 24.917 | −14.293 | 1.00 | 22.19 | C |
| ATOM | 443 | CB | VAL | A | 91 | 75.316 | 23.382 | −14.399 | 1.00 | 22.50 | C |
| ATOM | 444 | CG1 | VAL | A | 91 | 74.265 | 22.688 | −15.314 | 1.00 | 22.61 | C |
| ATOM | 445 | CG2 | VAL | A | 91 | 76.688 | 23.116 | −14.934 | 1.00 | 22.68 | C |
| ATOM | 446 | C | VAL | A | 91 | 73.569 | 25.143 | −13.965 | 1.00 | 21.66 | C |
| ATOM | 447 | O | VAL | A | 91 | 72.783 | 25.594 | −14.807 | 1.00 | 20.36 | O |
| ATOM | 448 | N | LEU | A | 92 | 73.215 | 24.856 | −12.715 | 1.00 | 21.61 | N |
| ATOM | 449 | CA | LEU | A | 92 | 71.833 | 24.963 | −12.256 | 1.00 | 21.57 | C |
| ATOM | 450 | CB | LEU | A | 92 | 71.682 | 24.326 | −10.873 | 1.00 | 21.14 | C |
| ATOM | 451 | CG | LEU | A | 92 | 72.112 | 22.854 | −10.778 | 1.00 | 21.50 | C |
| ATOM | 452 | CD1 | LEU | A | 92 | 71.945 | 22.365 | −9.349 | 1.00 | 20.58 | C |
| ATOM | 453 | CD2 | LEU | A | 92 | 71.378 | 21.928 | −11.767 | 1.00 | 18.41 | C |
| ATOM | 454 | C | LEU | A | 92 | 71.331 | 26.408 | −12.255 | 1.00 | 21.79 | C |
| ATOM | 455 | O | LEU | A | 92 | 70.212 | 26.671 | −12.706 | 1.00 | 21.50 | O |
| ATOM | 456 | N | LEU | A | 93 | 72.159 | 27.332 | −11.764 | 1.00 | 22.36 | N |
| ATOM | 457 | CA | LEU | A | 93 | 71.808 | 28.755 | −11.756 | 1.00 | 23.34 | C |
| ATOM | 458 | CB | LEU | A | 93 | 72.861 | 29.584 | −11.018 | 1.00 | 23.50 | C |
| ATOM | 459 | CG | LEU | A | 93 | 72.714 | 29.608 | −9.482 | 1.00 | 24.22 | C |
| ATOM | 460 | CD1 | LEU | A | 93 | 73.985 | 30.131 | −8.852 | 1.00 | 23.74 | C |
| ATOM | 461 | CD2 | LEU | A | 93 | 71.493 | 30.431 | −9.048 | 1.00 | 22.10 | C |
| ATOM | 462 | C | LEU | A | 93 | 71.594 | 29.301 | −13.162 | 1.00 | 23.97 | C |
| ATOM | 463 | O | LEU | A | 93 | 70.642 | 30.037 | −13.409 | 1.00 | 23.67 | O |
| ATOM | 464 | N | LYS | A | 94 | 72.468 | 28.922 | −14.088 | 1.00 | 24.87 | N |
| ATOM | 465 | CA | LYS | A | 94 | 72.265 | 29.290 | −15.491 | 1.00 | 26.26 | C |
| ATOM | 466 | CB | LYS | A | 94 | 73.448 | 28.847 | −16.356 | 1.00 | 26.36 | C |
| ATOM | 467 | CG | LYS | A | 94 | 74.664 | 29.745 | −16.166 | 1.00 | 29.54 | C |
| ATOM | 468 | CD | LYS | A | 94 | 75.932 | 29.132 | −16.750 | 1.00 | 34.13 | C |
| ATOM | 469 | CE | LYS | A | 94 | 76.210 | 29.633 | −18.167 | 1.00 | 36.90 | C |
| ATOM | 470 | NZ | LYS | A | 94 | 76.658 | 31.059 | −18.181 | 1.00 | 39.00 | N |
| ATOM | 471 | C | LYS | A | 94 | 70.946 | 28.761 | −16.045 | 1.00 | 26.31 | C |
| ATOM | 472 | O | LYS | A | 94 | 70.240 | 29.481 | −16.756 | 1.00 | 26.21 | O |
| ATOM | 473 | N | LYS | A | 95 | 70.610 | 27.515 | −15.712 | 1.00 | 26.64 | N |
| ATOM | 474 | CA | LYS | A | 95 | 69.356 | 26.916 | −16.172 | 1.00 | 28.04 | C |
| ATOM | 475 | CB | LYS | A | 95 | 69.295 | 25.427 | −15.824 | 1.00 | 27.38 | C |
| ATOM | 476 | CG | LYS | A | 95 | 70.096 | 24.557 | −16.777 | 1.00 | 28.63 | C |
| ATOM | 477 | CD | LYS | A | 95 | 70.218 | 23.114 | −16.294 | 1.00 | 29.42 | C |
| ATOM | 478 | CE | LYS | A | 95 | 68.891 | 22.361 | −16.392 | 1.00 | 30.74 | C |
| ATOM | 479 | NZ | LYS | A | 95 | 68.513 | 22.039 | −17.803 | 1.00 | 31.02 | N |
| ATOM | 480 | C | LYS | A | 95 | 68.096 | 27.657 | −15.674 | 1.00 | 28.66 | C |
| ATOM | 481 | O | LYS | A | 95 | 67.088 | 27.707 | −16.379 | 1.00 | 28.72 | O |
| ATOM | 482 | N | VAL | A | 96 | 68.167 | 28.239 | −14.480 | 1.00 | 30.05 | N |
| ATOM | 483 | CA | VAL | A | 96 | 67.017 | 28.940 | −13.899 | 1.00 | 31.67 | C |
| ATOM | 484 | CB | VAL | A | 96 | 66.818 | 28.631 | −12.383 | 1.00 | 31.32 | C |
| ATOM | 485 | CG1 | VAL | A | 96 | 66.594 | 27.139 | −12.159 | 1.00 | 30.44 | C |
| ATOM | 486 | CG2 | VAL | A | 96 | 67.978 | 29.149 | −11.546 | 1.00 | 29.93 | C |
| ATOM | 487 | C | VAL | A | 96 | 66.997 | 30.458 | −14.119 | 1.00 | 33.63 | C |
| ATOM | 488 | O | VAL | A | 96 | 65.999 | 31.112 | −13.783 | 1.00 | 33.80 | O |
| ATOM | 489 | N | SER | A | 97 | 68.074 | 31.018 | −14.676 | 1.00 | 35.26 | N |
| ATOM | 490 | CA | SER | A | 97 | 68.109 | 32.455 | −14.979 | 1.00 | 37.17 | C |
| ATOM | 491 | CB | SER | A | 97 | 69.490 | 32.907 | −15.483 | 1.00 | 37.37 | C |
| ATOM | 492 | OG | SER | A | 97 | 69.844 | 32.265 | −16.699 | 1.00 | 38.96 | O |
| ATOM | 493 | C | SER | A | 97 | 67.009 | 32.865 | −15.962 | 1.00 | 38.05 | C |
| ATOM | 494 | O | SER | A | 97 | 66.797 | 32.223 | −16.996 | 1.00 | 38.07 | O |
| ATOM | 495 | N | SER | A | 98 | 66.302 | 33.934 | −15.603 | 1.00 | 39.50 | N |
| ATOM | 496 | CA | SER | A | 98 | 65.224 | 34.521 | −16.409 | 1.00 | 40.49 | C |
| ATOM | 497 | CB | SER | A | 98 | 64.109 | 33.499 | −16.685 | 1.00 | 40.48 | C |
| ATOM | 498 | OG | SER | A | 98 | 63.105 | 33.547 | −15.681 | 1.00 | 41.42 | O |
| ATOM | 499 | C | SER | A | 98 | 64.671 | 35.738 | −15.656 | 1.00 | 40.82 | C |
| ATOM | 500 | O | SER | A | 98 | 65.177 | 36.091 | −14.582 | 1.00 | 41.27 | O |
| ATOM | 501 | N | GLY | A | 99 | 63.632 | 36.364 | −16.210 | 1.00 | 40.95 | N |
| ATOM | 502 | CA | GLY | A | 99 | 63.015 | 37.535 | −15.602 | 1.00 | 40.62 | C |
| ATOM | 503 | C | GLY | A | 99 | 62.281 | 37.294 | −14.283 | 1.00 | 40.18 | C |
| ATOM | 504 | O | GLY | A | 99 | 61.912 | 38.263 | −13.600 | 1.00 | 40.34 | O |
| ATOM | 505 | N | PHE | A | 100 | 62.056 | 36.019 | −13.942 | 1.00 | 39.30 | N |
| ATOM | 506 | CA | PHE | A | 100 | 61.391 | 35.628 | −12.694 | 1.00 | 38.13 | C |
| ATOM | 507 | CB | PHE | A | 100 | 61.038 | 34.135 | −12.709 | 1.00 | 38.35 | C |
| ATOM | 508 | CG | PHE | A | 100 | 60.296 | 33.656 | −11.471 | 1.00 | 37.90 | C |
| ATOM | 509 | CD1 | PHE | A | 100 | 59.100 | 34.258 | −11.069 | 1.00 | 36.99 | C |
| ATOM | 510 | CE1 | PHE | A | 100 | 58.411 | 33.801 | −9.924 | 1.00 | 37.18 | C |
| ATOM | 511 | CZ | PHE | A | 100 | 58.922 | 32.727 | −9.177 | 1.00 | 35.96 | C |
| ATOM | 512 | CE2 | PHE | A | 100 | 60.108 | 32.116 | −9.573 | 1.00 | 35.95 | C |
| ATOM | 513 | CD2 | PHE | A | 100 | 60.792 | 32.585 | −10.718 | 1.00 | 37.98 | C |
| ATOM | 514 | C | PHE | A | 100 | 62.266 | 35.944 | −11.491 | 1.00 | 37.35 | C |
| ATOM | 515 | O | PHE | A | 100 | 63.365 | 35.409 | −11.354 | 1.00 | 37.45 | O |
| ATOM | 516 | N | SER | A | 101 | 61.768 | 36.814 | −10.620 | 1.00 | 36.17 | N |
| ATOM | 517 | CA | SER | A | 101 | 62.534 | 37.263 | −9.468 | 1.00 | 35.23 | C |
| ATOM | 518 | CB | SER | A | 101 | 62.048 | 38.647 | −9.003 | 1.00 | 35.81 | C |
| ATOM | 519 | OG | SER | A | 101 | 60.697 | 38.612 | −8.570 | 1.00 | 37.04 | O |
| ATOM | 520 | C | SER | A | 101 | 62.571 | 36.280 | −8.291 | 1.00 | 33.45 | C |
| ATOM | 521 | O | SER | A | 101 | 63.260 | 36.544 | −7.295 | 1.00 | 33.93 | O |
| ATOM | 522 | N | GLY | A | 102 | 61.856 | 35.157 | −8.402 | 1.00 | 31.13 | N |
| ATOM | 523 | CA | GLY | A | 102 | 61.760 | 34.190 | −7.310 | 1.00 | 28.02 | C |
| ATOM | 524 | C | GLY | A | 102 | 63.026 | 33.377 | −7.066 | 1.00 | 26.06 | C |
| ATOM | 525 | O | GLY | A | 102 | 63.183 | 32.736 | −6.040 | 1.00 | 24.79 | O |
| ATOM | 526 | N | VAL | A | 103 | 63.936 | 33.396 | −8.030 | 1.00 | 25.16 | N |
| ATOM | 527 | CA | VAL | A | 103 | 65.213 | 32.726 | −7.877 | 1.00 | 24.40 | C |
| ATOM | 528 | CB | VAL | A | 103 | 65.377 | 31.540 | −8.863 | 1.00 | 24.34 | C |
| ATOM | 529 | CG1 | VAL | A | 103 | 66.675 | 30.797 | −8.585 | 1.00 | 25.25 | C |
| ATOM | 530 | CG2 | VAL | A | 103 | 64.214 | 30.567 | −8.737 | 1.00 | 23.66 | C |
| ATOM | 531 | C | VAL | A | 103 | 66.300 | 33.759 | −8.104 | 1.00 | 24.50 | C |
| ATOM | 532 | O | VAL | A | 103 | 66.217 | 34.566 | −9.040 | 1.00 | 24.27 | O |
| ATOM | 533 | N | ILE | A | 104 | 67.303 | 33.744 | −7.232 | 1.00 | 23.78 | N |
| ATOM | 534 | CA | ILE | A | 104 | 68.477 | 34.576 | −7.383 | 1.00 | 24.18 | C |
| ATOM | 535 | CB | ILE | A | 104 | 69.526 | 34.185 | −6.324 | 1.00 | 24.30 | C |
| ATOM | 536 | CG1 | ILE | A | 104 | 70.384 | 35.394 | −5.954 | 1.00 | 23.11 | C |
| ATOM | 537 | CD1 | ILE | A | 104 | 69.581 | 36.449 | −5.162 | 1.00 | 22.00 | C |
| ATOM | 538 | CG2 | ILE | A | 104 | 70.327 | 32.934 | −6.766 | 1.00 | 23.91 | C |
| ATOM | 539 | C | ILE | A | 104 | 69.083 | 34.483 | −8.789 | 1.00 | 24.77 | C |
| ATOM | 540 | O | ILE | A | 104 | 69.188 | 33.403 | −9.366 | 1.00 | 25.02 | O |
| ATOM | 541 | N | ARG | A | 105 | 69.479 | 35.619 | −9.337 | 1.00 | 25.08 | N |
| ATOM | 542 | CA | ARG | A | 105 | 70.070 | 35.622 | −10.667 | 1.00 | 26.17 | C |
| ATOM | 543 | CB | ARG | A | 105 | 69.566 | 36.833 | −11.454 | 1.00 | 27.31 | C |
| ATOM | 544 | CG | ARG | A | 105 | 70.349 | 37.173 | −12.714 | 1.00 | 32.33 | C |
| ATOM | 545 | CD | ARG | A | 105 | 69.728 | 38.311 | −13.536 | 1.00 | 39.80 | C |
| ATOM | 546 | NE | ARG | A | 105 | 68.331 | 38.040 | −13.891 | 1.00 | 45.22 | N |
| ATOM | 547 | CZ | ARG | A | 105 | 67.573 | 38.838 | −14.646 | 1.00 | 47.93 | C |
| ATOM | 548 | NH1 | ARG | A | 105 | 68.062 | 39.976 | −15.139 | 1.00 | 48.73 | N |
| ATOM | 549 | NH2 | ARG | A | 105 | 66.319 | 38.498 | −14.908 | 1.00 | 48.97 | N |
| ATOM | 550 | C | ARG | A | 105 | 71.593 | 35.590 | −10.594 | 1.00 | 25.19 | C |
| ATOM | 551 | O | ARG | A | 105 | 72.211 | 36.402 | −9.885 | 1.00 | 24.65 | O |
| ATOM | 552 | N | LEU | A | 106 | 72.188 | 34.634 | −11.304 | 1.00 | 24.85 | N |
| ATOM | 553 | CA | LEU | A | 106 | 73.642 | 34.609 | −11.499 | 1.00 | 24.90 | C |
| ATOM | 554 | CB | LEU | A | 106 | 74.136 | 33.223 | −11.918 | 1.00 | 24.41 | C |
| ATOM | 555 | CG | LEU | A | 106 | 75.651 | 33.073 | −12.127 | 1.00 | 24.89 | C |
| ATOM | 556 | CD1 | LEU | A | 106 | 76.449 | 33.148 | −10.796 | 1.00 | 24.67 | C |
| ATOM | 557 | CD2 | LEU | A | 106 | 75.961 | 31.790 | −12.871 | 1.00 | 23.97 | C |
| ATOM | 558 | C | LEU | A | 106 | 74.004 | 35.639 | −12.554 | 1.00 | 25.16 | C |
| ATOM | 559 | O | LEU | A | 106 | 73.536 | 35.565 | −13.695 | 1.00 | 25.41 | O |
| ATOM | 560 | N | LEU | A | 107 | 74.825 | 36.604 | −12.163 | 1.00 | 25.22 | N |
| ATOM | 561 | CA | LEU | A | 107 | 75.217 | 37.703 | −13.046 | 1.00 | 25.72 | C |
| ATOM | 562 | CB | LEU | A | 107 | 75.427 | 38.991 | −12.240 | 1.00 | 25.54 | C |
| ATOM | 563 | CG | LEU | A | 107 | 74.167 | 39.501 | −11.524 | 1.00 | 25.95 | C |
| ATOM | 564 | CD1 | LEU | A | 107 | 74.478 | 40.685 | −10.620 | 1.00 | 25.00 | C |
| ATOM | 565 | CD2 | LEU | A | 107 | 73.067 | 39.868 | −12.525 | 1.00 | 27.51 | C |
| ATOM | 566 | C | LEU | A | 107 | 76.465 | 37.363 | −13.847 | 1.00 | 25.66 | C |
| ATOM | 567 | O | LEU | A | 107 | 76.553 | 37.678 | −15.040 | 1.00 | 25.68 | O |
| ATOM | 568 | N | ASP | A | 108 | 77.420 | 36.717 | −13.177 | 1.00 | 25.47 | N |
| ATOM | 569 | CA | ASP | A | 108 | 78.699 | 36.333 | −13.762 | 1.00 | 25.47 | C |
| ATOM | 570 | CB | ASP | A | 108 | 79.624 | 37.557 | −13.872 | 1.00 | 25.78 | C |
| ATOM | 571 | CG | ASP | A | 108 | 80.569 | 37.485 | −15.071 | 1.00 | 27.00 | C |
| ATOM | 572 | OD1 | ASP | A | 108 | 80.828 | 36.385 | −15.610 | 1.00 | 27.45 | O |
| ATOM | 573 | OD2 | ASP | A | 108 | 81.111 | 38.501 | −15.537 | 1.00 | 29.70 | O |
| ATOM | 574 | C | ASP | A | 108 | 79.358 | 35.308 | −12.856 | 1.00 | 25.21 | C |
| ATOM | 575 | O | ASP | A | 108 | 78.938 | 35.119 | −11.711 | 1.00 | 23.96 | O |
| ATOM | 576 | N | TRP | A | 109 | 80.405 | 34.669 | −13.369 | 1.00 | 25.09 | N |
| ATOM | 577 | CA | TRP | A | 109 | 81.235 | 33.785 | −12.565 | 1.00 | 25.83 | C |
| ATOM | 578 | CB | TRP | A | 109 | 80.668 | 32.360 | −12.565 | 1.00 | 26.03 | C |
| ATOM | 579 | CG | TRP | A | 109 | 80.690 | 31.729 | −13.918 | 1.00 | 27.82 | C |
| ATOM | 580 | CD1 | TRP | A | 109 | 79.727 | 31.818 | −14.883 | 1.00 | 28.39 | C |
| ATOM | 581 | NE1 | TRP | A | 109 | 80.102 | 31.102 | −15.995 | 1.00 | 30.13 | N |
| ATOM | 582 | CE2 | TRP | A | 109 | 81.332 | 30.539 | −15.770 | 1.00 | 30.67 | C |
| ATOM | 583 | CD2 | TRP | A | 109 | 81.732 | 30.914 | −14.465 | 1.00 | 29.83 | C |
| ATOM | 584 | CE3 | TRP | A | 109 | 82.970 | 30.460 | −13.987 | 1.00 | 30.76 | C |
| ATOM | 585 | CZ3 | TRP | A | 109 | 83.758 | 29.664 | −14.809 | 1.00 | 32.48 | C |
| ATOM | 586 | CH2 | TRP | A | 109 | 83.334 | 29.313 | −16.107 | 1.00 | 33.06 | C |
| ATOM | 587 | CZ2 | TRP | A | 109 | 82.126 | 29.739 | −16.602 | 1.00 | 32.39 | C |
| ATOM | 588 | C | TRP | A | 109 | 82.689 | 33.808 | −13.045 | 1.00 | 26.10 | C |
| ATOM | 589 | O | TRP | A | 109 | 82.973 | 34.170 | −14.191 | 1.00 | 25.61 | O |
| ATOM | 590 | N | PHE | A | 110 | 83.599 | 33.425 | −12.155 | 1.00 | 26.36 | N |
| ATOM | 591 | CA | PHE | A | 110 | 85.028 | 33.407 | −12.449 | 1.00 | 26.90 | C |
| ATOM | 592 | CB | PHE | A | 110 | 85.734 | 34.607 | −11.796 | 1.00 | 27.07 | C |
| ATOM | 593 | CG | PHE | A | 110 | 85.249 | 35.945 | −12.285 | 1.00 | 28.66 | C |
| ATOM | 594 | CD1 | PHE | A | 110 | 85.909 | 36.602 | −13.330 | 1.00 | 30.61 | C |
| ATOM | 595 | CE1 | PHE | A | 110 | 85.464 | 37.851 | −13.785 | 1.00 | 31.43 | C |
| ATOM | 596 | CZ | PHE | A | 110 | 84.344 | 38.446 | −13.195 | 1.00 | 31.46 | C |
| ATOM | 597 | CE2 | PHE | A | 110 | 83.679 | 37.795 | −12.153 | 1.00 | 30.52 | C |
| ATOM | 598 | CD2 | PHE | A | 110 | 84.140 | 36.556 | −11.701 | 1.00 | 28.63 | C |
| ATOM | 599 | C | PHE | A | 110 | 85.646 | 32.117 | −11.924 | 1.00 | 27.02 | C |
| ATOM | 600 | O | PHE | A | 110 | 85.227 | 31.591 | −10.879 | 1.00 | 26.50 | O |
| ATOM | 601 | N | GLU | A | 111 | 86.638 | 31.614 | −12.655 | 1.00 | 26.99 | N |
| ATOM | 602 | CA | GLU | A | 111 | 87.454 | 30.500 | −12.201 | 1.00 | 27.50 | C |
| ATOM | 603 | CB | GLU | A | 111 | 87.683 | 29.476 | −13.323 | 1.00 | 27.99 | C |
| ATOM | 604 | CG | GLU | A | 111 | 88.309 | 28.179 | −12.828 | 1.00 | 28.36 | C |
| ATOM | 605 | CD | GLU | A | 111 | 88.468 | 27.116 | −13.894 | 1.00 | 29.75 | C |
| ATOM | 606 | OE1 | GLU | A | 111 | 87.864 | 27.215 | −14.989 | 1.00 | 31.47 | O |
| ATOM | 607 | OE2 | GLU | A | 111 | 89.206 | 26.154 | −13.622 | 1.00 | 30.33 | O |
| ATOM | 608 | C | GLU | A | 111 | 88.796 | 31.023 | −11.696 | 1.00 | 27.98 | C |
| ATOM | 609 | O | GLU | A | 111 | 89.415 | 31.891 | −12.310 | 1.00 | 28.26 | O |
| ATOM | 610 | N | ARG | A | 112 | 89.225 | 30.490 | −10.560 | 1.00 | 28.31 | N |
| ATOM | 611 | CA | ARG | A | 112 | 90.541 | 30.760 | −10.004 | 1.00 | 28.18 | C |
| ATOM | 612 | CB | ARG | A | 112 | 90.403 | 31.378 | −8.614 | 1.00 | 28.37 | C |
| ATOM | 613 | CG | ARG | A | 112 | 90.263 | 32.883 | −8.622 | 1.00 | 27.57 | C |
| ATOM | 614 | CD | ARG | A | 112 | 89.828 | 33.452 | −7.293 | 1.00 | 27.27 | C |
| ATOM | 615 | NE | ARG | A | 112 | 89.976 | 34.899 | −7.282 | 1.00 | 26.93 | N |
| ATOM | 616 | CZ | ARG | A | 112 | 89.758 | 35.671 | −6.233 | 1.00 | 27.51 | C |
| ATOM | 617 | NH1 | ARG | A | 112 | 89.360 | 35.146 | −5.079 | 1.00 | 28.76 | N |
| ATOM | 618 | NH2 | ARG | A | 112 | 89.932 | 36.979 | −6.339 | 1.00 | 26.92 | N |
| ATOM | 619 | C | ARG | A | 112 | 91.255 | 29.413 | −9.933 | 1.00 | 28.60 | C |
| ATOM | 620 | O | ARG | A | 112 | 90.627 | 28.379 | −10.197 | 1.00 | 28.00 | O |
| ATOM | 621 | N | PRO | A | 113 | 92.556 | 29.402 | −9.616 | 1.00 | 28.77 | N |
| ATOM | 622 | CA | PRO | A | 113 | 93.282 | 28.129 | −9.517 | 1.00 | 28.93 | C |
| ATOM | 623 | CB | PRO | A | 113 | 94.697 | 28.557 | −9.102 | 1.00 | 29.22 | C |
| ATOM | 624 | CG | PRO | A | 113 | 94.817 | 29.977 | −9.608 | 1.00 | 29.25 | C |
| ATOM | 625 | CD | PRO | A | 113 | 93.444 | 30.560 | −9.383 | 1.00 | 28.81 | C |
| ATOM | 626 | C | PRO | A | 113 | 92.642 | 27.163 | −8.505 | 1.00 | 28.63 | C |
| ATOM | 627 | O | PRO | A | 113 | 92.473 | 25.982 | −8.829 | 1.00 | 28.81 | O |
| ATOM | 628 | N | ASP | A | 114 | 92.239 | 27.664 | −7.340 | 1.00 | 28.09 | N |
| ATOM | 629 | CA | ASP | A | 114 | 91.740 | 26.800 | −6.269 | 1.00 | 27.57 | C |
| ATOM | 630 | CB | ASP | A | 114 | 92.605 | 26.991 | −5.020 | 1.00 | 28.11 | C |
| ATOM | 631 | CG | ASP | A | 114 | 94.078 | 26.644 | −5.272 | 1.00 | 30.45 | C |
| ATOM | 632 | OD1 | ASP | A | 114 | 94.959 | 27.360 | −4.740 | 1.00 | 31.83 | O |
| ATOM | 633 | OD2 | ASP | A | 114 | 94.438 | 25.680 | −5.998 | 1.00 | 30.94 | O |
| ATOM | 634 | C | ASP | A | 114 | 90.252 | 26.962 | −5.921 | 1.00 | 26.62 | C |
| ATOM | 635 | O | ASP | A | 114 | 89.754 | 26.323 | −4.980 | 1.00 | 26.49 | O |
| ATOM | 636 | N | SER | A | 115 | 89.549 | 27.806 | −6.677 | 1.00 | 25.25 | N |
| ATOM | 637 | CA | SER | A | 115 | 88.150 | 28.130 | −6.382 | 1.00 | 23.81 | C |
| ATOM | 638 | CB | SER | A | 115 | 88.100 | 29.182 | −5.276 | 1.00 | 23.83 | C |
| ATOM | 639 | OG | SER | A | 115 | 88.650 | 30.403 | −5.733 | 1.00 | 22.52 | O |
| ATOM | 640 | C | SER | A | 115 | 87.352 | 28.653 | −7.586 | 1.00 | 23.20 | C |
| ATOM | 641 | O | SER | A | 115 | 87.917 | 28.964 | −8.639 | 1.00 | 22.61 | O |
| ATOM | 642 | N | PHE | A | 116 | 86.039 | 28.761 | −7.400 | 1.00 | 22.13 | N |
| ATOM | 643 | CA | PHE | A | 116 | 85.175 | 29.515 | −8.306 | 1.00 | 21.81 | C |
| ATOM | 644 | CB | PHE | A | 116 | 84.074 | 28.627 | −8.901 | 1.00 | 21.79 | C |
| ATOM | 645 | CG | PHE | A | 116 | 84.578 | 27.655 | −9.921 | 1.00 | 22.56 | C |
| ATOM | 646 | CD1 | PHE | A | 116 | 85.096 | 26.425 | −9.532 | 1.00 | 23.50 | C |
| ATOM | 647 | CE1 | PHE | A | 116 | 85.581 | 25.515 | −10.487 | 1.00 | 25.19 | C |
| ATOM | 648 | CZ | PHE | A | 116 | 85.550 | 25.846 | −11.835 | 1.00 | 24.86 | C |
| ATOM | 649 | CE2 | PHE | A | 116 | 85.032 | 27.080 | −12.230 | 1.00 | 25.05 | C |
| ATOM | 650 | CD2 | PHE | A | 116 | 84.551 | 27.974 | −11.271 | 1.00 | 24.09 | C |
| ATOM | 651 | C | PHE | A | 116 | 84.556 | 30.678 | −7.553 | 1.00 | 21.29 | C |
| ATOM | 652 | O | PHE | A | 116 | 84.349 | 30.605 | −6.341 | 1.00 | 21.62 | O |
| ATOM | 653 | N | VAL | A | 117 | 84.280 | 31.757 | −8.275 | 1.00 | 20.75 | N |
| ATOM | 654 | CA | VAL | A | 117 | 83.674 | 32.944 | −7.707 | 1.00 | 19.94 | C |
| ATOM | 655 | CB | VAL | A | 117 | 84.628 | 34.134 | −7.791 | 1.00 | 20.10 | C |
| ATOM | 656 | CG1 | VAL | A | 117 | 84.036 | 35.341 | −7.089 | 1.00 | 19.06 | C |
| ATOM | 657 | CG2 | VAL | A | 117 | 86.019 | 33.768 | −7.189 | 1.00 | 20.50 | C |
| ATOM | 658 | C | VAL | A | 117 | 82.399 | 33.242 | −8.489 | 1.00 | 19.85 | C |
| ATOM | 659 | O | VAL | A | 117 | 82.441 | 33.393 | −9.713 | 1.00 | 20.09 | O |
| ATOM | 660 | N | LEU | A | 118 | 81.276 | 33.327 | −7.785 | 1.00 | 19.32 | N |
| ATOM | 661 | CA | LEU | A | 118 | 79.981 | 33.584 | −8.400 | 1.00 | 19.32 | C |
| ATOM | 662 | CB | LEU | A | 118 | 78.936 | 32.565 | −7.912 | 1.00 | 19.30 | C |
| ATOM | 663 | CG | LEU | A | 118 | 78.914 | 31.157 | −8.510 | 1.00 | 20.91 | C |
| ATOM | 664 | CD1 | LEU | A | 118 | 80.203 | 30.381 | −8.224 | 1.00 | 23.74 | C |
| ATOM | 665 | CD2 | LEU | A | 118 | 77.741 | 30.382 | −7.949 | 1.00 | 22.22 | C |
| ATOM | 666 | C | LEU | A | 118 | 79.514 | 34.981 | −8.051 | 1.00 | 19.31 | C |
| ATOM | 667 | O | LEU | A | 118 | 79.574 | 35.387 | −6.887 | 1.00 | 19.13 | O |
| ATOM | 668 | N | ILE | A | 119 | 79.048 | 35.715 | −9.062 | 1.00 | 19.23 | N |
| ATOM | 669 | CA | ILE | A | 119 | 78.521 | 37.053 | −8.860 | 1.00 | 19.06 | C |
| ATOM | 670 | CB | ILE | A | 119 | 79.093 | 38.062 | −9.898 | 1.00 | 19.52 | C |
| ATOM | 671 | CG1 | ILE | A | 119 | 80.627 | 37.931 | −10.022 | 1.00 | 19.59 | C |
| ATOM | 672 | CD1 | ILE | A | 119 | 81.434 | 38.222 | −8.736 | 1.00 | 19.03 | C |
| ATOM | 673 | CG2 | ILE | A | 119 | 78.652 | 39.509 | −9.557 | 1.00 | 18.88 | C |
| ATOM | 674 | C | ILE | A | 119 | 77.008 | 36.958 | −8.938 | 1.00 | 19.43 | C |
| ATOM | 675 | O | ILE | A | 119 | 76.446 | 36.592 | −9.977 | 1.00 | 19.01 | O |
| ATOM | 676 | N | LEU | A | 120 | 76.358 | 37.266 | −7.823 | 1.00 | 19.78 | N |
| ATOM | 677 | CA | LEU | A | 120 | 74.913 | 37.097 | −7.683 | 1.00 | 20.66 | C |
| ATOM | 678 | CB | LEU | A | 120 | 74.609 | 36.193 | −6.483 | 1.00 | 20.51 | C |
| ATOM | 679 | CG | LEU | A | 120 | 75.197 | 34.788 | −6.594 | 1.00 | 21.71 | C |
| ATOM | 680 | CD1 | LEU | A | 120 | 75.218 | 34.103 | −5.236 | 1.00 | 23.12 | C |
| ATOM | 681 | CD2 | LEU | A | 120 | 74.403 | 33.967 | −7.591 | 1.00 | 22.78 | C |
| ATOM | 682 | C | LEU | A | 120 | 74.253 | 38.436 | −7.455 | 1.00 | 20.91 | C |
| ATOM | 683 | O | LEU | A | 120 | 74.853 | 39.319 | −6.848 | 1.00 | 20.71 | O |
| ATOM | 684 | N | GLU | A | 121 | 73.015 | 38.588 | −7.919 | 1.00 | 21.58 | N |
| ATOM | 685 | CA | GLU | A | 121 | 72.238 | 39.775 | −7.581 | 1.00 | 22.82 | C |
| ATOM | 686 | CB | GLU | A | 121 | 70.883 | 39.780 | −8.308 | 1.00 | 23.93 | C |
| ATOM | 687 | CG | GLU | A | 121 | 69.759 | 39.096 | −7.559 | 1.00 | 26.70 | C |
| ATOM | 688 | CD | GLU | A | 121 | 68.493 | 38.950 | −8.387 | 1.00 | 30.59 | C |
| ATOM | 689 | OE1 | GLU | A | 121 | 67.830 | 39.973 | −8.654 | 1.00 | 33.17 | O |
| ATOM | 690 | OE2 | GLU | A | 121 | 68.159 | 37.811 | −8.759 | 1.00 | 31.26 | O |
| ATOM | 691 | C | GLU | A | 121 | 72.062 | 39.836 | −6.065 | 1.00 | 22.52 | C |
| ATOM | 692 | O | GLU | A | 121 | 72.087 | 38.800 | −5.391 | 1.00 | 22.12 | O |
| ATOM | 693 | N | ARG | A | 122 | 71.908 | 41.043 | −5.533 | 1.00 | 22.16 | N |
| ATOM | 694 | CA | ARG | A | 122 | 71.677 | 41.212 | −4.105 | 1.00 | 22.66 | C |
| ATOM | 695 | CB | ARG | A | 122 | 72.977 | 41.620 | −3.390 | 1.00 | 21.96 | C |
| ATOM | 696 | CG | ARG | A | 122 | 72.814 | 41.952 | −1.920 | 1.00 | 21.41 | C |
| ATOM | 697 | CD | ARG | A | 122 | 74.128 | 42.195 | −1.161 | 1.00 | 21.79 | C |
| ATOM | 698 | NE | ARG | A | 122 | 74.932 | 43.293 | −1.726 | 1.00 | 20.54 | N |
| ATOM | 699 | CZ | ARG | A | 122 | 74.781 | 44.581 | −1.418 | 1.00 | 21.80 | C |
| ATOM | 700 | NH1 | ARG | A | 122 | 73.860 | 44.973 | −0.543 | 1.00 | 21.45 | N |
| ATOM | 701 | NH2 | ARG | A | 122 | 75.568 | 45.489 | −1.977 | 1.00 | 23.39 | N |
| ATOM | 702 | C | ARG | A | 122 | 70.575 | 42.249 | −3.864 | 1.00 | 23.50 | C |
| ATOM | 703 | O | ARG | A | 122 | 70.764 | 43.419 | −4.156 | 1.00 | 23.59 | O |
| ATOM | 704 | N | PRO | A | 123 | 69.429 | 41.818 | −3.330 | 1.00 | 24.57 | N |
| ATOM | 705 | CA | PRO | A | 123 | 68.384 | 42.756 | −2.888 | 1.00 | 25.12 | C |
| ATOM | 706 | CB | PRO | A | 123 | 67.233 | 41.832 | −2.448 | 1.00 | 25.10 | C |
| ATOM | 707 | CG | PRO | A | 123 | 67.552 | 40.494 | −3.044 | 1.00 | 25.14 | C |
| ATOM | 708 | CD | PRO | A | 123 | 69.047 | 40.411 | −3.109 | 1.00 | 24.18 | C |
| ATOM | 709 | C | PRO | A | 123 | 68.860 | 43.599 | −1.703 | 1.00 | 25.82 | C |
| ATOM | 710 | O | PRO | A | 123 | 69.678 | 43.129 | −0.900 | 1.00 | 25.56 | O |
| ATOM | 711 | N | GLU | A | 124 | 68.358 | 44.830 | −1.607 | 1.00 | 26.56 | N |
| ATOM | 712 | CA | GLU | A | 124 | 68.738 | 45.750 | −0.533 | 1.00 | 27.26 | C |
| ATOM | 713 | CB | GLU | A | 124 | 69.952 | 46.591 | −0.958 | 1.00 | 27.95 | C |
| ATOM | 714 | CG | GLU | A | 124 | 70.730 | 47.274 | 0.171 | 1.00 | 30.91 | C |
| ATOM | 715 | CD | GLU | A | 124 | 71.867 | 48.140 | −0.367 | 1.00 | 35.36 | C |
| ATOM | 716 | OE1 | GLU | A | 124 | 71.616 | 48.930 | −1.311 | 1.00 | 37.56 | O |
| ATOM | 717 | OE2 | GLU | A | 124 | 73.017 | 48.036 | 0.134 | 1.00 | 36.68 | O |
| ATOM | 718 | C | GLU | A | 124 | 67.544 | 46.649 | −0.201 | 1.00 | 26.82 | C |
| ATOM | 719 | O | GLU | A | 124 | 67.053 | 47.358 | −1.078 | 1.00 | 27.29 | O |
| ATOM | 720 | N | PRO | A | 125 | 67.061 | 46.617 | 1.045 | 1.00 | 25.92 | N |
| ATOM | 721 | CA | PRO | A | 125 | 67.599 | 45.755 | 2.101 | 1.00 | 24.79 | C |
| ATOM | 722 | CB | PRO | A | 125 | 67.062 | 46.403 | 3.373 | 1.00 | 24.95 | C |
| ATOM | 723 | CG | PRO | A | 125 | 65.759 | 46.993 | 2.960 | 1.00 | 24.96 | C |
| ATOM | 724 | CD | PRO | A | 125 | 65.936 | 47.437 | 1.530 | 1.00 | 25.81 | C |
| ATOM | 725 | C | PRO | A | 125 | 67.095 | 44.316 | 1.989 | 1.00 | 23.97 | C |
| ATOM | 726 | O | PRO | A | 125 | 66.109 | 44.040 | 1.287 | 1.00 | 23.35 | O |
| ATOM | 727 | N | VAL | A | 126 | 67.789 | 43.412 | 2.670 | 1.00 | 23.02 | N |
| ATOM | 728 | CA | VAL | A | 126 | 67.507 | 41.987 | 2.583 | 1.00 | 22.32 | C |
| ATOM | 729 | CB | VAL | A | 126 | 68.333 | 41.305 | 1.439 | 1.00 | 22.37 | C |
| ATOM | 730 | CG1 | VAL | A | 126 | 69.815 | 41.129 | 1.837 | 1.00 | 21.96 | C |
| ATOM | 731 | CG2 | VAL | A | 126 | 67.732 | 39.971 | 1.028 | 1.00 | 22.11 | C |
| ATOM | 732 | C | VAL | A | 126 | 67.809 | 41.342 | 3.925 | 1.00 | 22.20 | C |
| ATOM | 733 | O | VAL | A | 126 | 68.600 | 41.866 | 4.723 | 1.00 | 22.19 | O |
| ATOM | 734 | N | GLN | A | 127 | 67.159 | 40.209 | 4.166 | 1.00 | 21.35 | N |
| ATOM | 735 | CA | GLN | A | 127 | 67.429 | 39.378 | 5.323 | 1.00 | 20.84 | C |
| ATOM | 736 | CB | GLN | A | 127 | 66.653 | 39.883 | 6.540 | 1.00 | 20.45 | C |
| ATOM | 737 | CG | GLN | A | 127 | 66.866 | 39.053 | 7.796 | 1.00 | 20.49 | C |
| ATOM | 738 | CD | GLN | A | 127 | 66.156 | 39.632 | 8.999 | 1.00 | 22.00 | C |
| ATOM | 739 | OE1 | GLN | A | 127 | 64.953 | 39.891 | 8.944 | 1.00 | 22.10 | O |
| ATOM | 740 | NE2 | GLN | A | 127 | 66.892 | 39.842 | 10.082 | 1.00 | 20.41 | N |
| ATOM | 741 | C | GLN | A | 127 | 66.996 | 37.952 | 4.963 | 1.00 | 20.56 | C |
| ATOM | 742 | O | GLN | A | 127 | 65.917 | 37.760 | 4.392 | 1.00 | 20.00 | O |
| ATOM | 743 | N | ASP | A | 128 | 67.845 | 36.967 | 5.250 | 1.00 | 19.63 | N |
| ATOM | 744 | CA | ASP | A | 128 | 67.454 | 35.593 | 5.008 | 1.00 | 19.54 | C |
| ATOM | 745 | CB | ASP | A | 128 | 68.672 | 34.650 | 4.844 | 1.00 | 19.72 | C |
| ATOM | 746 | CG | ASP | A | 128 | 69.276 | 34.181 | 6.158 | 1.00 | 21.27 | C |
| ATOM | 747 | OD1 | ASP | A | 128 | 68.578 | 34.093 | 7.189 | 1.00 | 22.90 | O |
| ATOM | 748 | OD2 | ASP | A | 128 | 70.480 | 33.843 | 6.237 | 1.00 | 24.10 | O |
| ATOM | 749 | C | ASP | A | 128 | 66.381 | 35.126 | 6.016 | 1.00 | 19.14 | C |
| ATOM | 750 | O | ASP | A | 128 | 66.220 | 35.724 | 7.079 | 1.00 | 18.98 | O |
| ATOM | 751 | N | LEU | A | 129 | 65.642 | 34.077 | 5.658 | 1.00 | 18.65 | N |
| ATOM | 752 | CA | LEU | A | 129 | 64.485 | 33.651 | 6.429 | 1.00 | 18.28 | C |
| ATOM | 753 | CB | LEU | A | 129 | 63.611 | 32.662 | 5.619 | 1.00 | 17.80 | C |
| ATOM | 754 | CG | LEU | A | 129 | 62.291 | 32.181 | 6.245 | 1.00 | 17.80 | C |
| ATOM | 755 | CD1 | LEU | A | 129 | 61.344 | 33.350 | 6.565 | 1.00 | 16.86 | C |
| ATOM | 756 | CD2 | LEU | A | 129 | 61.591 | 31.180 | 5.327 | 1.00 | 15.45 | C |
| ATOM | 757 | C | LEU | A | 129 | 64.861 | 33.096 | 7.804 | 1.00 | 18.57 | C |
| ATOM | 758 | O | LEU | A | 129 | 64.095 | 33.220 | 8.760 | 1.00 | 18.46 | O |
| ATOM | 759 | N | PHE | A | 130 | 66.047 | 32.503 | 7.908 | 1.00 | 18.90 | N |
| ATOM | 760 | CA | PHE | A | 130 | 66.545 | 32.032 | 9.200 | 1.00 | 19.18 | C |
| ATOM | 761 | CB | PHE | A | 130 | 67.887 | 31.311 | 9.033 | 1.00 | 19.86 | C |
| ATOM | 762 | CG | PHE | A | 130 | 68.531 | 30.931 | 10.339 | 1.00 | 22.10 | C |
| ATOM | 763 | CD1 | PHE | A | 130 | 69.471 | 31.764 | 10.933 | 1.00 | 23.65 | C |
| ATOM | 764 | CE1 | PHE | A | 130 | 70.069 | 31.423 | 12.155 | 1.00 | 26.57 | C |
| ATOM | 765 | CZ | PHE | A | 130 | 69.712 | 30.232 | 12.792 | 1.00 | 25.84 | C |
| ATOM | 766 | CE2 | PHE | A | 130 | 68.765 | 29.398 | 12.206 | 1.00 | 26.84 | C |
| ATOM | 767 | CD2 | PHE | A | 130 | 68.179 | 29.748 | 10.982 | 1.00 | 24.38 | C |
| ATOM | 768 | C | PHE | A | 130 | 66.704 | 33.176 | 10.203 | 1.00 | 19.08 | C |
| ATOM | 769 | O | PHE | A | 130 | 66.287 | 33.060 | 11.374 | 1.00 | 17.75 | O |
| ATOM | 770 | N | ASP | A | 131 | 67.316 | 34.274 | 9.753 | 1.00 | 19.37 | N |
| ATOM | 771 | CA | ASP | A | 131 | 67.489 | 35.442 | 10.622 | 1.00 | 20.03 | C |
| ATOM | 772 | CB | ASP | A | 131 | 68.375 | 36.505 | 9.966 | 1.00 | 20.64 | C |
| ATOM | 773 | CG | ASP | A | 131 | 69.836 | 36.090 | 9.894 | 1.00 | 23.72 | C |
| ATOM | 774 | OD1 | ASP | A | 131 | 70.258 | 35.197 | 10.671 | 1.00 | 28.11 | O |
| ATOM | 775 | OD2 | ASP | A | 131 | 70.642 | 36.603 | 9.084 | 1.00 | 27.01 | O |
| ATOM | 776 | C | ASP | A | 131 | 66.136 | 36.030 | 10.947 | 1.00 | 19.62 | C |
| ATOM | 777 | O | ASP | A | 131 | 65.868 | 36.368 | 12.086 | 1.00 | 19.32 | O |
| ATOM | 778 | N | PHE | A | 132 | 65.275 | 36.133 | 9.936 | 1.00 | 19.50 | N |
| ATOM | 779 | CA | PHE | A | 132 | 63.969 | 36.758 | 10.094 | 1.00 | 19.48 | C |
| ATOM | 780 | CB | PHE | A | 132 | 63.233 | 36.740 | 8.754 | 1.00 | 19.50 | C |
| ATOM | 781 | CG | PHE | A | 132 | 61.939 | 37.481 | 8.749 | 1.00 | 20.08 | C |
| ATOM | 782 | CD1 | PHE | A | 132 | 61.906 | 38.839 | 8.455 | 1.00 | 21.55 | C |
| ATOM | 783 | CE1 | PHE | A | 132 | 60.704 | 39.535 | 8.433 | 1.00 | 22.42 | C |
| ATOM | 784 | CZ | PHE | A | 132 | 59.506 | 38.861 | 8.680 | 1.00 | 22.64 | C |
| ATOM | 785 | CE2 | PHE | A | 132 | 59.522 | 37.505 | 8.962 | 1.00 | 21.42 | C |
| ATOM | 786 | CD2 | PHE | A | 132 | 60.734 | 36.814 | 8.991 | 1.00 | 21.38 | C |
| ATOM | 787 | C | PHE | A | 132 | 63.186 | 36.015 | 11.167 | 1.00 | 20.30 | C |
| ATOM | 788 | O | PHE | A | 132 | 62.643 | 36.642 | 12.088 | 1.00 | 20.08 | O |
| ATOM | 789 | N | ILE | A | 133 | 63.131 | 34.682 | 11.064 | 1.00 | 20.79 | N |
| ATOM | 790 | CA | ILE | A | 133 | 62.411 | 33.875 | 12.064 | 1.00 | 21.52 | C |
| ATOM | 791 | CB | ILE | A | 133 | 62.195 | 32.429 | 11.583 | 1.00 | 21.38 | C |
| ATOM | 792 | CG1 | ILE | A | 133 | 61.215 | 32.402 | 10.402 | 1.00 | 19.84 | C |
| ATOM | 793 | CD1 | ILE | A | 133 | 61.200 | 31.104 | 9.665 | 1.00 | 16.74 | C |
| ATOM | 794 | CG2 | ILE | A | 133 | 61.665 | 31.539 | 12.747 | 1.00 | 21.31 | C |
| ATOM | 795 | C | ILE | A | 133 | 63.097 | 33.868 | 13.438 | 1.00 | 22.84 | C |
| ATOM | 796 | O | ILE | A | 133 | 62.430 | 33.825 | 14.472 | 1.00 | 22.75 | O |
| ATOM | 797 | N | THR | A | 134 | 64.423 | 33.888 | 13.446 | 1.00 | 23.77 | N |
| ATOM | 798 | CA | THR | A | 134 | 65.167 | 34.000 | 14.696 | 1.00 | 25.24 | C |
| ATOM | 799 | CB | THR | A | 134 | 66.683 | 33.972 | 14.427 | 1.00 | 24.97 | C |
| ATOM | 800 | OG1 | THR | A | 134 | 67.056 | 32.682 | 13.921 | 1.00 | 24.99 | O |
| ATOM | 801 | CG2 | THR | A | 134 | 67.486 | 34.101 | 15.735 | 1.00 | 25.41 | C |
| ATOM | 802 | C | THR | A | 134 | 64.779 | 35.286 | 15.433 | 1.00 | 26.05 | C |
| ATOM | 803 | O | THR | A | 134 | 64.514 | 35.271 | 16.636 | 1.00 | 26.27 | O |
| ATOM | 804 | N | GLU | A | 135 | 64.728 | 36.386 | 14.693 | 1.00 | 27.17 | N |
| ATOM | 805 | CA | GLU | A | 135 | 64.424 | 37.693 | 15.268 | 1.00 | 28.48 | C |
| ATOM | 806 | CB | GLU | A | 135 | 64.830 | 38.807 | 14.302 | 1.00 | 28.91 | C |
| ATOM | 807 | CG | GLU | A | 135 | 66.282 | 39.221 | 14.449 | 1.00 | 32.87 | C |
| ATOM | 808 | CD | GLU | A | 135 | 66.702 | 40.288 | 13.450 | 1.00 | 37.37 | C |
| ATOM | 809 | OE1 | GLU | A | 135 | 65.813 | 41.022 | 12.939 | 1.00 | 38.58 | O |
| ATOM | 810 | OE2 | GLU | A | 135 | 67.927 | 40.383 | 13.177 | 1.00 | 38.32 | O |
| ATOM | 811 | C | GLU | A | 135 | 62.958 | 37.853 | 15.657 | 1.00 | 28.36 | C |
| ATOM | 812 | O | GLU | A | 135 | 62.656 | 38.416 | 16.710 | 1.00 | 27.93 | O |
| ATOM | 813 | N | ARG | A | 136 | 62.056 | 37.345 | 14.817 | 1.00 | 27.83 | N |
| ATOM | 814 | CA | ARG | A | 136 | 60.635 | 37.631 | 14.983 | 1.00 | 27.91 | C |
| ATOM | 815 | CB | ARG | A | 136 | 60.022 | 38.109 | 13.657 | 1.00 | 28.19 | C |
| ATOM | 816 | CG | ARG | A | 136 | 60.551 | 39.487 | 13.244 | 1.00 | 30.84 | C |
| ATOM | 817 | CD | ARG | A | 136 | 60.046 | 40.034 | 11.909 | 1.00 | 33.40 | C |
| ATOM | 818 | NE | ARG | A | 136 | 58.583 | 40.081 | 11.805 | 1.00 | 35.56 | N |
| ATOM | 819 | CZ | ARG | A | 136 | 57.907 | 40.978 | 11.081 | 1.00 | 35.76 | C |
| ATOM | 820 | NH1 | ARG | A | 136 | 58.556 | 41.923 | 10.403 | 1.00 | 35.79 | N |
| ATOM | 821 | NH2 | ARG | A | 136 | 56.580 | 40.938 | 11.041 | 1.00 | 34.21 | N |
| ATOM | 822 | C | ARG | A | 136 | 59.836 | 36.488 | 15.594 | 1.00 | 26.86 | C |
| ATOM | 823 | O | ARG | A | 136 | 58.683 | 36.677 | 15.980 | 1.00 | 27.36 | O |
| ATOM | 824 | N | GLY | A | 137 | 60.452 | 35.316 | 15.713 | 1.00 | 25.61 | N |
| ATOM | 825 | CA | GLY | A | 137 | 59.754 | 34.134 | 16.187 | 1.00 | 24.72 | C |
| ATOM | 826 | C | GLY | A | 137 | 58.763 | 33.584 | 15.156 | 1.00 | 24.39 | C |
| ATOM | 827 | O | GLY | A | 137 | 58.796 | 33.952 | 13.969 | 1.00 | 23.90 | O |
| ATOM | 828 | N | ALA | A | 138 | 57.880 | 32.699 | 15.615 | 1.00 | 23.04 | N |
| ATOM | 829 | CA | ALA | A | 138 | 56.864 | 32.089 | 14.760 | 1.00 | 22.25 | C |
| ATOM | 830 | CB | ALA | A | 138 | 55.895 | 31.269 | 15.612 | 1.00 | 22.29 | C |
| ATOM | 831 | C | ALA | A | 138 | 56.101 | 33.152 | 13.968 | 1.00 | 22.02 | C |
| ATOM | 832 | O | ALA | A | 138 | 55.694 | 34.174 | 14.523 | 1.00 | 21.11 | O |
| ATOM | 833 | N | LEU | A | 139 | 55.914 | 32.906 | 12.671 | 1.00 | 20.97 | N |
| ATOM | 834 | CA | LEU | A | 139 | 55.223 | 33.860 | 11.814 | 1.00 | 20.45 | C |
| ATOM | 835 | CB | LEU | A | 139 | 55.673 | 33.676 | 10.358 | 1.00 | 19.75 | C |
| ATOM | 836 | CG | LEU | A | 139 | 57.194 | 33.668 | 10.121 | 1.00 | 19.78 | C |
| ATOM | 837 | CD1 | LEU | A | 139 | 57.509 | 33.578 | 8.624 | 1.00 | 17.80 | C |
| ATOM | 838 | CD2 | LEU | A | 139 | 57.871 | 34.908 | 10.772 | 1.00 | 19.37 | C |
| ATOM | 839 | C | LEU | A | 139 | 53.706 | 33.707 | 11.938 | 1.00 | 20.32 | C |
| ATOM | 840 | O | LEU | A | 139 | 53.209 | 32.589 | 12.007 | 1.00 | 20.36 | O |
| ATOM | 841 | N | GLN | A | 140 | 52.979 | 34.828 | 11.950 | 1.00 | 19.81 | N |
| ATOM | 842 | CA | GLN | A | 140 | 51.530 | 34.796 | 11.751 | 1.00 | 19.77 | C |
| ATOM | 843 | CB | GLN | A | 140 | 50.958 | 36.210 | 11.624 | 1.00 | 20.35 | C |
| ATOM | 844 | CG | GLN | A | 140 | 50.938 | 37.006 | 12.913 | 1.00 | 24.46 | C |
| ATOM | 845 | CD | GLN | A | 140 | 50.666 | 38.484 | 12.674 | 1.00 | 30.48 | C |
| ATOM | 846 | OE1 | GLN | A | 140 | 49.836 | 38.846 | 11.827 | 1.00 | 32.68 | O |
| ATOM | 847 | NE2 | GLN | A | 140 | 51.357 | 39.343 | 13.421 | 1.00 | 32.67 | N |
| ATOM | 848 | C | GLN | A | 140 | 51.211 | 34.035 | 10.469 | 1.00 | 18.78 | C |
| ATOM | 849 | O | GLN | A | 140 | 51.957 | 34.118 | 9.494 | 1.00 | 17.03 | O |
| ATOM | 850 | N | GLU | A | 141 | 50.088 | 33.325 | 10.453 | 1.00 | 18.57 | N |
| ATOM | 851 | CA | GLU | A | 141 | 49.769 | 32.482 | 9.296 | 1.00 | 18.81 | C |
| ATOM | 852 | CB | GLU | A | 141 | 48.563 | 31.588 | 9.579 | 1.00 | 18.86 | C |
| ATOM | 853 | CG | GLU | A | 141 | 48.922 | 30.461 | 10.531 | 1.00 | 20.50 | C |
| ATOM | 854 | CD | GLU | A | 141 | 47.785 | 29.504 | 10.762 | 1.00 | 20.11 | C |
| ATOM | 855 | OE1 | GLU | A | 141 | 47.107 | 29.151 | 9.779 | 1.00 | 20.79 | O |
| ATOM | 856 | OE2 | GLU | A | 141 | 47.572 | 29.120 | 11.933 | 1.00 | 21.96 | O |
| ATOM | 857 | C | GLU | A | 141 | 49.605 | 33.235 | 7.970 | 1.00 | 18.75 | C |
| ATOM | 858 | O | GLU | A | 141 | 49.969 | 32.702 | 6.931 | 1.00 | 18.07 | O |
| ATOM | 859 | N | GLU | A | 142 | 49.048 | 34.454 | 8.002 | 1.00 | 18.37 | N |
| ATOM | 860 | CA | GLU | A | 142 | 48.939 | 35.263 | 6.787 | 1.00 | 18.46 | C |
| ATOM | 861 | CB | GLU | A | 142 | 48.216 | 36.589 | 7.078 | 1.00 | 18.72 | C |
| ATOM | 862 | CG | GLU | A | 142 | 48.076 | 37.515 | 5.889 | 1.00 | 20.48 | C |
| ATOM | 863 | CD | GLU | A | 142 | 47.411 | 38.836 | 6.241 | 1.00 | 24.11 | C |
| ATOM | 864 | OE1 | GLU | A | 142 | 48.061 | 39.692 | 6.891 | 1.00 | 23.55 | O |
| ATOM | 865 | OE2 | GLU | A | 142 | 46.232 | 39.017 | 5.851 | 1.00 | 25.93 | O |
| ATOM | 866 | C | GLU | A | 142 | 50.329 | 35.529 | 6.179 | 1.00 | 17.83 | C |
| ATOM | 867 | O | GLU | A | 142 | 50.506 | 35.530 | 4.959 | 1.00 | 17.80 | O |
| ATOM | 868 | N | LEU | A | 143 | 51.309 | 35.761 | 7.037 | 1.00 | 16.88 | N |
| ATOM | 869 | CA | LEU | A | 143 | 52.653 | 36.029 | 6.568 | 1.00 | 16.41 | C |
| ATOM | 870 | CB | LEU | A | 143 | 53.497 | 36.667 | 7.666 | 1.00 | 16.81 | C |
| ATOM | 871 | CG | LEU | A | 143 | 54.952 | 36.998 | 7.288 | 1.00 | 16.83 | C |
| ATOM | 872 | CD1 | LEU | A | 143 | 54.999 | 37.909 | 6.049 | 1.00 | 15.94 | C |
| ATOM | 873 | CD2 | LEU | A | 143 | 55.625 | 37.670 | 8.464 | 1.00 | 16.70 | C |
| ATOM | 874 | C | LEU | A | 143 | 53.307 | 34.749 | 6.057 | 1.00 | 15.72 | C |
| ATOM | 875 | O | LEU | A | 143 | 53.921 | 34.745 | 4.983 | 1.00 | 15.44 | O |
| ATOM | 876 | N | ALA | A | 144 | 53.173 | 33.670 | 6.824 | 1.00 | 14.81 | N |
| ATOM | 877 | CA | ALA | A | 144 | 53.692 | 32.364 | 6.404 | 1.00 | 14.61 | C |
| ATOM | 878 | CB | ALA | A | 144 | 53.444 | 31.327 | 7.470 | 1.00 | 14.33 | C |
| ATOM | 879 | C | ALA | A | 144 | 53.078 | 31.914 | 5.077 | 1.00 | 14.82 | C |
| ATOM | 880 | O | ALA | A | 144 | 53.754 | 31.270 | 4.253 | 1.00 | 14.19 | O |
| ATOM | 881 | N | ARG | A | 145 | 51.796 | 32.235 | 4.884 | 1.00 | 14.19 | N |
| ATOM | 882 | CA | ARG | A | 145 | 51.090 | 31.869 | 3.666 | 1.00 | 15.16 | C |
| ATOM | 883 | CB | ARG | A | 145 | 49.587 | 32.205 | 3.764 | 1.00 | 15.23 | C |
| ATOM | 884 | CG | ARG | A | 145 | 48.803 | 32.031 | 2.453 | 1.00 | 16.28 | C |
| ATOM | 885 | CD | ARG | A | 145 | 47.303 | 32.380 | 2.564 | 1.00 | 18.20 | C |
| ATOM | 886 | NE | ARG | A | 145 | 46.693 | 31.573 | 3.615 | 1.00 | 17.28 | N |
| ATOM | 887 | CZ | ARG | A | 145 | 46.238 | 32.049 | 4.761 | 1.00 | 17.72 | C |
| ATOM | 888 | NH1 | ARG | A | 145 | 46.270 | 33.352 | 5.018 | 1.00 | 17.11 | N |
| ATOM | 889 | NH2 | ARG | A | 145 | 45.747 | 31.212 | 5.657 | 1.00 | 18.63 | N |
| ATOM | 890 | C | ARG | A | 145 | 51.727 | 32.562 | 2.471 | 1.00 | 15.43 | C |
| ATOM | 891 | O | ARG | A | 145 | 52.034 | 31.917 | 1.470 | 1.00 | 16.61 | O |
| ATOM | 892 | N | SER | A | 146 | 51.941 | 33.868 | 2.578 | 1.00 | 15.08 | N |
| ATOM | 893 | CA | SER | A | 146 | 52.557 | 34.618 | 1.491 | 1.00 | 15.89 | C |
| ATOM | 894 | CB | SER | A | 146 | 52.558 | 36.114 | 1.823 | 1.00 | 15.77 | C |
| ATOM | 895 | OG | SER | A | 146 | 53.374 | 36.817 | 0.907 | 1.00 | 18.17 | O |
| ATOM | 896 | C | SER | A | 146 | 53.976 | 34.104 | 1.170 | 1.00 | 15.75 | C |
| ATOM | 897 | O | SER | A | 146 | 54.311 | 33.849 | 0.000 | 1.00 | 15.69 | O |
| ATOM | 898 | N | PHE | A | 147 | 54.777 | 33.926 | 2.220 | 1.00 | 15.20 | N |
| ATOM | 899 | CA | PHE | A | 147 | 56.145 | 33.423 | 2.104 | 1.00 | 15.40 | C |
| ATOM | 900 | CB | PHE | A | 147 | 56.801 | 33.392 | 3.487 | 1.00 | 15.25 | C |
| ATOM | 901 | CG | PHE | A | 147 | 57.345 | 34.724 | 3.939 | 1.00 | 16.31 | C |
| ATOM | 902 | CD1 | PHE | A | 147 | 57.041 | 35.903 | 3.246 | 1.00 | 17.31 | C |
| ATOM | 903 | CE1 | PHE | A | 147 | 57.552 | 37.121 | 3.663 | 1.00 | 18.81 | C |
| ATOM | 904 | CZ | PHE | A | 147 | 58.389 | 37.178 | 4.790 | 1.00 | 18.13 | C |
| ATOM | 905 | CE2 | PHE | A | 147 | 58.696 | 36.017 | 5.480 | 1.00 | 17.81 | C |
| ATOM | 906 | CD2 | PHE | A | 147 | 58.176 | 34.793 | 5.052 | 1.00 | 16.34 | C |
| ATOM | 907 | C | PHE | A | 147 | 56.195 | 32.024 | 1.483 | 1.00 | 14.94 | C |
| ATOM | 908 | O | PHE | A | 147 | 56.927 | 31.786 | 0.522 | 1.00 | 15.80 | O |
| ATOM | 909 | N | PHE | A | 148 | 55.407 | 31.114 | 2.033 | 1.00 | 14.80 | N |
| ATOM | 910 | CA | PHE | A | 148 | 55.354 | 29.733 | 1.549 | 1.00 | 15.37 | C |
| ATOM | 911 | CB | PHE | A | 148 | 54.409 | 28.887 | 2.418 | 1.00 | 14.71 | C |
| ATOM | 912 | CG | PHE | A | 148 | 54.574 | 27.399 | 2.224 | 1.00 | 14.42 | C |
| ATOM | 913 | CD1 | PHE | A | 148 | 55.810 | 26.776 | 2.456 | 1.00 | 13.47 | C |
| ATOM | 914 | CE1 | PHE | A | 148 | 55.962 | 25.379 | 2.277 | 1.00 | 10.13 | C |
| ATOM | 915 | CZ | PHE | A | 148 | 54.876 | 24.618 | 1.864 | 1.00 | 12.94 | C |
| ATOM | 916 | CE2 | PHE | A | 148 | 53.635 | 25.237 | 1.625 | 1.00 | 14.02 | C |
| ATOM | 917 | CD2 | PHE | A | 148 | 53.495 | 26.622 | 1.813 | 1.00 | 14.27 | C |
| ATOM | 918 | C | PHE | A | 148 | 54.898 | 29.648 | 0.089 | 1.00 | 15.20 | C |
| ATOM | 919 | O | PHE | A | 148 | 55.459 | 28.902 | −0.703 | 1.00 | 14.97 | O |
| ATOM | 920 | N | TRP | A | 149 | 53.866 | 30.413 | −0.253 | 1.00 | 15.54 | N |
| ATOM | 921 | CA | TRP | A | 149 | 53.393 | 30.477 | −1.634 | 1.00 | 15.37 | C |
| ATOM | 922 | CB | TRP | A | 149 | 52.230 | 31.470 | −1.739 | 1.00 | 15.34 | C |
| ATOM | 923 | CG | TRP | A | 149 | 51.671 | 31.606 | −3.110 | 1.00 | 15.24 | C |
| ATOM | 924 | CD1 | TRP | A | 149 | 52.070 | 32.494 | −4.075 | 1.00 | 14.51 | C |
| ATOM | 925 | NE1 | TRP | A | 149 | 51.301 | 32.333 | −5.205 | 1.00 | 15.75 | N |
| ATOM | 926 | CE2 | TRP | A | 149 | 50.394 | 31.326 | −4.998 | 1.00 | 15.41 | C |
| ATOM | 927 | CD2 | TRP | A | 149 | 50.595 | 30.845 | −3.682 | 1.00 | 15.51 | C |
| ATOM | 928 | CE3 | TRP | A | 149 | 49.766 | 29.804 | −3.210 | 1.00 | 15.20 | C |
| ATOM | 929 | CZ3 | TRP | A | 149 | 48.777 | 29.287 | −4.064 | 1.00 | 14.40 | C |
| ATOM | 930 | CH2 | TRP | A | 149 | 48.614 | 29.788 | −5.376 | 1.00 | 15.19 | C |
| ATOM | 931 | CZ2 | TRP | A | 149 | 49.405 | 30.804 | −5.857 | 1.00 | 15.74 | C |
| ATOM | 932 | C | TRP | A | 149 | 54.516 | 30.881 | −2.585 | 1.00 | 15.47 | C |
| ATOM | 933 | O | TRP | A | 149 | 54.709 | 30.266 | −3.637 | 1.00 | 15.67 | O |
| ATOM | 934 | N | GLN | A | 150 | 55.267 | 31.913 | −2.213 | 1.00 | 16.07 | N |
| ATOM | 935 | CA | GLN | A | 150 | 56.354 | 32.394 | −3.063 | 1.00 | 16.16 | C |
| ATOM | 936 | CB | GLN | A | 150 | 56.926 | 33.704 | −2.522 | 1.00 | 16.54 | C |
| ATOM | 937 | CG | GLN | A | 150 | 56.012 | 34.904 | −2.760 | 1.00 | 17.72 | C |
| ATOM | 938 | CD | GLN | A | 150 | 56.654 | 36.188 | −2.309 | 1.00 | 20.82 | C |
| ATOM | 939 | OE1 | GLN | A | 150 | 57.668 | 36.594 | −2.860 | 1.00 | 20.46 | O |
| ATOM | 940 | NE2 | GLN | A | 150 | 56.078 | 36.825 | −1.291 | 1.00 | 22.67 | N |
| ATOM | 941 | C | GLN | A | 150 | 57.470 | 31.366 | −3.231 | 1.00 | 16.22 | C |
| ATOM | 942 | O | GLN | A | 150 | 58.068 | 31.271 | −4.311 | 1.00 | 16.09 | O |
| ATOM | 943 | N | VAL | A | 151 | 57.747 | 30.613 | −2.165 | 1.00 | 15.69 | N |
| ATOM | 944 | CA | VAL | A | 151 | 58.719 | 29.528 | −2.219 | 1.00 | 15.67 | C |
| ATOM | 945 | CB | VAL | A | 151 | 58.973 | 28.914 | −0.819 | 1.00 | 15.75 | C |
| ATOM | 946 | CG1 | VAL | A | 151 | 59.838 | 27.661 | −0.920 | 1.00 | 15.14 | C |
| ATOM | 947 | CG2 | VAL | A | 151 | 59.648 | 29.950 | 0.087 | 1.00 | 14.26 | C |
| ATOM | 948 | C | VAL | A | 151 | 58.232 | 28.454 | −3.186 | 1.00 | 15.73 | C |
| ATOM | 949 | O | VAL | A | 151 | 58.979 | 28.003 | −4.048 | 1.00 | 15.25 | O |
| ATOM | 950 | N | LEU | A | 152 | 56.967 | 28.065 | −3.046 | 1.00 | 16.29 | N |
| ATOM | 951 | CA | LEU | A | 152 | 56.348 | 27.138 | −3.992 | 1.00 | 17.18 | C |
| ATOM | 952 | CB | LEU | A | 152 | 54.859 | 26.947 | −3.658 | 1.00 | 17.37 | C |
| ATOM | 953 | CG | LEU | A | 152 | 54.467 | 25.690 | −2.874 | 1.00 | 19.91 | C |
| ATOM | 954 | CD1 | LEU | A | 152 | 54.643 | 24.445 | −3.756 | 1.00 | 22.90 | C |
| ATOM | 955 | CD2 | LEU | A | 152 | 55.236 | 25.494 | −1.621 | 1.00 | 23.13 | C |
| ATOM | 956 | C | LEU | A | 152 | 56.512 | 27.572 | −5.453 | 1.00 | 16.62 | C |
| ATOM | 957 | O | LEU | A | 152 | 56.889 | 26.765 | −6.299 | 1.00 | 16.65 | O |
| ATOM | 958 | N | GLU | A | 153 | 56.217 | 28.841 | −5.739 | 1.00 | 16.61 | N |
| ATOM | 959 | CA | GLU | A | 153 | 56.333 | 29.375 | −7.096 | 1.00 | 16.62 | C |
| ATOM | 960 | CB | GLU | A | 153 | 55.832 | 30.827 | −7.180 | 1.00 | 16.56 | C |
| ATOM | 961 | CG | GLU | A | 153 | 54.331 | 30.997 | −6.968 | 1.00 | 17.23 | C |
| ATOM | 962 | CD | GLU | A | 153 | 53.514 | 30.514 | −8.156 | 1.00 | 17.86 | C |
| ATOM | 963 | OE1 | GLU | A | 153 | 53.901 | 30.807 | −9.303 | 1.00 | 20.00 | O |
| ATOM | 964 | OE2 | GLU | A | 153 | 52.487 | 29.843 | −7.945 | 1.00 | 17.52 | O |
| ATOM | 965 | C | GLU | A | 153 | 57.777 | 29.297 | −7.568 | 1.00 | 16.68 | C |
| ATOM | 966 | O | GLU | A | 153 | 58.038 | 28.986 | −8.732 | 1.00 | 16.20 | O |
| ATOM | 967 | N | ALA | A | 154 | 58.712 | 29.559 | −6.656 | 1.00 | 16.55 | N |
| ATOM | 968 | CA | ALA | A | 154 | 60.140 | 29.496 | −6.992 | 1.00 | 16.97 | C |
| ATOM | 969 | CB | ALA | A | 154 | 61.004 | 30.188 | −5.919 | 1.00 | 15.90 | C |
| ATOM | 970 | C | ALA | A | 154 | 60.621 | 28.063 | −7.243 | 1.00 | 16.84 | C |
| ATOM | 971 | O | ALA | A | 154 | 61.345 | 27.818 | −8.207 | 1.00 | 17.76 | O |
| ATOM | 972 | N | VAL | A | 155 | 60.218 | 27.126 | −6.386 | 1.00 | 16.64 | N |
| ATOM | 973 | CA | VAL | A | 155 | 60.584 | 25.724 | −6.564 | 1.00 | 16.78 | C |
| ATOM | 974 | CB | VAL | A | 155 | 60.201 | 24.871 | −5.326 | 1.00 | 17.27 | C |
| ATOM | 975 | CG1 | VAL | A | 155 | 60.395 | 23.386 | −5.589 | 1.00 | 16.81 | C |
| ATOM | 976 | CG2 | VAL | A | 155 | 61.032 | 25.313 | −4.084 | 1.00 | 17.68 | C |
| ATOM | 977 | C | VAL | A | 155 | 59.958 | 25.163 | −7.852 | 1.00 | 16.84 | C |
| ATOM | 978 | O | VAL | A | 155 | 60.621 | 24.445 | −8.603 | 1.00 | 16.56 | O |
| ATOM | 979 | N | ARG | A | 156 | 58.690 | 25.491 | −8.107 | 1.00 | 16.70 | N |
| ATOM | 980 | CA | ARG | A | 156 | 58.051 | 25.086 | −9.374 | 1.00 | 17.03 | C |
| ATOM | 981 | CB | ARG | A | 156 | 56.603 | 25.570 | −9.461 | 1.00 | 16.46 | C |
| ATOM | 982 | CG | ARG | A | 156 | 55.645 | 24.827 | −8.564 | 1.00 | 17.08 | C |
| ATOM | 983 | CD | ARG | A | 156 | 54.201 | 25.302 | −8.681 | 1.00 | 16.07 | C |
| ATOM | 984 | NE | ARG | A | 156 | 53.815 | 25.379 | −10.087 | 1.00 | 15.86 | N |
| ATOM | 985 | CZ | ARG | A | 156 | 52.921 | 26.218 | −10.591 | 1.00 | 16.05 | C |
| ATOM | 986 | NH1 | ARG | A | 156 | 52.280 | 27.071 | −9.805 | 1.00 | 14.03 | N |
| ATOM | 987 | NH2 | ARG | A | 156 | 52.672 | 26.199 | −11.895 | 1.00 | 15.89 | N |
| ATOM | 988 | C | ARG | A | 156 | 58.839 | 25.599 | −10.573 | 1.00 | 17.05 | C |
| ATOM | 989 | O | ARG | A | 156 | 59.071 | 24.864 | −11.529 | 1.00 | 17.34 | O |
| ATOM | 990 | N | HIS | A | 157 | 59.266 | 26.855 | −10.522 | 1.00 | 17.35 | N |
| ATOM | 991 | CA | HIS | A | 157 | 60.090 | 27.397 | −11.594 | 1.00 | 18.31 | C |
| ATOM | 992 | CB | HIS | A | 157 | 60.449 | 28.859 | −11.330 | 1.00 | 18.48 | C |
| ATOM | 993 | CG | HIS | A | 157 | 61.374 | 29.443 | −12.351 | 1.00 | 20.28 | C |
| ATOM | 994 | ND1 | HIS | A | 157 | 62.696 | 29.733 | −12.078 | 1.00 | 23.93 | N |
| ATOM | 995 | CE1 | HIS | A | 157 | 63.262 | 30.241 | −13.158 | 1.00 | 23.06 | C |
| ATOM | 996 | NE2 | HIS | A | 157 | 62.356 | 30.288 | −14.118 | 1.00 | 23.55 | N |
| ATOM | 997 | CD2 | HIS | A | 157 | 61.168 | 29.796 | −13.639 | 1.00 | 20.83 | C |
| ATOM | 998 | C | HIS | A | 157 | 61.361 | 26.559 | −11.806 | 1.00 | 18.61 | C |
| ATOM | 999 | O | HIS | A | 157 | 61.691 | 26.199 | −12.947 | 1.00 | 17.98 | O |
| ATOM | 1000 | N | CYS | A | 158 | 62.064 | 26.247 | −10.715 | 1.00 | 18.74 | N |
| ATOM | 1001 | CA | CYS | A | 158 | 63.259 | 25.405 | −10.800 | 1.00 | 19.66 | C |
| ATOM | 1002 | CB | CYS | A | 158 | 63.837 | 25.146 | −9.413 | 1.00 | 19.79 | C |
| ATOM | 1003 | SG | CYS | A | 158 | 64.537 | 26.620 | −8.683 | 1.00 | 22.60 | S |
| ATOM | 1004 | C | CYS | A | 158 | 62.979 | 24.077 | −11.501 | 1.00 | 19.92 | C |
| ATOM | 1005 | O | CYS | A | 158 | 63.677 | 23.712 | −12.447 | 1.00 | 20.10 | O |
| ATOM | 1006 | N | HIS | A | 159 | 61.955 | 23.365 | −11.032 | 1.00 | 20.09 | N |
| ATOM | 1007 | CA | HIS | A | 159 | 61.580 | 22.085 | −11.612 | 1.00 | 20.50 | C |
| ATOM | 1008 | CB | HIS | A | 159 | 60.484 | 21.410 | −10.782 | 1.00 | 20.39 | C |
| ATOM | 1009 | CG | HIS | A | 159 | 60.934 | 20.995 | −9.414 | 1.00 | 21.38 | C |
| ATOM | 1010 | ND1 | HIS | A | 159 | 60.503 | 19.834 | −8.814 | 1.00 | 22.95 | N |
| ATOM | 1011 | CE1 | HIS | A | 159 | 61.055 | 19.727 | −7.616 | 1.00 | 22.06 | C |
| ATOM | 1012 | NE2 | HIS | A | 159 | 61.845 | 20.769 | −7.426 | 1.00 | 21.41 | N |
| ATOM | 1013 | CD2 | HIS | A | 159 | 61.790 | 21.577 | −8.534 | 1.00 | 21.73 | C |
| ATOM | 1014 | C | HIS | A | 159 | 61.175 | 22.194 | −13.092 | 1.00 | 20.62 | C |
| ATOM | 1015 | O | HIS | A | 159 | 61.558 | 21.340 | −13.883 | 1.00 | 20.59 | O |
| ATOM | 1016 | N | ASN | A | 160 | 60.433 | 23.240 | −13.463 | 1.00 | 20.97 | N |
| ATOM | 1017 | CA | ASN | A | 160 | 60.110 | 23.508 | −14.882 | 1.00 | 21.47 | C |
| ATOM | 1018 | CB | ASN | A | 160 | 59.230 | 24.754 | −15.042 | 1.00 | 21.81 | C |
| ATOM | 1019 | CG | AASN | A | 160 | 57.985 | 24.688 | −14.251 | 0.50 | 22.71 | C |
| ATOM | 1020 | CG | BASN | A | 160 | 58.366 | 24.731 | −16.318 | 0.50 | 21.42 | C |
| ATOM | 1021 | OD1 | AASN | A | 160 | 57.565 | 25.688 | −13.683 | 0.50 | 25.62 | O |
| ATOM | 1022 | OD1 | BASN | A | 160 | 58.380 | 25.680 | −17.103 | 0.50 | 21.10 | O |
| ATOM | 1023 | ND2 | AASN | A | 160 | 57.364 | 23.518 | −14.203 | 0.50 | 26.04 | N |
| ATOM | 1024 | ND2 | BASN | A | 160 | 57.598 | 23.664 | −16.506 | 0.50 | 19.99 | N |
| ATOM | 1025 | C | ASN | A | 160 | 61.353 | 23.728 | −15.731 | 1.00 | 21.48 | C |
| ATOM | 1026 | O | ASN | A | 160 | 61.344 | 23.430 | −16.925 | 1.00 | 21.00 | O |
| ATOM | 1027 | N | CYS | A | 161 | 62.404 | 24.278 | −15.115 | 1.00 | 20.77 | N |
| ATOM | 1028 | CA | CYS | A | 161 | 63.691 | 24.462 | −15.773 | 1.00 | 21.07 | C |
| ATOM | 1029 | CB | CYS | A | 161 | 64.395 | 25.716 | −15.231 | 1.00 | 20.76 | C |
| ATOM | 1030 | SG | CYS | A | 161 | 63.499 | 27.235 | −15.609 | 1.00 | 26.51 | S |
| ATOM | 1031 | C | CYS | A | 161 | 64.628 | 23.242 | −15.665 | 1.00 | 20.01 | C |
| ATOM | 1032 | O | CYS | A | 161 | 65.791 | 23.329 | −16.052 | 1.00 | 19.64 | O |
| ATOM | 1033 | N | GLY | A | 162 | 64.141 | 22.124 | −15.130 | 1.00 | 18.99 | N |
| ATOM | 1034 | CA | GLY | A | 162 | 64.965 | 20.921 | −15.005 | 1.00 | 18.24 | C |
| ATOM | 1035 | C | GLY | A | 162 | 65.968 | 20.898 | −13.850 | 1.00 | 17.99 | C |
| ATOM | 1036 | O | GLY | A | 162 | 66.963 | 20.153 | −13.878 | 1.00 | 16.70 | O |
| ATOM | 1037 | N | VAL | A | 163 | 65.696 | 21.678 | −12.805 | 1.00 | 17.86 | N |
| ATOM | 1038 | CA | VAL | A | 163 | 66.634 | 21.799 | −11.688 | 1.00 | 17.74 | C |
| ATOM | 1039 | CB | VAL | A | 163 | 67.173 | 23.251 | −11.564 | 1.00 | 18.57 | C |
| ATOM | 1040 | CG1 | VAL | A | 163 | 67.897 | 23.479 | −10.215 | 1.00 | 17.79 | C |
| ATOM | 1041 | CG2 | VAL | A | 163 | 68.078 | 23.608 | −12.766 | 1.00 | 17.89 | C |
| ATOM | 1042 | C | VAL | A | 163 | 65.970 | 21.373 | −10.374 | 1.00 | 17.54 | C |
| ATOM | 1043 | O | VAL | A | 163 | 64.851 | 21.780 | −10.071 | 1.00 | 17.67 | O |
| ATOM | 1044 | N | LEU | A | 164 | 66.673 | 20.550 | −9.612 | 1.00 | 17.14 | N |
| ATOM | 1045 | CA | LEU | A | 164 | 66.235 | 20.142 | −8.288 | 1.00 | 17.15 | C |
| ATOM | 1046 | CB | LEU | A | 164 | 66.298 | 18.614 | −8.170 | 1.00 | 17.23 | C |
| ATOM | 1047 | CG | LEU | A | 164 | 65.715 | 17.973 | −6.909 | 1.00 | 18.35 | C |
| ATOM | 1048 | CD1 | LEU | A | 164 | 64.183 | 18.038 | −6.939 | 1.00 | 18.69 | C |
| ATOM | 1049 | CD2 | LEU | A | 164 | 66.201 | 16.530 | −6.783 | 1.00 | 15.82 | C |
| ATOM | 1050 | C | LEU | A | 164 | 67.151 | 20.802 | −7.269 | 1.00 | 16.92 | C |
| ATOM | 1051 | O | LEU | A | 164 | 68.367 | 20.594 | −7.305 | 1.00 | 17.02 | O |
| ATOM | 1052 | N | HIS | A | 165 | 66.574 | 21.583 | −6.359 | 1.00 | 16.61 | N |
| ATOM | 1053 | CA | HIS | A | 165 | 67.356 | 22.378 | −5.410 | 1.00 | 15.80 | C |
| ATOM | 1054 | CB | HIS | A | 165 | 66.462 | 23.440 | −4.747 | 1.00 | 16.02 | C |
| ATOM | 1055 | CG | HIS | A | 165 | 67.212 | 24.444 | −3.924 | 1.00 | 15.04 | C |
| ATOM | 1056 | ND1 | HIS | A | 165 | 67.698 | 24.155 | −2.668 | 1.00 | 13.63 | N |
| ATOM | 1057 | CE1 | HIS | A | 165 | 68.311 | 25.218 | −2.175 | 1.00 | 14.79 | C |
| ATOM | 1058 | NE2 | HIS | A | 165 | 68.248 | 26.188 | −3.071 | 1.00 | 16.20 | N |
| ATOM | 1059 | CD2 | HIS | A | 165 | 67.571 | 25.726 | −4.182 | 1.00 | 14.88 | C |
| ATOM | 1060 | C | HIS | A | 165 | 68.065 | 21.520 | −4.352 | 1.00 | 16.12 | C |
| ATOM | 1061 | O | HIS | A | 165 | 69.281 | 21.692 | −4.112 | 1.00 | 15.47 | O |
| ATOM | 1062 | N | ARG | A | 166 | 67.301 | 20.628 | −3.708 | 1.00 | 15.81 | N |
| ATOM | 1063 | CA | ARG | A | 166 | 67.802 | 19.692 | −2.685 | 1.00 | 16.34 | C |
| ATOM | 1064 | CB | ARG | A | 166 | 68.933 | 18.830 | −3.231 | 1.00 | 16.34 | C |
| ATOM | 1065 | CG | ARG | A | 166 | 68.542 | 17.800 | −4.282 | 1.00 | 17.58 | C |
| ATOM | 1066 | CD | ARG | A | 166 | 69.743 | 17.471 | −5.131 | 1.00 | 23.63 | C |
| ATOM | 1067 | NE | ARG | A | 166 | 70.090 | 16.080 | −5.010 | 1.00 | 27.91 | N |
| ATOM | 1068 | CZ | ARG | A | 166 | 71.277 | 15.551 | −5.274 | 1.00 | 28.25 | C |
| ATOM | 1069 | NH1 | ARG | A | 166 | 72.327 | 16.299 | −5.636 | 1.00 | 26.61 | N |
| ATOM | 1070 | NH2 | ARG | A | 166 | 71.404 | 14.246 | −5.142 | 1.00 | 25.73 | N |
| ATOM | 1071 | C | ARG | A | 166 | 68.284 | 20.261 | −1.348 | 1.00 | 17.04 | C |
| ATOM | 1072 | O | ARG | A | 166 | 68.778 | 19.491 | −0.517 | 1.00 | 17.97 | O |
| ATOM | 1073 | N | ASP | A | 167 | 68.165 | 21.571 | −1.127 | 1.00 | 16.44 | N |
| ATOM | 1074 | CA | ASP | A | 167 | 68.537 | 22.157 | 0.172 | 1.00 | 17.08 | C |
| ATOM | 1075 | CB | ASP | A | 167 | 70.018 | 22.615 | 0.164 | 1.00 | 16.76 | C |
| ATOM | 1076 | CG | ASP | A | 167 | 70.639 | 22.733 | 1.576 | 1.00 | 19.52 | C |
| ATOM | 1077 | OD1 | ASP | A | 167 | 70.136 | 22.109 | 2.552 | 1.00 | 19.71 | O |
| ATOM | 1078 | OD2 | ASP | A | 167 | 71.660 | 23.441 | 1.792 | 1.00 | 20.05 | O |
| ATOM | 1079 | C | ASP | A | 167 | 67.593 | 23.303 | 0.559 | 1.00 | 16.55 | C |
| ATOM | 1080 | O | ASP | A | 167 | 68.028 | 24.327 | 1.065 | 1.00 | 17.85 | O |
| ATOM | 1081 | N | ILE | A | 168 | 66.289 | 23.130 | 0.321 | 1.00 | 16.37 | N |
| ATOM | 1082 | CA | ILE | A | 168 | 65.304 | 24.165 | 0.643 | 1.00 | 15.43 | C |
| ATOM | 1083 | CB | ILE | A | 168 | 63.919 | 23.803 | 0.061 | 1.00 | 15.49 | C |
| ATOM | 1084 | CG1 | ILE | A | 168 | 63.990 | 23.685 | −1.467 | 1.00 | 15.11 | C |
| ATOM | 1085 | CD1 | ILE | A | 168 | 62.816 | 22.891 | −2.049 | 1.00 | 15.98 | C |
| ATOM | 1086 | CG2 | ILE | A | 168 | 62.841 | 24.821 | 0.481 | 1.00 | 14.46 | C |
| ATOM | 1087 | C | ILE | A | 168 | 65.226 | 24.257 | 2.159 | 1.00 | 15.89 | C |
| ATOM | 1088 | O | ILE | A | 168 | 64.988 | 23.247 | 2.828 | 1.00 | 15.71 | O |
| ATOM | 1089 | N | LYS | A | 169 | 65.445 | 25.459 | 2.682 | 1.00 | 15.34 | N |
| ATOM | 1090 | CA | LYS | A | 169 | 65.458 | 25.724 | 4.116 | 1.00 | 15.97 | C |
| ATOM | 1091 | CB | LYS | A | 169 | 66.666 | 25.055 | 4.793 | 1.00 | 16.17 | C |
| ATOM | 1092 | CG | LYS | A | 169 | 68.018 | 25.601 | 4.321 | 1.00 | 18.35 | C |
| ATOM | 1093 | CD | LYS | A | 169 | 69.165 | 24.636 | 4.595 | 1.00 | 21.69 | C |
| ATOM | 1094 | CE | LYS | A | 169 | 69.449 | 24.497 | 6.073 | 1.00 | 23.35 | C |
| ATOM | 1095 | NZ | LYS | A | 169 | 70.883 | 24.061 | 6.239 | 1.00 | 24.28 | N |
| ATOM | 1096 | C | LYS | A | 169 | 65.542 | 27.234 | 4.314 | 1.00 | 15.57 | C |
| ATOM | 1097 | O | LYS | A | 169 | 65.954 | 27.971 | 3.392 | 1.00 | 15.50 | O |
| ATOM | 1098 | N | ASP | A | 170 | 65.213 | 27.676 | 5.526 | 1.00 | 15.04 | N |
| ATOM | 1099 | CA | ASP | A | 170 | 65.179 | 29.090 | 5.868 | 1.00 | 15.81 | C |
| ATOM | 1100 | CB | ASP | A | 170 | 64.849 | 29.284 | 7.358 | 1.00 | 15.73 | C |
| ATOM | 1101 | CG | ASP | A | 170 | 65.734 | 28.457 | 8.295 | 1.00 | 18.50 | C |
| ATOM | 1102 | OD1 | ASP | A | 170 | 66.780 | 27.869 | 7.880 | 1.00 | 19.79 | O |
| ATOM | 1103 | OD2 | ASP | A | 170 | 65.450 | 28.361 | 9.509 | 1.00 | 21.07 | O |
| ATOM | 1104 | C | ASP | A | 170 | 66.433 | 29.880 | 5.468 | 1.00 | 16.21 | C |
| ATOM | 1105 | O | ASP | A | 170 | 66.321 | 30.954 | 4.874 | 1.00 | 16.22 | O |
| ATOM | 1106 | N | GLU | A | 171 | 67.607 | 29.341 | 5.792 | 1.00 | 16.57 | N |
| ATOM | 1107 | CA | GLU | A | 171 | 68.918 | 29.951 | 5.480 | 1.00 | 17.89 | C |
| ATOM | 1108 | CB | GLU | A | 171 | 70.040 | 28.959 | 5.796 | 1.00 | 18.41 | C |
| ATOM | 1109 | CG | GLU | A | 171 | 70.770 | 29.167 | 7.082 | 1.00 | 24.87 | C |
| ATOM | 1110 | CD | GLU | A | 171 | 71.735 | 28.024 | 7.352 | 1.00 | 29.19 | C |
| ATOM | 1111 | OE1 | GLU | A | 171 | 72.124 | 27.876 | 8.521 | 1.00 | 34.95 | O |
| ATOM | 1112 | OE2 | GLU | A | 171 | 72.072 | 27.259 | 6.407 | 1.00 | 30.15 | O |
| ATOM | 1113 | C | GLU | A | 171 | 69.096 | 30.224 | 3.998 | 1.00 | 16.68 | C |
| ATOM | 1114 | O | GLU | A | 171 | 69.853 | 31.125 | 3.626 | 1.00 | 15.52 | O |
| ATOM | 1115 | N | ASN | A | 172 | 68.468 | 29.391 | 3.171 | 1.00 | 15.92 | N |
| ATOM | 1116 | CA | ASN | A | 172 | 68.601 | 29.487 | 1.707 | 1.00 | 15.37 | C |
| ATOM | 1117 | CB | ASN | A | 172 | 68.806 | 28.111 | 1.093 | 1.00 | 14.94 | C |
| ATOM | 1118 | CG | ASN | A | 172 | 70.122 | 27.517 | 1.493 | 1.00 | 15.14 | C |
| ATOM | 1119 | OD1 | ASN | A | 172 | 71.047 | 28.264 | 1.760 | 1.00 | 15.76 | O |
| ATOM | 1120 | ND2 | ASN | A | 172 | 70.218 | 26.188 | 1.567 | 1.00 | 13.68 | N |
| ATOM | 1121 | C | ASN | A | 172 | 67.454 | 30.228 | 1.026 | 1.00 | 15.23 | C |
| ATOM | 1122 | O | ASN | A | 172 | 67.198 | 30.038 | −0.154 | 1.00 | 15.24 | O |
| ATOM | 1123 | N | ILE | A | 173 | 66.799 | 31.102 | 1.778 | 1.00 | 15.43 | N |
| ATOM | 1124 | CA | ILE | A | 173 | 65.714 | 31.920 | 1.252 | 1.00 | 15.78 | C |
| ATOM | 1125 | CB | ILE | A | 173 | 64.350 | 31.416 | 1.775 | 1.00 | 15.76 | C |
| ATOM | 1126 | CG1 | ILE | A | 173 | 64.066 | 29.987 | 1.287 | 1.00 | 16.39 | C |
| ATOM | 1127 | CD1 | ILE | A | 173 | 62.948 | 29.264 | 2.080 | 1.00 | 14.60 | C |
| ATOM | 1128 | CG2 | ILE | A | 173 | 63.211 | 32.396 | 1.379 | 1.00 | 15.41 | C |
| ATOM | 1129 | C | ILE | A | 173 | 65.947 | 33.363 | 1.701 | 1.00 | 15.85 | C |
| ATOM | 1130 | O | ILE | A | 173 | 66.149 | 33.622 | 2.884 | 1.00 | 15.12 | O |
| ATOM | 1131 | N | LEU | A | 174 | 65.914 | 34.285 | 0.741 | 1.00 | 16.29 | N |
| ATOM | 1132 | CA | LEU | A | 174 | 66.139 | 35.707 | 0.992 | 1.00 | 16.81 | C |
| ATOM | 1133 | CB | LEU | A | 174 | 67.035 | 36.307 | −0.105 | 1.00 | 16.84 | C |
| ATOM | 1134 | CG | LEU | A | 174 | 68.479 | 35.805 | −0.189 | 1.00 | 17.08 | C |
| ATOM | 1135 | CD1 | LEU | A | 174 | 69.217 | 36.628 | −1.214 | 1.00 | 16.20 | C |
| ATOM | 1136 | CD2 | LEU | A | 174 | 69.187 | 35.865 | 1.153 | 1.00 | 16.61 | C |
| ATOM | 1137 | C | LEU | A | 174 | 64.816 | 36.419 | 0.956 | 1.00 | 17.35 | C |
| ATOM | 1138 | O | LEU | A | 174 | 63.963 | 36.085 | 0.127 | 1.00 | 17.94 | O |
| ATOM | 1139 | N | ILE | A | 175 | 64.641 | 37.387 | 1.850 | 1.00 | 17.74 | N |
| ATOM | 1140 | CA | ILE | A | 175 | 63.470 | 38.255 | 1.833 | 1.00 | 18.68 | C |
| ATOM | 1141 | CB | ILE | A | 175 | 62.818 | 38.360 | 3.226 | 1.00 | 18.50 | C |
| ATOM | 1142 | CG1 | ILE | A | 175 | 62.456 | 36.976 | 3.794 | 1.00 | 18.87 | C |
| ATOM | 1143 | CD1 | ILE | A | 175 | 62.302 | 36.995 | 5.327 | 1.00 | 18.86 | C |
| ATOM | 1144 | CG2 | ILE | A | 175 | 61.576 | 39.278 | 3.167 | 1.00 | 18.77 | C |
| ATOM | 1145 | C | ILE | A | 175 | 63.902 | 39.649 | 1.389 | 1.00 | 19.58 | C |
| ATOM | 1146 | O | ILE | A | 175 | 64.664 | 40.322 | 2.095 | 1.00 | 19.31 | O |
| ATOM | 1147 | N | ASP | A | 176 | 63.412 | 40.074 | 0.228 | 1.00 | 20.23 | N |
| ATOM | 1148 | CA | ASP | A | 176 | 63.581 | 41.449 | −0.230 | 1.00 | 21.84 | C |
| ATOM | 1149 | CB | ASP | A | 176 | 63.315 | 41.541 | −1.739 | 1.00 | 22.01 | C |
| ATOM | 1150 | CG | ASP | A | 176 | 63.414 | 42.967 | −2.280 | 1.00 | 23.60 | C |
| ATOM | 1151 | OD1 | ASP | A | 176 | 63.243 | 43.920 | −1.502 | 1.00 | 23.50 | O |
| ATOM | 1152 | OD2 | ASP | A | 176 | 63.625 | 43.217 | −3.482 | 1.00 | 24.73 | O |
| ATOM | 1153 | C | ASP | A | 176 | 62.588 | 42.286 | 0.587 | 1.00 | 22.79 | C |
| ATOM | 1154 | O | ASP | A | 176 | 61.387 | 42.316 | 0.297 | 1.00 | 22.81 | O |
| ATOM | 1155 | N | LEU | A | 177 | 63.102 | 42.924 | 1.634 | 1.00 | 23.58 | N |
| ATOM | 1156 | CA | LEU | A | 177 | 62.271 | 43.537 | 2.660 | 1.00 | 24.93 | C |
| ATOM | 1157 | CB | LEU | A | 177 | 63.132 | 44.012 | 3.835 | 1.00 | 25.33 | C |
| ATOM | 1158 | CG | LEU | A | 111 | 63.764 | 42.908 | 4.700 | 1.00 | 25.91 | C |
| ATOM | 1159 | CD1 | LEU | A | 111 | 64.830 | 43.473 | 5.621 | 1.00 | 26.41 | C |
| ATOM | 1160 | CD2 | LEU | A | 111 | 62.715 | 42.118 | 5.504 | 1.00 | 27.00 | C |
| ATOM | 1161 | C | LEU | A | 177 | 61.345 | 44.658 | 2.164 | 1.00 | 25.53 | C |
| ATOM | 1162 | O | LEU | A | 177 | 60.231 | 44.789 | 2.661 | 1.00 | 26.29 | O |
| ATOM | 1163 | N | ASN | A | 178 | 61.789 | 45.433 | 1.177 | 1.00 | 25.87 | N |
| ATOM | 1164 | CA | ASN | A | 178 | 60.985 | 46.525 | 0.607 | 1.00 | 26.30 | C |
| ATOM | 1165 | CB | ASN | A | 178 | 61.875 | 47.492 | −0.187 | 1.00 | 26.74 | C |
| ATOM | 1166 | CG | ASN | A | 178 | 62.544 | 48.534 | 0.690 | 1.00 | 28.73 | C |
| ATOM | 1167 | OD1 | ASN | A | 178 | 62.308 | 48.600 | 1.904 | 1.00 | 31.92 | O |
| ATOM | 1168 | ND2 | ASN | A | 178 | 63.382 | 49.361 | 0.078 | 1.00 | 30.86 | N |
| ATOM | 1169 | C | ASN | A | 178 | 59.857 | 46.042 | −0.307 | 1.00 | 26.01 | C |
| ATOM | 1170 | O | ASN | A | 178 | 58.771 | 46.630 | −0.338 | 1.00 | 25.84 | O |
| ATOM | 1171 | N | ARG | A | 179 | 60.134 | 44.986 | −1.066 | 1.00 | 25.22 | N |
| ATOM | 1172 | CA | ARG | A | 179 | 59.202 | 44.497 | −2.073 | 1.00 | 25.06 | C |
| ATOM | 1173 | CB | ARG | A | 179 | 59.951 | 44.089 | −3.340 | 1.00 | 25.43 | C |
| ATOM | 1174 | CG | ARG | A | 179 | 60.482 | 45.270 | −4.157 | 1.00 | 26.18 | C |
| ATOM | 1175 | CD | ARG | A | 179 | 61.170 | 44.845 | −5.426 | 1.00 | 29.81 | C |
| ATOM | 1176 | NE | ARG | A | 179 | 61.712 | 45.951 | −6.219 | 1.00 | 33.67 | N |
| ATOM | 1177 | CZ | ARG | A | 179 | 60.987 | 46.869 | −6.859 | 1.00 | 35.31 | C |
| ATOM | 1178 | NH1 | ARG | A | 179 | 59.658 | 46.854 | −6.805 | 1.00 | 36.83 | N |
| ATOM | 1179 | NH2 | ARG | A | 179 | 61.598 | 47.816 | −7.559 | 1.00 | 36.82 | N |
| ATOM | 1180 | C | ARG | A | 179 | 58.330 | 43.355 | −1.574 | 1.00 | 24.41 | C |
| ATOM | 1181 | O | ARG | A | 179 | 57.345 | 43.004 | −2.221 | 1.00 | 24.91 | O |
| ATOM | 1182 | N | GLY | A | 180 | 58.675 | 42.786 | −0.421 | 1.00 | 23.59 | N |
| ATOM | 1183 | CA | GLY | A | 180 | 57.977 | 41.611 | 0.083 | 1.00 | 22.56 | C |
| ATOM | 1184 | C | GLY | A | 180 | 58.160 | 40.359 | −0.779 | 1.00 | 21.90 | C |
| ATOM | 1185 | O | GLY | A | 180 | 57.320 | 39.456 | −0.754 | 1.00 | 21.19 | O |
| ATOM | 1186 | N | GLU | A | 181 | 59.263 | 40.310 | −1.524 | 1.00 | 21.18 | N |
| ATOM | 1187 | CA | GLU | A | 181 | 59.542 | 39.216 | −2.462 | 1.00 | 21.26 | C |
| ATOM | 1188 | CB | GLU | A | 181 | 60.001 | 39.767 | −3.815 | 1.00 | 20.99 | C |
| ATOM | 1189 | CG | GLU | A | 181 | 58.883 | 40.426 | −4.598 | 1.00 | 22.33 | C |
| ATOM | 1190 | CD | GLU | A | 181 | 59.360 | 41.165 | −5.827 | 1.00 | 24.38 | C |
| ATOM | 1191 | OE1 | GLU | A | 181 | 60.493 | 40.911 | −6.310 | 1.00 | 25.63 | O |
| ATOM | 1192 | OE2 | GLU | A | 181 | 58.578 | 42.011 | −6.317 | 1.00 | 26.99 | O |
| ATOM | 1193 | C | GLU | A | 181 | 60.613 | 38.281 | −1.928 | 1.00 | 20.80 | C |
| ATOM | 1194 | O | GLU | A | 181 | 61.659 | 38.735 | −1.467 | 1.00 | 20.59 | O |
| ATOM | 1195 | N | LEU | A | 182 | 60.348 | 36.979 | −2.002 | 1.00 | 20.60 | N |
| ATOM | 1196 | CA | LEU | A | 182 | 61.297 | 35.963 | −1.555 | 1.00 | 20.56 | C |
| ATOM | 1197 | CB | LEU | A | 182 | 60.570 | 34.791 | −0.891 | 1.00 | 20.37 | C |
| ATOM | 1198 | CG | LEU | A | 182 | 60.517 | 34.821 | 0.631 | 1.00 | 21.15 | C |
| ATOM | 1199 | CD1 | LEU | A | 182 | 59.818 | 36.090 | 1.096 | 1.00 | 22.41 | C |
| ATOM | 1200 | CD2 | LEU | A | 182 | 59.801 | 33.559 | 1.145 | 1.00 | 19.08 | C |
| ATOM | 1201 | C | LEU | A | 182 | 62.118 | 35.439 | −2.725 | 1.00 | 20.73 | C |
| ATOM | 1202 | O | LEU | A | 182 | 61.612 | 35.341 | −3.849 | 1.00 | 20.32 | O |
| ATOM | 1203 | N | LYS | A | 183 | 63.372 | 35.096 | −2.442 | 1.00 | 20.55 | N |
| ATOM | 1204 | CA | LYS | A | 183 | 64.313 | 34.617 | −3.448 | 1.00 | 20.89 | C |
| ATOM | 1205 | CB | LYS | A | 183 | 65.374 | 35.692 | −3.759 | 1.00 | 21.35 | C |
| ATOM | 1206 | CG | LYS | A | 183 | 64.883 | 36.741 | −4.750 | 1.00 | 24.94 | C |
| ATOM | 1207 | CD | LYS | A | 183 | 65.757 | 37.973 | −4.773 | 1.00 | 26.84 | C |
| ATOM | 1208 | CE | LYS | A | 183 | 65.777 | 38.639 | −6.149 | 1.00 | 30.89 | C |
| ATOM | 1209 | NZ | LYS | A | 183 | 64.436 | 38.886 | −6.765 | 1.00 | 30.16 | N |
| ATOM | 1210 | C | LYS | A | 183 | 65.005 | 33.373 | −2.927 | 1.00 | 19.72 | C |
| ATOM | 1211 | O | LYS | A | 183 | 65.572 | 33.384 | −1.844 | 1.00 | 19.28 | O |
| ATOM | 1212 | N | LEU | A | 184 | 64.960 | 32.317 | −3.725 | 1.00 | 18.78 | N |
| ATOM | 1213 | CA | LEU | A | 184 | 65.689 | 31.088 | −3.462 | 1.00 | 18.80 | C |
| ATOM | 1214 | CB | LEU | A | 184 | 65.057 | 29.957 | −4.278 | 1.00 | 18.65 | C |
| ATOM | 1215 | CG | LEU | A | 184 | 65.182 | 28.479 | −3.925 | 1.00 | 22.15 | C |
| ATOM | 1216 | CD1 | LEU | A | 184 | 64.840 | 28.132 | −2.445 | 1.00 | 22.78 | C |
| ATOM | 1217 | CD2 | LEU | A | 184 | 64.302 | 27.643 | −4.894 | 1.00 | 19.46 | C |
| ATOM | 1218 | C | LEU | A | 184 | 67.166 | 31.270 | −3.827 | 1.00 | 17.97 | C |
| ATOM | 1219 | O | LEU | A | 184 | 67.500 | 31.813 | −4.895 | 1.00 | 17.51 | O |
| ATOM | 1220 | N | ILE | A | 185 | 68.048 | 30.840 | −2.932 | 1.00 | 16.83 | N |
| ATOM | 1221 | CA | ILE | A | 185 | 69.482 | 30.849 | −3.204 | 1.00 | 16.25 | C |
| ATOM | 1222 | CB | ILE | A | 185 | 70.215 | 31.922 | −2.348 | 1.00 | 16.56 | C |
| ATOM | 1223 | CG1 | ILE | A | 185 | 69.892 | 31.720 | −0.859 | 1.00 | 15.91 | C |
| ATOM | 1224 | CD1 | ILE | A | 185 | 70.811 | 32.451 | 0.107 | 1.00 | 16.54 | C |
| ATOM | 1225 | CG2 | ILE | A | 185 | 69.924 | 33.333 | −2.865 | 1.00 | 15.65 | C |
| ATOM | 1226 | C | ILE | A | 185 | 70.098 | 29.504 | −2.880 | 1.00 | 16.22 | C |
| ATOM | 1227 | O | ILE | A | 185 | 69.411 | 28.607 | −2.380 | 1.00 | 16.13 | O |
| ATOM | 1228 | N | ASP | A | 186 | 71.409 | 29.408 | −3.127 | 1.00 | 15.92 | N |
| ATOM | 1229 | CA | ASP | A | 186 | 72.254 | 28.263 | −2.788 | 1.00 | 15.84 | C |
| ATOM | 1230 | CB | ASP | A | 186 | 72.344 | 28.039 | −1.269 | 1.00 | 16.13 | C |
| ATOM | 1231 | CG | ASP | A | 186 | 73.351 | 26.972 | −0.898 | 1.00 | 15.73 | C |
| ATOM | 1232 | OD1 | ASP | A | 186 | 73.977 | 26.372 | −1.791 | 1.00 | 17.28 | O |
| ATOM | 1233 | OD2 | ASP | A | 186 | 73.571 | 26.621 | 0.268 | 1.00 | 16.60 | O |
| ATOM | 1234 | C | ASP | A | 186 | 71.875 | 26.980 | −3.505 | 1.00 | 17.14 | C |
| ATOM | 1235 | O | ASP | A | 186 | 71.185 | 26.128 | −2.968 | 1.00 | 17.68 | O |
| ATOM | 1236 | N | PHE | A | 187 | 72.372 | 26.828 | −4.721 | 1.00 | 17.98 | N |
| ATOM | 1237 | CA | PHE | A | 187 | 72.163 | 25.603 | −5.459 | 1.00 | 19.11 | C |
| ATOM | 1238 | CB | PHE | A | 187 | 71.813 | 25.935 | −6.905 | 1.00 | 19.00 | C |
| ATOM | 1239 | CG | PHE | A | 187 | 70.462 | 26.572 | −7.042 | 1.00 | 18.98 | C |
| ATOM | 1240 | CD1 | PHE | A | 187 | 70.277 | 27.914 | −6.731 | 1.00 | 17.58 | C |
| ATOM | 1241 | CE1 | PHE | A | 187 | 69.010 | 28.508 | −6.832 | 1.00 | 19.96 | C |
| ATOM | 1242 | CZ | PHE | A | 187 | 67.917 | 27.743 | −7.260 | 1.00 | 19.45 | C |
| ATOM | 1243 | CE2 | PHE | A | 187 | 68.094 | 26.402 | −7.575 | 1.00 | 18.98 | C |
| ATOM | 1244 | CD2 | PHE | A | 187 | 69.366 | 25.817 | −7.452 | 1.00 | 19.42 | C |
| ATOM | 1245 | C | PHE | A | 187 | 73.367 | 24.669 | −5.342 | 1.00 | 19.81 | C |
| ATOM | 1246 | O | PHE | A | 187 | 73.540 | 23.776 | −6.162 | 1.00 | 20.32 | O |
| ATOM | 1247 | N | GLY | A | 188 | 74.157 | 24.864 | −4.285 | 1.00 | 20.15 | N |
| ATOM | 1248 | CA | GLY | A | 188 | 75.355 | 24.074 | −4.027 | 1.00 | 20.64 | C |
| ATOM | 1249 | C | GLY | A | 188 | 75.130 | 22.581 | −3.824 | 1.00 | 20.89 | C |
| ATOM | 1250 | O | GLY | A | 188 | 76.061 | 21.795 | −4.008 | 1.00 | 20.70 | O |
| ATOM | 1251 | N | SER | A | 189 | 73.904 | 22.186 | −3.460 | 1.00 | 20.61 | N |
| ATOM | 1252 | CA | SER | A | 189 | 73.585 | 20.774 | −3.205 | 1.00 | 20.36 | C |
| ATOM | 1253 | CB | SER | A | 189 | 72.891 | 20.598 | −1.843 | 1.00 | 20.68 | C |
| ATOM | 1254 | OG | SER | A | 189 | 73.701 | 21.035 | −0.766 | 1.00 | 20.77 | O |
| ATOM | 1255 | C | SER | A | 189 | 72.668 | 20.218 | −4.276 | 1.00 | 20.23 | C |
| ATOM | 1256 | O | SER | A | 189 | 72.229 | 19.079 | −4.181 | 1.00 | 19.67 | O |
| ATOM | 1257 | N | GLY | A | 190 | 72.359 | 21.040 | −5.273 | 1.00 | 19.66 | N |
| ATOM | 1258 | CA | GLY | A | 190 | 71.362 | 20.701 | −6.256 | 1.00 | 20.11 | C |
| ATOM | 1259 | C | GLY | A | 190 | 71.779 | 19.655 | −7.282 | 1.00 | 20.35 | C |
| ATOM | 1260 | O | GLY | A | 190 | 72.924 | 19.203 | −7.309 | 1.00 | 20.22 | O |
| ATOM | 1261 | N | ALA | A | 191 | 70.830 | 19.271 | −8.122 | 1.00 | 19.81 | N |
| ATOM | 1262 | CA | ALA | A | 191 | 71.085 | 18.339 | −9.201 | 1.00 | 20.13 | C |
| ATOM | 1263 | CB | ALA | A | 191 | 70.902 | 16.875 | −8.722 | 1.00 | 20.04 | C |
| ATOM | 1264 | C | ALA | A | 191 | 70.130 | 18.652 | −10.323 | 1.00 | 20.23 | C |
| ATOM | 1265 | O | ALA | A | 191 | 69.126 | 19.344 | −10.122 | 1.00 | 20.11 | O |
| ATOM | 1266 | N | LEU | A | 192 | 70.446 | 18.144 | −11.512 | 1.00 | 20.33 | N |
| ATOM | 1267 | CA | LEU | A | 192 | 69.504 | 18.154 | −12.617 | 1.00 | 20.48 | C |
| ATOM | 1268 | CB | LEU | A | 192 | 70.160 | 17.538 | −13.870 | 1.00 | 20.65 | C |
| ATOM | 1269 | CG | LEU | A | 192 | 71.394 | 18.241 | −14.460 | 1.00 | 21.30 | C |
| ATOM | 1270 | CD1 | LEU | A | 192 | 72.025 | 17.441 | −15.650 | 1.00 | 24.01 | C |
| ATOM | 1271 | CD2 | LEU | A | 192 | 71.028 | 19.649 | −14.922 | 1.00 | 20.92 | C |
| ATOM | 1272 | C | LEU | A | 192 | 68.301 | 17.329 | −12.171 | 1.00 | 20.62 | C |
| ATOM | 1273 | O | LEU | A | 192 | 68.472 | 16.302 | −11.520 | 1.00 | 20.87 | O |
| ATOM | 1274 | N | LEU | A | 193 | 67.092 | 17.787 | −12.488 | 1.00 | 20.82 | N |
| ATOM | 1275 | CA | LEU | A | 193 | 65.883 | 17.033 | −12.163 | 1.00 | 20.67 | C |
| ATOM | 1276 | CB | LEU | A | 193 | 64.626 | 17.901 | −12.333 | 1.00 | 20.90 | C |
| ATOM | 1277 | CG | LEU | A | 193 | 63.271 | 17.308 | −11.915 | 1.00 | 21.37 | C |
| ATOM | 1278 | CD1 | LEU | A | 193 | 63.216 | 16.979 | −10.428 | 1.00 | 19.30 | C |
| ATOM | 1279 | CD2 | LEU | A | 193 | 62.103 | 18.226 | −12.303 | 1.00 | 23.02 | C |
| ATOM | 1280 | C | LEU | A | 193 | 65.770 | 15.784 | −13.042 | 1.00 | 20.94 | C |
| ATOM | 1281 | O | LEU | A | 193 | 66.005 | 15.837 | −14.250 | 1.00 | 20.46 | O |
| ATOM | 1282 | N | LYS | A | 194 | 65.403 | 14.666 | −12.423 | 1.00 | 20.70 | N |
| ATOM | 1283 | CA | LYS | A | 194 | 65.191 | 13.411 | −13.140 | 1.00 | 20.09 | C |
| ATOM | 1284 | CB | LYS | A | 194 | 66.464 | 12.559 | −13.094 | 1.00 | 20.03 | C |
| ATOM | 1285 | CG | LYS | A | 194 | 66.780 | 11.998 | −11.717 | 1.00 | 18.07 | C |
| ATOM | 1286 | CD | LYS | A | 194 | 68.169 | 11.391 | −11.691 | 1.00 | 19.71 | C |
| ATOM | 1287 | CE | LYS | A | 194 | 68.381 | 10.652 | −10.385 | 1.00 | 20.42 | C |
| ATOM | 1288 | NZ | LYS | A | 194 | 69.586 | 9.789 | −10.403 | 1.00 | 19.70 | N |
| ATOM | 1289 | C | LYS | A | 194 | 64.025 | 12.658 | −12.505 | 1.00 | 19.94 | C |
| ATOM | 1290 | O | LYS | A | 194 | 63.669 | 12.913 | −11.349 | 1.00 | 19.51 | O |
| ATOM | 1291 | N | ASP | A | 195 | 63.456 | 11.722 | −13.260 | 1.00 | 19.71 | N |
| ATOM | 1292 | CA | ASP | A | 195 | 62.308 | 10.943 | −12.808 | 1.00 | 19.96 | C |
| ATOM | 1293 | CB | ASP | A | 195 | 61.352 | 10.706 | −13.972 | 1.00 | 20.12 | C |
| ATOM | 1294 | CG | ASP | A | 195 | 60.843 | 12.005 | −14.573 | 1.00 | 21.01 | C |
| ATOM | 1295 | OD1 | ASP | A | 195 | 60.213 | 12.792 | −13.832 | 1.00 | 21.96 | O |
| ATOM | 1296 | OD2 | ASP | A | 195 | 61.028 | 12.311 | −15.770 | 1.00 | 22.33 | O |
| ATOM | 1297 | C | ASP | A | 195 | 62.684 | 9.613 | −12.166 | 1.00 | 20.21 | C |
| ATOM | 1298 | O | ASP | A | 195 | 61.811 | 8.866 | −11.740 | 1.00 | 20.16 | O |
| ATOM | 1299 | N | THR | A | 196 | 63.979 | 9.324 | −12.111 | 1.00 | 20.34 | N |
| ATOM | 1300 | CA | THR | A | 196 | 64.459 | 8.086 | −11.519 | 1.00 | 20.80 | C |
| ATOM | 1301 | CB | THR | A | 196 | 65.550 | 7.433 | −12.402 | 1.00 | 20.79 | C |
| ATOM | 1302 | OG1 | THR | A | 196 | 66.489 | 8.431 | −12.832 | 1.00 | 19.71 | O |
| ATOM | 1303 | CG2 | THR | A | 196 | 64.942 | 6.891 | −13.701 | 1.00 | 20.94 | C |
| ATOM | 1304 | C | THR | A | 196 | 64.997 | 8.357 | −10.132 | 1.00 | 21.32 | C |
| ATOM | 1305 | O | THR | A | 196 | 65.059 | 9.515 | −9.686 | 1.00 | 21.17 | O |
| ATOM | 1306 | N | VAL | A | 197 | 65.387 | 7.290 | −9.447 | 1.00 | 21.71 | N |
| ATOM | 1307 | CA | VAL | A | 197 | 65.741 | 7.385 | −8.039 | 1.00 | 22.75 | C |
| ATOM | 1308 | CB | VAL | A | 197 | 65.661 | 5.983 | −7.364 | 1.00 | 22.95 | C |
| ATOM | 1309 | CG1 | VAL | A | 197 | 66.823 | 5.098 | −7.798 | 1.00 | 24.30 | C |
| ATOM | 1310 | CG2 | VAL | A | 197 | 65.592 | 6.094 | −5.849 | 1.00 | 23.66 | C |
| ATOM | 1311 | C | VAL | A | 197 | 67.102 | 8.074 | −7.834 | 1.00 | 22.86 | C |
| ATOM | 1312 | O | VAL | A | 197 | 68.044 | 7.862 | −8.611 | 1.00 | 23.08 | O |
| ATOM | 1313 | N | TYR | A | 198 | 67.176 | 8.939 | −6.823 | 1.00 | 22.60 | N |
| ATOM | 1314 | CA | TYR | A | 198 | 68.441 | 9.506 | −6.375 | 1.00 | 22.42 | C |
| ATOM | 1315 | CB | TYR | A | 198 | 68.242 | 10.922 | −5.829 | 1.00 | 21.98 | C |
| ATOM | 1316 | CG | TYR | A | 198 | 67.927 | 11.966 | −6.869 | 1.00 | 19.83 | C |
| ATOM | 1317 | CD1 | TYR | A | 198 | 66.610 | 12.197 | −7.262 | 1.00 | 18.45 | C |
| ATOM | 1318 | CE1 | TYR | A | 198 | 66.301 | 13.147 | −8.205 | 1.00 | 16.56 | C |
| ATOM | 1319 | CZ | TYR | A | 198 | 67.307 | 13.909 | −8.772 | 1.00 | 17.06 | C |
| ATOM | 1320 | OH | TYR | A | 198 | 66.949 | 14.848 | −9.713 | 1.00 | 15.18 | O |
| ATOM | 1321 | CE2 | TYR | A | 198 | 68.641 | 13.708 | −8.410 | 1.00 | 16.87 | C |
| ATOM | 1322 | CD2 | TYR | A | 198 | 68.942 | 12.732 | −7.455 | 1.00 | 18.51 | C |
| ATOM | 1323 | C | TYR | A | 198 | 69.010 | 8.631 | −5.266 | 1.00 | 22.99 | C |
| ATOM | 1324 | O | TYR | A | 198 | 68.273 | 8.209 | −4.363 | 1.00 | 22.53 | O |
| ATOM | 1325 | N | THR | A | 199 | 70.314 | 8.370 | −5.337 | 1.00 | 23.94 | N |
| ATOM | 1326 | CA | THR | A | 199 | 71.034 | 7.617 | −4.302 | 1.00 | 25.22 | C |
| ATOM | 1327 | CB | THR | A | 199 | 71.694 | 6.331 | −4.880 | 1.00 | 25.19 | C |
| ATOM | 1328 | OG1 | THR | A | 199 | 72.604 | 6.688 | −5.923 | 1.00 | 25.62 | O |
| ATOM | 1329 | CG2 | THR | A | 199 | 70.681 | 5.427 | −5.571 | 1.00 | 25.21 | C |
| ATOM | 1330 | C | THR | A | 199 | 72.111 | 8.475 | −3.635 | 1.00 | 26.00 | C |
| ATOM | 1331 | O | THR | A | 199 | 72.881 | 7.982 | −2.818 | 1.00 | 25.92 | O |
| ATOM | 1332 | N | ASP | A | 200 | 72.170 | 9.752 | −4.007 | 1.00 | 27.25 | N |
| ATOM | 1333 | CA | ASP | A | 200 | 73.121 | 10.694 | −3.424 | 1.00 | 28.69 | C |
| ATOM | 1334 | CB | ASP | A | 200 | 74.132 | 11.197 | −4.477 | 1.00 | 29.29 | C |
| ATOM | 1335 | CG | ASP | A | 200 | 73.490 | 12.085 | −5.559 | 1.00 | 32.11 | C |
| ATOM | 1336 | OD1 | ASP | A | 200 | 73.879 | 13.277 | −5.649 | 1.00 | 34.36 | O |
| ATOM | 1337 | OD2 | ASP | A | 200 | 72.609 | 11.686 | −6.370 | 1.00 | 34.31 | O |
| ATOM | 1338 | C | ASP | A | 200 | 72.374 | 11.855 | −2.788 | 1.00 | 28.79 | C |
| ATOM | 1339 | O | ASP | A | 200 | 71.327 | 12.278 | −3.283 | 1.00 | 28.34 | O |
| ATOM | 1340 | N | PHE | A | 201 | 72.904 | 12.350 | −1.677 | 1.00 | 29.39 | N |
| ATOM | 1341 | CA | PHE | A | 201 | 72.328 | 13.501 | −1.000 | 1.00 | 30.23 | C |
| ATOM | 1342 | CB | PHE | A | 201 | 71.140 | 13.077 | −0.140 | 1.00 | 29.94 | C |
| ATOM | 1343 | CG | PHE | A | 201 | 70.534 | 14.196 | 0.660 | 1.00 | 28.00 | C |
| ATOM | 1344 | CD1 | PHE | A | 201 | 69.676 | 15.109 | 0.062 | 1.00 | 27.14 | C |
| ATOM | 1345 | CE1 | PHE | A | 201 | 69.104 | 16.143 | 0.791 | 1.00 | 27.27 | C |
| ATOM | 1346 | CZ | PHE | A | 201 | 69.381 | 16.266 | 2.148 | 1.00 | 27.00 | C |
| ATOM | 1347 | CE2 | PHE | A | 201 | 70.244 | 15.357 | 2.763 | 1.00 | 28.69 | C |
| ATOM | 1348 | CD2 | PHE | A | 201 | 70.811 | 14.322 | 2.012 | 1.00 | 28.58 | C |
| ATOM | 1349 | C | PHE | A | 201 | 73.381 | 14.192 | −0.146 | 1.00 | 31.40 | C |
| ATOM | 1350 | O | PHE | A | 201 | 74.097 | 13.542 | 0.614 | 1.00 | 31.87 | O |
| ATOM | 1351 | N | ASP | A | 202 | 73.449 | 15.512 | −0.274 | 1.00 | 32.09 | N |
| ATOM | 1352 | CA | ASP | A | 202 | 74.428 | 16.314 | 0.432 | 1.00 | 33.18 | C |
| ATOM | 1353 | CB | ASP | A | 202 | 75.581 | 16.677 | −0.510 | 1.00 | 34.28 | C |
| ATOM | 1354 | CG | ASP | A | 202 | 76.918 | 16.282 | 0.054 | 1.00 | 37.84 | C |
| ATOM | 1355 | OD1 | ASP | A | 202 | 77.292 | 15.090 | −0.089 | 1.00 | 42.28 | O |
| ATOM | 1356 | OD2 | ASP | A | 202 | 77.655 | 17.087 | 0.671 | 1.00 | 41.24 | O |
| ATOM | 1357 | C | ASP | A | 202 | 73.823 | 17.576 | 1.024 | 1.00 | 32.23 | C |
| ATOM | 1358 | O | ASP | A | 202 | 74.550 | 18.471 | 1.451 | 1.00 | 32.66 | O |
| ATOM | 1359 | N | GLY | A | 203 | 72.494 | 17.648 | 1.049 | 1.00 | 31.25 | N |
| ATOM | 1360 | CA | GLY | A | 203 | 71.801 | 18.764 | 1.677 | 1.00 | 29.41 | C |
| ATOM | 1361 | C | GLY | A | 203 | 71.721 | 18.616 | 3.189 | 1.00 | 28.69 | C |
| ATOM | 1362 | O | GLY | A | 203 | 72.489 | 17.863 | 3.792 | 1.00 | 28.32 | O |
| ATOM | 1363 | N | THR | A | 204 | 70.770 | 19.324 | 3.800 | 1.00 | 28.21 | N |
| ATOM | 1364 | CA | THR | A | 204 | 70.628 | 19.353 | 5.257 | 1.00 | 27.26 | C |
| ATOM | 1365 | CB | THR | A | 204 | 69.950 | 20.656 | 5.702 | 1.00 | 26.96 | C |
| ATOM | 1366 | OG1 | THR | A | 204 | 70.654 | 21.769 | 5.142 | 1.00 | 26.09 | O |
| ATOM | 1367 | CG2 | THR | A | 204 | 70.103 | 20.855 | 7.222 | 1.00 | 25.58 | C |
| ATOM | 1368 | C | THR | A | 204 | 69.847 | 18.152 | 5.776 | 1.00 | 27.62 | C |
| ATOM | 1369 | O | THR | A | 204 | 68.680 | 17.948 | 5.397 | 1.00 | 27.26 | O |
| ATOM | 1370 | N | ARG | A | 205 | 70.483 | 17.391 | 6.670 | 1.00 | 27.57 | N |
| ATOM | 1371 | CA | ARG | A | 205 | 69.928 | 16.139 | 7.173 | 1.00 | 27.70 | C |
| ATOM | 1372 | CB | ARG | A | 205 | 70.881 | 15.491 | 8.193 | 1.00 | 28.59 | C |
| ATOM | 1373 | CG | ARG | A | 205 | 70.306 | 14.238 | 8.883 | 1.00 | 31.06 | C |
| ATOM | 1374 | CD | ARG | A | 205 | 71.326 | 13.172 | 9.299 | 1.00 | 34.02 | C |
| ATOM | 1375 | NE | ARG | A | 205 | 71.717 | 12.397 | 8.132 | 1.00 | 37.36 | N |
| ATOM | 1376 | CZ | ARG | A | 205 | 71.619 | 11.073 | 7.997 | 1.00 | 37.19 | C |
| ATOM | 1377 | NH1 | ARG | A | 205 | 71.156 | 10.302 | 8.970 | 1.00 | 36.97 | N |
| ATOM | 1378 | NH2 | ARG | A | 205 | 71.995 | 10.523 | 6.856 | 1.00 | 36.80 | N |
| ATOM | 1379 | C | ARG | A | 205 | 68.508 | 16.270 | 7.748 | 1.00 | 27.42 | C |
| ATOM | 1380 | O | ARG | A | 205 | 67.600 | 15.482 | 7.393 | 1.00 | 27.39 | O |
| ATOM | 1381 | N | VAL | A | 206 | 68.323 | 17.271 | 8.611 | 1.00 | 26.15 | N |
| ATOM | 1382 | CA | VAL | A | 206 | 67.088 | 17.452 | 9.368 | 1.00 | 24.94 | C |
| ATOM | 1383 | CB | VAL | A | 206 | 67.268 | 18.408 | 10.593 | 1.00 | 25.07 | C |
| ATOM | 1384 | CG1 | VAL | A | 206 | 68.149 | 17.763 | 11.651 | 1.00 | 24.65 | C |
| ATOM | 1385 | CG2 | VAL | A | 206 | 67.842 | 19.792 | 10.167 | 1.00 | 23.92 | C |
| ATOM | 1386 | C | VAL | A | 206 | 65.986 | 17.956 | 8.455 | 1.00 | 25.16 | C |
| ATOM | 1387 | O | VAL | A | 206 | 64.835 | 18.077 | 8.883 | 1.00 | 25.56 | O |
| ATOM | 1388 | N | TYR | A | 207 | 66.343 | 18.226 | 7.195 | 1.00 | 24.00 | N |
| ATOM | 1389 | CA | TYR | A | 207 | 65.363 | 18.533 | 6.169 | 1.00 | 23.56 | C |
| ATOM | 1390 | CB | TYR | A | 207 | 65.792 | 19.777 | 5.388 | 1.00 | 23.98 | C |
| ATOM | 1391 | CG | TYR | A | 207 | 65.472 | 21.091 | 6.067 | 1.00 | 23.10 | C |
| ATOM | 1392 | CD1 | TYR | A | 207 | 66.274 | 21.587 | 7.101 | 1.00 | 22.69 | C |
| ATOM | 1393 | CE1 | TYR | A | 207 | 65.980 | 22.819 | 7.722 | 1.00 | 24.39 | C |
| ATOM | 1394 | CZ | TYR | A | 207 | 64.884 | 23.551 | 7.277 | 1.00 | 27.17 | C |
| ATOM | 1395 | OH | TYR | A | 207 | 64.556 | 24.776 | 7.844 | 1.00 | 30.13 | O |
| ATOM | 1396 | CE2 | TYR | A | 207 | 64.080 | 23.064 | 6.243 | 1.00 | 25.83 | C |
| ATOM | 1397 | CD2 | TYR | A | 207 | 64.382 | 21.851 | 5.647 | 1.00 | 24.73 | C |
| ATOM | 1398 | C | TYR | A | 207 | 65.141 | 17.371 | 5.198 | 1.00 | 22.93 | C |
| ATOM | 1399 | O | TYR | A | 207 | 64.299 | 17.469 | 4.285 | 1.00 | 22.62 | O |
| ATOM | 1400 | N | SER | A | 208 | 65.906 | 16.292 | 5.382 | 1.00 | 21.96 | N |
| ATOM | 1401 | CA | SER | A | 208 | 65.849 | 15.131 | 4.487 | 1.00 | 21.81 | C |
| ATOM | 1402 | CB | SER | A | 208 | 67.203 | 14.408 | 4.432 | 1.00 | 22.13 | C |
| ATOM | 1403 | OG | SER | A | 208 | 67.426 | 13.650 | 5.611 | 1.00 | 23.96 | O |
| ATOM | 1404 | C | SER | A | 208 | 64.738 | 14.139 | 4.876 | 1.00 | 20.92 | C |
| ATOM | 1405 | O | SER | A | 208 | 64.427 | 13.970 | 6.062 | 1.00 | 20.62 | O |
| ATOM | 1406 | N | PRO | A | 209 | 64.177 | 13.466 | 3.873 | 1.00 | 19.97 | N |
| ATOM | 1407 | CA | PRO | A | 209 | 62.999 | 12.620 | 4.067 | 1.00 | 19.59 | C |
| ATOM | 1408 | CB | PRO | A | 209 | 62.467 | 12.452 | 2.639 | 1.00 | 19.55 | C |
| ATOM | 1409 | CG | PRO | A | 209 | 63.689 | 12.524 | 1.774 | 1.00 | 19.80 | C |
| ATOM | 1410 | CD | PRO | A | 209 | 64.644 | 13.447 | 2.470 | 1.00 | 19.85 | C |
| ATOM | 1411 | C | PRO | A | 209 | 63.380 | 11.269 | 4.690 | 1.00 | 19.33 | C |
| ATOM | 1412 | O | PRO | A | 209 | 64.554 | 10.867 | 4.623 | 1.00 | 19.13 | O |
| ATOM | 1413 | N | PRO | A | 210 | 62.415 | 10.592 | 5.304 | 1.00 | 18.92 | N |
| ATOM | 1414 | CA | PRO | A | 210 | 62.668 | 9.300 | 5.961 | 1.00 | 19.08 | C |
| ATOM | 1415 | CB | PRO | A | 210 | 61.301 | 8.921 | 6.557 | 1.00 | 18.83 | C |
| ATOM | 1416 | CG | PRO | A | 210 | 60.302 | 9.737 | 5.821 | 1.00 | 19.11 | C |
| ATOM | 1417 | CD | PRO | A | 210 | 61.006 | 11.018 | 5.435 | 1.00 | 18.89 | C |
| ATOM | 1418 | C | PRO | A | 210 | 63.164 | 8.203 | 5.012 | 1.00 | 19.74 | C |
| ATOM | 1419 | O | PRO | A | 210 | 63.892 | 7.324 | 5.476 | 1.00 | 19.91 | O |
| ATOM | 1420 | N | GLU | A | 211 | 62.796 | 8.256 | 3.732 | 1.00 | 19.73 | N |
| ATOM | 1421 | CA | GLU | A | 211 | 63.273 | 7.278 | 2.766 | 1.00 | 20.85 | C |
| ATOM | 1422 | CB | GLU | A | 211 | 62.461 | 7.323 | 1.451 | 1.00 | 20.53 | C |
| ATOM | 1423 | CG | GLU | A | 211 | 62.554 | 8.649 | 0.684 | 1.00 | 20.57 | C |
| ATOM | 1424 | CD | GLU | A | 211 | 61.446 | 9.651 | 1.014 | 1.00 | 20.44 | C |
| ATOM | 1425 | OE1 | GLU | A | 211 | 60.905 | 9.640 | 2.143 | 1.00 | 21.24 | O |
| ATOM | 1426 | OE2 | GLU | A | 211 | 61.122 | 10.474 | 0.132 | 1.00 | 19.92 | O |
| ATOM | 1427 | C | GLU | A | 211 | 64.782 | 7.446 | 2.532 | 1.00 | 21.48 | C |
| ATOM | 1428 | O | GLU | A | 211 | 65.491 | 6.465 | 2.284 | 1.00 | 21.77 | O |
| ATOM | 1429 | N | TRP | A | 212 | 65.269 | 8.683 | 2.624 | 1.00 | 21.85 | N |
| ATOM | 1430 | CA | TRP | A | 212 | 66.702 | 8.917 | 2.576 | 1.00 | 22.59 | C |
| ATOM | 1431 | CB | TRP | A | 212 | 67.059 | 10.402 | 2.439 | 1.00 | 21.76 | C |
| ATOM | 1432 | CG | TRP | A | 212 | 68.537 | 10.610 | 2.665 | 1.00 | 23.30 | C |
| ATOM | 1433 | CD1 | TRP | A | 212 | 69.128 | 11.209 | 3.745 | 1.00 | 23.54 | C |
| ATOM | 1434 | NE1 | TRP | A | 212 | 70.497 | 11.187 | 3.610 | 1.00 | 24.29 | N |
| ATOM | 1435 | CE2 | TRP | A | 212 | 70.828 | 10.556 | 2.441 | 1.00 | 23.65 | C |
| ATOM | 1436 | CD2 | TRP | A | 212 | 69.618 | 10.169 | 1.817 | 1.00 | 22.66 | C |
| ATOM | 1437 | CE3 | TRP | A | 212 | 69.684 | 9.497 | 0.589 | 1.00 | 22.34 | C |
| ATOM | 1438 | CZ3 | TRP | A | 212 | 70.944 | 9.227 | 0.028 | 1.00 | 23.22 | C |
| ATOM | 1439 | CH2 | TRP | A | 212 | 72.129 | 9.621 | 0.680 | 1.00 | 23.33 | C |
| ATOM | 1440 | CZ2 | TRP | A | 212 | 72.093 | 10.288 | 1.882 | 1.00 | 24.31 | C |
| ATOM | 1441 | C | TRP | A | 212 | 67.375 | 8.324 | 3.814 | 1.00 | 22.99 | C |
| ATOM | 1442 | O | TRP | A | 212 | 68.368 | 7.609 | 3.695 | 1.00 | 23.46 | O |
| ATOM | 1443 | N | ILE | A | 213 | 66.812 | 8.611 | 4.986 | 1.00 | 23.54 | N |
| ATOM | 1444 | CA | ILE | A | 213 | 67.375 | 8.166 | 6.265 | 1.00 | 24.57 | C |
| ATOM | 1445 | CB | ILE | A | 213 | 66.566 | 8.729 | 7.468 | 1.00 | 24.25 | C |
| ATOM | 1446 | CG1 | ILE | A | 213 | 66.550 | 10.265 | 7.469 | 1.00 | 24.58 | C |
| ATOM | 1447 | CD1 | ILE | A | 213 | 67.945 | 10.927 | 7.479 | 1.00 | 24.84 | C |
| ATOM | 1448 | CG2 | ILE | A | 213 | 67.143 | 8.217 | 8.788 | 1.00 | 24.24 | C |
| ATOM | 1449 | C | ILE | A | 213 | 67.480 | 6.646 | 6.350 | 1.00 | 25.27 | C |
| ATOM | 1450 | O | ILE | A | 213 | 68.523 | 6.113 | 6.741 | 1.00 | 25.42 | O |
| ATOM | 1451 | N | ARG | A | 214 | 66.409 | 5.963 | 5.955 | 1.00 | 26.19 | N |
| ATOM | 1452 | CA | ARG | A | 214 | 66.330 | 4.508 | 6.035 | 1.00 | 27.54 | C |
| ATOM | 1453 | CB | ARG | A | 214 | 64.873 | 4.052 | 6.126 | 1.00 | 27.97 | C |
| ATOM | 1454 | CG | ARG | A | 214 | 64.138 | 4.599 | 7.344 | 1.00 | 31.59 | C |
| ATOM | 1455 | CD | ARG | A | 214 | 62.621 | 4.436 | 7.296 | 1.00 | 37.14 | C |
| ATOM | 1456 | NE | ARG | A | 214 | 62.201 | 3.037 | 7.213 | 1.00 | 40.55 | N |
| ATOM | 1457 | CZ | ARG | A | 214 | 62.233 | 2.167 | 8.219 | 1.00 | 43.40 | C |
| ATOM | 1458 | NH1 | ARG | A | 214 | 62.672 | 2.525 | 9.423 | 1.00 | 44.08 | N |
| ATOM | 1459 | NH2 | ARG | A | 214 | 61.823 | 0.919 | 8.018 | 1.00 | 44.82 | N |
| ATOM | 1460 | C | ARG | A | 214 | 67.018 | 3.787 | 4.877 | 1.00 | 27.77 | C |
| ATOM | 1461 | O | ARG | A | 214 | 67.799 | 2.869 | 5.113 | 1.00 | 27.88 | O |
| ATOM | 1462 | N | TYR | A | 215 | 66.731 | 4.195 | 3.641 | 1.00 | 27.94 | N |
| ATOM | 1463 | CA | TYR | A | 215 | 67.151 | 3.428 | 2.460 | 1.00 | 28.52 | C |
| ATOM | 1464 | CB | TYR | A | 215 | 65.931 | 2.983 | 1.642 | 1.00 | 28.63 | C |
| ATOM | 1465 | CG | TYR | A | 215 | 64.788 | 2.478 | 2.486 | 1.00 | 30.63 | C |
| ATOM | 1466 | CD1 | TYR | A | 215 | 64.884 | 1.262 | 3.177 | 1.00 | 32.35 | C |
| ATOM | 1467 | CE1 | TYR | A | 215 | 63.832 | 0.800 | 3.965 | 1.00 | 33.17 | C |
| ATOM | 1468 | CZ | TYR | A | 215 | 62.674 | 1.560 | 4.063 | 1.00 | 34.20 | C |
| ATOM | 1469 | OH | TYR | A | 215 | 61.625 | 1.121 | 4.834 | 1.00 | 36.84 | O |
| ATOM | 1470 | CE2 | TYR | A | 215 | 62.556 | 2.764 | 3.390 | 1.00 | 33.32 | C |
| ATOM | 1471 | CD2 | TYR | A | 215 | 63.610 | 3.217 | 2.607 | 1.00 | 32.43 | C |
| ATOM | 1472 | C | TYR | A | 215 | 68.143 | 4.128 | 1.539 | 1.00 | 28.30 | C |
| ATOM | 1473 | O | TYR | A | 215 | 68.551 | 3.558 | 0.531 | 1.00 | 28.64 | O |
| ATOM | 1474 | N | HIS | A | 216 | 68.520 | 5.360 | 1.870 | 1.00 | 27.98 | N |
| ATOM | 1475 | CA | HIS | A | 216 | 69.431 | 6.133 | 1.027 | 1.00 | 27.92 | C |
| ATOM | 1476 | CB | HIS | A | 216 | 70.866 | 5.574 | 1.125 | 1.00 | 28.78 | C |
| ATOM | 1477 | CG | HIS | A | 216 | 71.628 | 6.080 | 2.315 | 1.00 | 32.47 | C |
| ATOM | 1478 | ND1 | HIS | A | 216 | 72.993 | 5.922 | 2.453 | 1.00 | 36.05 | N |
| ATOM | 1479 | CE1 | HIS | A | 216 | 73.386 | 6.479 | 3.587 | 1.00 | 37.27 | C |
| ATOM | 1480 | NE2 | HIS | A | 216 | 72.328 | 6.997 | 4.187 | 1.00 | 36.76 | N |
| ATOM | 1481 | CD2 | HIS | A | 216 | 71.217 | 6.761 | 3.413 | 1.00 | 35.02 | C |
| ATOM | 1482 | C | HIS | A | 216 | 68.936 | 6.268 | −0.435 | 1.00 | 26.74 | C |
| ATOM | 1483 | O | HIS | A | 216 | 69.728 | 6.295 | −1.383 | 1.00 | 26.90 | O |
| ATOM | 1484 | N | ARG | A | 217 | 67.616 | 6.360 | −0.592 | 1.00 | 25.13 | N |
| ATOM | 1485 | CA | ARG | A | 217 | 66.964 | 6.461 | −1.892 | 1.00 | 24.20 | C |
| ATOM | 1486 | CB | ARG | A | 217 | 66.380 | 5.106 | −2.341 | 1.00 | 24.35 | C |
| ATOM | 1487 | CG | ARG | A | 217 | 67.373 | 3.964 | −2.525 | 1.00 | 27.36 | C |
| ATOM | 1488 | CD | ARG | A | 217 | 66.718 | 2.584 | −2.668 | 1.00 | 31.36 | C |
| ATOM | 1489 | NE | ARG | A | 217 | 66.048 | 2.433 | −3.958 | 1.00 | 34.40 | N |
| ATOM | 1490 | CZ | ARG | A | 217 | 66.633 | 1.967 | −5.061 | 1.00 | 36.63 | C |
| ATOM | 1491 | NH1 | ARG | A | 217 | 67.909 | 1.595 | −5.043 | 1.00 | 36.66 | N |
| ATOM | 1492 | NH2 | ARG | A | 217 | 65.943 | 1.879 | −6.190 | 1.00 | 37.21 | N |
| ATOM | 1493 | C | ARG | A | 217 | 65.808 | 7.429 | −1.749 | 1.00 | 22.74 | C |
| ATOM | 1494 | O | ARG | A | 217 | 65.124 | 7.420 | −0.729 | 1.00 | 23.06 | O |
| ATOM | 1495 | N | TYR | A | 218 | 65.580 | 8.240 | −2.777 | 1.00 | 20.60 | N |
| ATOM | 1496 | CA | TYR | A | 218 | 64.445 | 9.141 | −2.824 | 1.00 | 18.74 | C |
| ATOM | 1497 | CB | TYR | A | 218 | 64.674 | 10.371 | −1.917 | 1.00 | 18.22 | C |
| ATOM | 1498 | CG | TYR | A | 218 | 65.867 | 11.216 | −2.299 | 1.00 | 16.94 | C |
| ATOM | 1499 | CD1 | TYR | A | 218 | 67.160 | 10.880 | −1.870 | 1.00 | 15.03 | C |
| ATOM | 1500 | CE1 | TYR | A | 218 | 68.265 | 11.679 | −2.220 | 1.00 | 14.80 | C |
| ATOM | 1501 | CZ | TYR | A | 218 | 68.064 | 12.802 | −3.020 | 1.00 | 15.81 | C |
| ATOM | 1502 | OH | TYR | A | 218 | 69.125 | 13.594 | −3.393 | 1.00 | 16.21 | O |
| ATOM | 1503 | CE2 | TYR | A | 218 | 66.790 | 13.145 | −3.453 | 1.00 | 15.34 | C |
| ATOM | 1504 | CD2 | TYR | A | 218 | 65.705 | 12.355 | −3.093 | 1.00 | 15.11 | C |
| ATOM | 1505 | C | TYR | A | 218 | 64.212 | 9.584 | −4.267 | 1.00 | 18.07 | C |
| ATOM | 1506 | O | TYR | A | 218 | 65.112 | 9.486 | −5.102 | 1.00 | 17.75 | O |
| ATOM | 1507 | N | HIS | A | 219 | 63.006 | 10.075 | −4.545 | 1.00 | 17.04 | N |
| ATOM | 1508 | CA | HIS | A | 219 | 62.721 | 10.757 | −5.811 | 1.00 | 16.74 | C |
| ATOM | 1509 | CB | HIS | A | 219 | 61.430 | 10.231 | −6.440 | 1.00 | 16.49 | C |
| ATOM | 1510 | CG | HIS | A | 219 | 61.547 | 8.819 | −6.917 | 1.00 | 18.35 | C |
| ATOM | 1511 | ND1 | HIS | A | 219 | 61.677 | 8.489 | −8.253 | 1.00 | 18.97 | N |
| ATOM | 1512 | CE1 | HIS | A | 219 | 61.791 | 7.179 | −8.368 | 1.00 | 17.84 | C |
| ATOM | 1513 | NE2 | HIS | A | 219 | 61.763 | 6.650 | −7.157 | 1.00 | 19.10 | N |
| ATOM | 1514 | CD2 | HIS | A | 219 | 61.621 | 7.653 | −6.230 | 1.00 | 16.82 | C |
| ATOM | 1515 | C | HIS | A | 219 | 62.651 | 12.255 | −5.551 | 1.00 | 16.15 | C |
| ATOM | 1516 | O | HIS | A | 219 | 62.346 | 12.681 | −4.438 | 1.00 | 15.76 | O |
| ATOM | 1517 | N | GLY | A | 220 | 62.938 | 13.048 | −6.576 | 1.00 | 16.23 | N |
| ATOM | 1518 | CA | GLY | A | 220 | 63.172 | 14.462 | −6.384 | 1.00 | 16.70 | C |
| ATOM | 1519 | C | GLY | A | 220 | 61.992 | 15.217 | −5.807 | 1.00 | 16.87 | C |
| ATOM | 1520 | O | GLY | A | 220 | 62.110 | 15.886 | −4.788 | 1.00 | 16.14 | O |
| ATOM | 1521 | N | ARG | A | 221 | 60.848 | 15.097 | −6.469 | 1.00 | 17.28 | N |
| ATOM | 1522 | CA | ARG | A | 221 | 59.691 | 15.923 | −6.150 | 1.00 | 17.64 | C |
| ATOM | 1523 | CB | ARG | A | 221 | 58.590 | 15.746 | −7.185 | 1.00 | 18.45 | C |
| ATOM | 1524 | CG | ARG | A | 221 | 59.002 | 16.190 | −8.578 | 1.00 | 23.24 | C |
| ATOM | 1525 | CD | ARG | A | 221 | 58.156 | 15.591 | −9.697 | 1.00 | 28.55 | C |
| ATOM | 1526 | NE | ARG | A | 221 | 58.705 | 15.933 | −11.013 | 1.00 | 32.56 | N |
| ATOM | 1527 | CZ | ARG | A | 221 | 59.512 | 15.147 | −11.720 | 1.00 | 35.42 | C |
| ATOM | 1528 | NH1 | ARG | A | 221 | 59.879 | 13.956 | −11.241 | 1.00 | 36.89 | N |
| ATOM | 1529 | NH2 | ARG | A | 221 | 59.943 | 15.539 | −12.920 | 1.00 | 35.53 | N |
| ATOM | 1530 | C | ARG | A | 221 | 59.155 | 15.652 | −4.764 | 1.00 | 16.82 | C |
| ATOM | 1531 | O | ARG | A | 221 | 58.922 | 16.596 | −4.015 | 1.00 | 16.95 | O |
| ATOM | 1532 | N | SER | A | 222 | 58.981 | 14.374 | −4.417 | 1.00 | 15.97 | N |
| ATOM | 1533 | CA | SER | A | 222 | 58.439 | 14.002 | −3.111 | 1.00 | 15.37 | C |
| ATOM | 1534 | CB | SER | A | 222 | 58.036 | 12.510 | −3.066 | 1.00 | 15.54 | C |
| ATOM | 1535 | OG | SER | A | 222 | 59.136 | 11.654 | −3.333 | 1.00 | 15.91 | O |
| ATOM | 1536 | C | SER | A | 222 | 59.396 | 14.347 | −1.971 | 1.00 | 14.74 | C |
| ATOM | 1537 | O | SER | A | 222 | 58.963 | 14.684 | −0.874 | 1.00 | 14.86 | O |
| ATOM | 1538 | N | ALA | A | 223 | 60.694 | 14.270 | −2.225 | 1.00 | 14.48 | N |
| ATOM | 1539 | CA | ALA | A | 223 | 61.686 | 14.738 | −1.253 | 1.00 | 14.36 | C |
| ATOM | 1540 | CB | ALA | A | 223 | 63.081 | 14.313 | −1.677 | 1.00 | 14.45 | C |
| ATOM | 1541 | C | ALA | A | 223 | 61.619 | 16.266 | −1.091 | 1.00 | 14.53 | C |
| ATOM | 1542 | O | ALA | A | 223 | 61.718 | 16.779 | 0.030 | 1.00 | 14.59 | O |
| ATOM | 1543 | N | ALA | A | 224 | 61.441 | 16.982 | −2.205 | 1.00 | 13.88 | N |
| ATOM | 1544 | CA | ALA | A | 224 | 61.310 | 18.444 | −2.169 | 1.00 | 13.96 | C |
| ATOM | 1545 | CB | ALA | A | 224 | 61.174 | 19.032 | −3.591 | 1.00 | 13.04 | C |
| ATOM | 1546 | C | ALA | A | 224 | 60.116 | 18.832 | −1.296 | 1.00 | 13.72 | C |
| ATOM | 1547 | O | ALA | A | 224 | 60.220 | 19.713 | −0.462 | 1.00 | 14.14 | O |
| ATOM | 1548 | N | VAL | A | 225 | 59.001 | 18.123 | −1.470 | 1.00 | 14.13 | N |
| ATOM | 1549 | CA | VAL | A | 225 | 57.776 | 18.363 | −0.704 | 1.00 | 13.34 | C |
| ATOM | 1550 | CB | VAL | A | 225 | 56.602 | 17.517 | −1.301 | 1.00 | 13.99 | C |
| ATOM | 1551 | CG1 | VAL | A | 225 | 55.370 | 17.457 | −0.352 | 1.00 | 11.55 | C |
| ATOM | 1552 | CG2 | VAL | A | 225 | 56.236 | 18.063 | −2.701 | 1.00 | 12.97 | C |
| ATOM | 1553 | C | VAL | A | 225 | 57.986 | 18.076 | 0.778 | 1.00 | 14.00 | C |
| ATOM | 1554 | O | VAL | A | 225 | 57.513 | 18.820 | 1.650 | 1.00 | 14.66 | O |
| ATOM | 1555 | N | TRP | A | 226 | 58.695 | 16.996 | 1.087 | 1.00 | 13.82 | N |
| ATOM | 1556 | CA | TRP | A | 226 | 59.037 | 16.751 | 2.481 | 1.00 | 14.22 | C |
| ATOM | 1557 | CB | TRP | A | 226 | 59.908 | 15.501 | 2.626 | 1.00 | 14.10 | C |
| ATOM | 1558 | CG | TRP | A | 226 | 60.362 | 15.305 | 4.045 | 1.00 | 14.21 | C |
| ATOM | 1559 | CD1 | TRP | A | 226 | 61.444 | 15.888 | 4.651 | 1.00 | 13.01 | C |
| ATOM | 1560 | NE1 | TRP | A | 226 | 61.516 | 15.488 | 5.961 | 1.00 | 13.97 | N |
| ATOM | 1561 | CE2 | TRP | A | 226 | 60.473 | 14.643 | 6.231 | 1.00 | 13.33 | C |
| ATOM | 1562 | CD2 | TRP | A | 226 | 59.723 | 14.510 | 5.046 | 1.00 | 14.66 | C |
| ATOM | 1563 | CE3 | TRP | A | 226 | 58.583 | 13.683 | 5.063 | 1.00 | 14.53 | C |
| ATOM | 1564 | CZ3 | TRP | A | 226 | 58.254 | 13.024 | 6.238 | 1.00 | 14.25 | C |
| ATOM | 1565 | CH2 | TRP | A | 226 | 59.020 | 13.173 | 7.395 | 1.00 | 14.22 | C |
| ATOM | 1566 | CZ2 | TRP | A | 226 | 60.132 | 13.981 | 7.415 | 1.00 | 15.55 | C |
| ATOM | 1567 | C | TRP | A | 226 | 59.763 | 17.976 | 3.062 | 1.00 | 13.86 | C |
| ATOM | 1568 | O | TRP | A | 226 | 59.406 | 18.468 | 4.136 | 1.00 | 14.16 | O |
| ATOM | 1569 | N | SER | A | 227 | 60.764 | 18.485 | 2.349 | 1.00 | 13.20 | N |
| ATOM | 1570 | CA | SER | A | 227 | 61.522 | 19.610 | 2.875 | 1.00 | 13.52 | C |
| ATOM | 1571 | CB | SER | A | 227 | 62.793 | 19.886 | 2.043 | 1.00 | 13.28 | C |
| ATOM | 1572 | OG | SER | A | 227 | 62.448 | 20.474 | 0.803 | 1.00 | 12.96 | O |
| ATOM | 1573 | C | SER | A | 227 | 60.634 | 20.845 | 2.967 | 1.00 | 13.72 | C |
| ATOM | 1574 | O | SER | A | 227 | 60.848 | 21.702 | 3.816 | 1.00 | 13.60 | O |
| ATOM | 1575 | N | LEU | A | 228 | 59.614 | 20.917 | 2.110 | 1.00 | 13.17 | N |
| ATOM | 1576 | CA | LEU | A | 228 | 58.673 | 22.028 | 2.171 | 1.00 | 13.14 | C |
| ATOM | 1577 | CB | LEU | A | 228 | 57.807 | 22.105 | 0.891 | 1.00 | 12.32 | C |
| ATOM | 1578 | CG | LEU | A | 228 | 58.606 | 22.646 | −0.297 | 1.00 | 11.69 | C |
| ATOM | 1579 | CD1 | LEU | A | 228 | 57.931 | 22.321 | −1.659 | 1.00 | 13.88 | C |
| ATOM | 1580 | CD2 | LEU | A | 228 | 58.893 | 24.135 | −0.171 | 1.00 | 10.58 | C |
| ATOM | 1581 | C | LEU | A | 228 | 57.801 | 21.968 | 3.427 | 1.00 | 12.15 | C |
| ATOM | 1582 | O | LEU | A | 228 | 57.489 | 22.990 | 4.020 | 1.00 | 12.32 | O |
| ATOM | 1583 | N | GLY | A | 229 | 57.405 | 20.766 | 3.811 | 1.00 | 12.02 | N |
| ATOM | 1584 | CA | GLY | A | 229 | 56.693 | 20.556 | 5.056 | 1.00 | 12.19 | C |
| ATOM | 1585 | C | GLY | A | 229 | 57.512 | 20.973 | 6.281 | 1.00 | 12.64 | C |
| ATOM | 1586 | O | GLY | A | 229 | 56.968 | 21.560 | 7.224 | 1.00 | 12.42 | O |
| ATOM | 1587 | N | ILE | A | 230 | 58.811 | 20.662 | 6.269 | 1.00 | 13.07 | N |
| ATOM | 1588 | CA | ILE | A | 230 | 59.718 | 21.050 | 7.357 | 1.00 | 13.99 | C |
| ATOM | 1589 | CB | ILE | A | 230 | 61.145 | 20.421 | 7.133 | 1.00 | 13.90 | C |
| ATOM | 1590 | CG1 | ILE | A | 230 | 61.067 | 18.894 | 7.053 | 1.00 | 13.25 | C |
| ATOM | 1591 | CD1 | ILE | A | 230 | 60.622 | 18.243 | 8.368 | 1.00 | 11.51 | C |
| ATOM | 1592 | CG2 | ILE | A | 230 | 62.118 | 20.815 | 8.272 | 1.00 | 14.90 | C |
| ATOM | 1593 | C | ILE | A | 230 | 59.785 | 22.587 | 7.409 | 1.00 | 14.39 | C |
| ATOM | 1594 | O | ILE | A | 230 | 59.686 | 23.211 | 8.488 | 1.00 | 14.11 | O |
| ATOM | 1595 | N | LEU | A | 231 | 59.915 | 23.182 | 6.222 | 1.00 | 14.04 | N |
| ATOM | 1596 | CA | LEU | A | 231 | 59.961 | 24.620 | 6.083 | 1.00 | 14.10 | C |
| ATOM | 1597 | CB | LEU | A | 231 | 60.197 | 24.995 | 4.609 | 1.00 | 14.16 | C |
| ATOM | 1598 | CG | LEU | A | 231 | 60.121 | 26.508 | 4.330 | 1.00 | 15.24 | C |
| ATOM | 1599 | CD1 | LEU | A | 231 | 61.292 | 27.224 | 4.967 | 1.00 | 15.13 | C |
| ATOM | 1600 | CD2 | LEU | A | 231 | 60.075 | 26.778 | 2.838 | 1.00 | 15.78 | C |
| ATOM | 1601 | C | LEU | A | 231 | 58.688 | 25.299 | 6.615 | 1.00 | 14.01 | C |
| ATOM | 1602 | O | LEU | A | 231 | 58.775 | 26.270 | 7.382 | 1.00 | 13.50 | O |
| ATOM | 1603 | N | LEU | A | 232 | 57.515 | 24.796 | 6.217 | 1.00 | 13.22 | N |
| ATOM | 1604 | CA | LEU | A | 232 | 56.256 | 25.394 | 6.644 | 1.00 | 13.16 | C |
| ATOM | 1605 | CB | LEU | A | 232 | 55.031 | 24.750 | 5.949 | 1.00 | 13.56 | C |
| ATOM | 1606 | CG | LEU | A | 232 | 53.653 | 25.362 | 6.282 | 1.00 | 12.22 | C |
| ATOM | 1607 | CD1 | LEU | A | 232 | 53.627 | 26.902 | 6.159 | 1.00 | 13.83 | C |
| ATOM | 1608 | CD2 | LEU | A | 232 | 52.521 | 24.721 | 5.419 | 1.00 | 11.85 | C |
| ATOM | 1609 | C | LEU | A | 232 | 56.112 | 25.294 | 8.164 | 1.00 | 13.79 | C |
| ATOM | 1610 | O | LEU | A | 232 | 55.723 | 26.269 | 8.817 | 1.00 | 14.65 | O |
| ATOM | 1611 | N | TYR | A | 233 | 56.445 | 24.133 | 8.723 | 1.00 | 13.34 | N |
| ATOM | 1612 | CA | TYR | A | 233 | 56.407 | 23.940 | 10.175 | 1.00 | 13.68 | C |
| ATOM | 1613 | CB | TYR | A | 233 | 56.870 | 22.524 | 10.551 | 1.00 | 12.73 | C |
| ATOM | 1614 | CG | TYR | A | 233 | 56.786 | 22.263 | 12.044 | 1.00 | 14.51 | C |
| ATOM | 1615 | CD1 | TYR | A | 233 | 57.791 | 22.713 | 12.908 | 1.00 | 14.02 | C |
| ATOM | 1616 | CE1 | TYR | A | 233 | 57.728 | 22.487 | 14.266 | 1.00 | 13.74 | C |
| ATOM | 1617 | CZ | TYR | A | 233 | 56.647 | 21.796 | 14.798 | 1.00 | 15.95 | C |
| ATOM | 1618 | OH | TYR | A | 233 | 56.590 | 21.586 | 16.169 | 1.00 | 17.38 | O |
| ATOM | 1619 | CE2 | TYR | A | 233 | 55.630 | 21.352 | 13.978 | 1.00 | 15.69 | C |
| ATOM | 1620 | CD2 | TYR | A | 233 | 55.705 | 21.588 | 12.594 | 1.00 | 14.19 | C |
| ATOM | 1621 | C | TYR | A | 233 | 57.296 | 24.989 | 10.855 | 1.00 | 14.14 | C |
| ATOM | 1622 | O | TYR | A | 233 | 56.893 | 25.639 | 11.837 | 1.00 | 14.22 | O |
| ATOM | 1623 | N | ASP | A | 234 | 58.497 | 25.162 | 10.304 | 1.00 | 14.64 | N |
| ATOM | 1624 | CA | ASP | A | 234 | 59.462 | 26.128 | 10.820 | 1.00 | 14.70 | C |
| ATOM | 1625 | CB | ASP | A | 234 | 60.741 | 26.074 | 9.986 | 1.00 | 15.23 | C |
| ATOM | 1626 | CG | ASP | A | 234 | 61.806 | 27.031 | 10.482 | 1.00 | 17.95 | C |
| ATOM | 1627 | OD1 | ASP | A | 234 | 62.134 | 27.038 | 11.693 | 1.00 | 17.66 | O |
| ATOM | 1628 | OD2 | ASP | A | 234 | 62.372 | 27.815 | 9.707 | 1.00 | 23.64 | O |
| ATOM | 1629 | C | ASP | A | 234 | 58.902 | 27.550 | 10.842 | 1.00 | 15.14 | C |
| ATOM | 1630 | O | ASP | A | 234 | 59.130 | 28.304 | 11.808 | 1.00 | 14.44 | O |
| ATOM | 1631 | N | MET | A | 235 | 58.177 | 27.921 | 9.782 | 1.00 | 14.52 | N |
| ATOM | 1632 | CA | MET | A | 235 | 57.585 | 29.248 | 9.679 | 1.00 | 15.77 | C |
| ATOM | 1633 | CB | MET | A | 235 | 56.946 | 29.472 | 8.300 | 1.00 | 15.95 | C |
| ATOM | 1634 | CG | MET | A | 235 | 57.955 | 29.567 | 7.157 | 1.00 | 19.29 | C |
| ATOM | 1635 | SD | MET | A | 235 | 57.147 | 30.105 | 5.616 | 1.00 | 23.96 | S |
| ATOM | 1636 | CE | MET | A | 235 | 56.577 | 28.752 | 5.093 | 1.00 | 24.70 | C |
| ATOM | 1637 | C | MET | A | 235 | 56.535 | 29.503 | 10.756 | 1.00 | 15.44 | C |
| ATOM | 1638 | O | MET | A | 235 | 56.551 | 30.545 | 11.395 | 1.00 | 15.28 | O |
| ATOM | 1639 | N | VAL | A | 236 | 55.622 | 28.557 | 10.944 | 1.00 | 15.79 | N |
| ATOM | 1640 | CA | VAL | A | 236 | 54.480 | 28.780 | 11.845 | 1.00 | 16.11 | C |
| ATOM | 1641 | CB | VAL | A | 236 | 53.169 | 28.062 | 11.349 | 1.00 | 16.48 | C |
| ATOM | 1642 | CG1 | VAL | A | 236 | 52.709 | 28.622 | 9.995 | 1.00 | 15.52 | C |
| ATOM | 1643 | CG2 | VAL | A | 236 | 53.327 | 26.521 | 11.277 | 1.00 | 14.68 | C |
| ATOM | 1644 | C | VAL | A | 236 | 54.808 | 28.422 | 13.295 | 1.00 | 17.29 | C |
| ATOM | 1645 | O | VAL | A | 236 | 54.084 | 28.833 | 14.220 | 1.00 | 17.67 | O |
| ATOM | 1646 | N | CYS | A | 237 | 55.901 | 27.673 | 13.503 | 1.00 | 17.50 | N |
| ATOM | 1647 | CA | CYS | A | 237 | 56.276 | 27.261 | 14.863 | 1.00 | 18.98 | C |
| ATOM | 1648 | CB | CYS | A | 237 | 56.400 | 25.735 | 14.986 | 1.00 | 18.51 | C |
| ATOM | 1649 | SG | CYS | A | 237 | 54.825 | 24.891 | 14.842 | 1.00 | 22.03 | S |
| ATOM | 1650 | C | CYS | A | 237 | 57.548 | 27.914 | 15.366 | 1.00 | 18.98 | C |
| ATOM | 1651 | O | CYS | A | 237 | 57.788 | 27.936 | 16.562 | 1.00 | 18.75 | O |
| ATOM | 1652 | N | GLY | A | 238 | 58.359 | 28.443 | 14.452 | 1.00 | 19.38 | N |
| ATOM | 1653 | CA | GLY | A | 238 | 59.584 | 29.125 | 14.835 | 1.00 | 20.34 | C |
| ATOM | 1654 | C | GLY | A | 238 | 60.776 | 28.206 | 14.966 | 1.00 | 21.28 | C |
| ATOM | 1655 | O | GLY | A | 238 | 61.871 | 28.677 | 15.269 | 1.00 | 21.63 | O |
| ATOM | 1656 | N | ASP | A | 239 | 60.572 | 26.906 | 14.743 | 1.00 | 21.89 | N |
| ATOM | 1657 | CA | ASP | A | 239 | 61.662 | 25.904 | 14.741 | 1.00 | 23.14 | C |
| ATOM | 1658 | CB | ASP | A | 239 | 62.038 | 25.466 | 16.161 | 1.00 | 24.31 | C |
| ATOM | 1659 | CG | ASP | A | 239 | 63.557 | 25.367 | 16.363 | 1.00 | 28.93 | C |
| ATOM | 1660 | OD1 | ASP | A | 239 | 64.268 | 24.729 | 15.530 | 1.00 | 31.83 | O |
| ATOM | 1661 | OD2 | ASP | A | 239 | 64.126 | 25.919 | 17.333 | 1.00 | 34.08 | O |
| ATOM | 1662 | C | ASP | A | 239 | 61.271 | 24.671 | 13.918 | 1.00 | 22.10 | C |
| ATOM | 1663 | O | ASP | A | 239 | 60.110 | 24.508 | 13.582 | 1.00 | 22.05 | O |
| ATOM | 1664 | N | ILE | A | 240 | 62.241 | 23.823 | 13.590 | 1.00 | 21.41 | N |
| ATOM | 1665 | CA | ILE | A | 240 | 61.995 | 22.616 | 12.803 | 1.00 | 21.20 | C |
| ATOM | 1666 | CB | ILE | A | 240 | 63.299 | 22.130 | 12.119 | 1.00 | 21.27 | C |
| ATOM | 1667 | CG1 | ILE | A | 240 | 64.418 | 21.934 | 13.162 | 1.00 | 22.95 | C |
| ATOM | 1668 | CD1 | ILE | A | 240 | 65.727 | 21.359 | 12.604 | 1.00 | 24.55 | C |
| ATOM | 1669 | CG2 | ILE | A | 240 | 63.711 | 23.113 | 11.020 | 1.00 | 22.14 | C |
| ATOM | 1670 | C | ILE | A | 240 | 61.390 | 21.516 | 13.687 | 1.00 | 21.05 | C |
| ATOM | 1671 | O | ILE | A | 240 | 61.628 | 21.507 | 14.896 | 1.00 | 20.43 | O |
| ATOM | 1672 | N | PRO | A | 241 | 60.596 | 20.610 | 13.112 | 1.00 | 21.20 | N |
| ATOM | 1673 | CA | PRO | A | 241 | 59.885 | 19.609 | 13.924 | 1.00 | 22.14 | C |
| ATOM | 1674 | CB | PRO | A | 241 | 58.818 | 19.070 | 12.967 | 1.00 | 22.34 | C |
| ATOM | 1675 | CG | PRO | A | 241 | 59.418 | 19.243 | 11.581 | 1.00 | 20.54 | C |
| ATOM | 1676 | CD | PRO | A | 241 | 60.303 | 20.461 | 11.670 | 1.00 | 21.11 | C |
| ATOM | 1677 | C | PRO | A | 241 | 60.762 | 18.466 | 14.413 | 1.00 | 23.21 | C |
| ATOM | 1678 | O | PRO | A | 241 | 60.432 | 17.885 | 15.443 | 1.00 | 23.48 | O |
| ATOM | 1679 | N | PHE | A | 242 | 61.843 | 18.143 | 13.699 | 1.00 | 24.34 | N |
| ATOM | 1680 | CA | PHE | A | 242 | 62.625 | 16.949 | 13.999 | 1.00 | 25.31 | C |
| ATOM | 1681 | CB | PHE | A | 242 | 62.503 | 15.894 | 12.881 | 1.00 | 24.74 | C |
| ATOM | 1682 | CG | PHE | A | 242 | 61.097 | 15.596 | 12.440 | 1.00 | 22.55 | C |
| ATOM | 1683 | CD1 | PHE | A | 242 | 60.115 | 15.212 | 13.354 | 1.00 | 21.74 | C |
| ATOM | 1684 | CE1 | PHE | A | 242 | 58.812 | 14.916 | 12.923 | 1.00 | 21.18 | C |
| ATOM | 1685 | CZ | PHE | A | 242 | 58.489 | 15.011 | 11.556 | 1.00 | 21.06 | C |
| ATOM | 1686 | CE2 | PHE | A | 242 | 59.467 | 15.392 | 10.642 | 1.00 | 20.29 | C |
| ATOM | 1687 | CD2 | PHE | A | 242 | 60.763 | 15.672 | 11.088 | 1.00 | 21.16 | C |
| ATOM | 1688 | C | PHE | A | 242 | 64.099 | 17.286 | 14.169 | 1.00 | 27.27 | C |
| ATOM | 1689 | O | PHE | A | 242 | 64.671 | 18.038 | 13.370 | 1.00 | 27.35 | O |
| ATOM | 1690 | N | GLU | A | 243 | 64.716 | 16.692 | 15.186 | 1.00 | 29.13 | N |
| ATOM | 1691 | CA | GLU | A | 243 | 66.149 | 16.849 | 15.428 | 1.00 | 31.65 | C |
| ATOM | 1692 | CB | GLU | A | 243 | 66.404 | 17.361 | 16.849 | 1.00 | 32.51 | C |
| ATOM | 1693 | CG | GLU | A | 243 | 65.779 | 18.723 | 17.153 | 1.00 | 37.83 | C |
| ATOM | 1694 | CD | GLU | A | 243 | 66.521 | 19.895 | 16.505 | 1.00 | 43.98 | C |
| ATOM | 1695 | OE1 | GLU | A | 243 | 66.675 | 19.903 | 15.260 | 1.00 | 46.61 | O |
| ATOM | 1696 | OE2 | GLU | A | 243 | 66.941 | 20.824 | 17.241 | 1.00 | 46.61 | O |
| ATOM | 1697 | C | GLU | A | 243 | 66.951 | 15.568 | 15.187 | 1.00 | 31.62 | C |
| ATOM | 1698 | O | GLU | A | 243 | 68.096 | 15.630 | 14.759 | 1.00 | 32.81 | O |
| ATOM | 1699 | N | HIS | A | 244 | 66.361 | 14.409 | 15.454 | 1.00 | 31.55 | N |
| ATOM | 1700 | CA | HIS | A | 244 | 67.089 | 13.146 | 15.307 | 1.00 | 31.42 | C |
| ATOM | 1701 | CB | HIS | A | 244 | 67.187 | 12.423 | 16.650 | 1.00 | 32.01 | C |
| ATOM | 1702 | CG | HIS | A | 244 | 67.774 | 13.265 | 17.738 | 1.00 | 34.56 | C |
| ATOM | 1703 | ND1 | HIS | A | 244 | 67.014 | 13.790 | 18.763 | 1.00 | 36.67 | N |
| ATOM | 1704 | CE1 | HIS | A | 244 | 67.791 | 14.502 | 19.561 | 1.00 | 37.86 | C |
| ATOM | 1705 | NE2 | HIS | A | 244 | 69.026 | 14.462 | 19.087 | 1.00 | 37.93 | N |
| ATOM | 1706 | CD2 | HIS | A | 244 | 69.041 | 13.697 | 17.945 | 1.00 | 36.34 | C |
| ATOM | 1707 | C | HIS | A | 244 | 66.482 | 12.235 | 14.243 | 1.00 | 30.37 | C |
| ATOM | 1708 | O | HIS | A | 244 | 65.279 | 12.327 | 13.941 | 1.00 | 29.56 | O |
| ATOM | 1709 | N | ASP | A | 245 | 67.326 | 11.360 | 13.689 | 1.00 | 29.19 | N |
| ATOM | 1710 | CA | ASP | A | 245 | 66.909 | 10.373 | 12.691 | 1.00 | 28.54 | C |
| ATOM | 1711 | CB | ASP | A | 245 | 68.005 | 9.315 | 12.473 | 1.00 | 28.34 | C |
| ATOM | 1712 | CG | ASP | A | 245 | 69.208 | 9.853 | 11.726 | 1.00 | 28.71 | C |
| ATOM | 1713 | OD1 | ASP | A | 245 | 69.183 | 11.016 | 11.252 | 1.00 | 28.60 | O |
| ATOM | 1714 | OD2 | ASP | A | 245 | 70.242 | 9.174 | 11.572 | 1.00 | 30.07 | O |
| ATOM | 1715 | C | ASP | A | 245 | 65.624 | 9.670 | 13.103 | 1.00 | 28.04 | C |
| ATOM | 1716 | O | ASP | A | 245 | 64.724 | 9.485 | 12.284 | 1.00 | 27.65 | O |
| ATOM | 1717 | N | GLU | A | 246 | 65.566 | 9.292 | 14.381 | 1.00 | 27.40 | N |
| ATOM | 1718 | CA | GLU | A | 246 | 64.451 | 8.563 | 14.978 | 1.00 | 27.37 | C |
| ATOM | 1719 | CB | GLU | A | 246 | 64.743 | 8.286 | 16.468 | 1.00 | 28.11 | C |
| ATOM | 1720 | CG | GLU | A | 246 | 65.942 | 7.369 | 16.736 | 1.00 | 32.49 | C |
| ATOM | 1721 | CD | GLU | A | 246 | 67.302 | 8.072 | 16.650 | 1.00 | 37.16 | C |
| ATOM | 1722 | OE1 | GLU | A | 246 | 67.413 | 9.255 | 17.037 | 1.00 | 39.44 | O |
| ATOM | 1723 | OE2 | GLU | A | 246 | 68.276 | 7.434 | 16.191 | 1.00 | 40.05 | O |
| ATOM | 1724 | C | GLU | A | 246 | 63.128 | 9.318 | 14.844 | 1.00 | 26.19 | C |
| ATOM | 1725 | O | GLU | A | 246 | 62.087 | 8.720 | 14.570 | 1.00 | 25.74 | O |
| ATOM | 1726 | N | GLU | A | 247 | 63.178 | 10.630 | 15.054 | 1.00 | 24.84 | N |
| ATOM | 1727 | CA | GLU | A | 247 | 61.997 | 11.473 | 14.925 | 1.00 | 24.64 | C |
| ATOM | 1728 | CB | GLU | A | 247 | 62.228 | 12.861 | 15.550 | 1.00 | 24.76 | C |
| ATOM | 1729 | CG | GLU | A | 247 | 62.600 | 12.823 | 17.029 | 1.00 | 27.07 | C |
| ATOM | 1730 | CD | GLU | A | 247 | 63.106 | 14.164 | 17.539 | 1.00 | 31.82 | C |
| ATOM | 1731 | OE1 | GLU | A | 247 | 63.956 | 14.804 | 16.873 | 1.00 | 31.23 | O |
| ATOM | 1732 | OE2 | GLU | A | 247 | 62.653 | 14.582 | 18.623 | 1.00 | 35.62 | O |
| ATOM | 1733 | C | GLU | A | 247 | 61.573 | 11.592 | 13.458 | 1.00 | 23.43 | C |
| ATOM | 1734 | O | GLU | A | 247 | 60.388 | 11.491 | 13.151 | 1.00 | 22.84 | O |
| ATOM | 1735 | N | ILE | A | 248 | 62.546 | 11.782 | 12.563 | 1.00 | 22.82 | N |
| ATOM | 1736 | CA | ILE | A | 248 | 62.269 | 11.827 | 11.119 | 1.00 | 22.09 | C |
| ATOM | 1737 | CB | ILE | A | 248 | 63.555 | 12.106 | 10.284 | 1.00 | 22.33 | C |
| ATOM | 1738 | CG1 | ILE | A | 248 | 64.103 | 13.506 | 10.594 | 1.00 | 21.24 | C |
| ATOM | 1739 | CD1 | ILE | A | 248 | 65.557 | 13.692 | 10.191 | 1.00 | 23.01 | C |
| ATOM | 1740 | CG2 | ILE | A | 248 | 63.273 | 11.965 | 8.767 | 1.00 | 21.11 | C |
| ATOM | 1741 | C | ILE | A | 248 | 61.567 | 10.547 | 10.665 | 1.00 | 22.18 | C |
| ATOM | 1742 | O | ILE | A | 248 | 60.512 | 10.608 | 10.038 | 1.00 | 21.20 | O |
| ATOM | 1743 | N | ILE | A | 249 | 62.144 | 9.396 | 11.016 | 1.00 | 22.55 | N |
| ATOM | 1744 | CA | ILE | A | 249 | 61.595 | 8.083 | 10.646 | 1.00 | 23.21 | C |
| ATOM | 1745 | CB | ILE | A | 249 | 62.539 | 6.943 | 11.136 | 1.00 | 23.49 | C |
| ATOM | 1746 | CG1 | ILE | A | 249 | 63.856 | 6.947 | 10.348 | 1.00 | 24.43 | C |
| ATOM | 1747 | CD1 | ILE | A | 249 | 64.971 | 6.114 | 11.041 | 1.00 | 26.84 | C |
| ATOM | 1748 | CG2 | ILE | A | 249 | 61.853 | 5.562 | 11.051 | 1.00 | 24.03 | C |
| ATOM | 1749 | C | ILE | A | 249 | 60.175 | 7.875 | 11.200 | 1.00 | 23.18 | C |
| ATOM | 1750 | O | ILE | A | 249 | 59.312 | 7.314 | 10.520 | 1.00 | 22.99 | O |
| ATOM | 1751 | N | ARG | A | 250 | 59.943 | 8.318 | 12.435 | 1.00 | 23.13 | N |
| ATOM | 1752 | CA | ARG | A | 250 | 58.614 | 8.204 | 13.044 | 1.00 | 23.60 | C |
| ATOM | 1753 | CB | ARG | A | 250 | 58.679 | 8.421 | 14.562 | 1.00 | 23.56 | C |
| ATOM | 1754 | CG | ARG | A | 250 | 57.356 | 8.162 | 15.290 | 1.00 | 24.55 | C |
| ATOM | 1755 | CD | ARG | A | 250 | 57.504 | 7.770 | 16.760 | 1.00 | 24.93 | C |
| ATOM | 1756 | NE | ARG | A | 250 | 56.208 | 7.638 | 17.430 | 1.00 | 25.45 | N |
| ATOM | 1757 | CZ | ARG | A | 250 | 55.636 | 8.594 | 18.168 | 1.00 | 25.98 | C |
| ATOM | 1758 | NH1 | ARG | A | 250 | 56.234 | 9.770 | 18.349 | 1.00 | 24.37 | N |
| ATOM | 1759 | NH2 | ARG | A | 250 | 54.459 | 8.373 | 18.733 | 1.00 | 26.41 | N |
| ATOM | 1760 | C | ARG | A | 250 | 57.621 | 9.159 | 12.375 | 1.00 | 23.56 | C |
| ATOM | 1761 | O | ARG | A | 250 | 56.468 | 8.802 | 12.165 | 1.00 | 23.53 | O |
| ATOM | 1762 | N | GLY | A | 251 | 58.089 | 10.357 | 12.022 | 1.00 | 23.63 | N |
| ATOM | 1763 | CA | GLY | A | 251 | 57.282 | 11.334 | 11.314 | 1.00 | 24.56 | C |
| ATOM | 1764 | C | GLY | A | 251 | 56.082 | 11.864 | 12.096 | 1.00 | 25.30 | C |
| ATOM | 1765 | O | GLY | A | 251 | 55.074 | 12.248 | 11.496 | 1.00 | 25.76 | O |
| ATOM | 1766 | N | GLN | A | 252 | 56.177 | 11.877 | 13.423 | 1.00 | 25.26 | N |
| ATOM | 1767 | CA | GLN | A | 252 | 55.082 | 12.373 | 14.263 | 1.00 | 25.84 | C |
| ATOM | 1768 | CB | GLN | A | 252 | 55.005 | 11.603 | 15.593 | 1.00 | 26.06 | C |
| ATOM | 1769 | CG | GLN | A | 252 | 53.796 | 11.937 | 16.488 | 1.00 | 29.12 | C |
| ATOM | 1770 | CD | GLN | A | 252 | 52.439 | 11.649 | 15.837 | 1.00 | 32.13 | C |
| ATOM | 1771 | OE1 | GLN | A | 252 | 51.537 | 12.506 | 15.854 | 1.00 | 31.35 | O |
| ATOM | 1772 | NE2 | GLN | A | 252 | 52.292 | 10.448 | 15.264 | 1.00 | 32.42 | N |
| ATOM | 1773 | C | GLN | A | 252 | 55.271 | 13.863 | 14.504 | 1.00 | 25.11 | C |
| ATOM | 1774 | O | GLN | A | 252 | 56.310 | 14.303 | 15.008 | 1.00 | 24.98 | O |
| ATOM | 1775 | N | VAL | A | 253 | 54.265 | 14.634 | 14.123 | 1.00 | 24.33 | N |
| ATOM | 1776 | CA | VAL | A | 253 | 54.351 | 16.085 | 14.196 | 1.00 | 24.36 | C |
| ATOM | 1777 | CB | VAL | A | 253 | 53.751 | 16.750 | 12.922 | 1.00 | 24.24 | C |
| ATOM | 1778 | CG1 | VAL | A | 253 | 53.971 | 18.240 | 12.948 | 1.00 | 24.73 | C |
| ATOM | 1779 | CG2 | VAL | A | 253 | 54.356 | 16.124 | 11.647 | 1.00 | 24.87 | C |
| ATOM | 1780 | C | VAL | A | 253 | 53.601 | 16.576 | 15.431 | 1.00 | 23.69 | C |
| ATOM | 1781 | O | VAL | A | 253 | 52.427 | 16.275 | 15.602 | 1.00 | 23.22 | O |
| ATOM | 1782 | N | PHE | A | 254 | 54.297 | 17.321 | 16.278 | 1.00 | 23.14 | N |
| ATOM | 1783 | CA | PHE | A | 254 | 53.683 | 17.993 | 17.403 | 1.00 | 23.19 | C |
| ATOM | 1784 | CB | PHE | A | 254 | 54.295 | 17.481 | 18.702 | 1.00 | 22.64 | C |
| ATOM | 1785 | CG | APHE | A | 254 | 53.868 | 18.247 | 19.912 | 0.70 | 21.91 | C |
| ATOM | 1786 | CG | BPHE | A | 254 | 53.915 | 16.067 | 18.997 | 0.30 | 22.47 | C |
| ATOM | 1787 | CD1 | APHE | A | 254 | 52.711 | 17.897 | 20.596 | 0.70 | 20.57 | C |
| ATOM | 1788 | CD1 | BPHE | A | 254 | 54.877 | 15.073 | 19.055 | 0.30 | 21.39 | C |
| ATOM | 1789 | CE1 | APHE | A | 254 | 52.308 | 18.608 | 21.724 | 0.70 | 18.82 | C |
| ATOM | 1790 | CE1 | BPHE | A | 254 | 54.517 | 13.772 | 19.304 | 0.30 | 20.97 | C |
| ATOM | 1791 | CZ | APHE | A | 254 | 53.054 | 19.677 | 22.163 | 0.70 | 20.33 | C |
| ATOM | 1792 | CZ | BPHE | A | 254 | 53.180 | 13.445 | 19.476 | 0.30 | 21.52 | C |
| ATOM | 1793 | CE2 | APHE | A | 254 | 54.214 | 20.045 | 21.486 | 0.70 | 20.58 | C |
| ATOM | 1794 | CE2 | BPHE | A | 254 | 52.207 | 14.421 | 19.400 | 0.30 | 21.67 | C |
| ATOM | 1795 | CD2 | APHE | A | 254 | 54.615 | 19.333 | 20.369 | 0.70 | 21.56 | C |
| ATOM | 1796 | CD2 | BPHE | A | 254 | 52.574 | 15.720 | 19.157 | 0.30 | 21.61 | C |
| ATOM | 1797 | C | PHE | A | 254 | 53.789 | 19.507 | 17.295 | 1.00 | 23.65 | C |
| ATOM | 1798 | O | PHE | A | 254 | 54.876 | 20.055 | 17.059 | 1.00 | 23.03 | O |
| ATOM | 1799 | N | PHE | A | 255 | 52.652 | 20.173 | 17.475 | 1.00 | 24.25 | N |
| ATOM | 1800 | CA | PHE | A | 255 | 52.600 | 21.636 | 17.448 | 1.00 | 24.57 | C |
| ATOM | 1801 | CB | PHE | A | 255 | 51.367 | 22.117 | 16.705 | 1.00 | 24.07 | C |
| ATOM | 1802 | CG | PHE | A | 255 | 51.421 | 21.819 | 15.250 | 1.00 | 22.93 | C |
| ATOM | 1803 | CD1 | PHE | A | 255 | 51.972 | 22.743 | 14.368 | 1.00 | 20.88 | C |
| ATOM | 1804 | CE1 | PHE | A | 255 | 52.048 | 22.466 | 13.018 | 1.00 | 18.91 | C |
| ATOM | 1805 | CZ | PHE | A | 255 | 51.585 | 21.254 | 12.540 | 1.00 | 20.61 | C |
| ATOM | 1806 | CE2 | PHE | A | 255 | 51.047 | 20.307 | 13.426 | 1.00 | 19.19 | C |
| ATOM | 1807 | CD2 | PHE | A | 255 | 50.974 | 20.595 | 14.762 | 1.00 | 19.54 | C |
| ATOM | 1808 | C | PHE | A | 255 | 52.668 | 22.239 | 18.830 | 1.00 | 25.43 | C |
| ATOM | 1809 | O | PHE | A | 255 | 51.839 | 21.958 | 19.691 | 1.00 | 25.30 | O |
| ATOM | 1810 | N | ARG | A | 256 | 53.701 | 23.057 | 19.015 | 1.00 | 26.64 | N |
| ATOM | 1811 | CA | ARG | A | 256 | 54.026 | 23.713 | 20.274 | 1.00 | 27.50 | C |
| ATOM | 1812 | CB | ARG | A | 256 | 55.556 | 23.791 | 20.412 | 1.00 | 28.35 | C |
| ATOM | 1813 | CG | ARG | A | 256 | 56.282 | 24.268 | 19.116 | 1.00 | 31.19 | C |
| ATOM | 1814 | CD | ARG | A | 256 | 57.825 | 24.203 | 19.164 | 1.00 | 35.66 | C |
| ATOM | 1815 | NE | ARG | A | 256 | 58.346 | 23.171 | 18.257 | 1.00 | 38.13 | N |
| ATOM | 1816 | CZ | ARG | A | 256 | 59.580 | 22.660 | 18.298 | 1.00 | 40.12 | C |
| ATOM | 1817 | NH1 | ARG | A | 256 | 60.466 | 23.080 | 19.204 | 1.00 | 40.47 | N |
| ATOM | 1818 | NH2 | ARG | A | 256 | 59.930 | 21.716 | 17.431 | 1.00 | 38.85 | N |
| ATOM | 1819 | C | ARG | A | 256 | 53.444 | 25.122 | 20.255 | 1.00 | 27.08 | C |
| ATOM | 1820 | O | ARG | A | 256 | 53.389 | 25.807 | 21.279 | 1.00 | 27.90 | O |
| ATOM | 1821 | N | GLN | A | 257 | 53.023 | 25.546 | 19.068 | 1.00 | 26.07 | N |
| ATOM | 1822 | CA | GLN | A | 257 | 52.369 | 26.834 | 18.871 | 1.00 | 25.36 | C |
| ATOM | 1823 | CB | GLN | A | 257 | 53.126 | 27.643 | 17.803 | 1.00 | 25.81 | C |
| ATOM | 1824 | CG | GLN | A | 257 | 54.514 | 28.095 | 18.215 | 1.00 | 29.91 | C |
| ATOM | 1825 | CD | GLN | A | 257 | 54.493 | 29.381 | 19.019 | 1.00 | 35.63 | C |
| ATOM | 1826 | OE1 | GLN | A | 257 | 53.596 | 30.222 | 18.846 | 1.00 | 37.93 | O |
| ATOM | 1827 | NE2 | GLN | A | 257 | 55.480 | 29.545 | 19.901 | 1.00 | 37.58 | N |
| ATOM | 1828 | C | GLN | A | 257 | 50.931 | 26.611 | 18.399 | 1.00 | 23.15 | C |
| ATOM | 1829 | O | GLN | A | 257 | 50.618 | 25.574 | 17.821 | 1.00 | 22.57 | O |
| ATOM | 1830 | N | ARG | A | 258 | 50.072 | 27.593 | 18.633 | 1.00 | 21.04 | N |
| ATOM | 1831 | CA | ARG | A | 258 | 48.726 | 27.571 | 18.079 | 1.00 | 19.58 | C |
| ATOM | 1832 | CB | ARG | A | 258 | 47.862 | 28.674 | 18.683 | 1.00 | 19.69 | C |
| ATOM | 1833 | CG | ARG | A | 258 | 46.355 | 28.438 | 18.509 | 1.00 | 21.79 | C |
| ATOM | 1834 | CD | ARG | A | 258 | 45.834 | 28.809 | 17.134 | 1.00 | 24.81 | C |
| ATOM | 1835 | NE | ARG | A | 258 | 44.538 | 28.195 | 16.847 | 1.00 | 26.49 | N |
| ATOM | 1836 | CZ | ARG | A | 258 | 43.844 | 28.395 | 15.725 | 1.00 | 27.04 | C |
| ATOM | 1837 | NH1 | ARG | A | 258 | 44.316 | 29.200 | 14.757 | 1.00 | 27.33 | N |
| ATOM | 1838 | NH2 | ARG | A | 258 | 42.677 | 27.789 | 15.570 | 1.00 | 25.64 | N |
| ATOM | 1839 | C | ARG | A | 258 | 48.811 | 27.738 | 16.564 | 1.00 | 18.78 | C |
| ATOM | 1840 | O | ARG | A | 258 | 49.282 | 28.759 | 16.074 | 1.00 | 18.29 | O |
| ATOM | 1841 | N | VAL | A | 259 | 48.367 | 26.716 | 15.843 | 1.00 | 17.49 | N |
| ATOM | 1842 | CA | VAL | A | 259 | 48.404 | 26.682 | 14.389 | 1.00 | 17.02 | C |
| ATOM | 1843 | CB | VAL | A | 259 | 49.533 | 25.733 | 13.879 | 1.00 | 16.57 | C |
| ATOM | 1844 | CG1 | VAL | A | 259 | 49.472 | 25.544 | 12.361 | 1.00 | 15.19 | C |
| ATOM | 1845 | CG2 | VAL | A | 259 | 50.929 | 26.259 | 14.310 | 1.00 | 17.98 | C |
| ATOM | 1846 | C | VAL | A | 259 | 47.043 | 26.171 | 13.920 | 1.00 | 16.99 | C |
| ATOM | 1847 | O | VAL | A | 259 | 46.541 | 25.190 | 14.460 | 1.00 | 16.43 | O |
| ATOM | 1848 | N | SER | A | 260 | 46.451 | 26.843 | 12.930 | 1.00 | 17.02 | N |
| ATOM | 1849 | CA | SER | A | 260 | 45.141 | 26.451 | 12.398 | 1.00 | 17.31 | C |
| ATOM | 1850 | CB | SER | A | 260 | 44.687 | 27.398 | 11.273 | 1.00 | 17.29 | C |
| ATOM | 1851 | OG | SER | A | 260 | 45.399 | 27.137 | 10.073 | 1.00 | 17.50 | O |
| ATOM | 1852 | C | SER | A | 260 | 45.145 | 25.005 | 11.916 | 1.00 | 17.62 | C |
| ATOM | 1853 | O | SER | A | 260 | 46.182 | 24.484 | 11.518 | 1.00 | 17.40 | O |
| ATOM | 1854 | N | SER | A | 261 | 43.974 | 24.367 | 11.957 | 1.00 | 17.98 | N |
| ATOM | 1855 | CA | SER | A | 261 | 43.820 | 22.972 | 11.559 | 1.00 | 18.43 | C |
| ATOM | 1856 | CB | SER | A | 261 | 42.398 | 22.499 | 11.855 | 1.00 | 18.46 | C |
| ATOM | 1857 | OG | SER | A | 261 | 42.169 | 22.473 | 13.254 | 1.00 | 19.59 | O |
| ATOM | 1858 | C | SER | A | 261 | 44.118 | 22.748 | 10.082 | 1.00 | 18.64 | C |
| ATOM | 1859 | O | SER | A | 261 | 44.630 | 21.694 | 9.701 | 1.00 | 18.31 | O |
| ATOM | 1860 | N | GLU | A | 262 | 43.780 | 23.729 | 9.256 | 1.00 | 19.27 | N |
| ATOM | 1861 | CA | GLU | A | 262 | 44.102 | 23.659 | 7.829 | 1.00 | 20.49 | C |
| ATOM | 1862 | CB | GLU | A | 262 | 43.461 | 24.807 | 7.058 | 1.00 | 21.45 | C |
| ATOM | 1863 | CG | GLU | A | 262 | 42.033 | 24.525 | 6.627 | 1.00 | 27.18 | C |
| ATOM | 1864 | CD | GLU | A | 262 | 41.304 | 25.782 | 6.184 | 1.00 | 35.25 | C |
| ATOM | 1865 | OE1 | GLU | A | 262 | 41.928 | 26.645 | 5.498 | 1.00 | 38.82 | O |
| ATOM | 1866 | OE2 | GLU | A | 262 | 40.101 | 25.915 | 6.522 | 1.00 | 39.29 | O |
| ATOM | 1867 | C | GLU | A | 262 | 45.615 | 23.663 | 7.614 | 1.00 | 19.31 | C |
| ATOM | 1868 | O | GLU | A | 262 | 46.131 | 22.851 | 6.853 | 1.00 | 18.98 | O |
| ATOM | 1869 | N | CYS | A | 263 | 46.318 | 24.561 | 8.297 | 1.00 | 18.97 | N |
| ATOM | 1870 | CA | CYS | A | 263 | 47.785 | 24.596 | 8.196 | 1.00 | 18.66 | C |
| ATOM | 1871 | CB | CYS | A | 263 | 48.359 | 25.805 | 8.937 | 1.00 | 18.56 | C |
| ATOM | 1872 | SG | CYS | A | 263 | 50.133 | 26.031 | 8.731 | 1.00 | 18.69 | S |
| ATOM | 1873 | C | CYS | A | 263 | 48.385 | 23.275 | 8.703 | 1.00 | 18.45 | C |
| ATOM | 1874 | O | CYS | A | 263 | 49.223 | 22.664 | 8.024 | 1.00 | 18.06 | O |
| ATOM | 1875 | N | GLN | A | 264 | 47.932 | 22.827 | 9.873 | 1.00 | 18.01 | N |
| ATOM | 1876 | CA | GLN | A | 264 | 48.389 | 21.553 | 10.434 | 1.00 | 18.32 | C |
| ATOM | 1877 | CB | GLN | A | 264 | 47.650 | 21.210 | 11.748 | 1.00 | 17.97 | C |
| ATOM | 1878 | CG | GLN | A | 264 | 48.085 | 22.034 | 12.955 | 1.00 | 18.25 | C |
| ATOM | 1879 | CD | GLN | A | 264 | 47.598 | 21.447 | 14.282 | 1.00 | 20.77 | C |
| ATOM | 1880 | OE1 | GLN | A | 264 | 47.359 | 20.240 | 14.382 | 1.00 | 19.45 | O |
| ATOM | 1881 | NE2 | GLN | A | 264 | 47.464 | 22.299 | 15.304 | 1.00 | 19.10 | N |
| ATOM | 1882 | C | GLN | A | 264 | 48.191 | 20.419 | 9.424 | 1.00 | 18.18 | C |
| ATOM | 1883 | O | GLN | A | 264 | 49.068 | 19.581 | 9.252 | 1.00 | 18.03 | O |
| ATOM | 1884 | N | HIS | A | 265 | 47.033 | 20.405 | 8.768 | 1.00 | 18.36 | N |
| ATOM | 1885 | CA | HIS | A | 265 | 46.712 | 19.366 | 7.805 | 1.00 | 19.15 | C |
| ATOM | 1886 | CB | HIS | A | 265 | 45.269 | 19.505 | 7.310 | 1.00 | 19.80 | C |
| ATOM | 1887 | CG | HIS | A | 265 | 44.890 | 18.474 | 6.295 | 1.00 | 23.71 | C |
| ATOM | 1888 | ND1 | HIS | A | 265 | 45.147 | 18.627 | 4.948 | 1.00 | 27.52 | N |
| ATOM | 1889 | CE1 | HIS | A | 265 | 44.712 | 17.562 | 4.294 | 1.00 | 28.98 | C |
| ATOM | 1890 | NE2 | HIS | A | 265 | 44.190 | 16.720 | 5.170 | 1.00 | 29.95 | N |
| ATOM | 1891 | CD2 | HIS | A | 265 | 44.294 | 17.264 | 6.430 | 1.00 | 27.50 | C |
| ATOM | 1892 | C | HIS | A | 265 | 47.701 | 19.383 | 6.624 | 1.00 | 18.47 | C |
| ATOM | 1893 | O | HIS | A | 265 | 48.219 | 18.340 | 6.236 | 1.00 | 17.33 | O |
| ATOM | 1894 | N | LEU | A | 266 | 47.973 | 20.577 | 6.089 | 1.00 | 17.86 | N |
| ATOM | 1895 | CA | LEU | A | 266 | 48.891 | 20.717 | 4.968 | 1.00 | 17.70 | C |
| ATOM | 1896 | CB | LEU | A | 266 | 48.925 | 22.167 | 4.440 | 1.00 | 17.79 | C |
| ATOM | 1897 | CG | LEU | A | 266 | 49.889 | 22.490 | 3.277 | 1.00 | 16.98 | C |
| ATOM | 1898 | CD1 | LEU | A | 266 | 49.700 | 21.533 | 2.080 | 1.00 | 16.25 | C |
| ATOM | 1899 | CD2 | LEU | A | 266 | 49.731 | 23.941 | 2.832 | 1.00 | 16.33 | C |
| ATOM | 1900 | C | LEU | A | 266 | 50.282 | 20.225 | 5.372 | 1.00 | 17.61 | C |
| ATOM | 1901 | O | LEU | A | 266 | 50.906 | 19.443 | 4.642 | 1.00 | 17.30 | O |
| ATOM | 1902 | N | ILE | A | 267 | 50.741 | 20.639 | 6.555 | 1.00 | 17.19 | N |
| ATOM | 1903 | CA | ILE | A | 267 | 52.072 | 20.263 | 7.023 | 1.00 | 16.80 | C |
| ATOM | 1904 | CB | ILE | A | 267 | 52.425 | 20.934 | 8.385 | 1.00 | 16.67 | C |
| ATOM | 1905 | CG1 | ILE | A | 267 | 52.702 | 22.433 | 8.196 | 1.00 | 15.36 | C |
| ATOM | 1906 | CD1 | ILE | A | 267 | 52.656 | 23.273 | 9.494 | 1.00 | 14.12 | C |
| ATOM | 1907 | CG2 | ILE | A | 267 | 53.626 | 20.245 | 9.024 | 1.00 | 15.66 | C |
| ATOM | 1908 | C | ILE | A | 267 | 52.173 | 18.753 | 7.137 | 1.00 | 17.49 | C |
| ATOM | 1909 | O | ILE | A | 267 | 53.119 | 18.156 | 6.618 | 1.00 | 16.97 | O |
| ATOM | 1910 | N | ARG | A | 268 | 51.178 | 18.140 | 7.783 | 1.00 | 17.52 | N |
| ATOM | 1911 | CA | ARG | A | 268 | 51.165 | 16.692 | 7.997 | 1.00 | 18.02 | C |
| ATOM | 1912 | CB | ARG | A | 268 | 49.990 | 16.302 | 8.907 | 1.00 | 18.56 | C |
| ATOM | 1913 | CG | ARG | A | 268 | 50.240 | 16.587 | 10.386 | 1.00 | 20.63 | C |
| ATOM | 1914 | CD | ARG | A | 268 | 49.234 | 15.899 | 11.331 | 1.00 | 25.57 | C |
| ATOM | 1915 | NE | ARG | A | 268 | 48.912 | 16.743 | 12.487 | 1.00 | 29.85 | N |
| ATOM | 1916 | CZ | ARG | A | 268 | 49.629 | 16.737 | 13.585 | 1.00 | 31.14 | C |
| ATOM | 1917 | NH1 | ARG | A | 268 | 50.663 | 15.929 | 13.648 | 1.00 | 34.34 | N |
| ATOM | 1918 | NH2 | ARG | A | 268 | 49.331 | 17.507 | 14.615 | 1.00 | 30.34 | N |
| ATOM | 1919 | C | ARG | A | 268 | 51.104 | 15.910 | 6.668 | 1.00 | 17.59 | C |
| ATOM | 1920 | O | ARG | A | 268 | 51.676 | 14.833 | 6.544 | 1.00 | 16.65 | O |
| ATOM | 1921 | N | TRP | A | 269 | 50.397 | 16.470 | 5.693 | 1.00 | 17.56 | N |
| ATOM | 1922 | CA | TRP | A | 269 | 50.336 | 15.913 | 4.341 | 1.00 | 18.04 | C |
| ATOM | 1923 | CB | TRP | A | 269 | 49.340 | 16.717 | 3.490 | 1.00 | 18.77 | C |
| ATOM | 1924 | CG | TRP | A | 269 | 48.810 | 15.979 | 2.265 | 1.00 | 21.08 | C |
| ATOM | 1925 | CD1 | TRP | A | 269 | 49.030 | 14.662 | 1.914 | 1.00 | 22.56 | C |
| ATOM | 1926 | NE1 | TRP | A | 269 | 48.387 | 14.372 | 0.730 | 1.00 | 24.35 | N |
| ATOM | 1927 | CE2 | TRP | A | 269 | 47.716 | 15.491 | 0.301 | 1.00 | 23.34 | C |
| ATOM | 1928 | CD2 | TRP | A | 269 | 47.957 | 16.522 | 1.248 | 1.00 | 22.70 | C |
| ATOM | 1929 | CE3 | TRP | A | 269 | 47.377 | 17.781 | 1.033 | 1.00 | 23.10 | C |
| ATOM | 1930 | CZ3 | TRP | A | 269 | 46.576 | 17.970 | −0.102 | 1.00 | 24.93 | C |
| ATOM | 1931 | CH2 | TRP | A | 269 | 46.351 | 16.922 | −1.014 | 1.00 | 24.84 | C |
| ATOM | 1932 | CZ2 | TRP | A | 269 | 46.911 | 15.679 | −0.829 | 1.00 | 23.75 | C |
| ATOM | 1933 | C | TRP | A | 269 | 51.711 | 15.906 | 3.667 | 1.00 | 17.27 | C |
| ATOM | 1934 | O | TRP | A | 269 | 52.137 | 14.870 | 3.149 | 1.00 | 16.99 | O |
| ATOM | 1935 | N | CYS | A | 270 | 52.400 | 17.056 | 3.687 | 1.00 | 16.34 | N |
| ATOM | 1936 | CA | CYS | A | 270 | 53.759 | 17.173 | 3.134 | 1.00 | 16.21 | C |
| ATOM | 1937 | CB | CYS | A | 270 | 54.294 | 18.607 | 3.275 | 1.00 | 15.97 | C |
| ATOM | 1938 | SG | CYS | A | 270 | 53.427 | 19.842 | 2.287 | 1.00 | 16.89 | S |
| ATOM | 1939 | C | CYS | A | 270 | 54.742 | 16.241 | 3.824 | 1.00 | 16.04 | C |
| ATOM | 1940 | O | CYS | A | 270 | 55.711 | 15.774 | 3.195 | 1.00 | 15.33 | O |
| ATOM | 1941 | N | LEU | A | 271 | 54.488 | 15.978 | 5.112 | 1.00 | 15.59 | N |
| ATOM | 1942 | CA | LEU | A | 271 | 55.357 | 15.124 | 5.907 | 1.00 | 16.36 | C |
| ATOM | 1943 | CB | LEU | A | 271 | 55.574 | 15.727 | 7.304 | 1.00 | 15.90 | C |
| ATOM | 1944 | CG | LEU | A | 271 | 56.248 | 17.116 | 7.361 | 1.00 | 16.31 | C |
| ATOM | 1945 | CD1 | LEU | A | 271 | 56.473 | 17.592 | 8.793 | 1.00 | 13.91 | C |
| ATOM | 1946 | CD2 | LEU | A | 271 | 57.590 | 17.113 | 6.570 | 1.00 | 14.64 | C |
| ATOM | 1947 | C | LEU | A | 271 | 54.861 | 13.667 | 6.010 | 1.00 | 16.92 | C |
| ATOM | 1948 | O | LEU | A | 271 | 55.190 | 12.969 | 6.976 | 1.00 | 17.11 | O |
| ATOM | 1949 | N | ALA | A | 272 | 54.085 | 13.217 | 5.021 | 1.00 | 17.22 | N |
| ATOM | 1950 | CA | ALA | A | 272 | 53.627 | 11.819 | 4.971 | 1.00 | 18.16 | C |
| ATOM | 1951 | CB | ALA | A | 272 | 52.691 | 11.581 | 3.798 | 1.00 | 18.05 | C |
| ATOM | 1952 | C | ALA | A | 272 | 54.839 | 10.921 | 4.852 | 1.00 | 18.59 | C |
| ATOM | 1953 | O | ALA | A | 272 | 55.768 | 11.216 | 4.083 | 1.00 | 18.17 | O |
| ATOM | 1954 | N | LEU | A | 273 | 54.835 | 9.835 | 5.621 | 1.00 | 19.19 | N |
| ATOM | 1955 | CA | LEU | A | 273 | 55.953 | 8.894 | 5.630 | 1.00 | 19.94 | C |
| ATOM | 1956 | CB | LEU | A | 273 | 55.754 | 7.832 | 6.728 | 1.00 | 20.56 | C |
| ATOM | 1957 | CG | LEU | A | 273 | 56.079 | 8.298 | 8.162 | 1.00 | 21.18 | C |
| ATOM | 1958 | CD1 | LEU | A | 273 | 55.894 | 7.176 | 9.193 | 1.00 | 21.79 | C |
| ATOM | 1959 | CD2 | LEU | A | 273 | 57.491 | 8.889 | 8.262 | 1.00 | 20.34 | C |
| ATOM | 1960 | C | LEU | A | 273 | 56.178 | 8.254 | 4.258 | 1.00 | 20.58 | C |
| ATOM | 1961 | O | LEU | A | 273 | 57.322 | 8.138 | 3.800 | 1.00 | 20.82 | O |
| ATOM | 1962 | N | ARG | A | 274 | 55.090 | 7.849 | 3.605 | 1.00 | 20.78 | N |
| ATOM | 1963 | CA | ARG | A | 274 | 55.164 | 7.292 | 2.259 | 1.00 | 21.86 | C |
| ATOM | 1964 | CB | ARG | A | 274 | 53.968 | 6.373 | 1.955 | 1.00 | 22.23 | C |
| ATOM | 1965 | CG | ARG | A | 274 | 53.714 | 5.266 | 2.975 | 1.00 | 27.91 | C |
| ATOM | 1966 | CD | ARG | A | 274 | 52.588 | 4.263 | 2.576 | 1.00 | 35.10 | C |
| ATOM | 1967 | NE | ARG | A | 274 | 52.637 | 3.917 | 1.150 | 1.00 | 40.44 | N |
| ATOM | 1968 | CZ | ARG | A | 274 | 51.914 | 2.962 | 0.564 | 1.00 | 44.04 | C |
| ATOM | 1969 | NH1 | ARG | A | 274 | 51.061 | 2.223 | 1.275 | 1.00 | 45.20 | N |
| ATOM | 1970 | NH2 | ARG | A | 274 | 52.047 | 2.741 | −0.742 | 1.00 | 44.81 | N |
| ATOM | 1971 | C | ARG | A | 274 | 55.226 | 8.418 | 1.227 | 1.00 | 20.85 | C |
| ATOM | 1972 | O | ARG | A | 274 | 54.312 | 9.249 | 1.157 | 1.00 | 20.77 | O |
| ATOM | 1973 | N | PRO | A | 275 | 56.297 | 8.452 | 0.435 | 1.00 | 20.06 | N |
| ATOM | 1974 | CA | PRO | A | 275 | 56.479 | 9.502 | −0.576 | 1.00 | 20.15 | C |
| ATOM | 1975 | CB | PRO | A | 275 | 57.684 | 9.007 | −1.384 | 1.00 | 19.41 | C |
| ATOM | 1976 | CG | PRO | A | 275 | 58.448 | 8.169 | −0.409 | 1.00 | 19.87 | C |
| ATOM | 1977 | CD | PRO | A | 275 | 57.432 | 7.509 | 0.469 | 1.00 | 20.03 | C |
| ATOM | 1978 | C | PRO | A | 275 | 55.245 | 9.709 | −1.474 | 1.00 | 20.50 | C |
| ATOM | 1979 | O | PRO | A | 275 | 54.842 | 10.860 | −1.692 | 1.00 | 20.44 | O |
| ATOM | 1980 | N | SER | A | 276 | 54.650 | 8.618 | −1.966 | 1.00 | 20.58 | N |
| ATOM | 1981 | CA | SER | A | 276 | 53.454 | 8.693 | −2.811 | 1.00 | 20.56 | C |
| ATOM | 1982 | CB | SER | A | 276 | 53.146 | 7.323 | −3.430 | 1.00 | 20.69 | C |
| ATOM | 1983 | OG | SER | A | 276 | 52.516 | 6.487 | −2.479 | 1.00 | 22.25 | O |
| ATOM | 1984 | C | SER | A | 276 | 52.219 | 9.234 | −2.067 | 1.00 | 20.21 | C |
| ATOM | 1985 | O | SER | A | 276 | 51.232 | 9.612 | −2.697 | 1.00 | 20.43 | O |
| ATOM | 1986 | N | ASP | A | 277 | 52.264 | 9.266 | −0.737 | 1.00 | 19.88 | N |
| ATOM | 1987 | CA | ASP | A | 277 | 51.173 | 9.875 | 0.027 | 1.00 | 19.72 | C |
| ATOM | 1988 | CB | ASP | A | 277 | 51.093 | 9.311 | 1.443 | 1.00 | 19.57 | C |
| ATOM | 1989 | CG | ASP | A | 277 | 50.404 | 7.945 | 1.501 | 1.00 | 21.58 | C |
| ATOM | 1990 | OD1 | ASP | A | 277 | 49.751 | 7.540 | 0.504 | 1.00 | 20.40 | O |
| ATOM | 1991 | OD2 | ASP | A | 277 | 50.470 | 7.222 | 2.522 | 1.00 | 21.84 | O |
| ATOM | 1992 | C | ASP | A | 277 | 51.266 | 11.407 | 0.085 | 1.00 | 19.39 | C |
| ATOM | 1993 | O | ASP | A | 277 | 50.295 | 12.068 | 0.483 | 1.00 | 20.39 | O |
| ATOM | 1994 | N | ARG | A | 278 | 52.413 | 11.962 | −0.308 | 1.00 | 18.14 | N |
| ATOM | 1995 | CA | ARG | A | 278 | 52.633 | 13.408 | −0.252 | 1.00 | 17.60 | C |
| ATOM | 1996 | CB | ARG | A | 278 | 54.137 | 13.740 | −0.277 | 1.00 | 17.19 | C |
| ATOM | 1997 | CG | ARG | A | 278 | 54.859 | 13.330 | 1.009 | 1.00 | 16.29 | C |
| ATOM | 1998 | CD | ARG | A | 278 | 56.388 | 13.456 | 0.995 | 1.00 | 15.61 | C |
| ATOM | 1999 | NE | ARG | A | 278 | 56.954 | 12.458 | 1.908 | 1.00 | 15.33 | N |
| ATOM | 2000 | CZ | ARG | A | 278 | 58.152 | 11.885 | 1.769 | 1.00 | 15.76 | C |
| ATOM | 2001 | NH1 | ARG | A | 278 | 58.965 | 12.239 | 0.770 | 1.00 | 13.75 | N |
| ATOM | 2002 | NH2 | ARG | A | 278 | 58.541 | 10.966 | 2.649 | 1.00 | 13.90 | N |
| ATOM | 2003 | C | ARG | A | 278 | 51.908 | 14.104 | −1.391 | 1.00 | 17.58 | C |
| ATOM | 2004 | O | ARG | A | 278 | 51.716 | 13.506 | −2.466 | 1.00 | 17.77 | O |
| ATOM | 2005 | N | PRO | A | 279 | 51.508 | 15.357 | −1.166 | 1.00 | 16.97 | N |
| ATOM | 2006 | CA | PRO | A | 279 | 50.820 | 16.142 | −2.192 | 1.00 | 16.86 | C |
| ATOM | 2007 | CB | PRO | A | 279 | 50.364 | 17.387 | −1.415 | 1.00 | 17.33 | C |
| ATOM | 2008 | CG | PRO | A | 279 | 51.426 | 17.533 | −0.313 | 1.00 | 16.46 | C |
| ATOM | 2009 | CD | PRO | A | 279 | 51.685 | 16.125 | 0.088 | 1.00 | 16.42 | C |
| ATOM | 2010 | C | PRO | A | 279 | 51.768 | 16.573 | −3.290 | 1.00 | 17.64 | C |
| ATOM | 2011 | O | PRO | A | 279 | 52.967 | 16.757 | −3.021 | 1.00 | 17.91 | O |
| ATOM | 2012 | N | THR | A | 280 | 51.245 | 16.735 | −4.507 | 1.00 | 17.35 | N |
| ATOM | 2013 | CA | THR | A | 280 | 51.998 | 17.353 | −5.593 | 1.00 | 17.56 | C |
| ATOM | 2014 | CB | THR | A | 280 | 51.284 | 17.108 | −6.937 | 1.00 | 17.70 | C |
| ATOM | 2015 | OG1 | THR | A | 280 | 49.989 | 17.716 | −6.885 | 1.00 | 18.13 | O |
| ATOM | 2016 | CG2 | THR | A | 280 | 50.976 | 15.600 | −7.152 | 1.00 | 18.27 | C |
| ATOM | 2017 | C | THR | A | 280 | 52.048 | 18.864 | −5.326 | 1.00 | 18.09 | C |
| ATOM | 2018 | O | THR | A | 280 | 51.342 | 19.358 | −4.427 | 1.00 | 18.12 | O |
| ATOM | 2019 | N | PHE | A | 281 | 52.838 | 19.600 | −6.113 | 1.00 | 18.50 | N |
| ATOM | 2020 | CA | PHE | A | 281 | 52.870 | 21.066 | −6.000 | 1.00 | 19.56 | C |
| ATOM | 2021 | CB | PHE | A | 281 | 53.863 | 21.702 | −6.994 | 1.00 | 19.97 | C |
| ATOM | 2022 | CG | PHE | A | 281 | 55.322 | 21.369 | −6.721 | 1.00 | 22.52 | C |
| ATOM | 2023 | CD1 | PHE | A | 281 | 55.834 | 21.383 | −5.433 | 1.00 | 25.27 | C |
| ATOM | 2024 | CE1 | PHE | A | 281 | 57.198 | 21.070 | −5.177 | 1.00 | 26.96 | C |
| ATOM | 2025 | CZ | PHE | A | 281 | 58.040 | 20.748 | −6.235 | 1.00 | 29.27 | C |
| ATOM | 2026 | CE2 | PHE | A | 281 | 57.535 | 20.748 | −7.553 | 1.00 | 28.17 | C |
| ATOM | 2027 | CD2 | PHE | A | 281 | 56.183 | 21.061 | −7.781 | 1.00 | 26.44 | C |
| ATOM | 2028 | C | PHE | A | 281 | 51.474 | 21.656 | −6.211 | 1.00 | 19.44 | C |
| ATOM | 2029 | O | PHE | A | 281 | 51.064 | 22.559 | −5.481 | 1.00 | 19.75 | O |
| ATOM | 2030 | N | GLU | A | 282 | 50.742 | 21.119 | −7.188 | 1.00 | 18.94 | N |
| ATOM | 2031 | CA | GLU | A | 282 | 49.396 | 21.592 | −7.492 | 1.00 | 19.12 | C |
| ATOM | 2032 | CB | GLU | A | 282 | 48.851 | 20.940 | −8.787 | 1.00 | 19.45 | C |
| ATOM | 2033 | CG | GLU | A | 282 | 47.360 | 21.133 | −9.034 | 1.00 | 21.47 | C |
| ATOM | 2034 | CD | GLU | A | 282 | 46.874 | 20.578 | −10.387 | 1.00 | 26.22 | C |
| ATOM | 2035 | OE1 | GLU | A | 282 | 47.449 | 19.584 | −10.886 | 1.00 | 26.51 | O |
| ATOM | 2036 | OE2 | GLU | A | 282 | 45.901 | 21.136 | −10.951 | 1.00 | 25.95 | O |
| ATOM | 2037 | C | GLU | A | 282 | 48.454 | 21.369 | −6.307 | 1.00 | 18.61 | C |
| ATOM | 2038 | O | GLU | A | 282 | 47.648 | 22.248 | −5.986 | 1.00 | 18.93 | O |
| ATOM | 2039 | N | GLU | A | 283 | 48.558 | 20.219 | −5.640 | 1.00 | 17.63 | N |
| ATOM | 2040 | CA | GLU | A | 283 | 47.693 | 19.956 | −4.485 | 1.00 | 17.55 | C |
| ATOM | 2041 | CB | GLU | A | 283 | 47.744 | 18.489 | −4.076 | 1.00 | 17.90 | C |
| ATOM | 2042 | CG | GLU | A | 283 | 46.951 | 17.556 | −4.989 | 1.00 | 18.94 | C |
| ATOM | 2043 | CD | GLU | A | 283 | 47.298 | 16.099 | −4.752 | 1.00 | 21.25 | C |
| ATOM | 2044 | OE1 | GLU | A | 283 | 48.463 | 15.806 | −4.453 | 1.00 | 21.74 | O |
| ATOM | 2045 | OE2 | GLU | A | 283 | 46.399 | 15.240 | −4.852 | 1.00 | 26.34 | O |
| ATOM | 2046 | C | GLU | A | 283 | 47.994 | 20.850 | −3.277 | 1.00 | 16.99 | C |
| ATOM | 2047 | O | GLU | A | 283 | 47.090 | 21.208 | −2.519 | 1.00 | 16.78 | O |
| ATOM | 2048 | N | ILE | A | 284 | 49.260 | 21.208 | −3.109 | 1.00 | 16.34 | N |
| ATOM | 2049 | CA | ILE | A | 284 | 49.645 | 22.147 | −2.057 | 1.00 | 16.23 | C |
| ATOM | 2050 | CB | ILE | A | 284 | 51.182 | 22.296 | −1.977 | 1.00 | 15.68 | C |
| ATOM | 2051 | CG1 | ILE | A | 284 | 51.837 | 20.997 | −1.492 | 1.00 | 15.14 | C |
| ATOM | 2052 | CD1 | ILE | A | 284 | 53.373 | 20.968 | −1.576 | 1.00 | 13.98 | C |
| ATOM | 2053 | CG2 | ILE | A | 284 | 51.552 | 23.488 | −1.074 | 1.00 | 15.67 | C |
| ATOM | 2054 | C | ILE | A | 284 | 49.003 | 23.507 | −2.320 | 1.00 | 16.48 | C |
| ATOM | 2055 | O | ILE | A | 284 | 48.371 | 24.076 | −1.447 | 1.00 | 16.43 | O |
| ATOM | 2056 | N | GLN | A | 285 | 49.162 | 24.009 | −3.539 | 1.00 | 16.57 | N |
| ATOM | 2057 | CA | GLN | A | 285 | 48.677 | 25.335 | −3.872 | 1.00 | 17.32 | C |
| ATOM | 2058 | CB | GLN | A | 285 | 49.376 | 25.867 | −5.124 | 1.00 | 16.61 | C |
| ATOM | 2059 | CG | GLN | A | 285 | 50.848 | 26.173 | −4.858 | 1.00 | 16.82 | C |
| ATOM | 2060 | CD | GLN | A | 285 | 51.485 | 26.941 | −5.984 | 1.00 | 16.61 | C |
| ATOM | 2061 | OE1 | GLN | A | 285 | 51.643 | 26.408 | −7.087 | 1.00 | 16.49 | O |
| ATOM | 2062 | NE2 | GLN | A | 285 | 51.822 | 28.204 | −5.731 | 1.00 | 12.74 | N |
| ATOM | 2063 | C | GLN | A | 285 | 47.153 | 25.426 | −3.998 | 1.00 | 17.59 | C |
| ATOM | 2064 | O | GLN | A | 285 | 46.596 | 26.520 | −3.882 | 1.00 | 17.62 | O |
| ATOM | 2065 | N | ASN | A | 286 | 46.495 | 24.292 | −4.231 | 1.00 | 17.76 | N |
| ATOM | 2066 | CA | ASN | A | 286 | 45.038 | 24.227 | −4.189 | 1.00 | 18.67 | C |
| ATOM | 2067 | CB | ASN | A | 286 | 44.507 | 23.196 | −5.199 | 1.00 | 19.01 | C |
| ATOM | 2068 | CG | ASN | A | 286 | 44.599 | 23.683 | −6.644 | 1.00 | 20.31 | C |
| ATOM | 2069 | OD1 | ASN | A | 286 | 44.581 | 24.890 | −6.923 | 1.00 | 21.12 | O |
| ATOM | 2070 | ND2 | ASN | A | 286 | 44.697 | 22.746 | −7.566 | 1.00 | 21.21 | N |
| ATOM | 2071 | C | ASN | A | 286 | 44.473 | 23.948 | −2.787 | 1.00 | 19.15 | C |
| ATOM | 2072 | O | ASN | A | 286 | 43.253 | 23.959 | −2.585 | 1.00 | 18.66 | O |
| ATOM | 2073 | N | HIS | A | 287 | 45.362 | 23.711 | −1.820 | 1.00 | 19.57 | N |
| ATOM | 2074 | CA | HIS | A | 287 | 44.943 | 23.402 | −0.455 | 1.00 | 19.96 | C |
| ATOM | 2075 | CB | HIS | A | 287 | 46.161 | 23.008 | 0.398 | 1.00 | 20.42 | C |
| ATOM | 2076 | CG | HIS | A | 287 | 45.811 | 22.535 | 1.776 | 1.00 | 20.16 | C |
| ATOM | 2077 | ND1 | HIS | A | 287 | 45.771 | 21.201 | 2.120 | 1.00 | 21.24 | N |
| ATOM | 2078 | CE1 | HIS | A | 287 | 45.417 | 21.085 | 3.388 | 1.00 | 20.50 | C |
| ATOM | 2079 | NE2 | HIS | A | 287 | 45.224 | 22.298 | 3.878 | 1.00 | 21.14 | N |
| ATOM | 2080 | CD2 | HIS | A | 287 | 45.469 | 23.220 | 2.891 | 1.00 | 19.59 | C |
| ATOM | 2081 | C | HIS | A | 287 | 44.212 | 24.613 | 0.140 | 1.00 | 20.17 | C |
| ATOM | 2082 | O | HIS | A | 287 | 44.585 | 25.753 | −0.140 | 1.00 | 20.09 | O |
| ATOM | 2083 | N | PRO | A | 288 | 43.154 | 24.375 | 0.921 | 1.00 | 20.64 | N |
| ATOM | 2084 | CA | PRO | A | 288 | 42.399 | 25.468 | 1.540 | 1.00 | 20.94 | C |
| ATOM | 2085 | CB | PRO | A | 288 | 41.409 | 24.741 | 2.463 | 1.00 | 21.25 | C |
| ATOM | 2086 | CG | PRO | A | 288 | 41.229 | 23.429 | 1.836 | 1.00 | 21.49 | C |
| ATOM | 2087 | CD | PRO | A | 288 | 42.549 | 23.063 | 1.215 | 1.00 | 20.84 | C |
| ATOM | 2088 | C | PRO | A | 288 | 43.263 | 26.449 | 2.323 | 1.00 | 20.62 | C |
| ATOM | 2089 | O | PRO | A | 288 | 42.995 | 27.641 | 2.242 | 1.00 | 21.00 | O |
| ATOM | 2090 | N | TRP | A | 289 | 44.290 | 25.982 | 3.027 | 1.00 | 20.44 | N |
| ATOM | 2091 | CA | TRP | A | 289 | 45.144 | 26.903 | 3.785 | 1.00 | 20.73 | C |
| ATOM | 2092 | CB | TRP | A | 289 | 46.165 | 26.169 | 4.668 | 1.00 | 19.78 | C |
| ATOM | 2093 | CG | TRP | A | 289 | 46.932 | 27.139 | 5.535 | 1.00 | 18.93 | C |
| ATOM | 2094 | CD1 | TRP | A | 289 | 46.469 | 27.795 | 6.646 | 1.00 | 17.79 | C |
| ATOM | 2095 | NE1 | TRP | A | 289 | 47.450 | 28.617 | 7.152 | 1.00 | 17.69 | N |
| ATOM | 2096 | CE2 | TRP | A | 289 | 48.560 | 28.535 | 6.348 | 1.00 | 17.45 | C |
| ATOM | 2097 | CD2 | TRP | A | 289 | 48.265 | 27.614 | 5.316 | 1.00 | 16.35 | C |
| ATOM | 2098 | CE3 | TRP | A | 289 | 49.252 | 27.348 | 4.352 | 1.00 | 16.19 | C |
| ATOM | 2099 | CZ3 | TRP | A | 289 | 50.488 | 27.992 | 4.453 | 1.00 | 14.25 | C |
| ATOM | 2100 | CH2 | TRP | A | 289 | 50.753 | 28.894 | 5.498 | 1.00 | 15.51 | C |
| ATOM | 2101 | CZ2 | TRP | A | 289 | 49.805 | 29.181 | 6.453 | 1.00 | 16.21 | C |
| ATOM | 2102 | C | TRP | A | 289 | 45.868 | 27.924 | 2.897 | 1.00 | 21.43 | C |
| ATOM | 2103 | O | TRP | A | 289 | 46.219 | 29.005 | 3.371 | 1.00 | 21.35 | O |
| ATOM | 2104 | N | MET | A | 290 | 46.081 | 27.575 | 1.627 | 1.00 | 22.16 | N |
| ATOM | 2105 | CA | MET | A | 290 | 46.795 | 28.432 | 0.672 | 1.00 | 23.41 | C |
| ATOM | 2106 | CB | MET | A | 290 | 47.570 | 27.561 | −0.328 | 1.00 | 23.44 | C |
| ATOM | 2107 | CG | MET | A | 290 | 48.631 | 26.687 | 0.341 | 1.00 | 23.18 | C |
| ATOM | 2108 | SD | MET | A | 290 | 50.289 | 27.282 | −0.016 | 1.00 | 24.31 | S |
| ATOM | 2109 | CE | MET | A | 290 | 50.282 | 28.805 | 0.810 | 1.00 | 21.67 | C |
| ATOM | 2110 | C | MET | A | 290 | 45.935 | 29.456 | −0.093 | 1.00 | 24.66 | C |
| ATOM | 2111 | O | MET | A | 290 | 46.465 | 30.229 | −0.901 | 1.00 | 24.82 | O |
| ATOM | 2112 | N | GLN | A | 291 | 44.627 | 29.472 | 0.161 | 1.00 | 25.63 | N |
| ATOM | 2113 | CA | GLN | A | 291 | 43.725 | 30.390 | −0.541 | 1.00 | 27.47 | C |
| ATOM | 2114 | CB | GLN | A | 291 | 42.263 | 29.932 | −0.399 | 1.00 | 28.12 | C |
| ATOM | 2115 | CG | GLN | A | 291 | 41.931 | 28.612 | −1.133 | 1.00 | 30.95 | C |
| ATOM | 2116 | CD | GLN | A | 291 | 42.797 | 28.376 | −2.378 | 1.00 | 35.68 | C |
| ATOM | 2117 | OE1 | GLN | A | 291 | 42.599 | 29.038 | −3.414 | 1.00 | 37.06 | O |
| ATOM | 2118 | NE2 | GLN | A | 291 | 43.766 | 27.441 | −2.277 | 1.00 | 34.40 | N |
| ATOM | 2119 | C | GLN | A | 291 | 43.879 | 31.844 | −0.094 | 1.00 | 27.73 | C |
| ATOM | 2120 | O | GLN | A | 291 | 44.222 | 32.122 | 1.059 | 1.00 | 28.09 | O |
| ATOM | 2121 | N | ASP | A | 292 | 43.651 | 32.765 | −1.026 | 1.00 | 28.22 | N |
| ATOM | 2122 | CA | ASP | A | 292 | 43.678 | 34.207 | −0.750 | 1.00 | 28.34 | C |
| ATOM | 2123 | CB | ASP | A | 292 | 42.553 | 34.596 | 0.224 | 1.00 | 29.02 | C |
| ATOM | 2124 | CG | ASP | A | 292 | 41.176 | 34.236 | −0.308 | 1.00 | 31.45 | C |
| ATOM | 2125 | OD1 | ASP | A | 292 | 40.817 | 34.731 | −1.402 | 1.00 | 33.30 | O |
| ATOM | 2126 | OD2 | ASP | A | 292 | 40.400 | 33.452 | 0.291 | 1.00 | 34.49 | O |
| ATOM | 2127 | C | ASP | A | 292 | 45.027 | 34.718 | −0.245 | 1.00 | 27.65 | C |
| ATOM | 2128 | O | ASP | A | 292 | 45.098 | 35.446 | 0.761 | 1.00 | 27.28 | O |
| ATOM | 2129 | N | VAL | A | 293 | 46.094 | 34.334 | −0.946 | 1.00 | 26.88 | N |
| ATOM | 2130 | CA | VAL | A | 293 | 47.429 | 34.808 | −0.622 | 1.00 | 25.72 | C |
| ATOM | 2131 | CB | VAL | A | 293 | 48.538 | 34.025 | −1.396 | 1.00 | 26.36 | C |
| ATOM | 2132 | CG1 | VAL | A | 293 | 48.546 | 34.374 | −2.881 | 1.00 | 26.33 | C |
| ATOM | 2133 | CG2 | VAL | A | 293 | 49.912 | 34.299 | −0.799 | 1.00 | 24.82 | C |
| ATOM | 2134 | C | VAL | A | 293 | 47.550 | 36.311 | −0.877 | 1.00 | 25.50 | C |
| ATOM | 2135 | O | VAL | A | 293 | 47.007 | 36.824 | −1.848 | 1.00 | 24.70 | O |
| ATOM | 2136 | N | LEU | A | 294 | 48.261 | 37.004 | 0.009 | 1.00 | 25.07 | N |
| ATOM | 2137 | CA | LEU | A | 294 | 48.630 | 38.392 | −0.226 | 1.00 | 25.15 | C |
| ATOM | 2138 | CB | LEU | A | 294 | 49.280 | 38.998 | 1.017 | 1.00 | 24.83 | C |
| ATOM | 2139 | CG | LEU | A | 294 | 48.500 | 39.140 | 2.329 | 1.00 | 24.86 | C |
| ATOM | 2140 | CD1 | LEU | A | 294 | 49.412 | 39.783 | 3.371 | 1.00 | 22.85 | C |
| ATOM | 2141 | CD2 | LEU | A | 294 | 47.199 | 39.941 | 2.148 | 1.00 | 24.42 | C |
| ATOM | 2142 | C | LEU | A | 294 | 49.621 | 38.487 | −1.384 | 1.00 | 25.66 | C |
| ATOM | 2143 | O | LEU | A | 294 | 50.437 | 37.585 | −1.598 | 1.00 | 25.15 | O |
| ATOM | 2144 | N | LEU | A | 295 | 49.539 | 39.585 | −2.128 | 1.00 | 26.06 | N |
| ATOM | 2145 | CA | LEU | A | 295 | 50.587 | 39.950 | −3.071 | 1.00 | 26.73 | C |
| ATOM | 2146 | CB | LEU | A | 295 | 50.122 | 41.106 | −3.981 | 1.00 | 27.23 | C |
| ATOM | 2147 | CG | LEU | A | 295 | 48.775 | 40.944 | −4.717 | 1.00 | 29.21 | C |
| ATOM | 2148 | CD1 | LEU | A | 295 | 48.330 | 42.237 | −5.412 | 1.00 | 31.53 | C |
| ATOM | 2149 | CD2 | LEU | A | 295 | 48.829 | 39.801 | −5.721 | 1.00 | 31.44 | C |
| ATOM | 2150 | C | LEU | A | 295 | 51.841 | 40.337 | −2.265 | 1.00 | 26.44 | C |
| ATOM | 2151 | O | LEU | A | 295 | 51.729 | 40.733 | −1.103 | 1.00 | 25.11 | O |
| ATOM | 2152 | N | PRO | A | 296 | 53.028 | 40.170 | −2.851 | 1.00 | 27.04 | N |
| ATOM | 2153 | CA | PRO | A | 296 | 54.277 | 40.535 | −2.164 | 1.00 | 27.59 | C |
| ATOM | 2154 | CB | PRO | A | 296 | 55.331 | 40.358 | −3.250 | 1.00 | 27.65 | C |
| ATOM | 2155 | CG | PRO | A | 296 | 54.772 | 39.247 | −4.091 | 1.00 | 27.68 | C |
| ATOM | 2156 | CD | PRO | A | 296 | 53.292 | 39.564 | −4.171 | 1.00 | 26.80 | C |
| ATOM | 2157 | C | PRO | A | 296 | 54.265 | 41.964 | −1.623 | 1.00 | 28.49 | C |
| ATOM | 2158 | O | PRO | A | 296 | 54.608 | 42.151 | −0.459 | 1.00 | 28.36 | O |
| ATOM | 2159 | N | GLN | A | 297 | 53.854 | 42.942 | −2.430 | 1.00 | 29.34 | N |
| ATOM | 2160 | CA | GLN | A | 297 | 53.801 | 44.328 | −1.960 | 1.00 | 30.65 | C |
| ATOM | 2161 | CB | GLN | A | 297 | 53.467 | 45.305 | −3.102 | 1.00 | 31.10 | C |
| ATOM | 2162 | CG | GLN | A | 297 | 53.783 | 46.766 | −2.787 | 1.00 | 33.83 | C |
| ATOM | 2163 | CD | GLN | A | 297 | 55.239 | 46.993 | −2.374 | 1.00 | 37.47 | C |
| ATOM | 2164 | OE1 | GLN | A | 297 | 56.158 | 46.742 | −3.153 | 1.00 | 38.85 | O |
| ATOM | 2165 | NE2 | GLN | A | 297 | 55.444 | 47.468 | −1.147 | 1.00 | 39.03 | N |
| ATOM | 2166 | C | GLN | A | 297 | 52.830 | 44.490 | −0.782 | 1.00 | 30.41 | C |
| ATOM | 2167 | O | GLN | A | 297 | 53.174 | 45.125 | 0.210 | 1.00 | 30.58 | O |
| ATOM | 2168 | N | GLU | A | 298 | 51.640 | 43.900 | −0.891 | 1.00 | 30.38 | N |
| ATOM | 2169 | CA | GLU | A | 298 | 50.699 | 43.839 | 0.235 | 1.00 | 30.73 | C |
| ATOM | 2170 | CB | GLU | A | 298 | 49.479 | 42.981 | −0.103 | 1.00 | 31.10 | C |
| ATOM | 2171 | CG | GLU | A | 298 | 48.534 | 43.558 | −1.141 | 1.00 | 33.59 | C |
| ATOM | 2172 | CD | GLU | A | 298 | 47.270 | 42.722 | −1.289 | 1.00 | 37.83 | C |
| ATOM | 2173 | OE1 | GLU | A | 298 | 47.369 | 41.480 | −1.435 | 1.00 | 36.99 | O |
| ATOM | 2174 | OE2 | GLU | A | 298 | 46.166 | 43.313 | −1.272 | 1.00 | 40.82 | O |
| ATOM | 2175 | C | GLU | A | 298 | 51.378 | 43.269 | 1.485 | 1.00 | 30.06 | C |
| ATOM | 2176 | O | GLU | A | 298 | 51.258 | 43.837 | 2.577 | 1.00 | 30.09 | O |
| ATOM | 2177 | N | THR | A | 299 | 52.096 | 42.156 | 1.301 | 1.00 | 28.83 | N |
| ATOM | 2178 | CA | THR | A | 299 | 52.857 | 41.496 | 2.360 | 1.00 | 27.61 | C |
| ATOM | 2179 | CB | THR | A | 299 | 53.619 | 40.265 | 1.782 | 1.00 | 27.45 | C |
| ATOM | 2180 | OG1 | THR | A | 299 | 52.690 | 39.346 | 1.192 | 1.00 | 25.03 | O |
| ATOM | 2181 | CG2 | THR | A | 299 | 54.269 | 39.447 | 2.897 | 1.00 | 26.87 | C |
| ATOM | 2182 | C | THR | A | 299 | 53.840 | 42.442 | 3.062 | 1.00 | 27.70 | C |
| ATOM | 2183 | O | THR | A | 299 | 53.890 | 42.487 | 4.289 | 1.00 | 27.42 | O |
| ATOM | 2184 | N | ALA | A | 300 | 54.626 | 43.177 | 2.278 | 1.00 | 27.78 | N |
| ATOM | 2185 | CA | ALA | A | 300 | 55.622 | 44.093 | 2.819 | 1.00 | 28.26 | C |
| ATOM | 2186 | CB | ALA | A | 300 | 56.517 | 44.627 | 1.706 | 1.00 | 28.09 | C |
| ATOM | 2187 | C | ALA | A | 300 | 54.972 | 45.250 | 3.588 | 1.00 | 28.73 | C |
| ATOM | 2188 | O | ALA | A | 300 | 55.444 | 45.636 | 4.658 | 1.00 | 28.08 | O |
| ATOM | 2189 | N | GLU | A | 301 | 53.884 | 45.785 | 3.040 | 1.00 | 29.58 | N |
| ATOM | 2190 | CA | GLU | A | 301 | 53.169 | 46.894 | 3.668 | 1.00 | 31.18 | C |
| ATOM | 2191 | CB | GLU | A | 301 | 52.063 | 47.433 | 2.738 | 1.00 | 31.45 | C |
| ATOM | 2192 | CG | GLU | A | 301 | 52.592 | 48.250 | 1.555 | 1.00 | 34.28 | C |
| ATOM | 2193 | CD | GLU | A | 301 | 51.553 | 48.517 | 0.460 | 1.00 | 37.15 | C |
| ATOM | 2194 | OE1 | GLU | A | 301 | 50.659 | 47.668 | 0.219 | 1.00 | 37.47 | O |
| ATOM | 2195 | OE2 | GLU | A | 301 | 51.642 | 49.589 | −0.177 | 1.00 | 38.55 | O |
| ATOM | 2196 | C | GLU | A | 301 | 52.610 | 46.488 | 5.042 | 1.00 | 31.10 | C |
| ATOM | 2197 | O | GLU | A | 301 | 52.779 | 47.211 | 6.019 | 1.00 | 31.18 | O |
| ATOM | 2198 | N | ILE | A | 302 | 51.989 | 45.311 | 5.107 | 1.00 | 31.28 | N |
| ATOM | 2199 | CA | ILE | A | 302 | 51.371 | 44.823 | 6.340 | 1.00 | 31.59 | C |
| ATOM | 2200 | CB | ILE | A | 302 | 50.284 | 43.767 | 6.019 | 1.00 | 31.30 | C |
| ATOM | 2201 | CG1 | ILE | A | 302 | 49.201 | 44.378 | 5.115 | 1.00 | 31.02 | C |
| ATOM | 2202 | CD1 | ILE | A | 302 | 48.293 | 43.357 | 4.435 | 1.00 | 29.87 | C |
| ATOM | 2203 | CG2 | ILE | A | 302 | 49.673 | 43.195 | 7.311 | 1.00 | 30.80 | C |
| ATOM | 2204 | C | ILE | A | 302 | 52.384 | 44.286 | 7.373 | 1.00 | 32.02 | C |
| ATOM | 2205 | O | ILE | A | 302 | 52.263 | 44.577 | 8.560 | 1.00 | 31.80 | O |
| ATOM | 2206 | N | HIS | A | 303 | 53.391 | 43.544 | 6.909 | 1.00 | 32.60 | N |
| ATOM | 2207 | CA | HIS | A | 303 | 54.253 | 42.750 | 7.790 | 1.00 | 33.26 | C |
| ATOM | 2208 | CB | HIS | A | 303 | 54.176 | 41.282 | 7.379 | 1.00 | 32.20 | C |
| ATOM | 2209 | CG | HIS | A | 303 | 52.832 | 40.662 | 7.606 | 1.00 | 29.68 | C |
| ATOM | 2210 | ND1 | HIS | A | 303 | 52.385 | 40.293 | 8.857 | 1.00 | 27.26 | N |
| ATOM | 2211 | CE1 | HIS | A | 303 | 51.171 | 39.780 | 8.755 | 1.00 | 27.29 | C |
| ATOM | 2212 | NE2 | HIS | A | 303 | 50.819 | 39.796 | 7.481 | 1.00 | 26.60 | N |
| ATOM | 2213 | CD2 | HIS | A | 303 | 51.839 | 40.346 | 6.743 | 1.00 | 26.43 | C |
| ATOM | 2214 | C | HIS | A | 303 | 55.723 | 43.174 | 7.849 | 1.00 | 35.09 | C |
| ATOM | 2215 | O | HIS | A | 303 | 56.435 | 42.838 | 8.796 | 1.00 | 34.73 | O |
| ATOM | 2216 | N | LEU | A | 304 | 56.181 | 43.889 | 6.827 | 1.00 | 37.42 | N |
| ATOM | 2217 | CA | LEU | A | 304 | 57.588 | 44.256 | 6.724 | 1.00 | 40.17 | C |
| ATOM | 2218 | CB | LEU | A | 304 | 58.187 | 43.707 | 5.421 | 1.00 | 39.43 | C |
| ATOM | 2219 | CG | LEU | A | 304 | 58.574 | 42.224 | 5.234 | 1.00 | 38.74 | C |
| ATOM | 2220 | CD1 | LEU | A | 304 | 57.830 | 41.238 | 6.125 | 1.00 | 34.54 | C |
| ATOM | 2221 | CD2 | LEU | A | 304 | 58.465 | 41.807 | 3.760 | 1.00 | 35.74 | C |
| ATOM | 2222 | C | LEU | A | 304 | 57.751 | 45.774 | 6.796 | 1.00 | 42.83 | C |
| ATOM | 2223 | O | LEU | A | 304 | 58.861 | 46.286 | 6.661 | 1.00 | 43.07 | O |
| ATOM | 2224 | N | HIS | A | 305 | 56.629 | 46.462 | 7.033 | 1.00 | 46.30 | N |
| ATOM | 2225 | CA | HIS | A | 305 | 56.516 | 47.933 | 7.107 | 1.00 | 49.71 | C |
| ATOM | 2226 | CB | HIS | A | 305 | 56.747 | 48.459 | 8.545 | 1.00 | 50.37 | C |
| ATOM | 2227 | CG | HIS | A | 305 | 58.085 | 48.106 | 9.125 | 1.00 | 53.30 | C |
| ATOM | 2228 | ND1 | HIS | A | 305 | 58.344 | 46.886 | 9.716 | 1.00 | 56.26 | N |
| ATOM | 2229 | CE1 | HIS | A | 305 | 59.597 | 46.860 | 10.138 | 1.00 | 57.48 | C |
| ATOM | 2230 | NE2 | HIS | A | 305 | 60.159 | 48.022 | 9.847 | 1.00 | 57.58 | N |
| ATOM | 2231 | CD2 | HIS | A | 305 | 59.234 | 48.820 | 9.216 | 1.00 | 56.22 | C |
| ATOM | 2232 | C | HIS | A | 305 | 57.339 | 48.707 | 6.063 | 1.00 | 50.97 | C |
| ATOM | 2233 | O | HIS | A | 305 | 58.423 | 49.221 | 6.359 | 1.00 | 51.59 | O |
| ATOM | 2234 | N | SER | A | 306 | 56.795 | 48.799 | 4.850 | 1.00 | 52.46 | N |
| ATOM | 2235 | CA | SER | A | 306 | 57.518 | 49.345 | 3.693 | 1.00 | 53.65 | C |
| ATOM | 2236 | CB | SER | A | 306 | 56.768 | 49.009 | 2.401 | 1.00 | 53.75 | C |
| ATOM | 2237 | OG | SER | A | 306 | 57.340 | 47.873 | 1.784 | 1.00 | 54.33 | O |
| ATOM | 2238 | C | SER | A | 306 | 57.820 | 50.851 | 3.763 | 1.00 | 54.12 | C |
| ATOM | 2239 | O | SER | A | 306 | 58.964 | 51.291 | 3.600 | 1.00 | 54.51 | O |
| ATOM | 2240 | OXT | SER | A | 306 | 56.943 | 51.696 | 3.974 | 1.00 | 54.48 | O |
| ATOM | 2241 | O1A | ANP | L | 1 | 74.739 | 30.562 | −0.833 | 1.00 | 18.02 | O |
| ATOM | 2242 | PA | ANP | L | 1 | 74.774 | 30.444 | 0.630 | 1.00 | 19.01 | P |
| ATOM | 2243 | O2A | ANP | L | 1 | 73.576 | 29.828 | 1.256 | 1.00 | 17.67 | O |
| ATOM | 2244 | O3A | ANP | L | 1 | 76.090 | 29.652 | 0.938 | 1.00 | 19.50 | O |
| ATOM | 2245 | PB | ANP | L | 1 | 76.391 | 28.426 | 1.887 | 1.00 | 21.38 | P |
| ATOM | 2246 | O1B | ANP | L | 1 | 77.321 | 27.642 | 1.069 | 1.00 | 18.82 | O |
| ATOM | 2247 | O2B | ANP | L | 1 | 77.348 | 29.007 | 2.991 | 1.00 | 24.75 | O |
| ATOM | 2248 | N3B | ANP | L | 1 | 75.190 | 27.617 | 2.664 | 1.00 | 20.02 | N |
| ATOM | 2249 | PG | ANP | L | 1 | 73.526 | 27.764 | 2.959 | 1.00 | 31.62 | P |
| ATOM | 2250 | O3G | ANP | L | 1 | 73.022 | 29.016 | 3.938 | 1.00 | 19.18 | O |
| ATOM | 2251 | O2G | ANP | L | 1 | 73.054 | 27.935 | 1.600 | 1.00 | 20.72 | O |
| ATOM | 2252 | O1G | ANP | L | 1 | 72.907 | 26.389 | 3.404 | 1.00 | 20.30 | O |
| ATOM | 2253 | O5* | ANP | L | 1 | 74.945 | 31.880 | 1.324 | 1.00 | 18.06 | O |
| ATOM | 2254 | C5* | ANP | L | 1 | 75.248 | 31.923 | 2.714 | 1.00 | 17.09 | C |
| ATOM | 2255 | C4* | ANP | L | 1 | 74.482 | 33.091 | 3.324 | 1.00 | 18.01 | C |
| ATOM | 2256 | O4* | ANP | L | 1 | 74.771 | 34.292 | 2.621 | 1.00 | 19.09 | O |
| ATOM | 2257 | C1* | ANP | L | 1 | 73.660 | 35.124 | 2.442 | 1.00 | 17.31 | C |
| ATOM | 2258 | C2* | ANP | L | 1 | 72.535 | 34.397 | 3.160 | 1.00 | 18.01 | C |
| ATOM | 2259 | O2* | ANP | L | 1 | 72.451 | 34.900 | 4.487 | 1.00 | 19.01 | O |
| ATOM | 2260 | C3* | ANP | L | 1 | 72.983 | 32.937 | 3.178 | 1.00 | 17.74 | C |
| ATOM | 2261 | O3* | ANP | L | 1 | 72.429 | 32.083 | 4.163 | 1.00 | 17.16 | O |
| ATOM | 2262 | N9 | ANP | L | 1 | 73.486 | 35.319 | 0.979 | 1.00 | 17.49 | N |
| ATOM | 2263 | C8 | ANP | L | 1 | 73.739 | 34.403 | −0.019 | 1.00 | 15.85 | C |
| ATOM | 2264 | N7 | ANP | L | 1 | 73.458 | 34.943 | −1.228 | 1.00 | 14.30 | N |
| ATOM | 2265 | C5 | ANP | L | 1 | 73.025 | 36.193 | −1.045 | 1.00 | 15.53 | C |
| ATOM | 2266 | C6 | ANP | L | 1 | 72.607 | 37.177 | −1.929 | 1.00 | 16.13 | C |
| ATOM | 2267 | N6 | ANP | L | 1 | 72.542 | 36.951 | −3.254 | 1.00 | 14.38 | N |
| ATOM | 2268 | C4 | ANP | L | 1 | 73.039 | 36.448 | 0.334 | 1.00 | 15.81 | C |
| ATOM | 2269 | N3 | ANP | L | 1 | 72.632 | 37.646 | 0.795 | 1.00 | 17.04 | N |
| ATOM | 2270 | C2 | ANP | L | 1 | 72.219 | 38.650 | −0.068 | 1.00 | 17.00 | C |
| ATOM | 2271 | N1 | ANP | L | 1 | 72.213 | 38.406 | −1.417 | 1.00 | 16.49 | N |
| ATOM | 2272 | O | HOH | W | 1 | 63.572 | 15.756 | 8.058 | 1.00 | 26.33 | O |
| ATOM | 2273 | O | HOH | W | 2 | 61.017 | 10.214 | −2.362 | 1.00 | 24.27 | O |
| ATOM | 2274 | O | HOH | W | 3 | 54.457 | 23.038 | −11.444 | 1.00 | 30.92 | O |
| ATOM | 2275 | O | HOH | W | 4 | 63.756 | 21.549 | −5.585 | 1.00 | 27.71 | O |
| ATOM | 2276 | O | HOH | W | 5 | 63.196 | 11.516 | −9.068 | 1.00 | 26.46 | O |
| ATOM | 2277 | O | HOH | W | 6 | 58.424 | 12.040 | −6.552 | 1.00 | 34.61 | O |
| ATOM | 2278 | O | HOH | W | 7 | 54.593 | 37.425 | 12.022 | 1.00 | 32.21 | O |
| ATOM | 2279 | O | HOH | W | 8 | 71.368 | 23.298 | −3.142 | 1.00 | 29.05 | O |
| ATOM | 2280 | O | HOH | W | 9 | 64.911 | 20.478 | −0.663 | 1.00 | 26.42 | O |
| ATOM | 2281 | O | HOH | W | 10 | 43.132 | 26.500 | 18.254 | 1.00 | 34.21 | O |
| ATOM | 2282 | O | HOH | W | 11 | 64.667 | 17.153 | −3.465 | 1.00 | 26.68 | O |
| ATOM | 2283 | O | HOH | W | 12 | 75.478 | 24.941 | 1.494 | 1.00 | 29.16 | O |
| ATOM | 2284 | O | HOH | W | 13 | 63.267 | 18.804 | 11.098 | 1.00 | 24.60 | O |
| ATOM | 2285 | O | HOH | W | 14 | 47.333 | 35.497 | 10.172 | 1.00 | 39.07 | O |
| ATOM | 2286 | O | HOH | W | 15 | 41.592 | 25.798 | 13.188 | 1.00 | 29.60 | O |
| ATOM | 2287 | O | HOH | W | 16 | 46.216 | 35.678 | 3.186 | 1.00 | 30.38 | O |
| ATOM | 2288 | O | HOH | W | 17 | 73.656 | 23.760 | −0.205 | 1.00 | 33.83 | O |
| ATOM | 2289 | O | HOH | W | 18 | 54.975 | 14.884 | −3.637 | 1.00 | 27.43 | O |
| ATOM | 2290 | O | HOH | W | 19 | 58.350 | 33.360 | −6.094 | 1.00 | 29.01 | O |
| ATOM | 2291 | O | HOH | W | 20 | 58.458 | 11.832 | 15.075 | 1.00 | 34.06 | O |
| ATOM | 2292 | O | HOH | W | 21 | 48.299 | 24.286 | 17.311 | 1.00 | 29.81 | O |
| ATOM | 2293 | O | HOH | W | 22 | 67.356 | 21.562 | 3.234 | 1.00 | 31.16 | O |
| ATOM | 2294 | O | HOH | W | 23 | 84.186 | 28.990 | 2.689 | 1.00 | 31.81 | O |
| ATOM | 2295 | O | HOH | W | 24 | 43.050 | 31.228 | 3.507 | 1.00 | 47.48 | O |
| ATOM | 2296 | O | HOH | W | 25 | 88.316 | 32.615 | −4.360 | 1.00 | 32.51 | O |
| ATOM | 2297 | O | HOH | W | 26 | 71.447 | 43.185 | −7.672 | 1.00 | 43.19 | O |
| ATOM | 2298 | O | HOH | W | 27 | 64.646 | 19.993 | −3.620 | 1.00 | 28.98 | O |
| ATOM | 2299 | O | HOH | W | 28 | 71.618 | 43.781 | 0.688 | 1.00 | 40.30 | O |
| ATOM | 2300 | O | HOH | W | 29 | 70.325 | 37.710 | 6.677 | 1.00 | 31.97 | O |
| ATOM | 2301 | O | HOH | W | 30 | 71.184 | 18.590 | 9.525 | 1.00 | 35.40 | O |
| ATOM | 2302 | O | HOH | W | 31 | 53.890 | 12.402 | −3.734 | 1.00 | 32.44 | O |
| ATOM | 2303 | O | HOH | W | 32 | 52.246 | 19.524 | −9.419 | 1.00 | 35.84 | O |
| ATOM | 2304 | O | HOH | W | 33 | 40.639 | 26.398 | 16.837 | 1.00 | 35.65 | O |
| ATOM | 2305 | O | HOH | W | 34 | 60.620 | 13.344 | −8.811 | 1.00 | 44.92 | O |
| ATOM | 2306 | O | HOH | W | 35 | 75.110 | 44.117 | 2.424 | 1.00 | 40.04 | O |
| ATOM | 2307 | O | HOH | W | 36 | 74.471 | 37.990 | 7.461 | 1.00 | 40.83 | O |
| ATOM | 2308 | O | HOH | W | 37 | 59.228 | 35.799 | −5.068 | 1.00 | 33.92 | O |
| ATOM | 2309 | O | HOH | W | 38 | 57.123 | 17.309 | 16.065 | 1.00 | 40.82 | O |
| ATOM | 2310 | O | HOH | W | 39 | 73.994 | 32.523 | −2.675 | 1.00 | 33.30 | O |
| ATOM | 2311 | O | HOH | W | 40 | 69.993 | 44.693 | 3.957 | 1.00 | 50.42 | O |
| ATOM | 2312 | O | HOH | W | 41 | 65.864 | 18.120 | 0.377 | 1.00 | 37.77 | O |
| ATOM | 2313 | O | HOH | W | 42 | 48.834 | 35.741 | 2.440 | 1.00 | 31.77 | O |
| ATOM | 2314 | O | HOH | W | 43 | 52.185 | 7.956 | 4.576 | 1.00 | 38.71 | O |
| ATOM | 2315 | O | HOH | W | 44 | 64.765 | 11.133 | −15.777 | 1.00 | 33.65 | O |
| ATOM | 2316 | O | HOH | W | 45 | 48.197 | 17.129 | −9.023 | 1.00 | 35.25 | O |
| ATOM | 2317 | O | HOH | W | 46 | 71.559 | 9.704 | −7.782 | 1.00 | 42.63 | O |
| ATOM | 2318 | O | HOH | W | 47 | 72.838 | 31.092 | −4.833 | 1.00 | 32.07 | O |
| ATOM | 2319 | O | HOH | W | 48 | 54.340 | 33.741 | −9.311 | 1.00 | 38.01 | O |
| ATOM | 2320 | O | HOH | W | 49 | 54.223 | 11.905 | 9.111 | 1.00 | 33.22 | O |
| ATOM | 2321 | O | HOH | W | 50 | 52.887 | 36.641 | −1.776 | 1.00 | 38.05 | O |
| ATOM | 2322 | O | HOH | W | 51 | 58.033 | 32.102 | 18.276 | 1.00 | 41.20 | O |
| ATOM | 2323 | O | HOH | W | 52 | 58.764 | 11.092 | 17.987 | 1.00 | 35.48 | O |
| ATOM | 2324 | O | HOH | W | 53 | 56.210 | 29.247 | −10.737 | 1.00 | 40.52 | O |
| ATOM | 2325 | O | HOH | W | 54 | 75.583 | 29.566 | 5.684 | 1.00 | 42.12 | O |
| ATOM | 2326 | O | HOH | W | 55 | 82.299 | 27.704 | 4.152 | 1.00 | 43.41 | O |
| ATOM | 2327 | O | HOH | W | 56 | 61.670 | 6.087 | 15.210 | 1.00 | 42.46 | O |
| ATOM | 2328 | O | HOH | W | 57 | 41.909 | 26.005 | 9.492 | 1.00 | 38.52 | O |
| ATOM | 2329 | O | HOH | W | 58 | 72.941 | 15.417 | 4.788 | 1.00 | 53.05 | O |
| ATOM | 2330 | O | HOH | W | 59 | 56.478 | 27.573 | −12.787 | 1.00 | 37.09 | O |
| ATOM | 2331 | O | HOH | W | 60 | 83.158 | 40.743 | 6.932 | 1.00 | 41.95 | O |
| ATOM | 2332 | O | HOH | W | 61 | 44.574 | 20.141 | −2.416 | 1.00 | 38.16 | O |
| ATOM | 2333 | O | HOH | W | 62 | 51.818 | 13.854 | 13.409 | 1.00 | 36.87 | O |
| ATOM | 2334 | O | HOH | W | 63 | 56.901 | 22.491 | −11.879 | 1.00 | 46.71 | O |
| ATOM | 2335 | O | HOH | W | 64 | 46.066 | 31.890 | −3.335 | 1.00 | 46.54 | O |
| ATOM | 2336 | O | HOH | W | 65 | 46.390 | 17.471 | 11.120 | 1.00 | 41.50 | O |
| ATOM | 2337 | O | HOH | W | 66 | 73.021 | 18.047 | 7.679 | 1.00 | 45.56 | O |
| ATOM | 2338 | O | HOH | W | 67 | 56.272 | 6.117 | −3.207 | 1.00 | 47.16 | O |
| ATOM | 2339 | O | HOH | W | 68 | 78.807 | 46.222 | 0.194 | 1.00 | 43.86 | O |
| ATOM | 2340 | O | HOH | W | 69 | 70.343 | 14.512 | −11.989 | 1.00 | 41.06 | O |
| ATOM | 2341 | O | HOH | W | 70 | 43.908 | 38.440 | 0.670 | 1.00 | 53.02 | O |
| ATOM | 2342 | O | HOH | W | 71 | 40.352 | 28.430 | 2.051 | 1.00 | 45.97 | O |
| ATOM | 2343 | O | HOH | W | 72 | 44.496 | 19.095 | 11.235 | 1.00 | 54.47 | O |
| ATOM | 2344 | O | HOH | W | 73 | 47.165 | 15.788 | 6.720 | 1.00 | 41.01 | O |
| ATOM | 2345 | O | HOH | W | 74 | 56.445 | 43.287 | −5.157 | 1.00 | 44.15 | O |
| ATOM | 2346 | O | HOH | W | 75 | 73.363 | 25.539 | −17.736 | 1.00 | 50.03 | O |
| ATOM | 2347 | O | HOH | W | 76 | 67.665 | 14.838 | −15.990 | 1.00 | 47.37 | O |
| ATOM | 2348 | O | HOH | W | 77 | 77.512 | 31.796 | 8.477 | 1.00 | 43.71 | O |
| ATOM | 2349 | O | HOH | W | 78 | 64.562 | 45.570 | −0.458 | 1.00 | 42.82 | O |
| ATOM | 2350 | O | HOH | W | 79 | 72.601 | 12.906 | 5.087 | 1.00 | 41.65 | O |
| ATOM | 2351 | O | HOH | W | 80 | 64.569 | 29.017 | 13.834 | 1.00 | 46.57 | O |
| ATOM | 2352 | O | HOH | W | 81 | 58.851 | 5.715 | −3.038 | 1.00 | 36.10 | O |
| ATOM | 2353 | O | HOH | W | 82 | 66.378 | 16.370 | −1.785 | 1.00 | 36.18 | O |
| ATOM | 2354 | O | HOH | W | 83 | 52.161 | 13.315 | 8.870 | 1.00 | 38.72 | O |
| ATOM | 2355 | O | HOH | W | 84 | 84.302 | 27.201 | −0.028 | 1.00 | 44.09 | O |
| ATOM | 2356 | O | HOH | W | 85 | 49.501 | 13.263 | −4.243 | 1.00 | 40.50 | O |
| ATOM | 2357 | O | HOH | W | 86 | 63.118 | 41.154 | −5.640 | 1.00 | 44.61 | O |
| ATOM | 2358 | O | HOH | W | 87 | 75.334 | 10.847 | −0.667 | 1.00 | 41.03 | O |
| ATOM | 2359 | O | HOH | W | 88 | 51.946 | 9.440 | 7.089 | 1.00 | 44.13 | O |
| ATOM | 2360 | O | HOH | W | 89 | 46.051 | 15.476 | 9.731 | 1.00 | 44.74 | O |
| ATOM | 2361 | O | HOH | W | 90 | 60.662 | 7.651 | −3.345 | 1.00 | 33.21 | O |
| ATOM | 2362 | O | HOH | W | 91 | 78.926 | 37.602 | 8.589 | 1.00 | 45.81 | O |
| ATOM | 2363 | O | HOH | W | 92 | 83.687 | 38.788 | 8.645 | 1.00 | 42.46 | O |
| ATOM | 2364 | O | HOH | W | 93 | 65.774 | 37.305 | −9.200 | 1.00 | 42.61 | O |
| ATOM | 2365 | O | HOH | W | 94 | 48.890 | 32.798 | 13.190 | 1.00 | 44.81 | O |
| ATOM | 2366 | O | HOH | W | 95 | 71.057 | 6.982 | 7.124 | 1.00 | 46.01 | O |
| ATOM | 2367 | O | HOH | W | 96 | 73.156 | 42.259 | 2.367 | 1.00 | 41.64 | O |
| ATOM | 2368 | O | HOH | W | 97 | 56.031 | 35.393 | 16.920 | 1.00 | 47.09 | O |
| ATOM | 2369 | O | HOH | W | 98 | 90.130 | 23.863 | −3.527 | 1.00 | 56.29 | O |
| ATOM | 2370 | O | HOH | W | 99 | 64.199 | 16.375 | 1.499 | 1.00 | 32.31 | O |
| ATOM | 2371 | O | HOH | W | 100 | 52.185 | 30.882 | 13.804 | 1.00 | 45.03 | O |
| ATOM | 2372 | O | HOH | W | 101 | 78.245 | 25.957 | −3.060 | 1.00 | 37.82 | O |
| ATOM | 2373 | O | HOH | W | 102 | 70.395 | 32.498 | −12.472 | 1.00 | 40.91 | O |
| ATOM | 2374 | O | HOH | W | 103 | 76.497 | 26.635 | −1.230 | 1.00 | 45.90 | O |
| ATOM | 2375 | O | HOH | W | 104 | 53.869 | 39.933 | 10.992 | 1.00 | 48.40 | O |
| ATOM | 2376 | O | HOH | W | 105 | 52.957 | 42.953 | −5.317 | 1.00 | 43.64 | O |
| ATOM | 2377 | O | HOH | W | 106 | 81.062 | 46.768 | −1.405 | 1.00 | 51.11 | O |
| ATOM | 2378 | O | HOH | W | 107 | 85.023 | 38.607 | −2.261 | 1.00 | 44.62 | O |
| ATOM | 2379 | O | HOH | W | 108 | 55.351 | 15.949 | −6.982 | 1.00 | 56.30 | O |
| ATOM | 2380 | O | HOH | W | 109 | 72.893 | 16.276 | −11.510 | 1.00 | 40.24 | O |
| ATOM | 2381 | O | HOH | W | 110 | 64.150 | 29.039 | 11.361 | 1.00 | 44.87 | O |
| ATOM | 2382 | O | HOH | W | 111 | 70.497 | 11.613 | 14.584 | 1.00 | 42.70 | O |
| ATOM | 2383 | O | HOH | W | 112 | 47.743 | 30.590 | 14.200 | 1.00 | 41.84 | O |
| ATOM | 2384 | O | HOH | W | 113 | 67.986 | 19.239 | 2.487 | 1.00 | 45.28 | O |
| ATOM | 2385 | O | HOH | W | 114 | 66.956 | 9.523 | −15.205 | 1.00 | 44.06 | O |
| ATOM | 2386 | O | HOH | W | 115 | 71.948 | 39.028 | 3.510 | 1.00 | 48.92 | O |
| ATOM | 2387 | O | HOH | W | 116 | 73.384 | 36.583 | 9.395 | 1.00 | 49.46 | O |
| ATOM | 2388 | O | HOH | W | 117 | 69.213 | 13.943 | 11.885 | 1.00 | 43.79 | O |
| ATOM | 2389 | O | HOH | W | 118 | 92.376 | 29.991 | −13.421 | 1.00 | 50.66 | O |
| ATOM | 2390 | O | HOH | W | 119 | 71.748 | 33.616 | 8.364 | 1.00 | 44.60 | O |
| ATOM | 2391 | O | HOH | W | 120 | 72.751 | 30.625 | 9.138 | 1.00 | 54.54 | O |
| ATOM | 2392 | O | HOH | W | 121 | 44.373 | 14.896 | 1.763 | 1.00 | 54.90 | O |
| ATOM | 2393 | O | HOH | W | 122 | 72.331 | 37.663 | 5.252 | 1.00 | 54.91 | O |
| ATOM | 2394 | O | HOH | W | 123 | 85.766 | 37.929 | 7.966 | 1.00 | 45.42 | O |
| ATOM | 2395 | O | HOH | W | 124 | 82.375 | 46.624 | −12.952 | 1.00 | 49.98 | O |
| ATOM | 2396 | O | HOH | W | 125 | 69.185 | 5.514 | −10.117 | 1.00 | 57.15 | O |
| ATOM | 2397 | O | HOH | W | 126 | 72.843 | 16.943 | −2.415 | 1.00 | 48.35 | O |
| ATOM | 2398 | O | HOH | W | 127 | 58.459 | 18.549 | −11.193 | 1.00 | 68.47 | O |
| ATOM | 2399 | O | HOH | W | 128 | 64.272 | 33.293 | −12.839 | 1.00 | 48.86 | O |
| ATOM | 2400 | O | HOH | W | 129 | 59.782 | 37.121 | −16.253 | 1.00 | 59.29 | O |

TABLE 5
DISEASES ASSOCIATED WITH GENETIC DEFECTS IN KINASES

Serine/Threonine Protein Kinases
CYCLIN-DEPENDENT KINASE 7; CDK7
DYSTROPHIA MYOTONICA; DM
MINIBRAIN (DROSOPHILA) HOMOLOG; MNBH
RAC SERINE/THREONINE PROTEIN KINASE
SERINE-THREONINE PROTEIN KINASE N; PKN
SERINE/THREONINE PROTEIN KINASE 2; STK2
PROTEIN KINASE, DNA-ACTIVATED, CATALYTIC SUBUNIT; PRKDC
ZIPPER PROTEIN KINASE; ZPK
PROTEIN-TYROSINE KINASE STY
BRUTON AGAMMAGLOBULINEMIA TYROSINE KINASE; BTK
MKN28 KINASE
PROTEIN KINASE, X-LINKED; PRKX
ELK-RELATED TYROSINE KINASE; ERK
RIBOSOMAL PROTEIN S6 KINASE, 90 KD, POLYPEPTIDE 3; RPS6KA3
GLYCOGEN STORAGE DISEASE VIII
DEATH-ASSOCIATED PROTEIN KINASE 1; DAPK1
PCTAIRE PROTEIN KINASE 1; PCTK1
PROTEIN KINASE, INTERFERON-INDUCIBLE DOUBLE-STRANDED RNA; PRKR
ACTIVIN A RECEPTOR, TYPE II-LIKE KINASE 1; ACVRLK1
PROTEIN KINASE, cAMP-DEPENDENT, CATALYTIC, ALPHA; PRKACA
PROTEIN KINASE, Y-LINKED; PRKY
G PROTEIN-COUPLED RECEPTOR KINASE 2 (DROSOPHILA)-LIKE; GPRK2L
PROTEIN KINASE C, THETA FORM; PRKCQ
LIM DOMAIN KINASE 1; LIMK1
PHOSPHOGLYCERATE KINASE 1; PGK1
LIM DOMAIN KINASE 2; LIMK2
C-JUN KINASE
ACTIVIN A RECEPTOR, TYPE II-LIKE KINASE 2; ACVRLK2
JANUS KINASE 1; JAK1
ELKL MOTIF KINASE; EMK1
MALE GERM CELL-ASSOCIATED KINASE; MAK
CASEIN KINASE 2, ALPHA-PRIME SUBUNIT; CSNK2A2
CASEIN KINASE 2, BETA POLYPEPTIDE; CSNK2B
CASEIN KINASE 2, ALPHA 1 POLYPEPTIDE; CSNK2A1
LEUKEMIA, CHRONIC MYELOID; CML
RET PROTO-ONCOGENE; RET
HEMATOPOIETIC PROGENITOR KINASE 1
CONSERVED HELIX-LOOP-HELIX UBIQUITOUS KINASE; CHUK
CASEIN KINASE 1, DELTA; CSNK1D
CASEIN KINASE 1, EPSILON; CSNK1E
DEATH-ASSOCIATED PROTEIN; DAP
V-AKT MURINE THYMOMA VIRAL ONCOGENE HOMOLOG 1; AKT1
TUMOR PROTEIN p53; TP53
PROTEIN PHOSPHATASE 1, REGULATORY (INHIBITOR) SUBUNIT 2; PPP1R2
SEVERE COMBINED IMMUNODEFICIENCY DISEASE-1; SCID1
HYPERLIPOPROTEINEMIA, TYPE I
COLLAGEN, TYPE I, ALPHA-1 CHAIN; COL1A1
UBIQUITIN-BINDING PROTEIN P62
TAY-SACHS DISEASE; TSD
CYSTIC FIBROSIS; CF
p160-ROCK
BCL2-ASSOCIATED ATHANOGENE 1; BAG1
HYPOXANTHINE GUANINE PHOSPHORIBOSYLTRANSFERASE 1; HPRT1
ONCOGENE PIM-1; PIM1; PIM
CARTILAGE-DERIVED MORPHOGENETIC PROTEIN 1
GALACTOSEMIA
GRANULOMATOUS DISEASE, CHRONIC, AUTOSOMAL CYTOCHROME-b-POSITIVE FORM I
TRANSFORMING GROWTH FACTOR-BETA RECEPTOR, TYPE II; TGFBR2
RHODOPSIN; RHO
MOTHERS AGAINST DECAPENTAPLEGIC (DROSOPHILA) HOMOLOG 2; MADH2
TRIPLE FUNCTIONAL DOMAIN; TRIO
TRANSFORMING GROWTH FACTOR-BETA RECEPTOR, TYPE I; TGFBR1
GENERAL TRANSCRIPTION FACTOR IIH, POLYPEPTIDE 1; GTF2H1
ANTI-MULLERIAN HORMONE TYPE II RECEPTOR; AMHR2
V-RAF MURINE SARCOMA VIRAL ONCOGENE HOMOLOG B1; BRAF
BONE MORPHOGENETIC RECEPTOR TYPE II; BMPR2
GLYCOGEN STORAGE DISEASE V
COMPLEX IV, CYTOCHROME c OXIDASE SUBUNIT III
V-RAF MURINE SARCOMA 3611 VIRAL ONCOGENE HOMOLOG 1; ARAF1
GAUCHER DISEASE, TYPE I; GD I
V-RAF MURINE SARCOMA 3611 VIRAL ONCOGENE HOMOLOG 2; ARAF2
CYCLIN A; CCNA
ANEMIA, HYPOCHROMIC; ANH1
GRANULOMATOUS DISEASE, CHRONIC; CGD
ACTIVIN A RECEPTOR, TYPE I; ACVR1
Serine Protein Kinases
CYCLIN-DEPENDENT KINASE 7; CDK7
DYSTROPHIA MYOTONICA; DM
MINIBRAIN (DROSOPHILA) HOMOLOG; MNBH
RAC SERINE/THREONINE PROTEIN KINASE
SERINE-THREONINE PROTEIN KINASE N; PKN
PROTEIN KINASE, SERINE/ARGININE-SPECIFIC
PROTEIN SERINE KINASE H1; PSKH1
SERINE/THREONINE PROTEIN KINASE 2; STK2
PROTEIN KINASE, DNA-ACTIVATED, CATALYTIC SUBUNIT; PRKDC
ZIPPER PROTEIN KINASE; ZPK
PROTEIN-TYROSINE KINASE STY
SYK-RELATED TYROSINE KINASE; SRK
BRUTON AGAMMAGLOBULINEMIA TYROSINE KINASE; BTK
MKN28 KINASE
NON-METASTATIC CELLS 1, PROTEIN EXPRESSED IN; NME1
ELK-RELATED TYROSINE KINASE; ERK
PROTEIN KINASE, X-LINKED; PRKX
FIBROBLAST GROWTH FACTOR RECEPTOR-2; FGFR2
RIBOSOMAL PROTEIN S6 KINASE, 90 KD, POLYPEPTIDE 3; RPS6KA3
PCTAIRE PROTEIN KINASE 1; PCTK1
DEATH-ASSOCIATED PROTEIN KINASE 1; DAPK1
ACTIVIN A RECEPTOR, TYPE II-LIKE KINASE 1; ACVRLK1
GLYCOGEN STORAGE DISEASE VIII
PROTEIN KINASE, INTERFERON-INDUCIBLE DOUBLE-STRANDED RNA; PRKR
PROTEIN KINASE, Y-LINKED; PRKY
G PROTEIN-COUPLED RECEPTOR KINASE 2 (DROSOPHILA)-LIKE; GPRK2L
LIM DOMAIN KINASE 1; LIMK1
PROTEIN KINASE, cAMP-DEPENDENT, CATALYTIC, ALPHA; PRKACA
PROTEIN KINASE C, THETA FORM; PRKCQ
C-JUN KINASE
PHOSPHOGLYCERATE KINASE 1; PGK1
LIM DOMAIN KINASE 2; LIMK2
ACTIVIN A RECEPTOR, TYPE II-LIKE KINASE 2; ACVRLK2
JANUS KINASE 1; JAK1
INSULIN RECEPTOR; INSR
CASEIN KINASE 2, ALPHA 1 POLYPEPTIDE; CSNK2A1
CASEIN KINASE 2, BETA POLYPEPTIDE; CSNK2B
CASEIN KINASE 2, ALPHA-PRIME SUBUNIT; CSNK2A2
ATAXIA-TELANGIECTASIA; AT
MALE GERM CELL-ASSOCIATED KINASE; MAK
ELKL MOTIF KINASE; EMK1
LEUKEMIA, CHRONIC MYELOID; CML
HEMATOPOIETIC PROGENITOR KINASE 1
RET PROTO-ONCOGENE; RET
CASEIN KINASE 1, DELTA; CSNK1D
CASEIN KINASE 1, EPSILON; CSNK1E
CONSERVED HELIX-LOOP-HELIX UBIQUITOUS KINASE; CHUK
HYPERCHOLESTEROLEMIA, FAMILIAL; FHC
TUMOR PROTEIN p53; TP53
TAY-SACHS DISEASE; TSD
DEATH-ASSOCIATED PROTEIN; DAP
V-AKT MURINE THYMOMA VIRAL ONCOGENE HOMOLOG 1; AKT1
COLLAGEN, TYPE I, ALPHA-1 CHAIN; COL1A1
SEVERE COMBINED IMMUNODEFICIENCY DISEASE-1; SCID1
ADENOMATOUS POLYPOSIS OF THE COLON; APC
PROTEIN PHOSPHATASE 1, REGULATORY (INHIBITOR) SUBUNIT 2; PPP1R2
UBIQUITIN-BINDING PROTEIN P62
MUSCULAR DYSTROPHY, TARDIVE, DREIFUSS-EMERY TYPE, WITH CONTRACTURES
CAVEOLIN, CAVEOLAE PROTEIN, 22-KD; CAV
FIBROBLAST GROWTH FACTOR RECEPTOR-3; FGFR3
CYSTIC FIBROSIS; CF
HYPOXANTHINE GUANINE PHOSPHORIBOSYLTRANSFERASE 1; HPRT1
GROWTH FACTOR RECEPTOR-BOUND PROTEIN-10; GRB10
HYPERLIPOPROTEINEMIA, TYPE I
CREB-BINDING PROTEIN; CREBBP
THYROID AUTOANTIGEN 70 KD; G22P1
ONCOGENE PIM-1; PIM1; PIM
SIGNAL TRANSDUCER AND ACTIVATOR OF TRANSCRIPTION 3; STAT3
CYCLIC-AMP-RESPONSE-ELEMENT-BINDING PROTEIN-1; CREB1
BCL2-ASSOCIATED ATHANOGENE 1; BAG1
P32 SPLICING FACTOR SF2 ASSOCIATED PROTEIN
CARTILAGE-DERIVED MORPHOGENETIC PROTEIN 1
p160-ROCK
GROWTH FACTOR RECEPTOR-BOUND PROTEIN-14; GRB14
THROMBASTHENIA OF GLANZMANN AND NAEGELI
INSULIN RECEPTOR SUBSTRATE 1; IRS1
GRANULOMATOUS DISEASE, CHRONIC, AUTOSOMAL CYTOCHROME-b-POSITIVE FORM I
GUANYLATE CYCLASE 2D, MEMBRANE; GUC2D
GALACTOSEMIA
RHODOPSIN; RHO
TRANSFORMING GROWTH FACTOR-BETA RECEPTOR, TYPE II; TGFBR2
ICHTHYOSIS, X-LINKED
TRIPLE FUNCTIONAL DOMAIN; TRIO
MOTHERS AGAINST DECAPENTAPLEGIC (DROSOPHILA) HOMOLOG 2; MADH2
MCF.2 CELL LINE DERIVED TRANSFORMING SEQUENCE; MCF2
ESTROGEN RECEPTOR; ESR
V-RAF MURINE SARCOMA 3611 VIRAL ONCOGENE HOMOLOG 1; ARAF1
V-RAF MURINE SARCOMA 3611 VIRAL ONCOGENE HOMOLOG 2; ARAF2
BONE MORPHOGENETIC RECEPTOR TYPE II; BMPR2
GAUCHER DISEASE, TYPE I; GD I
TRANSFORMING GROWTH FACTOR-BETA RECEPTOR, TYPE I; TGFBR1
GENERAL TRANSCRIPTION FACTOR IIH, POLYPEPTIDE 1; GTF2H1
CHYMOTRYPSIN-LIKE PROTEASE; CTRL
GLYCOGEN STORAGE DISEASE V
CYCLIN A; CCNA
LETHAL GIANT LARVAE (DROSOPHILA) HOMOLOG 1; LLGL1
GRANULOMATOUS DISEASE, CHRONIC; CGD
CASEIN, BETA; CSN2
DENTIN MATRIX ACIDIC PHOSPHOPROTEIN 1; DMP1
TREACHER COLLINS-FRANCESCHETTI SYNDROME 1; TCOF1
COMPLEX IV, CYTOCHROME c OXIDASE SUBUNIT III
DIABETES MELLITUS, AUTOSOMAL DOMINANT
V-RAF MURINE SARCOMA VIRAL ONCOGENE HOMOLOG B1; BRAF
ANEMIA, HYPOCHROMIC; ANH1
ANTI-MULLERIAN HORMONE TYPE II RECEPTOR; AMHR2
PLASMINOGEN ACTIVATOR INHIBITOR, TYPE 2; PAI2
ACTIVIN A RECEPTOR, TYPE I; ACVR1
Threonine Protein Kinases
CYCLIN-DEPENDENT KINASE 7; CDK7
DYSTROPHIA MYOTONICA; DM
MINIBRAIN (DROSOPHILA) HOMOLOG; MNBH
RAC SERINE/THREONINE PROTEIN KINASE
SERINE-THREONINE PROTEIN KINASE N; PKN
SERINE/THREONINE PROTEIN KINASE 2; STK2
PROTEIN KINASE, DNA-ACTIVATED, CATALYTIC SUBUNIT; PRKDC
ZIPPER PROTEIN KINASE; ZPK
PROTEIN-TYROSINE KINASE STY
PROTEIN KINASE, MITOGEN-ACTIVATED, KINASE 1; PRKMK1
BRUTON AGAMMAGLOBULINEMIA TYROSINE KINASE; BTK
MKN28 KINASE
PROTEIN KINASE, X-LINKED; PRKX
ELK-RELATED TYROSINE KINASE; ERK
GLYCOGEN STORAGE DISEASE VIII
PYRUVATE KINASE DEFICIENCY OF ERYTHROCYTE
RIBOSOMAL PROTEIN S6 KINASE, 90 KD, POLYPEPTIDE 3; RPS6KA3
DEATH-ASSOCIATED PROTEIN KINASE 1; DAPK1
PCTAIRE PROTEIN KINASE 1; PCTK1
PROTEIN KINASE, INTERFERON-INDUCIBLE DOUBLE-STRANDED RNA; PRKR
ACTIVIN A RECEPTOR, TYPE II-LIKE KINASE 1; ACVRLK1
PROTEIN KINASE, Y-LINKED; PRKY
G PROTEIN-COUPLED RECEPTOR KINASE 2 (DROSOPHILA)-LIKE; GPRK2L
PROTEIN KINASE, cAMP-DEPENDENT, CATALYTIC, ALPHA; PRKACA
PROTEIN KINASE C, THETA FORM; PRKCQ
MUSCULAR DYSTROPHY, PSEUDOHYPERTROPHIC PROGRESSIVE, DUCHENNE AND BECKER
TYPES; DMD
LIM DOMAIN KINASE 1; LIMK1
PHOSPHOGLYCERATE KINASE 1; PGK1
LIM DOMAIN KINASE 2; LIMK2
C-JUN KINASE
ACTIVIN A RECEPTOR, TYPE II-LIKE KINASE 2; ACVRLK2
JANUS KINASE 1; JAK1
CASEIN KINASE 2, ALPHA 1 POLYPEPTIDE; CSNK2A1
CASEIN KINASE 2, BETA POLYPEPTIDE; CSNK2B
MALE GERM CELL-ASSOCIATED KINASE; MAK
MEVALONICACIDURIA
ELKL MOTIF KINASE; EMK1
CASEIN KINASE 2, ALPHA-PRIME SUBUNIT; CSNK2A2
MAP KINASE KINASE 6
CASEIN KINASE 1, EPSILON; CSNK1E
CASEIN KINASE 1, DELTA; CSNK1D
LEUKEMIA, CHRONIC MYELOID; CML
CONSERVED HELIX-LOOP-HELIX UBIQUITOUS KINASE; CHUK
HEMATOPOIETIC PROGENITOR KINASE 1
RET PROTO-ONCOGENE; RET
PROTEIN PHOSPHATASE 1, REGULATORY (INHIBITOR) SUBUNIT 2; PPP1R2
DUAL SPECIFICITY PHOSPHATASE 1; DUSP1
V-AKT MURINE THYMOMA VIRAL ONCOGENE HOMOLOG 1; AKT1
DEATH-ASSOCIATED PROTEIN; DAP
TUMOR PROTEIN p53; TP53
GLYCOGEN STORAGE DISEASE II
SEVERE COMBINED IMMUNODEFICIENCY DISEASE-1; SCID1
UBIQUITIN-BINDING PROTEIN P62
HYPERLIPOPROTEINEMIA, TYPE I
BCL2-ASSOCIATED ATHANOGENE 1; BAG1
ONCOGENE PIM-1; PIM1; PIM
HYPOXANTHINE GUANINE PHOSPHORIBOSYLTRANSFERASE 1; HPRT1
p160-ROCK
CARTILAGE-DERIVED MORPHOGENETIC PROTEIN 1
CYSTIC FIBROSIS; CF
TAY-SACHS DISEASE; TSD
COLLAGEN, TYPE I, ALPHA-1 CHAIN; COL1A1
GRANULOMATOUS DISEASE, CHRONIC, AUTOSOMAL CYTOCHROME-b-POSITIVE FORM I
NEUROMATA, MUCOSAL, WITH ENDOCRINE TUMORS
TRANSFORMING GROWTH FACTOR-BETA RECEPTOR, TYPE II; TGFBR2
GALACTOSEMIA
MOTHERS AGAINST DECAPENTAPLEGIC (DROSOPHILA) HOMOLOG 2; MADH2
TRIPLE FUNCTIONAL DOMAIN; TRIO
ANTI-MULLERIAN HORMONE TYPE II RECEPTOR; AMHR2
BONE MORPHOGENETIC RECEPTOR TYPE II; BMPR2
V-RAF MURINE SARCOMA VIRAL ONCOGENE HOMOLOG B1; BRAF
CYCLIN A; CCNA
COMPLEX IV, CYTOCHROME c OXIDASE SUBUNIT III
RHODOPSIN; RHO
V-RAF MURINE SARCOMA 3611 VIRAL ONCOGENE HOMOLOG 1; ARAF1
GRANULOMATOUS DISEASE, CHRONIC; CGD
TRANSFORMING GROWTH FACTOR-BETA RECEPTOR, TYPE I; TGFBR1
ANEMIA, HYPOCHROMIC; ANH1
GENERAL TRANSCRIPTION FACTOR IIH, POLYPEPTIDE 1; GTF2H1
GLYCOGEN STORAGE DISEASE V
GAUCHER DISEASE, TYPE I; GD I
ADENINE PHOSPHORIBOSYLTRANSFERASE; APRT
V-RAF MURINE SARCOMA 3611 VIRAL ONCOGENE HOMOLOG 2; ARAF2
ACTIVIN A RECEPTOR, TYPE I; ACVR1
Tyrosine Protein Kinases
NEUROTROPHIC TYROSINE KINASE, RECEPTOR, TYPE 1; NTRK1
PROTEIN-TYROSINE KINASE, CYTOPLASMIC; PTK2
SYK-RELATED TYROSINE KINASE; SRK
PROTEIN TYROSINE KINASE CTK; CTK
TYRO3 PROTEIN TYROSINE KINASE; TYRO3
BRUTON AGAMMAGLOBULINEMIA TYROSINE KINASE; BTK
LEUKOCYTE TYROSINE KINASE; LTK
PROTEIN-TYROSINE KINASE SYK; SYK
PROTEIN-TYROSINE KINASE STY
TEK TYROSINE KINASE, ENDOTHELIAL; TEK
ELK-RELATED TYROSINE KINASE; ERK
TYROSINE KINASE WITH IMMUNOGLOBULIN AND EGF FACTOR HOMOLOGY DOMAINS; TIE
PROTEIN TYROSINE KINASE TKF
NEUROTROPHIC TYROSINE KINASE, RECEPTOR, TYPE 3; NTRK3
MIXED-LINEAGE PROTEIN KINASE-3; MLK3
PROTEIN KINASE, MITOGEN-ACTIVATED 4; PRKM4
PROTEIN KINASE, MITOGEN-ACTIVATED 1; PRKM1
PROTEIN TYROSINE KINASE PTK7; PTK7
PROTEIN TYROSINE KINASE EEK
MINIBRAIN (DROSOPHILA) HOMOLOG; MNBH
BONE MARROW KINASE, X-LINKED; BMX
EPH-LIKE TYROSINE KINASE 1; ETK1
MACROPHAGE STIMULATING 1 RECEPTOR; MST1R
BTK-ASSOCIATED PROTEIN, 135 KD
LYMPHOCYTE-SPECIFIC PROTEIN TYROSINE KINASE; LCK
FIBROBLAST GROWTH FACTOR RECEPTOR-2; FGFR2
PROTEIN TYROSINE KINASE-3; TYK3; FER
PROTEIN TYROSINE KINASE TXK; TXK
TEC PROTEIN TYROSINE KINASE; TEC
PROTEIN TYROSINE KINASE-2; TYK2
EPH-RELATED RECEPTOR TYROSINE KINASE LIGAND 1; EPLG1
T-CELL TYROSINE KINASE EMT; EMT
EPH TYROSINE KINASE 1; EPHT1
ZONA PELLUCIDA RECEPTOR TYROSINE KINASE, 95 KD; ZRK
PROTEIN KINASE, MITOGEN-ACTIVATED, KINASE 1; PRKMK1
EPH TYROSINE KINASE 3; EPHT3
GROWTH ARREST-SPECIFIC GENE-6; GAS6
KINASE INSERT DOMAIN RECEPTOR; KDR
AXL RECEPTOR TYROSINE KINASE; AXL
FIBROBLAST GROWTH FACTOR RECEPTOR-1; FGFR1
V-ERB-B2 AVIAN ERYTHROBLASTIC LEUKEMIA VIRAL ONCOGENE HOMOLOG 2; ERBB2
FMS-LIKE TYROSINE KINASE-3; FLT3
NEUROEPITHELIAL TYROSINE KINASE; NEP
NEUROTROPHIC TYROSINE KINASE RECEPTOR-RELATED 3; NTRKR3
EPH-RELATED RECEPTOR TYROSINE KINASE LIGAND 5; EPLG5
NEUROTROPHIC TYROSINE KINASE, RECEPTOR, TYPE 2; NTRK2
RYK RECEPTOR-LIKE TYROSINE KINASE
TYROSINE KINASE, B-LYMPHOCYTE SPECIFIC; BLK
EPH TYROSINE KINASE 2; EPHT2
EPH-RELATED RECEPTOR TYROSINE KINASE LIGAND 2; EPLG2
GLYCOGEN STORAGE DISEASE VIII
EPH-RELATED RECEPTOR TYROSINE KINASE LIGAND 7; EPLG7
JANUS KINASE 1; JAK1
FMS-RELATED TYROSINE KINASE-1; FLT1
PROTEIN KINASE, cAMP-DEPENDENT, REGULATORY, TYPE I, ALPHA; PRKAR1A
WEE-1 TYROSINE KINASE; WEE1
EPH-LIKE TYROSINE KINASE 2; ETK2
RECEPTOR TYROSINE KINASE MuSK
INSULIN RECEPTOR; INSR
JANUS KINASE 3 JAK3
FMS-RELATED TYROSINE KINASE-3 LIGAND
PROTEIN KINASE C, BETA 1; PRKCB1
TYROSINE KINASE-TYPE CELL SURFACE RECEPTOR HER3; HER3
JANUS KINASE 2; JAK2
LIM DOMAIN KINASE 1; LIMK1
DUAL SPECIFICITY PHOSPHATASE 1; DUSP1
MUSCULAR DYSTROPHY, PSEUDOHYPERTROPHIC PROGRESSIVE, DUCHENNE AND BECKER
TYPES; DMD
HEMOPOIETIC CELL KINASE; HCK
TYROSINE 3-MONOOXYGENASE/TRYPTOPHAN 5-MONOOXYGENASE ACTIVATION PROTEIN,
ETA POLYPEPTIDE; YWHAH
RET PROTO-ONCOGENE; RET
TYROSINE 3-MONOOXYGENASE/TRYPTOPHAN 5-MONOOXYGENASE ACTIVATION PROTEIN,
ZETA POLYPEPTIDE; YWHAZ
TYROSINE 3-MONOOXYGENASE/TRYPTOPHAN 5-MONOOXYGENASE ACTIVATION PROTEIN,
BETA POLYPEPTIDE; YWHAB
HEPATOMA TRANSMEMBRANE KINASE; HTK
MAP KINASE KINASE 6
PHOSPHATIDYLINOSITOL 3-KINASE, CATALYTIC, ALPHA POLYPEPTIDE; PIK3CA
CYCLIN-DEPENDENT KINASE INHIBITOR 3; CDKN3
DIACYLGLYCEROL KINASE, DELTA, 130 KD
PROTEIN-TYROSINE PHOSPHATASE, NONRECEPTOR TYPE, 13; PTPN13
ABELSON MURINE LEUKEMIA VIRAL ONCOGENE HOMOLOG 1; ABL1
DIACYLGLYCEROL KINASE, ALPHA; DAGK1
FOCAL ADHESION KINASE 2
EPITHELIAL DISCOIDIN DOMAIN RECEPTOR 1; EDDR1
ANAPLASTIC LYMPHOMA KINASE; ALK
PHOSPHATIDYLINOSITOL 3-KINASE, CATALYTIC, GAMMA POLYPEPTIDE; PIK3CG
PHOSPHATIDYLINOSITOL 3-KINASE REGULATORY SUBUNIT; PIK3R1
EPH HOMOLOGY KINASE-1; EHK1
V-KIT HARDY-ZUCKERMAN 4 FELINE SARCOMA VIRAL ONCOGENE HOMOLOG; KIT
FIBROBLAST GROWTH FACTOR RECEPTOR-3; FGFR3
VASCULAR ENDOTHELIAL GROWTH FACTOR C; VEGFC
MACROPHAGE STIMULATING 1; MST1
HYPERCHOLESTEROLEMIA, FAMILIAL; FHC
EPIDERMAL GROWTH FACTOR RECEPTOR; EGFR
S100 CALCIUM-BINDING PROTEIN A10; S100A10
NEUROFIBROMATOSIS, TYPE I; NF1
ONCOGENE TRK
LEUKEMIA, CHRONIC MYELOID; CML
GROWTH FACTOR RECEPTOR-BOUND PROTEIN-7; GRB7
S100 CALCIUM-BINDING PROTEIN A4; S100A4
RAS p21 PROTEIN ACTIVATOR; RASA2
ADENOMATOUS POLYPOSIS OF THE COLON; APC
MET PROTO-ONCOGENE; MET
SIGNAL TRANSDUCER AND ACTIVATOR OF TRANSCRIPTION 3; STAT3
smg GDS-ASSOCIATED PROTEIN
UBIQUITIN-BINDING PROTEIN P62
LYMPHOCYTE CYTOSOLIC PROTEIN 2; LCP2
EPIDERMAL GROWTH FACTOR RECEPTOR PATHWAY SUBSTRATE-15; EPS15
GROWTH FACTOR RECEPTOR-BOUND PROTEIN-10; GRB10
GLIAL CELL LINE-DERIVED NEUROTROPHIC FACTOR RECEPTOR-ALPHA; GDNFRA
SHC TRANSFORMING PROTEIN; SHC1
CYSTIC FIBROSIS; CF
TROPOMYOSIN 3; TPM3
CELL DIVISION CYCLE 2, G1 TO S AND G2 TO M; CDC2
MUSCULAR DYSTROPHY, LIMB GIRDLE, TYPE 2C; LGMD2C
ASH PROTEIN
TAY-SACHS DISEASE; TSD
AGRIN; AGRN
S100 CALCIUM-BINDING PROTEIN A6; S100A6
AGRANULOCYTOSIS, INFANTILE GENETIC
TRIPLE FUNCTIONAL DOMAIN; TRIO
HYPOXANTHINE GUANINE PHOSPHORIBOSYLTRANSFERASE 1; HPRT1
CYTOVILLIN
GOLGI APPARATUS PROTEIN 1; GLG1
GROWTH FACTOR RECEPTOR-BOUND PROTEIN-14; GRB14
V-FES FELINE SARCOMA VIRAL/V-FPS FUJINAMI AVIAN SARCOMA VIRAL ONCOGENE
HOMOLOG; FES
TRANSLOCATED PROMOTER REGION
P32 SPLICING FACTOR SF2 ASSOCIATED PROTEIN
CARTILAGE-DERIVED MORPHOGENETIC PROTEIN 1
PAIRED BOX HOMEOTIC GENE 5; PAX5
INSULIN RECEPTOR SUBSTRATE 1; IRS1
SON OF SEVENLESS (DROSOPHILA) HOMOLOG 2; SOS2
PHOSPHATIDYLINOSITOL GLYCAN CLASS A; PIGA
ICHTHYOSIS, X-LINKED
RHODOPSIN; RHO
SEVERE COMBINED IMMUNODEFICIENCY DISEASE, AUTOSOMAL RECESSIVE, T-
NEGATIVE/B-POSITIVE TYPE
CAS-BR-M (MURINE) ECOTROPIC RETROVIRAL TRANSFORMING SEQUENCE; CBL
TRANSFORMING GROWTH FACTOR-BETA RECEPTOR, TYPE II; TGFBR2
COLONY-STIMULATING FACTOR-1 RECEPTOR; CSF1R
PHOSPHODIESTERASE I/NUCLEOTIDE PYROPHOSPHATASE 1; PDNP1
NUCLEOPHOSMIN; NPM1
ADDUCIN 1; ADD1
NEUROMATA, MUCOSAL, WITH ENDOCRINE TUMORS
HYALURONAN-MEDIATED MOTILITY RECEPTOR; HMMR
ESTROGEN RECEPTOR; ESR
SRC-LIKE ADAPTER; SLA
BECKWITH-WIEDEMANN SYNDROME; BWS
TYLOSIS WITH ESOPHAGEAL CANCER; TOC
PLACENTAL GROWTH FACTOR; PGF
ETS VARIANT GENE 6; ETV6
MEMBRANE COMPONENT, CHROMOSOME 6, POLYPEPTIDE 2; M6P2
INTERLEUKIN-4; IL4
GARDNER-RASHEED FELINE SARCOMA VIRAL (V-FGR) ONCOGENE; FGR
FIBROBLAST GROWTH FACTOR-8; FGF8
SORTING NEXIN-1; SNX1
TRANSCRIPTION FACTOR 1, HEPATIC; TCF1
HEPATOCYTE GROWTH FACTOR; HGF
INTERLEUKIN-6 RECEPTOR; IL6R
V-YES-1 YAMAGUCHI SARCOMA VIRAL ONCOGENE; YES1
ENDOGLIN; ENG
THANATOPHORIC DYSPLASIA WITH KLEEBLATTSCHAEDEL
HEMATOPOIETIC CELL-SPECIFIC LYN SUBSTRATE 1; HCLS1
GENERAL TRANSCRIPTION FACTOR IIH, POLYPEPTIDE 1; GTF2H1
V-SIS PLATELET-DERIVED GROWTH FACTOR BETA POLYPEPTIDE; PDGFB
DIABETES MELLITUS, AUTOSOMAL DOMINANT
PROGRAMMED CELL DEATH 1; PDCD1
TRANSFORMING GROWTH FACTOR-BETA RECEPTOR, TYPE I; TGFBR1
EPIDERMAL GROWTH FACTOR RECEPTOR PATHWAY SUBSTRATE-8; EPS8
VASCULAR ENDOTHELIAL GROWTH FACTOR; VEGF
CELL ADHESION REGULATOR; CAR
ANEMIA, CONGENITAL HYPOPLASTIC, OF BLACKFAN AND DIAMOND
GAUCHER DISEASE, TYPE I; GD I
MAPLE SYRUP URINE DISEASE; MSUD
MCF.2 CELL LINE DERIVED TRANSFORMING SEQUENCE; MCF2
GRANULOMATOUS DISEASE, CHRONIC; CGD
ANGIOPOIETIN 2; ANGPT2
HYPOGAMMAGLOBULINEMIA AND ISOLATED GROWTH HORMONE DEFICIENCY, X-LINKED
GLIAL CELL LINE-DERIVED NEUROTROPHIC FACTOR RECEPTOR-BETA
H4 GENE

1. A kinase scaffold library comprising at least one set of compounds, each set consisting essentially of a plurality of compounds of a chemical structure selected from the group consisting of Formula I, II, III, IV, V, VI, and VII.

2. The library of claim 1, wherein said set comprises at least 50 different compounds.

3. The library of claim 1, wherein said library comprises at least 3 said sets.

4. The library of claim 1, wherein a majority of compounds in said set have been demonstrated to bind to one or more kinases.

5. The library of claim 4, wherein said kinases comprise a plurality of kinases selected from the group consisting of PIM-1, Pyk2, c-Abl, Her2, cMet, VEGFR, EGFR, cKit, Pkcβ, p38, Cdk2, Akt, Gsk3β.

6. The library of claim 1, wherein each said compound is in a separate well in a plate or plurality of plates.

7. A system for fitting compounds in binding sites of one or more protein kinases, comprising
an electronic kinase scaffold library comprising at least one collection of electronic representations of compounds of a chemical structure selected from the group consisting of Formula I, II, III, IV, V, VI, and VII, wherein said kinase scaffold library is embedded in a computer memory device, wherein said electronic representations of said compounds can be selectively retrieved and functionally connected with computer software adapted to fit electronic representations of compounds in an electronic representation of a binding site of a kinase.

8. The system of claim 7, further comprising at least one electronic representation of a kinase binding site embedded in computer memory such that said electronic representation of a kinase binding site can be functionally connected with said computer software.

9. The system of claim 8, where said electronic representation of kinase binding sites comprises electronic representations of binding sites of a plurality of kinases selected from the group consisting of PIM-1, Pyk2, c-Abl, Her2, cMet, VEGFR, EGFR, cKit, Pkcβ, p38, Cdk2, Akt, Gsk3β.

10. A method for obtaining improved ligands binding to a protein kinase, comprising
determining whether a derivative of a compound of Formula I, II, III, IV, V, VI, or VII binds to said kinase with greater affinity or greater specificity or both than said compound, wherein binding with greater affinity or greater specificity or both indicates that said derivative is an improved ligand.

11. The method of claim 10, wherein said derivative has at least 10-fold greater affinity or specificity or both than said compound.

12. The method of claim 10, wherein said derivative has at least 100-fold greater affinity or specificity or both.

13. The method of claim 10, wherein said kinase is selected from the group consisting of PIM-1, Pyk2, c-Abl, Her2, cMet, VEGFR, EGFR, cKit, Pkcβ, p38, Cdk2, Akt, Gsk3β.

14. A method for developing ligands specific for a particular kinase, comprising
determining whether a derivative of a compound of Formula I, II, III, IV, V, VI, or VII that binds to a plurality of kinases has greater specificity for said particular kinase than said compound.

15. The method of claim 14, wherein said compound binds to said kinase with an affinity at least 10-fold greater than for binding to any of said plurality of kinases.

16. The method of claim 15, wherein said compound interacts with at least one of PIM-1 residues 49, 52, 65, 67, 121, 128, and 186.

17. The method of claim 14, wherein said compound binds weakly to said plurality of kinases.

18. The method of claim 14, wherein said kinase is selected from the group consisting of PIM-1, Pyk2, c-Abl, Her2, cMet, VEGFR, EGFR, cKit, Pkcβ, p38, Cdk2, Akt, Gsk3β.

19. A method for developing ligands binding to a kinase, comprising
determining the orientation of at least one molecular scaffold of Formula I, II, III, IV, V, VI, or VII in co-crystals with said kinase; and
identifying chemical structures of said molecular scaffolds, that, when modified, alter the binding affinity or binding specificity or both between the molecular scaffold and said kinase; and
synthesizing a ligand wherein one or more of the chemical structures of the molecular scaffold is modified to provide a ligand that binds to said kinase with altered binding affinity or binding specificity or both.

20. The method of claim 19, wherein said molecular scaffold is a weak binding compound.

21. The method of claim 19, wherein said molecular scaffold binds to a plurality of kinases.

22. The method of claim 19, wherein said molecular scaffold interacts with at least 3 kinases selected from the group consisting of PIM-1, Pyk2, c-Abl, Her2, cMet, VEGFR, EGFR, cKit, Pkcβ, p38, Cdk2, Akt, and Gsk3β.

23. The method of claim 19, wherein said kinase is selected from the group consisting of PIM-1, Pyk2, c-Abl, Her2, cMet, cKit, Pkcβ, Cdk2, and Akt.

24. A method for developing ligands with increased specificity on a kinase, comprising
testing a derivative of a kinase binding compound of Formula I, II, III, IV, V, VI, or VII for increased specificity on said kinase, wherein increased specificity is indicative that said derivative is a ligand with increased specificity.

25. The method of claim 24, wherein said kinase binding compound binds to at least 5 different human kinases.

26. The method of claim 24, wherein said kinase binding compound binds to at least 10 different human kinases.

27. The method of claim 24, wherein said kinase is selected from the group consisting of PIM-1, Pyk2, c-Abl, Her2, cMet, VEGFR, EGFR, cKit, Pkcβ, p38, Cdk2, Akt, Gsk3β.

28. A method for identifying a ligand binding to a kinase, comprising
determining whether a derivative compound that includes a core structure selected from the group consisting of Formula I, II, III, IV, V, VI, and VII binds to said kinase with altered binding affinity or specificity or both as compared to the parent compound.

29. A co-crystal of a kinase and a binding compound of Formula I, II, III, IV, V, VI, or VII.

30. The co-crystal of claim 29, wherein said binding compound interacts with at least one of PIM-1 residues 49, 52, 65, 67, 121, 128, and 186.

31. The co-crystal of claim 29, wherein said kinase is selected from the group consisting of PIM-1, Pyk2, c-Abl, Her2, cMet, VEGFR, EGFR, cKit, Pkcβ, p38, Cdk2, Akt, Gsk3β.

32. The co-crystal of claim 29, wherein said co-crystal is in an X-ray beam.

33. A method for obtaining co-crystals of PIM-1 with a binding compound of Formula I, II, III, IV, V, VI, or VII, comprising subjecting PIM-1 protein at 5-20 mg/ml to crystallization conditions substantially equivalent to Hampton Screen 1 conditions 2, 7, 14, 17, 23, 25, 29, 36, 44, or 49 in the presence of binding compound for a time sufficient for crystal development.

34. The method of claim 33, wherein said binding compound is added to said protein to a final concentration of 0.5 to 1.0 mM.

35. The method of claim 34, wherein said binding compound is in a dimethyl sulfoxide solution.

36. The method of claim 33, wherein said crystallization condition is 0.4-0.9 M sodium acetate trihydrate pH 6.5, 0.1 M imidazole; or 0.2-0.7 M. sodium potassium tartrate, 00.1 M MES buffer pH 6.5.

37. A method for modulating kinase activity, comprising
contacting said kinase with a compound of Formula I, II, III, IV, V, VI, or VII.

38. The method of claim 37, wherein said kinase is selected from the group consisting of PIM-1, Pyk2, c-Abl, Her2, cMet, VEGFR, EGFR, cKit, Pkcβ, p38, Cdk2, Akt, Gsk3β.

39. The method of claim 37, wherein said compound is at a concentration of 200 μM or less.

40. A method for treating a patient suffering from a disease or condition characterized by abnormal kinase activity, comprising
administering to said patient a compound of Formula I, II, III, IV, V, VI, or VII active on said kinase.

41. The method of claim 40, wherein said kinase is selected from the group consisting of PIM-1, Pyk2, c-Abl, Her2, cMet, VEGFR, EGFR, cKit, Pkcβ, p38, Cdk2, Akt, Gsk3β.

42. The method of claim 40 wherein said disease or condition is a cancer.

43. The method of claim 40, wherein said disease or condition is an inflammatory disease or condition.

44. An electronic representation of a binding site of a kinase with a compound of a chemical structure selected from the group consisting of Formula I, II, III, IV, V, VI, and VII bound therein.

45. The electronic representation of claim 44, comprising a binding site surface contour.

46. The electronic representation of claim 44, comprising representations of the binding character of a plurality of conserved amino acid residues.

47. A method for identifying potential kinase binding compounds, comprising
fitting at least one electronic representations of a compound of Formula I, II, III, IV, V, VI, or VII in an electronic representation of a kinase binding site.

48. The method of claim 47, wherein said kinase is selected from the group consisting of PIM-1, Pyk2, c-Abl, Her2, cMet, VEGFR, EGFR, cKit, Pkcβ, p38, Cdk2, Akt, Gsk3β.

49. The method of claim 48, comprising
removing a computer representation of a compound complexed with said kinase and fitting a computer representation of a compound from a computer database with a computer representation of the active site of said kinase; and
identifying compounds that best fit said active site based on favorable geometric fit and energetically favorable complementary interactions as potential binding compounds.

50. The method of claim 48, comprising
modifying a computer representation of a compound complexed with said kinase by the deletion or addition or both of one or more chemical groups;
fitting a computer representation of a compound from a computer database with a computer representation of the active site of said kinase; and
identifying compounds that best fit said active site based on favorable geometric fit and energetically favorable complementary interactions as potential binding compounds.

51. The method of claim 48, comprising
removing a computer representation of a compound complexed with said kinase; and
searching a database for compounds having structural similarity to said compound using a compound searching computer program or replacing portions of said compound with similar chemical structures using a compound construction computer program.

52. A method for attaching a kinase binding compound to an attachment component, comprising
identifying energetically allowed sites for attachment of a said attachment component on a kinase binding compound of Formula I, II, III, IV, V, VI, or VII; and
attaching said compound or derivative thereof to said attachment component at said energetically allowed site.

53. The method of claim 52, wherein said attachment component is a linker for attachment to a solid phase medium, and said method further comprises attaching said compound or derivative to a solid phase medium through a linker attached at a said energetically allowed site.

54. The method of claim 52, wherein said kinase is selected from the group consisting of PIM-1, Pyk2, c-Abl, Her2, cMet, VEGFR, EGFR, cKit, Pkcβ, p38, Cdk2, Akt, Gsk3β.

55. The method of claim 52, wherein said kinase comprises conserved residues matching at least one of PIM-1 residues 49, 52, 65, 67, 121, 128, and 186.

56. The method of claim 53, wherein said linker is a traceless linker.

57. The method of claim 53, wherein said kinase binding compound or derivative thereof is synthesized on a said linker attached to said solid phase medium.

58. The method of claim 57, wherein a plurality of said compounds or derivatives are synthesized in combinatorial synthesis.

59. The method of claim 53, wherein attachment of said compound to said solid phase medium provides an affinity medium.

60. The method of claim 52, wherein said attachment component comprises a label.

61. The method of claim 60, wherein said label comprises a fluorophore.

62. A modified compound, comprising
a compound of Formula I, II, III, IV, V, VI, or VII, with a linker moiety attached thereto.

63. The compound of claim 62, wherein said linker is attached to an energetically allowed site for binding of said modified compound to a kinase selected from the group consisting of PIM-1, Pyk2, c-Abl, Her2, cMet, VEGFR, EGFR, cKit, Pkcβ, p38, Cdk2, Akt, Gsk3β.

64. The compound of claim 62, wherein said linker is attached to a solid phase.

65. The compound of claim 62, wherein said linker comprises or is attached to a label.

66. The compound of claim 62, wherein said linker is a traceless linker.

67. A method for developing a ligand for a kinase comprising conserved residues matching one or more of PIM-1 residues 49, 52, 65, 67, 121, 128, and 186, comprising
determining whether a compound of Formula I, II, III, IV, V, VI, or VII binds to said kinase.

68. The method of claim 67, wherein said kinase is selected from the group consisting of PIM-1, Pyk2, c-Abl, Her2, cMet, VEGFR, EGFR, cKit, Pkcβ, p38, Cdk2, Akt, Gsk3β.

69. The method of claim 67, wherein said kinase comprises conserved residues matching at least 2 of PIM-1 residues 49, 52, 65, 67, 121, 128, and 186.

70. The method of claim 67, wherein said kinase comprises conserved residues matching PIM-1 residues 49, 52, 65, 67, 121, 128, and 186.

71. The method of claim 67, further comprising determining whether said compound modulates said kinase.

72. The method of claim 67, wherein said determining comprises computer fitting said compound in a binding site of said kinase.

73. The method of claim 67, further comprising forming a co-crystal of said kinase and said compound.

74. The method of claim 73, further comprising determining the binding orientation of said compound with said kinase.
